SEQUENCE LISTING (492 sequences)

SEQ ID NO: 1 - protein, 10 aa, synthetic construct
  REGION 1..10: note = Description of Artificial Sequence: Syntheticpeptide
GFDIQDTYMH

SEQ ID NO: 2 - protein, 17 aa, synthetic construct
  REGION 1..17: note = Description of Artificial Sequence: Syntheticpeptide
RIDPASGHTKYDPKFQV

SEQ ID NO: 3 - protein, 17 aa, synthetic construct
  REGION 1..17: note = Description of Artificial Sequence: Syntheticpeptide
RIEPASGHIKYDPKFQG

SEQ ID NO: 4 - protein, 17 aa, synthetic construct
  REGION 1..17: note = Description of Artificial Sequence: Syntheticpeptide
RIDPASGHIKYDPKFQG

SEQ ID NO: 5 - protein, 17 aa, synthetic construct
  REGION 1..17: note = Description of Artificial Sequence: Syntheticpeptide
RIEPASGHIKYDPKFQV

SEQ ID NO: 6 - protein, 7 aa, synthetic construct
  REGION 1..7: note = Description of Artificial Sequence: Syntheticpeptide
SGGLPDV

SEQ ID NO: 7 - protein, 9 aa, synthetic construct
  REGION 1..9: note = Description of Artificial Sequence: Syntheticpeptide
ARSGGLPDV

SEQ ID NO: 8 - protein, 7 aa, synthetic construct
  REGION 1..7: note = Description of Artificial Sequence: Syntheticpeptide
SGGLPDW

SEQ ID NO: 9 - protein, 9 aa, synthetic construct
  REGION 1..9: note = Description of Artificial Sequence: Syntheticpeptide
ARSGGLPDW

SEQ ID NO: 10 - protein, 10 aa, synthetic construct
  REGION 1..10: note = Description of Artificial Sequence: Syntheticpeptide
RASSSVSYMY

SEQ ID NO: 11 - protein, 7 aa, synthetic construct
  REGION 1..7: note = Description of Artificial Sequence: Syntheticpeptide
ATSNLAS

SEQ ID NO: 12 - protein, 9 aa, synthetic construct
  REGION 1..9: note = Description of Artificial Sequence: Syntheticpeptide
QQWEGNPRT

SEQ ID NO: 13 - protein, 9 aa, synthetic construct
  REGION 1..9: note = Description of Artificial Sequence: Syntheticpeptide
QQWKGNPRT

SEQ ID NO: 14 - protein, 9 aa, synthetic construct
  REGION 1..9: note = Description of Artificial Sequence: Syntheticpeptide
QQWSGNPRT

SEQ ID NO: 15 - protein, 9 aa, synthetic construct
  REGION 1..9: note = Description of Artificial Sequence: Syntheticpeptide
QQWSRNPRT

SEQ ID NO: 16-100 - skipped (85 entries)

SEQ ID NO: 101 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYMELSSLRSEDTAVYYCARSGGLPDVWGQGTTVTVSS

SEQ ID NO: 102 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTMTRDTSTSTAYLELSSLRSEDTAVYYCARSGGLPDVWGQGTTVTVSS

SEQ ID NO: 103 - protein, 116 aa, synthetic construct
  REGION 1..116: note = Description of Artificial Sequence: Syntheticpolypeptide
EVQLVQSGAEVKKPGASVKVSCKASGFDIQDTYMHWVRQRPGQGLEWIGRIDPASGHTKYDPKFQVRATITTDTSTSTAYLELSSLRSEDTAVYYCARSGGLPDVWGQGTTVTVSS

SEQ ID NO: 104 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 105 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRATITRDTSTSTAYLELSSLRSEDTAVYYCARSGGLPDVWGQGTTVTVSS

SEQ ID NO: 106 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIDPASGHTKYDPKFQVRVTITTDTSTSTVYMELSSLRSEDTAVYYCARSGGLPDVWGQGTTVTVSS

SEQ ID NO: 107 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWIGRIDPASGHTKYDPKFQVRATITTDTSTSTAYLELSSLRSEDTAVYYCARSGGLPDVWGQGTTVTVSS

SEQ ID NO: 108 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 109 - protein, 116 aa, synthetic construct
  REGION 1..116: note = Description of Artificial Sequence: Syntheticpolypeptide
EVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 110 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIDPASGHTKYDPKFQVRVTITTDTSTSTAYLELSSLRSEDTAVYYCARSGGLPDVWGQGTTVTVSS

SEQ ID NO: 111 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIDPASGHTKYDPKFQVRVTITRDTSTSTAYLELSSLRSEDTAVYYCARSGGLPDVWGQGTTVTVSS

SEQ ID NO: 112 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWIGRIDPASGHTKYDPKFQVRVTITRDTSTSTAYLELSSLRSEDTAVYYCARSGGLPDVWGQGTTVTVSS

SEQ ID NO: 113 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWIGRIDPASGHTKYDPKFQVRATITRDTSTSTAYLELSSLRSEDTAVYYCARSGGLPDVWGQGTTVTVSS

SEQ ID NO: 114 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTAYLELSSLRSEDTAVYYCARSGGLPDVWGQGTTVTVSS

SEQ ID NO: 115 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRVTITTDTSTSTAYLELSSLRSEDTAVYYCARSGGLPDVWGQGTTVTVSS

SEQ ID NO: 116 - protein, 116 aa, synthetic construct
  REGION 1..116: note = Description of Artificial Sequence: Syntheticpolypeptide
EVQLVQSGAEVKKPGASVKVSCKASGFDIQDTYMHWVKQAPGQGLEWMGRIEPASGHIKYDPKFQGRVTMTRDTSTSTVYMELSSLRSEDTAVYYCARSGGLPDWWGQGTTVTVSS

SEQ ID NO: 117 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIEPASGHIKYDPKFQGRVTMTRDTSTSTVYMELSSLRSEDTAVYYCARSGGLPDWWGQGTTVTVSS

SEQ ID NO: 118 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITTDTSTSTAYLELSSLRSEDTAVYYCARSGGLPDVWGQGTTVTVSS

SEQ ID NO: 119 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTAYMELSSLRSEDTAVYYCARSGGLPDVWGQGTTVTVSS

SEQ ID NO: 120 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRVTITRDTSTSTAYLELSSLRSEDTAVYYCARSGGLPDVWGQGTTVTVSS

SEQ ID NO: 121 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQVRVTMTRDTSTSTVYMELSSLRSEDTAVYYCARSGGLPDWWGQGTTVTVSS

SEQ ID NO: 122 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQVRATMTRDTSTSTVYMELSSLRSEDTAVYYCARSGGLPDWWGQGTTVTVSS

SEQ ID NO: 123 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQVRVTITRDTSTSTVYMELSSLRSEDTAVYYCARSGGLPDWWGQGTTVTVSS

SEQ ID NO: 124 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQVRATITRDTSTSTVYMELSSLRSEDTAVYYCARSGGLPDWWGQGTTVTVSS

SEQ ID NO: 125 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQAPGQGLEWMGRIEPASGHIKYDPKFQGRVTMTRDTSTSTVYMELSSLRSEDTAVYYCARSGGLPDWWGQGTTVTVSS

SEQ ID NO: 126 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQAPGQGLEWMGRIDPASGHIKYDPKFQGRVTMTRDTSTSTVYMELSSLRSEDTAVYYCARSGGLPDWWGQGTTVTVSS

SEQ ID NO: 127 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQAPGQGLEWMGRIEPASGHIKYDPKFQGRATMTRDTSTSTVYMELSSLRSEDTAVYYCARSGGLPDWWGQGTTVTVSS

SEQ ID NO: 128 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRATMTRDTSTSTVYMELSSLRSEDTAVYYCARSGGLPDWWGQGTTVTVSS

SEQ ID NO: 129 - protein, 116 aa, synthetic construct
  REGION 1..116: note = Description of Artificial Sequence: Syntheticpolypeptide
EVQLVQSGAEVKKPGASVKVSCKASGFDIQDTYMHWVRQAPGQGLEWMGRIEPASGHIKYDPKFQGRATMTRDTSTSTVYMELSSLRSEDTAVYYCARSGGLPDWWGQGTTVTVSS

SEQ ID NO: 130 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHIKYDPKFQGRATMTRDTSTSTVYMELSSLRSEDTAVYYCARSGGLPDWWGQGTTVTVSS

SEQ ID NO: 131 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRATITTDTSTSTVYMELSSLRSEDTAVYYCARSGGLPDWWGQGTTVTVSS

SEQ ID NO: 132 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRATMTRDTSTSTAYMELSSLRSEDTAVYYCARSGGLPDWWGQGTTVTVSS

SEQ ID NO: 133 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTMTRDTSTSTAYMELSSLRSEDTAVYYCARSGGLPDWWGQGTTVTVSS

SEQ ID NO: 134 - protein, 116 aa, synthetic construct
  REGION 1..116: note = Description of Artificial Sequence: Syntheticpolypeptide
EVQLVQSGAEVKKPGASVKVSCKASGFDIQDTYMHWVRQAPGQGLEWMGRIEPASGHIKYDPKFQGRVTMTRDTSTSTAYMELSSLRSEDTAVYYCARSGGLPDWWGQGTTVTVSS

SEQ ID NO: 135 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHIKYDPKFQGRVTMTRDTSTSTAYMELSSLRSEDTAVYYCARSGGLPDWWGQGTTVTVSS

SEQ ID NO: 136 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 137 - protein, 116 aa, synthetic construct
  REGION 1..116: note = Description of Artificial Sequence: Syntheticpolypeptide
DVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 138 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQAPGQGLEWIGRIDPASGHTKYDPKFQVRATITTDTSTSTAYLELSSLRSEDTAVYYCARSGGLPDVWGQGTTVTVSS

SEQ ID NO: 139 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWIGRIDPASGHTKYDPKFQVRATITTDTSTSTAYLELSSLRSEDTAVYYCARSGGLPDVWGQGTTVTVSS

SEQ ID NO: 140 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRATITTDTSTSTAYLELSSLRSEDTAVYYCARSGGLPDVWGQGTTVTVSS

SEQ ID NO: 141 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRATITTDTSTSTVYLELSSLRSEDTAVYYCARSGGLPDVWGQGTTVTVSS

SEQ ID NO: 142 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRATITTDTSTSTAYMELSSLRSEDTAVYYCARSGGLPDVWGQGTTVTVSS

SEQ ID NO: 143 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRATITTDTSTSTVYMELSSLRSEDTAVYYCARSGGLPDVWGQGTTVTVSS

SEQ ID NO: 144 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRATMTTDTSTSTAYLELSSLRSEDTAVYYCARSGGLPDVWGQGTTVTVSS

SEQ ID NO: 145 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRATITRDTSTSTAYLELSSLRSEDTAVYYCARSGGLPDVWGQGTTVTVSS

SEQ ID NO: 146 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRVTMTTDTSTSTAYLELSSLRSEDTAVYYCARSGGLPDVWGQGTTVTVSS

SEQ ID NO: 147 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRATMTRDTSTSTAYLELSSLRSEDTAVYYCARSGGLPDVWGQGTTVTVSS

SEQ ID NO: 148 - protein, 116 aa, synthetic construct
  REGION 1..116: note = Description of Artificial Sequence: Syntheticpolypeptide
QVQLVQSGAEVKKPGASVKVSCKASGFDIQDTYMHWVKQRPGQGLEWIGRIDPASGHTKYDPKFQVRVTMTRDTSTSTAYLELSSLRSEDTAVYYCARSGGLPDVWGQGTTVTVSS

SEQ ID NO: 149 - protein, 116 aa, synthetic construct
  REGION 1..116: note = Description of Artificial Sequence: Syntheticpolypeptide
DVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 150 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIEPASGHIKYDPKFQGRVTMTRDTSTSTVYMELSSLRSEDTAVYYCARSGGLPDWWGQGTTVTVSS

SEQ ID NO: 151 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHIKYDPKFQGRVTMTRDTSTSTVYMELSSLRSEDTAVYYCARSGGLPDWWGQGTTVTVSS

SEQ ID NO: 152 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWIGRIEPASGHIKYDPKFQGRVTMTRDTSTSTVYMELSSLRSEDTAVYYCARSGGLPDWWGQGTTVTVSS

SEQ ID NO: 153 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWIGRIDPASGHIKYDPKFQGRVTMTRDTSTSTVYMELSSLRSEDTAVYYCARSGGLPDWWGQGTTVTVSS

SEQ ID NO: 154 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHVKYDPKFQGRVTMTRDTSTSTVYMELSSLRSEDTAVYYCARSGGLPDWWGQGTTVTVSS

SEQ ID NO: 155 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTITRDTSTSTVYMELSSLRSEDTAVYYCARSGGLPDWWGQGTTVTVSS

SEQ ID NO: 156 - protein, 116 aa, synthetic construct
  REGION 1..116: note = Description of Artificial Sequence: Syntheticpolypeptide
EVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 157 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTMTTDTSTSTVYMELSSLRSEDTAVYYCARSGGLPDWWGQGTTVTVSS

SEQ ID NO: 158 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTITTDTSTSTVYMELSSLRSEDTAVYYCARSGGLPDWWGQGTTVTVSS

SEQ ID NO: 159 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHTKYDPKFQGRVTMTRDTSTSTVYMELSSLRSEDTAVYYCARSGGLPDWWGQGTTVTVSS

SEQ ID NO: 160 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRATITRDTSTSTVYMELSSLRSEDTAVYYCARSGGLPDWWGQGTTVTVSS

SEQ ID NO: 161 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTMTRDTSTSTVYLELSSLRSEDTAVYYCARSGGLPDWWGQGTTVTVSS

SEQ ID NO: 162 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIEPASGHIKYDPKFQGRVTMTRDTSTSTAYLELSSLRSEDTAVYYCARSGGLPDWWGQGTTVTVSS

SEQ ID NO: 163 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIDPASGHTKYDPKFQVRATITTDTSTSTAYLELSSLRSEDTAVYYCARSGGLPDVWGQGTTVTVSS

SEQ ID NO: 164 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWIGRIDPASGHTKYDPKFQVRVTITTDTSTSTAYLELSSLRSEDTAVYYCARSGGLPDVWGQGTTVTVSS

SEQ ID NO: 165 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIDPASGHTKYDPKFQVRVTITTDTSTSTVYLELSSLRSEDTAVYYCARSGGLPDVWGQGTTVTVSS

SEQ ID NO: 166 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIDPASGHTKYDPKFQVRVTITTDTSTSTAYMELSSLRSEDTAVYYCARSGGLPDVWGQGTTVTVSS

SEQ ID NO: 167 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQRPGQGLEWMGRIDPASGHTKYDPKFQVRVTMTTDTSTSTAYLELSSLRSEDTAVYYCARSGGLPDVWGQGTTVTVSS

SEQ ID NO: 168 - protein, 116 aa, synthetic construct
  REGION 1..116: note = Description of Artificial Sequence: Syntheticpolypeptide
QVQLVQSGAEVKKPGASVKVSCKASGFDIQDTYMHWVRQAPGQGLEWMGRIDPASGHIKYDPKFQGRATITTDTSTSTVYMELSSLRSEDTAVYYCARSGGLPDWWGQGTTVTVSS

SEQ ID NO: 169 - protein, 116 aa, synthetic construct
  REGION 1..116: note = Description of Artificial Sequence: Syntheticpolypeptide
EVQLVQSGAEVKKPGASVKVSCKASGFDIQDTYMHWVRQAPGQGLEWMGRIEPASGHIKYDPKFQGRATITTDTSTSTVYMELSSLRSEDTAVYYCARSGGLPDWWGQGTTVTVSS

SEQ ID NO: 170-200 - skipped (31 entries)

SEQ ID NO: 201 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 202 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 203 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 204 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWKGNPRTFGGGTKLEIK

SEQ ID NO: 205 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRPWIYATSNLASGIPDRFSGSGSGTDFTLTISRLEPEDFAVYYCQQWEGNPRTFGGGTKLEIK

SEQ ID NO: 206 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRPLIYATSNLASGIPDRFSGSGSGTDFTLTISRLEPEDFAVYYCQQWKGNPRTFGGGTKLEIK

SEQ ID NO: 207 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSRNPRTFGGGTKLEIK

SEQ ID NO: 208 - protein, 106 aa, synthetic construct
  REGION 1..106: note = Description of Artificial Sequence: Syntheticpolypeptide
EIVLTQSPGTLSASPGERATMSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 209 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGVPDRFSGSGSGTDFTLTISRLEPEDFAVYYCQQWEGNPRTFGGGTKLEIK

SEQ ID NO: 210 - protein, 106 aa, synthetic construct
  REGION 1..106: note = Description of Artificial Sequence: Syntheticpolypeptide
EIVLTQSPGTLSASPGERATLSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 211 - protein, 106 aa, synthetic construct
  REGION 1..106: note = Description of Artificial Sequence: Syntheticpolypeptide
EIVLTQSPGTLSASPGERATMSCRASSSVSYMYWYQQKPGQAPRLLIYATSNLASGVPDRFSGSGSGTDYTLTISRVEPEDFAVYYCQQWSGNPRTFGGGTKLEIK

SEQ ID NO: 212 - protein, 106 aa, synthetic construct
  REGION 1..106: note = Description of Artificial Sequence: Syntheticpolypeptide
EIVLTQSPGTLSASPGERATMSCRASSSVSYMYWYQQKPGQAPRPWIYATSNLASGVPDRFSGSGSGTDFTLTISRVEPEDFAVYYCQQWSGNPRTFGGGTKLEIK

SEQ ID NO: 213 - protein, 106 aa, synthetic construct
  REGION 1..106: note = Description of Artificial Sequence: Syntheticpolypeptide
EIVLTQSPGTLSASPGERATMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 214 - protein, 106 aa, synthetic construct
  REGION 1..106: note = Description of Artificial Sequence: Syntheticpolypeptide
EIVLTQSPGTLSASPGERATMSCRASSSVSYMYWYQQKPGQAPRPWIYATSNLASGVPDRFSGSGSGTDFTLTISRLEPEDFAVYYCQQWSGNPRTFGGGTKLEIK

SEQ ID NO: 215 - protein, 106 aa, synthetic construct
  REGION 1..106: note = Description of Artificial Sequence: Syntheticpolypeptide
EIVLTQSPGTLSASPGERATMSCRASSSVSYMYWYQQKPGQAPRPWIYATSNLASGIPDRFSGSGSGTDYTLTISRVEPEDFAVYYCQQWSGNPRTFGGGTKLEIK

SEQ ID NO: 216 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDYTLTISRLEPEDFAVYYCQQWEGNPRTFGGGTKLEIK

SEQ ID NO: 217 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDFTLTISRVEPEDFAVYYCQQWEGNPRTFGGGTKLEIK

SEQ ID NO: 218 - protein, 106 aa, synthetic construct
  REGION 1..106: note = Description of Artificial Sequence: Syntheticpolypeptide
EIVLTQSPGTLSLSPGERATLSCRASSSVSYMYWYQQKPGQAPRLLIYATSNLASGIPDRFSGSGSGTDYTLTISRVEPEDFAVYYCQQWEGNPRTFGGGTKLEIK

SEQ ID NO: 219 - protein, 106 aa, synthetic construct
  REGION 1..106: note = Description of Artificial Sequence: Syntheticpolypeptide
EIVLTQSPGTLSASPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 220 - protein, 106 aa, synthetic construct
  REGION 1..106: note = Description of Artificial Sequence: Syntheticpolypeptide
EIVLTQSPGTMSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 221-300 - skipped (80 entries)

SEQ ID NO: 301 - protein, 118 aa, synthetic construct
  REGION 1..118: note = Description of Artificial Sequence: Syntheticpolypeptide
  VARIANT 1: note = Any amino acid
  VARIANT 38: note = Any amino acid
  VARIANT 40: note = Any amino acid
  VARIANT 48: note = Any amino acid
  VARIANT 50..66: note = This region may encompass one of the following sequences: RIDPASGHTKYDPKFQV or RIEPASGHIKYDPKFQG or RIDPASGHIKYDPKFQG or RIEPASGHIKYDPKFQV
  VARIANT 68: note = Any amino acid
  VARIANT 70: note = Any amino acid
  VARIANT 72: note = Any amino acid
  VARIANT 79: note = Any amino acid
  VARIANT 81: note = Any amino acid
  VARIANT 99..107: note = This region may encompass one of the following sequences:SGGLPDV or ARSGGLPDV or SGGLPDW or ARSGGLPDW
  REGION 1..118: note = See specification as filed for detailed description ofsubstitutions and preferred embodiments
XVQLVQSGAEVKKPGASVKVSCKASGFDIQDTYMHWVXQXPGQGLEWXGXXXXXXXXXXXXXXXXXRXTXTXDTSTSTXYXELSSLRSEDTAVYYCARXXXXXXXXXWGQGTTVTVSS

SEQ ID NO: 302 - protein, 116 aa, synthetic construct
  REGION 1..116: note = Description of Artificial Sequence: Syntheticpolypeptide
  VARIANT 1: note = Any amino acid
  VARIANT 38: note = Any amino acid
  VARIANT 40: note = Any amino acid
  VARIANT 48: note = Any amino acid
  VARIANT 50..66: note = This region may encompass one of the following sequences:RIDPASGHTKYDPKFQV or RIEPASGHIKYDPKFQG orRIDPASGHIKYDPKFQG or RIEPASGHIKYDPKFQV
  VARIANT 68: note = Any amino acid
  VARIANT 70: note = Any amino acid
  VARIANT 72: note = Any amino acid
  VARIANT 79: note = Any amino acid
  VARIANT 81: note = Any amino acid
  VARIANT 97..105: note = This region may encompass one of the following sequences:SGGLPDV or ARSGGLPDV or SGGLPDW or ARSGGLPDW
  REGION 1..116: note = See specification as filed for detailed description ofsubstitutions and preferred embodiments
XVQLVQSGAEVKKPGASVKVSCKASGFDIQDTYMHWVXQXPGQGLEWXGXXXXXXXXXXXXXXXXXRXTXTXDTSTSTXYXELSSLRSEDTAVYYCXXXXXXXXXWGQGTTVTVSS

SEQ ID NO: 303 - protein, 106 aa, synthetic construct
  REGION 1..106: note = Description of Artificial Sequence: Syntheticpolypeptide
  VARIANT 45..46: note = Any amino acid
  VARIANT 88..96: note = This region may encompass one of the following sequences:QQWEGNPRT or QQWKGNPRT or QQWSGNPRT or QQWSRNPRT
  REGION 1..106: note = See specification as filed for detailed description ofsubstitutions and preferred embodiments
EIVLTQSPGTLSLSPGERATLSCRASSSVSYMYWYQQKPGQAPRXXIYATSNLASGIPDRFSGSGSGTDFTLTISRLEPEDFAVYYCXXXXXXXXXFGGGTKLEIK

SEQ ID NO: 304 - protein, 25 aa, synthetic construct [sequence omitted]

SEQ ID NO: 305 - protein, 14 aa, synthetic construct
  REGION 1..14: note = Description of Artificial Sequence: Syntheticpeptide
WVKQRPGQGLEWMG

SEQ ID NO: 306 - protein, 32 aa, synthetic construct
  REGION 1..32: note = Description of Artificial Sequence: Syntheticpolypeptide
RVTITRDTSTSTVYLELSSLRSEDTAVYYCAR

SEQ ID NO: 307 - protein, 30 aa, synthetic construct
  REGION 1..30: note = Description of Artificial Sequence: Syntheticpolypeptide
RVTITRDTSTSTVYLELSSLRSEDTAVYYC

SEQ ID NO: 308 - protein, 11 aa, synthetic construct
  REGION 1..11: note = Description of Artificial Sequence: Syntheticpeptide
WGQGTTVTVSS

SEQ ID NO: 309 - protein, 23 aa, synthetic construct
  REGION 1..23: note = Description of Artificial Sequence: Syntheticpeptide
EIVLTQSPGTLSLSPGERATLSC

SEQ ID NO: 310 - protein, 15 aa, synthetic construct
  REGION 1..15: note = Description of Artificial Sequence: Syntheticpeptide
WYQQKPGQAPRPLIY

SEQ ID NO: 311 - protein, 32 aa, synthetic construct
  REGION 1..32: note = Description of Artificial Sequence: Syntheticpolypeptide
GIPDRFSGSGSGTDFTLTISRLEPEDFAVYYC

SEQ ID NO: 312 - protein, 10 aa, synthetic construct
  REGION 1..10: note = Description of Artificial Sequence: Syntheticpeptide
FGGGTKLEIK

SEQ ID NO: 313 - protein, 14 aa, synthetic construct
  REGION 1..14: note = Description of Artificial Sequence: Syntheticpeptide
WVRQRPGQGLEWIG

SEQ ID NO: 314 - protein, 32 aa, synthetic construct
  REGION 1..32: note = Description of Artificial Sequence: Syntheticpolypeptide
RATITTDTSTSTAYLELSSLRSEDTAVYYCAR

SEQ ID NO: 315 - protein, 30 aa, synthetic construct
  REGION 1..30: note = Description of Artificial Sequence: Syntheticpolypeptide
RATITTDTSTSTAYLELSSLRSEDTAVYYC

SEQ ID NO: 316 - protein, 98 aa, Homo sapiens
QVQLVQSGAEVKKPGASVKVSCKASGYTFNSYYMHWVRQAPGQGLEWMGIINPSGGSTSYAQKFQGRVTMTRDTSTSTVYMELSSLRSEDTAVYYCAR

SEQ ID NO: 317 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 318 - skipped

SEQ ID NO: 319 - protein, 107 aa, Homo sapiens [sequence omitted]

SEQ ID NO: 320 - protein, 330 aa, Homo sapiens [sequence omitted]

SEQ ID NO: 321 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 331 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 332 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 340 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 341 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 343 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 344 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 356 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEEFGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 327 aa, synthetic construct
  REGION 1..327: note = Description of Artificial Sequence: Syntheticpolypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 366 - protein, 327 aa, synthetic construct
  REGION 1..327: note = Description of Artificial Sequence: Syntheticpolypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 367 - protein, 327 aa, synthetic construct
  REGION 1..327: note = Description of Artificial Sequence: Syntheticpolypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 368 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEEFGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 446 aa, synthetic construct
  REGION 1..446: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 378 - protein, 443 aa, synthetic construct
  REGION 1..443: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 379 - protein, 443 aa, synthetic construct
  REGION 1..443: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 380 - protein, 443 aa, synthetic construct
  REGION 1..443: note = Description of Artificial Sequence: Syntheticpolypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 381 - protein, 213 aa, synthetic construct
  REGION 1..213: note = Description of Artificial Sequence: Syntheticpolypeptide
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 382 - protein, 214 aa, synthetic construct
  REGION 1..214: note = Description of Artificial Sequence: Syntheticpolypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 383 - protein, 453 aa, synthetic construct
  REGION 1..453: note = Description of Artificial Sequence: Syntheticpolypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 384-390 - skipped (7 entries)

SEQ ID NO: 391 - protein, 6 aa, synthetic construct
  REGION 1..6: note = Description of Artificial Sequence: Syntheticpeptide
HHHHHH

SEQ ID NO: 392-400 - skipped (9 entries)

SEQ ID NO: 401 - protein, 10 aa, unidentified
  VARIANT 3: note = may be P, S, D, Q, or N
  VARIANT 5: note = may be T or R
  VARIANT 6: note = may be N, T, Y, or H
GYXFXXYGIS

SEQ ID NO: 402 - protein, 17 aa, synthetic construct
  VARIANT 8: note = may be N, G, V, K, or A
  VARIANT 9: note = may be T or K
  VARIANT 10: note = may be H or N
  VARIANT 13: note = may be Q or R
  VARIANT 14: note = may be K or M
  VARIANT 15: note = may be L or H
  VARIANT 4: note = may be T, P, S, or A
WISXYNGXXXYAXXXQG

SEQ ID NO: 403 - protein, 15 aa, synthetic construct
  VARIANT 9: note = may be Y or F
  VARIANT 15: note = may be V, A, or G
  VARIANT 8: note = may be S or A
ENYYGSGXXRGGMDX

SEQ ID NO: 404 - protein, 11 aa, synthetic construct
RASQSVSSYLA

SEQ ID NO: 405 - protein, 7 aa, synthetic construct
DASNRAT

SEQ ID NO: 406 - protein, 9 aa, synthetic construct
QQRSNWPWT

SEQ ID NO: 407 - protein, 10 aa, synthetic construct
GYDFTYYGIS

SEQ ID NO: 408 - protein, 17 aa, synthetic construct
WISTYNGNTHYARMLQG

SEQ ID NO: 409 - protein, 15 aa, synthetic construct
ENYYGSGAYRGGMDV

SEQ ID NO: 410 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 411 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 412 - protein, 453 aa, synthetic construct; identical to SEQ ID NO: 383

SEQ ID NO: 413 - protein, 214 aa, synthetic construct; identical to SEQ ID NO: 382

SEQ ID NO: 414 - protein, 10 aa, synthetic construct
GYTFTSYDIN

SEQ ID NO: 415 - protein, 10 aa, synthetic construct
WLNPNSGYTG

SEQ ID NO: 416 - protein, 10 aa, synthetic construct
EVPETAAFEY

SEQ ID NO: 417 - protein, 14 aa, synthetic construct
TSSSSDIGAGLGVH

SEQ ID NO: 418 - protein, 7 aa, synthetic construct
GYYNRPS

SEQ ID NO: 419 - protein, 10 aa, synthetic construct
QSWDGTLSAL

SEQ ID NO: 420 - protein, 119 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 421 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 422 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 423 - protein, 216 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 424 - protein, 457 aa, synthetic construct
DVDPASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNRGPIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 425 - protein, 174 aa, synthetic construct
GGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLLGFYEHGDGCVSCPTSTLGSCPERCAAVCGWRQ

SEQ ID NO: 426 - protein, 5 aa, synthetic construct
TYWLG

SEQ ID NO: 427 - protein, 16 aa, synthetic construct
IMSPVDSDIRYSPSFQ

SEQ ID NO: 428 - protein, 10 aa, synthetic construct
RRPGQGYFDF

SEQ ID NO: 429 - protein, 11 aa, synthetic construct
RASQGISSWLA

SEQ ID NO: 430 - protein, 7 aa, synthetic construct
AASSLQS

SEQ ID NO: 431 - protein, 9 aa, synthetic construct
QQYNIYPYT

SEQ ID NO: 432 - protein, 5 aa, synthetic construct
NYWIG

SEQ ID NO: 433 - protein, 17 aa, synthetic construct
IIDPSNSYTRYSPSFQG

SEQ ID NO: 434 - protein, 8 aa, synthetic construct
WYYKPFDV

SEQ ID NO: 435 - protein, 14 aa, synthetic construct
TGSSSNIGSGYDVH

SEQ ID NO: 436 - protein, 7 aa, synthetic construct
GNSKRPS

SEQ ID NO: 437 - protein, 11 aa, synthetic construct
ASWTDGLSLVV

SEQ ID NO: 438 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 439 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 440 - protein, 11 aa, synthetic construct
KASRDVAIAVA

SEQ ID NO: 441 - protein, 7 aa, synthetic construct
WASTRHT

SEQ ID NO: 442 - protein, 9 aa, synthetic construct
HQYSSYPFT

SEQ ID NO: 443 - protein, 10 aa, synthetic construct
GYTFTDQTIH

SEQ ID NO: 444 - protein, 17 aa, synthetic construct
YIYPRDDSPKYNENFKG

SEQ ID NO: 445 - protein, 11 aa, synthetic construct
PDRSGYAWFIY

SEQ ID NO: 446 - protein, 10 aa, synthetic construct
GNTFTDQTIH

SEQ ID NO: 447 - protein, 10 aa, synthetic construct
GFTFTDQTIH

SEQ ID NO: 448 - protein, 10 aa, synthetic construct
GGTFTDQTIH

SEQ ID NO: 449 - protein, 107 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 450 - protein, 120 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 451 - protein, 120 aa, synthetic construct
QVQLVQSGAEVKKPGSSVKVSCKASGFTFTDQTIHWVRQAPGQGLEWMGYIYPRDDSPKYNENFKGKVTLTADKSTSTAYMELSSLRSEDTAVYYCAIPDRSGYAWFIYWGQGTLVTVSS

SEQ ID NO: 452 - protein, 107 aa, synthetic construct
DIQMTQSPSSLSASVGDRVTITCKASRDVAIAVAWYQQKPGKVPKLLLFWASTRHTGVPDRFSGSGSGTDFTLTISSLQPEDLADYYCHQYSSYPFTFGQGTKLEIK

SEQ ID NO: 453 - protein, 214 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 454 - protein, 449 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 455 - protein, 5 aa, synthetic construct
SYGMH

SEQ ID NO: 456 - protein, 18 aa, synthetic construct
VIWYDGSNEYYADSVKGR

SEQ ID NO: 457 - protein, 15 aa, synthetic construct
DRGYTSSWYPDAFDI

SEQ ID NO: 458 - protein, 14 aa, synthetic construct
TGSSSNTGAGYDVH

SEQ ID NO: 459 - protein, 7 aa, synthetic construct
GSGNRPS

SEQ ID NO: 460 - protein, 11 aa, synthetic construct
QSYDSSLSGWV

SEQ ID NO: 461 - protein, 124 aa, synthetic construct
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 462 - protein, 111 aa, synthetic construct
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVL

SEQ ID NO: 463 - protein, 11 aa, synthetic construct
KASDHILKFLT

SEQ ID NO: 464 - protein, 7 aa, synthetic construct
GATSLET

SEQ ID NO: 465 - protein, 9 aa, synthetic construct
QMYWSTPFT

SEQ ID NO: 466 - protein, 10 aa, synthetic construct
GYKFTRYVMH

SEQ ID NO: 467 - protein, 17 aa, synthetic construct
YINPYNDGTNYNEKFKG

SEQ ID NO: 468 - protein, 8 aa, synthetic construct
ARNWDTGL

SEQ ID NO: 469 - protein, 115 aa, synthetic construct
QVQLVQSGAEVKKPGSSVKVSCKASGYKFTRYVMHWVRQAPGQGLEWMGYINPYNDGTNYNEKFKGRVTITADKSTSTAYMELSSLRSEDTAVYYCARNWDTGLWGQGTTVTVSS

SEQ ID NO: 470 - protein, 107 aa, synthetic construct
DIQMTQSPSSLSASVGDRVTITCKASDHILKFLTWYQQKPGKAPKLLIYGATSLETGVPSRFSGSGSGTDFTLTISSLQPEDFATYYCQMYWSTPFTFGGGTKVEIK

SEQ ID NO: 471 - protein, 441 aa, synthetic construct
QVQLVQSGAEVKKPGSSVKVSCKASGYKFTRYVMHWVRQAPGQGLEWMGYINPYNDGTNYNEKFKGRVTITADKSTSTAYMELSSLRSEDTAVYYCARNWDTGLWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 472 - protein, 214 aa, synthetic construct
DIQMTQSPSSLSASVGDRVTITCKASDHILKFLTWYQQKPGKAPKLLIYGATSLETGVPSRFSGSGSGTDFTLTISSLQPEDFATYYCQMYWSTPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 473 - protein, 11 aa, synthetic construct
RTSENIYSYLA

SEQ ID NO: 474 - protein, 7 aa, synthetic construct
NAKTLAE

SEQ ID NO: 475 - protein, 9 aa, synthetic construct
QHHYGIPFT

SEQ ID NO: 476 - protein, 10 aa, synthetic construct
GYIFITYWMT

SEQ ID NO: 477 - protein, 17 aa, synthetic construct
QIFPASGSADYNEKFEG

SEQ ID NO: 478 - protein, 7 aa, synthetic construct
GGGGFAY

SEQ ID NO: 479 - protein, 214 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 480 - protein, 446 aa, synthetic construct
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 481 - protein, 170 aa, synthetic construct
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 482 - protein, 13 aa, synthetic construct
SGSRSNIGSNTVK

SEQ ID NO: 483 - protein, 7 aa, synthetic construct
YNDQRPS

SEQ ID NO: 484 - protein, 12 aa, synthetic construct
QSYDRYTHPALL

SEQ ID NO: 485 - protein, 9 aa, synthetic construct
FTFSSYGMH

SEQ ID NO: 486 - protein, 17 aa, synthetic construct
FIRYDGSNKYYADSVKG

SEQ ID NO: 487 - protein, 6 aa, synthetic construct
HGSHDN

SEQ ID NO: 488 - protein, 115 aa, synthetic construct [sequence omitted]

SEQ ID NO: 489 - protein, 112 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

SEQ ID NO: 490 - protein, 157 aa, synthetic construct
GGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLLGFYEHGDGCVSCPTS

SEQ ID NO: 491 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 492 - protein, 108 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

(19) United States
(12) Patent Application Publication
(10) Pub. No.: US 2025/0243289 A1
(43) Pub. Date: Jul. 31, 2025

(54) METHODS OF TREATING INFLAMMATORY DISEASES WITH COMBINATION OF TL1A INHIBITORS AND IL23 INHIBITORS

(71) Applicant: Prometheus Biosciences, Inc., San Diego, CA (US)

(72) Inventors: Ernesto J. Munoz, Rancho Sante Fe, CA (US); Heather Llewellyn, San Diego, CA (US); Burton Barnett, Poway, CA (US)

(73) Assignee: Prometheus Biosciences, Inc., San Diego, CA (US)

(21) Appl. No.: 18/724,826

(22) PCT Filed: Jan. 6, 2023
(86) PCT No.: PCT/US2023/060273

§ 371 Date: Jun. 27, 2024

Related U.S. Application Data
(60) Provisional application No. 63/297,654, filed on Jan. 7, 2022.

(51) Int. Cl. C07K 16/28; A61K 39/00; A61P 1/00; C07K 16/24

(52) U.S. Cl. CPC C07K 16/2875; A61P 1/00; C07K 16/244; A61K 2039/507; A61K 2039/545; C07K 2317/565; C07K 2317/567; C07K 2317/92

(57) ABSTRACT

Described herein are methods of treating inflammatory diseases or conditions such as inflammatory bowel diseases including Crohn's disease and ulcerative colitis, the methods comprising administering a TL1A inhibitor and an IL23 inhibitor contemporaneously for the duration of treatment or contemporaneously during an induction phase of treatment followed by maintenance of response with the TL1A inhibitor alone or IL23 inhibitor alone.

### 1. CROSS-REFERENCE TO RELATED APPLICATIONS

This application claims benefit of U.S. Provisional Patent Application No. 63/297,654, filed on Jan. 7, 2022, which is incorporated herein by reference in its entirety.

### 2. REFERENCE TO A SEQUENCE LISTING

This application contains a Sequence Listing which has been submitted electronically in XML format and is hereby incorporated by reference in its entirety. Said XML copy, created on Dec. 12, 2024, is named 25752-US-PCT_SL.xml and is 414,177 bytes in size.

### 3. BACKGROUND

Inflammatory diseases or conditions occur when the immune system attacks the body's own tissues, resulting in inflammation. Such inflammation diseases or conditions can be caused by a multitude of factors, including infections or other foreign subjects in the body, injuries or tissue damages in the body, or genetic factors, among others. If left unattended to, inflammatory diseases or conditions can cause severe illness and significantly impact the patient's life. The inflammation diseases or conditions are characterized by inflammation and include inflammatory bowel disease (IBD).

IBD refers to a collection of intestinal disorders causing inflammatory conditions in the gastrointestinal tract. Severe forms of IBD may be characterized by intestinal fibrosis, which is the accumulation of scar tissue in the intestinal wall. The primary types of IBD are ulcerative colitis (UC) and Crohn's Disease (CD). Both UC and CD are chronic, relapsing, remitting, inflammatory conditions of the gastrointestinal tract that begin most commonly during adolescence and young adulthood. UC involves the mucosal layer of the large intestine, and symptoms include abdominal pain and diarrhea, frequently with blood and mucus. CD can affect the entire thickness of the bowel wall and all parts of the GI tract from mouth to anus. CD symptoms include abdominal pain, diarrhea, and other more insidious symptoms such as weight loss, nutritional deficiencies, and fever. The prevalence of IBD globally is approximately 5 million and the disease affects over 2 million people in the US.

The current standard of care for the treatment of patients with moderate to severe IBD are generally immunomodulatory agents that are anti-inflammatory. None of these therapies address fibrosis in IBD. Since the approval of the first anti-TNF agent for the treatment of CD in 1998, the availability of newer biological agents, including anti-integrin, Janus kinase (JAK) inhibitors, and anti-IL12/23 has improved the care of moderate to severe UC and CD (JAK inhibitors in UC only). However, none of these subsequently approved therapies have demonstrated significant improvement in effect size relative to anti-TNF. Moreover, among those patients who do respond, up to 45% will lose response over time. Current therapies used in the treatment of UC and CD apply a one-size-fits-all approach without regard to genetic or biologic variations in the patient. Existing approaches continue to leave unmet patient need.

The inventors therefore recognize that the heterogeneity of disease pathogenesis and clinical course, combined with the variable response to treatment and its associated side effects, are inadequately addressed by the therapeutic approach covering only a small subset of the spectrum of causes and factors for IBD. Accordingly, there is a need for therapeutics to cover a wider spectrum of the IBD heterogeneity.

### 4. SUMMARY

In one aspect, provided herein is a method of treating an inflammatory disease or condition in a subject comprising administering to the subject a first composition comprising a first therapeutically effective amount of an inhibitor of tumor necrosis factor-like protein 1A (“TL1A” and such inhibitor, “TL1A inhibitor”) and administering to the subject a second composition comprising a second therapeutically effective amount of an inhibitor of interleukin 23 (“IL23” and such inhibitor, “IL23 inhibitor”).

In one aspect, provided herein is a method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor.

In one aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor.

In some embodiments, the maintenance regimen comprises a third therapeutically effective amount of the TL1A inhibitor. In some embodiments, the maintenance regimen comprises a fourth therapeutically effective amount of the IL23 inhibitor. In some embodiments, the third therapeutically effective amount is identical to the first therapeutically effective amount or the third therapeutically effective amount is less than the first therapeutically effective amount. In some embodiments, the fourth therapeutically effective amount is identical to the second therapeutically effective amount or the fourth therapeutically effective amount is less than the second therapeutically effective amount.

In one aspect, provided herein is a method of treating an inflammatory disease or condition in a subject comprising administering to the subject a composition comprising a first therapeutically effective amount of a TL1A inhibitor and a second therapeutically effective amount of an IL23 inhibitor.

In some embodiments, the molar ratio of the first therapeutically effective amount to the second therapeutically effective amount is about 50:1, about 40:1, about 30:1, about 20:1, about 15:1, about 12:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:12, about 1:15, about 1:20, about 1:30, about 1:40, or about 1:50.

In some embodiments, the inflammatory disease or condition is inflammatory bowel disease (IBD). In some embodiments, the inflammatory disease or condition is ulcerative colitis (UC) or indeterminate colitis. In some embodiments, the inflammatory disease or condition is moderately to severely active UC. In some embodiments, the inflammatory disease or condition is Crohn's Disease (CD).

In some embodiments, the TL1A inhibitor is an inhibitor of TL1A expression or an inhibitor of TL1A activity. In some embodiments, the TL1A inhibitor is an anti-TL1A antibody or antigen binding fragment thereof. In some embodiments, the antibody or antigen binding fragment binds to both monomeric TL1A and trimeric TL1A. In some embodiments, the TL1A inhibitor blocks interaction of TL1A to Death Receptor 3 (“DR3”). In some embodiments, the binding affinity of the antibody or antigen binding fragment to monomeric TL1A as measured by dissociation equilibrium constant (KD-monomer) is comparable to binding affinity of the antibody or antigen binding fragment to trimeric TL1A as measured by dissociation equilibrium constant (KD-trimer). In some embodiments, the KD-monomer is within 1.5, 2, 3, 4, 5, 6, 7, 8, 9, or 10 fold of the KD-trimer. In some embodiments, the KD-monomer is no more than 0.06 nM. In some embodiments, the KD-trimer is no more than 0.06 nM. In some embodiments, the anti-TL1A antibody or antigen binding fragment binds to both monomeric TL1A and trimeric TL1A, and wherein the anti-TL1A antibody or antigen binding fragment blocks interaction of TL1A to DR3.

In some embodiments, the first therapeutically effective amount is 200 mg/dose, 250 mg/dose, 300 mg/dose, 350 mg/dose, 400 mg/dose, 450 mg/dose, 500 mg/dose, 550 mg/dose, 600 mg/dose, 650 mg/dose, 700 mg/dose, 750 mg/dose, 800 mg/dose, 850 mg/dose, 900 mg/dose, 950 mg/dose, 1000 mg/dose, 1100 mg/dose, 1200 mg/dose, 1250 mg/dose, 1300 mg/dose, 1400 mg/dose, 1500 mg/dose, 1600 mg/dose, 1700 mg/dose, 1750 mg/dose, 1800 mg/dose, 1900 mg/dose, or 2000 mg/dose. In some embodiments, the first therapeutically effective amount comprises 1, 2, 3, 4, 5, 6, 7, 8, 9, 10, 11, 12, or more doses. In some embodiments, the first therapeutically effective amount comprises (i) 1000 mg/dose on week 0, 1000 mg/dose on week 2, 1000 mg/dose on week 6, and 1000 mg/dose on week 10; (ii) 500 mg/dose on week 0, 500 mg/dose on week 2, 500 mg/dose on week 6, and 500 mg/dose on week 10; (iii) 1000 mg/dose on week 0, 1000 mg/dose on week 2, 1000 mg/dose on week 6, and 500 mg/dose on week 10; (iv) 1000 mg/dose on week 0, 1000 mg/dose on week 2, 500 mg/dose on week 6, and 500 mg/dose on week 10; or (v) 1000 mg/dose on week 0, 500 mg/dose on week 2, 500 mg/dose on week 6, and 500 mg/dose on week 10. In some embodiments, the first therapeutically effective amount comprises 2000, 1950, 1900, 1850, 1800, 1750, 1700, 1650, 1600, 1550, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, or 200 mg/dose.

In some embodiments, the administering comprises administering once every 2, 4, 6, 8, 10, or 12 weeks. In some embodiments, the administering comprises administering once every 2 or 4 weeks for the first 2 administrations and then once every 2, 4, 6, or 8 weeks for the remaining administration.

In some embodiments, the first therapeutically effective amount comprises 1000 mg/dose every 4 weeks, 500 mg/dose every 4 weeks, 250 mg/dose every 4 weeks, 100 mg/dose every 4 weeks, 1000 mg/dose every 2 weeks, 500 mg/dose every 2 weeks, 250 mg/dose every 2 weeks, or 100 mg/dose every 2 weeks.

In some embodiments, at least 91%, at least 92%, at least 93%, at least 94%, at least 95%, at least 96%, at least 97%, at least 98%, or at least 99% of the monomeric TL1A in the blood of the subject is occupied by the anti-TL1A antibody or antigen binding fragment after administering the first therapeutically effective amount. In some embodiments, at least 91%, at least 92%, at least 93%, at least 94%, at least 95%, at least 96%, at least 97%, at least 98%, or at least 99% of the trimeric TL1A in the blood of the subject is occupied by the anti-TL1A antibody or antigen binding fragment after administering the first therapeutically effective amount.

In one aspect, provided herein is a pharmaceutical composition comprising a first therapeutically effective amount of an inhibitor of tumor necrosis factor-like protein 1A (“TL1A” and such inhibitor, “TL1A inhibitor”) and a second therapeutically effective amount of an inhibitor of interleukin 23 (“IL23 inhibitor”). In some embodiments, the molar ratio of the first therapeutically effective amount to the second therapeutically effective amount is about 50:1, about 40:1, about 30:1, about 20:1, about 15:1, about 12:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:12, about 1:15, about 1:20, about 1:30, about 1:40, or about 1:50. In some embodiments, the TL1A inhibitor is an inhibitor of TL1A expression or an inhibitor of TL1A activity. In some embodiments, the TL1A inhibitor is an anti-TL1A antibody or antigen binding fragment thereof. In some embodiments, the antibody or antigen binding fragment binds to both monomeric TL1A and trimeric TL1A. In some embodiments, the TL1A inhibitor blocks interaction of TL1A to Death Receptor 3 (“DR3”). In some embodiments, the binding affinity of the antibody or antigen binding fragment to monomeric TL1A as measured by dissociation equilibrium constant (KD-monomer) is comparable to binding affinity of the antibody or antigen binding fragment to trimeric TL1A as measured by dissociation equilibrium constant (KD-trimer). In some embodiments, the KD-monomer is within 1.5, 2, 3, 4, 5, 6, 7, 8, 9, or 10 fold of the KD-trimer. In some embodiments, the KD-monomer is no more than 0.06 nM. In some embodiments, the KD-trimer is no more than 0.06 nM. In some embodiments, the antibody or antigen binding fragment binds to both monomeric TL1A and trimeric TL1A, and wherein the antibody or antigen binding fragment blocks interaction of TL1A to DR3. In some embodiments, the first therapeutically effective amount comprises 2000, 1950, 1900, 1850, 1800, 1750, 1700, 1650, 1600, 1550, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, or 200 mg.

In some embodiments, the anti-TL1A antibody or antigen binding fragment comprises a heavy chain variable region comprising: an HCDR1 comprising an amino acid sequence set forth by SEQ ID NO: 1, an HCDR2 comprising an amino acid sequence set forth by any one of SEQ ID NOS: 2-5, and an HCDR3 comprising an amino acid sequence set forth by any one of SEQ ID NOS: 6-9; and a light chain variable region comprising an LCDR1 comprising an amino acid sequence set forth by SEQ ID NO: 10, an LCDR2 comprising an amino acid sequence set forth by SEQ ID NO: 11, an LCDR3 comprising an amino acid sequence set forth by any one of SEQ ID NOS: 12-15.

In some embodiments, the anti-TL1A antibody or antigen binding fragment comprises a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise no or fewer than nine amino acid modification(s) from the human IGHV1-46*02 framework and the human IGKV3-20 framework.

In some embodiments, the anti-TL1A antibody or antigen binding fragment comprises a heavy chain variable domain comprising an amino acid sequence at least 96% identical to any one of SEQ ID NOS: 101-169, and a light chain variable domain comprising an amino acid sequence at least 96% identical to any one of SEQ ID NOS: 201-220.

In some embodiments, the anti-TL1A antibody or antigen binding fragment comprises a heavy chain variable region comprising SEQ ID NO: 301 XIVQLVQSGAEVKKPGASVKVSCKAS[HCDR1]WVX2QX3PGQGLEWX4G[HCDR2]RX5TX6TX7DTSTSTX8YX9ELSSLRSEDTAVYYCAR[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRFSGSGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V, wherein HCDR1 comprises an amino acid sequence set forth by SEQ ID NO: 1, HCDR2 comprises an amino acid sequence set forth by any one of SEQ ID NOS: 2-5, HCDR3 comprises an amino acid sequence set forth by any one of SEQ ID NOS: 6-9, LCDR1 comprises an amino acid sequence set forth by SEQ ID NO: 10, LCDR2 comprises an amino acid sequence set forth by SEQ ID NO: 11, and LCDR3 comprises an amino acid sequence set forth by any one of SEQ ID NOS: 12 or 13.

In some embodiments, the IL23 inhibitor specifically inhibits IL23. In some embodiments, the IL23 inhibitor inhibits IL23 and does not bind to IL12.

In some embodiments, the IL23 inhibitor comprises ustekinumab. In some embodiments, the second therapeutically effective amount comprises (i) 45 mg/dose if the subject has a body weight of less than or equal to 100 kg or (ii) 90 mg/dose if the subject has a body weight of greater than 100 kg.

In some embodiments, the IL23 inhibitor comprises guselkumab. In some embodiments, the second therapeutically effective amount comprises a dose of 100 mg administered in an initial dose, 4 weeks after the initial dose and every 8 weeks after the dose at 4 weeks.

In some embodiments, the IL23 inhibitor comprises risankizumab. In some embodiments, the second therapeutically effective amount comprises a dose of 150 mg by subcutaneous injection at Week 0, Week 4, and every 12 weeks thereafter.

In some embodiments, the IL23 inhibitor comprises brazikumab. In some embodiments, the second therapeutically effective amount comprises (a) 720-1440 mg on or about days 1, 29, and 57 delivered intravenously, followed by (b) about 240 mg delivered subcutaneously on or about day 85 and about every 4 weeks thereafter through at least week 48.

In some embodiments, the IL23 inhibitor comprises mirikizumab. In some embodiments, the second therapeutically effective amount comprises at least one induction dose of about 200 mg to about 1200 mg of the mirikizumab and at least one maintenance dose of about 100 mg to about 600 mg of the mirikizumab.

In some embodiments, the IL23 inhibitor comprises tildrakizumab. In some embodiments, the second therapeutically effective amount comprises a dose of 100 mg of the tildrakizumab at Weeks 0, 4, and every twelve weeks thereafter up to 52 weeks.

In some embodiments, the IL23 inhibitor comprises briakinumab. In some embodiments, the second therapeutically effective amount comprises (i) a first dose amount of 180 mg to 220 mg of the antibody or antigen-binding domain thereof, at week 0, and for the same first dose amount of the antibody or antigen-binding domain thereof at week 4, and (ii) a second dose amount of 80 mg to 120 mg of the antibody or antigen-binding domain thereof every 4 weeks thereafter.

In some embodiments, the third therapeutically effective amount is identical to the first therapeutically effective amount or the third therapeutically effective amount is less than the first therapeutically effective amount.

In some embodiments, the fourth therapeutically effective amount is identical to the second therapeutically effective amount or the fourth therapeutically effective amount is less than the second therapeutically effective amount.

### 5. BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1C show chromatograms for analytical size exclusion chromatography of anti-TL1A antibodies. The large peaks (main peak) correspond to monomeric fraction. The percentage of monomeric sample is indicated for each antibody. FIG. 1A shows chromatographs for antibodies A193, A194, and A195. FIG. 1B shows chromatographs for antibodies A196, A197, and A198. FIG. 1C shows chromatographs for antibodies A199, A200, and A201.

FIG. 2 depicts inhibition of interferon gamma in human blood with an anti-TL1A antibody.

FIG. 3A depicts the comparison between the predicted and measured viscosity. FIGS. 3B-3D depict a PLS model demonstrating effect of pH and protein concentration on viscosity. FIG. 3B shows a PLS graph (x-axis is pH, y-axis is protein concentration (mg/ml), z-axis is viscosity (mPa-s) for the PLS graphs), FIG. 3C shows a model of the predicted viscosity (y-axis, mPa-s) versus anti-TL1A antibody concentration (x-axis) in mg/mL, and FIG. 3D shows a model of the estimated viscosity (y-axis, mPa-s) versus actual viscosity (x-axis, mPa-s). FIG. 3E depicts the effects of pH versus acetate concentration on viscosity. FIG. 3F shows the effect of sucrose versus NaCl on viscosity. FIG. 3G depicts the effect of Arg-HCl versus Lys-HCl on viscosity. Viscosity units are in mPa-s. The arrow points to the region of highest viscosity. The star corresponds to the region of lowest viscosity.

FIG. 4A depicts the PLS1 model for the effect on high molecular weight (HMW) aggregates. FIG. 4B depicts the effect of pH versus acetate on aggregation. FIG. 4C depicts the effect of sucrose versus NaCl concentration. FIG. 4D depicts the effect of Arg-HCl versus Lys-HCl on aggregation. FIG. 4E depicts the effect of sucrose concentration versus Lys-HCl concentration.

FIG. 5A depicts the predicted versus measured loss of main peak at 2 weeks and 25° C. FIG. 5B depicts the effect of pH and protein concentration on the loss of main peak in the CEX profile. FIG. 5C depicts the effect of pH and acetate concentration on the loss of main peak in the CEX profile. FIG. 5D depicts the effect of sucrose and NaCl concentration on the loss of main peak in the CEX profile. FIG. 5E depicts the effect of Lys-HCl and sucrose concentration on the loss of main peak in the CEX profile.

FIG. 6A depicts the loss of monomer by SEC with agitation. FIG. 6B depicts the loss of monomer by SEC with freeze-thaw.

FIG. 7A depicts the binding of an anti-TL1A antibody to cynomolgus and human TL1A, but not to mouse or rat TL1A. ELISA for each protein was performed at least three times. The data from a representative experiment are shown and are mean±SD. Abbreviations: A=absorbance, Ab=antibody, Cyno=cynomolgus, nm=nanometer, nM=nanomolar. FIG. 7B depicts mean levels of sTL1A increased with increasing IV doses of anti-TL1A to cynomolgus monkeys, as measured in an ELISA. Samples were assayed in triplicate, on two separate occasions. Data presented are the mean TL1A concentrations of three animals per group±SD. Samples collected from animals administered isotype control antibody are shown in circles, samples collected from animals administered anti-TL1A are shown in the triangles and square. Abbreviations: hr=hour, kg=kilogram, mg=milligram, mL=milliliter, ng=nanogram; TL1A=tumor necrosis factor-like cytokine 1A.

FIG. 8 demonstrates that TL1A drives inflammation and fibrosis through binding to DR3.

FIGS. 9A-9C demonstrates size-exclusion chromatography (SEC) profiles of recombinant human TL1A (rhTL1A). Briefly, rhTL1A was labeled with Alexa fluor 488 (AF488) and spiked into normal human serum (NHS). In FIG. 9A, when injected alone, rhTL1A SEC profile shows two peaks on SEC, representing trimeric and monomeric forms of TL1A. In FIG. 9B, when rhTL1A is pre-incubated with a control reference antibody, the trimeric peak was shifted leftward, indicating a larger complex formation of the reference antibody and trimeric rhTL1A. There was no shift in the monomeric peak, indicating that the reference antibody only binds to the trimeric rhTL1A. In FIG. 9C, when rhTL1A is pre-incubated with A219, both the trimeric and the monomeric rhTL1A peaks were shifted, thus indicating that A219 binds both trimeric and monomeric forms of TL1A.

FIG. 10A depicts a whole-body physiologically based pharmacokinetic (PBPK) model. FIG. 10B depicts a tissue-level diagram of the integrated whole-body PBPK model used to characterize the PK of the monoclonal antibody (mAb), ligand, and complex between mAb and ligand.

FIG. 11A depicts the comparison of the pharmacokinetics of the mAb as predicted by the integrated whole-body PBPK (solid curve) with the pharmacokinetics of the mAb as observed in normal healthy volunteers (various points with points from the same subject shown by the same format), in each case after injection of A219 at the indicated dose. FIG. 11B depicts the comparison of the TL1A concentration as predicted by the integrated whole-body PBPK with the TL1A concentration as observed in normal healthy volunteers, in each case after injection of A219 at the indicated dose.

FIG. 12A depicts the observed concentration of TL1A in serum after injecting (i) an anti-TL1A antibody A219 that binds to both TL1A monomer and trimer (shown in red, top of the 2 curves, and the observed data points accompanying such curve) and (ii) a control reference anti-TL1A antibody that binds to only TL1A trimer (shown in blue, bottom of the 2 curves, and the observed data points accompanying such curve). In FIG. 12A, solid curves depict the prediction from the model and various dots depict the observations from subjects injected with the indicated antibodies. FIG. 12B depicts the predicted total TL1A concentration (monomer and trimer, solid curve and the observed data points accompanying such curve), the monomer TL1A concentration (fine dotted line), and the trimer TL1A concentration (coarse dotted line), in each case at the basal level (no injection of any anti-TL1A antibodies). FIG. 12C depicts the serum TL1A concentration in normal healthy volunteers (NHV) and UC patients, as predicted by the whole-body PBPK model (solid lines, upper line for UC patient and lower line for NHV) and as observed (various points).

FIGS. 13A-13B demonstrate the fitness of the model. FIG. 13A depicts the observed concentration of TL1A in serum of NHVs after injecting an anti-TL1A antibody that binds to only TL1A trimer (dots) and the prediction of the model (solid curve) that fits the observations at the indicated dose. Q2WX3=every 2 weeks for three times. FIG. 13B depicts the observed concentration of TL1A in serum of UC patients after injecting an anti-TL1A antibody that binds to only TL1A trimer (dots) and the prediction of the model (solid curve) that fits the observations at the indicated dose. Q2WX7=every 2 weeks for seven times. FIG. 13C depicts the concentration of TL1A in intestine of NHV (black, solid, lower line of the two lines as predicted from the model and the observed data points accompanying such line) and the concentration of TL1A in the intestine of UC patient (red, solid, upper line of the two lines).

FIGS. 14A-14B depict the baseline concentration of TL1A based on various parameters of TL1A production in intestine (14A) and in serum (14B). In FIGS. 14A-14B, 1× would be the baseline in NHV; 25×, 50×, 75×, and 100× indicate various parameters of TL1A over-production in intestine.

FIGS. 15A-15V depict the concentration of free soluble TL1A in tissue as determined by the whole-body PBPK model according to various parameters of TL1A overproduction under various dose regimen of anti-TL1A antibody A219 as indicated. FIG. 15W depicts the free soluble TL1A in tissue as determined by the whole-body PBPK model according to various parameters of TL1A overproduction under the dose regimen of a reference anti-TL1A antibody as indicated. FIGS. 15X-15Z depict the comparison of the modeled free soluble TL1A concentration in subjects treated with a reference anti-TL1A antibody (red, the upper curve of the two curves) or A219 (green, the lower curve of the two curves). In FIG. 15W-15Z, reference antibody light chain sequence is SEQ ID NO: 382, heavy chain sequence is SEQ ID NO: 383, and the whole-body PBPK model uses a rapid equilibrium between the monomeric and trimeric form of TL1A with a continuous 60:40 ratio of monomer and trimer as observed. The black solid lines in FIGS. 15A-15Z indicate the TL1A concentration in the tissue of NHV. Q2W=every 2 weeks. Q4W=every 4 weeks. SC=subcutaneous. LD=loading dose (the first dose). 4W=week 4. D1=day 1. W 2, 6, 10=week 2, week 6, and week 10. W 2, 4, 6, 10=week 2, week 4, week 6, and week 10. EOW=every other week. W 4, 8, 12=week 4, week 8, and week 12. W 2, 4, 8, 12=week 2, week 4, week 8, and week 12. sTL1A=soluble TL1A.

FIGS. 16A-16H depict the goodness of fit plots for A219 with the population PK model.

FIG. 17A depicts the visual predictive check for the A219 concentration predicted from the popPK model against the observed A219 concentration. FIG. 17B depicts an induction dose selected in the popPK model to rapidly achieve steady state concentration.

FIG. 18A depicts the study schema for induction period for the phase 2 clinical trial for A219 in UC. FIG. 18B depicts the study schema for open-label extension period for the phase 2 clinical trial for A219 in UC.

FIG. 19 depicts the study schema for the phase 2 clinical trial for A219 in CD.

FIG. 20 depicts osmotic pressures at 5° C. measured for the stability of A219 samples of various formulations at TO, 3 and 6 months.

FIG. 21 depicts A219 protein concentration at 5° C. measured for evaluating the stability of A219 samples of various formulations at TO, 3 and 6 months.

FIG. 22 depicts pH at 5° C. measured for the evaluating the stability A219 samples of various formulations at TO, 3 and 6 months.

FIG. 23A depicts viscosity data for TO and 3M for Formulations 1 to 5 at 25° C.;

FIG. 23B depicts viscosity data for TO and 3M for Formulations 6 to 8 at 25° C.

FIG. 24A depicts monomer contents for formulations at 5° C. as measured by SEC;

FIG. 24B depicts loss of monomer (main peak) per month for the formulations at 5° C. as determined by SEC; FIG. 24C depicts monomer contents for formulations at 25° C. as measured by SEC; FIG. 24D depicts loss of monomer (main peak) per month for the formulations at 5° C. as determined by SEC.

FIG. 25A depicts the relative area (%) of the main peak for formulations at 5° C. as characterized by cation exchange chromatography; FIG. 25B depicts the loss of main peak (Rel. Area (%) per month) for the formulations at 5° C. as determined by cation exchange chromatography; FIG. 25C depicts the relative area (%) of the main peak for formulations at 25° C. as characterized by cation exchange chromatography; FIG. 25D depicts the loss of main peak (Rel. Area (%) per month) for the formulations at 25° C. as determined by cation exchange chromatography.

FIG. 26A depicts predicted vs. measured values according to the PLS model using monomer loss by SEC for samples stored for 2 months at 25° C. as the endpoint; FIG. 26B depicts effect of pH and protein according to the PLS model using monomer loss by SEC for samples stored for 2 months at 25° C. as the endpoint. In FIG. 26B, the sucrose concentration was fixed at 200 mM. FIG. 26C depicts effect of pH and acetate according to the PLS model using monomer loss by SEC for samples stored for 2 months at 25° C. as the endpoint. In FIG. 26C, the sucrose concentration was fixed at 200 mM. FIG. 26D depicts effect of sucrose and lysine according to the PLS model using monomer loss by SEC for samples stored for 2 months at 25° C. as the endpoint. In FIG. 26D, the protein concentration was fixed at 150 mg/mL, pH at 5.5 and acetate at 20 mM. FIG. 26E depicts effect of glycine and NaCl according to the PLS model using monomer loss by SEC for samples stored for 2 months at 25° C. as the endpoint. In FIG. 26E, the protein concentration was fixed at 150 mg/mL, pH at 5.5 and acetate at 20 mM.

In FIGS. 20, 21, 22, 23A-23B, 24A-24D, 25A-25D, and 26A-26E, the formulations 1-8 (F01-F08, Form. 1-8, or simply 1-8) referenced therein are the formulations 1-8 as described in Table 31 of Example 24.

FIG. 27A shows geometric mean serum A219 concentration-time profiles following single doses of A219 administered as IV infusion (Linear Scale) (SAD study). FIG. 27B shows geometric mean serum A219 concentration-time profiles following multiple doses of A219 Q2W administered as IV infusion-day 29 (linear scale) (MAD study). Q2W=every 2 weeks.

FIG. 28A shows geometric mean serum sTL1A concentration versus nominal time following single dose of A219 administered as IV Infusion (semi-log scale) (SAD study). FIG. 28B geometric mean serum sTL1A concentration versus nominal time following multiple doses of A219 Q2W administered as IV infusion (semi-log scale) (MAD study).

FIG. 29A shows total A219 concentration in the central compartment (in circulation) in SAD as predicted by the model (curves) and as determined in the phase I trial (dots). FIG. 29B shows total soluble TL1A in the central compartment (circulation) in SAD as predicted by the model (curves) and as determined in the phase I trial. FIG. 29C shows total A219 concentration in the central compartment (in circulation) in MAD as predicted by the model (curves) and as determined in the phase I trial (dots). FIG. 29D shows total soluble TL1A in the central compartment (circulation) in MAD as predicted by the model (curves) and as determined in the phase I trial (dots). The predicted curves fitted with the measured data points. FIGS. 29E-29K show model prediction for and the data of a control reference antibody that binds only to TL1A trimer (light chain SEQ ID NO: 382 and heavy chain SEQ ID NO: 383) with regard to (1) phase I single ascending dose data (FIGS. 29E and 29F), (2) phase I multiple ascending dose data (FIGS. 29G and 29H), and (3) phase II data on PK & total sTL1A levels (FIGS. 291 and 29J). The IBD specific parameters were then calibrated to capture free tissue TL1A levels in the gut (FIG. 29K) as observed with the control reference antibody (light chain SEQ ID NO: 382 and heavy chain SEQ ID NO: 383). NR=non-responder and R=responder.

FIG. 30A shows doses of A219 determined from the validated model that can bring the free TL1A concentration in the patient's diseased tissue to below the TL1A concentration of a healthy subject. FIG. 30B shows the percent reduction of the free TL1A in the diseased tissue after administering doses of A219 as determined from the model. IV_4×=1000 mg loading dose, 3×500 mg on days 14, 42, 70. SC dosing 240 mg Q1W or Q2W. FIG. 30C shows that, in a head-to-head comparison in the validated model, anti-TL1A antibodies that bind to both TL1A monomer and trimer engaged more (3.5 fold more) TL1A in circulation than anti-TL1A antibodies that only bind to TL1A trimer. FIG. 30D shows that, in a head-to-head comparison in the validated model, anti-TL1A antibodies that bind to both TL1A monomer and trimer also resulted in higher percentage of TL1A reduction of TL1A in diseased tissue (about 100%) when compared to anti-TL1A antibodies that only bind to TL1A trimer.

FIG. 31A shows the diagram of a popPK model. FIG. 31B shows the comparison of the A219 concentration predicted from the popPK model and the A219 concentration observed in the population of subjects in phase I clinical trial via a linear regression plot. FIG. 31C shows the comparison of the TL1A concentration predicted from the popPK model and the TL1A concentration observed in the population of subjects in phase I clinical trial via a linear regression plot. FIG. 31D shows the comparison of the A219 concentration predicted from the popPK model and the A219 concentration observed in the population of subjects in phase I clinical trial via a time series plot. FIG. 31E shows the comparison of the TL1A concentration predicted from the popPK model and the TL1A concentration observed in the population of subjects in phase I clinical trial via a time series plot.

FIGS. 32A-32H show the A219 and TL1A engagement (TL1A concentration in serum) predicted from the validated popPK model under various A219 doses. FIGS. 32A and 32B show A219 concentration (32A) and TL1A concentration (32B) in circulation with a dosing regimen of induction with 500 mg Q2W (6 doses) up to week 10 and extension with 500 mg Q2W from week 12 to week 52 (20 doses). FIGS. 32C and 32D show A219 concentration (32C) and TL1A concentration (32D) in circulation with a dosing regimen of induction with 500 mg Q2W (6 doses) up to week 10 and extension with 500 mg Q4W from week 12 to week 52 (10 doses). FIGS. 32E and 32F show A219 concentration (32E) and TL1A concentration (32F) in circulation with a dosing regimen of induction with 500 mg Q2W (6 doses) up to week 10 and extension with 100 mg Q2W from week 12 to week 52 (20 doses). FIGS. 32G and 32H show A219 concentration (32G) and TL1A concentration (32H) with a dosing regimen of induction with 500 mg Q2W (6 doses) up to week 10 and extension with 250 mg Q4W from week 12 to week 52 (10 doses).

FIGS. 33A-33B shows gene expression analysis of TL1A/DR3 and IL23/IL23R pathway components in immune cells from IBD tissue biopsies. FIG. 33A depicts single cell RNAseq data clustered based on gene expression at the single cell level and identifies major immune cells clusters and subsets. In FIG. 33B, cells expressing IL23A (IL23), IL12A (IL12), IL23R (IL23R, IL12RB1), IL12R (IL12RB2, IL12RB1), TL1A (TNFSF15) and DR3 (TNFRSF25) were shown and were colocalized to immune cell subsets expressing them with correspondence to FIG. 33A. IEL=intraepithelial lymphocyte, LP=lamina propria, DC=dendritic cell, GC=germinal center B cell, MT=mitochondria, Tregs=regulatory T cells, ILCs=innate lymphoid cells.

FIG. 34A shows single cell RNAseq data clustered based on gene expression at the single cell level and identifies major stromal cell clusters and subsets. In FIG. 34B, cells expressing TL1A (TNFSF15), DR3 (TNFRSF25), IL23A, IL12A, IL23R, IL12RB1 and IL12RB2 were shown and were colocalized to stromal cell subsets expressing them with correspondence to FIG. 34A.

FIG. 35A shows IL23R and DR3 expression in single cell RNAseq data in biopsy samples from UC patients. In FIG. 35A, CD4+ and CD8+ inflammatory T cells as well as ILCs expressed DR3 and IL23R. FIG. 35B shows that inflamed tissue from IBD subjects had increased co-expression of IL23R and DR3 in T cells.

FIG. 36 shows studies comparing anti-TL1A treatment alone, anti-IL23 treatment alone, and anti-TL1A+anti-IL23 combination therapy in T cell transfer mouse colitis models.

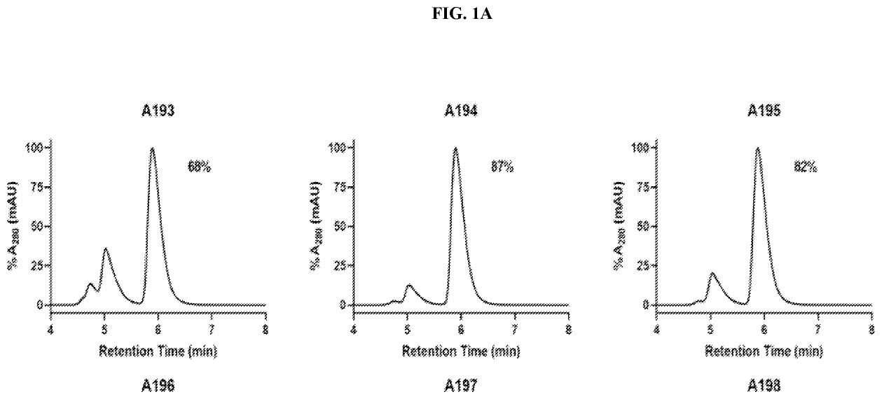

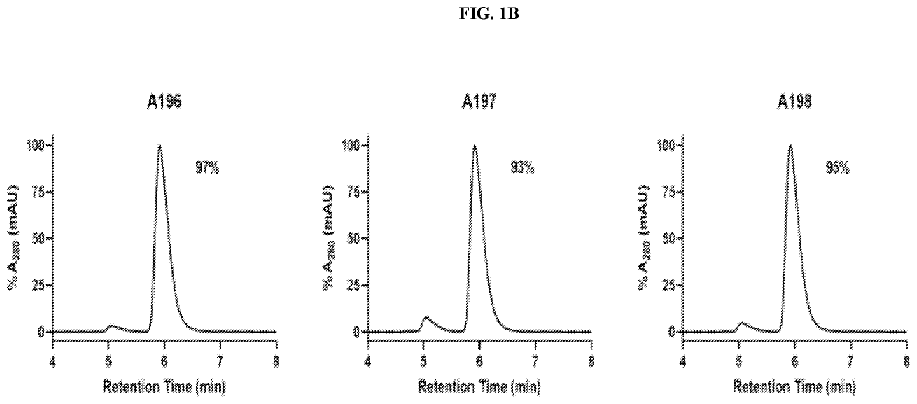

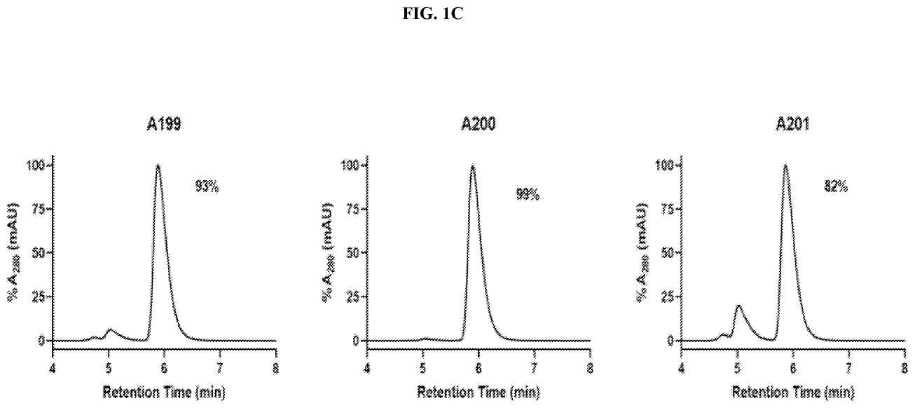

### 6. DESCRIPTION OF THE INVENTION

IL-23 is a heterodimeric cytokine composed of a unique p19 subunit and a common p40 subunit shared with IL-12 [10]. IL-23 engages with the heterodimeric IL-23 receptor (consisting of an IL-23R chain and an IL-12RB1 chain), activates intracellular JAKs (mainly through TYK2 and JAK2) and signal transducer and activator of transcription (STAT) pathways, among other signaling factors, which in turn regulates transcription of downstream genes. IL23 is one of the key promotors of the T helper 17 (Th17) cell pathway which has been implicated in many inflammatory diseases and conditions. The disclosure provides that blockade of IL23 alongside the blockade of TL1A can provide significant benefits in the treatment of inflammatory diseases and conditions. Multiple inhibitors of IL23 have been developed, tested and/or currently used in clinical to treat inflammatory diseases and conditions, including ustekinumab, guselkumab, risankizumab, brazikumab, mirikizumab, tildrakizumab, and briakinumab, all of which are provided herein as embodiment of IL23 inhibitors for the combination therapy provided herein.

TL1A is a cytokine that is secreted by antigen-presenting cells, T cells, and endothelial cells. TL1A signals through death receptor 3 (DR3), a TNF-family receptor that is found primarily on T cells, natural killer (NK) and NK-T cells, innate lymphoid cells (ILC), fibroblasts, and epithelial cells and potently drives Th1, Th2, Th9 and Th17 responses. In addition, it is induced in antigen-presenting cells by toll like receptor (TLR) ligands and FcR cross-linking and in T cells by T cell receptor (TCR) stimulation.

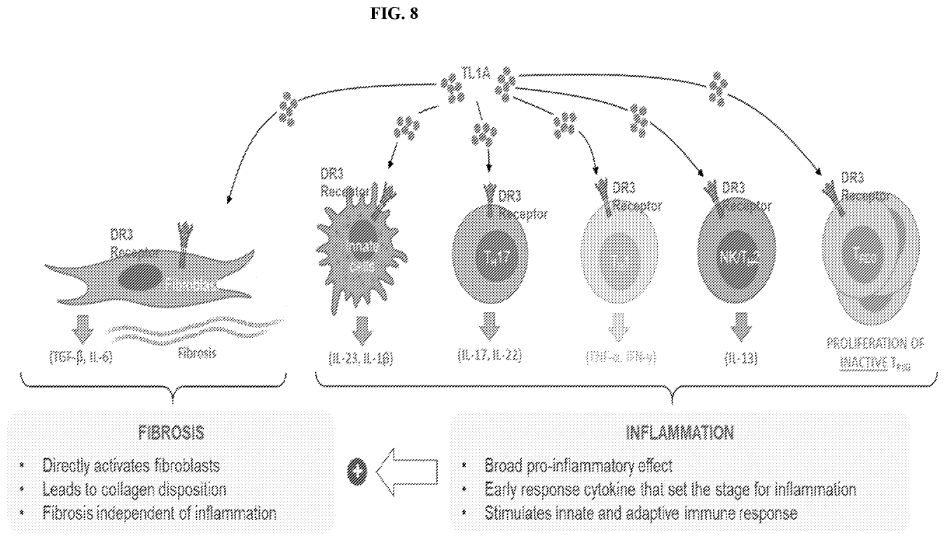

FIG. 8 demonstrates how TL1A binding to DR3 independently drives inflammation and fibrosis. TL1A binding to DR3 on innate and T cells leads to an early cytokine response (release of IL-23, IL-1B, IL-17, IL-22, TNF-α, IFN-γ, IL-13) that sets the stage for inflammation, and stimulates innate and adaptive immune response. For instance, through binding to DR3, TL1A potentially drives inflammatory Th1 and Th17 responses. Further, binding of TL1A to DR3 on fibroblasts directly activates fibroblasts, and leads to collagen disposition and fibrosis independent of inflammation. While levels of circulating TL1A are low in healthy subjects, they are elevated in patients suffering from many auto-immune diseases, and TL1A has been shown to be upregulated in mucosa and serum of patients with IBD. In mice, chronic TL1A expression causes structuring disease caused by increased collagen deposition. In dextran sodium sulfate (DSS) and adoptive transfer mouse models, when challenged with DSS, TL1A transgenic mice develop more severe colitis than wild-type animals, and antibodies against TL1A led to reduced inflammation, lowered collagen levels, and reversal of fibrosis, even when treatment was administered late in the course of disease, after inflammation and fibrosis has been established. Furthermore, TL1A polymorphisms have been shown to be associated with susceptibility to IBD and with disease severity.

Fibrosis is a significant clinical phenotype exhibited by IBD patients. Seventy percent of Crohn's disease (CD) patients develop stricture/perforation, and stricture is the leading indication for surgery in CD. Unfortunately, anti-inflammatory agent use over the past decade has not materially changed the rate of structuring disease or need for surgery. Further, in ulcerative colitis (UC), subclinical fibrosis has significant implications on patient symptoms. For instance, subclinical fibrosis could contribute to symptoms of diarrhea, abdominal pain, urgency, and incontinence. Subclinical fibrosis is also the potential explanation for persistent symptoms after resolution of inflammation. In addition, a Cleveland Clinic study of 89 consecutive colectomy specimens revealed submucosal fibrosis in 100% of the specimens. Thus, treatment of fibrosis constitutes an unmet need in IBD.

The potential for TL1A as a therapeutic target in intestinal fibrosis has been demonstrated in a study evaluating the effect of anti-TL1A antibodies in mouse models of IBD. In these studies, two mouse models of chronic colitis were utilized: adoptive T cell transfer and chronic DSS. In both models, a neutralizing TL1A monoclonal antibody (mAb) or an isotype control antibody was administered two times per week in mice (T cell transfer n=14; DSS n=28) with an established colitis. In both disease models, treatment with the TL1A mAb reduced colonic collagen deposition levels back to those seen in healthy control mice, suggesting that blocking TL1A signaling not only prevented progression of colonic fibrosis, but also reversed established fibrosis to similar levels measured prior to the onset of inflammation. This data indicates that intestinal fibrosis mediated by increased levels of TL1A may be treated with an anti-TL1A antibody.

In one aspect, provided herein are humanized monoclonal antibodies that bind to both membrane-bound and soluble forms of TL1A with high affinity and specificity and block the binding of TL1A to its functional receptor DR3. By targeting both inflammation and fibrosis, such antibodies have the potential to improve outcomes for IBD patients, such as those with increased TL1A expression.

Without being bound by the theory, the disclosure provides that in instances when a particular cell type expresses both TL1A/DR3 and IL23/IL23R pathway receptors, blockade of both is needed in order to effectively neutralize the pro-inflammatory function of that cell. Additionally, cells expressing either pathway alone can also contribute to inflammation and disease independently and blocking either pathway alone can leave a significant population of inflammation-causing immune cells unchecked. Thus, the disclosure provides that combining therapeutics to block both TL1A/DR3 and IL23/IL23R pathways can be more efficacious than blocking either pathway alone. Combining therapeutics to block both TL1A/DR3 and IL23/IL23R pathways can result in blockade of multiple inflammation-causing cell types and result in a synergistic reduction in inflammation.

In some embodiments for the various methods, compositions, combination therapies, the TL1A inhibitors, and/or the IL23 inhibitors provided herein, including in Sections 2, 3, 4, and 5, the inflammatory disease or condition comprises or consists of inflammatory bowel disease (IBD). In some embodiments for the various methods, compositions, combination therapies, the TL1A inhibitors, and/or the IL23 inhibitors provided herein, including in Sections 2, 3, 4, and 5, the inflammatory disease or condition comprises or consists of ulcerative colitis (UC). In some embodiments for the various methods, compositions, combination therapies, the TL1A inhibitors, and/or the IL23 inhibitors provided herein, including in Sections 2, 3, 4, and 5, the inflammatory disease or condition comprises or consists of indeterminate colitis. In some embodiments for the various methods, compositions, combination therapies, the TL1A inhibitors, and/or the IL23 inhibitors provided herein, including in Sections 2, 3, 4, and 5, the inflammatory disease or condition comprises or consists of moderately to severely active UC. In some embodiments for the various methods, compositions, combination therapies, the TL1A inhibitors, and/or the IL23 inhibitors provided herein, including in Sections 2, 3, 4, and 5, the inflammatory disease or condition comprises or consists of Crohn's Disease (CD).

### 6.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Green and Sambrook, Molecular Cloning: A Laboratory Manual (4th ed. 2012); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

### 6.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein, the term “inhibitor,” when used in reference to a target molecule such as TL1A or IL23, is intended to mean a molecule that is capable of inhibiting, decreasing, attenuating, reducing, or otherwise completely abolishing the protein level or one or more of the biological activities or functions of the target molecule (such as TL1A or IL23). As such, inhibitors of a target include inhibitors of the target activity or function, inhibitors of the target expression, or inhibitors of the target protein level. For example, an inhibitor of a TL1A activity or function includes a molecule that can block, inhibit, attenuate, or reduce TL1A-mediated or TL1A-dependent signaling in a cell expressing a TL1A. An inhibitor of a TL1A expression also includes a molecule that can block, inhibit, attenuate, or reduce TL1A expression or TL1A protein levels in the cells. In some examples, an inhibitor of TL1A further includes molecules that can block, inhibit, attenuate, or reduce TL1A binding to a natural TL1A receptor such as Death Receptor 3 (DR3). An “inhibitor” of TL1A is “inhibitory” to TL1A or TL1A function. In some embodiments, provided herein are inhibitory anti-TL1A antibodies or antigen binding fragments thereof. In some embodiments, TL1A inhibitors provided herein are siRNA molecules against TL1A mRNA.

Similarly, an inhibitor of an IL23 activity or function includes a molecule that can block, inhibit, attenuate, or reduce IL23-mediated or IL23-dependent signaling in a cell responding to IL23. An inhibitor of IL23 expression also includes a molecule that can block, inhibit, attenuate, or reduce IL23 expression from IL23-expressing cells or IL23 protein levels in the subject (such as in the diseased tissue of the subject, in the blood of the subject, or other bodily fluid of the subject). In some examples, an inhibitor of IL23 further includes molecules that can block, inhibit, attenuate, or reduce IL23 binding to a natural IL23 receptor, such as IL23R or the complex of an IL-23R chain and an IL-12RB1 chain. An “inhibitor” of IL23 is “inhibitory” to IL23 or IL23 function. In some embodiments, provided herein are small molecule IL23 inhibitors. In some embodiments, provided herein are inhibitory anti-IL23 antibodies or antigen binding fragments thereof. In some embodiments, IL23 inhibitors provided herein are siRNA molecules against IL23 mRNA.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as TL1A, is the affinity of the antibody or functional fragment for that epitope. The ratio of dissociation rate (k_{off}) to association rate (kₒₙ) of an antibody to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent TL1A trimer, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). As described above, the affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” can be measured by assays known in the art, for example by a binding assay. The K_{D} can be measured in a RIA, for example, performed with the Fab version of an antibody of interest and its antigen (Chen et al., 1999, J. Mol Biol 293:865-81). The K_{D} or K_{D} value can also be measured by using surface plasmon resonance assays by Biacore™, using, for example, a Biacore® TM-2000 or a Biacore® TM-3000, or by biolayer interferometry using, for example, the Octet® QK384 system. An “on-rate” or “rate of association” or “association rate” or “kₒₙ” can also be determined with the same surface plasmon resonance or biolayer interferometry techniques described above using, for example, a Biacorex®-2000 or a Biacorex®-3000, or the Octet® QK384 system.

The term “binding protein” refers to a protein comprising a portion (e.g., one or more binding regions such as CDRs) that binds to a target such as TL1A or IL23, and, optionally, a scaffold or framework portion (e.g., one or more scaffold or framework regions) that allows the binding portion to adopt a conformation that promotes binding of the binding protein to the polypeptide, fragment, or epitope of TL1A or IL23. Examples of such binding proteins include antibodies, such as a human antibody, a humanized antibody, a chimeric antibody, a recombinant antibody, a single chain antibody, a diabody, a triabody, a tetrabody, a Fab fragment, a F(ab′)₂ fragment, an IgD antibody, an IgE antibody, an IgM antibody, an IgG1 antibody, an IgG2 antibody, an IgG3 antibody, or an IgG4 antibody, and fragments thereof. The binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the binding protein as well as wholly synthetic scaffolds comprising, for example, a biocompatible polymer. See, e.g., Korndorfer et al., 2003, Proteins: Structure, Function, and Bioinformatics 53 (1): 121-29; and Roque et al., 2004, Biotechnol. Prog. 20:639-54. In addition, peptide antibody mimetics (“PAMs”) can be used, as well as scaffolds based on antibody mimetics utilizing fibronectin components as a scaffold. In the context of the present disclosure, a binding protein is said to specifically bind or selectively bind to the target, such as TL1A or IL23, for example, when the dissociation constant (K_{D}) is ≤10⁻⁷ M. In some embodiments, the binding proteins (e.g., antibodies) may specifically bind to the target, such as TL1A or IL23, with a K_{D} of from about 10⁻⁷ M to about 10⁻¹² M. In certain embodiments, the binding protein (e.g., antibody) may specifically bind to the target, such as TL1A or IL23, with high affinity when the K_{D} is ≤10⁻⁸ M or K_{D} is ≤10⁻⁹ M. In one embodiment, the binding proteins (e.g., antibodies) may specifically bind to the target, such as TL1A or IL23, with a K_{D} of from 1×10⁻⁹ M to 10×10⁻⁹ M as measured by Biacore®. In another embodiment, the binding proteins (e.g., antibodies) may specifically bind to the target, such as TL1A or IL23, with a K_{D} of from 0.1×10⁻⁹ M to 1×10⁻⁹ M as measured by KinExA™ (Sapidyne, Boise, ID). In yet another embodiment, the binding proteins (e.g., antibodies) specifically bind to the target, such as TL1A or IL23, expressed on cells with a K_{D} of from 0.1×10⁻⁹ M to 10×10⁻⁹ M. In certain embodiments, the binding proteins (e.g., antibodies) specifically bind to the target, such as TL1A or IL23, expressed on cells with a K_{D} of from 0.1×10⁻⁹ M to 1×10⁻⁹ M. In some embodiments, the binding proteins (e.g., antibodies) specifically bind to the target, such as TL1A or IL23, expressed on cells with a K_{D} of 1×10⁻⁹ M to 10×10⁻⁹ M. In certain embodiments, the binding proteins (e.g., antibodies) specifically bind to the target, such as TL1A or IL23, expressed on cells with a K_{D} of about 0.1×10⁻⁹ M, about 0.5×10⁻⁹ M, about 1×10⁻⁹ M, about 5×10⁻⁹ M, about 10×10⁻⁹ M, or any range or interval thereof.

The term “antibody,” “immunoglobulin,” or “Ig” is used interchangeably herein, and is used in the broadest sense and specifically covers antibodies such as individual anti-TL1A or anti-IL23 monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including for example a TL1A polypeptide, a TL1A fragment, or a TL1A epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments such as TL1A-binding fragments or IL23-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments such as TL1A-binding fragments or IL23-binding fragments fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen such as TL1A or IL23 (e.g., one or more CDRs of an anti-TL1A antibody or one or more CDRs of an anti-IL23 antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Anti-TL1A antibodies can be inhibitory antibodies. Provided herein are inhibitory antibodies to TL1A, including antibodies that reduce or block TL1A signaling, reduce or abolish TL1A protein level, and/or block or reduce binding between TL1A and DR3.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994).

An “antigen” is a predetermined antigen to which an antibody can selectively bind. A target antigen may be a polypeptide, carbohydrate, nucleic acid, lipid, hapten, or other naturally occurring or synthetic compound. In some embodiments, the target antigen is a polypeptide.

The terms “antigen-binding fragment,” “antigen-binding domain,” “antigen-binding region,” “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs).

The term “anti-TL1A” is an abbreviation for antibody or antigen binding fragment that binds TL1A. The term “anti-IL23” is an abbreviation for antibody or antigen binding fragment that binds IL23. The term “TL1A” is used interchangeably with “TL1a.”

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Accordingly, CDRs are variable region sequences interspersed within the framework region sequences. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme).

CDR regions are well known to those skilled in the art and have been defined by, for example, Kabat as the regions of most hypervariability within the antibody variable (V) domains (Kabat et al., 1997, J. Biol. Chem. 252:6609-16; Kabat, 1978, Adv. Prot. Chem. 32:1-75). CDR region sequences also have been defined structurally by Chothia as those residues that are not part of the conserved β-sheet framework, and thus are able to adapt different conformations (Chothia and Lesk, 1987, J. Mol. Biol. 196:901-17). Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art.

A number of CDR delineations are in use and are encompassed herein. The Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. The residues from each of these hypervariable regions or CDRs are noted below.

Additionally, a universal numbering system has been developed and widely adopted, ImMunoGeneTics (IMGT) Information System (Lafranc et al., 2003, Dev. Comp. Immunol. 27 (1): 55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309:657-70. Correspondence between the numbering system, including, for example, the Kabat numbering and the IMGT unique numbering system, is well known to one skilled in the art (see, e.g., Kabat, supra, Chothia and Lesk, supra; Martin, supra; Lefranc et al., supra).

| | IMGT | Kabat | AbM | Chothia | Contact |
|---|---|---|---|---|---|
| V_{H} CDR1 | 27-38 | 31-35 | 26-35 | 26-32 | 30-35 |
| V_{H} CDR2 | 56-65 | 50-65 | 50-58 | 53-55 | 47-58 |
| V_{H} CDR3 | 105-117 | 95-102 | 95-102 | 96-101 | 93-101 |
| V_{L} CDR1 | 27-38 | 24-34 | 24-34 | 26-32 | 30-36 |
| V_{L} CDR2 | 56-65 | 50-56 | 50-56 | 50-52 | 46-55 |
| V_{L} CDR3 | 105-117 | 89-97 | 89-97 | 91-96 | 89-96 |

Accordingly, the term “variable region residue numbering as in Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH. As used herein, the terms “HVR” and “CDR” are used interchangeably.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

In certain embodiments, the CDRs of the antibodies described herein can be defined by a method selected from Kabat, Chothia, IMGT, Aho, AbM, or combinations thereof.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while u and & contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. Light chain amino acid sequences are well known in the art. A light chain can be a human light chain.

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

Accordingly, in a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region. The humanized heavy chain variable domain may comprise IGHV1-46*02 framework with no or fewer than about 10, 9, 8, 7, 6, 5, 4, 3, 2, or 1 amino acid mutations. The humanized light chain variable domain may comprise IGKV3-20 framework with no or fewer than about 10, 9, 8, 7, 6, 5, 4, 3, 2, or 1 amino acid mutations.

A “human antibody” is one that possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1:755-68). Also available for the preparation of human monoclonal antibodies are methods described in Cole et al., Monoclonal Antibodies and Cancer Therapy 77 (1985); Boerner et al., 1991, J. Immunol. 147 (1): 86-95; and van Dijk and van de Winkel, 2001, Curr. Opin. Pharmacol. 5:368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6 (5): 561-66; Brüggemann and Taussing, 1997, Curr. Opin. Biotechnol. 8 (4): 455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

The terms “specifically bind to,” “specific binding,” and analogous terms when used in the context of one molecule binding to the other, means that one molecule binds to the other molecule with significantly higher affinity than to any cross-reactive antigen or off-target antigen (together as non-target antigen) as determined using experimental techniques, such as Surface Plasmon Resonance (SPR), fluorescence activated cell sorting (FACS) analysis, Kinetic Exclusion Assay (KinExA), isothermal titration calorimetry (ITC), radioimmunoassays (RIA) and enzyme linked immunosorbent assays (ELISAs). Typically a specific or selective reaction will be at least twice non-target signal or noise of non-target binding and may be more than 10 times non-target binding. See, e.g., Fundamental Immunology 332-36 (Paul ed., 2d ed. 1989) for a discussion regarding antibody specificity. An inhibitor which binds a target of interest (e.g., a target TL1A or IL23) is one that binds the target with sufficient affinity such that the inhibitor is useful as a therapeutic agent in targeting a cell or tissue expressing the target, and does not significantly cross-react with other proteins. In such embodiments, the extent of binding of the inhibitor to a “non-target” protein will be less than about 10% of the binding of the inhibitor to its particular target protein, for example, as determined by FACS analysis, SPR, KinExA, ITC, ELISA, or RIA. As is clear from the description herein, an inhibitor can be an antibody or antigen binding fragment thereof. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. As such, the term “specific binding,” “specifically binds to,” “specifically inhibits,” or “is specific for” a particular target as used herein refers to binding or inhibition where a molecule binds to or inhibits a particular target without substantially binding to or inhibiting a non-target. In certain embodiments, the TL1A inhibitor provided herein specifically binds to TL1A. In some embodiment, the anti-TL1A antibody provided herein specifically binds to TL1A. In certain embodiments, the IL23 inhibitor provided herein is specific for IL23 and does not bind to IL12. In some embodiments, the IL23 inhibitor provided herein binds both IL23 and IL12. In some embodiments, an antibody that specifically binds to a protein indicates that the antibody reacts or associates more frequently, more rapidly, with greater duration, with greater affinity, or with some combination of the above to the protein than with alternative substances, including unrelated proteins.

The term “effective amount” as used herein refers to the amount of an antibody, an inhibitor or pharmaceutical composition provided herein which is sufficient to result in the desired outcome. The term “therapeutically effective amount” as used herein refers to the amount of an antibody, an inhibitor or pharmaceutical composition provided herein which is sufficient to result in the desired outcome in a therapeutic treatment.

The term “chimeric antibody(ies)” refer to antibodies wherein the sequence of the immunoglobulin molecule is derived from two or more species. As a non-limiting example, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

In some embodiments, a protein such as an antibody described herein comprises a hydrophobic amino acid. Non-limiting exemplary hydrophobic amino acids include glycine (Gly), proline (Pro), phenylalanine (Phe), alanine (Ala), isoleucine (Ile), leucine (Leu), and valine (Val). In some embodiments, a protein such as an antibody described herein comprises a hydrophilic amino acid. Non-limiting exemplary hydrophilic amino acids include serine (Ser), threonine (Thr), aspartic acid (Asp), glutamic acid (Glu), cysteine (Cys), asparagine (Asn), glutamine (Gln), arginine (Arg), and histidine (His). In some embodiments, a protein such as an antibody described herein comprises an amphipathic amino acid. Non-limiting exemplary amphipathic amino acids include lysine (Lys), tryptophan (Trp), tyrosine (Tyr), and methionine (Met). In some embodiments, a protein such as an antibody described herein comprises an aliphatic amino acid. Non-limiting exemplary aliphatic amino acids include alanine (Ala), isoleucine (Ile), leucine (Leu) and valine (Val). In some embodiments, a protein such as an antibody described herein comprises an aromatic amino acid. Non-limiting exemplary aromatic amino acids include phenylalanine (Phe), tryptophan (Trp), and tyrosine (Tyr). In some embodiments, a protein such as an antibody described herein comprises an acidic amino acid. Non-limiting exemplary acidic amino acids include aspartic acid (Asp) and glutamic acid (Glu). In some embodiments, a protein such as an antibody described herein comprises a basic amino acid. Non-limiting exemplary basic amino acids include arginine (Arg), histidine (His), and lysine (Lys). In some embodiments, a protein such as an antibody described herein comprises a hydroxylic amino acid. Non-limiting exemplary hydroxylic amino acids include serine (Ser) and threonine (Thr). In some embodiments, a protein such as an antibody described herein comprises a sulfur-containing amino acid. Non-limiting exemplary sulfur-containing amino acids include cysteine (Cys) and methionine (Met). In some embodiments, a protein such as an antibody described herein comprises an amidic amino acid. Non-limiting exemplary amidic amino acids include asparagine (Asn) and glutamine (Gln).

The terms “polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the term “about” means mean within 10%, within 9%, within 8%, within 7%, within 6%, within 5%, within 4%, within 3%, within 2%, within 1%, or less of a given value, amount, or range. For instance, an antibody variable region comprising about 80% identity to a reference variable region may comprise 72% to 88% identity to the reference variable region.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain an anti-SIRPα antibody, for example, in isolated or purified form, together with a suitable amount of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

“Administer” “administering,” or “administration” refers to the act of injecting or otherwise physically delivering a substance as it exists outside the body (e.g., an anti-TL1A antibody or an IL23 inhibitor as described herein) into a patient, such as by mucosal, intradermal, intravenous, intramuscular delivery, and/or any other method of physical delivery described herein or known in the art.

As used herein, the term “combination therapy” is intended to mean the methods of treating an inflammatory disease or condition with the combination of an inhibitor of TL1A (“TL1A inhibitor”) and an inhibitor of interleukin 23 (“IL-23” or “IL23”) as provided herein, including in Sections 2, 4 (e.g. Section 4.7), and 5.

“Antibody-dependent cell-mediated cytotoxicity” or “ADCC” refers to a form of cytotoxicity in which secreted immunoglobulin bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. NK cells, the primary cells for mediating ADCC, express FcγRIII only, whereas monocytes express FcγRI, FcγRII, and FcγRIII. FR expression on hematopoietic cells is known (see, e.g., Ravetch and Kinet, 1991, Annu. Rev. Immunol. 9:457-92). To assess ADCC activity of a molecule of interest, an in vitro ADCC assay (see, e.g., U.S. Pat. Nos. 5,500,362 and 5,821,337) can be performed. Useful effector cells for such assays include peripheral blood mononuclear cells (PBMC) and Natural Killer (NK) cells. Alternatively or additionally, ADCC activity of the molecule of interest may be assessed in vivo, for example, in an animal model (see, e.g., Clynes et al., 1998, Proc. Natl. Acad. Sci. USA 95:652-56). Antibodies with little or no ADCC activity may be selected for use.

“Antibody-dependent cellular phagocytosis” or “ADCP” refers to the destruction of target cells via monocyte or macrophage-mediated phagocytosis when immunoglobulin bound onto Fc receptors (FcRs) present on certain phagocytotic cells (e.g., neutrophils, monocytes, and macrophages) enable these phagocytotic cells to bind specifically to an antigen-bearing target cell and subsequently kill the target cell. To assess ADCP activity of a molecule of interest, an in vitro ADCP assay (see, e.g., Bracher et al., 2007, J. Immunol. Methods 323:160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248:167-82). Antibodies with little or no ADCP activity may be selected for use.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay (see, e.g., Gazzano-Santoro et al., 1996, J. Immunol. Methods 202:163) may be performed. Polypeptide variants with altered Fc region amino acid sequences (polypeptides with a variant Fc region) and increased or decreased C1q binding capability have been described (see, e.g., U.S. Pat. No. 6,194,551; WO 1999/51642; Idusogie et al., 2000, J. Immunol. 164:4178-84). Antibodies with little or no CDC activity may be selected for use.

The term “variant” when used in relation to a protein (e.g. a therapeutic target or an antibody or antigen-binding fragment) can refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. By way of example, a variant of an anti-IL23 antibody may result from one or more (such as, for example, about 1 to about 25, about 1 to about 20, about 1 to about 15, about 1 to about 10, or about 1 to about 5) changes to an amino acid sequence of a native or previously unmodified anti-IL23 antibody. Variants may be naturally occurring, such as allelic or splice variants, or may be artificially constructed. Polypeptide variants may be prepared from the corresponding nucleic acid molecules encoding the variants. In some specific embodiments, the anti-IL23 antibody variant at least retains the functional activity of inhibiting IL23 activity. In some specific embodiments, the anti-TL1A antibody variant at least retains the functional activity of inhibiting TL1A expression or TL1A activity. In specific embodiments, an anti-TL1A antibody variant binds TL1A and/or is antagonistic to TL1A activity. In specific embodiments, an anti-IL23 antibody variant binds IL23 and/or is antagonistic to IL23 activity. In certain embodiments, the variant is encoded by a single nucleotide polymorphism (SNP) variant of a nucleic acid molecule that encodes anti-TL1A or anti-IL23 antibody VH or VL regions or subregions, such as one or more CDRs.

The disclosure provides that wherever embodiments are provided herein with the term “comprising,” the analogous embodiments described in terms of “consisting of” and/or “consisting essentially of” are also provided, if such analogous embodiments are not explicitly provided. The disclosure further provides that wherever embodiments are described herein with the phrase “consisting essentially of,” the analogous embodiments described in terms of “consisting of” are also provided. The disclosure also provides that wherever embodiments are described herein with the phrase “consisting of,” the analogous embodiments described in terms of “consisting essentially of” are also provided.

All applications, publications, patents and other references, GenBank citations and ATCC citations cited herein are incorporated by reference in their entirety. In case of conflict, the specification, including definitions, will control.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

The term “and/or” as used in a phrase with a list of members is intended to include all members individually and all combination of full or partial list of members. For example, a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 6.3 Therapeutic Agent Provided for the Combination to Treat Inflammatory Disease or Condition

Provided herein are inhibitors of TL1A (“TL1A inhibitors”) and inhibitors of IL23 (“IL23 inhibitors”) that can be used in a combination to treat inflammatory diseases or conditions, such as IBD, UC, or CD. The TL1A inhibitors that can be used in the combination therapy to treat inflammatory diseases or conditions, such as IBD, UC, or CD, are provided in Section 2, 4.3.1, and 5; the therapeutically effective amount (such as the dose and dosing regimen) for such TL1A inhibitors that can be used in the combination therapy are provided in Sections 2, 4.5, 4.6, 4.7 and 5; the IL23 inhibitors that can be used in the combination therapy to treat inflammatory diseases or conditions, such as IBD, UC, or CD, are provided in Sections 2, 4.3.2 and 5; the therapeutically effective amount (such as dose and dosing regimen) for such IL23 inhibitors that can be used in the combination therapy are provided in Sections 2, 4.3.2, 4.5, 4.6, 4.7 and 5; the ratio of the TL1A inhibitors and IL23 inhibitors in the combination to treat inflammatory diseases or conditions, such as IBD, UC, or CD, are provided in Sections 2 and 4.7; the pharmaceutical compositions for the TL1A inhibitors and the IL23 inhibitors in the combination therapy to treat inflammatory diseases or conditions, such as IBD, UC, or CD, are provided in Sections 4.3.1, 4.3.2, and 4.5; the methods of using the combination of TL1A inhibitors and IL23 inhibitors to treat inflammatory diseases or conditions (such as IBD, UC, or CD) are provided in Sections 2, 4.7 and 5; assays for TL1A inhibitors, IL23 inhibitors and/or for the combination of the TL1A inhibitors and IL23 inhibitors are provided in Sections 4.3.3 and 5; and specific examples of the combination of TL1A inhibitors and IL23 inhibitors and the methods of treating inflammatory diseases or conditions (such as IBD, UC, or CD) therewith are provided in Section 5. As such, the disclosure herein provides various combinations of the TL1A inhibitors, the therapeutically effective amounts (such as the dose and dosing regimens) for the TL1A inhibitors, pharmaceutical compositions for the TL1A inhibitors, the IL23 inhibitors, the therapeutically effective amounts (such as the dose and dosing regimens) for the IL23 inhibitors, and/or the pharmaceutical compositions for the IL23 inhibitors, in the methods of using the combination of TL1A inhibitors and IL23 inhibitors to treat inflammatory diseases or conditions (such as IBD, UC, or CD).

The TL1A inhibitors provided herein can have various functional properties and be of various modalities. In some embodiments, the TL1A inhibitors provided herein block, inhibit, attenuate, or reduce TL1A binding to a natural TL1A receptor, Death Receptor 3 (DR3). In some embodiments, the TL1A inhibitors provided herein block, inhibit, attenuate, or reduce TL1A-mediated signaling. In some embodiments, the TL1A inhibitors provided herein block, inhibit, attenuate, or reduce TL1A expression. In some embodiments, the TL1A inhibitors provided herein block, inhibit, attenuate, or reduce TL1A protein levels in circulation and/or diseased tissues. In some embodiments, the TL1A inhibitors provided herein block, inhibit, attenuate, or reduce TL1A activity, for example, as reflected in the mRNA transcriptome related to TL1A. In certain embodiments, the TL1A inhibitors provided herein comprise anti-TL1A antibodies or antigen-binding fragments thereof, as described in Section 4.3.1(a). In certain embodiments, the TL1A inhibitors provided herein comprise soluble DR3 protein, a variant of soluble DR3 protein, a soluble DR3 protein fused with Fc, or a variant of soluble DR3 protein fused with Fc, each as described in Section 4.3.1(c). In certain embodiments, the TL1A inhibitors provided herein comprise small molecule inhibitors of TL1A. In certain embodiments, anti-TL1A antibodies provided herein comprise inhibitory anti-TL1A antibodies or antigen binding fragments thereof as described in Section 4.3.1(a) and 4.6. In some embodiments, TL1A inhibitors provided herein comprise siRNA molecules against TL1A mRNA, as described in Section 4.3.1(b).

Similarly, IL23 inhibitors provided herein can have various functional properties and be of various modalities. In some embodiments, IL23 inhibitors comprise molecules that can block, inhibit, attenuate, or reduce IL23-mediated or IL23-dependent signaling in a cell responding to IL23. In some embodiments, IL23 inhibitors comprise molecules that can block, inhibit, attenuate, or reduce IL23 expression from IL23-expressing cells. In some embodiments, IL23 inhibitors comprise molecules that can block, inhibit, attenuate, or reduce IL23 protein levels in the subject (such as in the diseased tissue of the subject, in the blood of the subject, or other bodily fluid of the subject). In some embodiments, IL23 inhibitors comprise molecules that can block, inhibit, attenuate, or reduce IL23 binding to a natural IL23 receptor such as IL23R or the complex of an IL-23R chain and an IL-12RB1 chain. In certain embodiments, IL23 inhibitors comprise small molecule IL23 inhibitors. In certain embodiments, IL23 inhibitors comprise anti-IL23 antibodies. In certain embodiments, IL23 inhibitors comprise any IL23 inhibitor described in Section 4.3.2. In certain embodiments, IL23 inhibitors comprise inhibitory anti-IL23 antibodies or antigen binding fragments thereof. In certain embodiments, IL23 inhibitors comprise siRNA molecules against IL23 mRNA.

### 6.3.1 TL1A Inhibitors

### (a) Anti-TL1A Antibodies

TL1A exists in both monomeric and trimeric form in vivo and in vitro. The disclosure provides that although the trimeric form is the biologically active form that can bind to the physiological receptor, death receptor 3 (“DR3”) and trigger TL1A mediated signaling (e.g. Zhan, C et al., Structure 19:162-171 (2011)), monomeric TL1A accounts for a large fraction of the TL1A pool in a subject. By one of the inventors' estimates, the monomeric TL1A can be 60% of the total TL1A in the circulating blood. The term “total TL1A” refers to both monomeric and trimeric TL1A. The disclosure further provides that, despite monomeric TL1A being biologically inactive, anti-TL1A antibodies binding to both monomeric and trimeric TL1A provide advantages over antibodies binding to only trimeric TL1A. As provided herein and further demonstrated in Section 5, such advantages include more efficient reduction of the TL1A concentration in a diseased tissue in a subject including the concentration trimeric TL1A in the diseased tissue, more efficient reduction of the TL1A concentration in the blood in a subject including the concentration trimeric TL1A in the blood, more sustained reduction of TL1A concentration (including trimeric TL1A concentration) in a diseased tissue in a subject, and/or more sustained reduction of TL1A concentration (including trimeric TL1A concentration) in the blood in a subject.

In one aspect, provided herein are antibodies or antigen binding fragments thereof that bind to tumor necrosis factor-like protein 1A (“TL1A,” and such antibody or antigen binding fragment thereof, “anti-TL1A antibody or antigen binding fragment” or “anti-TL1A antibody (ies)” in the specification for simplicity), wherein the antibodies or antigen binding fragments bind to both monomeric TL1A and trimeric TL1A. Further embodiments of the anti-TL1A antibodies, including embodiments with exemplary CDRs, framework sequences, constant region sequences, Fc mutations, variable regions, Fc regions, and other properties are further provided in this Section (Section 4.3.1(a)). Assays for screening, testing, and validating the anti-TL1A antibodies are provided in Section 4.3.3. Methods for generating, improving, mutating, cloning, expressing, and isolating the anti-TL1A antibodies are provided in Section 4.4. Pharmaceutical compositions for the anti-TL1A antibodies are provided in Section 4.5. Therapeutically effective amount (such as the dose and dosing regimen) for the anti-TL1A antibodies are provided in Section 4.5 and 4.6. Methods of using the anti-TL1A antibodies in the combination therapy are provided in Section 4.7. Further specific and validated embodiments for the anti-TL1A antibodies and the methods of using the same are provided in Section 5. As such, the disclosure provides the various combinations of the anti-TL1A antibodies, the pharmaceutical compositions of such anti-TL1A antibodies, the methods of generating the anti-TL1A antibodies, the methods of assaying the anti-TL1A antibodies, and the methods of using the anti-TL1A antibodies in the combination therapy for treating an inflammatory disease and condition.

In one embodiment of the various anti-TL1A antibodies or antigen binding fragments thereof provided herein, the antibody or antigen binding fragment blocks binding of TL1A to Death Receptor 3 (“DR3”). In another embodiment, the antibody or antigen binding fragment blocks the binding of trimeric TL1A to DR3. In a further embodiment, the antibody or antigen binding fragment blocks the signaling DR3 signaling mediated by TL1A. In yet another embodiment, the antibody or antigen binding fragment blocks the increase of IFNγ secretion by various immune cells. In a specific embodiment, the antibody or antigen binding fragment blocks the increase of IFNγ secretion by peripheral blood mononuclear cells, including various B cells, T cells, natural killer cells, and/or macrophages.

As described herein, the disclosure provides anti-TL1A antibodies or antigen binding fragments for binding both monomeric and trimeric TL1A. Therefore, in one embodiment of the various anti-TL1A antibodies or antigen binding fragments thereof provided herein, binding affinity of the antibody or antigen binding fragment to monomeric TL1A as measured by dissociation equilibrium constant (K_{D-monomer}) is comparable to binding affinity of the antibody or antigen binding fragment to trimeric TL1A as measured by dissociation equilibrium constant (K_{D-trimer}). Such K_{D-monomer} and/or K_{D-trimer} can be determined via any of the methods known and practice by a skilled artisan in the field and via any of the applicable assays and methods described herein, including in this Section (Section 4.3.1(a)) and Section 5.

The relative binding affinity of the anti-TL1A antibody or antigen binding fragment for the TL1A monomer and TL1A trimer can be described and provided by K_{D-monomer} and K_{D-trimer}. In one embodiment of the various anti-TL1A antibodies or antigen binding fragments provided herein, the K_{D-monomer} is within 1.5, 2, 3, 4, 5, 6, 7, 8, 9, or 10 fold of the K_{D-trimer}. In another embodiment of the various anti-TL1A antibodies or antigen binding fragments provided herein, the K_{D-monomer} is within 10%, 20%, 30%, 40%, or 50% of the K_{D-trimer}. In a further embodiment of the various anti-TL1A antibodies or antigen binding fragments provided herein, the K_{D-trimer} is within 1.5, 2, 3, 4, 5, 6, 7, 8, 9, or 10 fold of the KD-monomer. In another embodiment of the various anti-TL1A antibodies or antigen binding fragments provided herein, the K_{D-trimer} is within 10%, 20%, 30%, 40%, or 50% of the KD-monomer.

More specifically, in one embodiment of the various anti-TL1A antibodies or antigen binding fragments provided herein, K_{D-monomer} is at most 5×10⁻¹² M, at most 6×10⁻¹² M, at most 7×10⁻¹² M, at most 8×10⁻¹² M, at most 9×10⁻¹² M, at most 1×10⁻¹¹ M, at most 2×10⁻¹¹ M, at most 3×10⁻¹¹ M, at most 4×10⁻¹¹ M, at most 5×10⁻¹¹ M, at most 6×10⁻¹¹ M, at most 7×10⁻¹¹ M, at most 8×10⁻¹¹ M, at most 9×10⁻¹¹ M, at most 1×10⁻¹⁰ M, at most 2×10⁻¹⁰ M, at most 3×10⁻¹⁰ M, at most 4×10⁻¹⁰ M, at most 5×10⁻¹⁰ M, at most 6×10⁻¹⁰ M, at most 7×10⁻¹⁰ M, at most 8×10⁻¹⁰ M, at most 9×10⁻¹⁰ M, or at most 1×10⁻⁹ M. In another embodiment, KD-monomer is about 5×10⁻¹² M, about 6×10⁻¹² M, about 7×10⁻¹² M, about 8×10⁻¹² M, about 9×10⁻¹² M, about 1×10⁻¹¹ M, about 2×10⁻¹¹ M, about 3×10⁻¹¹ M, about 4×10⁻¹¹ M, about 5×10⁻¹¹ M, about 6×10⁻¹¹ M, about 7×10⁻¹¹ M, about 8×10⁻¹¹ M, about 9×10⁻¹¹ M, about 1×10⁻¹⁰ M, about 2×10⁻¹⁰ M, about 3×10⁻¹⁰ M, about 4×10⁻¹⁰ M, about 5×10⁻¹⁰ M, about 6×10⁻¹⁰ M, about 7×10⁻¹⁰ M, about 8×10⁻¹⁰ M, about 9×10⁻¹⁰ M, or about 1×10⁻⁹ M. In a further embodiment of the various anti-TL1A antibodies or antigen binding fragments provided herein, K_{D-trimer} is at most 5×10⁻¹² M, at most 6×10⁻¹² M, at most 7×10⁻¹² M, at most 8×10⁻¹² M, at most 9×10⁻¹² M, at most 1×10⁻¹¹ M, at most 2×10⁻¹¹ M, at most 3×10⁻¹¹ M, at most 4×10⁻¹¹ M, at most 5×10⁻¹¹ M, at most 6×10⁻¹¹ M, at most 7×10⁻¹¹ M, at most 8×10⁻¹¹ M, at most 9×10⁻¹¹ M, at most 1×10⁻¹⁰ M, at most 2×10⁻¹⁰ M, at most 3×10⁻¹⁰ M, at most 4×10⁻¹⁰ M, at most 5×10⁻¹⁰ M, at most 6×10⁻¹⁰ M, at most 7×10⁻¹⁰ M, at most 8×10⁻¹⁰ M, at most 9×10⁻¹⁰ M, or at most 1×10⁻⁹ M. In yet another embodiment, K_{D-trimer} is about 5×10⁻¹² M, about 6×10⁻¹² M, about 7×10⁻¹² M, about 8×10⁻¹² M, about 9×10⁻¹² M, about 1×10⁻¹¹ M, about 2×10⁻¹¹ M, about 3×10⁻¹¹ M, about 4×10⁻¹¹ M, about 5×10⁻¹¹ M, about 6×10⁻¹¹ M, about 7×10⁻¹¹ M, about 8×10⁻¹¹ M, about 9×10⁻¹¹ M, about 1×10⁻¹⁰ M, about 2×10⁻¹⁰ M, about 3×10⁻¹⁰ M, about 4×10⁻¹⁰ M, about 5×10⁻¹⁰ M, about 6×10⁻¹⁰ M, about 7×10⁻¹⁰ M, about 8×10⁻¹⁰ M, about 9×10⁻¹⁰ M, or about 1×10⁻⁹ M. The disclosure further provides that the K_{D-monomer} and K_{D-trimer} can be any combination of the K_{D-monomer} and K_{D-trimer} value or range as provided herein, including in this Section (Section 4.3.1(a)) and this paragraph.

In a further specific embodiment, the K_{D-monomer} is about 59 pM. In another specific embodiment, the K_{D-trimer} is about 59 pM. In a further embodiment, the K_{D-monomer} is about 59 pM and the K_{D-trimer} is about 59 pM. In one specific embodiment, the K_{D-monomer} IS about 60 pM. In another specific embodiment, the K_{D-trimer} is about 60 pM. In a further embodiment, the K_{D-monomer} is about 60 pM and the K_{D-trimer} is about 60 pM. In one specific embodiment, the K_{D})-monomer is at most 60 pM. In another specific embodiment, the K_{D-trimer} is at most 60 pM. In a further embodiment, the K_{D-monomer} is at most 60 pM and the K_{D-trimer} is at most 60 pM.

In one aspect, provided herein are antibodies that bind to TL1A. In some embodiments, an antibody comprises an antigen-binding fragment that refers to a portion of an antibody having antigenic determining variable regions of an antibody. Examples of antigen-binding fragments include, but are not limited to Fab, Fab′, F(ab′)₂, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′)₂, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

In certain aspects, antibodies are described herein that specifically bind to TL1A (Entrez Gene: 9966; UniProtKB: O95150). In some embodiments, the antibodies specifically bind to soluble TL1A. In some embodiments, the antibodies specifically bind to membrane bound TL1A. In some embodiments, an anti-TL1A antibody is provided having a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. An anti-TL1A antibody may comprise any region provided herein, for example, as provided in the tables, the examples, and the sequences.

### Exemplary Anti-TL1A CDRs

In certain embodiments, an anti-TL1A antibody comprises a HCDR1 as set forth by SEQ ID NO: 1. In certain embodiments, an anti-TL1A antibody comprises a HCDR2 as set forth by any one of SEQ ID NOS: 2-5. In certain embodiments, an anti-TL1A antibody comprises a HCDR3 as set forth by any one of SEQ ID NOS: 6-9. In certain embodiments, an anti-TL1A antibody comprises a LCDR1 as set forth by SEQ ID NO: 10. In certain embodiments, an anti-TL1A antibody comprises a LCDR2 as set forth by SEQ ID NO: 11. In certain embodiments, an anti-TL1A antibody comprises a LCDR3 as set forth by any one of SEQ ID NOS: 12-15. In a non-limiting example, an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12.

In certain embodiments, an anti-TL1A antibody comprises a HCDR1, HCDR2, HCDR3, LCDR1, LCDR2, and LCDR3 selected from Table 6.

TABLE 6
Example CDR amino acid sequences

| SEQ ID NO | Description | Sequence |
|---|---|---|
| 1 | HCDR1 | GFDIQDTYMH |
| 2 | HCDR2a | RIDPASGHTKYDPKFQV |
| 3 | HCDR2b | RIEPASGHIKYDPKFQG |
| 4 | HCDR2c | RIDPASGHIKYDPKFQG |
| 5 | HCDR2d | RIEPASGHIKYDPKFQV |
| 6 | HCDR3a | SGGLPDV |
| 7 | HCDR3b | ARSGGLPDV |
| 8 | HCDR3c | SGGLPDW |
| 9 | HCDR3d | ARSGGLPDW |
| 10 | LCDR1 | RASSSVSYMY |
| 11 | LCDR2 | ATSNLAS |
| 12 | LCDR3a | QQWEGNPRT |
| 13 | LCDR3b | QQWKGNPRT |
| 14 | LCDR3c | QQWSGNPRT |
| 15 | LCDR3d | QQWSRNPRT |

In certain embodiments, an anti-TL1A antibody comprises the CDRs set forth in antibody A, B, C, D, E, F, G, H, I, A2, B2, C2, D2, E2, F2, G2, H2, or I2 of Table 10.

TABLE 10
CDR sequences from example anti-TL1A antibodies

| | Heavy Chain CDR | Light Chain CDR |
|---|---|---|
| | SEQ ID NOS | SEQ ID NOS |
| Antibody | (CDR1, CDR2, CDR3) | (CDR1, CDR2, CDR3) |
| A | 1, 2, 6 | 10, 11, 12 |
| B | 1, 3, 8 | 10, 11, 12 |
| C | 1, 4, 8 | 10, 11, 12 |
| D | 1, 2, 6 | 10, 11, 13 |
| E | 1, 2, 6 | 10, 11, 14 |
| F | 1, 5, 8 | 10, 11, 12 |
| G | 1, 5, 8 | 10, 11, 13 |
| H | 1, 3, 8 | 10, 11, 13 |
| A2 | 1, 2, 7 | 10, 11, 12 |
| B2 | 1, 3, 9 | 10, 11, 12 |
| C2 | 1, 4, 9 | 10, 11, 12 |
| D2 | 1, 2, 7 | 10, 11, 13 |
| E2 | 1, 2, 7 | 10, 11, 14 |
| F2 | 1, 5, 9 | 10, 11, 12 |
| G2 | 1, 5, 9 | 10, 11, 13 |
| H2 | 1, 3, 9 | 10, 11, 13 |
| I | 1, 5, 8 | 10, 11, 15 |
| I2 | 1, 5, 9 | 10, 11, 15 |

In certain embodiments, an anti-TL1A antibody comprises the heavy chain CDRs set forth in an antibody selected from Table 7.

TABLE 7
Example heavy chain variable region sequences

| SEQ | | |
|---|---|---|
| ID | | |
| NO | Description | Sequence |
| 101 | 217 VH, 158 VH | QVQLVQSGAEVKKPGASVKVSCKASGFDIQDTYMHWVKQRPGQGL |
| | | EWMGRIDPASGHTKYDPKFQVRVTITRDTSTSTVYMELSSLRSEDTA |
| | | VYYCARSGGLPDVWGQGTTVTVSS |
| 102 | 220 VH, 160 VH | QVQLVQSGAEVKKPGASVKVSCKASGFDIQDTYMHWVKQRPGQGL |
| | | EWMGRIDPASGHTKYDPKFQVRVTMTRDTSTSTAYLELSSLRSEDTA |
| | | VYYCARSGGLPDVWGQGTTVTVSS |
| 103 | 223 VH, 200 | EVQLVQSGAEVKKPGASVKVSCKASGFDIQDTYMHWVRQRPGQGL |
| | VH, 194 VL, 206 | EWIGRIDPASGHTKYDPKFQVRATITTDTSTSTAYLELSSLRSEDTAV |
| | VH | YYCARSGGLPDVWGQGTTVTVSS |
| 104 | 219 VH, 157 VH | QVQLVQSGAEVKKPGASVKVSCKASGFDIQDTYMHWVKQRPGQGL |
| | | EWMGRIDPASGHTKYDPKFQVRVTITRDTSTSTVYLELSSLRSEDTAV |
| | | YYCARSGGLPDVWGQGTTVTVSS |
| 105 | 221 VH, 125 VH | QVQLVQSGAEVKKPGASVKVSCKASGFDIQDTYMHWVKQRPGQGL |
| | | EWMGRIDPASGHTKYDPKFQVRATITRDTSTSTAYLELSSLRSEDTAV |
| | | YYCARSGGLPDVWGQGTTVTVSS |
| 106 | 213 VH, 162 VH | QVQLVQSGAEVKKPGASVKVSCKASGFDIQDTYMHWVRQRPGQGL |
| | | EWMGRIDPASGHTKYDPKFQVRVTITTDTSTSTVYMELSSLRSEDTA |
| | | VYYCARSGGLPDVWGQGTTVTVSS |
| 107 | 212 VH, 100 | QVQLVQSGAEVKKPGASVKVSCKASGFDIQDTYMHWVRQRPGQGL |
| | VH, 181 VH, 34 | EWIGRIDPASGHTKYDPKFQVRATITTDTSTSTAYLELSSLRSEDTAV |
| | VH, 79 VH | YYCARSGGLPDVWGQGTTVTVSS |
| 108 | 107 VH, 211 | QVQLVQSGAEVKKPGASVKVSCKASGFDIQDTYMHWVKQRPGQGL |
| | VH, 15 VH, 30 | EWIGRIDPASGHTKYDPKFQVRATITTDTSTSTAYLELSSLRSEDTAV |
| | VH, 29 VH, 48 | YYCARSGGLPDVWGQGTTVTVSS |
| | VH, 49 VH, 50 | |
| | VH, 51 VH, 52 | |
| | VH, 56 VH | |
| 109 | 205 VH, 199 | EVQLVQSGAEVKKPGASVKVSCKASGFDIQDTYMHWVKQRPGQGL |
| | VH, 55 VH, 193 | EWIGRIDPASGHTKYDPKFQVRATITTDTSTSTAYLELSSLRSEDTAV |
| | VH | YYCARSGGLPDVWGQGTTVTVSS |
| 110 | 129 VH, 139 | QVQLVQSGAEVKKPGASVKVSCKASGFDIQDTYMHWVRQRPGQGL |
| | VH, 140 VH, | EWMGRIDPASGHTKYDPKFQVRVTITIDTSTSTAYLELSSLRSEDTAV |
| | 215 VH | YYCARSGGLPDVWGQGTTVTVSS |
| 111 | 214 VH, 128 | QVQLVQSGAEVKKPGASVKVSCKASGFDIQDTYMHWVRQRPGQGL |
| | VH, 141 VH, | EWMGRIDPASGHTKYDPKFQVRVTITRDTSTSTAYLELSSLRSEDTAV |
| | 142 VH, 144 VH | YYCARSGGLPDVWGQGTTVTVSS |
| 112 | 216 VH, 123 VH | QVQLVQSGAEVKKPGASVKVSCKASGFDIQDTYMHWVRQRPGQGL |
| | | EWIGRIDPASGHTKYDPKFQVRVTITRDTSTSTAYLELSSLRSEDTAV |
| | | YYCARSGGLPDVWGQGTTVTVSS |
| 113 | 122 VH | QVQLVQSGAEVKKPGASVKVSCKASGFDIQDTYMHWVRQRPGQGL |
| | | EWIGRIDPASGHTKYDPKFQVRATITRDTSTSTAYLELSSLRSEDTAV |
| | | YYCARSGGLPDVWGQGTTVTVSS |
| 114 | 222 VH, 126 VH | QVQLVQSGAEVKKPGASVKVSCKASGFDIQDTYMHWVKQRPGQGL |
| | | EWMGRIDPASGHTKYDPKFQVRVTITRDTSTSTAYLELSSLRSEDTAV |
| | | YYCARSGGLPDVWGQGTTVTVSS |
| 115 | 188 VH, 41 VH, | QVQLVQSGAEVKKPGASVKVSCKASGFDIQDTYMHWVKQRPGQGL |
| | 102 VH | EWIGRIDPASGHTKYDPKFQVRVTITTDTSTSTAYLELSSLRSEDTAV |
| | | YYCARSGGLPDVWGQGTTVTVSS |
| 116 | 203 VH, 197 | EVQLVQSGAEVKKPGASVKVSCKASGFDIQDTYMHWVKQAPGQGL |
| | VH, 209 VH, | EWMGRIEPASGHIKYDPKFQGRVTMTRDTSTSTVYMELSSLRSEDTA |
| | 224 VH | VYYCARSGGLPDWWGQGTTVTVSS |
| 117 | 147 VH, 112 | QVQLVQSGAEVKKPGASVKVSCKASGFDIQDTYMHWVKQRPGQGL |
| | VH, 59 VH | EWMGRIEPASGHIKYDPKFQGRVTMTRDTSTSTVYMELSSLRSEDTA |
| | | VYYCARSGGLPDWWGQGTTVTVSS |
| 118 | 127 VH | QVQLVQSGAEVKKPGASVKVSCKASGFDIQDTYMHWVKQRPGQGL |
| | | EWMGRIDPASGHTKYDPKFQVRVTITTDTSTSTAYLELSSLRSEDTAV |
| | | YYCARSGGLPDVWGQGTTVTVSS |
| 119 | 159 VH, 218 VH | QVQLVQSGAEVKKPGASVKVSCKASGFDIQDTYMHWVKQRPGQGL |
| | | EWMGRIDPASGHTKYDPKFQVRVTITRDTSTSTAYMELSSLRSEDTA |
| | | VYYCARSGGLPDVWGQGTTVTVSS |
| 120 | 103 VH, 45 VH, | QVQLVQSGAEVKKPGASVKVSCKASGFDIQDTYMHWVKQRPGQGL |
| | 167 VH, 187 VH | EWIGRIDPASGHTKYDPKFQVRVTITRDTSTSTAYLELSSLRSEDTAV |
| | | YYCARSGGLPDVWGQGTTVTVSS |
| 121 | 64 VH, 148 VH, | QVQLVQSGAEVKKPGASVKVSCKASGFDIQDTYMHWVRQAPGQGL |
| | 97 VH, 114 VH, | EWMGRIEPASGHIKYDPKFQVRVTMTRDTSTSTVYMELSSLRSEDTA |
| | 130 VH, 133 | VYYCARSGGLPDWWGQGTTVTVSS |
| | VH, 137 VH, | |
| | 155 VH | |
| 122 | 67 VH, 138 VH, | QVQLVQSGAEVKKPGASVKVSCKASGFDIQDTYMHWVRQAPGQGL |
| | 115 VH, 149 | EWMGRIEPASGHIKYDPKFQVRATMTRDTSTSTVYMELSSLRSEDTA |
| | VH, 134 VH, 98 | VYYCARSGGLPDWWGQGTTVTVSS |
| | VH, 156 VH | |
| 123 | 68 VH, 99 VH, | QVQLVQSGAEVKKPGASVKVSCKASGFDIQDTYMHWVRQAPGQGL |
| | 116 VH | EWMGRIEPASGHIKYDPKFQVRVTITRDTSTSTVYMELSSLRSEDTAV |
| | | YYCARSGGLPDWWGQGTTVTVSS |
| 124 | 94 VH, 113 VH, | QVQLVQSGAEVKKPGASVKVSCKASGFDIQDTYMHWVRQAPGQGL |
| | 151 VH, 78 VH | EWMGRIEPASGHIKYDPKFQVRATITRDTSTSTVYMELSSLRSEDTAV |
| | | YYCARSGGLPDWWGQGTTVTVSS |
| 125 | 110 VH, 58 VH, | QVQLVQSGAEVKKPGASVKVSCKASGFDIQDTYMHWVKQAPGQGL |
| | 136 VH, 146 | EWMGRIEPASGHIKYDPKFQGRVTMTRDTSTSTVYMELSSLRSEDTA |
| | VH, 154 VH | VYYCARSGGLPDWWGQGTTVTVSS |
| 126 | 169 VH, 175 VH | QVQLVQSGAEVKKPGASVKVSCKASGFDIQDTYMHWVKQAPGQGL |
| | | EWMGRIDPASGHIKYDPKFQGRVTMTRDTSTSTVYMELSSLRSEDTA |
| | | VYYCARSGGLPDWWGQGTTVTVSS |
| 127 | 173 VH, 179 VH | QVQLVQSGAEVKKPGASVKVSCKASGFDIQDTYMHWVKQAPGQGL |
| | | EWMGRIEPASGHIKYDPKFQGRATMTRDTSTSTVYMELSSLRSEDTA |
| | | VYYCARSGGLPDWWGQGTTVTVSS |
| 128 | 96 VH, 132 VH, | QVQLVQSGAEVKKPGASVKVSCKASGFDIQDTYMHWVRQAPGQGL |
| | 65 VH, 150 VH | EWMGRIEPASGHIKYDPKFQGRATMTRDTSTSTVYMELSSLRSEDTA |
| | | VYYCARSGGLPDWWGQGTTVTVSS |
| 129 | 196 VH, 202 | EVQLVQSGAEVKKPGASVKVSCKASGFDIQDTYMHWVRQAPGQGL |
| | VH, 208 VH | EWMGRIEPASGHIKYDPKFQGRATMTRDTSTSTVYMELSSLRSEDTA |
| | | VYYCARSGGLPDWWGQGTTVTVSS |
| 130 | 172 VH, 178 VH | QVQLVQSGAEVKKPGASVKVSCKASGFDIQDTYMHWVRQAPGQGL |
| | | EWMGRIDPASGHIKYDPKFQGRATMTRDTSTSTVYMELSSLRSEDTA |
| | | VYYCARSGGLPDWWGQGTTVTVSS |
| 131 | 75 VH, 72 VH, | QVQLVQSGAEVKKPGASVKVSCKASGFDIQDTYMHWVRQAPGQGL |
| | 95 VH, 152 VH | EWMGRIEPASGHIKYDPKFQGRATITTDTSTSTVYMELSSLRSEDTAV |
| | | YYCARSGGLPDWWGQGTTVTVSS |
| 132 | 174 VH, 180 VH | QVQLVQSGAEVKKPGASVKVSCKASGFDIQDTYMHWVRQAPGQGL |
| | | EWMGRIEPASGHIKYDPKFQGRATMTRDTSTSTAYMELSSLRSEDTA |
| | | VYYCARSGGLPDWWGQGTTVTVSS |
| 133 | 109 VH, 91 VH, | QVQLVQSGAEVKKPGASVKVSCKASGFDIQDTYMHWVRQAPGQGL |
| | 135 VH, 145 | EWMGRIEPASGHIKYDPKFQGRVTMTRDTSTSTAYMELSSLRSEDTA |
| | VH, 153 VH | VYYCARSGGLPDWWGQGTTVTVSS |
| 134 | 198 VH, 204 | EVQLVQSGAEVKKPGASVKVSCKASGFDIQDTYMHWVRQAPGQGL |
| | VH, 210 VH | EWMGRIEPASGHIKYDPKFQGRVTMTRDTSTSTAYMELSSLRSEDTA |
| | | VYYCARSGGLPDWWGQGTTVTVSS |
| 135 | 170 VH, 176 VH | QVQLVQSGAEVKKPGASVKVSCKASGFDIQDTYMHWVRQAPGQGL |
| | | EWMGRIDPASGHIKYDPKFQGRVTMTRDTSTSTAYMELSSLRSEDTA |
| | | VYYCARSGGLPDWWGQGTTVTVSS |
| 136 | 31 VH, 85 VH, | QVQLVQSGAEVKKPGASVKVSCKASGFDIQDTYMHWVRQAPGQGL |
| | 86 VH, 87 VH, | EWMGRIEPASGHIKYDPKFQGRVTMTRDTSTSTVYMELSSLRSEDTA |
| | 88 VH, 89 VH, | VYYCARSGGLPDWWGQGTTVTVSS |
| | 90 VH, 143 VH | |
| 137 | 32 VH, 33 VH | DVQLVQSGAEVKKPGASVKVSCKASGFDIQDTYMHWVRQAPGQGL |
| | | EWMGRIEPASGHIKYDPKFQGRVTMTRDTSTSTVYMELSSLRSEDTA |
| | | VYYCARSGGLPDWWGQGTTVTVSS |
| 138 | 35 VH, 182 VH | QVQLVQSGAEVKKPGASVKVSCKASGFDIQDTYMHWVKQAPGQGL |
| | | EWIGRIDPASGHTKYDPKFQVRATITTDTSTSTAYLELSSLRSEDTAV |
| | | YYCARSGGLPDVWGQGTTVTVSS |
| 139 | 36 VH, 81 VH, | QVQLVQSGAEVKKPGASVKVSCKASGFDIQDTYMHWVRQAPGQGL |
| | 104 VH, 165 | EWIGRIDPASGHTKYDPKFQVRATITTDTSTSTAYLELSSLRSEDTAV |
| | VH, | YYCARSGGLPDVWGQGTTVTVSS |
| 140 | 37 VH, 82 VH, | QVQLVQSGAEVKKPGASVKVSCKASGFDIQDTYMHWVKQRPGQGL |
| | 101 VH, 183 VH | EWMGRIDPASGHTKYDPKFQVRATITTDTSTSTAYLELSSLRSEDTAV |
| | | YYCARSGGLPDVWGQGTTVTVSS |
| 141 | 38 VH, 190 VH | QVQLVQSGAEVKKPGASVKVSCKASGFDIQDTYMHWVKQRPGQGL |
| | | EWIGRIDPASGHTKYDPKFQVRATITTDTSTSTVYLELSSLRSEDTAV |
| | | YYCARSGGLPDVWGQGTTVTVSS |
| 142 | 39 VH, 191 VH | QVQLVQSGAEVKKPGASVKVSCKASGFDIQDTYMHWVKQRPGQGL |
| | | EWIGRIDPASGHTKYDPKFQVRATITTDTSTSTAYMELSSLRSEDTAV |
| | | YYCARSGGLPDVWGQGTTVTVSS |
| 143 | 40 VH, 105 VH, | QVQLVQSGAEVKKPGASVKVSCKASGFDIQDTYMHWVKQRPGQGL |
| | 192 VH | EWIGRIDPASGHTKYDPKFQVRATITTDTSTSTVYMELSSLRSEDTAV |
| | | YYCARSGGLPDVWGQGTTVTVSS |
| 144 | 42 VH, 83 VH, | QVQLVQSGAEVKKPGASVKVSCKASGFDIQDTYMHWVKQRPGQGL |
| | 186 VH | EWIGRIDPASGHTKYDPKFQVRATMTTDTSTSTAYLELSSLRSEDTAV |
| | | YYCARSGGLPDVWGQGTTVTVSS |
| 145 | 43 VH, 184 VH | QVQLVQSGAEVKKPGASVKVSCKASGFDIQDTYMHWVKQRPGQGL |
| | | EWIGRIDPASGHTKYDPKFQVRATITRDTSTSTAYLELSSLRSEDTAV |
| | | YYCARSGGLPDVWGQGTTVTVSS |
| 146 | 44 VH, 53 VH, | QVQLVQSGAEVKKPGASVKVSCKASGFDIQDTYMHWVKQRPGQGL |
| | 166 VH, 189 VH | EWIGRIDPASGHTKYDPKFQVRVTMTTDTSTSTAYLELSSLRSEDTAV |
| | | YYCARSGGLPDVWGQGTTVTVSS |
| 147 | 46 VH, 168 VH, | QVQLVQSGAEVKKPGASVKVSCKASGFDIQDTYMHWVKQRPGQGL |
| | 185 VH | EWIGRIDPASGHTKYDPKFQVRATMTRDTSTSTAYLELSSLRSEDTAV |
| | | YYCARSGGLPDVWGQGTTVTVSS |
| 148 | 47 VH | QVQLVQSGAEVKKPGASVKVSCKASGFDIQDTYMHWVKQRPGQGL |
| | | EWIGRIDPASGHTKYDPKFQVRVTMTRDTSTSTAYLELSSLRSEDTAV |
| | | YYCARSGGLPDVWGQGTTVTVSS |
| 149 | 54 VH | DVQLVQSGAEVKKPGASVKVSCKASGFDIQDTYMHWVKQRPGQGL |
| | | EWIGRIDPASGHTKYDPKFQVRATITTDTSTSTAYLELSSLRSEDTAV |
| | | YYCARSGGLPDVWGQGTTVTVSS |
| 150 | 57 VH, 111 VH | QVQLVQSGAEVKKPGASVKVSCKASGFDIQDTYMHWVRQRPGQGL |
| | | EWMGRIEPASGHIKYDPKFQGRVTMTRDTSTSTVYMELSSLRSEDTA |
| | | VYYCARSGGLPDWWGQGTTVTVSS |
| 151 | 60 VH, 117 VH | QVQLVQSGAEVKKPGASVKVSCKASGFDIQDTYMHWVRQAPGQGL |
| | | EWMGRIDPASGHIKYDPKFQGRVTMTRDTSTSTVYMELSSLRSEDTA |
| | | VYYCARSGGLPDWWGQGTTVTVSS |
| 152 | 61 VH | QVQLVQSGAEVKKPGASVKVSCKASGFDIQDTYMHWVRQAPGQGL |
| | | EWIGRIEPASGHIKYDPKFQGRVTMTRDTSTSTVYMELSSLRSEDTAV |
| | | YYCARSGGLPDWWGQGTTVTVSS |
| 153 | 62 VH, 118 VH | QVQLVQSGAEVKKPGASVKVSCKASGFDIQDTYMHWVRQAPGQGL |
| | | EWIGRIDPASGHIKYDPKFQGRVTMTRDTSTSTVYMELSSLRSEDTAV |
| | | YYCARSGGLPDWWGQGTTVTVSS |
| 154 | 63 VH, 120 VH | QVQLVQSGAEVKKPGASVKVSCKASGFDIQDTYMHWVRQAPGQGL |
| | | EWMGRIEPASGHVKYDPKFQGRVTMTRDTSTSTVYMELSSLRSEDT |
| | | AVYYCARSGGLPDWWGQGTTVTVSS |
| 155 | 66 VH | QVQLVQSGAEVKKPGASVKVSCKASGFDIQDTYMHWVRQAPGQGL |
| | | EWMGRIEPASGHIKYDPKFQGRVTITRDTSTSTVYMELSSLRSEDTAV |
| | | YYCARSGGLPDWWGQGTTVTVSS |
| 156 | 69 VH, 108 VH | EVQLVQSGAEVKKPGASVKVSCKASGFDIQDTYMHWVRQAPGQGL |
| | | EWMGRIEPASGHIKYDPKFQGRVTMTRDTSTSTVYMELSSLRSEDTA |
| | | VYYCARSGGLPDWWGQGTTVTVSS |
| 157 | 70 VH, 73 VH | QVQLVQSGAEVKKPGASVKVSCKASGFDIQDTYMHWVRQAPGQGL |
| | | EWMGRIEPASGHIKYDPKFQGRVTMTTDTSTSTVYMELSSLRSEDTA |
| | | VYYCARSGGLPDWWGQGTTVTVSS |
| 158 | 71 VH, 74 VH | QVQLVQSGAEVKKPGASVKVSCKASGFDIQDTYMHWVRQAPGQGL |
| | | EWMGRIEPASGHIKYDPKFQGRVTITTDTSTSTVYMELSSLRSEDTAV |
| | | YYCARSGGLPDWWGQGTTVTVSS |
| 159 | 76 VH, 119 VH | QVQLVQSGAEVKKPGASVKVSCKASGFDIQDTYMHWVRQAPGQGL |
| | | EWMGRIEPASGHTKYDPKFQGRVTMTRDTSTSTVYMELSSLRSEDTA |
| | | VYYCARSGGLPDWWGQGTTVTVSS |
| 160 | 77 VH | QVQLVQSGAEVKKPGASVKVSCKASGFDIQDTYMHWVRQAPGQGL |
| | | EWMGRIEPASGHIKYDPKFQGRATITRDTSTSTVYMELSSLRSEDTAV |
| | | YYCARSGGLPDWWGQGTTVTVSS |
| 161 | 92 VH | QVQLVQSGAEVKKPGASVKVSCKASGFDIQDTYMHWVRQAPGQGL |
| | | EWMGRIEPASGHIKYDPKFQGRVTMTRDTSTSTVYLELSSLRSEDTA |
| | | VYYCARSGGLPDWWGQGTTVTVSS |
| 162 | 93 VH | QVQLVQSGAEVKKPGASVKVSCKASGFDIQDTYMHWVRQAPGQGL |
| | | EWMGRIEPASGHIKYDPKFQGRVTMTRDTSTSTAYLELSSLRSEDTA |
| | | VYYCARSGGLPDWWGQGTTVTVSS |
| 163 | 121 VH | QVQLVQSGAEVKKPGASVKVSCKASGFDIQDTYMHWVRQRPGQGL |
| | | EWMGRIDPASGHTKYDPKFQVRATITTDTSTSTAYLELSSLRSEDTAV |
| | | YYCARSGGLPDVWGQGTTVTVSS |
| 164 | 124 VH | QVQLVQSGAEVKKPGASVKVSCKASGFDIQDTYMHWVRQRPGQGL |
| | | EWIGRIDPASGHTKYDPKFQVRVTITTDTSTSTAYLELSSLRSEDTAV |
| | | YYCARSGGLPDVWGQGTTVTVSS |
| 165 | 161 VH | QVQLVQSGAEVKKPGASVKVSCKASGFDIQDTYMHWVRQRPGQGL |
| | | EWMGRIDPASGHTKYDPKFQVRVTITTDTSTSTVYLELSSLRSEDTAV |
| | | YYCARSGGLPDVWGQGTTVTVSS |
| 166 | 163 VH | QVQLVQSGAEVKKPGASVKVSCKASGFDIQDTYMHWVRQRPGQGL |
| | | EWMGRIDPASGHTKYDPKFQVRVTITTDTSTSTAYMELSSLRSEDTA |
| | | VYYCARSGGLPDVWGQGTTVTVSS |
| 167 | 164 VH | QVQLVQSGAEVKKPGASVKVSCKASGFDIQDTYMHWVRQRPGQGL |
| | | EWMGRIDPASGHTKYDPKFQVRVTMTTDTSTSTAYLELSSLRSEDTA |
| | | VYYCARSGGLPDVWGQGTTVTVSS |
| 168 | 171 VH, 177 VH | QVQLVQSGAEVKKPGASVKVSCKASGFDIQDTYMHWVRQAPGQGL |
| | | EWMGRIDPASGHIKYDPKFQGRATITTDTSTSTVYMELSSLRSEDTAV |
| | | YYCARSGGLPDWWGQGTTVTVSS |
| 169 | 195 VH, 201 | EVQLVQSGAEVKKPGASVKVSCKASGFDIQDTYMHWVRQAPGQGL |
| | VH, 207 VH | EWMGRIEPASGHIKYDPKFQGRATITTDTSTSTVYMELSSLRSEDTAV |
| | | YYCARSGGLPDWWGQGTTVTVSS |

In certain embodiments, an anti-TL1A antibody comprises the light chain CDRs set forth in an antibody selected from Table 8.

TABLE 8
Example light chain variable region sequences

| SEQ | | |
|---|---|---|
| ID | | |
| NO | Description | Sequence |
| 201 | 217 VL, | EIVLTQSPGTLSLSPGERATLSCRASSSVSYMYWYQQ |
| | 219 VL, 221 VL, | KPGQAPRPLIYATSNLASGIPDRFSGSGSGTDFTLTIS |
| | 200 VL, 213 VL, | RLEPEDFAVYYCQQWEGNPRTFGGGTKLEIK |
| | 212 VL, 211 VL, | |
| | 199 VL, 214 VL, | |
| | 216 VL, 222 VL, | |
| | 203 VL, 147 VL, | |
| | 218 VL, 179 VL, | |
| | 148 VL, 149 VL, | |
| | 151 VL, 180 VL, 175 VL, | |
| | 178 VL, 145 VL, 146 VL, | |
| | 150 VL, 152 VL, 176 VL, | |
| | 177 VL, 201 VL, 202 VL, | |
| | 204 VL, 215 VL, 224 VL | |
| 202 | 223 VL, 107 VL, | EIVLTQSPGTLSLSPGERATLSCRASSSVSYMYWYQQ |
| | 205 VL, 181 VL, | KPGQAPRLLIYATSNLASGIPDRFSGSGSGTDFTLTIS |
| | 188 VL, 64 VL, | RLEPEDFAVYYCQQWEGNPRTFGGGTKLEIK |
| | 67 VL, 68 VL, | |
| | 94 VL, 33 VL, 57 VL, 58 | |
| | VL, 59 VL, 60 VL, 61 VL, | |
| | 62 VL, 63 VL, 65 VL, 66 | |
| | VL, 69 VL, 70 VL, 71 VL, | |
| | 72 VL, 76 VL, 77 VL, 78 | |
| | VL, 91 VL, 92 VL, 93 VL, | |
| | 97 VL, 98 VL, 99 VL, 140 | |
| | VL, 142 VL, 143 VL, 182 | |
| | VL, 183 VL, 184 VL, 185 | |
| | VL, 186 VL, 187 VL, 189 | |
| | VL, 190 VL, 191 VL, 192 | |
| | VL, 206 VL, 207 VL, 208 | |
| | VL, 209 VL, 210 VL | |
| 203 | 15 VL | EIVLTQSPGTLSLSPGERATLSCRASSSVSYMYWYQQ |
| | | KPGQAPRLLIYATSNLASGIPDRFSGSGSGTDFTLTIS |
| | | RLEPEDFAVYYCQQWSGNPRTFGGGTKLEIK |
| 204 | 30 VL, 100 VL, | EIVLTQSPGTLSLSPGERATLSCRASSSVSYMYWYQQ |
| | 129 VL, 122 VL, | KPGQAPRLLIYATSNLASGIPDRFSGSGSGTDFTLTIS |
| | 127 VL, 126 VL, | RLEPEDFAVYYCQQWKGNPRTFGGGTKLEIK |
| | 160 VL, 157 VL, | |
| | 159 VL, 158 VL, | |
| | 125 VL, 103 VL, 101 VL, | |
| | 102 VL, 104 VL, 105 VL, | |
| | 121 VL, 123 VL, 124 VL, | |
| | 128 VL, 144 VL, 161 VL, | |
| | 162 VL, 163 VL, 164 VL | |
| 205 | 110 VL, 197 VL, | EIVLTQSPGTLSLSPGERATLSCRASSSVSYMYWYQQ |
| | 112 VL, 169 VL, | KPGQAPRPWIYATSNLASGIPDRFSGSGSGTDFTLTIS |
| | 173 VL, 115 VL, | RLEPEDFAVYYCQQWEGNPRTFGGGTKLEIK |
| | 113 VL, 96 VL, | |
| | 196 VL, 172 VL, | |
| | 75 VL, 174 VL, | |
| | 109 VL, 198 VL, | |
| | 170 VL, 29 VL, 31 VL, 32 | |
| | VL, 73 VL, 74 VL, 95 VL, | |
| | 108 VL, 111 VL, 114 VL, | |
| | 116 VL, 117 VL, 118 VL, | |
| | 119 VL, 120 VL, 130 VL, | |
| | 153 VL, 154 VL, 155 VL, | |
| | 156 VL, 165 VL, 166 VL, | |
| | 167 VL, 168 VL, 171 VL, | |
| | 193 VL, 194 VL, 195 VL, | |
| | 220 VL | |
| 206 | 134 VL, 132 VL, 133 VL, | EIVLTQSPGTLSLSPGERATLSCRASSSVSYMYWYQQ |
| | 135 VL, 136 VL | KPGQAPRPLIYATSNLASGIPDRFSGSGSGTDFTLTIS |
| | | RLEPEDFAVYYCQQWKGNPRTFGGGTKLEIK |
| 207 | 138 VL, 137 VL, 139 VL, | EIVLTQSPGTLSLSPGERATLSCRASSSVSYMYWYQQ |
| | 141 VL | KPGQAPRLLIYATSNLASGIPDRFSGSGSGTDFTLTIS |
| | | RLEPEDFAVYYCQQWSRNPRTFGGGTKLEIK |
| 208 | 34 VL, 35 VL, 36 VL, 37 | EIVLTQSPGTLSASPGERATMSCRASSSVSYMYWYQ |
| | VL, 38 VL, 39 VL, 40 VL, | QKPGQAPRPWIYATSNLASGVPDRFSGSGSGTDYTL |
| | 41 VL, 42 VL, 43 VL, 44 | TISRVEPEDFAVYYCQQWSGNPRTFGGGTKLEIK |
| | VL, 45 VL, 46 VL, 47 VL, | |
| | 53 VL, 54 VL, 55 VL, 79 | |
| | VL, 81 VL, 82 VL, 83 VL | |
| 209 | 85 VL | EIVLTQSPGTLSLSPGERATLSCRASSSVSYMYWYQQ |
| | | KPGQAPRLLIYATSNLASGVPDRFSGSGSGTDFTLTIS |
| | | RLEPEDFAVYYCQQWEGNPRTFGGGTKLEIK |
| 210 | 48 VL | EIVLTQSPGTLSASPGERATLSCRASSSVSYMYWYQ |
| | | QKPGQAPRPWIYATSNLASGVPDRFSGSGSGTDYTL |
| | | TISRVEPEDFAVYYCQQWSGNPRTFGGGTKLEIK |
| 211 | 49 VL | EIVLTQSPGTLSASPGERATMSCRASSSVSYMYWYQ |
| | | QKPGQAPRLLIYATSNLASGVPDRFSGSGSGTDYTLT |
| | | ISRVEPEDFAVYYCQQWSGNPRTFGGGTKLEIK |
| 212 | 50 VL | EIVLTQSPGTLSASPGERATMSCRASSSVSYMYWYQ |
| | | QKPGQAPRPWIYATSNLASGVPDRFSGSGSGTDFTLT |
| | | ISRVEPEDFAVYYCQQWSGNPRTFGGGTKLEIK |
| 213 | 51 VL | EIVLTQSPGTLSASPGERATMSCRASSSVSYMYWYQ |
| | | QKPGQAPRPWIYATSNLASGVPDRFSGSGSGTDYTL |
| | | TISRLEPEDFAVYYCQQWSGNPRTFGGGTKLEIK |
| 214 | 52 VL | EIVLTQSPGTLSASPGERATMSCRASSSVSYMYWYQ |
| | | QKPGQAPRPWIYATSNLASGVPDRFSGSGSGTDFTLT |
| | | ISRLEPEDFAVYYCQQWSGNPRTFGGGTKLEIK |
| 215 | 56 VL | EIVLTQSPGTLSASPGERATMSCRASSSVSYMYWYQ |
| | | QKPGQAPRPWIYATSNLASGIPDRFSGSGSGTDYTLT |
| | | ISRVEPEDFAVYYCQQWSGNPRTFGGGTKLEIK |
| 216 | 86 VL | EIVLTQSPGTLSLSPGERATLSCRASSSVSYMYWYQQ |
| | | KPGQAPRLLIYATSNLASGIPDRFSGSGSGTDYTLTIS |
| | | RLEPEDFAVYYCQQWEGNPRTFGGGTKLEIK |
| 217 | 87 VL | EIVLTQSPGTLSLSPGERATLSCRASSSVSYMYWYQQ |
| | | KPGQAPRLLIYATSNLASGIPDRFSGSGSGTDFTLTIS |
| | | RVEPEDFAVYYCQQWEGNPRTFGGGTKLEIK |
| 218 | 88 VL | EIVLTQSPGTLSLSPGERATLSCRASSSVSYMYWYQQ |
| | | KPGQAPRLLIYATSNLASGIPDRFSGSGSGTDYTLTIS |
| | | RVEPEDFAVYYCQQWEGNPRTFGGGTKLEIK |
| 219 | 89 VL | EIVLTQSPGTLSASPGERATLSCRASSSVSYMYWYQ |
| | | QKPGQAPRLLIYATSNLASGIPDRFSGSGSGTDFTLTI |
| | | SRLEPEDFAVYYCQQWEGNPRTFGGGTKLEIK |
| 220 | 90 VL | EIVLTQSPGTMSLSPGERATLSCRASSSVSYMYWYQ |
| | | QKPGQAPRLLIYATSNLASGIPDRFSGSGSGTDFTLTI |
| | | SRLEPEDFAVYYCQQWEGNPRTFGGGTKLEIK |

In certain embodiments, an anti-TL1A antibody comprises the CDRs set forth in any one of the antibodies of Table 1. For instance, an anti-TL1A antibody comprises the CDRs of antibody A15, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A81, A82, A83, A85, A86, A87, A88, A89, A90, A91, A92, A93, A94, A95, A96, A97, A98, A99, A100, A101, A102, A103, A104, A105, A107, A108, A109, A110, A111, A112, A113, A114, A115, A116, A117, A118, A119, A120, A121, A122, A123, A124, A125, A126, A127, A128, A129, A130, A132, A133, A134, A135, A136, A137, A138, A139, A140, A141, A142, A143, A144, A145, A146, A147, A148, A149, A150, A151, A152, A153, A154, A155, A156, A157, A158, A159, A160, A161, A162, A163, A164, A165, A166, A167, A168, A169, A170, A171, A172, A173, A174, A175, A176, A177, A178, A179, A180, A181, A182, A183, A184, A185, A186, A187, A188, A189, A190, A191, A192, A193, A194, A195, A196, A197, A198, A199, A200, A201, A202, A203, A204, A205, A206, A207, A208, A209, A210, A211, A212, A213, A214, A215, A216, A217, A218, A219, A220, A221, A222, A223, A224, A500, or A501. In a non-limiting example, an anti-TL1A antibody comprises the CDRs of antibody A219.

Antibody CDRs may be defined by the Aho or Kabat, Chothia, or IMGT methods.

### Exemplary Anti-TL1A Framework Regions

In certain embodiments, an anti-TL1A antibody comprises a heavy chain (HC) framework 1 (FR1) as set forth by SEQ ID NO: 304. In certain embodiments, an anti-TL1A antibody comprises a HC FR2 as set forth by any one of SEQ ID NOS: 305 or 313. In certain embodiments, an anti-TL1A antibody comprises a HC FR3 as set forth by any one of SEQ ID NOS: 306-307, 314-315. In certain embodiments, an anti-TL1A antibody comprises a HC FR4 as set forth by SEQ ID NO: 308. In certain embodiments, an anti-TL1A antibody comprises a LC FR1 as set forth by SEQ ID NO: 309. In certain embodiments, an anti-TL1A antibody comprises a LC FR2 as set forth by SEQ ID NO: 310. In certain embodiments, an anti-TL1A antibody comprises a LC FR3 as set forth by SEQ ID NO: 311. In certain embodiments, an anti-TL1A antibody comprises a LC FR4 as set forth by SEQ ID NO: 312. In a non-limiting example, an anti-TL1A antibody comprises a HC FR1 as set forth by SEQ ID NO: 304, a HC FR2 as set forth by SEQ ID NO: 305, a HC FR3 as set forth by SEQ ID NO: 306, a HC FR4 as set forth by SEQ ID NO: 308, a LC FR1 as set forth by SEQ ID NO: 309, a LC FR2 as set forth by SEQ ID NO: 310, a LC FR3 as set forth by SEQ ID NO: 311, and a LC FR4 as set forth by SEQ ID NO: 312. In a non-limiting example, an anti-TL1A antibody comprises a HC FR1 as set forth by SEQ ID NO: 304, a HC FR2 as set forth by SEQ ID NO: 305, a HC FR3 as set forth by SEQ ID NO: 307, a HC FR4 as set forth by SEQ ID NO: 308, a LC FR1 as set forth by SEQ ID NO: 309, a LC FR2 as set forth by SEQ ID NO: 310, a LC FR3 as set forth by SEQ ID NO: 311, and a LC FR4 as set forth by SEQ ID NO: 312.

In certain embodiments, an anti-TL1A antibody comprises the heavy chain framework regions set forth in an antibody selected from Table 7. In certain embodiments, an anti-TL1A antibody comprises the light chain framework regions set forth in an antibody selected from Table 8. In certain embodiments, an anti-TL1A antibody comprises the framework regions set forth in any one of the antibodies of Table 1. For instance, an anti-TL1A antibody comprises the framework regions of antibody A15, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A81, A82, A83, A85, A86, A87, A88, A89, A90, A91, A92, A93, A94, A95, A96, A97, A98, A99, A100, A101, A102, A103, A104, A105, A107, A108, A109, A110, A111, A112, A113, A114, A115, A116, A117, A118, A119, A120, A121, A122, A123, A124, A125, A126, A127, A128, A129, A130, A132, A133, A134, A135, A136, A137, A138, A139, A140, A141, A142, A143, A144, A145, A146, A147, A148, A149, A150, A151, A152, A153, A154, A155, A156, A157, A158, A159, A160, A161, A162, A163, A164, A165, A166, A167, A168, A169, A170, A171, A172, A173, A174, A175, A176, A177, A178, A179, A180, A181, A182, A183, A184, A185, A186, A187, A188, A189, A190, A191, A192, A193, A194, A195, A196, A197, A198, A199, A200, A201, A202, A203, A204, A205, A206, A207, A208, A209, A210, A211, A212, A213, A214, A215, A216, A217, A218, A219, A220, A221, A222, A223, A224, A500, or A501. In a non-limiting example, an anti-TL1A antibody comprises the framework region of antibody A219.

Antibody CDR and framework regions may be defined by the Aho or Kabat, Chothia, or IMGT methods.

In some embodiments, an anti-TL1A antibody comprises a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise no or fewer than nine amino acid modification(s) from the human IGHV1-46*02 framework and the human IGKV3-20 framework. In some embodiments, the amino acid modification(s) comprise: (a) a modification at amino acid position 45 in the heavy chain variable region; (b) a modification at amino acid position 47 in the heavy chain variable region; (c) a modification at amino acid position 55 in the heavy chain variable region; (d) a modification at amino acid position 78 in the heavy chain variable region; (e) a modification at amino acid position 80 in the heavy chain variable region; (f) a modification at amino acid position 82 in the heavy chain variable region; (g) a modification at amino acid position 89 in the heavy chain variable region; or (h) a modification at amino acid position 91 in the heavy chain variable region, per Aho or Kabat numbering; or a combination of two or more modifications selected from (a) to (h). In some embodiments, the amino acid modification(s) comprise (a) R45K, (b) A47R, (c) M55I, (d) V78A, (e) M80I, (f) R82T, (g) V89A, or (h) M91L in the heavy chain variable region, per Aho or Kabat numbering; or a combination of two or more modifications selected from (a) to (h). In some embodiments, the amino acid modification(s) comprise: A47R. In some embodiments, the amino acid modification(s) comprise: A47R, M55I, V78A, M80I, R82T, V89A, and M91L; A47R, M80I, and R82T; A47R, M80I, R82T, V89A, and M91L; or A47R, M55I, V78A, M80I, V89A, and M91L. In some embodiments, the amino acid modification(s) comprise: R45K and A47R. In some embodiments, the amino acid modification(s) comprise: R45K, A47R, V89A, and M91L. In some embodiments, the amino acid modification(s) comprise: R45K and A47R, and M80I. In some embodiments, the amino acid modification(s) comprise: R45K, A47R, M80I, and M91L; R45K, A47R, V78A, M80I, V89A, and M91L; R45K, A47R, M55I, V78A, M80I, R82T, V89A, and M91L; R45K, A47R, M80I, V89A, and M91L; R45K, A47R, M55I, M80I, R82T, V89A, and M91L; R45K, A47R, M80I, and V89A; R45K, A47R, M80I, R82T, V89A, M91L; or R45K, A47R, M55I, M80I, V89A, and M91L. In some embodiments, the amino acid modification(s) comprise: R45K. In some embodiments, the amino acid modification(s) comprise: R45K and V78A. In some embodiments, the amino acid modification(s) comprise: V78A. In some embodiments, the amino acid modification(s) comprise: V78A and V89A; V78A and M80I; or V78A, M80I, and R82T. In some embodiments, the amino acid modification(s) comprise: V89A. In some embodiments, the amino acid modification(s) comprise: M80I. In some embodiments, the amino acid modification(s) comprises: (a) a modification at amino acid position 54 in the light chain variable region; and/or (b) a modification at amino acid position 55 in the light chain variable region, per Aho or Kabat numbering. In some embodiments, the amino acid modification(s) comprises L54P in the light chain variable region, per Aho or Kabat numbering. In some embodiments, the amino acid modification(s) comprises L55W in the light chain variable region, per Aho or Kabat numbering.

In some embodiments, an anti-TL1A antibody comprises a heavy chain framework comprising SEQ ID NO: 301 (X1VQLVQSGAEVKKPGASVKVSCKAS[HCDR1]WVX2QX3PGQGLEWX4G[HCDR2]RX5TX6TX7DTSTSTX8YX9ELSSLRSEDTAVYYCAR[HCDR3]WGQGTTVTVSS) or SEQ ID NO: 302 (X1VQLVQSGAEVKKPGASVKVSCKAS[HCDR1]WVX2QX3PGQGLEWX4G[HCDR2]RX5TX6TX7DTSTSTX8YX9ELSSLRSEDTAVYYC[HCDR3]WGQGTTVTVSS). In some cases, X1 is Q. In some cases, X1=E. In some cases, X2=R. In some cases, X2=K. In some cases, X3=A. In some cases, X3=R. In some cases, X4=M. In some cases, X4=I. In some cases, X5=V. In some cases, X5=A. In some cases, X6=M. In some cases, X6=I. In some cases, X7=R. In some cases, X7=T. In some cases, X8=V. In some cases, X8=A. In some cases, X9=M. In some cases, X9=L. In some embodiments, X1 is at position 1 of IGHV1-46*02 as determined by Aho or Kabat numbering. In some embodiments, X2 is at position 45 of IGHV1-46*02 as determined by Aho or Kabat numbering. In some embodiments, X3 is at position 47 of IGHV1-46*02 as determined by Aho or Kabat numbering. In some embodiments, X4 is at position 55 of IGHV1-46*02 as determined by Aho or Kabat numbering. In some embodiments, X5 is at position 78 of IGHV1-46*02 as determined by Aho or Kabat numbering. In some embodiments, X6 is at position 80 of IGHV1-46*02 as determined by Aho or Kabat numbering. In some embodiments, X7 is at position 82 of IGHV1-46*02 as determined by Aho or Kabat numbering. In some embodiments, X8 is at position 89 of IGHV1-46*02 as determined by Aho or Kabat numbering. In some embodiments, X9 is at position 91 of IGHV1-46*02 as determined by Aho or Kabat numbering.

In one aspect, provided herein is a first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 301. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X11 is L. (38) The anti-TL1A antibody of any one of embodiments 34-36, wherein X11 is W.

In some embodiments, an anti-TL1A antibody comprises a light chain framework comprising SEQ ID NO: 303 (EIVLTQSPGTLSLSPGERATLSC[LCDR1]WYQQKPGQAPRX10X11IY[LCDR2]GIPDR FSGSGSGTDFTLTISRLEPEDFAVYYC[LCDR3]FGGGTKLEIK). In some cases, X10 is L. In some cases, X10 is P. In some cases, X11 is L. In some cases, X11 is W. In some embodiments, X10 is at position 54 of IGKV3-20*01 as determined by Aho or Kabat numbering. In some embodiments, X11 is at position 55 of IGKV3-20*01 as determined by Aho or Kabat numbering.

In some embodiments, an anti-TL1A antibody comprises a heavy chain framework comprising IGHV1-46*02. In some embodiments, an anti-TL1A antibody comprises a heavy chain framework comprising a variant of IGHV1-46*02 comprising between about 1 and about 20 amino acid substitutions from SEQ ID NO: 316. In some embodiments, an anti-TL1A antibody comprises a heavy chain framework comprising a variant of IGHV1-46*02 comprising between about 1 and about 9 amino acid substitutions from SEQ ID NO: 316. In some embodiments, an anti-TL1A antibody comprises a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. In some cases, the heavy chain framework substitution comprises Q1E, as determined by Aho or Kabat numbering. In some cases, the heavy chain framework substitution comprises R45K, as determined by Aho or Kabat numbering. In some cases, the heavy chain framework substitution comprises A47R, as determined by Aho or Kabat numbering. In some cases, the heavy chain framework substitution comprises M55I, as determined by Aho or Kabat numbering. In some cases, the heavy chain framework substitution comprises V78A, as determined by Aho or Kabat numbering. In some cases, the heavy chain framework substitution comprises M80I, as determined by Aho or Kabat numbering. In some cases, the heavy chain framework substitution comprises R82T, as determined by Aho or Kabat numbering. In some cases, the heavy chain framework substitution comprises V89A, as determined by Aho or Kabat numbering. In some cases, the heavy chain framework substitution comprises M91L, as determined by Aho or Kabat numbering.

In some embodiments, an anti-TL1A antibody comprises a light chain framework comprising IGKV3-20*01. In some embodiments, an anti-TL1A antibody comprises a variant of IGKV3-20*01 comprising between about 1 and about 20 amino acid substitutions from SEQ ID NO: 317. In some embodiments, an anti-TL1A antibody comprises a variant of IGKV3-20*01 comprising about 1 amino acid substitution from SEQ ID NO: 317. In some embodiments, an anti-TL1A antibody comprises a light chain framework comprising a variant of IGKV3-20*01 comprising about 2 amino acid substitutions from SEQ ID NO: 317. In some embodiments, an anti-TL1A antibody comprises a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. In some cases, the light chain framework substitution comprises Q1E, as determined by Aho or Kabat numbering. In some cases, the light chain framework substitution comprises R45K, as determined by Aho or Kabat numbering.

In some embodiments, an anti-TL1A antibody comprises a heavy chain FR1 as set forth by SEQ ID NO: 304. In some embodiments, an anti-TL1A antibody comprises a heavy chain FR2 as set forth by SEQ ID NO: 305. In some embodiments, an anti-TL1A antibody comprises a heavy chain FR2 as set forth by SEQ ID NO: 313. In some embodiments, an anti-TL1A antibody comprises a heavy chain FR3 as set forth by SEQ ID NO: 306. In some embodiments, an anti-TL1A antibody comprises a heavy chain FR3 as set forth by SEQ ID NO: 307. In some embodiments, an anti-TL1A antibody comprises a heavy chain FR3 as set forth by SEQ ID NO: 314. In some embodiments, an anti-TL1A antibody comprises a heavy chain FR3 as set forth by SEQ ID NO: 315. In some embodiments, an anti-TL1A antibody comprises a heavy chain FR4 as set forth by SEQ ID NO: 308. In some embodiments, an anti-TL1A antibody comprises a light chain FR1 as set forth by SEQ ID NO: 309. In some embodiments, an anti-TL1A antibody comprises a light chain FR2 as set forth by SEQ ID NO: 310. In some embodiments, an anti-TL1A antibody comprises a light chain FR3 as set forth by SEQ ID NO: 311. In some embodiments, an anti-TL1A antibody comprises a light chain FR4 as set forth by SEQ ID NO: 312.

In some embodiments, an anti-TL1A antibody comprises a framework region of Table 9A.

TABLE 9A
Example framework sequences

| SEQ | | |
|---|---|---|
| ID | | |
| NO | Description | Sequence |
| 301 | Variable Heavy 1 | X1VQLVQSGAEVKKPGASVKVSCKAS[HCDR1]WVX2QX3PGQG |
| | | LEWX4G[HCDR2]RX5TX6TX7DTSTSTX8YX9ELSSLRSEDTA |
| | | VYYCAR[HCDR3]WGQGTTVTVSS |
| | | wherein each of X1-X11 is independently selected from A, R, N, |
| | | D, C, Q, E, G, H, I, L, K, M, F, P, S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 302 | Variable Heavy 2 | X1VQLVQSGAEVKKPGASVKVSCKAS[HCDR1]WVX2QX3PGQG |
| | | LEWX4G[HCDR2]RX5TX6TX7DTSTSTX8YX9ELSSLRSEDTA |
| | | VYYC[HCDR3]WGQGTTVTVSS |
| | | wherein each of X1-X11 is independently selected from A, R, N, |
| | | D, C, Q, E, G, H, I, L, K, M, F, P, S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 303 | Variable Light | EIVLTQSPGTLSLSPGERATLSC[LCDR1]WYQQKPGQAPRX10 |
| | | X11IY[LCDR2]GIPDRFSGSGSGTDFTLTISRLEPEDFAVYYC |
| | | [LCDR3]FGGGTKLEIK |
| | | wherein each of X10 and X11 is independently selected from A, |
| | | R, N, D, C, Q, E, G, H, I, L, K, M, F, P, S, T, W, Y, or V |
| | | In some cases, X10 = L or P |
| | | In some cases, X11 = L or W |
| 304 | 219 HC FR1, | QVQLVQSGAEVKKPGASVKVSCKAS |
| | 212 HC FR1 | |
| 305 | 219 HC FR2 | WVKQRPGQGLEWMG |
| 306 | 219 HC FR3a | RVTITRDTSTSTVYLELSSLRSEDTAVYYCAR |
| 307 | 219 HC FR3b | RVTITRDTSTSTVYLELSSLRSEDTAVYYC |
| 308 | 219 HC FR4, | WGQGTTVTVSS |
| | 212 HC FR4 | |
| 309 | 219 LC FR1, | EIVLTQSPGTLSLSPGERATLSC |
| | 212 LC FR1 | |
| 310 | 219 LC FR2, | WYQQKPGQAPRPLIY |
| | 212 LC FR2 | |
| 311 | 219 LC FR3, | GIPDRFSGSGSGTDFTLTISRLEPEDFAVYYC |
| | 212 LC FR3 | |
| 312 | 219 LC FR4, | FGGGTKLEIK |
| | 212 LC FR4 | |
| 313 | 212 HC FR2 | WVRQRPGQGLEWIG |
| 314 | 212 HC FR3a | RATITTDTSTSTAYLELSSLRSEDTAVYYCAR |
| 315 | 212 HC FR3b | RATITTDTSTSTAYLELSSLRSEDTAVYYC |
| 316 | IGHV1-46*02 | QVQLVQSGAEVKKPGASVKVSCKASGYTFNSYYMHWVR |
| | | QAPGQGLEWMGIINPSGGSTSYAQKFQGRVTMTRDTSTST |
| | | VYMELSSLRSEDTAVYYCAR |
| 317 | IGKV3-20*01 | EIVLTQSPGTLSLSPGERATLSCRASQSVSSSYLAWYQQKPG |
| | | QAPRLLIYGASSRATGIPDRFSGSGSGTDFTLTISRLEPEDFA |
| | | VYYCQQYGSSP |

### Exemplary Anti-TL1A Variable Regions

In one aspect, provided herein is an anti-TL1A antibody comprising a heavy chain variable region comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 101-169; and a light chain variable region at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 201-220.

Further provided herein is a first embodiment of an anti-TL1A antibody comprising a heavy chain variable region and a light chain variable region. Non-limiting additional embodiments include: (Embodiment 2) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 101 or a sequence having about 1, 2, 3, 4, 5, 6, 7, 8, 9 or 10 amino acid substitutions or deletions as compared to SEQ ID NO: 101. (Embodiment 3) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 102 or the heavy chain variable region comprises a sequence having about 1, 2, 3, 4, 5, 6, 7, 8, 9 or 10 amino acid substitutions or deletions as compared to SEQ ID NO: 102. (Embodiment 4) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 103 or the heavy chain variable region comprises a sequence having about 1, 2, 3, 4, 5, 6, 7, 8, 9 or 10 amino acid substitutions or deletions as compared to SEQ ID NO: 103. (Embodiment 5) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 104 or the heavy chain variable region comprises a sequence having about 1, 2, 3, 4, 5, 6, 7, 8, 9 or 10 amino acid substitutions or deletions as compared to SEQ ID NO: 104. (Embodiment 6) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 105 or the heavy chain variable region comprises a sequence having about 1, 2, 3, 4, 5, 6, 7, 8, 9 or 10 amino acid substitutions or deletions as compared to SEQ ID NO: 105. (Embodiment 7) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 106 or the heavy chain variable region comprises a sequence having about 1, 2, 3, 4, 5, 6, 7, 8, 9 or 10 amino acid substitutions or deletions as compared to SEQ ID NO: 106. (Embodiment 8) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 107 or the heavy chain variable region comprises a sequence having about 1, 2, 3, 4, 5, 6, 7, 8, 9 or 10 amino acid substitutions or deletions as compared to SEQ ID NO: 107. (Embodiment 9) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108 or the heavy chain variable region comprises a sequence having about 1, 2, 3, 4, 5, 6, 7, 8, 9 or 10 amino acid substitutions or deletions as compared to SEQ ID NO: 108. (Embodiment 10) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 109 or the heavy chain variable region comprises a sequence having about 1, 2, 3, 4, 5, 6, 7, 8, 9 or 10 amino acid substitutions or deletions as compared to SEQ ID NO: 109. (Embodiment 11) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 110 or the heavy chain variable region comprises a sequence having about 1, 2, 3, 4, 5, 6, 7, 8, 9 or 10 amino acid substitutions or deletions as compared to SEQ ID NO: 110. (Embodiment 12) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 111 or the heavy chain variable region comprises a sequence having about 1, 2, 3, 4, 5, 6, 7, 8, 9 or 10 amino acid substitutions or deletions as compared to SEQ ID NO: 111. (Embodiment 13) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 112 or the heavy chain variable region comprises a sequence having about 1, 2, 3, 4, 5, 6, 7, 8, 9 or 10 amino acid substitutions or deletions as compared to SEQ ID NO: 112. (Embodiment 14) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 113 or the heavy chain variable region comprises a sequence having about 1, 2, 3, 4, 5, 6, 7, 8, 9 or 10 amino acid substitutions or deletions as compared to SEQ ID NO: 113. (Embodiment 15) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 114 or the heavy chain variable region comprises a sequence having about 1, 2, 3, 4, 5, 6, 7, 8, 9 or 10 amino acid substitutions or deletions as compared to SEQ ID NO: 114. (Embodiment 16) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 115 or the heavy chain variable region comprises a sequence having about 1, 2, 3, 4, 5, 6, 7, 8, 9 or 10 amino acid substitutions or deletions as compared to SEQ ID NO: 115. (Embodiment 17) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 116 or the heavy chain variable region comprises a sequence having about 1, 2, 3, 4, 5, 6, 7, 8, 9 or 10 amino acid substitutions or deletions as compared to SEQ ID NO: 116. (Embodiment 18) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 117 or the heavy chain variable region comprises a sequence having about 1, 2, 3, 4, 5, 6, 7, 8, 9 or 10 amino acid substitutions or deletions as compared to SEQ ID NO: 117. (Embodiment 19) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 118 or the heavy chain variable region comprises a sequence having about 1, 2, 3, 4, 5, 6, 7, 8, 9 or 10 amino acid substitutions or deletions as compared to SEQ ID NO: 118. (Embodiment 20) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 119 or the heavy chain variable region comprises a sequence having about 1, 2, 3, 4, 5, 6, 7, 8, 9 or 10 amino acid substitutions or deletions as compared to SEQ ID NO: 119. (Embodiment 21) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 120 or the heavy chain variable region comprises a sequence having about 1, 2, 3, 4, 5, 6, 7, 8, 9 or 10 amino acid substitutions or deletions as compared to SEQ ID NO: 120. (Embodiment 22) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 121 or the heavy chain variable region comprises a sequence having about 1, 2, 3, 4, 5, 6, 7, 8, 9 or 10 amino acid substitutions or deletions as compared to SEQ ID NO: 121. (Embodiment 23) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122 or the heavy chain variable region comprises a sequence having about 1, 2, 3, 4, 5, 6, 7, 8, 9 or 10 amino acid substitutions or deletions as compared to SEQ ID NO: 122. (Embodiment 24) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 123 or the heavy chain variable region comprises a sequence having about 1, 2, 3, 4, 5, 6, 7, 8, 9 or 10 amino acid substitutions or deletions as compared to SEQ ID NO: 123. (Embodiment 25) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 124 or the heavy chain variable region comprises a sequence having about 1, 2, 3, 4, 5, 6, 7, 8, 9 or 10 amino acid substitutions or deletions as compared to SEQ ID NO: 124. (Embodiment 26) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 125 or the heavy chain variable region comprises a sequence having about 1, 2, 3, 4, 5, 6, 7, 8, 9 or 10 amino acid substitutions or deletions as compared to SEQ ID NO: 125. (Embodiment 27) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 126 or the heavy chain variable region comprises a sequence having about 1, 2, 3, 4, 5, 6, 7, 8, 9 or 10 amino acid substitutions or deletions as compared to SEQ ID NO: 126. (Embodiment 28) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 127 or the heavy chain variable region comprises a sequence having about 1, 2, 3, 4, 5, 6, 7, 8, 9 or 10 amino acid substitutions or deletions as compared to SEQ ID NO: 127. (Embodiment 29) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 128 or the heavy chain variable region comprises a sequence having about 1, 2, 3, 4, 5, 6, 7, 8, 9 or 10 amino acid substitutions or deletions as compared to SEQ ID NO: 128. (Embodiment 30) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 129 or the heavy chain variable region comprises a sequence having about 1, 2, 3, 4, 5, 6, 7, 8, 9 or 10 amino acid substitutions or deletions as compared to SEQ ID NO: 129. (Embodiment 31) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 130 or the heavy chain variable region comprises a sequence having about 1, 2, 3, 4, 5, 6, 7, 8, 9 or 10 amino acid substitutions or deletions as compared to SEQ ID NO: 130. (Embodiment 32) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 131 or the heavy chain variable region comprises a sequence having about 1, 2, 3, 4, 5, 6, 7, 8, 9 or 10 amino acid substitutions or deletions as compared to SEQ ID NO: 131. (Embodiment 33) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 132 or the heavy chain variable region comprises a sequence having about 1, 2, 3, 4, 5, 6, 7, 8, 9 or 10 amino acid substitutions or deletions as compared to SEQ ID NO: 132. (Embodiment 34) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 133 or the heavy chain variable region comprises a sequence having about 1, 2, 3, 4, 5, 6, 7, 8, 9 or 10 amino acid substitutions or deletions as compared to SEQ ID NO: 133. (Embodiment 35) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 134 or the heavy chain variable region comprises a sequence having about 1, 2, 3, 4, 5, 6, 7, 8, 9 or 10 amino acid substitutions or deletions as compared to SEQ ID NO: 134. (Embodiment 36) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 135 or the heavy chain variable region comprises a sequence having about 1, 2, 3, 4, 5, 6, 7, 8, 9 or 10 amino acid substitutions or deletions as compared to SEQ ID NO: 135. (Embodiment 37) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 136 or the heavy chain variable region comprises a sequence having about 1, 2, 3, 4, 5, 6, 7, 8, 9 or 10 amino acid substitutions or deletions as compared to SEQ ID NO: 136. (Embodiment 38) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 137 or the heavy chain variable region comprises a sequence having about 1, 2, 3, 4, 5, 6, 7, 8, 9 or 10 amino acid substitutions or deletions as compared to SEQ ID NO: 137. (Embodiment 39) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 138 or the heavy chain variable region comprises a sequence having about 1, 2, 3, 4, 5, 6, 7, 8, 9 or 10 amino acid substitutions or deletions as compared to SEQ ID NO: 138. (Embodiment 40) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 139 or the heavy chain variable region comprises a sequence having about 1, 2, 3, 4, 5, 6, 7, 8, 9 or 10 amino acid substitutions or deletions as compared to SEQ ID NO: 139. (Embodiment 41) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 140 or the heavy chain variable region comprises a sequence having about 1, 2, 3, 4, 5, 6, 7, 8, 9 or 10 amino acid substitutions or deletions as compared to SEQ ID NO: 140. (Embodiment 42) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 141 or the heavy chain variable region comprises a sequence having about 1, 2, 3, 4, 5, 6, 7, 8, 9 or 10 amino acid substitutions or deletions as compared to SEQ ID NO: 141. (Embodiment 43) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 142 or the heavy chain variable region comprises a sequence having about 1, 2, 3, 4, 5, 6, 7, 8, 9 or 10 amino acid substitutions or deletions as compared to SEQ ID NO: 142. (Embodiment 44) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 143 or the heavy chain variable region comprises a sequence having about 1, 2, 3, 4, 5, 6, 7, 8, 9 or 10 amino acid substitutions or deletions as compared to SEQ ID NO: 143. (Embodiment 45) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 144 or the heavy chain variable region comprises a sequence having about 1, 2, 3, 4, 5, 6, 7, 8, 9 or 10 amino acid substitutions or deletions as compared to SEQ ID NO: 144. (Embodiment 46) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 145 or the heavy chain variable region comprises a sequence having about 1, 2, 3, 4, 5, 6, 7, 8, 9 or 10 amino acid substitutions or deletions as compared to SEQ ID NO: 145. (Embodiment 47) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 146 or the heavy chain variable region comprises a sequence having about 1, 2, 3, 4, 5, 6, 7, 8, 9 or 10 amino acid substitutions or deletions as compared to SEQ ID NO: 146. (Embodiment 48) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 147 or the heavy chain variable region comprises a sequence having about 1, 2, 3, 4, 5, 6, 7, 8, 9 or 10 amino acid substitutions or deletions as compared to SEQ ID NO: 147. (Embodiment 49) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 148 or the heavy chain variable region comprises a sequence having about 1, 2, 3, 4, 5, 6, 7, 8, 9 or 10 amino acid substitutions or deletions as compared to SEQ ID NO: 148. (Embodiment 50) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 149 or the heavy chain variable region comprises a sequence having about 1, 2, 3, 4, 5, 6, 7, 8, 9 or 10 amino acid substitutions or deletions as compared to SEQ ID NO: 149. (Embodiment 51) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 150 or the heavy chain variable region comprises a sequence having about 1, 2, 3, 4, 5, 6, 7, 8, 9 or 10 amino acid substitutions or deletions as compared to SEQ ID NO: 150. (Embodiment 52) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 151 or the heavy chain variable region comprises a sequence having about 1, 2, 3, 4, 5, 6, 7, 8, 9 or 10 amino acid substitutions or deletions as compared to SEQ ID NO: 151. (Embodiment 53) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 152 or the heavy chain variable region comprises a sequence having about 1, 2, 3, 4, 5, 6, 7, 8, 9 or 10 amino acid substitutions or deletions as compared to SEQ ID NO: 152. (Embodiment 54) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 153 or the heavy chain variable region comprises a sequence having about 1, 2, 3, 4, 5, 6, 7, 8, 9 or 10 amino acid substitutions or deletions as compared to SEQ ID NO: 153. (Embodiment 55) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 154 or the heavy chain variable region comprises a sequence having about 1, 2, 3, 4, 5, 6, 7, 8, 9 or 10 amino acid substitutions or deletions as compared to SEQ ID NO: 154. (Embodiment 56) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 155 or the heavy chain variable region comprises a sequence having about 1, 2, 3, 4, 5, 6, 7, 8, 9 or 10 amino acid substitutions or deletions as compared to SEQ ID NO: 155. (Embodiment 57) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 156 or the heavy chain variable region comprises a sequence having about 1, 2, 3, 4, 5, 6, 7, 8, 9 or 10 amino acid substitutions or deletions as compared to SEQ ID NO: 156. (Embodiment 58) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 157 or the heavy chain variable region comprises a sequence having about 1, 2, 3, 4, 5, 6, 7, 8, 9 or 10 amino acid substitutions or deletions as compared to SEQ ID NO: 157. (Embodiment 59) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 158 or the heavy chain variable region comprises a sequence having about 1, 2, 3, 4, 5, 6, 7, 8, 9 or 10 amino acid substitutions or deletions as compared to SEQ ID NO: 158. (Embodiment 60) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 159 or the heavy chain variable region comprises a sequence having about 1, 2, 3, 4, 5, 6, 7, 8, 9 or 10 amino acid substitutions or deletions as compared to SEQ ID NO: 159. (Embodiment 61) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 160 or the heavy chain variable region comprises a sequence having about 1, 2, 3, 4, 5, 6, 7, 8, 9 or 10 amino acid substitutions or deletions as compared to SEQ ID NO: 160. (Embodiment 62) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 161 or the heavy chain variable region comprises a sequence having about 1, 2, 3, 4, 5, 6, 7, 8, 9 or 10 amino acid substitutions or deletions as compared to SEQ ID NO: 161. (Embodiment 63) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 162 or the heavy chain variable region comprises a sequence having about 1, 2, 3, 4, 5, 6, 7, 8, 9 or 10 amino acid substitutions or deletions as compared to SEQ ID NO: 162. (Embodiment 64) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 163 or the heavy chain variable region comprises a sequence having about 1, 2, 3, 4, 5, 6, 7, 8, 9 or 10 amino acid substitutions or deletions as compared to SEQ ID NO: 163. (Embodiment 65) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 164 or the heavy chain variable region comprises a sequence having about 1, 2, 3, 4, 5, 6, 7, 8, 9 or 10 amino acid substitutions or deletions as compared to SEQ ID NO: 164. (Embodiment 66) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 165 or the heavy chain variable region comprises a sequence having about 1, 2, 3, 4, 5, 6, 7, 8, 9 or 10 amino acid substitutions or deletions as compared to SEQ ID NO: 165. (Embodiment 67) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 166 or the heavy chain variable region comprises a sequence having about 1, 2, 3, 4, 5, 6, 7, 8, 9 or 10 amino acid substitutions or deletions as compared to SEQ ID NO: 166. (Embodiment 68) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 167 or the heavy chain variable region comprises a sequence having about 1, 2, 3, 4, 5, 6, 7, 8, 9 or 10 amino acid substitutions or deletions as compared to SEQ ID NO: 167. (Embodiment 69) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 168 or the heavy chain variable region comprises a sequence having about 1, 2, 3, 4, 5, 6, 7, 8, 9 or 10 amino acid substitutions or deletions as compared to SEQ ID NO: 168. (Embodiment 70) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 169 or the heavy chain variable region comprises a sequence having about 1, 2, 3, 4, 5, 6, 7, 8, 9 or 10 amino acid substitutions or deletions as compared to SEQ ID NO: 169.

(Embodiment 71) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201 or the light chain variable region comprises a sequence having about 1, 2, 3, 4, 5, 6, 7, 8, 9 or 10 amino acid substitutions or deletions as compared to SEQ ID NO: 201. (Embodiment 72) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202 or the light chain variable region comprises a sequence having about 1, 2, 3, 4, 5, 6, 7, 8, 9 or 10 amino acid substitutions or deletions as compared to SEQ ID NO: 202. (Embodiment 73) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 203 or the light chain variable region comprises a sequence having about 1, 2, 3, 4, 5, 6, 7, 8, 9 or 10 amino acid substitutions or deletions as compared to SEQ ID NO: 203. (Embodiment 74) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204 or the light chain variable region comprises a sequence having about 1, 2, 3, 4, 5, 6, 7, 8, 9 or 10 amino acid substitutions or deletions as compared to SEQ ID NO: 204. (Embodiment 75) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205 or the light chain variable region comprises a sequence having about 1, 2, 3, 4, 5, 6, 7, 8, 9 or 10 amino acid substitutions or deletions as compared to SEQ ID NO: 205. (Embodiment 76) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 206 or the light chain variable region comprises a sequence having about 1, 2, 3, 4, 5, 6, 7, 8, 9 or 10 amino acid substitutions or deletions as compared to SEQ ID NO: 206. (Embodiment 77) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 207 or the light chain variable region comprises a sequence having about 1, 2, 3, 4, 5, 6, 7, 8, 9 or 10 amino acid substitutions or deletions as compared to SEQ ID NO: 207. (Embodiment 78) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 208 or the light chain variable region comprises a sequence having about 1, 2, 3, 4, 5, 6, 7, 8, 9 or 10 amino acid substitutions or deletions as compared to SEQ ID NO: 208. (Embodiment 79) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 209 or the light chain variable region comprises a sequence having about 1, 2, 3, 4, 5, 6, 7, 8, 9 or 10 amino acid substitutions or deletions as compared to SEQ ID NO: 209. (Embodiment 80) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 210 or the light chain variable region comprises a sequence having about 1, 2, 3, 4, 5, 6, 7, 8, 9 or 10 amino acid substitutions or deletions as compared to SEQ ID NO: 210. (Embodiment 81) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 211 or the light chain variable region comprises a sequence having about 1, 2, 3, 4, 5, 6, 7, 8, 9 or 10 amino acid substitutions or deletions as compared to SEQ ID NO: 211. (Embodiment 82) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 212 or the light chain variable region comprises a sequence having about 1, 2, 3, 4, 5, 6, 7, 8, 9 or 10 amino acid substitutions or deletions as compared to SEQ ID NO: 212. (Embodiment 83) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 213 or the light chain variable region comprises a sequence having about 1, 2, 3, 4, 5, 6, 7, 8, 9 or 10 amino acid substitutions or deletions as compared to SEQ ID NO: 213. (Embodiment 84) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 214 or the light chain variable region comprises a sequence having about 1, 2, 3, 4, 5, 6, 7, 8, 9 or 10 amino acid substitutions or deletions as compared to SEQ ID NO: 214. (Embodiment 85) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 215 or the light chain variable region comprises a sequence having about 1, 2, 3, 4, 5, 6, 7, 8, 9 or 10 amino acid substitutions or deletions as compared to SEQ ID NO: 215. (Embodiment 86) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 216 or the light chain variable region comprises a sequence having about 1, 2, 3, 4, 5, 6, 7, 8, 9 or 10 amino acid substitutions or deletions as compared to SEQ ID NO: 216. (Embodiment 87) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 217 or the light chain variable region comprises a sequence having about 1, 2, 3, 4, 5, 6, 7, 8, 9 or 10 amino acid substitutions or deletions as compared to SEQ ID NO: 217. (Embodiment 88) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 218 or the light chain variable region comprises a sequence having about 1, 2, 3, 4, 5, 6, 7, 8, 9 or 10 amino acid substitutions or deletions as compared to SEQ ID NO: 218. (Embodiment 89) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 219 or the light chain variable region comprises a sequence having about 1, 2, 3, 4, 5, 6, 7, 8, 9 or 10 amino acid substitutions or deletions as compared to SEQ ID NO: 219. (Embodiment 90) The anti-TL1A antibody of any one of embodiments 1-70, wherein the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 220 or the light chain variable region comprises a sequence having about 1, 2, 3, 4, 5, 6, 7, 8, 9 or 10 amino acid substitutions or deletions as compared to SEQ ID NO: 220.

(Embodiment 91) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 101, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 92) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 102, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 93) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 103, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 94) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 104, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 95) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 105, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201.

(Embodiment 96) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 103, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 97) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 106, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 98) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 107, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 99) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 100) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 109, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202.

(Embodiment 101) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 102) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 109, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 103) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 203. (Embodiment 104) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 105) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 107, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204.

(Embodiment 106) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 107, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 107) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 110, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 108) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 111, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 109) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 112, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 110) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 113, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204.

(Embodiment 111) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 114, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 112) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 115, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 113) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 116, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 114) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 117, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 115) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 118, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204.

(Embodiment 116) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 114, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 117) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 102, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 118) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 104, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 119) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 119, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 120) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 119, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201.

(Embodiment 121) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 101, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 122) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 105, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 123) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 120, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 204. (Embodiment 124) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 121, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 125) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202.

(Embodiment 126) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 207. (Embodiment 127) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 123, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 128) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 124, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 202. (Embodiment 129) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 125, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 130) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 116, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205.

(Embodiment 131) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 117, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 132) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 126, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 133) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 127, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 134) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 127, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 135) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 121, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201.

(Embodiment 136) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 137) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 138) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 206. (Embodiment 139) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 124, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 140) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 124, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201.

(Embodiment 141) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 128, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 142) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 128, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 206. (Embodiment 143) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 129, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 144) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 130, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 145) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 131, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205.

(Embodiment 146) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 132, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 147) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 133, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 148) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 134, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 149) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 135, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 205. (Embodiment 150) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 126, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 151) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 130, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 152) The anti-TL1A antibody of embodiment 1, wherein the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 132, and the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 201. (Embodiment 153) The anti-TL1A antibody of embodiment 1, comprising A500. (Embodiment 154) The anti-TL1A antibody of embodiment 1, comprising A501.

Exemplary anti-TL1A Constant Regions

In some embodiments, one or more amino acid modifications may be introduced into the Fragment crystallizable (Fc) region of a human or humanized antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions. In an exemplary embodiment, the Fc region comprises any one of SEQ ID NOS: 320-367. In some embodiments, the anti-TL1A antibody comprises a constant region comprising any one of SEQ ID NOS: 319, 368-381.

In some embodiments, antibodies of this disclosure have a reduced effector function as compared to a human IgG. Effector function refers to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of target cells in the presence of complement, where the complement action pathway is initiated by the binding of C1q to antibody bound with the target.

Some Fc regions have a natural lack of effector function, and some Fc regions can comprise mutations that reduce effector functions. For instance, IgG4 has low ADCC and CDC activities and IgG2 has low ADCC activity.

The disclosure provides antibodies comprising Fc regions characterized by exhibiting ADCC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease ADCC (such as human IgG1, SEQ ID NO: 320). The disclosure provides antibodies comprising Fc regions characterized by exhibiting CDC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease CDC (such as human IgG1, SEQ ID NO: 320). In certain embodiments, the antibodies of this disclosure have reduced effector function as compared with human IgG1. In certain embodiments, antibodies herein have no detectable ADCC activity. In certain embodiments, the reduction and/or abatement of ADCC activity may be attributed to the reduced affinity antibodies of the invention exhibit for Fc ligands and/or receptors. In certain embodiments, antibodies herein exhibit no detectable CDC activities. In some embodiments, the reduction and/or abatement of CDC activity may be attributed to the reduced affinity antibodies of the invention exhibit for Fc ligands and/or receptors. Measurement of effector function may be performed as described in Example 3.

In some embodiments, antibodies comprising Fc regions described herein exhibit decreased affinities to C1q relative to an unmodified antibody (e.g., human IgG1 having SEQ ID NO: 320). In some embodiments, antibodies herein exhibit affinities for C1q receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than an unmodified antibody. In some embodiments, antibodies herein exhibit affinities for C1q that are at least 90%, at least 80%, at least 70%, at least 60%, at least 50%, at least 40%, at least 30%, at least 20%, at least 10%, or at least 5% less than an unmodified antibody.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. Measurement of effector function may be performed as described in Example 3.

In some embodiments, antibodies are tested for binding to Fcγ receptors and complement C1q by ELISA. In some embodiments, antibodies are tested for the ability to activate primary human immune cells in vitro, for example, by assessing their ability to induce expression of activation markers.

In some embodiments, assessment of ADCC activity of an anti-TL1A antibody comprises adding the antibody to target cells in combination with immune effector cells, which may be activated by the antigen antibody complexes resulting in cytolysis of the target cell. Cytolysis may be detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38. Alternatively, or additionally, ADCC activity of the antibody of interest may be assessed in vivo, e.g., in an animal model such as that disclosed in Clynes et al., 1998, PNAS USA 95:652-656.

In some embodiments, an assessment of complement activation, a CDC assay, may be performed as described in Gazzano-Santoro et al., 1996, J. Immunol. Methods, 202:163.

Non-limiting examples of Fc mutations in IgG1 that may reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat. In certain embodiments, the antibodies of this disclosure have reduced effector function as compared with human IgG1.

In some embodiments, an antibody comprises an IgG1 Fc region comprising one or more of the following substitutions according to the Kabat numbering system: N297A, N297Q, N297D, D265A, S228P, L235A, L237A, L234A, E233P, L234V, C236 deletion, P238A, A327Q, P329A, P329G, L235E, P331S, L234F, 235G, 235Q, 235R, 235S, 236F, 236R, 237E, 237K, 237N, 237R, 238A, 238E, 238G, 238H, 238I, 238V, 238W, 238Y, 248A, 254D, 254E, 254G, 254H, 254I, 254N, 254P, 254Q, 254T, 254V, 255N, 256H, 256K, 256R, 256V, 264S, 265H, 265K, 265S, 265Y, 267G, 267H, 267I, 267K, 268K, 269N, 269Q, 270A, 270G, 270M, 270N, 271T, 272N, 279F, 279K, 279L, 292E, 292F, 292G, 292I, 293S, 301W, 304E, 311E, 311G, 311S, 316F, 327T, 328V, 329Y, 330R, 339E, 339L, 3431, 343V, 373A, 373G, 373S, 376E, 376W, 376Y, 380D, 382D, 382P, 385P, 424H, 424M, 424V, 434I, 438G, 439E, 439H, 439Q, 440A, 440D, 440E, 440F, 440M, 440T, 440V.

In some embodiments, an antibody comprises a Fc region selected from the representative sequences disclosed in Table 3, Table 13, and Table 9B. In some embodiments, an antibody comprises an IgG1 Fc region comprising E233P, according to the Kabat numbering system. In some embodiments, an antibody comprises an IgG4 Fc region comprising S228P and L235E. In some embodiments, an antibody comprises an IgG1 Fc region comprising L235E, according to the Kabat numbering system. In some embodiments, an antibody comprises an IgG1 Fc region comprising L234A and L235A, according to the Kabat numbering system. In some embodiments, an antibody comprises an IgG1 Fc region comprising L234A, L235A, and G237A, according to the Kabat numbering system. In some embodiments, an antibody comprises an IgG1 Fc region comprising L234A, L235A, P329G, according to the Kabat numbering system. In some embodiments, an antibody comprises an IgG1 Fc region comprising L234F, L235E, and P331S, according to the Kabat numbering system. In some embodiments, an antibody comprises an IgG1 Fc region comprising L234A, L235E, and G237A, according to the Kabat numbering system. In some embodiments, an antibody comprises an IgG1 Fc region comprising L234A, L235E, G237A, and P331S, according to the Kabat numbering system. In some embodiments, an antibody comprises an IgG1 Fc region comprising L234A, L235A, G237A, P238S, H268A, A330S, and P331S (IgG1σ), according to the Kabat numbering system. In some embodiments, an antibody comprises an IgG1 Fc region comprising L234A, L235A, and P329A, according to the Kabat numbering system. In some embodiments, an antibody comprises an IgG1 Fc region comprising G236R and L328R, according to the Kabat numbering system. In some embodiments, an antibody comprises an IgG1 Fc region comprising G237A, according to the Kabat numbering system. In some embodiments, an antibody comprises an IgG1 Fc region comprising F241A, according to the Kabat numbering system. In some embodiments, an antibody comprises an IgG1 Fc region comprising V264A, according to the Kabat numbering system. In some embodiments, an antibody comprises an IgG1 Fc region comprising D265A, according to the Kabat numbering system. In some embodiments, an antibody comprises an IgG1 Fc region comprising D265A and N297A, according to the Kabat numbering system. In some embodiments, an antibody comprises an IgG1 Fc region comprising D265A and N297G, according to the Kabat numbering system. In some embodiments, an antibody comprises an IgG1 Fc region comprising D270A, according to the Kabat numbering system. In some embodiments, an antibody comprises an IgG1 Fc region comprising N297A, according to the Kabat numbering system. In some embodiments, an antibody comprises an IgG1 Fc region comprising N297G, according to the Kabat numbering system. In some embodiments, an antibody comprises an IgG1 Fc region comprising N297D, according to the Kabat numbering system. In some embodiments, an antibody comprises an IgG1 Fc region comprising N297Q, according to the Kabat numbering system. In some embodiments, an antibody comprises an IgG1 Fc region comprising P329A, according to the Kabat numbering system. In some embodiments, an antibody comprises an IgG1 Fc region comprising P329G, according to the Kabat numbering system. In some embodiments, an antibody comprises an IgG1 Fc region comprising P329R, according to the Kabat numbering system. In some embodiments, an antibody comprises an IgG1 Fc region comprising A330L, according to the Kabat numbering system. In some embodiments, an antibody comprises an IgG1 Fc region comprising P331A, according to the Kabat numbering system. In some embodiments, an antibody comprises an IgG1 Fc region comprising P331S, according to the Kabat numbering system. In some embodiments, an antibody comprises an IgG2 Fc region. In some embodiments, an antibody comprises an IgG4 Fc region. In some embodiments, an antibody comprises an IgG4 Fc region comprising S228P, according to the Kabat numbering system. In some embodiments, an antibody comprises an IgG4 Fc region comprising S228P, F234A, and L235A, according to the Kabat numbering system. In some embodiments, an antibody comprises an IgG2-IgG4 cross-subclass (IgG2/G4) Fc region. In some embodiments, an antibody comprises an IgG2-IgG3 cross-subclass Fc region. In some embodiments, an antibody comprises an IgG2 Fc region comprising H268Q, V309L, A330S, and P331S, according to the Kabat numbering system. In some embodiments, an antibody comprises an IgG2 Fc region comprising V234A, G237A, P238S, H268A, V309L, A330S, and P331S, according to the Kabat numbering system. In some embodiments, an antibody comprises a Fc region comprising high mannose glycosylation.

In some embodiments, an antibody comprises an IgG4 Fc region comprising a S228P substitution, according to the Kabat numbering system. In some embodiments, an antibody comprises an IgG4 Fc region comprising an A330S substitution, according to the Kabat numbering system. In some embodiments, an antibody comprises an IgG4 Fc region comprising a P331S substitution, according to the Kabat numbering system.

In some embodiments, an antibody comprises an IgG2 Fc region comprising an A330S substitution, according to the Kabat numbering system. In some embodiments, an antibody comprises an IgG2 Fc region comprising an P331S substitution, according to the Kabat numbering system. In some embodiments, an antibody comprises an IgG2 Fc region comprising an 234A substitution, according to the Kabat numbering system. In some embodiments, an antibody comprises an IgG2 Fc region comprising an 237A substitution, according to the Kabat numbering system.

In certain embodiments, an anti-TL1A described herein comprises a Fc region as shown in Table 13.

TABLE 13
Exemplary Fc Mutations

| | Constant Region (SEQ ID NO) | | |
|---|---|---|---|
| Mutations | K_DL | R_EM | K_EM |
| Wild-type IgG1 | 320 | 321 | 322 |
| L235E | 323 | 324 | 325 |
| L234A, L235A | 326 | 327 | 328 |
| L234A, L235A, G237A | 329 | 330 | 331 |
| L234A, L235A, P329G | 332 | 333 | 334 |
| L234F, L235E, P331S | 335 | 336 | 337 |
| L234A, L235E, G237A | 338 | 339 | 340 |
| L234A, L235E, G237A, P331S | 341 | 342 | 343 |
| L234A, L235A, P329A | 344 | 345 | 346 |
| D265A | 347 | 348 | 349 |
| N297G | 350 | 351 | 352 |
| D265A, N297A | 353 | 354 | 355 |
| D265A, N297G | 356 | 357 | 358 |
| L235A, G237A | 359 | 360 | 361 |
| Wild-type IgG4 | 362 | | |

In certain embodiments, an anti-TL1A antibody described herein comprises a Fc region comprising a sequence from Table 9B. In certain embodiments, an anti-TL1A antibody described herein comprises a Fc region comprising any one of SEQ ID NOS: 320-367 or a sequence at least about 90%, 91%, 92%, 93%, 94%, 95%, 96%, 97%, 98%, or 99% identical to any one of SEQ ID NOS: 320-367.

In some embodiments, anti-TL1A described herein comprise a light chain constant region comprising SEQ ID NO: 319 or a sequence at least about 90%, 91%, 92%, 93%, 94%, 95%, 96%, 97%, 98%, or 99% identical to SEQ ID NO: 319.

### Additional Non-Limiting Example Anti-TL1A Antibody Embodiments

### CDR Embodiments

In one aspect, provided herein is a first embodiment of an anti-TL1A antibody. As used herein, an anti-TL1A antibody includes an anti-TL1A antigen binding fragment. Non-limiting additional embodiments include: (Embodiment 2) The anti-TL1A antibody of embodiment 1, comprising a heavy chain comprising a HCDR1, a HCDR2, and a HCDR3, and a light chain comprising a LCDR1, a LCDR2, and a LCDR3. (Embodiment 3) The anti-TL1A antibody of embodiment 1, comprising a HCDR1 comprising SEQ ID NO: 1. (Embodiment 4) The anti-TL1A antibody of embodiment 1 or embodiment 2, comprising a HCDR2 comprising SEQ ID NO: 2. (Embodiment 5) The anti-TL1A antibody of embodiment 1 or embodiment 2, comprising a HCDR2 comprising SEQ ID NO: 3. (Embodiment 6) The anti-TL1A antibody of embodiment 1 or embodiment 2, comprising a HCDR2 comprising SEQ ID NO: 4. (Embodiment 7) The anti-TL1A antibody of embodiment 1 or embodiment 2, comprising a HCDR2 comprising SEQ ID NO: 5. (Embodiment 8) The anti-TL1A antibody of any one of embodiments 1-6, comprising a HCDR3 comprising SEQ ID NO: 6. (Embodiment 9) The anti-TL1A antibody of any one of embodiments 1-6, comprising a HCDR3 comprising SEQ ID NO: 7. (Embodiment 10) The anti-TL1A antibody of any one of embodiments 1-6, comprising a HCDR3 comprising SEQ ID NO: 8. (Embodiment 11) The anti-TL1A antibody of any one of embodiments 1-6, comprising a HCDR3 comprising SEQ ID NO: 9. (Embodiment 12) The anti-TL1A antibody of any one of embodiments 1-10, comprising a LCDR1 comprising SEQ ID NO: 10. (Embodiment 13) The anti-TL1A antibody of any one of embodiments 1-11, comprising a LCDR2 comprising SEQ ID NO: 11. (Embodiment 14) The anti-TL1A antibody of any one of embodiments 1-12, comprising a LCDR3 comprising SEQ ID NO: 12. (Embodiment 15) The anti-TL1A antibody of any one of embodiments 1-12, comprising a LCDR3 comprising SEQ ID NO: 13. (Embodiment 16) The anti-TL1A antibody of any one of embodiments 1-12, comprising a LCDR3 comprising SEQ ID NO: 14 or 15. (Embodiment 17) the anti-TL1A antibody of embodiment 1, comprising the CDRs of antibody A, B, C, D, E, F, G, H, I, A2, B2, C2, D2, E2, F2, G2, H2, or 12 (Table 10). (Embodiment 18) The anti-TL1A antibody of embodiment 1, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15. (Embodiment 19) The anti-TL1A antibody of embodiment 1, comprising a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12

### Framework Embodiments

(Embodiment 20) The anti-TL1A antibody of any one of embodiments 1-19, comprising a heavy chain framework comprising IGHV1-46*02. (Embodiment 21) The anti-TL1A antibody of any one of embodiments 1-19, comprising a heavy chain framework comprising a variant of IGHV1-46*02 comprising between about 1 and about 20 amino acid substitutions from SEQ ID NO: 316. (Embodiment 22) The anti-TL1A antibody of any one of embodiments 1-19, comprising a heavy chain framework comprising a variant of IGHV1-46*02 comprising between about 1 and about 9 amino acid substitutions from SEQ ID NO: 316. (Embodiment 23) The anti-TL1A antibody of any one of embodiments 1-19, comprising a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. (Embodiment 24) The anti-TL1A antibody of any one of embodiments 21-23, wherein the heavy chain framework substitution comprises Q1E, as determined by Aho or Kabat numbering. (Embodiment 25) The anti-TL1A antibody of any one of embodiments 21-24, wherein the heavy chain framework substitution comprises R45K, as determined by Aho or Kabat numbering. (Embodiment 26) The anti-TL1A antibody of any one of embodiments 21-25, wherein the heavy chain framework substitution comprises A47R, as determined by Aho or Kabat numbering. (Embodiment 27) The anti-TL1A antibody of any one of embodiments 21-26, wherein the heavy chain framework substitution comprises M55I, as determined by Aho or Kabat numbering. (Embodiment 28) The anti-TL1A antibody of any one of embodiments 21-27, wherein the heavy chain framework substitution comprises V78A, as determined by Aho or Kabat numbering. (Embodiment 29) The anti-TL1A antibody of any one of embodiments 21-28, wherein the heavy chain framework substitution comprises M80I, as determined by Aho or Kabat numbering. (Embodiment 30) The anti-TL1A antibody of any one of embodiments 21-29, wherein the heavy chain framework substitution comprises R82T, as determined by Aho or Kabat numbering. (Embodiment 31) The anti-TL1A antibody of any one of embodiments 21-30, wherein the heavy chain framework substitution comprises V89A, as determined by Aho or Kabat numbering. (Embodiment 32) The anti-TL1A antibody of any one of embodiments 21-31, wherein the heavy chain framework substitution comprises M91L, as determined by Aho or Kabat numbering.

(Embodiment 33) The anti-TL1A antibody of any one of embodiments 1-19, comprising a heavy chain framework comprising SEQ ID NO: 301. (Embodiment 34) The anti-TL1A antibody of embodiment 33, wherein X1 is Q. (Embodiment 35) The anti-TL1A of embodiment 33, wherein X1=E. (Embodiment 36) The anti-TL1A of any one of embodiments 33-35, wherein X2=R. (Embodiment 37) The anti-TL1A of any one of embodiments 33-35, wherein X2=K. (Embodiment 38) The anti-TL1A of any one of embodiments 33-37, wherein X3=A. (Embodiment 39) The anti-TL1A of any one of embodiments 33-37, wherein X3=R. (Embodiment 40) The anti-TL1A of any one of embodiments 33-39, wherein X4=M. (Embodiment 41) The anti-TL1A of any one of embodiments 33-39, wherein X4=I. (Embodiment 42) The anti-TL1A of any one of embodiments 33-41, wherein X5=V. (Embodiment 43) The anti-TL1A of any one of embodiments 33-41, wherein X5=A. (Embodiment 44) The anti-TL1A of any one of embodiments 33-43, wherein X6=M. (Embodiment 45) The anti-TL1A of any one of embodiments 33-43, wherein X6=I. (Embodiment 46) The anti-TL1A of any one of embodiments 33-45, wherein X7=R. (Embodiment 47) The anti-TL1A of any one of embodiments 33-45, wherein X7=T. (Embodiment 48) The anti-TL1A of any one of embodiments 33-47, wherein X8=V. (Embodiment 49) The anti-TL1A of any one of embodiments 33-47, wherein X8=A. (Embodiment 50) The anti-TL1A of any one of embodiments 33-49, wherein X9=M. (Embodiment 51) The anti-TL1A of any one of embodiments 33-49, wherein X9=L.

(Embodiment 52) The anti-TL1A antibody of any one of embodiments 1-51, comprising a light chain framework comprising IGKV3-20*01. (Embodiment 53) The anti-TL1A antibody of any one of embodiments 1-51, comprising a light chain framework comprising a variant of IGKV3-20*01 comprising between about 1 and about 20 amino acid substitutions from SEQ ID NO: 317. (Embodiment 54) The anti-TL1A antibody of any one of embodiments 1-51, comprising a light chain framework comprising a variant of IGKV3-20*01 comprising about 1 amino acid substitution from SEQ ID NO: 317. (Embodiment 55) The anti-TL1A antibody of any one of embodiments 1-51, comprising a light chain framework comprising a variant of IGKV3-20*01 comprising about 2 amino acid substitutions from SEQ ID NO: 317. (Embodiment 56) The anti-TL1A antibody of any one of embodiments 1-51, comprising a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. (Embodiment 57) The anti-TL1A antibody of any one of embodiments 53-56, wherein the light chain framework substitution comprises Q1E, as determined by Aho or Kabat numbering. (Embodiment 58) The anti-TL1A antibody of any one of embodiments 53-57, wherein the light chain framework substitution comprises R45K, as determined by Aho or Kabat numbering.

(Embodiment 59) The anti-TL1A antibody of any one of embodiments 1-51, comprising a light chain comprising a light chain framework comprising SEQ ID NO: 303. (Embodiment 60) The anti-TL1A antibody of embodiment 59, wherein X10 is L. (Embodiment 61) The anti-TL1A antibody of embodiment 59, wherein X10 is P. (Embodiment 62) The anti-TL1A antibody of any one of embodiments 59-61, wherein X11 is L. (Embodiment 63) The anti-TL1A antibody of any one of embodiments 59-61, wherein X11 is W.

(Embodiment 64) The anti-TL1A antibody of any one of embodiments 1-19, comprising a heavy chain variable framework region comprising a modified human IGHV1-46*02 framework, and a light chain variable framework region comprising a human IGKV3-20 framework or a modified human IGKV3-20 framework, wherein the heavy chain variable framework region and the light chain variable framework region collectively comprise at least one amino acid modification(s) as compared to the human IGHV1-46*02 framework and the human IGKV3-20 framework. (Embodiment 65) The antibody of embodiment 64, wherein the at least one amino acid modification(s) is no more than about 13, 12, 11, 10, 9, or 8 amino acid modifications. (Embodiment 66) The antibody of embodiment 64 or embodiment 65, wherein the amino acid modification(s) comprise: a modification at amino acid position 45 in the heavy chain variable region. (Embodiment 67) The antibody of any one of embodiments 64-66, wherein the amino acid modification(s) comprise a modification at amino acid position 47 in the heavy chain variable region. (Embodiment 68) The antibody of any one of embodiments 64-67, wherein the amino acid modification(s) comprise a modification at amino acid position 55 in the heavy chain variable region. (Embodiment 69) The antibody of any one of embodiments 64-68, wherein the amino acid modification(s) comprise a modification at amino acid position 78 in the heavy chain variable region. (Embodiment 70) The antibody of any one of embodiments 64-69, wherein the amino acid modification(s) comprise a modification at amino acid position 80 in the heavy chain variable region. (Embodiment 71) The antibody of any one of embodiments 64-70, wherein the amino acid modification(s) comprise a modification at amino acid position 82 in the heavy chain variable region. (Embodiment 72) The antibody of any one of embodiments 64-71, wherein the amino acid modification(s) comprise a modification at amino acid position 89 in the heavy chain variable region. (Embodiment 73) The antibody of any one of embodiments 64-72, wherein the amino acid modification(s) comprise a modification at amino acid position 91 in the heavy chain variable region, per Aho or Kabat numbering. (Embodiment 74) The antibody of any one of embodiments 64-65, wherein the amino acid modification(s) comprise (a) R45K, (b) A47R, (c) M55I, (d) V78A, (e) M80I, (f) R82T, (g) V89A, or (h) M91L in the heavy chain variable region, per Aho or Kabat numbering; or a combination of two or more modifications selected from (a) to (h). (Embodiment 75) The antibody of embodiment 74, wherein the amino acid modification(s) comprise: A47R. (Embodiment 76) The antibody of embodiment 74, wherein the amino acid modification(s) comprise: A47R, M55I, V78A, M80I, R82T, V89A, and M91L; A47R, M80I, and R82T; A47R, M80I, R82T, V89A, and M91L; or A47R, M55I, V78A, M80I, V89A, and M91L. (Embodiment 77) The antibody of embodiment 74, wherein the amino acid modification(s) comprise: R45K and A47R. (Embodiment 78) The antibody of embodiment 74, wherein the amino acid modification(s) comprise: R45K, A47R, V89A, and M91L. (Embodiment 79) The antibody of embodiment 74, wherein the amino acid modification(s) comprise: R45K and A47R, and M80I. (Embodiment 80) The antibody of embodiment 74, wherein the amino acid modification(s) comprise: R45K, A47R, M80I, and M91L; R45K, A47R, V78A, M80I, V89A, and M91L; R45K, A47R, M55I, V78A, M80I, R82T, V89A, and M91L; R45K, A47R, M80I, V89A, and M91L; R45K, A47R, M55I, M80I, R82T, V89A, and M91L; R45K, A47R, M80I, and V89A; R45K, A47R, M80I, R82T, V89A, M91L; or R45K, A47R, M55I, M80I, V89A, and M91L. (Embodiment 81) The antibody of embodiment 74, wherein the amino acid modification(s) comprise: R45K. (Embodiment 82) The antibody of embodiment 74, wherein the amino acid modification(s) comprise: R45K and V78A. (Embodiment 83) The antibody of embodiment 74, wherein the amino acid modification(s) comprise: V78A. (Embodiment 84) The antibody of embodiment 74, wherein the amino acid modification(s) comprise: V78A and V89A; V78A and M80I; or V78A, M80I, and R82T. (Embodiment 85) The antibody of embodiment 74, wherein the amino acid modification(s) comprise: V89A. (Embodiment 86) The antibody of embodiment 74, wherein the amino acid modification(s) comprise: M80I. (Embodiment 87) The antibody of any one of embodiments 64-86, wherein the amino acid modification(s) comprises: (a) a modification at amino acid position 54 in the light chain variable region; and/or (b) a modification at amino acid position 55 in the light chain variable region, per Aho or Kabat numbering. (Embodiment 88) The antibody of embodiment 87, wherein the amino acid modification(s) comprises L54P in the light chain variable region, per Aho or Kabat numbering. (Embodiment 89) The antibody of embodiment 87 or 88, wherein the amino acid modification(s) comprises L55W in the light chain variable region, per Aho or Kabat numbering.

(Embodiment 90) The antibody of any one of embodiments 1-19, comprising a heavy chain FR1 as set forth by SEQ ID NO: 304. (Embodiment 91) The antibody of any one of embodiments 1-19 or 90, comprising a heavy chain FR2 as set forth by SEQ ID NO: 305. (Embodiment 92) The antibody of any one of embodiments 1-19 or 90, comprising a heavy chain FR2 as set forth by SEQ ID NO: 313. (Embodiment 93) The antibody of any one of embodiments 1-19 or 90-92, comprising a heavy chain FR3 as set forth by SEQ ID NO: 306. (Embodiment 94) The antibody of any one of embodiments 1-19 or 90-92, comprising a heavy chain FR3 as set forth by SEQ ID NO: 307. (Embodiment 95) The antibody of any one of embodiments 1-19 or 90-92, comprising a heavy chain FR3 as set forth by SEQ ID NO: 314. (Embodiment 96) The antibody of any one of embodiments 1-19 or 90-92, comprising a heavy chain FR3 as set forth by SEQ ID NO: 315. (Embodiment 97) The antibody of any one of embodiments 1-19 or 90-96, comprising a heavy chain FR4 as set forth by SEQ ID NO: 308. (Embodiment 98) The antibody of any one of embodiments 1-19 or 90-97, comprising a light chain FR1 as set forth by SEQ ID NO: 309. (Embodiment 99) The antibody of any one of embodiments 1-19 or 90-98, comprising a light chain FR2 as set forth by SEQ ID NO: 310. (Embodiment 100) The antibody of any one of embodiments 1-19 or 90-99, comprising a light chain FR3 as set forth by SEQ ID NO: 311. (Embodiment 101) The antibody of any one of embodiments 1-19 or 90-100, comprising a light chain FR4 as set forth by SEQ ID NO: 312. (Embodiment 102) The antibody of any one of embodiments 1-19, comprising a HC FR1 as set forth by SEQ ID NO: 304, a HC FR2 as set forth by SEQ ID NO: 305, a HC FR3 as set forth by SEQ ID NO: 307, a HC FR4 as set forth by SEQ ID NO: 308, a LC FR1 as set forth by SEQ ID NO: 309, a LC FR2 as set forth by SEQ ID NO: 310, a LC FR3 as set forth by SEQ ID NO: 311, and a LC FR4 as set forth by SEQ ID NO: 312.

### Variable Region Embodiments

(Embodiment 103) The antibody of embodiment 1, comprising a heavy chain variable domain comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 101-169, and a light chain variable domain comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 201-220. (Embodiment 104) The antibody of embodiment 103, comprising a heavy chain variable domain comprising an amino acid sequence at least 96% identical to SEQ ID NO: 104, and a light chain variable domain comprising an amino acid sequence at least 97% identical to SEQ ID NO: 201. (Embodiment 105) The antibody of embodiment 103, comprising an amino acid sequence at least 97% identical to SEQ ID NO: 104. (Embodiment 106) The antibody of embodiment 103, comprising an amino acid sequence at least 98% identical to SEQ ID NO: 104. (Embodiment 107) The antibody of embodiment 103, comprising an amino acid sequence at least 99% identical to SEQ ID NO: 104. (Embodiment 108) The antibody of embodiment 103, comprising SEQ ID NO: 104. (Embodiment 109) The antibody of any one of embodiments 103-108, comprising an amino acid sequence at least 98% identical to SEQ ID NO: 201. (Embodiment 110) The antibody of embodiment 109, comprising an amino acid sequence at least about 99% identical to SEQ ID NO: 201. (Embodiment 111) The antibody of embodiment 109, comprising SEQ ID NO: 201.

(Embodiment 112) The antibody of embodiment 103, comprising a heavy chain variable domain comprising an amino acid sequence at least about 97% identical to SEQ ID NO: 104, and a light chain variable domain comprising an amino acid sequence at least about 97% identical to SEQ ID NO: 201. (Embodiment 113) The antibody of embodiment 112, wherein the heavy chain variable domain comprises an amino acid sequence at least about 98% identical to SEQ ID NO: 104. (Embodiment 114) The antibody of embodiment 112, wherein the heavy chain variable domain comprises an amino acid sequence at least about 99% identical to SEQ ID NO: 104. (Embodiment 115) The antibody of embodiment 112, wherein the heavy chain variable domain comprises SEQ ID NO: 104. (Embodiment 116) The antibody of any one of embodiments 112-115, wherein the light chain variable domain comprises an amino acid sequence at least about 98% identical to SEQ ID NO: 201. (Embodiment 117) The antibody of any one of embodiments 112-116, wherein the light chain variable domain comprises an amino acid sequence at least about 99% identical to SEQ ID NO: 201. (Embodiment 118) The antibody of any one of embodiments 112-117, wherein the light chain variable domain comprises SEQ ID NO: 201.

### Fc Region Embodiments

(Embodiment 119) The antibody of any one of embodiments 1-118, comprising a fragment crystallizable (Fc) region. (Embodiment 120) The antibody of embodiment 119, comprising reduced antibody-dependent cell-mediated cytotoxicity (ADCC) function as compared to human IgG1 and/or reduced complement-dependent cytotoxicity (CDC) as compared to human IgG1. (Embodiment 121) The antibody of embodiment 120, wherein the human IgG1 comprises SEQ ID NO: 320. (Embodiment 122) The antibody of embodiment 120 or embodiment 121, wherein the ADCC function of the Fc region comprising reduced ADCC is at least about 50% reduced as compared to human IgG1. (Embodiment 123) The antibody of any one of embodiments 120-122, wherein the CDC function of the Fc region comprising reduced ADCC is at least about 50% reduced as compared to human IgG1. (Embodiment 124) The anti-TL1A antibody of any one of embodiments 119-123, comprising a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238W, or 238Y, (n) 248A, (0) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(uu), per Kabat numbering. (Embodiment 125) The anti-TL1A of any one of embodiments 119-123, comprising a (i) human IgG4 Fc region or (ii) a human IgG4 Fc region comprising (a) S228P, (b) S228P and L235E, or (c) S228P, F234A, and L235A, per Kabat numbering. (Embodiment 126) The anti-TL1A of any one of embodiments 119-123, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331S (IgG20). (Embodiment 127) The antibody of any one of embodiments 119-123, comprising a human IgG1 comprising one or more substitutions selected from the group comprising 329A, 329G, 329Y, 331S, 236F, 236R, 238A, 238E, 238G, 238H, 238I, 238V, 238W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering. (Embodiment 128) The anti-TL1A of any one of embodiments 119-123, comprising a heavy chain Fc region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 320-362. (Embodiment 129) The anti-TL1A of any one of embodiments 119-123, comprising a heavy chain Fc region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 368-380. (Embodiment 130) The anti-TL1A of any one of embodiments 119-123, comprising a constant region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 381.

### Additional Antibody Features

(Embodiment 131) The anti-TL1A antibody of any one of embodiments 1-130, comprising a light chain constant region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 319.

(Embodiment 132) The anti-TL1A antibody of any one of embodiments 1-131, comprising at least about 80%, 81%, 82%, 83%, 84%, 85%, 86%, 87%, 88%, 89%, 90%, 91%, 92%, 93%, 94%, 95%, 96%, 97%, 98%, or 99% monomeric fraction as determined by size exclusion chromatography. (Embodiment 133), The antibody of embodiment 132, wherein the size exclusion chromatography comprises injecting purified antibody onto a size exclusion column, wherein the antibody is purified by protein A. (Embodiment 134) The antibody of embodiment 132 or 133, wherein the antibody is purified as described in Example 2. (Embodiment 135) The antibody of any one of embodiments 132-134, wherein the antibody is expressed under conditions described in Example 2. (Embodiment 136) The antibody of any one of embodiments 132-135, wherein the size exclusion chromatography column has an inner diameter of 4.6 mm. (Embodiment 137) The antibody of any one of embodiments 132-136, wherein the size exclusion chromatography column has a length of 150 mm. (Embodiment 138) The antibody of any one of embodiments 132-137, wherein the size exclusion chromatography column has a pore size of 200 Å. (Embodiment 139) The antibody of any one of embodiments 132-138, wherein the size exclusion chromatography column has a particle size of 1.7 micrometer. (Embodiment 140) The antibody of any one of embodiments 132-139, wherein the size exclusion chromatography column is ACQUITY UPLC BEH200 SEC column. (Embodiment 141) The antibody of any one of embodiments 132-140, wherein the antibody or antigen binding fragment is injected at a total volume of 15 μL. (Embodiment 142) The antibody of any one of embodiments 132-141, wherein the antibody is injected at a concentration of about 0.1 μg/μL to about 1.0 μg/μL. (Embodiment 143) The antibody of any one of embodiments 132-142, wherein the size exclusion chromatography is performed on a Shimadzu UPLC instrument. (Embodiment 144) The antibody of any one of embodiments 132-143, wherein the size exclusion chromatography is performed at a flow rate of 0.2 mL/min. (Embodiment 145) The antibody of any one of embodiments 132-144, wherein the size exclusion chromatography is performed at a column oven temperature of 30° C. (Embodiment 146) The antibody of any one of embodiments 132-145, wherein the percentage of monomer is calculated using Shimadzu software. (Embodiment 147) The antibody of any one of embodiments 132-146, wherein the size exclusion chromatography is performed as described in Example 2.

(Embodiment 148) The anti-TL1A antibody of any one of embodiments 1-147, wherein the anti-TL1A is expressed at a concentration of at least about 2 μg/mL, between about 2 μg/mL and about 60 μg/mL, between about 5 μg/mL and about 60 μg/mL, between about 10 μg/mL and about 60 μg/mL, at least about 5 μg/mL, at least about 10 μg/mL, at least about 15 μg/mL, at least about 20 μg/mL, between about 2 μg/mL and about 50 μg/mL, between about 2 μg/mL and about 40 μg/mL, between about 2 μg/mL and about 30 μg/mL, between about 2 μg/mL and about 20 μg/mL, between about 5 μg/mL and about 50 μg/mL, between about 5 μg/mL and about 40 μg/mL, between about 5 μg/mL and about 30 μg/mL, between about 10 μg/mL and about 50 μg/mL, between about 10 μg/mL and about 40 μg/mL, or between about 10 μg/mL and about 30 μg/mL, as determined by a method disclosed herein. (Embodiment 149) The anti-TL1A antibody of any one of embodiments 1-147, wherein the expression level is at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 μg/mL as determined by a method disclosed herein. (Embodiment 150) The antibody of embodiment 148 or embodiment 149, wherein the antibody is expressed in FreeStyle 293-F cells. (Embodiment 151) The antibody of any one of embodiments 148-150, wherein the antibody is expressed as described in Example 2. (Embodiment 152) The antibody of any one of embodiments 148-151, wherein the antibody expression level is quantified using Enzyme-Linked Immunosorbent assay (ELISA). (Embodiment 153) The antibody of embodiment 152, wherein the ELISA comprises coating a surface of a substrate with a capture antibody that binds to a human or humanized antibody, applying the anti-TL1A antibody to the substrate, and applying to the substrate a second antibody that binds to a human or humanized antibody. (Embodiment 154) The antibody of embodiment 153, where the capture antibody comprises an anti-kappa antibody. (Embodiment 155) The antibody of embodiment 153 or embodiment 154, where the second antibody comprises an anti-Fc antibody. (Embodiment 156) The antibody of any one of embodiments 152-155, where the ELISA is performed as described in Example 2.

(Embodiment 157) A method of treating inflammatory bowel disease (IBD) in a subject in need thereof, the method comprising administering to the subject an antibody or antigen binding fragment of any one of embodiments 1-156. (Embodiment 158) The method of embodiment 157, wherein the IBD comprises Crohn's Disease. (Embodiment 159) The method of embodiment 157, wherein the IBD comprises ulcerative colitis.

(Embodiment 160) A nucleic acid encoding the antibody of any one of embodiments 1-156. (Embodiment 161) A vector comprising the nucleic acid of embodiment 160. (Embodiment 162) A cell comprising the nucleic acid of embodiment 160. (Embodiment 163) A cell comprising the vector of embodiment 161.

### Antibody Properties

Anti-TL1A antibodies described herein bind to specific regions or epitopes of human TL1A. In various embodiments, an anti-TL1A antibody provided herein has a binding affinity to human TL1A of less than about 1E⁻⁷, 1E⁻⁸, 1E⁻⁹, or 1E⁻¹⁰ Kd. In some cases, the binding affinity is from about 1E⁻⁹ to about 1E⁻¹⁰ Kd. In some embodiments, an anti-TL1A antibody provided herein has a binding affinity to murine TL1A and/or rat TL1A of less than about 1E⁻⁷, 1E⁻⁸, 1E⁻⁹, 1E⁻¹⁰, or 1E⁻¹¹ Kd. Methods for determining binding affinity are exemplified herein, including in Example 2.

In various embodiments, an anti-TL1A antibody provided herein is an antagonist of a TL1A receptor, such as, but not limited to, DR3 and TR6/DcR3. In certain embodiments, the antibody inhibits at least about 10%, at least about 20%, at least about 30%, at least about 50%, at least about 75%, at least about 90%, or about 100% of one or more activity of the bound TL1A receptor. In certain embodiments, the anti-TL1A antibody inhibits TL1A activation as measured by interferon gamma release in human blood. In certain embodiments, the antibody inhibits interferon gamma release in human blood at an IC₅₀ of between about 1 nanomolar and about 30 picomolar. In certain embodiments, the antibody inhibits interferon gamma release in human blood at an IC₅₀ of between about 500 picomolar and about 30 picomolar. In certain embodiments, the antibody inhibits interferon gamma release in human blood at an IC₅₀ of between about 200 picomolar and about 30 picomolar. In certain embodiments, the antibody inhibits interferon gamma release in human blood at an IC₅₀ of less than or equal to about 200 picomolar. In certain embodiments, the antibody inhibits interferon gamma release in human blood at an IC₅₀ of less than or equal to about 100 picomolar.

In various embodiments, an anti-TL1A antibody provided herein comprises at least about 80% monomeric fraction after expression and purification as described in Example 2 or elsewhere herein. In various embodiments, an anti-TL1A antibody provided herein comprises at least about 85% monomeric fraction after expression and purification as described in Example 2 or elsewhere herein. In various embodiments, an anti-TL1A antibody provided herein comprises at least about 90% monomeric fraction after expression and purification as described in Example 2 or elsewhere herein. In various embodiments, an anti-TL1A antibody provided herein comprises at least about 91%, 92%, 93%, 94%, 95%, 96%, 97%, 98%, 99%, or 100% monomeric fraction after expression and purification as described in Example 2 or elsewhere herein.

In various embodiments, an anti-TL1A antibody provided herein has at least about 2 μg/mL expression as determined by the method disclosed herein. In some embodiments, the anti-TL1A antibody has about 2 μg/mL to about 60 μg/mL expression as determined by the method disclosed herein. In some embodiments, the anti-TL1A antibody has about 5 μg/mL to about 60 μg/mL expression as determined by the method disclosed herein. In some embodiments, the anti-TL1A antibody has about 10 μg/mL to about 60 μg/mL expression as determined by the method disclosed herein. In some embodiments, the anti-TL1A antibody has at least about 5 μg/mL expression as determined by the method disclosed herein. In some embodiments, the anti-TL1A antibody has at least about 10 μg/mL expression as determined by the method disclosed herein. In some embodiments, the anti-TL1A antibody has at least about 15 μg/mL expression as determined by the method disclosed herein. In some embodiments, the anti-TL1A antibody has at least about 20 μg/mL expression as determined by the method disclosed herein. In some embodiments, the anti-TL1A antibody expresses between about 2 μg/mL and about 50 μg/mL, between about 2 μg/mL and about 40 μg/mL, between about 2 μg/mL and about 30 μg/mL expression, between about 2 μg/mL and about 20 μg/mL, between about 5 μg/mL and about 50 μg/mL, between about 5 μg/mL and about 40 μg/mL, between about 5 μg/mL and about 30 μg/mL, between about 10 μg/mL and about 50 μg/mL, between about 10 μg/mL and about 40 μg/mL, or between about 10 μg/mL and about 30 μg/mL as determined by the method disclosed herein. In some embodiments, the anti-TL1A antibody has about 2, 3, 4, 5, 6, 7, 8, 9, 10, 11, 12, 13, 14, 15, 16, 17, 18, 19, 20, 21, 22, 23, 24, 25, 26, 27, 28, 29, 30 μg/mL expression as determined by the method disclosed herein. Methods disclosed herein include those described in Example 2.

In various embodiments, an anti-TL1A antibody provided herein is humanized and has less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. The humanized heavy chain variable domain may comprise IGHV1-46*02 framework with no or fewer than about 10, 9, 8, 7, 6, 5, 4, 3, 2, or 1 non-human mutations. The humanized light chain variable domain may comprise IGKV3-20 framework with no or fewer than about 10, 9, 8, 7, 6, 5, 4, 3, 2, or 1 non-human mutations.

### Epitope

Various embodiments provide for an anti-TL1A antibody that binds to the same region of a TL1A protein or portion thereof as a reference antibody such as the anti-TL1A antibodies described herein. In some embodiments, the reference antibody comprises antibody A, B, C, D, E, F, G, H, A2, B2, C2, D2, E2, F2, G2, or H2, or a combination thereof. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 104, and a light chain comprising a sequence at least about 90%, 92%, 93%, 94%, 95%, 96%, 97%, 98%, 99% or 100% identical to SEQ ID NO: 201. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 107, and a light chain comprising a sequence at least about 90%, 92%, 93%, 94%, 95%, 96%, 97%, 98%, 99% or 100% identical to SEQ ID NO: 201.

Non-limiting methods for determining whether an anti-TL1A antibody (i.e. test antibody) binds to the same region of a TL1A protein or portion thereof as an antibody described herein are provided. An exemplary embodiment comprises a competition assay. For instance, the method comprises determining whether the test antibody can compete with binding between the reference antibody and the TL1A protein or portion thereof, or determining whether the reference antibody can compete with binding between the test antibody and the TL1A protein or portion thereof. Exemplary methods include use of surface plasmon resonance to evaluate whether an anti-TL1A antibody can compete with the binding between TL1A and another anti-TL1A antibody. In some cases, surface plasmon resonance is utilized in the competition assay. Non-limiting methods are described in the examples.

In certain embodiments, disclosed herein are antibodies that compete for binding TL1A with the antibodies described herein. In certain embodiments, disclosed herein are antibodies that bind a discrete epitope that overlaps with an epitope of TL1A bound by an antibody described herein. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 104; and a light chain variable region comprising the amino acid of SEQ ID NO: 201. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 107; and a light chain variable region comprising the amino acid of SEQ ID NO: 201.

### Other Anti-TL1A Antibodies

Other anti-TL1A antibodies with validated efficacy against inflammatory disease or conditions are also provided for the combination therapy. In some embodiments, the anti-TL1A antibody antigen-binding fragment thereof specifically binds TL1A and comprises: (a) a heavy chain variable region (VH) comprising a CDR-H1 comprising the amino acid sequence of GYX1FX2X3YGIS (wherein X1 is P, S, D, Q, N, X2 is T, or R, X3 is N, T, Y, or H, SEQ ID NO: 401), a CDR-H2 comprising the amino acid sequence of WISX1YNGX2X3X4YAX5X6X7QG (wherein X1 is T, P, S, or A, X2 is N, G, V, K, or A, X3 is T or K, X4 is H or N, X5 is Q or R, X6 is K or M, X7 is L or H, SEQ ID NO: 402), and a CDR-H3 comprising the amino acid sequence of ENYYGSGX1X2RGGMDX3 (wherein X1 is S or A, X2 is Y or F, X3 is V, A, or G, SEQ ID NO: 403); and (b) a light chain variable region (VL) comprising a CDR-L1 comprising the amino acid sequence of RASQSVSSYLA (SEQ ID NO: 404), a CDR-L2 comprising the amino acid sequence of DASNRAT (SEQ ID NO: 405), and a CDR-L3 comprising the amino acid sequence of QQRSNWPWT (SEQ ID NO: 406).

In one embodiment, the anti-TL1A antibody antigen-binding fragment thereof specifically binds TL1A and comprises: (a) a heavy chain variable region (VH) comprising a CDR-H1 comprising the amino acid sequence of GYDFTYYGIS (SEQ ID NO: 407), a CDR-H2 comprising the amino acid sequence of WISTYNGNTHYARMLQG (SEQ ID NO: 408), and a CDR-H3 comprising the amino acid sequence of ENYYGSGAYRGGMDV (SEQ ID NO: 409); and (b) a light chain variable region (VL) comprising a CDR-L1 comprising the amino acid sequence of RASQSVSSYLA (SEQ ID NO: 404), a CDR-L2 comprising the amino acid sequence of DASNRAT (SEQ ID NO: 405), and a CDR-L3 comprising the amino acid sequence of QQRSNWPWT (SEQ ID NO: 406).

In another embodiment, the anti-TL1A antibody antigen-binding fragment thereof comprises: (a) a VH comprising the amino acid sequence of QVQLVQSGAEVKKPGASVKVSCKASGYDFTYYGISWVRQAPGQGLEWMGWISTYN GNTHYARMLQGRVTMTTDTSTRTAYMELRSLRSDDTAVYYCARENYYGSGAYRG GMDVWGQGTTVTVSS (SEQ ID NO: 410); and (b) a VL comprising the amino acid sequence of EIVLTQSPATLSLSPGERATLSCRASQSVSSYLAWYQQKPGQAPRLLIYDASNRATGI PARFSGSGSGTDFTLTISSLEPEDFAVYYCQQRSNWPWTFGQGTKVEIK (SEQ ID NO: 411).

In another embodiment, the anti-TL1A antibody antigen-binding fragment thereof comprises: (a) a heavy chain comprising the amino acid sequence of QVQLVQSGAEVKKPGASVKVSCKASGYDFTYYGISWVRQAPGQGLEWMGWISTYN GNTHYARMLQGRVTMTTDTSTRTAYMELRSLRSDDTAVYYCARENYYGSGAYRG GMDVWGQGTTVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDKKV EPKSCDKTHTCPPCPAPEAAGAPSVFLFPPKPKDTLMISRTPEVTCVVVDVSHEDPEV KFNWYVDGVEVHNAKTKPREEQYNSTYRVVSVLTVLHQDWLNGKEYKCKVSNKA LPAPIEKTISKAKGQPREPQVYTLPPSREEMTKNQVSLTCLVKGFYPSDIAVEWESNG QPENNYKTTPPVLDSDGSFFLYSKLTVDKSRWQQGNVFSCSVMHEALHNHYTQKSL SLSPG (SEQ ID NO: 412); and (b) a light chain comprising the amino acid sequence of EIVLTQSPATLSLSPGERATLSCRASQSVSSYLAWYQQKPGQAPRLLIYDASNRATGI PARFSGSGSGTDFTLTISSLEPEDFAVYYCQQRSNWPWTFGQGTKVEIKRTVAAPSVF IFPPSDEQLKSGTASVVCLLNNFYPREAKVQWKVDNALQSGNSQESVTEQDSKDSTY SLSSTLTLSKADYEKHKVYACEVTHQGLSSPVTKSFNRGEC (SEQ ID NO: 413).

The disclosure further provides anti-TL1A antibodies or antigen binding fragments described in U.S. Pat. No. 9,683,998, which is hereby incorporated in its entirety by reference.

The efficacy of the anti-TL1A antibodies in the preceding 5 paragraphs have been validated in animal and clinical studies as further described in U.S. Pat. No. 9,683,998; Banfield C, et al. Br J Clin Pharmacol. 2020; 86:812-824; Danese S, et al. Clin Gastroenterol Hepatol. 2021 Jun. 11; S1542-3565 (21) 00614-5; Danese S, et al. Clin Gastroenterol Hepatol. 2021 November; 19 (11): 2324-2332.e6; Hassan-Zahraee M, et al. Inflammatory Bowel Diseases 2021, XX, 1-13; the disclosures of all of which, including the anti-TL1A antibodies tested therein, the study design and study results, are incorporated hereby in their entireties by reference.

In certain embodiments, the anti-TL1A antibody antigen-binding fragment thereof comprises: a heavy chain variable region CDR1 comprising the amino acid sequence of GYTFTSYDIN (SEQ ID NO: 414), a heavy chain variable region CDR2 comprising the amino acid sequence of WLNPNSGYTG (SEQ ID NO: 415), a heavy chain variable region CDR3 comprising the amino acid sequence of EVPETAAFEY (SEQ ID NO: 416), a light chain variable region CDR1 comprising the amino acid sequence of TSSSSDIGAGLGVH (SEQ ID NO: 417), a light chain variable region CDR2 comprising the amino acid sequence of GYYNRPS (SEQ ID NO: 418), and a light chain variable region CDR3 comprising the amino acid sequence of QSWDGTLSAL (SEQ ID NO: 419), wherein the antibody specifically binds to TNF-like ligand 1A (TL1A).

In one embodiment, the anti-TL1A antibody antigen-binding fragment thereof comprises: (a) a VH comprising the amino acid sequence of QVQLVQSGAEVKKPGASVKVSCKASGYTFTSYDINWVRQAPGQGLEWMGWLNPNS GYTGYAQKFQGRVTMTADRSTSTAYMELSSLRSEDTAVYYCAREVPETAAFEYWG QGTLVTVSS (SEQ ID NO: 420); and (b) a VL comprising the amino acid sequence of QSVLTQPPSVSGAPGQRVTISCTSSSSDIGAGLGVHWYQQLPGTAPKLLIEGYYNRPS GVPDRFSGSKSGTSASLTITGLLPEDEGDYYCQSWDGTLSALFGGGTKLTVLG (SEQ ID NO: 421).

In another embodiment, the anti-TL1A antibody antigen-binding fragment thereof comprises: (a) a heavy chain comprising the amino acid sequence of QVQLVQSGAEVKKPGASVKVSCKASGYTFTSYDINWVRQAPGQGLEWMGWLNPNS GYTGYAQKFQGRVTMTADRSTSTAYMELSSLRSEDTAVYYCAREVPETAAFEYWG QGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKKVEPKSCDK THTCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTI SKAKGQPREPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSLSLSPG (SEQ ID NO: 422); and (b) a light chain comprising the amino acid sequence of QSVLTQPPSVSGAPGQRVTISCTSSSSDIGAGLGVHWYQQLPGTAPKLLIEGYYNRPS GVPDRFSGSKSGTSASLTITGLLPEDEGDYYCQSWDGTLSALFGGGTKLTVLGQPKA APSVTLFPPSSEELQANKATLVCLISDFYPGAVTVAWKADSSPVKAGVETTTPSKQSN NKYAASSYLSLTPEQWKSHRSYSCQVTHEGSTVEKTVAPTECS (SEQ ID NO: 423).

The disclosure further provides anti-TL1A antibodies or antigen binding fragments described in U.S. Pat. Nos. 10,138,296 and 10,822,422, the disclosures of both of which are hereby incorporated in their entireties by reference.

The efficacy of the anti-TL1A antibodies in the preceding 4 paragraphs have been validated in animal and clinical studies as further described in U.S. Pat. Nos. 10,138,296; 10,822,422; clinicaltrialsregister.eu/ctr-search/trial/2020-001927-15/BG; Clarke, A W et al., MAbs. 2018 May-June; 10 (4): 664-677; the disclosures of all of which, including the anti-TL1A antibodies tested therein, the study design and study results, are incorporated hereby in their entireties by reference.
(b) siRNA against TL1A mRNA

Alternatively, the disclosure provides siRNA, shRNA, or other RNA/DNA modalities as TL1A inhibitor to reduce TL1A expression or reduce TL1A protein levels. In some embodiment, the TL1A inhibitor comprise the siRNA against TL1A mRNA described in Gonsky R. et al., Cytokine 63 (1): 36-42 (2013), which is hereby incorporated in its entirety by reference, and which siRNA reduces TL1A secretion by 50%, for example, when transfected into human monocytes. In another embodiment, the TL1A inhibitor comprise the siRNA against TL1A mRNA described in Yu M. et al., Mol Med Rep. 2016 April; 13 (4): 3265-72, which is hereby incorporated in its entirety by reference.
(c) DR3, DR3 variant proteins, Decoy Receptor 3 (“DcR3”) and DcR3 variant proteins

Additionally, the disclosure provides the soluble TL1A receptors or TL1A decoy receptors can compete with binding between TL1A and the native DR3 receptor for TL1A, and thus be used as TL1A inhibitors. In one embodiment, the TL1A inhibitor comprises a soluble DR3 protein. In another embodiment, the TL1A inhibitor comprises a variant of the soluble DR3 protein. In yet another embodiment, the TL1A inhibitor comprises a DR3-Fc fusion protein. In yet another embodiment, the TL1A inhibitor comprises a variant of the DR3-Fc fusion protein.

In some embodiments, the TL1A inhibitor comprises a DR3-Fc fusion protein comprising the sequence of DVDPASGTEAAAATPSKVWGSSAGRIEPRGGGRGALPTSMGQHGPSARARAGRAPG PRPAREASPRLRVHKTFKFVVVGVLLQVVPSSAATIKLHDQSIGTQQWEHSPLGELCP PGSHRSEHPGACNRCTEGVGYTNASNNLFACLPCTACKSDEEERSPCTTTRNTACQC KPGTFRNDNSAEMCRKCSRGCPRGMVKVKDCTPWSDIECVHKESGNGHNRGPIEPR GPTIKPCPPCKCPAPNLLGGPSVFIFPPKIKDVLMISLSPIVTCVVVDVSEDDPDVQISW FVNNVEVHTAQTQTHREDYNSTLRVVSALPIQHQDWMSGKEFKCKVNNKDLPAPIE RTISKPKGSVRAPQVYVLPPPEEEMTKKQVTLTCMVTDFMPEDIYVEWTNNGKTEL NYKNTEPVLDSDGSYFMYSKLRVEKKNWVERNSYSCSVVHEGLHNHHTTKSFSRTP GK (SEQ ID NO: 424). In certain embodiments, the TL1A inhibitor comprises a variant of the DR3-Fc fusion protein of SEQ ID NO: 424.

In some embodiments, the TL1A inhibitor comprises a soluble DR3 protein comprising the sequence of GGTRSPRCDCAGDFHKKIGLFCCRGCPAGHYLKAPCTEPCGNSTCLVCPQDTFLAWE NHHNSECARCQACDEQASQVALENCSAVADTRCGCKPGWFVECQVSQCVSSSPFYC QPCLDCGALHRHTRLLCSRRDTDCGTCLLGFYEHGDGCVSCPTSTLGSCPERCAAVC GWRQ (SEQ ID NO: 425). In another embodiment, the TL1A inhibitor comprises a soluble DR3 protein comprising the sequence of GGTRSPRCDCAGDFHKKIGLFCCRGCPAGHYLKAPCTEPCGNSTCLVCPQDTFLAWE NHHNSECARCQACDEQASQVALENCSAVADTRCGCKPGWFVECQVSQCVSSSPFYC QPCLDCGALHRHTRLLCSRRDTDCGTCLLGFYEHGDGCVSCPTS (SEQ ID NO: 490).

In one embodiment, the TL1A inhibitor comprises a DR3 protein comprising the sequence of SEQ ID NO:425 fused to an antibody Fc region. In another embodiment, the TL1A inhibitor comprises a DR3 protein comprising the sequence of SEQ ID NO:490 fused to an antibody Fc region. In one embodiment, the TL1A inhibitor comprises a variant of the soluble DR3 protein comprising the sequence of SEQ ID NO:425. In another embodiment, the TL1A inhibitor comprises a variant of the soluble DR3 protein comprising the sequence of SEQ ID NO: 490. In one embodiment, the TL1A inhibitor comprises a variant of a DR3 protein comprising the sequence of SEQ ID NO:425 fused to an antibody Fc region. In another embodiment, the TL1A inhibitor comprises a variant of a DR3 protein comprising the sequence of SEQ ID NO:490 fused to an antibody Fc region. As discussed above, such “variant” when used in relation to DR3 related proteins (such as soluble DR3 and DR3-Fc fusion) refers to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence.

More specifically, in some embodiments, the TL1A inhibitors comprise a variant DR3 or a variant DR3-Fc mutant of listed in Table 9C.

TABLE 9C
Variants of DR3 and DR3-Fc fusion

| | Mutations in SEQ ID Nos: 425 or | | | | | | |
|---|---|---|---|---|---|---|---|
| DR3 variants | 490 alone or when fused with Fc | | | | | | |
| DR3_variant_1 | E38K | V47P | W56R | A65T | K93A | Q101S | |
| DR3_variant_2 | H15Q | E34K | V47P | D51G | N61E | Q101S | |
| DR3_variant_3 | I18T | V47P | D51G | N61E | K93E | Q104P | L129P |
| DR3_variant_4 | I18T | V47P | D51G | A65T | K93A | Q101S | L129P |
| DR3_variant_5 | I18T | D51G | K93E | L129P | | | |
| DR3_variant_6 | H15Q | V47P | N61E | K93E | Q101S | Q104P | L129P |

The disclosure further provides that the TL1A-inhibitory effect of the DR3, DR3-Fc fusion, and the variants thereof of this Section 4.3.1(c) have been validated in studies as further described in Levin I et al., PLOS ONE 12 (3): e0173460. doi:10.1371/journal.pone.0173460, the disclosures of which, including the DR3, DR3-Fc fusion, and the variants thereof tested therein, the study design and study results, are incorporated hereby in their entireties by reference.

Similarly, the disclosure provides the soluble DcR3, a TL1A decoy receptor, can compete with binding between TL1A and the native DR3 receptor for TL1A, and thus be used as TL1A inhibitors. In one embodiment, the TL1A inhibitor comprises a soluble DcR3 protein. In another embodiment, the TL1A inhibitor comprises a variant of the soluble DcR3 protein. In yet another embodiment, the TL1A inhibitor comprises a DcR3-Fc fusion protein. In yet another embodiment, the TL1A inhibitor comprises a variant of the DcR3-Fc fusion protein.

In some embodiments, the TL1A inhibitor comprises the amino acid sequence of human DcR3 (accession number: NP_003814.1) or the DcR3 variants, or DcR3 fusion proteins thereof as described in WO2021049606A1, the disclosures of which, including the DcR3, DcR3-Fc fusion, and the variants thereof tested therein, the study design and study results, are incorporated hereby in their entireties by reference.

### 6.3.2 IL23 Inhibitors

In some embodiments, the IL23 inhibitors comprise anti-IL23 antibodies or antigen-binding fragments. In certain embodiments, the IL23 inhibitors are anti-IL23 antibodies or antigen-binding fragments. In some embodiments, the IL23 inhibitors consists of anti-IL23 antibodies or antigen-binding fragments. In some embodiments, the IL23 inhibitors for the combination therapy comprise any one selected from the group consisting of ustekinumab, guselkumab, risankizumab, brazikumab, mirikizumab, tildrakizumab, and briakinumab. In certain embodiments, the IL23 inhibitors comprise any one selected from the group consisting of variants of ustekinumab, variants of guselkumab, variants of risankizumab, variants of brazikumab, variants of mirikizumab, variants of tildrakizumab, and variants of briakinumab.

In certain embodiments, the IL23 inhibitors for the combination therapy comprise anti-IL23 antibodies as described in Section 4.3.2(h).

### (a) Ustekinumab

In some embodiments, the IL23 inhibitor provided herein for the combination therapy comprises ustekinumab. In some embodiments, the IL23 inhibitor provided herein for the combination therapy comprises a heavy chain variable region (VH) comprising the amino acid sequence of EVQLVQSGAEVKKPGESLKISCKGSGYSFTTYWLGWVRQMPGKGLDWIGIMSPVDS DIRYSPSFQGQVTMSVDKSITTAYLQWNSLKASDTAMYYCARRRPGQGYFDFWGQ GTLVTVSS (SEQ ID NO: 491) and a light chain variable region (VL) comprising the amino acid sequence of DIQMTQSPSSLSASVGDRVTITCRASQGISSWLAWYQQKPEKAPKSLIYAASSLQSGV PSRFSGSGSGTDFTLTISSLQPEDFATYYCQQYNIYPYTFGQGTKLEIKR (SEQ ID NO: 492). In some embodiments, the IL23 inhibitor provided herein for the combination therapy comprises a heavy chain variable region (VH) comprising the amino acid sequence set forth in SEQ ID NO: 424, a light chain variable region (VL) comprising the amino acid sequence set forth in SEQ ID NO: 425, and at least one pharmaceutically acceptable carrier or diluent.

In certain embodiments, the IL23 inhibitor provided herein for the combination therapy comprises a heavy chain CDR1 comprising the amino acid sequence of TYWLG (SEQ ID NO: 426), a heavy chain CDR2 comprising the amino acid sequence of IMSPVDSDIRYSPSFQ (SEQ ID NO: 427), a heavy chain CDR3 comprising the amino acid sequence of RRPGQGYFDF (SEQ ID NO: 428), a light chain CDR1 comprising the amino acid sequence of RASQGISSWLA (SEQ ID NO: 429), a light chain CDR2 comprising the amino acid sequence of AASSLQS (SEQ ID NO: 430), and a light chain CDR3 comprising an amino acid sequence of QQYNIYPYT (SEQ ID NO: 431) (such antibodies or antigen binding fragments, ustekinumab). In one embodiment, the IL23 inhibitor provided herein for the combination therapy comprises a heavy chain CDR1 comprising the amino acid sequence of SEQ ID NO: 426, a heavy chain CDR2 comprising the amino acid sequence of SEQ ID NO: 427, a heavy chain CDR3 comprising an amino acid sequence with not more than one conservative substitution from the amino acid sequence of SEQ ID NO: 428, a light chain CDR1 comprising the amino acid sequence of SEQ ID NO: 429, a light chain CDR2 comprising the amino acid sequence of SEQ ID NO: 430, and a light chain CDR3 comprising an amino acid sequence with not more than one conservative substitution from the amino acid sequence of SEQ ID NO: 431. In another embodiment, the IL23 inhibitor provided herein for the combination therapy comprises a heavy chain CDR1 comprising an amino acid sequence with not more than one conservative substitution from the amino acid sequence of SEQ ID NO: 426, a heavy chain CDR2 comprising an amino acid sequence with not more than one conservative substitution from the amino acid sequence of SEQ ID NO: 427, a heavy chain CDR3 comprising an amino acid sequence with not more than one conservative substitution from the amino acid sequence of SEQ ID NO: 428, a light chain CDR1 comprising an amino acid sequence with not more than one conservative substitution from the amino acid sequence of SEQ ID NO: 429, a light chain CDR2 comprising an amino acid sequence with not more than one conservative substitution from the amino acid sequence of SEQ ID NO: 430, and a light chain CDR3 comprising an amino acid sequence with not more than one conservative substitution from the amino acid sequence of SEQ ID NO: 431, wherein the antibody is a variant of the antibody comprising an heavy chain CDR1 amino acid sequence of SEQ ID NO: 426, the heavy chain CDR2 amino acid sequence of SEQ ID NO: 427, the heavy chain CDR3 amino acid sequence of SEQ ID NO: 428, the light chain CDR1 amino acid sequence of SEQ ID NO: 429, the light chain CDR2 amino acid sequence of SEQ ID NO: 430, and the light chain CDR3 amino acid sequence of SEQ ID NO: 431. In some embodiments, the anti-IL23 antibody or antigen-binding fragment further comprises a pharmaceutically acceptable carrier or diluent.

In one embodiment, the IL23 inhibitor provided herein for the combination therapy comprises a pharmaceutical composition comprising an effective amount of an anti-IL23 antibody or antigen-binding fragments having a heavy chain variable region and a light chain variable region, said heavy chain variable region comprising: a heavy chain CDR1 comprising the amino acid sequence of SEQ ID NO: 426, a heavy chain CDR2 comprising the amino acid sequence of SEQ ID NO: 427, a heavy chain CDR3 comprising the amino acid sequence of SEQ ID NO: 428, said light chain variable region comprising: a light chain CDR1 comprising the amino acid sequence of SEQ ID NO: 429, a light chain CDR2 comprising the amino acid sequence of SEQ ID NO: 430, and a light chain CDR3 comprising the amino acid sequence of SEQ ID NO: 431. In one embodiment, the IL23 inhibitor provided herein for the combination therapy comprises a pharmaceutical composition comprising an effective amount of an anti-IL23 antibody or antigen-binding fragments having a heavy chain variable region comprising the amino acid sequence of SEQ ID NO: 424 and a light chain variable region comprising the amino acid sequence of SEQ ID NO: 425. In one embodiment, the pharmaceutical composition further comprises histidine buffer, polysorbate 80 and sucrose. In one embodiment, the pH of the histidine buffer is about 6.0. In one embodiment, the effective amount in the pharmaceutical composition is 45 mg of the anti-IL23 antibody or antigen binding fragment. In one embodiment, the effective amount in the pharmaceutical composition is 90 mg of the anti-IL23 antibody or antigen binding fragment. In some embodiments, the effective amount is 0.001-50 mg/kilogram of said subject to whom the anti-IL23 antibody or antigen-binding fragment is administered. In certain embodiments, the anti-IL23 antibody or antigen binding fragment is administered by at least one mode selected from parenteral, subcutaneous, intravenous, intraabdominal, intracavitary, intracelial, intracolic, intragastric and buccal administration.

In some embodiments, the anti-IL23 inhibitors provided in this Section (Section 4.3.2(a)) is an anti-IL23 antibody or antigen-binding fragment.

In certain embodiments, In some embodiments, the anti-IL23 inhibitors provided in this Section (Section 4.3.2(a)) is an anti-IL23 antibody or antigen-binding fragment, wherein the anti-IL23 antibody or antigen-binding fragment also binds to IL12.

In some embodiments, the effective amount of the IL23 inhibitors in the combination therapy comprises administering an increasing dosing or maintenance interval. In some embodiments, the IL23 inhibitor of this Section (Section 4.3.2(a)) is administered in an initial dose, a dose 4 weeks after the initial dose and a dose once every 12 weeks for 24 weeks after administration of the initial dose and increasing the dosing interval 28 weeks after administration of the initial dose to a dosing interval of every 24 weeks after identifying the patient as a responder to the antibody 28 weeks after administration of the initial dose, wherein the dose is 45 mg or 90 mg. In certain embodiments, the effective amount of the IL23 inhibitors in the combination therapy comprises administering a pharmaceutical composition comprising an antibody or antigen-binding fragment to both IL-12 and IL-23 to a patient of an inflammatory disease or condition, wherein the antibody or antigen-binding fragment comprises a heavy chain variable amino acid sequence of SEQ ID NO: 424 and a light chain variable amino acid sequence of SEQ ID NO: 425, in an initial dose, a dose 4 weeks after the initial dose and a dose once every 12 weeks for 24 weeks after administration of the initial dose and increasing the dosing interval 28 weeks after administration of the initial dose to a dosing interval of every 24 weeks after identifying the patient as a responder to the antibody 28 weeks after administration of the initial dose, wherein the dose is 45 mg or 90 mg. In one embodiment, the pharmaceutical composition further comprises about 0.53 mg L-histidine per ml of the pharmaceutical composition; about 1.37 mg L-histidine monohydrochloride monohydrate per ml of the pharmaceutical composition; about 0.04 mg polysorbate 80 per ml of the pharmaceutical composition; about 76 mg of sucrose per ml of the pharmaceutical composition; and water as a diluent at standard state.

In certain embodiments, the effective amount of the IL23 inhibitors in the combination therapy comprises administering a pharmaceutical composition comprising an antibody or antigen-binding fragment thereof to both IL-12 and IL-23 to the subject having inflammatory disease or condition in an initial dose, a dose 4 weeks after the initial dose and a dose once every 12 weeks for 24 weeks after administration of the initial dose and increasing the dosing interval 28 weeks after administration of the initial dose to a dosing interval of every 24 weeks after identifying the patient as a responder to the antibody 28 weeks after administration of the initial dose, wherein the dose is 45 mg or 90 mg, and wherein the antibody or antigen-binding fragment comprises the heavy chain CDR1, CDR2, CDR3 amino acid sequences of SEQ ID NO: 426, SEQ ID NO: 427, and SEQ ID NO: 428, respectively; and the light chain CDR1, CDR2, CDR3 amino acid sequences of SEQ ID NO: 429, SEQ ID NO: 430, and SEQ ID NO: 430, respectively, and wherein the pharmaceutical composition further comprises about 0.53 mg L-histidine per ml of the pharmaceutical composition; about 1.37 mg L-histidine monohydrochloride monohydrate per ml of the pharmaceutical composition; about 0.04 mg polysorbate 80 per ml of the pharmaceutical composition; about 76 mg of sucrose per ml of the pharmaceutical composition; and water as a diluent at standard state.

In some embodiment, the IL23 inhibitor of this Section (Section 4.3.2(a)) is administered subcutaneously initially for one dose of 45 mg and 4 weeks later, followed by 45 mg administered subcutaneously every 12 weeks, if the subject of the administration has a body weight of equal to or less than 100 kilogram (kg) and is over the age of 17. In certain embodiment, the IL23 inhibitor of this Section (Section 4.3.2(a)) is administered subcutaneously initially for one dose of 90 mg and 4 weeks later, followed by 90 mg administered subcutaneously every 12 weeks, if the subject of the administration has a body weight of greater than 100 kg and is over the age of 17.

In some embodiment, the IL23 inhibitor of this Section (Section 4.3.2(a)) is administered subcutaneously initially for one dose of 0.75 mg/kg and 4 weeks later, followed by 0.75 mg/kg administered subcutaneously every 12 weeks, if the subject of the administration has a body weight of less than 60 kg and is under the age of 17. In certain embodiment, the IL23 inhibitor of this Section (Section 4.3.2(a)) is administered subcutaneously initially for one dose of 45 mg and 4 weeks later, followed by 45 mg administered subcutaneously every 12 weeks, if the subject of the administration has a body weight of between 60 and 100 kg (including 60 kg and 100 kg) and is under the age of 17. In certain embodiment, the IL23 inhibitor of this Section (Section 4.3.2(a)) is administered subcutaneously initially for one dose of 90 mg and 4 weeks later, followed by 90 mg administered subcutaneously every 12 weeks, if the subject of the administration has a body weight of greater than 100 kilogram and is under the age of 17.

In some embodiments of the combination therapy provided herein, the doses and/or the dosing regimens at which the IL23 inhibitor provided in this Section (Section 4.3.2(a)) is administered are the effective amount of the IL23 inhibitor of this Section (Section 4.3.2(a)) in the combination therapy.

In certain embodiments, the IL23 inhibitor comprises an anti-IL23 antibody or antigen binding fragment selected from those described in U.S. Pat. Nos. 6,902,734, 7,887,807, 8,703,141, 9,676,848, 11,078,267, 10,765,724, and 11,197,913, wherein the anti-IL23 antibody or antigen binding fragment also binds to IL12. In one embodiment, the IL23 inhibitor comprises an anti-IL23 antibody or antigen binding fragment selected from those described in U.S. Pat. Nos. 6,902,734, 7,887,807, 8,703,141, 9,676,848, 11,078,267, 10,765,724, and 11,197,913, at a formulation and dose as described in U.S. Pat. Nos. 6,902,734, 7,887,807, 8,703,141, 9,676,848, 11,078,267, 10,765,724, and 11,197,913, the disclosures of all of which are hereby incorporated in their entireties by reference.

In certain embodiments, the IL23 inhibitor comprises an anti-IL23 antibody or antigen binding fragment as described in the US FDA approved label for STELARA (revised December 2020, available at accessdata.fda.gov/drugsatfda_docs/label/2020/125261s154,761044s006lbl.pdf). In one embodiment, the IL23 inhibitor comprises an anti-IL23 antibody or antigen binding fragment as described in the US FDA approved label for STELARA (revised December 2020, available at accessdata.fda.gov/drugsatfda_docs/label/2020/125261s154,761044s006lbl.pdf), at a formulation and dose as described in the same US FDA approved label, the disclosures of all of which are hereby incorporated in their entireties by reference.

The disclosure further provides that the efficacy of the IL23 inhibitors of this Section (Section 4.3.2(a)) have been validated in studies as further described in U.S. Pat. Nos. 6,902,734, 7,887,807, 8,703,141, 9,676,848, 11078267, 10765724, and 11197913, and in clinical studies described in Sands B. E. et al., N Engl J Med 2019; 381:1201-1214 and the US FDA approved label for STELARA (revised December 2020, available at accessdata.fda.gov/drugsatfda_docs/label/2020/125261s154, 761044s006lbl.pdf) the disclosures of all of which are hereby incorporated in their entireties by reference.

Without being bound by the theory, ustekinumab is an antibody or antigen binding fragment to the p40 subunit of interleukin-12 (p35/p40, abbreviated as IL12 or IL-12) and interleukin-23 (p19/p40, abbreviated as IL23 or IL-23) and thus binds to both IL12 and IL23.

### (b) Guselkumab

In some embodiments, the IL23 inhibitor provided herein for the combination therapy comprises guselkumab. In some embodiments, the IL23 inhibitor provided herein for the combination therapy comprises an anti-IL23 antibody or antigen-binding fragment thereof comprising a light chain variable region and a heavy chain variable region, said light chain variable region comprising: a complementarity determining region light chain 1 (CDRL1) amino acid sequence of TGSSSNIGSGYDVH (SEQ ID NO: 435); a CDRL2 amino acid sequence of GNSKRPS (SEQ ID NO: 436); and a CDRL3 amino acid sequence of ASWTDGLSLVV (SEQ ID NO: 437), said heavy chain variable region comprising: a complementarity determining region heavy chain 1 (CDRH1) amino acid sequence of NYWIG (SEQ ID NO: 432); a CDRH2 amino acid sequence of IIDPSNSYTRYSPSFQG (SEQ ID NO: 433); and a CDRH3 amino acid sequence of WYYKPFDV (SEQ ID NO: 434) (such anti-IL23 antibodies or antigen-binding fragments, guselkumab). In certain embodiments, the anti-IL23 antibody or antigen-binding fragment binds to the P19 subunit of IL23. In some embodiments, the anti-IL23 antibody or antigen-binding fragment further comprises at least one human framework region adjacent to a complementarity determining region. In some embodiments, the anti-IL23 antibody or antigen-binding fragment binds IL-23p19 with at least one affinity selected from at least 10 M, at least 10⁻¹⁰ M, at least 10⁻¹¹ M, and at least 10⁻¹² M, at least 10⁻¹³ M, at least 10⁻¹⁴ M, and at least 10⁻¹⁵ M, as determined by surface plasmon resonance or the Kinexa method. In some embodiments, the anti-IL23 antibody or antigen-binding fragment substantially modulates an activity of the IL-23 polypeptide, the activity selected from the group consisting of binding to the IL-23 receptor (IL-23R), induction of STAT3 phosphorylation, and IL-17 production.

In some embodiments, the IL23 inhibitor provided herein for the combination therapy comprises an anti-IL23 antibody or antigen-binding fragment thereof comprising a light chain variable region comprising the amino acid sequence of QSVLTQPPSVSGAPGQRVTISCTGSSSNIGSGYDVHWYQQLPGTAPKLLIYGNSKRPS GVPDRFSGSKSGTSASLAITGLQSEDEADYYCASWTDGLSLVVFGGGTKLTVL (SEQ ID NO: 439) and a heavy chain variable region comprising the amino acid sequence of EVQLVQSGAEVKKPGESLKISCKGSGYSFSNYWIGWVRQMPGKGLEWMGIIDPSNS YTRYSPSFQGQVTISADKSISTAYLQWSSLKASDTAMYYCARWYYKPFDVWGQGTL VTVSS (SEQ ID NO: 438). In some embodiments, the IL23 inhibitor provided herein for the combination therapy comprises an anti-IL23 antibody or antigen-binding fragment thereof comprising (i) a heavy chain variable region comprising the amino acid sequence of SEQ ID NO: 438 having up to three substitutions from residues 50-66 of the amino acid sequence of SEQ ID NO: 438 and (ii) a light chain variable region comprising the amino acid sequence of SEQ ID NO:439. In some embodiments, the anti-IL23 antibody or antigen-binding fragment binds IL-23p19 with at least one affinity selected from at least 10⁻⁹ M, at least 10⁻¹⁰ M, at least 10⁻¹¹ M, and at least 10⁻¹² M, at least 10⁻¹³ M, at least 10⁻¹⁴ M, and at least 10⁻¹⁵ M, as determined by surface plasmon resonance or the Kinexa method. In some embodiments, the anti-IL23 antibody or antigen-binding fragment substantially modulates an activity of the IL-23 polypeptide, the activity selected from the group consisting of binding to the IL-23 receptor (IL-23R), induction of STAT3 phosphorylation, and IL-17 production.

In some embodiments, the IL23 inhibitor provided in this Section (Section 4.3.2(b)) for the combination therapy is in a pharmaceutical composition further comprising at least one pharmaceutically acceptable carrier or diluent. In some embodiments, the IL23 inhibitor provided in this Section (Section 4.3.2(b)) for the combination therapy is formulated for a parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal delivery device or system.

In one embodiment, the IL23 inhibitor provided herein for the combination therapy comprises a pharmaceutical composition comprising an effective amount of an anti-IL23 antibody or antigen-binding fragments comprising a light chain variable region and a heavy chain variable region, said light chain variable region comprising: a complementarity determining region light chain 1 (CDRL1) amino acid sequence of TGSSSNIGSGYDVH (SEQ ID NO: 435); a CDRL2 amino acid sequence of GNSKRPS (SEQ ID NO: 436); and a CDRL3 amino acid sequence of ASWTDGLSLVV (SEQ ID NO: 437), said heavy chain variable region comprising: a complementarity determining region heavy chain 1 (CDRH1) amino acid sequence of NYWIG (SEQ ID NO: 432); a CDRH2 amino acid sequence of IIDPSNSYTRYSPSFQG (SEQ ID NO: 433); and a CDRH3 amino acid sequence of WYYKPFDV (SEQ ID NO: 434). In one embodiment, the IL23 inhibitor provided herein for the combination therapy comprises a pharmaceutical composition comprising an effective amount of an anti-IL23 antibody or antigen-binding fragments comprising a light chain variable region comprising the amino acid sequence of SEQ ID NO: 439 and a heavy chain variable region comprising the amino acid sequence of SEQ ID NO: 438. In one embodiment, the effective amount in the pharmaceutical composition is about 0.001-50 mg/kilogram of the subject to whom the anti-IL23 antibody or antigen binding fragment is administered. In certain embodiments, the anti-IL23 antibody or antigen binding fragment is administered by at least one mode selected from parenteral, subcutaneous, intramuscular, intravenous, intraarticular, intrabronchial, intraabdominal, intracapsular, intracartilaginous, intracavitary, intrapelvic, intraperitoneal, intrapleural, intrapulmonary, intrasynovial, intrathoracic, intralesional, bolus, intranasal, and transdermal. In one embodiment, the pharmaceutical composition further comprises a histidine buffer. In one embodiment, the pharmaceutical composition further comprises sucrose. In one embodiment, the pharmaceutical composition further comprises polysorbate 80. In one embodiment, the pharmaceutical composition further comprises histidine buffer, polysorbate 80 and sucrose. In one embodiment, the pH of the histidine buffer is about 5.8. In certain embodiment, the pharmaceutical composition is formulated in a prefilled syringe.

In certain embodiments, the antibody fragment is a Fab, Fab′, F(ab′)₂, facb, pFc′, Fd, Fv or scFv.

In certain embodiments, the effective amount of the IL23 inhibitors in the combination therapy comprises a pharmaceutical composition comprising an anti-IL23 antibody or antigen-binding fragment at a dose of 100 mg administered in an initial dose, 4 weeks after the initial dose and every 8 weeks after the dose at 4 weeks, wherein the anti-IL-23 specific antibody or antigen-binding fragment comprises a light chain variable region comprising the amino acid sequence of SEQ ID NO: 439 and a heavy chain variable region comprising the amino acid sequence of SEQ ID NO: 438, wherein the pharmaceutical composition comprising or consisting of: 100 mg/mL of the anti-IL-23 specific antibody or antigen-binding fragment; 7.9% (w/v) sucrose; 4.0 mM Histidine; 6.9 mM L-Histidine monohydrochloride monohydrate; 0.053% (w/v) Polysorbate 80; and water as a diluent.

In certain embodiments, the IL23 inhibitor of this Section (Section 4.3.2(b)) is administered to a subject having an inflammatory disease or condition in an effective amount at a dose of 100 mg in an initial dose, 4 weeks after the initial dose and every 8 weeks after the dose at 4 weeks.

In certain embodiments, the IL23 inhibitor of this Section (Section 4.3.2(b)) binds to the p19 subunit of the IL23. In certain embodiments, the IL23 inhibitor of this Section (Section 4.3.2(b)) binds to IL23 but does not bind to IL12.

In certain embodiments, the IL23 inhibitor of this Section (Section 4.3.2(b)) is administered at 100 mg per dose by subcutaneous injection at Week 0, Week 4 and every 8 weeks thereafter.

In some embodiments of the combination therapy provided herein, the doses and/or the dosing regimens at which the IL23 inhibitor provided in this Section (Section 4.3.2(b)) is administered are the effective amount of the IL23 inhibitor of this Section (Section 4.3.2(b)) in the combination therapy.

In certain embodiments, the IL23 inhibitor comprises guselkumab or a variant of guselkumab selected from those described in U.S. Pat. Nos. 7,935,344, 7,993,645, 8,221,760, 9,783,607, 10,954,297, and 11,208,474. In one embodiment, the IL23 inhibitor comprises guselkumab or a variant of guselkumab selected from those described in U.S. Pat. Nos. 7,935,344, 7,993,645, 8,221,760, 9,783,607, 10,954,297, and 11,208,474, at a formulation and dose as described in U.S. Pat. Nos. 7,935,344, 7,993,645, 8,221,760, 9,783,607, 10,954,297, and 11,208,474, the disclosures of all of which are hereby incorporated in their entireties by reference.

In certain embodiments, the IL23 inhibitor comprises guselkumab as described in the US FDA approved label for TREMFYA (revised July 2020, available at accessdata.fda.gov/drugsatfda_docs/label/2020/761061s007lbl.pdf). In one embodiment, the IL23 inhibitor comprises guselkumab as described in the US FDA approved label for TREMFYA (revised July 2020, available at accessdata.fda.gov/drugsatfda_docs/label/2020/761061s007lbl.pdf), at a formulation and dose as described in the same US FDA approved label, the disclosures of all of which are hereby incorporated in their entireties by reference.

The disclosure further provides that the IL23 inhibitory effects of the antibody or antigen-binding fragment of this Section (Section 4.3.2(b)) have been validated in studies as further described in U.S. Pat. Nos. 7,935,344, 7,993,645, 8,221,760, 9,783,607, 10954297, and 11208474, and in clinical studies described in Danese S et al., Journal of Crohn's and Colitis, Volume 15, Issue Supplement_1, May 2021, Pages S027-S028 and in the US FDA approved label for TREMFYA (revised July 2020, available at accessdata.fda.gov/drugsatfda_docs/label/2020/761061s007lbl.pdf) the disclosures of all of which are hereby incorporated in their entireties by reference.

Without being bound by the theory, the disclosure provides that guselkumab binds to the p19 subunit of IL23 and inhibit IL23 function (antagonist of IL23).

### (c) Risankizumab

In some embodiments, the IL23 inhibitor provided herein for the combination therapy comprises risankizumab. In some embodiments, In some embodiments, the IL23 inhibitor provided herein for the combination therapy comprises an anti-interleukin (IL)-23 antibody or antigen-binding fragment thereof, wherein the antibody or antigen-binding fragment thereof comprises: (a) a light chain variable region comprising the amino acid sequence of KASRDVAIAVA (SEQ ID NO: 440) (CDRL1); the amino acid sequence of WASTRHT (SEQ ID NO: 441) (CDRL2); and the amino acid sequence of HQYSSYPFT (SEQ ID NO: 442) (CDRL3); and (b) a heavy chain variable region comprising the amino acid sequence of GNTFTDQTIH (SEQ ID NO: 446), GYTFTDQTIH (SEQ ID NO: 443), GFTFTDQTIH (SEQ ID NO: 447), or GGTFTDQTIH (SEQ ID NO: 448) (CDRH1); the amino acid sequence of YIYPRDDSPKYNENFKG (SEQ ID NO: 444) (CDRH2); and the amino acid sequence of PDRSGYAWFIY (SEQ ID NO: 445) (CDRH3). In some embodiments, the IL23 inhibitor provided herein for the combination therapy comprises an anti-IL23 antibody or antigen-binding fragment thereof, wherein the antibody or antigen-binding fragment thereof comprises: (a) a light chain variable region comprising the amino acid sequence of KASRDVAIAVA (SEQ ID NO: 440) (CDRL1); the amino acid sequence of WASTRHT (SEQ ID NO: 441) (CDRL2); and the amino acid sequence of HQYSSYPFT (SEQ ID NO: 442) (CDRL3); and (b) a heavy chain variable region comprising the amino acid sequence of GYTFTDQTIH (SEQ ID NO: 443) (CDRH1); the amino acid sequence of YIYPRDDSPKYNENFKG (SEQ ID NO: 444) (CDRH2); and the amino acid sequence of PDRSGYAWFIY (SEQ ID NO: 445) (CDRH3) (such antibodies or antigen binding fragments risankizumab). In some embodiments, the IL23 inhibitor provided herein for the combination therapy comprises an anti-IL23 antibody or antigen-binding fragment thereof, wherein the antibody or antigen-binding fragment thereof comprises a light chain variable region comprising the amino acid sequence of DIQMTQSPSSLSASVGDRVTITCKASRDVAIAVAWYQQKPGKVPKLLIYWASTRHTG VPSRFSGSGSRTDFTLTISSLQPEDVADYFCHQYSSYPFTFGSGTKLEIK (SEQ ID NO: 449) and a heavy chain variable region comprising the amino acid sequence of QVQLVQSGAEVKKPGSSVKVSCKASGYTFTDQTIHWMRQAPGQGLEWIGYIYPRDD SPKYNENFKGKVTITADKSTSTAYMELSSLRSEDTAVYYCAIPDRSGYAWFIYWGQG TLVTVSS (SEQ ID NO: 450). In some embodiments, the IL23 inhibitor provided herein for the combination therapy comprises an anti-IL23 antibody or antigen-binding fragment thereof, wherein the antibody or antigen-binding fragment thereof comprises a light chain variable region comprising the amino acid sequence of SEQ ID NO: 449 and a heavy chain variable region comprising the amino acid sequence of QVQLVQSGAEVKKPGSSVKVSCKASGFTFTDQTIHWVRQAPGQGLEWMGYIYPRD DSPKYNENFKGKVTLTADKSTSTAYMELSSLRSEDTAVYYCAIPDRSGYAWFIYWG QGTLVTVSS (SEQ ID NO: 451). In some embodiments, the IL23 inhibitor provided herein for the combination therapy comprises an anti-IL23 antibody or antigen-binding fragment thereof, wherein the antibody or antigen-binding fragment thereof comprises a light chain variable region comprising the amino acid sequence of DIQMTQSPSSLSASVGDRVTITCKASRDVAIAVAWYQQKPGKVPKLLLFWASTRHTG VPDRFSGSGSGTDFTLTISSLQPEDLADYYCHQYSSYPFTFGQGTKLEIK (SEQ ID NO: 452) and a heavy chain variable region comprising the amino acid sequence of SEQ ID NO: 451. In some embodiments, the IL23 inhibitor provided herein for the combination therapy comprises an anti-IL23 antibody or antigen-binding fragment thereof, wherein the antibody or antigen-binding fragment thereof comprises a light chain variable region comprising the amino acid sequence of SEQ ID NO: 452 and a heavy chain variable region comprising the amino acid sequence of SEQ ID NO: 450. In some embodiments, the IL23 inhibitor provided herein for the combination therapy comprises an anti-IL23 antibody or antigen-binding fragment thereof, wherein variable light chain of the antibody or antigen-binding fragment thereof is linked to a human kappa light chain constant region and the variable heavy chain of the antibody or antigen-binding fragment thereof is linked to a human IgG1 heavy chain constant region.

In certain embodiments, the IL23 inhibitor provided herein for the combination therapy comprises an anti-IL23 antibody or antigen-binding fragment thereof, wherein the antibody or antigen-binding fragment thereof comprises (a) a humanized light chain variable domain comprising the CDRs of SEQ ID NO: 449 or 452 and framework regions having an amino acid sequence at least 90% identical to the amino acid sequence of the framework regions of the variable domain light chain amino acid sequence of SEQ ID NO: 449 or 452; and (b) a humanized heavy chain variable domain comprising the CDRs of SEQ ID NO: 450 or 451 and framework regions having an amino acid sequence at least 90% identical to the amino acid sequence of the framework regions of the variable domain heavy chain amino acid sequence of SEQ ID NO: 450 or 451. In certain embodiments, the IL23 inhibitor provided herein for the combination therapy comprises an anti-IL23 antibody or antigen-binding fragment thereof, wherein the antibody or antigen-binding fragment thereof comprises (a) a humanized light chain variable domain comprising the CDRs of SEQ ID NO: 449 and framework regions having an amino acid sequence at least 90% identical to the amino acid sequence of the framework regions of the variable domain light chain amino acid sequence of SEQ ID NO: 449; and (b) a humanized heavy chain variable domain comprising the CDRs of SEQ ID NO: 450 and framework regions having an amino acid sequence at least 90% identical to the amino acid sequence of the framework regions of the variable domain heavy chain amino acid sequence of SEQ ID NO: 450.

In some embodiments, the IL23 inhibitor provided herein for the combination therapy comprises an anti-IL23 antibody or antigen-binding fragment thereof, wherein the antibody comprises a light chain comprising an amino acid sequence of DIQMTQSPSSLSASVGDRVTITCKASRDVAIAVAWYQQKPGKVPKLLIYWASTRHTG VPSRFSGSGSRTDFTLTISSLQPEDVADYFCHQYSSYPFTFGSGTKLEIKRTVAAPSVFI FPPSDEQLKSGTASVVCLLNNFYPREAKVQWKVDNALQSGNSQESVTEQDSKDSTY SLSSTLTLSKADYEKHKVYACEVTHQGLSSPVTKSFNRGEC (SEQ ID NO: 453), and a heavy chain comprising an amino acid sequence of QVQLVQSGAEVKKPGSSVKVSCKASGYTFTDQTIHWMRQAPGQGLEWIGYIYPRDD SPKYNENFKGKVTITADKSTSTAYMELSSLRSEDTAVYYCAIPDRSGYAWFIYWGQG TLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKRVEPKSCDKTHT CPPCPAPEAAGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISK AKGQPREPQVYTLPPSREEMTKNQVSLTCLVKGFYPSDIAVEWESNGQPENNYKTTP PVLDSDGSFFLYSKLTVDKSRWQQGNVFSCSVMHEALHNHYTQKSLSLSPG (SEQ ID NO: 454).

In some embodiments, the IL23 inhibitor provided in this Section (Section 4.3.2(c)) for the combination therapy is in a pharmaceutical composition further comprising a pharmaceutically acceptable carrier.

In some embodiments, the IL23 inhibitor provided in this Section (Section 4.3.2(c)) for the combination therapy is formulated in a pharmaceutical composition of 150 mg/mL anti-IL23 antibody or antigen-binding fragment thereof provided in this Section (Section 4.3.2(c)) in a single-dose prefilled pen. In one embodiment, the IL23 inhibitor provided in this Section (Section 4.3.2(c)) for the combination therapy is formulated in a pharmaceutical composition of 75 mg/0.83 mL anti-IL23 antibody or antigen-binding fragment thereof provided in this Section (Section 4.3.2(c)) in a single-dose prefilled pen.

In some embodiments, the IL23 inhibitor provided in this Section (Section 4.3.2(c)) for the combination therapy is formulated in a pharmaceutical composition, wherein the pharmaceutical composition comprises 150 mg/mL anti-IL23 antibody or antigen-binding fragment thereof provided in this Section (Section 4.3.2(c)), acetic acid (0.054 mg), polysorbate 20 (0.2 mg), sodium acetate trihydrate (1.24 mg), trehalose dihydrate (70 mg), and Water for Injection, USP, wherein the pH is 5.7.

In certain embodiments, the IL23 inhibitor provided in this Section (Section 4.3.2(c)) for the combination therapy is formulated in a pharmaceutical composition, wherein the pharmaceutical composition comprises 75 mg/0.83 mL anti-IL23 antibody or antigen-binding fragment thereof provided in this Section (Section 4.3.2(c)), disodium succinate hexahydrate (0.88 mg), polysorbate 20 (0.17 mg), sorbitol (34 mg), succinic acid (0.049 mg), and Water for Injection, USP, wherein the pH is 6.2.

In certain embodiments, the effective amount of the IL23 inhibitors in the combination therapy comprises a pharmaceutical composition comprising an anti-IL23 antibody or antigen-binding fragment thereof provided in this Section (Section 4.3.2(c)) at a dose of 150 mg by subcutaneous injection at Week 0, Week 4, and every 12 weeks thereafter. In some embodiments, the effective amount of the IL23 inhibitors in the combination therapy comprises an anti-IL23 antibody or antigen-binding fragment thereof provided in this Section (Section 4.3.2(c)) at a dose of 150 mg by subcutaneous injection at Week 0, Week 4, and every 12 weeks thereafter.

In some embodiments, the anti-IL23 antibody or antigen-binding fragment thereof provided in this Section (Section 4.3.2(c)) for the combination therapy is administered at a dose of 150 mg by subcutaneous injection at Week 0, Week 4, and every 12 weeks thereafter.

In some embodiments of the combination therapy provided herein, the doses and/or the dosing regimens at which the IL23 inhibitor provided in this Section (Section 4.3.2(c)) is administered are the effective amount of the IL23 inhibitor of this Section (Section 4.3.2(c)) in the combination therapy.

In certain embodiments, the IL23 inhibitor comprises an anti-IL23 antibody or antigen-binding fragment selected from those described in U.S. Pat. Nos. 8,778,346, 9,441,036, and 10,202,448. In one embodiment, the IL23 inhibitor comprises an anti-IL23 antibody or antigen-binding fragment selected from those described in U.S. Pat. Nos. 8,778,346, 9,441,036, and 10,202,448, at a formulation and dose as described in U.S. Pat. Nos. 8,778,346, 9,441,036, and 10,202,448, the disclosures of all of which are hereby incorporated in their entireties by reference.

In certain embodiments, the IL23 inhibitor comprises risankizumab as described in the US FDA approved label for SKYRIZI (revised April 2021, available at accessdata.fda.gov/scripts/cder/daf/index.cfm?event-overview.process&ApplNo-761105). In one embodiment, the IL23 inhibitor comprises risankizumab as described in the US FDA approved label for SKYRIZI (revised April 2021, available at accessdata.fda.gov/scripts/cder/daf/index.cfm?event-overview.process&ApplNo=761105), at a formulation and dose as described in the same US FDA approved label, the disclosures of all of which are hereby incorporated in their entireties by reference.

The disclosure further provides that the IL23 inhibitory effects of the antibody or antigen-binding fragment of this Section (Section 4.3.2(c)) have been validated in studies as further described in U.S. Pat. Nos. 8,778,346, 9,441,036, and 10,202,448, and in clinical studies described in Feagan B. G. et al., Lancet Gastroenterol Hepatol. 2018 October; 3 (10): 671-680, and in the US FDA approved label for SKYRIZI (revised April 2021, available at accessdata.fda.gov/scripts/cder/daf/index.cfm?event-overview.process&ApplNo=761105) the disclosures of all of which are hereby incorporated in their entireties by reference.

In certain embodiments, the IL23 inhibitor of this Section (Section 4.3.2(c)) binds to the p19 subunit of the IL23. In certain embodiments, the IL23 inhibitor of this Section (Section 4.3.2(c)) binds to IL23 but does not bind to IL12.

Without being bound by the theory, the disclosure provides that risankizumab binds to the p19 subunit of IL23 and inhibit IL23 function (antagonist of IL23).

### (d) Brazikumab

In some embodiments, the IL23 inhibitor provided herein for the combination therapy comprises brazikumab. In some embodiments, the IL23 inhibitor provided herein for the combination therapy comprises an anti-IL23 antibody or antigen-binding fragment thereof, wherein the antibody or antigen-binding fragment thereof comprises: (a) a heavy chain variable region comprising a CDRH1 comprising the amino acid sequence of SYGMH (SEQ ID NO: 455), a CDRH2 comprising the amino acid sequence of VIWYDGSNEYYADSVKGR (SEQ ID NO: 456), and a CDRH3 comprising the amino acid sequence of DRGYTSSWYPDAFDI (SEQ ID NO: 457); and (b) a light chain variable region comprising a CDRL1 comprising the amino acid sequence of TGSSSNTGAGYDVH (SEQ ID NO: 458), a CDRL2 comprising the amino acid sequence of GSGNRPS (SEQ ID NO: 459), and a CDRL3 comprising the amino acid sequence of QSYDSSLSGWV (SEQ ID NO: 460) (such anti-IL23 antibodies and antigen binding fragments thereof, brazikumab). In some embodiments, the IL23 inhibitor provided herein for the combination therapy comprises an anti-IL23 antibody or antigen-binding fragment thereof, wherein the antibody or antigen-binding fragment thereof comprises a heavy chain variable region comprising the amino acid sequence of QVQLVESGGGVVQPGRSLRLSCAASGFTFSSYGMHWVRQAPGKGLEWVAVIWYDG SNEYYADSVKGRFTISRDNSKNTLYLQMNSLRAEDTAVYYCARDRGYTSSWYPDAF DIWGQGTMVTVSS (SEQ ID NO: 461) and a light chain variable region comprising the amino acid sequence of QSVLTQPPSVSGAPGQRVTISCTGSSSNTGAGYDVHWYQQVPGTAPKLLIYGSGNRP SGVPDRFSGSKSGTSASLAITGLQAEDEADYYCQSYDSSLSGWVFGGGTRLTVL (SEQ ID NO: 462). In some embodiments, the IL23 inhibitor provided herein for the combination therapy comprises an anti-IL23 antibody or antigen-binding fragment thereof, wherein the antibody or antigen-binding fragment thereof comprises a heavy chain variable region comprising amino acid residues 31-35, 50-65 and 99-113 of SEQ ID NO: 461; and a light chain variable region comprising amino acid residues 23-36, 52-58 and 91-101 of SEQ ID NO: 462.

In certain embodiments, the anti-IL23 antibody or antigen-binding fragment thereof provided herein including in this Section (Section 4.3.2(d)) has at least one property selected from the group consisting of: (a) reducing human IL-23 activity; (b) reducing production of a proinflammatory cytokine; (c) binding to human IL-23 with a KD of less than or equal to 5×10⁻⁸ M; (d) having a k_{off} rate of less than or equal to 5×10⁻⁶ s⁻¹; and (e) having an IC₅₀ of less than or equal to 400 pM.

In some embodiments, the IL23 inhibitor provided in this Section (Section 4.3.2(d)) for the combination therapy is in a pharmaceutical composition further comprising a pharmaceutically acceptable excipient.

In some embodiments, the subject in the combination therapy provided herein receives a plurality of intravenous administrations of the IL23 inhibitor provided in this Section (Section 4.3.2(d)), a plurality of subcutaneous administrations of the IL23 inhibitor provided in this Section (Section 4.3.2(d)), or both. In some embodiments, the intravenous administrations are delivered within 4 weeks of initiating treatment. In some embodiments, the subcutaneous administrations are delivered at least 12 weeks after initiating treatment. In some embodiments, the subcutaneous administrations are delivered on about day 85 and about every 4 weeks thereafter.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered in an amount and at an interval of: (a) 720-1440 mg on or about days 1, 29, and 57 delivered intravenously, followed by (b) about 240 mg delivered subcutaneously on or about day 85 and about every 4 weeks thereafter through at least week 48.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered by intravenous infusion. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered at a total dosage of at least 700 mg, at least 1400 mg, at least 2100 mg, or at least 4200 mg of the anti-IL23 antibody or antigen-binding fragment thereof. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered by intravenous infusion comprising at least 70 mg of the anti-IL23 antibody or antigen binding fragment in a volume of about 100 ml delivered over a period of at least 30 minutes. In certain embodiment, the IL23 inhibitor comprises the anti-IL23 antibody or antigen-binding fragment thereof provided in this Section (Section 4.3.2(d)) and the anti-IL23 antibody or antigen-binding fragment thereof is administered by a plurality of intravenous infusions. In certain embodiments, the plurality of intravenous infusions of this paragraph each comprises the same quantity of anti-IL-23 antibody.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered by intravenous (IV) infusion at Weeks 0 and 4 followed subcutaneous (SC) injection at Weeks 8 and 12 in the induction phase and at Weeks 16, 20 and 24 in the maintenance phase. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered by subcutaneous (SC) at Weeks 0 and 4 followed SC injection at Weeks 8 and 12 in the induction phase and at Weeks 16, 20 and 24 in the maintenance phase. In some embodiment of the combination therapy of this Section (Section 4.3.2(d)), the subject received 210 mg of the anti-IL23 antibody or antigen-binding fragment thereof provided in this Section (Section 4.3.2(d)) by SC injection every 4 weeks after 24 week and up to Week 48.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered by IV infusion at a dose of 700 mg at Weeks 0 and 4 followed by 210 mg SC injection of the anti-IL23 antibody or antigen-binding fragment thereof at Weeks 8 and 12 in the induction phase. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered by SC injection at a dose of 700 mg at Weeks 0 and 4 followed by 210 mg SC injection of the anti-IL23 antibody or antigen-binding fragment thereof at Weeks 8 and 12 in the induction phase. In some embodiment of the combination therapy of this Section (Section 4.3.2(d)), the subject received 210 mg of the anti-IL23 antibody or antigen-binding fragment thereof provided in this Section (Section 4.3.2(d)) by SC injection every 4 weeks in the maintenance phase up to Week 48.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered subcutaneously. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered in a plurality of doses. In some embodiments of the combination therapy provided herein, the IL23 inhibitor comprises the anti-IL23 antibody or antigen-binding fragment thereof provided in this Section (Section 4.3.2(d)) and a total dosage of at least 105 mg or at least 210 mg of the anti-IL23 antibody or antigen-binding fragment thereof is administered. In some embodiments of the combination therapy provided herein, the IL23 inhibitor comprises the anti-IL23 antibody or antigen-binding fragment thereof provided in this Section (Section 4.3.2(d)) and each dose of the anti-IL23 antibody or antigen-binding fragment thereof comprises about 70 mg of the anti-IL23 antibody or antigen-binding fragment thereof. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered in a plurality of doses, wherein a second dose is administered about two weeks after a first dose, and a third and subsequent doses are administered about four weeks after a preceding dose. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered in a plurality of doses, wherein the plurality of doses is about 10 doses. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered in a plurality of doses, wherein a second dose is administered about two weeks after a first dose, and a third and subsequent doses are administered about four weeks after a preceding dose, and wherein the first and second doses are administered by intravenous infusion and any subsequent dose is administered subcutaneously. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered in a plurality of doses, wherein the plurality of doses is about 10 doses, and wherein each dose comprises at least 70 mg of anti-IL-23 antibody.

In some embodiments of the combination therapy provided herein, the doses and/or the dosing regimens at which the IL23 inhibitor provided in this Section (Section 4.3.2(d)) is administered are the effective amount of the IL23 inhibitor of this Section (Section 4.3.2(d)) in the combination therapy.

In certain embodiments, the IL23 inhibitor comprises an anti-IL23 antibody or antigen binding fragment selected from those described in U.S. Pat. Nos. 8,722,033, 9,487,580, and 9,951,129 and US Publication Nos. US20210277105A1 (U.S. Ser. No. 17/259,448) and US20210079086A1 (U.S. Ser. No. 16/999,470). In one embodiment, the IL23 inhibitor comprises an ozanimod or a derivative of ozanimod selected from those described in U.S. Pat. Nos. 8,722,033, 9,487,580, and 9,951,129 and US Publication Nos. US20210277105A1 (U.S. Ser. No. 17/259,448) and US20210079086A1 (U.S. Ser. No. 16/999,470), at a formulation and dose as described in U.S. Pat. Nos. 8,722,033, 9,487,580, and 9,951,129 and US Publication Nos. US20210277105A1 (U.S. Ser. No. 17/259,448) and US20210079086A1 (U.S. Ser. No. 16/999,470), the disclosures of all of which are hereby incorporated in their entireties by reference.

In certain embodiments, the IL23 inhibitor comprises an anti-IL23 antibody or antigen binding fragment selected from those described in the clinical study NCT02574637 (available at clinicaltrials.gov/ct2/show/NCT02574637). In one embodiment, the IL23 inhibitor comprises an anti-IL23 antibody or antigen binding fragment selected from those described in the clinical study NCT02574637 (available at clinicaltrials.gov/ct2/show/NCT02574637), at a formulation and dose as described in the same clinical study, the disclosures of all of which are hereby incorporated in their entireties by reference.

The disclosure further provides that the IL23 inhibitory effects of the antibody or antigen-binding fragment of this Section (Section 4.3.2(d)) have been validated in studies as further described in U.S. Pat. Nos. 8,722,033, 9,487,580, and 9,951,129 and US Publication Nos. US20210277105A1 (U.S. Ser. No. 17/259,448) and US20210079086A1 (U.S. Ser. No. 16/999,470), and in clinical studies described in Sands B. E. et al., Gastroenterology 2017 July; 153 (1): 77-86.e6, and in the clinical study NCT02574637 (available at clinicaltrials.gov/ct2/show/NCT02574637), the disclosures of all of which are hereby incorporated in their entireties by reference.

In certain embodiments, the IL23 inhibitor of this Section (Section 4.3.2(d)) binds to the p19 subunit of the IL23. In certain embodiments, the IL23 inhibitor of this Section (Section 4.3.2(d)) binds to IL23 but does not bind to IL12.

Without being bound by the theory, the disclosure provides the disclosure provides that brazikumab binds to the p19 subunit of IL23 and inhibit IL23 function (antagonist of IL23).

### (e) Mirikizumab

In some embodiments, the IL23 inhibitor provided herein for the combination therapy comprises mirikizumab. In some embodiments, the IL23 inhibitor provided herein for the combination therapy comprises an anti-IL23 antibody or antigen-binding fragment thereof, wherein the antibody or antigen-binding fragment thereof comprises: (a) a light chain variable region comprising a CDRL1 comprising the amino acid sequence of KASDHILKFLT (SEQ ID NO: 463), a CDRL2 comprising the amino acid sequence of GATSLET (SEQ ID NO: 464), and a CDRL3 comprising the amino acid sequence of QMYWSTPFT (SEQ ID NO: 465); and (b) a heavy chain variable region comprising a CDRH1 comprising the amino acid sequence of GYKFTRYVMH (SEQ ID NO: 466), a CDRH2 comprising the amino acid sequence of YINPYNDGTNYNEKFKG (SEQ ID NO: 467), and a CDRH3 comprising the amino acid sequence of ARNWDTGL (SEQ ID NO: 468). In some embodiments, the IL23 inhibitor provided herein for the combination therapy comprises an anti-IL23 antibody or antigen-binding fragment thereof, wherein the antibody or antigen-binding fragment thereof comprises a heavy chain variable region comprising the amino acid sequence of QVQLVQSGAEVKKPGSSVKVSCKASGYKFTRYVMHWVRQAPGQGLEWMGYINPY NDGTNYNEKFKGRVTITADKSTSTAYMELSSLRSEDTAVYYCARNWDTGLWGQGT TVTVSS (SEQ ID NO: 469) and a light chain variable region comprising the amino acid sequence of DIQMTQSPSSLSASVGDRVTITCKASDHILKFLTWYQQKPGKAPKLLIYGATSLETGV PSRFSGSGSGTDFTLTISSLQPEDFATYYCQMYWSTPFTFGGGTKVEIK (SEQ ID NO: 470).

In some embodiments, the IL23 inhibitor provided herein for the combination therapy comprises an anti-IL23 antibody or antigen-binding fragment thereof, wherein the antibody comprises a heavy chain comprising the amino acid sequence of QVQLVQSGAEVKKPGSSVKVSCKASGYKFTRYVMHWVRQAPGQGLEWMGYINPY NDGTNYNEKFKGRVTITADKSTSTAYMELSSLRSEDTAVYYCARNWDTGLWGQGT TVTVSSASTKGPSVFPLAPCSRSTSESTAALGCLVKDYFPEPVTVSWNSGALTSGVHT FPAVLQSSGLYSLSSVVTVPSSSLGTKTYTCNVDHKPSNTKVDKRVESKYGPPCPPCP APEAAGGPSVFLFPPKPKDTLMISRTPEVTCVVVDVSQEDPEVQFNWYVDGVEVHN AKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDS DGSFFLYSRLTVDKSRWQEGNVFSCSVMHEALHNHYTQKSLSLSLG (SEQ ID NO: 471) and a light chain comprising the amino acid sequence of DIQMTQSPSSLSASVGDRVTITCKASDHILKFLTWYQQKPGKAPKLLIYGATSLETGV PSRFSGSGSGTDFTLTISSLQPEDFATYYCQMYWSTPFTFGGGTKVEIKRTVAAPSVFI FPPSDEQLKSGTASVVCLLNNFYPREAKVQWKVDNALQSGNSQESVTEQDSKDSTY SLSSTLTLSKADYEKHKVYACEVTHQGLSSPVTKSFNRGEC (SEQ ID NO: 472). In some embodiments, the IL23 inhibitor provided herein for the combination therapy comprises an anti-IL23 antibody or antigen-binding fragment thereof, wherein the antibody or antigen-binding fragment thereof comprises two light chains and two heavy chains, wherein each light chain comprises the amino acid sequence SEQ ID NO: 472 and each heavy chain comprises the amino acid sequence SEQ ID NO: 471.

In some embodiments, the IL23 inhibitor provided in this Section (Section 4.3.2(e)) for the combination therapy is in a pharmaceutical composition further comprising a one or more pharmaceutically acceptable carriers, diluents or excipients.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered for at least one induction dose of about 200 mg to about 1200 mg of the anti-IL23 antibody or antigen-binding fragment thereof and administered for at least one maintenance dose of about 100 mg to about 600 mg of the anti-IL23 antibody or antigen-binding fragment thereof. Further embodiments of the at least one induction dose and/or the at least one maintenance dose of the anti-IL23 antibody or antigen-binding fragment thereof for the combination therapy are described in the remainder of the paragraph. In some embodiments, induction dose comprises about 200 mg, about 300 mg, about 400 mg, about 500 mg, about 600 mg, about 700 mg, about 800 mg, about 900 mg, about 1000 mg, about 1100 mg or about 1200 mg of the anti-IL23 antibody or antigen-binding fragment thereof provided in this Section (Section 4.3.2(d)). In one embodiment, the at least one induction dose comprises about 900 mg of the anti-IL23 antibody or antigen-binding fragment thereof provided in this Section (Section 4.3.2(d)). In another embodiment, one, two, three or four induction doses are administered to the subject in the combination therapy. In a further embodiment, three induction doses are administered to the subject in the combination therapy at about 4-week intervals. In yet another embodiment, the at least one induction dose is administered by intravenous infusion. In one embodiment, if the subject has not achieved endoscopic response about 4 to about 12 weeks after the last induction dose is administered, at least one extended induction dose(s) of mirikizumab is administered to the subject, wherein the at least one maintenance dose(s) of mirikizumab is administered to the subject if the subject has achieved endoscopic response about 4 to about 12 weeks after the last extended induction dose is administered, and wherein endoscopic response is defined as a 50% reduction from baseline in SES-CD Score. In another embodiment, the at least one extended induction dose(s) are administered to the subject if the subject has not achieved endoscopic response about 4 weeks after the last induction dose is administered. In a further embodiment, multiple extended induction doses are administered at about 4 week intervals. In yet another embodiment, three extended induction doses are administered at about 4 week intervals. In one embodiment, the extended induction dose(s) comprise about 200 mg, about 600 mg, about 900 mg or about 1000 mg of mirikizumab. In another embodiment, the extended induction dose(s) comprise(s) about 900 mg of mirikizumab. In a further embodiment, the one, two or three extended induction dose(s) are administered by intravenous infusion. In yet another embodiment, the at least one maintenance dose comprises about 100 mg, about 150 mg, about 200 mg, about 250 mg, about 300 mg, about 350 mg, about 400 mg, about 500 mg or about 600 mg of mirikizumab. In one embodiment, the at least one maintenance dose comprises about 200 mg or about 300 mg of mirikizumab. In another embodiment, the at least one maintenance dose is administered 2-16 weeks after the last induction dose is administered. In a further embodiment, the at least one maintenance dose is administered about 2 weeks, about 3 weeks, about 4 weeks, about 5 weeks, about 6 weeks, about 7 weeks, about 8 weeks, about 12 weeks or about 16 weeks after the last induction dose is administered. In yet another embodiment, the at least one maintenance dose is administered about 4 weeks after the last induction dose is administered. In one embodiment, the at least one maintenance dose is administered about 8 weeks after the last induction dose is administered. In another embodiment, multiple maintenance doses are administered to a patient and wherein the first maintenance dose is administered 2 to 16 weeks after the last induction dose is administered. In a further embodiment, the first maintenance dose is administered about 2 weeks, about 3 weeks, about 4 weeks, about 5 weeks, about 6 weeks, about 7 weeks, about 8 weeks, about 12 weeks or about 16 weeks after the last induction dose is administered. In yet another embodiment, the first maintenance dose is administered about 4 weeks after the last induction dose is administered. In one embodiment, the first maintenance dose is administered about 8 weeks after the last induction dose is administered. In another embodiment, one or more further maintenance dose(s) are administered at about 4, about 8 or about 12 week interval(s) after administration of the first maintenance dose. In a further embodiment, one or more further maintenance dose(s) are administered at about 4 week interval(s) after administration of the first maintenance dose. In yet another embodiment, one or more further maintenance dose(s) are administered at about 8 week interval(s) after administration of the first maintenance dose. In one embodiment, the maintenance dose(s) are administered by subcutaneous injection. In another embodiment, (i) three induction doses of mirikizumab is administered to the subject by intravenous injection, wherein each induction dose comprises about 900 mg of mirikizumab, and (ii) maintenance dose(s) of mirikizumab is administered to the subject by subcutaneous injection at about 4 week or about 8 week intervals, wherein the first maintenance dose is administered about 4 weeks or about 8 weeks after the last induction dose is administered and wherein each maintenance dose comprises about 200 mg or about 300 mg of mirikizumab. In yet another embodiment, the three induction doses of mirikizumab are administered at about 4 week intervals and the first maintenance dose is administered about 4 weeks after the last induction dose is administered

In some embodiments of the combination therapy provided herein, the doses and/or the dosing regimens at which the IL23 inhibitor provided in this Section (Section 4.3.2(e)) is administered are the effective amount of the IL23 inhibitor of this Section (Section 4.3.2(e)) in the combination therapy.

In certain embodiments, the IL23 inhibitor comprises an anti-IL23 antibody or antigen binding fragment selected from those described in U.S. Pat. Nos. 9,023,358 and 9,688,753, and in the publication of WO/2020/219314 (PCT/US2020/028273). In one embodiment, the IL23 inhibitor comprises an anti-IL23 antibody or antigen binding fragment selected from those described in U.S. Pat. Nos. 9,023,358 and 9,688,753, and in the publication of WO/2020/219314 (PCT/US2020/028273), at a formulation and dose as described in U.S. Pat. Nos. 9,023,358 and 9,688,753, and in the publication of WO/2020/219314 (PCT/US2020/028273), the disclosures of all of which are hereby incorporated in their entireties by reference.

In certain embodiments, the IL23 inhibitor comprises anti-IL23 antibody or antigen binding fragment as described in clinical studies Sandborn W. J., Gastroenterology. 2020 February; 158 (3): 537-549.e10 and NCT03926130 (available at clinicaltrials.gov/ct2/show/NCT03926130). In one embodiment, the IL23 inhibitor comprises anti-IL23 antibody or antigen binding fragment as described in the clinical studies Sandborn W. J., Gastroenterology. 2020 February; 158 (3): 537-549.e10 and NCT03926130 (available at clinicaltrials.gov/ct2/show/NCT03926130), at a formulation and dose as described in the same clinical studies, the disclosures of all of which are hereby incorporated in their entireties by reference.

The disclosure further provides that the IL23 inhibitory effects of the anti-IL23 antibody or antigen binding fragment of this Section (Section 4.3.2(e)) have been validated in studies as further described in U.S. Pat. Nos. 9,023,358 and 9,688,753, and in the publication of WO/2020/219314 (PCT/US2020/028273), in clinical studies described in Sandborn W. J., Gastroenterology. 2020 February; 158 (3): 537-549.e10, and in the clinical study NCT03926130 (available at clinicaltrials.gov/ct2/show/NCT03926130), the disclosures of all of which are hereby incorporated in their entireties by reference.

In certain embodiments, the IL23 inhibitor of this Section (Section 4.3.2(e)) binds to the p19 subunit of the IL23. In certain embodiments, the IL23 inhibitor of this Section (Section 4.3.2(e)) binds to IL23 but does not bind to IL12.

Without being bound by the theory, the disclosure provides the disclosure provides that mirikizumab binds to the p19 subunit of IL23 and inhibit IL23 function (antagonist of IL23).

### (f) Tildrakizumab

In some embodiments, the IL23 inhibitor provided herein for the combination therapy comprises tildrakizumab. In some embodiments, the IL23 inhibitor provided herein for the combination therapy comprises an anti-IL23 antibody or antigen-binding fragment thereof, wherein the antibody or antigen-binding fragment thereof comprises: (a) an antibody light chain variable region comprising CDRL1, CDRL2 and CDRL3, wherein CDRL1 comprises the sequence of RTSENIYSYLA (SEQ ID NO: 473), CDRL2 comprises the sequence of NAKTLAE (SEQ ID NO: 474), and CDRL3 comprises the sequence of QHHYGIPFT (SEQ ID NO: 475); and (b) an antibody heavy chain variable region comprising CDRH1, CDRH2 and CDRH3, wherein CDRH1 comprises the sequence of GYIFITYWMT (SEQ ID NO: 476), CDRH2 comprises the sequence of QIFPASGSADYNEKFEG (SEQ ID NO: 477), and CDRH3 comprises the sequence of GGGGFAY (SEQ ID NO: 478) (such antibodies and antigen-binding fragments, tildrakizumab). In some embodiments, the IL23 inhibitor provided herein for the combination therapy comprises an anti-IL23 antibody or antigen-binding fragment thereof, wherein the antibody or antigen-binding fragment thereof comprises a heavy chain variable region comprising the amino acid sequence of residues 1-116 of SEQ ID NO: 480 and a light chain variable region comprising the amino acid sequence of residues 1-108 of SEQ ID NO: 479.

In some embodiments, the IL23 inhibitor provided herein for the combination therapy comprises an anti-IL23 antibody or antigen-binding fragment thereof, wherein the antibody comprises a heavy chain comprising the amino acid sequence of QVQLVQSGAEVKKPGASVKVSCKASGYIFITYWMTWVRQAPGQGLEWMGQIFPAS GSADYNEKFEGRVTMTTDTSTSTAYMELRSLRSDDTAVYYCARGGGGFAYWGQGT LVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTC PPCPAPELLGGPSVFLFPPKPKDTLMISRTPEVTCVVVDVSHEDPEVKFNWYVDGVE VHNAKTKPREEQYNSTYRVVSVLTVLHQDWLNGKEYKCKVSNKALPAPIEKTISKA KGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 480) and a light chain comprising the amino acid sequence of DIQMTQSPSSLSASVGDRVTITCRTSENIYSYLAWYQQKPGKAPKLLIYNAKTLAEGV PSRFSGSGSGTDFTLTISSLQPEDFATYYCQHHYGIPFTFGQGTKVEIKRTVAAPSVFIF PPSDEQLKSGTASVVCLLNNFYPREAKVQWKVDNALQSGNSQESVTEQDSKDSTYS LSSTLTLSKADYEKHKVYACEVTHQGLSSPVTKSFNRGEC (SEQ ID NO: 479).

In some embodiments, the IL23 inhibitor provided herein for the combination therapy comprises an anti-IL23 antibody or antigen-binding fragment thereof, wherein the antibody or antigen-binding fragment thereof binds to human IL-23 at an epitope comprising residues 20-30 and 82-110 of RAVPGGSSPAWTQCQQLSQKLCTLAWSAHPLVGHMDLREEGDEETTNDVPHIQCGD GCDPQGLRDNSQFCLQRIHQGLIFYEKLLGSDIFTGEPSLLPDSPVGQLHASLLGLSQL LQPEGHHWETQQIPSLSPSQPWQRLLLRFKILRSLQAFVAVAARVFAHGAATLSP (SEQ ID NO: 481), and wherein the antibody or antigen-binding fragment thereof comprises: (a) an antibody light chain variable region comprising CDRL1, CDRL2 and CDRL3, wherein CDRL1 comprises the sequence of SEQ ID NO: 473, CDRL2 comprises the sequence of SEQ ID NO: 474, and CDRL3 comprises the sequence of SEQ ID NO: 475; and (b) an antibody heavy chain variable region comprising CDRH1, CDRH2 and CDRH3, wherein CDRH1 comprises the sequence of SEQ ID NO: 476, CDRH2 comprises a sequence selected from the group consisting of SEQ ID NO: 477, and CDRH3 comprises the sequence of SEQ ID NO: 478. In some embodiments, the IL23 inhibitor provided herein for the combination therapy comprises an anti-IL23 antibody or antigen-binding fragment thereof, wherein the antibody or antigen-binding fragment thereof binds to human IL-23 at an epitope comprising residues K20, T23, W26, S27, P30, E82, S95, L96, L97, P98, D99, P101, G103, Q104, H106, A107, and L110 of SEQ ID NO: 481. In some embodiments, the antibody or antigen-binding fragment thereof further binds to residues L24, L85, T91, S100, and V102 of SEQ ID NO: 481.

In some embodiments, the IL23 inhibitor provided in this Section (Section 4.3.2(f)) for the combination therapy is in a pharmaceutical composition further comprising a one or more pharmaceutically acceptable carriers or diluent.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered at Weeks 0, 4, and every twelve weeks thereafter, wherein each administration comprising administering 100 mg of the anti-IL23 antibody or antigen-binding fragment thereof. In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered at Weeks 0, 4, and every twelve weeks thereafter up to 52 weeks, wherein each administration comprising administering 100 mg of the anti-IL23 antibody or antigen-binding fragment thereof.

In some embodiments of the combination therapy provided herein, the doses and/or the dosing regimens at which the IL23 inhibitor provided in this Section (Section 4.3.2(e)) is administered are the effective amount of the IL23 inhibitor of this Section (Section 4.3.2(e)) in the combination therapy.

In certain embodiments, the IL23 inhibitor comprises an anti-IL23 antibody or antigen binding fragment thereof selected from those described in U.S. Pat. Nos. 8,404,813, 8,293,883, and 9,809,648. In one embodiment, the IL23 inhibitor comprises an amiselimod or a derivative of amiselimod selected from those described in U.S. Pat. Nos. 8,404,813, 8,293,883, and 9,809,648, at a formulation and dose as described in U.S. Pat. Nos. 8,404,813, 8,293,883, and 9,809,648, the disclosures of all of which are hereby incorporated in their entireties by reference.

In certain embodiments, the IL23 inhibitor comprises tildrakizumab as described in the US FDA approved label for ILUMYA (revised March 2018, available at accessdata.fda.gov/drugsatfda_docs/label/2018/761067s000lbl.pdf). In one embodiment, the IL23 inhibitor comprises tildrakizumab as described in the US FDA approved label for ILUMYA (revised March 2018, available at accessdata.fda.gov/drugsatfda_docs/label/2018/761067s000lbl.pdf), at a formulation and dose as described in the same US FDA approved label, the disclosures of all of which are hereby incorporated in their entireties by reference.

The disclosure further provides that the IL23 inhibitory effects of the anti-IL23 antibody or antigen binding fragment thereof of this Section (Section 4.3.2(f)) have been validated in studies as further described in U.S. Pat. Nos. 8,404,813, 8,293,883, and 9,809,648, and in clinical studies described in Gooderham M. et al., J Eur Acad Dermatol Venereol. 2019 October; 33 (10): e350-e352, Reich K et al., Lancet. 2017 Jul. 15; 390 (10091): 276-288, and in the US FDA approved label for ILUMYA (revised March 2018, available at accessdata.fda.gov/drugsatfda_docs/label/2018/761067s000lbl.pdf), the disclosures of all of which are hereby incorporated in their entireties by reference.

In certain embodiments, the IL23 inhibitor of this Section (Section 4.3.2(f)) binds to the p19 subunit of the IL23. In certain embodiments, the IL23 inhibitor of this Section (Section 4.3.2(f)) binds to IL23 but does not bind to IL12.

Without being bound by the theory, the disclosure provides that tildrakizumab binds to the p19 subunit of IL23 and inhibit IL23 function (antagonist of IL23).

### (g) Briakinumab

In some embodiments, the IL23 inhibitor provided herein for the combination therapy comprises briakinumab. In some embodiments, the IL23 inhibitor provided herein for the combination therapy comprises an anti-IL23 antibody or antigen-binding fragment thereof, wherein the antibody or antigen-binding fragment thereof comprises: (a) an antibody light chain variable region comprising CDRL1, CDRL2 and CDRL3, wherein CDRL1 comprises the sequence of SGSRSNIGSNTVK (SEQ ID NO: 482), CDRL2 comprises the sequence of YNDQRPS (SEQ ID NO: 483), and CDRL3 comprises the sequence of QSYDRYTHPALL (SEQ ID NO: 484); and (b) an antibody heavy chain variable region comprising CDRH1, CDRH2 and CDRH3, wherein CDRH1 comprises the sequence of FTFSSYGMH (SEQ ID NO: 485), CDRH2 comprises the sequence of FIRYDGSNKYYADSVKG (SEQ ID NO: 486), and CDRH3 comprises the sequence of HGSHDN (SEQ ID NO: 487) (such antibodies and antigen-binding fragments, briakinumab).

In some embodiments, the IL23 inhibitor provided herein for the combination therapy comprises an anti-IL23 antibody or antigen-binding fragment thereof, wherein the antibody or antigen-binding fragment thereof comprises a heavy chain variable region comprising the amino acid sequence of QVQLVESGGGVVQPGRSLRLSCAASGFTFSSYGMHWVRQAPGKGLEWVAFIRYDG SNKYYADSVKGRFTISRDNSKNTLYLQMNSLRAEDTAVYYCKTHGSHDNWGQGTM VTVSS (SEQ ID NO: 488) and a light chain variable region comprising the amino acid sequence of QSVLTQPPSVSGAPGQRVTISCSGSRSNIGSNTVKWYQQLPGTAPKLLIYYNDQRPSG VPDRFSGSKSGTSASLAITGLQAEDEADYYCQSYDRYTHPALLFGTGTKVTVLG (SEQ ID NO: 489).

In some embodiments, the IL23 inhibitor provided herein for the combination therapy comprises an anti-IL23 antibody, wherein the antibody further comprises a heavy chain constant region selected from the group consisting of IgG1, IgG2, IgG3, IgG4, IgM, IgA and IgE constant regions. In some embodiments, the IL23 inhibitor provided herein for the combination therapy comprises an anti-IL23 antibody, wherein the antibody further comprises a IgG1 heavy chain constant region.

In certain embodiments, the IL23 inhibitor provided herein for the combination therapy comprises an anti-IL23 antibody or antigen-binding fragment thereof, wherein the antibody or antigen-binding fragment thereof binds to human IL-12 and dissociates from human IL-12 with a K_{D} of 1×10⁻¹⁰ M or less and a k_{off} rate constant of 1×10⁻³ s⁻¹ or less, as determined by surface plasmon resonance. In some embodiments, the antibody or antigen-binding fragment thereof disassociates from human IL-12 with a k_{off} rate constant of 1×10⁻⁴ s⁻¹ or less. In some embodiments, the antibody or antigen-binding fragment thereof disassociates from human IL-12 with a k_{off} rate constant of 1×10⁻⁵ s⁻¹ or less. In some embodiments, the antibody or antigen-binding fragment thereof binds to human IL-12 and disassociates from human IL-12 with a K_{D} of 1.34×10⁻¹⁰ M or less. In some embodiments, the antibody or antigen-binding fragment thereof binds to human IL-12 and disassociates from human IL-12 with a K_{D} of 1.34×10⁻¹¹ M or less. In some embodiments, the antibody or antigen-binding fragment thereof is a neutralizing antibody against IL23 and/or IL12. In some embodiments, the antibody or antigen-binding fragment thereof inhibits phytohemagglutinin blast proliferation in an in vitro PHA assay with an IC₅₀ of 1×10⁻⁹M or less. In some embodiments, the antibody or antigen-binding fragment thereof inhibits phytohemagglutinin blast proliferation in an in vitro phytohemagglutinin blast proliferation assay (PHA assay) with an IC₅₀ of 1×10⁻¹⁰M or less. In some embodiments, the antibody or antigen-binding fragment thereof inhibits phytohemagglutinin blast proliferation in an in vitro PHA assay with an IC₅₀ of 1×10⁻¹¹M or less. In some embodiments, the antibody or antigen-binding fragment thereof inhibits phytohemagglutinin blast proliferation in an in vitro PHA assay with an IC₅₀ of 1×10⁻¹¹M or less. In some embodiments, the antibody or antigen-binding fragment thereof inhibits phytohemagglutinin blast proliferation in an in vitro PHA assay with an IC₅₀ of 1×10⁻⁷M or less. In some embodiments, the antibody or antigen-binding fragment thereof inhibits phytohemagglutinin blast proliferation in an in vitro PHA assay with an IC₅₀ of 1×10⁻⁸M or less. In some embodiments, the antibody or antigen-binding fragment thereof inhibits phytohemagglutinin blast proliferation in an in vitro PHA assay with an IC₅₀ of 1×10⁻¹¹M or less. In some embodiments, the antibody or antigen-binding fragment thereof inhibits human IFNγ production with an IC₅₀ of 1×10⁻¹⁰ M or less. In some embodiments, the antibody or antigen-binding fragment thereof inhibits human IFNγ production with an IC₅₀ of 1×10⁻¹¹ M or less. In some embodiments, the antibody or antigen-binding fragment thereof inhibits human IFNγ production with an IC₅₀ of 1×10⁻¹² M or less. In some embodiments, the antibody or antigen-binding fragment thereof binds to human IL-12 and disassociates from human IL-12 with a K_{off} rate constant of 1×10⁻²s 1 or less, as determined by surface plasmon resonance. In some embodiments, the antibody or antigen-binding fragment thereof binds to human IL-12 and disassociates from human IL-12 with a K_{off} rate constant of 1×10⁻³s 1 or less, as determined by surface plasmon resonance. In some embodiments, the antibody or antigen-binding fragment thereof binds to human IL-12 and disassociates from human IL-12 with a K_{off} rate constant of 1×10⁻⁴s 1 or less, as determined by surface plasmon resonance. In some embodiments, the antibody or antigen-binding fragment thereof binds to human IL-12 and disassociates from human IL-12 with a K_{off} rate constant of 1×10⁻⁵s 1 or less, as determined by surface plasmon resonance.

In some embodiments, the IL23 inhibitor provided in this Section (Section 4.3.2(g)) for the combination therapy is in a pharmaceutical composition further comprising a one or more pharmaceutically acceptable carriers or diluent.

In some embodiments, the antibody or antigen-binding fragment thereof provided in this Section (Section 4.3.2(g)) binds to an epitope on the p40 subunit of human IL12/IL23.

In some embodiments of the combination therapy provided herein, the IL23 inhibitor comprises the anti-IL23 antibody or antigen-binding fragment thereof provided in this Section (Section 4.3.2(d)) and the anti-IL23 antibody or antigen-binding fragment thereof is administered (i) for a first dose amount of 180 mg to 220 mg of the antibody or antigen-binding domain thereof, at week 0, and for the same first dose amount of the antibody or antigen-binding domain thereof at week 4, and (ii) for a second dose amount of 80 mg to 120 mg of the antibody or antigen-binding domain thereof every 4 weeks thereafter. In some embodiment, the first dose amount of the antibody or antigen-binding domain thereof is 200 mg. In some embodiment, the second dose amount of the antibody or antigen-binding domain thereof is 100 mg. In some embodiment, the first dose amount of the antibody or antigen-binding domain thereof is 200 mg and the second dose amount of the antibody or antigen-binding domain thereof is 100 mg.

In some embodiments of the combination therapy provided herein, the doses and/or the dosing regimens at which the IL23 inhibitor provided in this Section (Section 4.3.2(g)) is administered are the effective amount of the IL23 inhibitor of this Section (Section 4.3.2(g)) in the combination therapy.

In certain embodiments, the IL23 inhibitor comprises an anti-IL23 antibody or antigen binding fragment thereof selected from those described in U.S. Pat. Nos. 6,914,128, 7,504,485, 8,865,174, 9,035,030, and 8,557,239. In one embodiment, the IL23 inhibitor comprises an anti-IL23 antibody or antigen binding fragment thereof selected from those described in U.S. Pat. Nos. 6,914,128, 7,504,485, 8,865,174, 9,035,030, and 8,557,239, at a formulation and dose as described in U.S. Pat. Nos. 6,914,128, 7,504,485, 8,865,174, 9,035,030, and 8,557,239, the disclosures of all of which are hereby incorporated in their entireties by reference.

In certain embodiments, the IL23 inhibitor comprises an anti-IL23 antibody or antigen binding fragment thereof as described in Grodon K. B. et al., J Invest Dermatol. 2012 February; 132 (2): 304-14, and Remo Panaccione et al., Inflamm Bowel Dis. 2015 June; 21 (6): 1329-40. In one embodiment, the IL23 inhibitor comprises an anti-IL23 antibody or antigen binding fragment thereof as described in Grodon K. B. et al., J Invest Dermatol. 2012 February; 132 (2): 304-14, and Remo Panaccione et al., Inflamm Bowel Dis. 2015 June; 21 (6): 1329-40, at a formulation and dose as described in the same publications, the disclosures of all of which are hereby incorporated in their entireties by reference.

The disclosure further provides that the IL23 inhibitory effects of the antibody or antigen-binding fragments of this Section (Section 4.3.2(g)) have been validated in studies as further described in U.S. Pat. Nos. 6,914,128, 7,504,485, 8,865,174, 9,035,030, and 8,557,239, and in clinical studies described in Grodon K. B. et al., J Invest Dermatol. 2012 February; 132 (2): 304-14, and Remo Panaccione et al., Inflamm Bowel Dis. 2015 June; 21 (6): 1329-40, the disclosures of all of which are hereby incorporated in their entireties by reference.

Without being bound by the theory, the disclosure provides that briakinumab is an antibody or antigen binding fragment to the p40 subunit of interleukin-12 (p35/p40, abbreviated as IL12 or IL-12) and interleukin-23 (p19/p40, abbreviated as IL23 or IL-23) and thus binds to both IL12 and IL23.

### (h) Anti-IL23 Antibodies or Antigen-Binding Fragments in General and Additional Anti-IL23 Antibodies or Antigen-Binding Fragments Thereof

In some embodiments, the IL23 inhibitor provided herein for the combination therapy comprises anti-IL23 antibodies or antigen-binding fragments thereof. In certain embodiments, the IL23 inhibitor provided herein for the combination therapy comprises anti-IL23 antibodies or antigen-binding fragments thereof that are specific for IL23. In one embodiment, the IL23 inhibitor provided herein for the combination therapy comprises anti-IL23 antibodies or antigen-binding fragments thereof, wherein the anti-IL23 antibodies or antigen-binding fragments thereof binds to the p19 subunit of IL23 and does not bind to the p40 subunit of IL23. In one embodiment, the IL23 inhibitor provided herein for the combination therapy comprises anti-IL23 antibodies or antigen-binding fragments thereof, wherein the anti-1123 antibodies or antigen-binding fragments thereof binds to the p19 subunit of IL23 and does not bind to the p40 subunit of IL23. In one embodiment, the IL23 inhibitor provided herein for the combination therapy comprises anti-IL23 antibodies or antigen-binding fragments thereof, wherein the anti-1123 antibodies or antigen-binding fragments thereof binds to the p19 subunit of IL23 and binds to the p40 subunit of IL23. In one embodiment, the IL23 inhibitor provided herein for the combination therapy comprises anti-IL23 antibodies or antigen-binding fragments thereof, wherein the anti-IL23 antibodies or antigen-binding fragments thereof inhibits binding of IL23 to IL23 receptor (a heterodimer between IL-12RB1 and IL-23R). In certain embodiments, the anti-IL23 antibodies or antigen-binding fragments thereof of this paragraph can be generated and prepared as described in Section 4.4 and validated with assays described in Section 4.3.3.

### 6.3.3 Assays

An exemplary screening paradigm for identification of antibody variants that express well in mammalian cells and preserve TL1A binding activity while minimizing the propensity of the antibody to aggregate comprises a five-step process. This screen was performed as detailed in the examples. Briefly, (1) variants were cloned and transiently expressed as intact Ig in 293 cells using small-scale (3 mL, 6-well culture plates) transfections, (2) the expression level of the antibody was assessed in the culture supernatant 96-120 hours after transfection using an antibody quantitation ELISA, (3) the binding of the supernatant antibody variants to human TL1A was assessed by ELISA, (4) the antibody was purified in a single step using Protein A and (5) the material was analyzed by analytical SEC to assess monomer/aggregate content. This approach enabled identification of variants that expressed well, preserved binding to TL1A, and displayed high monomer content.

Further provided herein are methods for analyzing antibody solubility based on percentage of monomeric fraction. For example, as described in Example 2.

Further provided herein are assays for quantifying antibody expression. For example, as described in Example 2.

Further provided herein are assays for quantifying immunogenicity of an antibody.

The inhibitors, including TL1A inhibitors, IL23 inhibitors and/or antibodies described herein can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are provided in for e.g., Ausubel et al., eds, 1994, Current Protocols in Molecular Biology, Vol. 1, John Wiley & Sons, Inc., New York.

### 6.4 Methods of Generating Antibodies

In various embodiments, monoclonal antibodies are prepared using methods known in the art, such as, but not limited to the hybridoma method, where a host animal is immunized to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid by techniques known in the art, when propagated either in vitro or in vivo.

In some embodiments, monoclonal antibodies are made using recombinant DNA methods. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, a chimeric antibody, a molecule in which different portions are derived from different animal species, such as those having a variable region derived from a murine monoclonal antibody and a human immunoglobulin constant region (e.g., humanized antibodies) can be generated.

In some embodiments, the anti-TL1A or the anti-IL23 monoclonal antibody is a humanized antibody, to reduce antigenicity and HAMA (human anti-mouse antibody) responses when administered to a human subject. Humanized antibodies can be produced using various techniques known in the art. For example, an antibody is humanized by (1) determining the nucleotide and predicted amino acid sequence of the starting antibody light and heavy variable domains; (2) designing the humanized antibody, e.g., deciding which antibody framework region to use during the humanizing process; (3) the actual humanizing methodologies/techniques; and (4) the transfection and expression of the humanized antibody. In various embodiments, a humanized antibody can be further optimized to decrease potential immunogenicity, while maintaining functional activity, for therapy in humans.

Humanized antibodies can also be made in transgenic mice containing human immunoglobulin loci that are capable, upon immunization, of producing the full repertoire of human antibodies in the absence of endogenous immunoglobulin production. A humanized antibody may also be obtained by a genetic engineering approach that enables production of affinity-matured human-like polyclonal antibodies in large animals.

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications should therefore be restricted to a minimum and closely watched for changes in the specificity and affinity of the antibody. On the other hand, framework residues in theory can be derived from any human variable region. A human framework sequences should be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g., mutated or affinity matured) sequences. Genetic engineering techniques well known to those in the art, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR.

In certain embodiments, the anti-TL1A or the anti-IL23 antibody is a human antibody. Human antibodies can be directly prepared using various techniques known in the art. Immortalized human B lymphocytes immunized in vitro or isolated from an immunized individual that produce an antibody directed against a target antigen can be generated.

Chimeric, humanized and human antibodies may be produced by recombinant expression. Recombinant polynucleotide constructs typically include an expression control sequence operably linked to the coding sequences of antibody chains, including naturally associated or heterologous promoter regions. In certain embodiments, it may be desirable to generate amino acid sequence variants of these humanized antibodies, particularly where these improve the binding affinity or other biological properties of the antibody.

In certain embodiments, an antibody fragment is used to treat and/or ameliorate IBD. Various techniques are known for the production of antibody fragments. Generally, these fragments are derived via proteolytic digestion of intact antibodies (for example Morimoto et al., 1993, Journal of Biochemical and Biophysical Methods 24:107-117; Brennan et al., 1985, Science, 229:81). Fab, Fv, and scFv antibody fragments can all be expressed in and secreted from E. coli or other host cells, thus allowing the production of large amounts of these fragments. Other techniques for the production of antibody fragments will be apparent to the skilled practitioner.

According to the present disclosure, techniques can be adapted for the production of single-chain antibodies specific to TL1A or IL23. In addition, methods can be adapted for the construction of Fab expression libraries to allow rapid and effective identification of monoclonal Fab fragments with the desired specificity for TL1A or IL23, or derivatives, fragments, analogs or homologs thereof. Antibody fragments may be produced by techniques in the art including, but not limited to: (a) a F(ab′)₂ fragment produced by pepsin digestion of an antibody molecule; (b) a Fab fragment generated by reducing the disulfide bridges of an F(ab′)₂ fragment, (c) a Fab fragment generated by the treatment of the antibody molecule with papain and a reducing agent, and (d) Fv fragments.

Also provided herein are modified antibodies comprising any type of variable region that provides for the association of the antibody with TL1A or IL23. Those skilled in the art will appreciate that the modified antibodies may comprise antibodies (e.g., full-length antibodies or immunoreactive fragments thereof) in which at least a fraction of one or more of the constant region domains has been deleted or otherwise altered so as to provide desired biochemical characteristics such as decreasing TL1A or IL23. In certain embodiments, the variable regions in both the heavy and light chains are altered by at least partial replacement of one or more CDRs and, if necessary, by partial framework region replacement and sequence changing. In some embodiments, the replaced CDRs may be derived from an antibody of the same class, subclass, from an antibody of a different class, for instance, from an antibody from a different species and/or a combination thereof. In some embodiments, the constant region of the modified antibodies will comprise a human constant region. Modifications to the constant region compatible with this disclosure comprise additions, deletions or substitutions of one or more amino acids in one or more domains.

In various embodiments, the expression of an antibody or antigen-binding fragment thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody or antigen-fragment thereof as described herein may be transfected into the host.

In some embodiments, the expression vectors are transfected into the recipient cell line for the production of the chimeric, humanized, or composite human antibodies described herein. In various embodiments, mammalian cells can be useful as hosts for the production of antibody proteins, which can include, but are not limited to cells of fibroblast origin, such as Vero (ATCC CRL 81) or CHO-K1 (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO—S and DG44 cells; PER.C6™ cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying a chimeric, humanized, or composite human antibody construct, antibody or antigen-binding fragment thereof as described herein can be introduced into an appropriate host cell by any of a variety of suitable means, depending on the type of cellular host including, but not limited to transformation, transfection, lipofection, conjugation, electroporation, direct microinjection, and microprojectile bombardment, as known to one of ordinary skill in the art. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody molecules or peptides described herein. In various other embodiments, bacterial strains can also be utilized as hosts for the production of the antibody molecules or peptides described herein. Examples of bacterial strains include, but are not limited to E. coli, Bacillus species, enterobacteria, and various Pseudomonas species.

In some embodiments, one or more antibodies or antigen-binding fragments thereof as described herein can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982)).

Once expressed in the host, the whole antibodies, antibody-fragments (e.g., individual light and heavy chains), or other immunoglobulin forms of the present disclosure can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, etc. See generally, Vols. I & II Immunol. Meth. (Lefkovits & Pernis, eds., Acad. Press, NY, 1979 and 1981).

Various embodiments provide for a genetic construct comprising a nucleic acid encoding an anti-TL1A or anti-IL23 antibody or fragment provided herein. Genetic constructs of the antibody can be in the form of expression cassettes, which can be suitable for expression of the encoded anti-TL1A or anti-IL23 antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by methods known in the art. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3T3, Chinese hamster ovary (CHO), HeLa and BHK cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine (SEQ ID NO: 391), maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography. Recombinant protein produced in bacterial culture can be isolated.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Particular conservative substitutions include, for example; Ala into Gly or into Ser; Arg into Lys; Asn into Gin or into His; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding fragment thereof described herein can be a variant of a sequence described herein, e.g., a conservative substitution variant of an antibody polypeptide. In some embodiments, the variant is a conservatively modified variant. A variant may refer to a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g., antigen-specific binding activity for the relevant target polypeptide.

Alterations of the native amino acid sequence can be accomplished by any of a number of techniques known to one of skill in the art. Mutations can be introduced at particular loci or by oligonucleotide-directed site-specific mutagenesis procedures. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981).

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, NY, 1982 and 1989), and can be used to construct nucleic acid sequences which encode a monoclonal antibody molecule or antigen-binding region.

In some embodiments, a nucleic acid encoding an antibody or antigen-binding fragment thereof as described herein is comprised by a vector. In some of the aspects described herein, a nucleic acid sequence encoding an antibody or antigen-binding fragment thereof as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding domain thereof as described herein in place of non-essential viral genes. The vector and/or particle may be utilized for the purpose of transferring any nucleic acids into cells either in vitro or in vivo. Numerous forms of viral vectors are known in the art.

By “recombinant vector,” it is meant that the vector includes a heterologous nucleic acid sequence, or “transgene” that is capable of expression in vivo.

### 6.5 Pharmaceutical Compositions

In one aspect, TL1A inhibitors and/or IL23 inhibitors provided herein are formulated into pharmaceutical compositions that are useful in a variety of applications including, but not limited to, therapeutic methods, such as the treatment of IBD. The methods of use may be in vitro, ex vivo, or in vivo methods. In certain embodiments, the disease treated with the combination therapy of a TL1A inhibitor and an IL23 inhibitor is IBD, CD, UC and/or MR-UC.

In various embodiments, the pharmaceutical compositions are formulated for delivery via any route of administration. “Route of administration” includes any administration pathway known in the art, including but not limited to intravenous, subcutaneous, aerosol, nasal, oral, transmucosal, transdermal and parenteral. In example embodiments, the route of administration is subcutaneous.

The pharmaceutical compositions may contain any pharmaceutically acceptable carrier. “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that does not carry a risk of toxicity, irritation, allergic response, immunogenicity, or any other complication that excessively outweighs its therapeutic benefits.

In various embodiments, provided are pharmaceutical compositions including a pharmaceutically acceptable excipient along with a therapeutically effective amount of a TL1A inhibitor (such as an anti-TL1A antibody or antigen-binding fragment there of) or an IL23 inhibitor (such as an anti-IL23 antibody or antigen-binding fragment there of). “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in therapeutic methods described herein. Such excipients may be solid, liquid, semisolid, or, in the case of an aerosol composition, gaseous. Suitable excipients may be selected for different routes of administration (e.g., subcutaneous, intravenous, oral). Non-limiting examples include, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain auxiliary substances such as wetting or emulsifying agents, pH buffering agents and the like which enhance or maintain the effectiveness of the active ingredient. Therapeutic compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of the TL1A inhibitor or the IL23 inhibitor used that will be effective in the treatment of a particular disorder or condition will depend on the nature of the disorder or condition and can be determined by one of skill in the art with standard clinical techniques.

### Non-Limiting Example Compositions

In certain embodiments, provided herein are pharmaceutical compositions comprising a TL1A inhibitor and/or an IL23 inhibitor formulated for intravenous administration.

In certain embodiments, provided herein are pharmaceutical compositions comprising a TL1A inhibitor and/or an IL23 inhibitor formulated for subcutaneous administration.

In certain embodiments, provided herein are pharmaceutical compositions comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about or greater than about 150 mg/mL. In some embodiments, the concentration is up to about 300 mg/mL. In some embodiments, the concentration is about or greater than about 155, 160, 165, 170, 175, 180, 185, 190, 195, or 200 mg/mL. In some embodiments, the concentration is about 150 mg/mL to about 300 mg/mL, about 150 mg/mL to about 250 mg/mL, about 150 mg/mL to about 225 mg/mL, about 150 mg/mL to about 220 mg/mL, about 150 mg/mL to about 210 mg/mL, about 150 mg/mL to about 200 mg/mL, about 150 mg/mL to about 190 mg/mL, about 150 mg/mL to about 180 mg/mL, about 160 mg/mL to about 300 mg/mL, about 160 mg/mL to about 250 mg/mL, about 160 mg/mL to about 225 mg/mL, about 160 mg/mL to about 220 mg/mL, about 160 mg/mL to about 210 mg/mL, about 160 mg/mL to about 200 mg/mL, about 160 mg/mL to about 190 mg/mL, about 160 mg/mL to about 180 mg/mL, about 170 mg/mL to about 300 mg/mL, about 170 mg/mL to about 250 mg/mL, about 170 mg/mL to about 225 mg/mL, about 170 mg/mL to about 220 mg/mL, about 170 mg/mL to about 210 mg/mL, about 170 mg/mL to about 200 mg/mL, about 170 mg/mL to about 190 mg/mL, or about 170 mg/mL to about 180 mg/mL. In some embodiments, about 150 mg to about 1,000 mg of the TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) is present in the composition. For instance, about 150 mg to about 2000 mg, about 150 mg to about 1750 mg, about 150 mg to about 1500 mg, about 150 mg to about 1250 mg, about 150 mg to about 1000 mg, about 150 mg to about 750 mg, about 150 to about 500 mg, about 150 to about 300 mg, about 150 to about 200 mg, or about 150, 155, 160, 165, 170, 175, 180, 185, 190, 195, 200, 205, 210, 215, 220, 225 mg, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of the TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) can be present in the composition.

Additionally, in some embodiments of the composition provided herein, the composition comprises a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration greater than about 50 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration greater than about 55 mg/mL, greater than about 60 mg/mL, greater than about 65 mg/mL, greater than about 70 mg/mL, greater than about 75 mg/mL, greater than about 80 mg/mL, greater than about 85 mg/mL, greater than about 90 mg/mL, greater than about 95 mg/mL, greater than about 100 mg/mL, greater than about 105 mg/mL, greater than about 110 mg/mL, greater than about 115 mg/mL, greater than about 120 mg/mL, greater than about 125 mg/mL, greater than about 130 mg/mL, greater than about 135 mg/mL, greater than about 140 mg/mL, or greater than about 145 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, or about 145 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 250 mg/mL, about 55 mg/mL to about 250 mg/mL, about 60 mg/mL to about 250 mg/mL, about 65 mg/mL to about 250 mg/mL, about 70 mg/mL to about 250 mg/mL, about 75 mg/mL to about 250 mg/mL, about 80 mg/mL to about 250 mg/mL, about 85 mg/mL to about 250 mg/mL, about 90 mg/mL to about 250 mg/mL, about 95 mg/mL to about 250 mg/mL, about 100 mg/mL to about 250 mg/mL, about 105 mg/mL to about 250 mg/mL, about 110 mg/mL to about 250 mg/mL, about 115 mg/mL to about 250 mg/mL, about 120 mg/mL to about 250 mg/mL, about 125 mg/mL to about 250 mg/mL, about 130 mg/mL to about 250 mg/mL, about 135 mg/mL to about 250 mg/mL, about 140 mg/mL to about 250 mg/mL, about 145 mg/mL to about 250 mg/mL, about 150 mg/mL to about 250 mg/mL, about 155 mg/mL to about 250 mg/mL, about 160 mg/mL to about 250 mg/mL, about 165 mg/mL to about 250 mg/mL, about 170 mg/mL to about 250 mg/mL, about 175 mg/mL to about 250 mg/mL, about 180 mg/mL to about 250 mg/mL, about 185 mg/mL to about 250 mg/mL, about 190 mg/mL to about 250 mg/mL, about 195 mg/mL to about 250 mg/mL, about 200 mg/mL to about 250 mg/mL, about 205 mg/mL to about 250 mg/mL, about 210 mg/mL to about 250 mg/mL, about 215 mg/mL to about 250 mg/mL, about 220 mg/mL to about 250 mg/mL, about 225 mg/mL to about 250 mg/mL, about 230 mg/mL to about 250 mg/mL, about 235 mg/mL to about 250 mg/mL, about 240 mg/mL to about 250 mg/mL, about 245 mg/mL to about 250 mg/mL, about 50 mg/mL to about 240 mg/mL, about 55 mg/mL to about 240 mg/mL, about 60 mg/mL to about 240 mg/mL, about 65 mg/mL to about 240 mg/mL, about 70 mg/mL to about 240 mg/mL, about 75 mg/mL to about 240 mg/mL, about 80 mg/mL to about 240 mg/mL, about 85 mg/mL to about 240 mg/mL, about 90 mg/mL to about 240 mg/mL, about 95 mg/mL to about 240 mg/mL, about 100 mg/mL to about 240 mg/mL, about 105 mg/mL to about 240 mg/mL, about 110 mg/mL to about 240 mg/mL, about 115 mg/mL to about 240 mg/mL, about 120 mg/mL to about 240 mg/mL, about 125 mg/mL to about 240 mg/mL, about 130 mg/mL to about 240 mg/mL, about 135 mg/mL to about 240 mg/mL, about 140 mg/mL to about 240 mg/mL, about 145 mg/mL to about 240 mg/mL, about 150 mg/mL to about 240 mg/mL, about 155 mg/mL to about 240 mg/mL, about 160 mg/mL to about 240 mg/mL, about 165 mg/mL to about 240 mg/mL, about 170 mg/mL to about 240 mg/mL, about 175 mg/mL to about 240 mg/mL, about 180 mg/mL to about 240 mg/mL, about 185 mg/mL to about 240 mg/mL, about 190 mg/mL to about 240 mg/mL, about 195 mg/mL to about 240 mg/mL, about 200 mg/mL to about 240 mg/mL, about 205 mg/mL to about 240 mg/mL, about 210 mg/mL to about 240 mg/mL, about 215 mg/mL to about 240 mg/mL, about 220 mg/mL to about 240 mg/mL, about 225 mg/mL to about 240 mg/mL, about 230 mg/mL to about 240 mg/mL, about 235 mg/mL to about 240 mg/mL, about 50 mg/mL to about 230 mg/mL, about 55 mg/mL to about 230 mg/mL, about 60 mg/mL to about 230 mg/mL, about 65 mg/mL to about 230 mg/mL, about 70 mg/mL to about 230 mg/mL, about 75 mg/mL to about 230 mg/mL, about 80 mg/mL to about 230 mg/mL, about 85 mg/mL to about 230 mg/mL, about 90 mg/mL to about 230 mg/mL, about 95 mg/mL to about 230 mg/mL, about 100 mg/mL to about 230 mg/mL, about 105 mg/mL to about 230 mg/mL, about 110 mg/mL to about 230 mg/mL, about 115 mg/mL to about 230 mg/mL, about 120 mg/mL to about 230 mg/mL, about 125 mg/mL to about 230 mg/mL, about 130 mg/mL to about 230 mg/mL, about 135 mg/mL to about 230 mg/mL, about 140 mg/mL to about 230 mg/mL, about 145 mg/mL to about 230 mg/mL, about 150 mg/mL to about 230 mg/mL, about 155 mg/mL to about 230 mg/mL, about 160 mg/mL to about 230 mg/mL, about 165 mg/mL to about 230 mg/mL, about 170 mg/mL to about 230 mg/mL, about 175 mg/mL to about 230 mg/mL, about 180 mg/mL to about 230 mg/mL, about 185 mg/mL to about 230 mg/mL, about 190 mg/mL to about 230 mg/mL, about 195 mg/mL to about 230 mg/mL, about 200 mg/mL to about 230 mg/mL, about 205 mg/mL to about 230 mg/mL, about 210 mg/mL to about 230 mg/mL, about 215 mg/mL to about 230 mg/mL, about 220 mg/mL to about 230 mg/mL, about 225 mg/mL to about 230 mg/mL, about 50 mg/mL to about 220 mg/mL, about 55 mg/mL to about 220 mg/mL, about 60 mg/mL to about 220 mg/mL, about 65 mg/mL to about 220 mg/mL, about 70 mg/mL to about 220 mg/mL, about 75 mg/mL to about 220 mg/mL, about 80 mg/mL to about 220 mg/mL, about 85 mg/mL to about 220 mg/mL, about 90 mg/mL to about 220 mg/mL, about 95 mg/mL to about 220 mg/mL, about 100 mg/mL to about 220 mg/mL, about 105 mg/mL to about 220 mg/mL, about 110 mg/mL to about 220 mg/mL, about 115 mg/mL to about 220 mg/mL, about 120 mg/mL to about 220 mg/mL, about 125 mg/mL to about 220 mg/mL, about 130 mg/mL to about 220 mg/mL, about 135 mg/mL to about 220 mg/mL, about 140 mg/mL to about 220 mg/mL, about 145 mg/mL to about 220 mg/mL, about 150 mg/mL to about 220 mg/mL, about 155 mg/mL to about 220 mg/mL, about 160 mg/mL to about 220 mg/mL, about 165 mg/mL to about 220 mg/mL, about 170 mg/mL to about 220 mg/mL, about 175 mg/mL to about 220 mg/mL, about 180 mg/mL to about 220 mg/mL, about 185 mg/mL to about 220 mg/mL, about 190 mg/mL to about 220 mg/mL, about 195 mg/mL to about 220 mg/mL, about 200 mg/mL to about 220 mg/mL, about 205 mg/mL to about 220 mg/mL, about 210 mg/mL to about 220 mg/mL, about 215 mg/mL to about 220 mg/mL, about 50 mg/mL to about 210 mg/mL, about 55 mg/mL to about 210 mg/mL, about 60 mg/mL to about 210 mg/mL, about 65 mg/mL to about 210 mg/mL, about 70 mg/mL to about 210 mg/mL, about 75 mg/mL to about 210 mg/mL, about 80 mg/mL to about 210 mg/mL, about 85 mg/mL to about 210 mg/mL, about 90 mg/mL to about 210 mg/mL, about 95 mg/mL to about 210 mg/mL, about 100 mg/mL to about 210 mg/mL, about 105 mg/mL to about 210 mg/mL, about 110 mg/mL to about 210 mg/mL, about 115 mg/mL to about 210 mg/mL, about 120 mg/mL to about 210 mg/mL, about 125 mg/mL to about 210 mg/mL, about 130 mg/mL to about 210 mg/mL, about 135 mg/mL to about 210 mg/mL, about 140 mg/mL to about 210 mg/mL, about 145 mg/mL to about 210 mg/mL, about 150 mg/mL to about 210 mg/mL, about 155 mg/mL to about 210 mg/mL, about 160 mg/mL to about 210 mg/mL, about 165 mg/mL to about 210 mg/mL, about 170 mg/mL to about 210 mg/mL, about 175 mg/mL to about 210 mg/mL, about 180 mg/mL to about 210 mg/mL, about 185 mg/mL to about 210 mg/mL, about 190 mg/mL to about 210 mg/mL, about 195 mg/mL to about 210 mg/mL, about 200 mg/mL to about 210 mg/mL, about 205 mg/mL to about 210 mg/mL, about 50 mg/mL to about 200 mg/mL, about 55 mg/mL to about 200 mg/mL, about 60 mg/mL to about 200 mg/mL, about 65 mg/mL to about 200 mg/mL, about 70 mg/mL to about 200 mg/mL, about 75 mg/mL to about 200 mg/mL, about 80 mg/mL to about 200 mg/mL, about 85 mg/mL to about 200 mg/mL, about 90 mg/mL to about 200 mg/mL, about 95 mg/mL to about 200 mg/mL, about 100 mg/mL to about 200 mg/mL, about 105 mg/mL to about 200 mg/mL, about 110 mg/mL to about 200 mg/mL, about 115 mg/mL to about 200 mg/mL, about 120 mg/mL to about 200 mg/mL, about 125 mg/mL to about 200 mg/mL, about 130 mg/mL to about 200 mg/mL, about 135 mg/mL to about 200 mg/mL, about 140 mg/mL to about 200 mg/mL, about 145 mg/mL to about 200 mg/mL, about 150 mg/mL to about 200 mg/mL, about 155 mg/mL to about 200 mg/mL, about 160 mg/mL to about 200 mg/mL, about 165 mg/mL to about 200 mg/mL, about 170 mg/mL to about 200 mg/mL, about 175 mg/mL to about 200 mg/mL, about 180 mg/mL to about 200 mg/mL, about 185 mg/mL to about 200 mg/mL, about 190 mg/mL to about 200 mg/mL, about 195 mg/mL to about 200 mg/mL, about 50 mg/mL to about 190 mg/mL, about 55 mg/mL to about 190 mg/mL, about 60 mg/mL to about 190 mg/mL, about 65 mg/mL to about 190 mg/mL, about 70 mg/mL to about 190 mg/mL, about 75 mg/mL to about 190 mg/mL, about 80 mg/mL to about 190 mg/mL, about 85 mg/mL to about 190 mg/mL, about 90 mg/mL to about 190 mg/mL, about 95 mg/mL to about 190 mg/mL, about 100 mg/mL to about 190 mg/mL, about 105 mg/mL to about 190 mg/mL, about 110 mg/mL to about 190 mg/mL, about 115 mg/mL to about 190 mg/mL, about 120 mg/mL to about 190 mg/mL, about 125 mg/mL to about 190 mg/mL, about 130 mg/mL to about 190 mg/mL, about 135 mg/mL to about 190 mg/mL, about 140 mg/mL to about 190 mg/mL, about 145 mg/mL to about 190 mg/mL, about 150 mg/mL to about 190 mg/mL, about 155 mg/mL to about 190 mg/mL, about 160 mg/mL to about 190 mg/mL, about 165 mg/mL to about 190 mg/mL, about 170 mg/mL to about 190 mg/mL, about 175 mg/mL to about 190 mg/mL, about 180 mg/mL to about 190 mg/mL, about 185 mg/mL to about 190 mg/mL, about 50 mg/mL to about 180 mg/mL, about 55 mg/mL to about 180 mg/mL, about 60 mg/mL to about 180 mg/mL, about 65 mg/mL to about 180 mg/mL, about 70 mg/mL to about 180 mg/mL, about 75 mg/mL to about 180 mg/mL, about 80 mg/mL to about 180 mg/mL, about 85 mg/mL to about 180 mg/mL, about 90 mg/mL to about 180 mg/mL, about 95 mg/mL to about 180 mg/mL, about 100 mg/mL to about 180 mg/mL, about 105 mg/mL to about 180 mg/mL, about 110 mg/mL to about 180 mg/mL, about 115 mg/mL to about 180 mg/mL, about 120 mg/mL to about 180 mg/mL, about 125 mg/mL to about 180 mg/mL, about 130 mg/mL to about 180 mg/mL, about 135 mg/mL to about 180 mg/mL, about 140 mg/mL to about 180 mg/mL, about 145 mg/mL to about 180 mg/mL, about 150 mg/mL to about 180 mg/mL, about 155 mg/mL to about 180 mg/mL, about 160 mg/mL to about 180 mg/mL, about 165 mg/mL to about 180 mg/mL, about 170 mg/mL to about 180 mg/mL, about 175 mg/mL to about 180 mg/mL, about 50 mg/mL to about 170 mg/mL, about 55 mg/mL to about 170 mg/mL, about 60 mg/mL to about 170 mg/mL, about 65 mg/mL to about 170 mg/mL, about 70 mg/mL to about 170 mg/mL, about 75 mg/mL to about 170 mg/mL, about 80 mg/mL to about 170 mg/mL, about 85 mg/mL to about 170 mg/mL, about 90 mg/mL to about 170 mg/mL, about 95 mg/mL to about 170 mg/mL, about 100 mg/mL to about 170 mg/mL, about 105 mg/mL to about 170 mg/mL, about 110 mg/mL to about 170 mg/mL, about 115 mg/mL to about 170 mg/mL, about 120 mg/mL to about 170 mg/mL, about 125 mg/mL to about 170 mg/mL, about 130 mg/mL to about 170 mg/mL, about 135 mg/mL to about 170 mg/mL, about 140 mg/mL to about 170 mg/mL, about 145 mg/mL to about 170 mg/mL, about 150 mg/mL to about 170 mg/mL, about 155 mg/mL to about 170 mg/mL, about 160 mg/mL to about 170 mg/mL, about 165 mg/mL to about 170 mg/mL, about 50 mg/mL to about 160 mg/mL, about 55 mg/mL to about 160 mg/mL, about 60 mg/mL to about 160 mg/mL, about 65 mg/mL to about 160 mg/mL, about 70 mg/mL to about 160 mg/mL, about 75 mg/mL to about 160 mg/mL, about 80 mg/mL to about 160 mg/mL, about 85 mg/mL to about 160 mg/mL, about 90 mg/mL to about 160 mg/mL, about 95 mg/mL to about 160 mg/mL, about 100 mg/mL to about 160 mg/mL, about 105 mg/mL to about 160 mg/mL, about 110 mg/mL to about 160 mg/mL, about 115 mg/mL to about 160 mg/mL, about 120 mg/mL to about 160 mg/mL, about 125 mg/mL to about 160 mg/mL, about 130 mg/mL to about 160 mg/mL, about 135 mg/mL to about 160 mg/mL, about 140 mg/mL to about 160 mg/mL, about 145 mg/mL to about 160 mg/mL, about 150 mg/mL to about 160 mg/mL, about 155 mg/mL to about 160 mg/mL, about 50 mg/mL to about 150 mg/mL, about 55 mg/mL to about 150 mg/mL, about 60 mg/mL to about 150 mg/mL, about 65 mg/mL to about 150 mg/mL, about 70 mg/mL to about 150 mg/mL, about 75 mg/mL to about 150 mg/mL, about 80 mg/mL to about 150 mg/mL, about 85 mg/mL to about 150 mg/mL, about 90 mg/mL to about 150 mg/mL, about 95 mg/mL to about 150 mg/mL, about 100 mg/mL to about 150 mg/mL, about 105 mg/mL to about 150 mg/mL, about 110 mg/mL to about 150 mg/mL, about 115 mg/mL to about 150 mg/mL, about 120 mg/mL to about 150 mg/mL, about 125 mg/mL to about 150 mg/mL, about 130 mg/mL to about 150 mg/mL, about 135 mg/mL to about 150 mg/mL, about 140 mg/mL to about 150 mg/mL, or about 145 mg/mL to about 150 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 140 mg/mL, about 55 mg/mL to about 140 mg/mL, about 60 mg/mL to about 140 mg/mL, about 65 mg/mL to about 140 mg/mL, about 70 mg/mL to about 140 mg/mL, about 75 mg/mL to about 140 mg/mL, about 80 mg/mL to about 140 mg/mL, about 85 mg/mL to about 140 mg/mL, about 90 mg/mL to about 140 mg/mL, about 95 mg/mL to about 140 mg/mL, about 100 mg/mL to about 140 mg/mL, about 105 mg/mL to about 140 mg/mL, about 110 mg/mL to about 140 mg/mL, about 115 mg/mL to about 140 mg/mL, about 120 mg/mL to about 140 mg/mL, about 125 mg/mL to about 140 mg/mL, about 130 mg/mL to about 140 mg/mL, or about 135 mg/mL to about 140 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 130 mg/mL, about 55 mg/mL to about 130 mg/mL, about 60 mg/mL to about 130 mg/mL, about 65 mg/mL to about 130 mg/mL, about 70 mg/mL to about 130 mg/mL, about 75 mg/mL to about 130 mg/mL, about 80 mg/mL to about 130 mg/mL, about 85 mg/mL to about 130 mg/mL, about 90 mg/mL to about 130 mg/mL, about 95 mg/mL to about 130 mg/mL, about 100 mg/mL to about 130 mg/mL, about 105 mg/mL to about 130 mg/mL, about 110 mg/mL to about 130 mg/mL, about 115 mg/mL to about 130 mg/mL, about 120 mg/mL to about 130 mg/mL, or about 125 mg/mL to about 130 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 120 mg/mL, about 55 mg/mL to about 120 mg/mL, about 60 mg/mL to about 120 mg/mL, about 65 mg/mL to about 120 mg/mL, about 70 mg/mL to about 120 mg/mL, about 75 mg/mL to about 120 mg/mL, about 80 mg/mL to about 120 mg/mL, about 85 mg/mL to about 120 mg/mL, about 90 mg/mL to about 120 mg/mL, about 95 mg/mL to about 120 mg/mL, about 100 mg/mL to about 120 mg/mL, about 105 mg/mL to about 120 mg/mL, about 110 mg/mL to about 120 mg/mL, or about 115 mg/mL to about 120 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 110 mg/mL, about 55 mg/mL to about 110 mg/mL, about 60 mg/mL to about 110 mg/mL, about 65 mg/mL to about 110 mg/mL, about 70 mg/mL to about 110 mg/mL, about 75 mg/mL to about 110 mg/mL, about 80 mg/mL to about 110 mg/mL, about 85 mg/mL to about 110 mg/mL, about 90 mg/mL to about 110 mg/mL, about 95 mg/mL to about 110 mg/mL, about 100 mg/mL to about 110 mg/mL, or about 105 mg/mL to about 110 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 100 mg/mL, about 55 mg/mL to about 100 mg/mL, about 60 mg/mL to about 100 mg/mL, about 65 mg/mL to about 100 mg/mL, about 70 mg/mL to about 100 mg/mL, about 75 mg/mL to about 100 mg/mL, about 80 mg/mL to about 100 mg/mL, about 85 mg/mL to about 100 mg/mL, about 90 mg/mL to about 100 mg/mL, about 95 mg/mL to about 100 mg/mL, about 100 mg/mL to about 100 mg/mL, or about 105 mg/mL to about 100 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 90 mg/mL, about 55 mg/mL to about 90 mg/mL, about 60 mg/mL to about 90 mg/mL, about 65 mg/mL to about 90 mg/mL, about 70 mg/mL to about 90 mg/mL, about 75 mg/mL to about 90 mg/mL, about 80 mg/mL to about 90 mg/mL, or about 85 mg/mL to about 90 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 80 mg/mL, about 55 mg/mL to about 80 mg/mL, about 60 mg/mL to about 80 mg/mL, about 65 mg/mL to about 80 mg/mL, about 70 mg/mL to about 80 mg/mL, or about 75 mg/mL to about 80 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 70 mg/mL, about 55 mg/mL to about 70 mg/mL, about 60 mg/mL to about 70 mg/mL, or about 65 mg/mL to about 70 mg/mL. In some embodiments, the composition comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) at a concentration of about 50 mg/mL to about 55 mg/mL, about 50 mg/mL to about 60 mg/mL, or about 55 mg/mL to about 60 mg/mL.

The composition provided herein may have a viscosity of less than or about 20 centipoise (cP). The composition may have a viscosity of less than or about 15 centipoise (cP). The composition may have a viscosity of less than or about 10 centipoise (cP). For instance, the composition has a viscosity of less than or about 20, 19, 18, 17, 16, 15, 14, 13, 12, 11, 10, 9, 8, 7, 6, 5, 4, 3, or 2 cP. The composition may have a viscosity of at least about 1, 2 or 3 cP. Further example viscosities include about 1 cP to about 2 cP, about 1 cP to about 3 cP, about 1 cP to about 4 cP, about 1 cP to about 5 cP, about 1 cP to about 6 cP, about 1 cP to about 7 cP, about 1 cP to about 8 cP, about 1 cP to about 9 cP, about 1 cP to about 10 cP, about 1 cP to about 11 cP, about 1 cP to about 12 cP, about 1 cP to about 13 cP, about 1 cP to about 14 cP, about 1 cP to about 15 cP, about 1 cP to about 16 cP, about 1 cP to about 17 cP, about 1 cP to about 18 cP, about 1 cP to about 19 cP, about 1 cP to about 20 cP, about 2 cP to about 5 cP, about 2 cP to about 6 cP, about 2 cP to about 7 cP, about 2 cP to about 8 cP, about 2 cP to about 9 cP, about 2 cP to about 10 cP, about 2 cP to about 11 cP, about 2 cP to about 12 cP, about 2 cP to about 13 cP, about 2 cP to about 14 cP, about 2 cP to about 15 cP, about 2 cP to about 16 cP, about 2 cP to about 17 cP, about 2 cP to about 18 cP, about 2 cP to about 19 cP, about 2 cP to about 20 cP, about 3 cP to about 5 cP, about 3 cP to about 6 cP, about 3 cP to about 7 cP, about 3 cP to about 8 cP, about 3 cP to about 9 cP, about 3 cP to about 10 cP, about 3 cP to about 11 cP, about 3 cP to about 12 cP, about 3 cP to about 13 cP, about 3 cP to about 14 cP, about 3 cP to about 15 cP, about 3 cP to about 16 cP, about 3 cP to about 17 cP, about 3 cP to about 18 cP, about 3 cP to about 19 cP, about cP to about 20 cP, about 4 cP to about 5 cP, about 4 cP to about 6 cP, about 4 cP to about 7 cP, about 4 cP to about 8 cP, about 4 cP to about 9 cP, or about 4 cP to about 10 cP. about 4 cP to about 11 cP, about 4 cP to about 12 cP, about 4 cP to about 13 cP, about 4 cP to about 14 cP, about 4 cP to about 15 cP, about 4 cP to about 16 cP, about 4 cP to about 17 cP, about 4 cP to about 18 cP, about 4 cP to about 19 cP, about 4 cP to about 20 cP, about 5 cP to about 10 cP, about 5 cP to about 11 cP, about 5 cP to about 12 cP, about 5 cP to about 13 cP, about 5 cP to about 14 cP, about 5 cP to about 15 cP, about 5 cP to about 16 cP, about 5 cP to about 17 cP, about 5 cP to about 18 cP, about 5 cP to about 19 cP, about 5 cP to about 20 cP, about 6 cP to about 10 cP, about 6 cP to about 11 cP, about 6 cP to about 12 cP, about 6 cP to about 13 cP, about 6 cP to about 14 cP, about 6 cP to about 15 cP, about 6 cP to about 16 cP, about 6 cP to about 17 cP, about 6 cP to about 18 cP, about 6 cP to about 19 cP, about 6 cP to about 20 cP, about 7 cP to about 10 cP, about 7 cP to about 11 cP, about 7 cP to about 12 cP, about 7 cP to about 13 cP, about 7 cP to about 14 cP, about 7 cP to about 15 cP, about 7 cP to about 16 cP, about 7 cP to about 17 cP, about 7 cP to about 18 cP, about 7 cP to about 19 cP, about 7 cP to about 20 cP, about 8 cP to about 10 cP, about 8 cP to about 11 cP, about 8 cP to about 12 cP, about 8 cP to about 13 cP, about 8 cP to about 14 cP, about 8 cP to about 15 cP, about 8 cP to about 16 cP, about 8 cP to about 17 cP, about 8 cP to about 18 cP, about 8 cP to about 19 cP, or about 8 cP to about 20 cP. In some embodiments, a centipoise as used herein is a millipascal-second (mPa·s).

In certain embodiments, provided herein is a pharmaceutical composition comprising a therapeutically effective dose of a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) having a total volume of less than or equal to about 2.5 mL. In some embodiments, the pharmaceutical composition comprises a therapeutically effective dose of a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) having a total volume of less than or equal to about 2 mL. The total volume may be less than or equal to about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, or 0.8 mL. The total volume may be at least about 0.5 mL. The total volume may be about 0.5 mL to about 3 mL, about 0.5 mL to about 2.9 mL, about 0.5 mL to about 2.8 mL, about 0.5 mL to about 2.7 mL, about 0.5 mL to about 2.6 mL, about 0.5 mL to about 2.5 mL, about 0.5 mL to about 2.4 mL, about 0.5 mL to about 2.3 mL, about 0.5 mL to about 2.2 mL, about 0.5 mL to about 2.1 mL, about 0.5 mL to about 2.0 mL, about 0.5 mL to about 1.9 mL, about 0.5 mL to about 1.8 mL, about 0.5 mL to about 1.7 mL, about 0.5 mL to about 1.6 mL, about 0.5 mL to about 1.5 mL, about 0.5 mL to about 1.4 mL, about 0.5 mL to about 1.3 mL, about 0.5 mL to about 1.2 mL, about 0.5 mL to about 1.1 mL, about 0.5 mL to about 1.0 mL, about 0.5 mL to about 0.9 mL, about 0.5 mL to about 0.8 mL, about 0.6 mL to about 3 mL, about 0.6 mL to about 2.9 mL, about 0.6 mL to about 2.8 mL, about 0.6 mL to about 2.7 mL, about 0.6 mL to about 2.6 mL, about 0.6 mL to about 2.5 mL, about 0.6 mL to about 2.4 mL, about 0.6 mL to about 2.3 mL, about 0.6 mL to about 2.2 mL, about 0.6 mL to about 2.1 mL, about 0.6 mL to about 2.0 mL, about 0.6 mL to about 1.9 mL, about 0.6 mL to about 1.8 mL, about 0.6 mL to about 1.7 mL, about 0.6 mL to about 1.6 mL, 0.6 mL to about 1.5 mL, about 0.6 mL to about 1.4 mL, about 0.6 mL to about 1.3 mL, about 0.6 mL to about 1.2 mL, about 0.6 mL to about 1.1 mL, about 0.6 mL to about 1.0 mL, about 0.6 mL to about 0.9 mL, about 0.6 mL to about 0.8 mL, about 0.7 mL to about 3 mL, about 0.7 mL to about 2.9 mL, about 0.7 mL to about 2.8 mL, about 0.7 mL to about 2.7 mL, about 0.7 mL to about 2.6 mL, about 0.7 mL to about 2.5 mL, about 0.7 mL to about 2.4 mL, about 0.7 mL to about 2.3 mL, about 0.7 mL to about 2.2 mL, about 0.7 mL to about 2.1 mL, about 0.7 mL to about 2.0 mL, about 0.7 mL to about 1.9 mL, about 0.7 mL to about 1.8 mL, about 0.7 mL to about 1.7 mL, about 0.7 mL to about 1.6 mL, about 0.7 mL to about 1.5 mL, about 0.7 mL to about 1.4 mL, about 0.7 mL to about 1.3 mL, about 0.7 mL to about 1.2 mL, about 0.7 mL to about 1.1 mL, about 0.7 mL to about 1.0 mL, about 0.7 mL to about 0.9 mL, about 0.7 mL to about 0.8 mL, about 3 mL to about 10 mL, about 3 mL to about 9.5 mL, about 3 mL to about 9.0 mL, about 3 mL to about 8.5 mL, about 3 mL to about 8.0 mL, about 3 mL to about 7.5 mL, about 3 mL to about 7.0 mL, about 3 mL to about 6.5 mL, about 3 mL to about 6 mL, about 3 mL to about 5.5 mL, about 3 mL to about 5.0 mL, about 3 mL to about 4.5 mL, about 3 mL to about 4 mL, about 3 mL to about 3.5 mL, about 3.5 mL to about 10 mL, about 3.5 mL to about 9.5 mL, about 3.5 mL to about 9.0 mL, about 3.5 mL to about 8.5 mL, about 3.5 mL to about 8.0 mL, about 3.5 mL to about 7.5 mL, about 3.5 mL to about 7.0 mL, about 3.5 mL to about 6.5 mL, about 3.5 mL to about 6 mL, about 3.5 mL to about 5.5 mL, about 3.5 mL to about 5.0 mL, about 3.5 mL to about 4.5 mL, about 3.5 mL to about 4 mL, about 4.0 mL to about 10 mL, about 4.0 mL to about 9.5 mL, about 4.0 mL to about 9.0 mL, about 4.0 mL to about 8.5 mL, about 4.0 mL to about 8.0 mL, about 4.0 mL to about 7.5 mL, about 4.0 mL to about 7.0 mL, about 4.0 mL to about 6.5 mL, about 4.0 mL to about 6 mL, about 4.0 mL to about 5.5 mL, about 4.0 mL to about 5.0 mL, about 4.0 mL to about 4.5 mL, about 4.5 mL to about 10 mL, about 4.5 mL to about 9.5 mL, about 4.5 mL to about 9.0 mL, about 4.5 mL to about 8.5 mL, about 4.5 mL to about 8.0 mL, about 4.5 mL to about 7.5 mL, about 4.5 mL to about 7.0 mL, about 4.5 mL to about 6.5 mL, about 4.5 mL to about 6 mL, about 4.5 mL to about 5.5 mL, about 4.5 mL to about 5.0 mL, about 5 mL to about 10 mL, about 5 mL to about 9.5 mL, about 5 mL to about 9.0 mL, about 5 mL to about 8.5 mL, about 5 mL to about 8.0 mL, about 5 mL to about 7.5 mL, about 5 mL to about 7.0 mL, about 5 mL to about 6.5 mL, about 5 mL to about 6 mL, about 5 mL to about 5.5 mL, about 5.5 mL to about 10 mL, about 5.5 mL to about 9.5 mL, about 5.5 mL to about 9.0 mL, about 5.5 mL to about 8.5 mL, about 5.5 mL to about 8.0 mL, about 5.5 mL to about 7.5 mL, about 5.5 mL to about 7.0 mL, about 5.5 mL to about 6.5 mL, about 5.5 mL to about 6 mL, about 6.0 mL to about 10 mL, about 6.0 mL to about 9.5 mL, about 6.0 mL to about 9.0 mL, about 6.0 mL to about 8.5 mL, about 6.0 mL to about 8.0 mL, about 6.0 mL to about 7.5 mL, about 6.0 mL to about 7.0 mL, about 6.0 mL to about 6.5 mL, about 6.5 mL to about 10 mL, about 6.5 mL to about 9.5 mL, about 6.5 mL to about 9.0 mL, about 6.5 mL to about 8.5 mL, about 6.5 mL to about 8.0 mL, about 6.5 mL to about 7.5 mL, about 6.5 mL to about 7.0 mL, about 7.0 mL to about 10 mL, about 7.0 mL to about 9.5 mL, about 7.0 mL to about 9.0 mL, about 7.0 mL to about 8.5 mL, about 7.0 mL to about 8.0 mL, about 7.0 mL to about 7.5 mL, about 7.5 mL to about 10 mL, about 7.5 mL to about 9.5 mL, about 7.5 mL to about 9.0 mL, about 7.5 mL to about 8.5 mL, about 7.5 mL to about 8.0 mL, about 8.0 mL to about 10 mL, about 8.0 mL to about 9.5 mL, about 8.0 mL to about 9.0 mL, about 8.0 mL to about 8.5 mL, about 8.5 mL to about 10 mL, about 8.5 mL to about 9.5 mL, about 8.5 mL to about 9.0 mL, about 9 mL to about 10 mL, about 9 mL to about 9.5 mL, or about 9.5 mL to about 10 mL. The composition may have a viscosity of less than or about 10 centipoise (cP). For instance, the composition has a viscosity of less than or about 20, 19, 18, 17, 16, 15, 14, 13, 12, 11, 10, 9, 8, 7, 6, 5, 4, 3, or 2 cP. The composition may have a viscosity of at least about 1, 2 or 3 cP. Further example viscosities include about 1 cP to about 2 cP, about 1 cP to about 3 cP, about 1 cP to about 4 cP, about 1 cP to about 5 cP, about 1 cP to about 6 cP, about 1 cP to about 7 cP, about 1 cP to about 8 cP, about 1 cP to about 9 cP, about 1 cP to about 10 cP, about 2 cP to about 5 cP, about 2 cP to about 6 cP, about 2 cP to about 7 cP, about 2 cP to about 8 cP, about 2 cP to about 9 cP, about 2 cP to about 10 cP, about 3 cP to about 5 cP, about 3 cP to about 6 cP, about 3 cP to about 7 cP, about 3 cP to about 8 cP, about 3 cP to about 9 cP, about 3 cP to about 10 cP, about 4 cP to about 5 cP, about 4 cP to about 6 cP, about 4 cP to about 7 cP, about 4 cP to about 8 cP, about 4 cP to about 9 cP, or about 4 cP to about 10 cP, In some embodiments, the therapeutically effective dose is at least about 150 mg TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2). In some cases, the therapeutically effective dose is about or at least about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of anti-TL1A or anti-IL23. In some cases, the therapeutically effective dose is about 150 mg to about 2000 mg, about 150 mg to about 1750 mg, about 150 mg to about 1500 mg, about 150 mg to about 1250 mg, about 150 mg to about 1000 mg, about 150 mg to about 750 mg, about 150 mg to about 500 mg, about 150 mg to about 450 mg, about 150 mg to about 400 mg, about 150 mg to about 350 mg, about 150 mg to about 300 mg, about 150 mg to about 250 mg, or about 150 mg to about 200 mg anti-TL1A or anti-IL23. In some embodiments, the pharmaceutical composition comprises about 50 mg/mL to about 250 mg/mL, about 55 mg/mL to about 250 mg/mL, about 60 mg/mL to about 250 mg/mL, about 65 mg/mL to about 250 mg/mL, about 70 mg/mL to about 250 mg/mL, about 75 mg/mL to about 250 mg/mL, about 80 mg/mL to about 250 mg/mL, about 85 mg/mL to about 250 mg/mL, about 90 mg/mL to about 250 mg/mL, about 95 mg/mL to about 250 mg/mL, about 100 mg/mL to about 250 mg/mL, about 105 mg/mL to about 250 mg/mL, about 110 mg/mL to about 250 mg/mL, about 115 mg/mL to about 250 mg/mL, about 120 mg/mL to about 250 mg/mL, about 125 mg/mL to about 250 mg/mL, about 130 mg/mL to about 250 mg/mL, about 135 mg/mL to about 250 mg/mL, about 140 mg/mL to about 250 mg/mL, about 145 mg/mL to about 250 mg/mL, about 150 mg/mL to about 250 mg/mL, about 155 mg/mL to about 250 mg/mL, about 160 mg/mL to about 250 mg/mL, about 165 mg/mL to about 250 mg/mL, about 170 mg/mL to about 250 mg/mL, about 175 mg/mL to about 250 mg/mL, about 180 mg/mL to about 250 mg/mL, about 185 mg/mL to about 250 mg/mL, about 190 mg/mL to about 250 mg/mL, about 195 mg/mL to about 250 mg/mL, about 200 mg/mL to about 250 mg/mL, about 205 mg/mL to about 250 mg/mL, about 210 mg/mL to about 250 mg/mL, about 215 mg/mL to about 250 mg/mL, about 220 mg/mL to about 250 mg/mL, about 225 mg/mL to about 250 mg/mL, about 230 mg/mL to about 250 mg/mL, about 235 mg/mL to about 250 mg/mL, about 240 mg/mL to about 250 mg/mL, about 245 mg/mL to about 250 mg/mL, about 50 mg/mL to about 240 mg/mL, about 55 mg/mL to about 240 mg/mL, about 60 mg/mL to about 240 mg/mL, about 65 mg/mL to about 240 mg/mL, about 70 mg/mL to about 240 mg/mL, about 75 mg/mL to about 240 mg/mL, about 80 mg/mL to about 240 mg/mL, about 85 mg/mL to about 240 mg/mL, about 90 mg/mL to about 240 mg/mL, about 95 mg/mL to about 240 mg/mL, about 100 mg/mL to about 240 mg/mL, about 105 mg/mL to about 240 mg/mL, about 110 mg/mL to about 240 mg/mL, about 115 mg/mL to about 240 mg/mL, about 120 mg/mL to about 240 mg/mL, about 125 mg/mL to about 240 mg/mL, about 130 mg/mL to about 240 mg/mL, about 135 mg/mL to about 240 mg/mL, about 140 mg/mL to about 240 mg/mL, about 145 mg/mL to about 240 mg/mL, about 150 mg/mL to about 240 mg/mL, about 155 mg/mL to about 240 mg/mL, about 160 mg/mL to about 240 mg/mL, about 165 mg/mL to about 240 mg/mL, about 170 mg/mL to about 240 mg/mL, about 175 mg/mL to about 240 mg/mL, about 180 mg/mL to about 240 mg/mL, about 185 mg/mL to about 240 mg/mL, about 190 mg/mL to about 240 mg/mL, about 195 mg/mL to about 240 mg/mL, about 200 mg/mL to about 240 mg/mL, about 205 mg/mL to about 240 mg/mL, about 210 mg/mL to about 240 mg/mL, about 215 mg/mL to about 240 mg/mL, about 220 mg/mL to about 240 mg/mL, about 225 mg/mL to about 240 mg/mL, about 230 mg/mL to about 240 mg/mL, about 235 mg/mL to about 240 mg/mL, about 50 mg/mL to about 230 mg/mL, about 55 mg/mL to about 230 mg/mL, about 60 mg/mL to about 230 mg/mL, about 65 mg/mL to about 230 mg/mL, about 70 mg/mL to about 230 mg/mL, about 75 mg/mL to about 230 mg/mL, about 80 mg/mL to about 230 mg/mL, about 85 mg/mL to about 230 mg/mL, about 90 mg/mL to about 230 mg/mL, about 95 mg/mL to about 230 mg/mL, about 100 mg/mL to about 230 mg/mL, about 105 mg/mL to about 230 mg/mL, about 110 mg/mL to about 230 mg/mL, about 115 mg/mL to about 230 mg/mL, about 120 mg/mL to about 230 mg/mL, about 125 mg/mL to about 230 mg/mL, about 130 mg/mL to about 230 mg/mL, about 135 mg/mL to about 230 mg/mL, about 140 mg/mL to about 230 mg/mL, about 145 mg/mL to about 230 mg/mL, about 150 mg/mL to about 230 mg/mL, about 155 mg/mL to about 230 mg/mL, about 160 mg/mL to about 230 mg/mL, about 165 mg/mL to about 230 mg/mL, about 170 mg/mL to about 230 mg/mL, about 175 mg/mL to about 230 mg/mL, about 180 mg/mL to about 230 mg/mL, about 185 mg/mL to about 230 mg/mL, about 190 mg/mL to about 230 mg/mL, about 195 mg/mL to about 230 mg/mL, about 200 mg/mL to about 230 mg/mL, about 205 mg/mL to about 230 mg/mL, about 210 mg/mL to about 230 mg/mL, about 215 mg/mL to about 230 mg/mL, about 220 mg/mL to about 230 mg/mL, about 225 mg/mL to about 230 mg/mL, about 50 mg/mL to about 220 mg/mL, about 55 mg/mL to about 220 mg/mL, about 60 mg/mL to about 220 mg/mL, about 65 mg/mL to about 220 mg/mL, about 70 mg/mL to about 220 mg/mL, about 75 mg/mL to about 220 mg/mL, about 80 mg/mL to about 220 mg/mL, about 85 mg/mL to about 220 mg/mL, about 90 mg/mL to about 220 mg/mL, about 95 mg/mL to about 220 mg/mL, about 100 mg/mL to about 220 mg/mL, about 105 mg/mL to about 220 mg/mL, about 110 mg/mL to about 220 mg/mL, about 115 mg/mL to about 220 mg/mL, about 120 mg/mL to about 220 mg/mL, about 125 mg/mL to about 220 mg/mL, about 130 mg/mL to about 220 mg/mL, about 135 mg/mL to about 220 mg/mL, about 140 mg/mL to about 220 mg/mL, about 145 mg/mL to about 220 mg/mL, about 150 mg/mL to about 220 mg/mL, about 155 mg/mL to about 220 mg/mL, about 160 mg/mL to about 220 mg/mL, about 165 mg/mL to about 220 mg/mL, about 170 mg/mL to about 220 mg/mL, about 175 mg/mL to about 220 mg/mL, about 180 mg/mL to about 220 mg/mL, about 185 mg/mL to about 220 mg/mL, about 190 mg/mL to about 220 mg/mL, about 195 mg/mL to about 220 mg/mL, about 200 mg/mL to about 220 mg/mL, about 205 mg/mL to about 220 mg/mL, about 210 mg/mL to about 220 mg/mL, about 215 mg/mL to about 220 mg/mL, about 50 mg/mL to about 210 mg/mL, about 55 mg/mL to about 210 mg/mL, about 60 mg/mL to about 210 mg/mL, about 65 mg/mL to about 210 mg/mL, about 70 mg/mL to about 210 mg/mL, about 75 mg/mL to about 210 mg/mL, about 80 mg/mL to about 210 mg/mL, about 85 mg/mL to about 210 mg/mL, about 90 mg/mL to about 210 mg/mL, about 95 mg/mL to about 210 mg/mL, about 100 mg/mL to about 210 mg/mL, about 105 mg/mL to about 210 mg/mL, about 110 mg/mL to about 210 mg/mL, about 115 mg/mL to about 210 mg/mL, about 120 mg/mL to about 210 mg/mL, about 125 mg/mL to about 210 mg/mL, about 130 mg/mL to about 210 mg/mL, about 135 mg/mL to about 210 mg/mL, about 140 mg/mL to about 210 mg/mL, about 145 mg/mL to about 210 mg/mL, about 150 mg/mL to about 210 mg/mL, about 155 mg/mL to about 210 mg/mL, about 160 mg/mL to about 210 mg/mL, about 165 mg/mL to about 210 mg/mL, about 170 mg/mL to about 210 mg/mL, about 175 mg/mL to about 210 mg/mL, about 180 mg/mL to about 210 mg/mL, about 185 mg/mL to about 210 mg/mL, about 190 mg/mL to about 210 mg/mL, about 195 mg/mL to about 210 mg/mL, about 200 mg/mL to about 210 mg/mL, about 205 mg/mL to about 210 mg/mL, about 50 mg/mL to about 200 mg/mL, about 55 mg/mL to about 200 mg/mL, about 60 mg/mL to about 200 mg/mL, about 65 mg/mL to about 200 mg/mL, about 70 mg/mL to about 200 mg/mL, about 75 mg/mL to about 200 mg/mL, about 80 mg/mL to about 200 mg/mL, about 85 mg/mL to about 200 mg/mL, about 90 mg/mL to about 200 mg/mL, about 95 mg/mL to about 200 mg/mL, about 100 mg/mL to about 200 mg/mL, about 105 mg/mL to about 200 mg/mL, about 110 mg/mL to about 200 mg/mL, about 115 mg/mL to about 200 mg/mL, about 120 mg/mL to about 200 mg/mL, about 125 mg/mL to about 200 mg/mL, about 130 mg/mL to about 200 mg/mL, about 135 mg/mL to about 200 mg/mL, about 140 mg/mL to about 200 mg/mL, about 145 mg/mL to about 200 mg/mL, about 150 mg/mL to about 200 mg/mL, about 155 mg/mL to about 200 mg/mL, about 160 mg/mL to about 200 mg/mL, about 165 mg/mL to about 200 mg/mL, about 170 mg/mL to about 200 mg/mL, about 175 mg/mL to about 200 mg/mL, about 180 mg/mL to about 200 mg/mL, about 185 mg/mL to about 200 mg/mL, about 190 mg/mL to about 200 mg/mL, about 195 mg/mL to about 200 mg/mL, about 50 mg/mL to about 190 mg/mL, about 55 mg/mL to about 190 mg/mL, about 60 mg/mL to about 190 mg/mL, about 65 mg/mL to about 190 mg/mL, about 70 mg/mL to about 190 mg/mL, about 75 mg/mL to about 190 mg/mL, about 80 mg/mL to about 190 mg/mL, about 85 mg/mL to about 190 mg/mL, about 90 mg/mL to about 190 mg/mL, about 95 mg/mL to about 190 mg/mL, about 100 mg/mL to about 190 mg/mL, about 105 mg/mL to about 190 mg/mL, about 110 mg/mL to about 190 mg/mL, about 115 mg/mL to about 190 mg/mL, about 120 mg/mL to about 190 mg/mL, about 125 mg/mL to about 190 mg/mL, about 130 mg/mL to about 190 mg/mL, about 135 mg/mL to about 190 mg/mL, about 140 mg/mL to about 190 mg/mL, about 145 mg/mL to about 190 mg/mL, about 150 mg/mL to about 190 mg/mL, about 155 mg/mL to about 190 mg/mL, about 160 mg/mL to about 190 mg/mL, about 165 mg/mL to about 190 mg/mL, about 170 mg/mL to about 190 mg/mL, about 175 mg/mL to about 190 mg/mL, about 180 mg/mL to about 190 mg/mL, about 185 mg/mL to about 190 mg/mL, about 50 mg/mL to about 180 mg/mL, about 55 mg/mL to about 180 mg/mL, about 60 mg/mL to about 180 mg/mL, about 65 mg/mL to about 180 mg/mL, about 70 mg/mL to about 180 mg/mL, about 75 mg/mL to about 180 mg/mL, about 80 mg/mL to about 180 mg/mL, about 85 mg/mL to about 180 mg/mL, about 90 mg/mL to about 180 mg/mL, about 95 mg/mL to about 180 mg/mL, about 100 mg/mL to about 180 mg/mL, about 105 mg/mL to about 180 mg/mL, about 110 mg/mL to about 180 mg/mL, about 115 mg/mL to about 180 mg/mL, about 120 mg/mL to about 180 mg/mL, about 125 mg/mL to about 180 mg/mL, about 130 mg/mL to about 180 mg/mL, about 135 mg/mL to about 180 mg/mL, about 140 mg/mL to about 180 mg/mL, about 145 mg/mL to about 180 mg/mL, about 150 mg/mL to about 180 mg/mL, about 155 mg/mL to about 180 mg/mL, about 160 mg/mL to about 180 mg/mL, about 165 mg/mL to about 180 mg/mL, about 170 mg/mL to about 180 mg/mL, about 175 mg/mL to about 180 mg/mL, about 50 mg/mL to about 170 mg/mL, about 55 mg/mL to about 170 mg/mL, about 60 mg/mL to about 170 mg/mL, about 65 mg/mL to about 170 mg/mL, about 70 mg/mL to about 170 mg/mL, about 75 mg/mL to about 170 mg/mL, about 80 mg/mL to about 170 mg/mL, about 85 mg/mL to about 170 mg/mL, about 90 mg/mL to about 170 mg/mL, about 95 mg/mL to about 170 mg/mL, about 100 mg/mL to about 170 mg/mL, about 105 mg/mL to about 170 mg/mL, about 110 mg/mL to about 170 mg/mL, about 115 mg/mL to about 170 mg/mL, about 120 mg/mL to about 170 mg/mL, about 125 mg/mL to about 170 mg/mL, about 130 mg/mL to about 170 mg/mL, about 135 mg/mL to about 170 mg/mL, about 140 mg/mL to about 170 mg/mL, about 145 mg/mL to about 170 mg/mL, about 150 mg/mL to about 170 mg/mL, about 155 mg/mL to about 170 mg/mL, about 160 mg/mL to about 170 mg/mL, about 165 mg/mL to about 170 mg/mL, about 50 mg/mL to about 160 mg/mL, about 55 mg/mL to about 160 mg/mL, about 60 mg/mL to about 160 mg/mL, about 65 mg/mL to about 160 mg/mL, about 70 mg/mL to about 160 mg/mL, about 75 mg/mL to about 160 mg/mL, about 80 mg/mL to about 160 mg/mL, about 85 mg/mL to about 160 mg/mL, about 90 mg/mL to about 160 mg/mL, about 95 mg/mL to about 160 mg/mL, about 100 mg/mL to about 160 mg/mL, about 105 mg/mL to about 160 mg/mL, about 110 mg/mL to about 160 mg/mL, about 115 mg/mL to about 160 mg/mL, about 120 mg/mL to about 160 mg/mL, about 125 mg/mL to about 160 mg/mL, about 130 mg/mL to about 160 mg/mL, about 135 mg/mL to about 160 mg/mL, about 140 mg/mL to about 160 mg/mL, about 145 mg/mL to about 160 mg/mL, about 150 mg/mL to about 160 mg/mL, about 155 mg/mL to about 160 mg/mL, about 50 mg/mL to about 150 mg/mL, about 55 mg/mL to about 150 mg/mL, about 60 mg/mL to about 150 mg/mL, about 65 mg/mL to about 150 mg/mL, about 70 mg/mL to about 150 mg/mL, about 75 mg/mL to about 150 mg/mL, about 80 mg/mL to about 150 mg/mL, about 85 mg/mL to about 150 mg/mL, about 90 mg/mL to about 150 mg/mL, about 95 mg/mL to about 150 mg/mL, about 100 mg/mL to about 150 mg/mL, about 105 mg/mL to about 150 mg/mL, about 110 mg/mL to about 150 mg/mL, about 115 mg/mL to about 150 mg/mL, about 120 mg/mL to about 150 mg/mL, about 125 mg/mL to about 150 mg/mL, about 130 mg/mL to about 150 mg/mL, about 135 mg/mL to about 150 mg/mL, about 140 mg/mL to about 150 mg/mL, about 145 mg/mL to about 150 mg/mL, about 50 mg/mL to about 140 mg/mL, about 55 mg/mL to about 140 mg/mL, about 60 mg/mL to about 140 mg/mL, about 65 mg/mL to about 140 mg/mL, about 70 mg/mL to about 140 mg/mL, about 75 mg/mL to about 140 mg/mL, about 80 mg/mL to about 140 mg/mL, about 85 mg/mL to about 140 mg/mL, about 90 mg/mL to about 140 mg/mL, about 95 mg/mL to about 140 mg/mL, about 100 mg/mL to about 140 mg/mL, about 105 mg/mL to about 140 mg/mL, about 110 mg/mL to about 140 mg/mL, about 115 mg/mL to about 140 mg/mL, about 120 mg/mL to about 140 mg/mL, about 125 mg/mL to about 140 mg/mL, about 130 mg/mL to about 140 mg/mL, about 135 mg/mL to about 140 mg/mL, about 50 mg/mL to about 130 mg/mL, about 55 mg/mL to about 130 mg/mL, about 60 mg/mL to about 130 mg/mL, about 65 mg/mL to about 130 mg/mL, about 70 mg/mL to about 130 mg/mL, about 75 mg/mL to about 130 mg/mL, about 80 mg/mL to about 130 mg/mL, about 85 mg/mL to about 130 mg/mL, about 90 mg/mL to about 130 mg/mL, about 95 mg/mL to about 130 mg/mL, about 100 mg/mL to about 130 mg/mL, about 105 mg/mL to about 130 mg/mL, about 110 mg/mL to about 130 mg/mL, about 115 mg/mL to about 130 mg/mL, about 120 mg/mL to about 130 mg/mL, or about 125 mg/mL to about 130 mg/mL, about 50 mg/mL to about 120 mg/mL, about 55 mg/mL to about 120 mg/mL, about 60 mg/mL to about 120 mg/mL, about 65 mg/mL to about 120 mg/mL, about 70 mg/mL to about 120 mg/mL, about 75 mg/mL to about 120 mg/mL, about 80 mg/mL to about 120 mg/mL, about 85 mg/mL to about 120 mg/mL, about 90 mg/mL to about 120 mg/mL, about 95 mg/mL to about 120 mg/mL, about 100 mg/mL to about 120 mg/mL, about 105 mg/mL to about 120 mg/mL, about 110 mg/mL to about 120 mg/mL, about 115 mg/mL to about 120 mg/mL, about 50 mg/mL to about 110 mg/mL, about 55 mg/mL to about 110 mg/mL, about 60 mg/mL to about 110 mg/mL, about 65 mg/mL to about 110 mg/mL, about 70 mg/mL to about 110 mg/mL, about 75 mg/mL to about 110 mg/mL, about 80 mg/mL to about 110 mg/mL, about 85 mg/mL to about 110 mg/mL, about 90 mg/mL to about 110 mg/mL, about 95 mg/mL to about 110 mg/mL, about 100 mg/mL to about 110 mg/mL, about 105 mg/mL to about 110 mg/mL, about 50 mg/mL to about 100 mg/mL, about 55 mg/mL to about 100 mg/mL, about 60 mg/mL to about 100 mg/mL, about 65 mg/mL to about 100 mg/mL, about 70 mg/mL to about 100 mg/mL, about 75 mg/mL to about 100 mg/mL, about 80 mg/mL to about 100 mg/mL, about 85 mg/mL to about 100 mg/mL, about 90 mg/mL to about 100 mg/mL, about 95 mg/mL to about 100 mg/mL, about 100 mg/mL to about 100 mg/mL, about 105 mg/mL to about 100 mg/mL, about 50 mg/mL to about 90 mg/mL, about 55 mg/mL to about 90 mg/mL, about 60 mg/mL to about 90 mg/mL, about 65 mg/mL to about 90 mg/mL, about 70 mg/mL to about 90 mg/mL, about 75 mg/mL to about 90 mg/mL, about 80 mg/mL to about 90 mg/mL, about 85 mg/mL to about 90 mg/mL, about 50 mg/mL to about 80 mg/mL, about 55 mg/mL to about 80 mg/mL, about 60 mg/mL to about 80 mg/mL, about 65 mg/mL to about 80 mg/mL, about 70 mg/mL to about 80 mg/mL, about 75 mg/mL to about 80 mg/mL, about 50 mg/mL to about 70 mg/mL, about 55 mg/mL to about 70 mg/mL, about 60 mg/mL to about 70 mg/mL, about 65 mg/mL to about 70 mg/mL, about 50 mg/mL to about 60 mg/mL, about 55 mg/mL to about 60 mg/mL, or about 50 mg/mL to about 55 mg/mL anti-TL1A or anti-IL23. In some embodiments, the concentration of anti-TL1A or anti-IL23 is about or greater than about 55, 60, 65, 70, 75, 80, 85, 90, 95, 100, 105, 110, 115, 120, 125, 130, 135, 140, 145, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg/mL.

In certain embodiments, provided herein is a pharmaceutical composition for subcutaneous administration comprising a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2), wherein at least about 150 mg of the TL1A inhibitor or the IL23 inhibitor is present in the composition. For instance, about 150 mg to about 2000 mg, about 150 mg to about 1750 mg, about 150 mg to about 1500 mg, about 150 mg to about 1250 mg, about 150 mg to about 1000 mg, about 150 mg to about 750 mg, about 150 to about 500 mg, about 150 to about 300 mg, about 150 to about 200 mg, or about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg is present in the composition. In some embodiments, up to about 2000 mg, up to about 1750 mg, up to about 1500 mg, up to about 1250 mg, up to about 1000 mg, up to about 750 mg, up to about 500 mg of anti-TL1A or IL-23 is present in the composition. The total volume of the composition may be less than or equal to about 2 mL. The total volume of the composition may be less than or equal to about 2.5 mL. The total volume may be less than about or equal to about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, or 0.8 mL. The total volume may be at least about 0.5 mL. The total volume may be about 0.5 mL to about 3 mL, about 0.5 mL to about 2.9 mL, about 0.5 mL to about 2.8 mL, about 0.5 mL to about 2.7 mL, about 0.5 mL to about 2.6 mL, about 0.5 mL to about 2.5 mL, about 0.5 mL to about 2.4 mL, about 0.5 mL to about 2.3 mL, about 0.5 mL to about 2.2 mL, about 0.5 mL to about 2.1 mL, about 0.5 mL to about 2 mL, 0.5 mL to about 1.9 mL, 0.5 mL to about 1.8 mL, 0.5 mL to about 1.7 mL, 0.5 mL to about 1.6 mL, about 0.5 mL to about 1.5 mL, about 0.5 mL to about 1.4 mL, about 0.5 mL to about 1.3 mL, about 0.5 mL to about 1.2 mL, about 0.5 mL to about 1.1 mL, about 0.5 mL to about 1.0 mL, about 0.5 mL to about 0.9 mL, about 0.5 mL to about 0.8 mL, about 0.6 mL to about 3 mL, about 0.6 mL to about 2.9 mL, about 0.6 mL to about 2.8 mL, about 0.6 mL to about 2.7 mL, about 0.6 mL to about 2.6 mL, about 0.6 mL to about 2.5 mL, about 0.6 mL to about 2.4 mL, about 0.6 mL to about 2.3 mL, about 0.6 mL to about 2.2 mL, about 0.6 mL to about 2.1 mL, about 0.6 mL to about 2.0 mL, about 0.6 mL to about 1.9 mL, about 0.6 mL to about 1.8 mL, about 0.6 mL to about 1.7 mL, about 0.6 mL to about 1.6 mL, about 0.6 mL to about 1.5 mL, about 0.6 mL to about 1.4 mL, about 0.6 mL to about 1.3 mL, about 0.6 mL to about 1.2 mL, about 0.6 mL to about 1.1 mL, about 0.6 mL to about 1.0 mL, about 0.6 mL to about 0.9 mL, about 0.6 mL to about 0.8 mL, about 0.7 mL to about 3 mL, about 0.7 mL to about 2.9 mL, about 0.7 mL to about 2.8 mL, about 0.7 mL to about 2.7 mL, about 0.7 mL to about 2.6 mL, about 0.7 mL to about 2.5 mL, about 0.7 mL to about 2.4 mL, about 0.7 mL to about 2.3 mL, about 0.7 mL to about 2.2 mL, about 0.7 mL to about 2.1 mL, about 0.7 mL to about 2.0 mL, about 0.7 mL to about 1.9 mL, about 0.7 mL to about 1.8 mL, about 0.7 mL to about 1.7 mL, about 0.7 mL to about 1.6 mL, about 0.7 mL to about 1.5 mL, about 0.7 mL to about 1.4 mL, about 0.7 mL to about 1.3 mL, about 0.7 mL to about 1.2 mL, about 0.7 mL to about 1.1 mL, about 0.7 mL to about 1.0 mL, about 0.7 mL to about 0.9 mL, about 0.7 mL to about 0.8 mL, about 3 mL to about 10 mL, about 3 mL to about 9.5 mL, about 3 mL to about 9.0 mL, about 3 mL to about 8.5 mL, about 3 mL to about 8.0 mL, about 3 mL to about 7.5 mL, about 3 mL to about 7.0 mL, about 3 mL to about 6.5 mL, about 3 mL to about 6 mL, about 3 mL to about 5.5 mL, about 3 mL to about 5.0 mL, about 3 mL to about 4.5 mL, about 3 mL to about 4 mL, about 3 mL to about 3.5 mL, about 3.5 mL to about 10 mL, about 3.5 mL to about 9.5 mL, about 3.5 mL to about 9.0 mL, about 3.5 mL to about 8.5 mL, about 3.5 mL to about 8.0 mL, about 3.5 mL to about 7.5 mL, about 3.5 mL to about 7.0 mL, about 3.5 mL to about 6.5 mL, about 3.5 mL to about 6 mL, about 3.5 mL to about 5.5 mL, about 3.5 mL to about 5.0 mL, about 3.5 mL to about 4.5 mL, about 3.5 mL to about 4 mL, about 4.0 mL to about 10 mL, about 4.0 mL to about 9.5 mL, about 4.0 mL to about 9.0 mL, about 4.0 mL to about 8.5 mL, about 4.0 mL to about 8.0 mL, about 4.0 mL to about 7.5 mL, about 4.0 mL to about 7.0 mL, about 4.0 mL to about 6.5 mL, about 4.0 mL to about 6 mL, about 4.0 mL to about 5.5 mL, about 4.0 mL to about 5.0 mL, about 4.0 mL to about 4.5 mL, about 4.5 mL to about 10 mL, about 4.5 mL to about 9.5 mL, about 4.5 mL to about 9.0 mL, about 4.5 mL to about 8.5 mL, about 4.5 mL to about 8.0 mL, about 4.5 mL to about 7.5 mL, about 4.5 mL to about 7.0 mL, about 4.5 mL to about 6.5 mL, about 4.5 mL to about 6 mL, about 4.5 mL to about 5.5 mL, about 4.5 mL to about 5.0 mL, about 5 mL to about 10 mL, about 5 mL to about 9.5 mL, about 5 mL to about 9.0 mL, about 5 mL to about 8.5 mL, about 5 mL to about 8.0 mL, about 5 mL to about 7.5 mL, about 5 mL to about 7.0 mL, about 5 mL to about 6.5 mL, about 5 mL to about 6 mL, about 5 mL to about 5.5 mL, about 5.5 mL to about 10 mL, about 5.5 mL to about 9.5 mL, about 5.5 mL to about 9.0 mL, about 5.5 mL to about 8.5 mL, about 5.5 mL to about 8.0 mL, about 5.5 mL to about 7.5 mL, about 5.5 mL to about 7.0 mL, about 5.5 mL to about 6.5 mL, about 5.5 mL to about 6 mL, about 6.0 mL to about 10 mL, about 6.0 mL to about 9.5 mL, about 6.0 mL to about 9.0 mL, about 6.0 mL to about 8.5 mL, about 6.0 mL to about 8.0 mL, about 6.0 mL to about 7.5 mL, about 6.0 mL to about 7.0 mL, about 6.0 mL to about 6.5 mL, about 6.5 mL to about 10 mL, about 6.5 mL to about 9.5 mL, about 6.5 mL to about 9.0 mL, about 6.5 mL to about 8.5 mL, about 6.5 mL to about 8.0 mL, about 6.5 mL to about 7.5 mL, about 6.5 mL to about 7.0 mL, about 7.0 mL to about 10 mL, about 7.0 mL to about 9.5 mL, about 7.0 mL to about 9.0 mL, about 7.0 mL to about 8.5 mL, about 7.0 mL to about 8.0 mL, about 7.0 mL to about 7.5 mL, about 7.5 mL to about 10 mL, about 7.5 mL to about 9.5 mL, about 7.5 mL to about 9.0 mL, about 7.5 mL to about 8.5 mL, about 7.5 mL to about 8.0 mL, about 8.0 mL to about 10 mL, about 8.0 mL to about 9.5 mL, about 8.0 mL to about 9.0 mL, about 8.0 mL to about 8.5 mL, about 8.5 mL to about 10 mL, about 8.5 mL to about 9.5 mL, about 8.5 mL to about 9.0 mL, about 9 mL to about 10 mL, about 9 mL to about 9.5 mL, or about 9.5 mL to about 10 mL. The composition may have a viscosity of less than or about 20 centipoise (cP). The composition may have a viscosity of less than or about 15 centipoise (cP). The composition may have a viscosity of less than or about 10 centipoise (cP). For instance, the composition has a viscosity of less than or about 20, 19, 18, 17, 16, 15, 14, 13, 12, 11, 10, 9, 8, 7, 6, 5, 4, 3, or 2 cP. The composition may have a viscosity of at least about 1, 2 or 3 cP. Further example viscosities include about 1 cP to about 2 cP, about 1 cP to about 3 cP, about 1 cP to about 4 cP, about 1 cP to about 5 cP, about 1 cP to about 6 cP, about 1 cP to about 7 cP, about 1 cP to about 8 cP, about 1 cP to about 9 cP, about 1 cP to about 10 cP, about 1 cP to about 11 cP, about 1 cP to about 12 cP, about 1 cP to about 13 cP, about 1 cP to about 14 cP, about 1 cP to about 15 cP, about 1 cP to about 16 cP, about 1 cP to about 17 cP, about 1 cP to about 18 cP, about 1 cP to about 19 cP, about 1 cP to about 20 cP, about 2 cP to about 5 cP, about 2 cP to about 6 cP, about 2 cP to about 7 cP, about 2 cP to about 8 cP, about 2 cP to about 9 cP, about 2 cP to about 10 cP, about 2 cP to about 11 cP, about 2 cP to about 12 cP, about 2 cP to about 13 cP, about 2 cP to about 14 cP, about 2 cP to about 15 cP, about 2 cP to about 16 cP, about 2 cP to about 17 cP, about 2 cP to about 18 cP, about 2 cP to about 19 cP, about 2 cP to about 20 cP, about 3 cP to about 5 cP, about 3 cP to about 6 cP, about 3 cP to about 7 cP, about 3 cP to about 8 cP, about 3 cP to about 9 cP, about 3 cP to about 10 cP, about 3 cP to about 11 cP, about 3 cP to about 12 cP, about 3 cP to about 13 cP, about 3 cP to about 14 cP, about 3 cP to about 15 cP, about 3 cP to about 16 cP, about 3 cP to about 17 cP, about 3 cP to about 18 cP, about 3 cP to about 19 cP, about cP to about 20 cP, about 4 cP to about 5 cP, about 4 cP to about 6 cP, about 4 cP to about 7 cP, about 4 cP to about 8 cP, about 4 cP to about 9 cP, about 4 cP to about 10 cP. about 4 cP to about 11 cP, about 4 cP to about 12 cP, about 4 cP to about 13 cP, about 4 cP to about 14 cP, about 4 cP to about 15 cP, about 4 cP to about 16 cP, about 4 cP to about 17 cP, about 4 cP to about 18 cP, about 4 cP to about 19 cP, about 4 cP to about 20 cP, about 5 cP to about 10 cP, about 5 cP to about 11 cP, about 5 cP to about 12 cP, about 5 cP to about 13 cP, about 5 cP to about 14 cP, about 5 cP to about 15 cP, about 5 cP to about 16 cP, about 5 cP to about 17 cP, about 5 cP to about 18 cP, about 5 cP to about 19 cP, about 5 cP to about 20 cP, about 6 cP to about 10 cP, about 6 cP to about 11 cP, about 6 cP to about 12 cP, about 6 cP to about 13 cP, about 6 cP to about 14 cP, about 6 cP to about 15 cP, about 6 cP to about 16 cP, about 6 cP to about 17 cP, about 6 cP to about 18 cP, about 6 cP to about 19 cP, about 6 cP to about 20 cP, about 7 cP to about 10 cP, about 7 cP to about 11 cP, about 7 cP to about 12 cP, about 7 cP to about 13 cP, about 7 cP to about 14 cP, about 7 cP to about 15 cP, about 7 cP to about 16 cP, about 7 cP to about 17 cP, about 7 cP to about 18 cP, about 7 cP to about 19 cP, about 7 cP to about 20 cP, about 8 cP to about 10 cP, about 8 cP to about 11 cP, about 8 cP to about 12 cP, about 8 cP to about 13 cP, about 8 cP to about 14 cP, about 8 cP to about 15 cP, about 8 cP to about 16 cP, about 8 cP to about 17 cP, about 8 cP to about 18 cP, about 8 cP to about 19 cP, or about 8 cP to about 20 cP. In some embodiments, the concentration of anti-TL1A or anti-IL23 is about or greater than about 55, 60, 65, 70, 75, 80, 85, 90, 95, 100, 105, 110, 115, 120, 125, 130, 135, 140, 145, 155, 160, 165, 170, 175, 180, 185, 190, 195, 200, 205, 210, 215, 220, 225, 230, 235, 240, 245, or 250 mg/mL.

In certain embodiments, provided herein is a pharmaceutical composition comprising a therapeutically effective dose of a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2), wherein the pharmaceutical composition has a viscosity of less than about 20 cP, 15 cP, or 10 cP. The composition may have a viscosity of less than or about 20 cP. The composition may have a viscosity of less than or about 15 cP. The composition may have a viscosity of less than or about 10 cP. For instance, the composition has a viscosity of less than or about 20, 19, 18, 17, 16, 15, 14, 13, 12, 11, 10, 9, 8, 7, 6, 5, 4, 3, or 2 cP. The composition may have a viscosity of at least about 1, 2 or 3 cP. Further example viscosities include about 1 cP to about 2 cP, about 1 cP to about 3 cP, about 1 cP to about 4 cP, about 1 cP to about 5 cP, about 1 cP to about 6 cP, about 1 cP to about 7 cP, about 1 cP to about 8 cP, about 1 cP to about 9 cP, about 1 cP to about 10 cP, about 1 cP to about 11 cP, about 1 cP to about 12 cP, about 1 cP to about 13 cP, about 1 cP to about 14 cP, about 1 cP to about 15 cP, about 1 cP to about 16 cP, about 1 cP to about 17 cP, about 1 cP to about 18 cP, about 1 cP to about 19 cP, about 1 cP to about 20 cP, about 2 cP to about 5 cP, about 2 cP to about 6 cP, about 2 cP to about 7 cP, about 2 cP to about 8 cP, about 2 cP to about 9 cP, about 2 cP to about 10 cP, about 2 cP to about 11 cP, about 2 cP to about 12 cP, about 2 cP to about 13 cP, about 2 cP to about 14 cP, about 2 cP to about 15 cP, about 2 cP to about 16 cP, about 2 cP to about 17 cP, about 2 cP to about 18 cP, about 2 cP to about 19 cP, about 2 cP to about 20 cP, about 3 cP to about 5 cP, about 3 cP to about 6 cP, about 3 cP to about 7 cP, about 3 cP to about 8 cP, about 3 cP to about 9 cP, about 3 cP to about 10 cP, about 3 cP to about 11 cP, about 3 cP to about 12 cP, about 3 cP to about 13 cP, about 3 cP to about 14 cP, about 3 cP to about 15 cP, about 3 cP to about 16 cP, about 3 cP to about 17 cP, about 3 cP to about 18 cP, about 3 cP to about 19 cP, about cP to about 20 cP, about 4 cP to about 5 cP, about 4 cP to about 6 cP, about 4 cP to about 7 cP, about 4 cP to about 8 cP, about 4 cP to about 9 cP, about 4 cP to about 10 cP. about 4 cP to about 11 cP, about 4 cP to about 12 cP, about 4 cP to about 13 cP, about 4 cP to about 14 cP, about 4 cP to about 15 cP, about 4 cP to about 16 cP, about 4 cP to about 17 cP, about 4 cP to about 18 cP, about 4 cP to about 19 cP, about 4 cP to about 20 cP, about 5 cP to about 10 cP, about 5 cP to about 11 cP, about 5 cP to about 12 cP, about 5 cP to about 13 cP, about 5 cP to about 14 cP, about 5 cP to about 15 cP, about 5 cP to about 16 cP, about 5 cP to about 17 cP, about 5 cP to about 18 cP, about 5 cP to about 19 cP, about 5 cP to about 20 cP, about 6 cP to about 10 cP, about 6 cP to about 11 cP, about 6 cP to about 12 cP, about 6 cP to about 13 cP, about 6 cP to about 14 cP, about 6 cP to about 15 cP, about 6 cP to about 16 cP, about 6 cP to about 17 cP, about 6 cP to about 18 cP, about 6 cP to about 19 cP, about 6 cP to about 20 cP, about 7 cP to about 10 cP, about 7 cP to about 11 cP, about 7 cP to about 12 cP, about 7 cP to about 13 cP, about 7 cP to about 14 cP, about 7 cP to about 15 cP, about 7 cP to about 16 cP, about 7 cP to about 17 cP, about 7 cP to about 18 cP, about 7 cP to about 19 cP, about 7 cP to about 20 cP, about 8 cP to about 10 cP, about 8 cP to about 11 cP, about 8 cP to about 12 cP, about 8 cP to about 13 cP, about 8 cP to about 14 cP, about 8 cP to about 15 cP, about 8 cP to about 16 cP, about 8 cP to about 17 cP, about 8 cP to about 18 cP, about 8 cP to about 19 cP, or about 8 cP to about 20 cP. In some embodiments, the therapeutically effective dose is at least about 150 mg TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2). In some cases, the therapeutically effective dose is about or at least about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of anti-TL1A or anti-IL23. In some cases, the therapeutically effective dose is about 150 mg to about 2000 mg, about 150 mg to about 1750 mg, about 150 mg to about 1500 mg, about 150 mg to about 1250 mg, about 150 mg to about 1000 mg, about 150 mg to about 750 mg, about 150 mg to about 500 mg, about 150 mg to about 450 mg, about 150 mg to about 400 mg, about 150 mg to about 350 mg, about 150 mg to about 300 mg, about 150 mg to about 250 mg, or about 150 mg to about 200 mg anti-TL1A or anti-IL23. The total volume of the composition may be less than or equal to about 2 mL. The total volume of the composition may be less than or equal to about 2.5 mL. The total volume may be less than about or equal to about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, or 0.8 mL. The total volume may be at least about 0.5 mL. The total volume may be about 0.5 mL to about 3 mL, about 0.5 mL to about 2.9 mL, about 0.5 mL to about 2.8 mL, about 0.5 mL to about 2.7 mL, about 0.5 mL to about 2.6 mL, about 0.5 mL to about 2.5 mL, about 0.5 mL to about 2.4 mL, about 0.5 mL to about 2.3 mL, about 0.5 mL to about 2.2 mL, about 0.5 mL to about 2.1 mL, about 0.5 mL to about 2 mL, 0.5 mL to about 1.9 mL, 0.5 mL to about 1.8 mL, 0.5 mL to about 1.7 mL, 0.5 mL to about 1.6 mL, about 0.5 mL to about 1.5 mL, about 0.5 mL to about 1.4 mL, about 0.5 mL to about 1.3 mL, about 0.5 mL to about 1.2 mL, about 0.5 mL to about 1.1 mL, about 0.5 mL to about 1.0 mL, about 0.5 mL to about 0.9 mL, about 0.5 mL to about 0.8 mL, about 0.6 mL to about 3 mL, about 0.6 mL to about 2.9 mL, about 0.6 mL to about 2.8 mL, about 0.6 mL to about 2.7 mL, about 0.6 mL to about 2.6 mL, about 0.6 mL to about 2.5 mL, about 0.6 mL to about 2.4 mL, about 0.6 mL to about 2.3 mL, about 0.6 mL to about 2.2 mL, about 0.6 mL to about 2.1 mL, about 0.6 mL to about 2.0 mL, about 0.6 mL to about 1.9 mL, about 0.6 mL to about 1.8 mL, about 0.6 mL to about 1.7 mL, about 0.6 mL to about 1.6 mL, about 0.6 mL to about 1.5 mL, about 0.6 mL to about 1.4 mL, about 0.6 mL to about 1.3 mL, about 0.6 mL to about 1.2 mL, about 0.6 mL to about 1.1 mL, about 0.6 mL to about 1.0 mL, about 0.6 mL to about 0.9 mL, about 0.6 mL to about 0.8 mL, about 0.7 mL to about 3 mL, about 0.7 mL to about 2.9 mL, about 0.7 mL to about 2.8 mL, about 0.7 mL to about 2.7 mL, about 0.7 mL to about 2.6 mL, about 0.7 mL to about 2.5 mL, about 0.7 mL to about 2.4 mL, about 0.7 mL to about 2.3 mL, about 0.7 mL to about 2.2 mL, about 0.7 mL to about 2.1 mL, about 0.7 mL to about 2.0 mL, about 0.7 mL to about 1.9 mL, about 0.7 mL to about 1.8 mL, about 0.7 mL to about 1.7 mL, about 0.7 mL to about 1.6 mL, about 0.7 mL to about 1.5 mL, about 0.7 mL to about 1.4 mL, about 0.7 mL to about 1.3 mL, about 0.7 mL to about 1.2 mL, about 0.7 mL to about 1.1 mL, about 0.7 mL to about 1.0 mL, about 0.7 mL to about 0.9 mL, about 0.7 mL to about 0.8 mL, about 3 mL to about 10 mL, about 3 mL to about 9.5 mL, about 3 mL to about 9.0 mL, about 3 mL to about 8.5 mL, about 3 mL to about 8.0 mL, about 3 mL to about 7.5 mL, about 3 mL to about 7.0 mL, about 3 mL to about 6.5 mL, about 3 mL to about 6 mL, about 3 mL to about 5.5 mL, about 3 mL to about 5.0 mL, about 3 mL to about 4.5 mL, about 3 mL to about 4 mL, about 3 mL to about 3.5 mL, about 3.5 mL to about 10 mL, about 3.5 mL to about 9.5 mL, about 3.5 mL to about 9.0 mL, about 3.5 mL to about 8.5 mL, about 3.5 mL to about 8.0 mL, about 3.5 mL to about 7.5 mL, about 3.5 mL to about 7.0 mL, about 3.5 mL to about 6.5 mL, about 3.5 mL to about 6 mL, about 3.5 mL to about 5.5 mL, about 3.5 mL to about 5.0 mL, about 3.5 mL to about 4.5 mL, about 3.5 mL to about 4 mL, about 4.0 mL to about 10 mL, about 4.0 mL to about 9.5 mL, about 4.0 mL to about 9.0 mL, about 4.0 mL to about 8.5 mL, about 4.0 mL to about 8.0 mL, about 4.0 mL to about 7.5 mL, about 4.0 mL to about 7.0 mL, about 4.0 mL to about 6.5 mL, about 4.0 mL to about 6 mL, about 4.0 mL to about 5.5 mL, about 4.0 mL to about 5.0 mL, about 4.0 mL to about 4.5 mL, about 4.5 mL to about 10 mL, about 4.5 mL to about 9.5 mL, about 4.5 mL to about 9.0 mL, about 4.5 mL to about 8.5 mL, about 4.5 mL to about 8.0 mL, about 4.5 mL to about 7.5 mL, about 4.5 mL to about 7.0 mL, about 4.5 mL to about 6.5 mL, about 4.5 mL to about 6 mL, about 4.5 mL to about 5.5 mL, about 4.5 mL to about 5.0 mL, about 5 mL to about 10 mL, about 5 mL to about 9.5 mL, about 5 mL to about 9.0 mL, about 5 mL to about 8.5 mL, about 5 mL to about 8.0 mL, about 5 mL to about 7.5 mL, about 5 mL to about 7.0 mL, about 5 mL to about 6.5 mL, about 5 mL to about 6 mL, about 5 mL to about 5.5 mL, about 5.5 mL to about 10 mL, about 5.5 mL to about 9.5 mL, about 5.5 mL to about 9.0 mL, about 5.5 mL to about 8.5 mL, about 5.5 mL to about 8.0 mL, about 5.5 mL to about 7.5 mL, about 5.5 mL to about 7.0 mL, about 5.5 mL to about 6.5 mL, about 5.5 mL to about 6 mL, about 6.0 mL to about 10 mL, about 6.0 mL to about 9.5 mL, about 6.0 mL to about 9.0 mL, about 6.0 mL to about 8.5 mL, about 6.0 mL to about 8.0 mL, about 6.0 mL to about 7.5 mL, about 6.0 mL to about 7.0 mL, about 6.0 mL to about 6.5 mL, about 6.5 mL to about 10 mL, about 6.5 mL to about 9.5 mL, about 6.5 mL to about 9.0 mL, about 6.5 mL to about 8.5 mL, about 6.5 mL to about 8.0 mL, about 6.5 mL to about 7.5 mL, about 6.5 mL to about 7.0 mL, about 7.0 mL to about 10 mL, about 7.0 mL to about 9.5 mL, about 7.0 mL to about 9.0 mL, about 7.0 mL to about 8.5 mL, about 7.0 mL to about 8.0 mL, about 7.0 mL to about 7.5 mL, about 7.5 mL to about 10 mL, about 7.5 mL to about 9.5 mL, about 7.5 mL to about 9.0 mL, about 7.5 mL to about 8.5 mL, about 7.5 mL to about 8.0 mL, about 8.0 mL to about 10 mL, about 8.0 mL to about 9.5 mL, about 8.0 mL to about 9.0 mL, about 8.0 mL to about 8.5 mL, about 8.5 mL to about 10 mL, about 8.5 mL to about 9.5 mL, about 8.5 mL to about 9.0 mL, about 9 mL to about 10 mL, about 9 mL to about 9.5 mL, or about 9.5 mL to about 10 mL. In some embodiments, the concentration of anti-TL1A or anti-IL23 is about or greater than about 55, 60, 65, 70, 75, 80, 85, 90, 95, 100, 105, 110, 115, 120, 125, 130, 135, 140, 145, 155, 160, 165, 170, 175, 180, 185, 190, 195, 200, 205, 210, 215, 220, 225, 230, 235, 240, 245, or 250 mg/mL.

In certain embodiments, provided herein is a pharmaceutical composition comprising a therapeutically effective dose of a TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) having a percentage aggregation of the TL1A inhibitor or IL23 inhibitor as measured by size exclusion chromatography of less than about 5% of the respective total TL1A inhibitor or IL23 inhibitor in the composition. In some embodiments, the percentage aggregation of the TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2) as measured by size exclusion chromatography is less than about 4.5%, 4%, 3.5%, 3%, 2.5%, 2%, 1.5%, 1%, 0.9%, 0.8%, 0.7%, 0.6%, 0.5%, 0.4%, 0.3%, 0.2%, 0.1% of the composition volume. In some embodiments, the therapeutically effective dose is at least about 150 mg TL1A inhibitor (such as an anti-TL1A antibody or antigen-binding fragment thereof or soluble proteins of Section 4.3.1(c)) or an IL23 inhibitor (such as the anti-IL23 antibody or antigen-binding fragment thereof of Section 4.3.2). In some cases, the therapeutically effective dose is about or at least about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of anti-TL1A or anti-IL23. In some cases, the therapeutically effective dose is about 150 mg to about 2000 mg, about 150 mg to about 1750 mg, about 150 mg to about 1500 mg, about 150 mg to about 1250 mg, about 150 mg to about 1000 mg, about 150 mg to about 750 mg, about 150 mg to about 500 mg, about 150 mg to about 450 mg, about 150 mg to about 400 mg, about 150 mg to about 350 mg, about 150 mg to about 300 mg, about 150 mg to about 250 mg, or about 150 mg to about 200 mg anti-TL1A or anti-IL23. The total volume of the composition may be less than or equal to about 2 mL. The total volume of the composition may be less than or equal to about 2.5 mL. The total volume may be less than about or equal to about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, or 0.8 mL. The total volume may be at least about 0.5 mL. The total volume may be about 0.5 mL to about 3 mL, about 0.5 mL to about 2.9 mL, about 0.5 mL to about 2.8 mL, about 0.5 mL to about 2.7 mL, about 0.5 mL to about 2.6 mL, about 0.5 mL to about 2.5 mL, about 0.5 mL to about 2.4 mL, about 0.5 mL to about 2.3 mL, about 0.5 mL to about 2.2 mL, about 0.5 mL to about 2.1 mL, about 0.5 mL to about 2 mL, 0.5 mL to about 1.9 mL, 0.5 mL to about 1.8 mL, 0.5 mL to about 1.7 mL, 0.5 mL to about 1.6 mL, about 0.5 mL to about 1.5 mL, about 0.5 mL to about 1.4 mL, about 0.5 mL to about 1.3 mL, about 0.5 mL to about 1.2 mL, about 0.5 mL to about 1.1 mL, about 0.5 mL to about 1.0 mL, about 0.5 mL to about 0.9 mL, about 0.5 mL to about 0.8 mL, about 0.6 mL to about 3 mL, about 0.6 mL to about 2.9 mL, about 0.6 mL to about 2.8 mL, about 0.6 mL to about 2.7 mL, about 0.6 mL to about 2.6 mL, about 0.6 mL to about 2.5 mL, about 0.6 mL to about 2.4 mL, about 0.6 mL to about 2.3 mL, about 0.6 mL to about 2.2 mL, about 0.6 mL to about 2.1 mL, about 0.6 mL to about 2.0 mL, about 0.6 mL to about 1.9 mL, about 0.6 mL to about 1.8 mL, about 0.6 mL to about 1.7 mL, about 0.6 mL to about 1.6 mL, about 0.6 mL to about 1.5 mL, about 0.6 mL to about 1.4 mL, about 0.6 mL to about 1.3 mL, about 0.6 mL to about 1.2 mL, about 0.6 mL to about 1.1 mL, about 0.6 mL to about 1.0 mL, about 0.6 mL to about 0.9 mL, about 0.6 mL to about 0.8 mL, about 0.7 mL to about 3 mL, about 0.7 mL to about 2.9 mL, about 0.7 mL to about 2.8 mL, about 0.7 mL to about 2.7 mL, about 0.7 mL to about 2.6 mL, about 0.7 mL to about 2.5 mL, about 0.7 mL to about 2.4 mL, about 0.7 mL to about 2.3 mL, about 0.7 mL to about 2.2 mL, about 0.7 mL to about 2.1 mL, about 0.7 mL to about 2.0 mL, about 0.7 mL to about 1.9 mL, about 0.7 mL to about 1.8 mL, about 0.7 mL to about 1.7 mL, about 0.7 mL to about 1.6 mL, about 0.7 mL to about 1.5 mL, about 0.7 mL to about 1.4 mL, about 0.7 mL to about 1.3 mL, about 0.7 mL to about 1.2 mL, about 0.7 mL to about 1.1 mL, about 0.7 mL to about 1.0 mL, about 0.7 mL to about 0.9 mL, about 0.7 mL to about 0.8 mL, about 3 mL to about 10 mL, about 3 mL to about 9.5 mL, about 3 mL to about 9.0 mL, about 3 mL to about 8.5 mL, about 3 mL to about 8.0 mL, about 3 mL to about 7.5 mL, about 3 mL to about 7.0 mL, about 3 mL to about 6.5 mL, about 3 mL to about 6 mL, about 3 mL to about 5.5 mL, about 3 mL to about 5.0 mL, about 3 mL to about 4.5 mL, about 3 mL to about 4 mL, about 3 mL to about 3.5 mL, about 3.5 mL to about 10 mL, about 3.5 mL to about 9.5 mL, about 3.5 mL to about 9.0 mL, about 3.5 mL to about 8.5 mL, about 3.5 mL to about 8.0 mL, about 3.5 mL to about 7.5 mL, about 3.5 mL to about 7.0 mL, about 3.5 mL to about 6.5 mL, about 3.5 mL to about 6 mL, about 3.5 mL to about 5.5 mL, about 3.5 mL to about 5.0 mL, about 3.5 mL to about 4.5 mL, about 3.5 mL to about 4 mL, about 4.0 mL to about 10 mL, about 4.0 mL to about 9.5 mL, about 4.0 mL to about 9.0 mL, about 4.0 mL to about 8.5 mL, about 4.0 mL to about 8.0 mL, about 4.0 mL to about 7.5 mL, about 4.0 mL to about 7.0 mL, about 4.0 mL to about 6.5 mL, about 4.0 mL to about 6 mL, about 4.0 mL to about 5.5 mL, about 4.0 mL to about 5.0 mL, about 4.0 mL to about 4.5 mL, about 4.5 mL to about 10 mL, about 4.5 mL to about 9.5 mL, about 4.5 mL to about 9.0 mL, about 4.5 mL to about 8.5 mL, about 4.5 mL to about 8.0 mL, about 4.5 mL to about 7.5 mL, about 4.5 mL to about 7.0 mL, about 4.5 mL to about 6.5 mL, about 4.5 mL to about 6 mL, about 4.5 mL to about 5.5 mL, about 4.5 mL to about 5.0 mL, about 5 mL to about 10 mL, about 5 mL to about 9.5 mL, about 5 mL to about 9.0 mL, about 5 mL to about 8.5 mL, about 5 mL to about 8.0 mL, about 5 mL to about 7.5 mL, about 5 mL to about 7.0 mL, about 5 mL to about 6.5 mL, about 5 mL to about 6 mL, about 5 mL to about 5.5 mL, about 5.5 mL to about 10 mL, about 5.5 mL to about 9.5 mL, about 5.5 mL to about 9.0 mL, about 5.5 mL to about 8.5 mL, about 5.5 mL to about 8.0 mL, about 5.5 mL to about 7.5 mL, about 5.5 mL to about 7.0 mL, about 5.5 mL to about 6.5 mL, about 5.5 mL to about 6 mL, about 6.0 mL to about 10 mL, about 6.0 mL to about 9.5 mL, about 6.0 mL to about 9.0 mL, about 6.0 mL to about 8.5 mL, about 6.0 mL to about 8.0 mL, about 6.0 mL to about 7.5 mL, about 6.0 mL to about 7.0 mL, about 6.0 mL to about 6.5 mL, about 6.5 mL to about 10 mL, about 6.5 mL to about 9.5 mL, about 6.5 mL to about 9.0 mL, about 6.5 mL to about 8.5 mL, about 6.5 mL to about 8.0 mL, about 6.5 mL to about 7.5 mL, about 6.5 mL to about 7.0 mL, about 7.0 mL to about 10 mL, about 7.0 mL to about 9.5 mL, about 7.0 mL to about 9.0 mL, about 7.0 mL to about 8.5 mL, about 7.0 mL to about 8.0 mL, about 7.0 mL to about 7.5 mL, about 7.5 mL to about 10 mL, about 7.5 mL to about 9.5 mL, about 7.5 mL to about 9.0 mL, about 7.5 mL to about 8.5 mL, about 7.5 mL to about 8.0 mL, about 8.0 mL to about 10 mL, about 8.0 mL to about 9.5 mL, about 8.0 mL to about 9.0 mL, about 8.0 mL to about 8.5 mL, about 8.5 mL to about 10 mL, about 8.5 mL to about 9.5 mL, about 8.5 mL to about 9.0 mL, about 9 mL to about 10 mL, about 9 mL to about 9.5 mL, or about 9.5 mL to about 10 mL. In some embodiments, the concentration of anti-TL1A or anti-IL23 is about or greater than about 55, 60, 65, 70, 75, 80, 85, 90, 95, 100, 105, 110, 115, 120, 125, 130, 135, 140, 145, 155, 160, 165, 170, 175, 180, 185, 190, 195, 200, 205, 210, 215, 220, 225, 230, 235, 240, 245, or 250 mg/mL.

In certain embodiments, the pharmaceutical composition has a volume suitable for injection, such as via subcutaneous administration. In some embodiments, the total volume of the composition may be less than or equal to about 2.5 mL. In some embodiments, the total volume of the composition is less than about 2 mL, less than about or equal to about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, or 0.8 mL. Antibody therapeutics suitable for injection and/or administration are important to realizing full therapeutic potential. However, administration is generally restricted by volume, for instance, when the therapeutic is delivered subcutaneously. This, in turn, elucidates the importance developing of high concentration antibody formulations of greater than, for example in some cases, 100 mg/ml. Problems associated with antibody development include high solution viscosity and opalescence, which are commonly encountered during the development of high-concentration (e.g., greater than 100 mg/ml). Both viscosity and opalescence impact antibody developability broadly, affecting manufacturability, stability, and delivery. High solution viscosities (e.g., greater than 30 mPa-s) cause limiting back-pressures in ultrafiltration/diafiltration during the antibody concentration unit operation. Similarly, viscous antibody solutions also result in forbidding or incompatible injection forces when administering via injection, including via patient friendly autoinjectors. In effect, solution viscosity can be a determining factor for the maximum antibody dose possible via injection. Solution opalescence in therapeutic antibodies can be equally problematic as opalescence can indicate predisposition for liquid-liquid phase separation, precipitation, or aggregation. Further difficulty may occur with blinding of subcutaneous placebo.

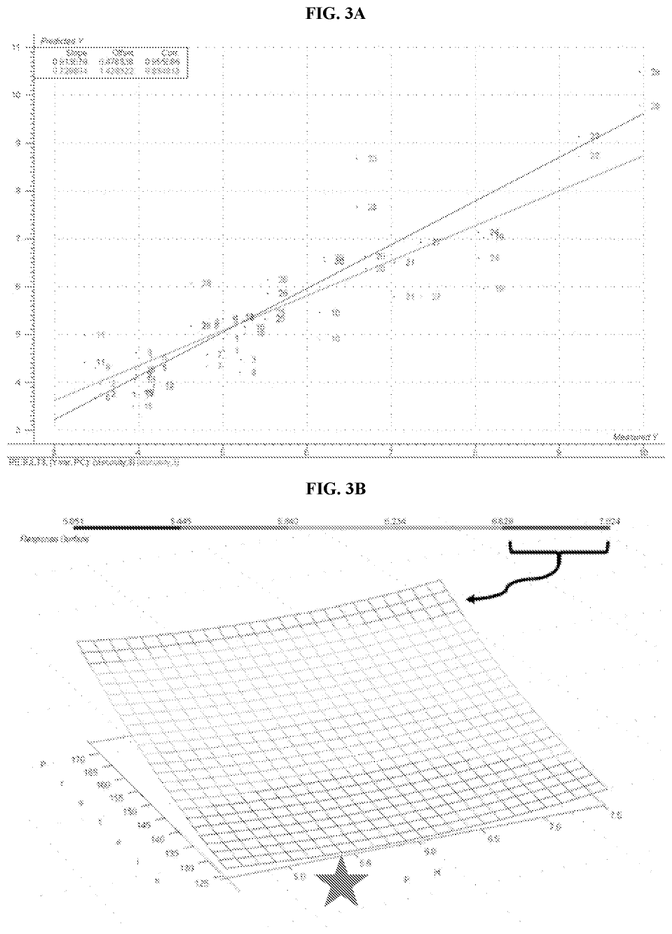

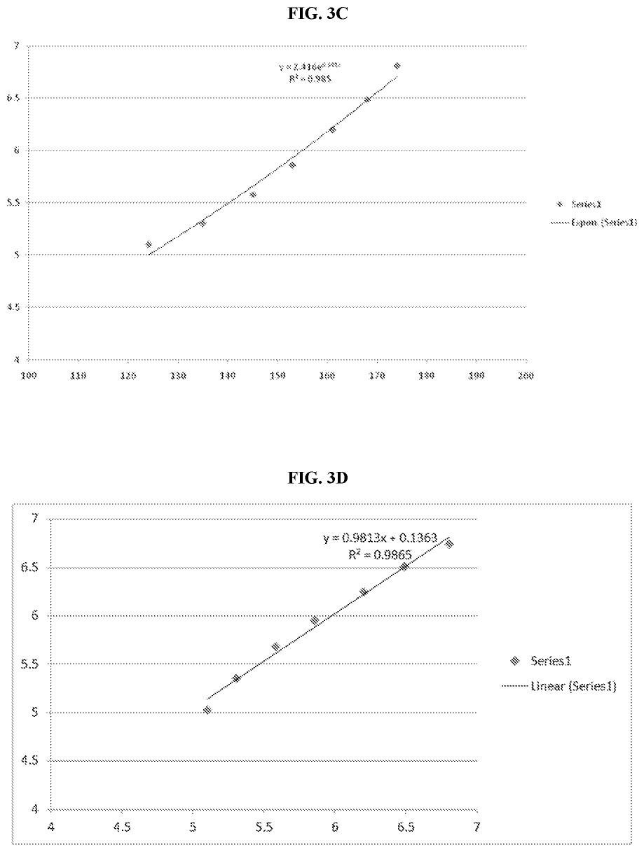

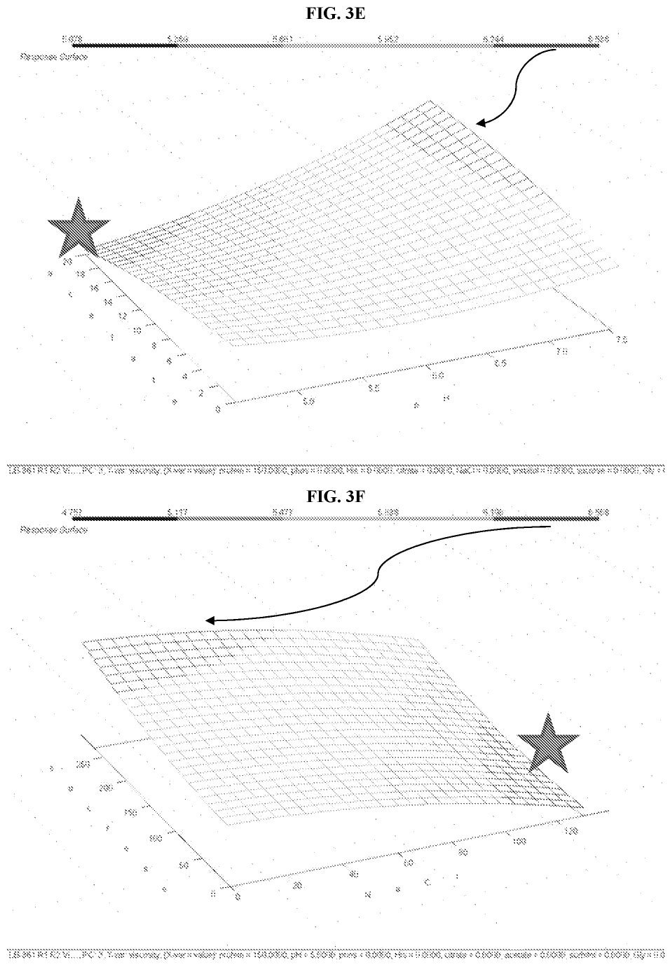

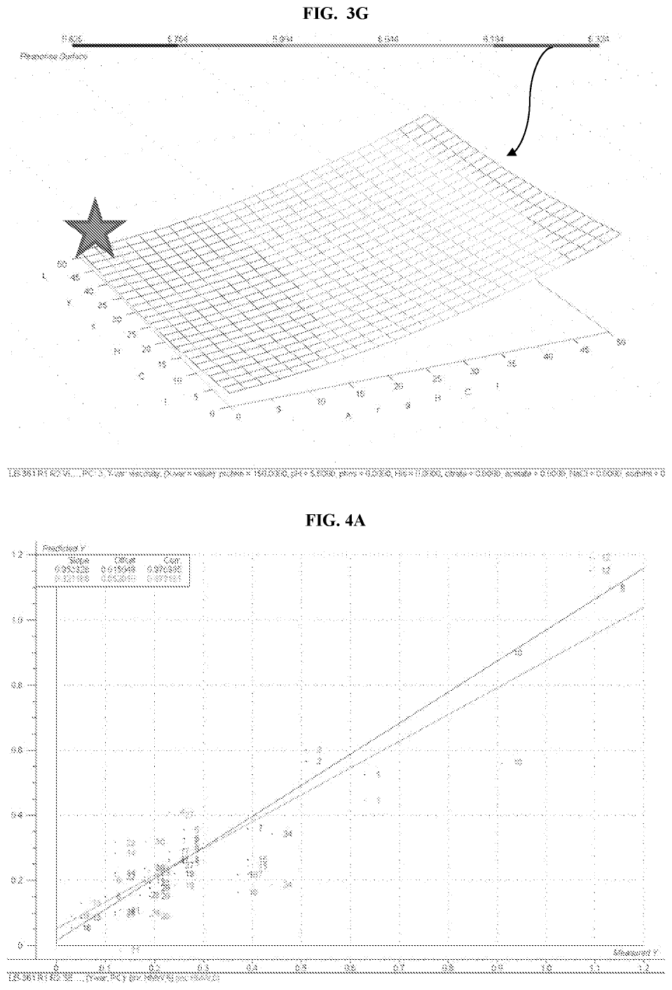

The anti-TL1A antibodies provided herein demonstrate advantageous viscosity and aggregation properties at high antibody concentrations (e.g., greater than about 100, 125, 150, 160, 170, 180, 190, or 200 mg/mL). Notably, anti-TL1A antibodies provided herein are characterized by low viscosity (e.g., less than 20 mPa-s) and low aggregation (e.g., less than 5% high molecular weight species) at high concentrations (FIGS. 3A-3C).

For example, in some embodiments, the anti-T1LA antibody is characterized by a viscosity less than about 30, 20, 15, or 10 mPa-s at a concentration greater than about 100 mg/mL, e.g., about 150 mg/mL to about 300 mg/mL, about 150 mg/mL to about 200 mg/mL, about 150 mg/mL to about 225 mg/mL, or about 150 mg/mL to about 250 mg/mL. In some cases, the antibody comprises a HCDR1 comprising SEQ ID NO: 1, a HCDR2 comprising SEQ ID NO: 2, a HCDR3 comprising SEQ ID NO: 6, a LCDR1 comprising SEQ ID NO: 10, a LCDR2 comprising SEQ ID NO: 11, and a LCDR3 comprising SEQ ID NO: 12, and/or having a heavy chain variable region comprising SEQ ID NO: 104 and a light chain variable region comprising SEQ ID NO: 201. In some cases, the anti-TL1A antibody comprises a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework. For instance, the composition has a viscosity of less than or about 20, 19, 18, 17, 16, 15, 14, 13, 12, 11, 10, 9, 8, 7, 6, 5, 4, 3, or 2 cP. The composition may have a viscosity of at least about 1, 2 or 3 cP. Further example viscosities include about 1 cP to about 5 cP, about 1 cP to about 6 cP, about 1 cP to about 7 cP, about 1 cP to about 8 cP, about 1 cP to about 9 cP, about 1 cP to about 10 cP, about 1 cP to about 11 cP, about 1 cP to about 12 cP, about 1 cP to about 13 cP, about 1 cP to about 14 cP, about 1 cP to about 15 cP, about 1 cP to about 16 cP, about 1 cP to about 17 cP, about 1 cP to about 18 cP, about 1 cP to about 19 cP, about 1 cP to about 20 cP, about 2 cP to about 5 cP, about 2 cP to about 6 cP, about 2 cP to about 7 cP, about 2 cP to about 8 cP, about 2 cP to about 9 cP, about 2 cP to about 10 cP, about 2 cP to about 11 cP, about 2 cP to about 12 cP, about 2 cP to about 13 cP, about 2 cP to about 14 cP, about 2 cP to about 15 cP, about 2 cP to about 16 cP, about 2 cP to about 17 cP, about 2 cP to about 18 cP, about 2 cP to about 19 cP, about 2 cP to about 20 cP, about 3 cP to about 5 cP, about 3 cP to about 6 cP, about 3 cP to about 7 cP, about 3 cP to about 8 cP, about 3 cP to about 9 cP, about 3 cP to about 10 cP, about 3 cP to about 11 cP, about 3 cP to about 12 cP, about 3 cP to about 13 cP, about 3 cP to about 14 cP, about 3 cP to about 15 cP, about 3 cP to about 16 cP, about 3 cP to about 17 cP, about 3 cP to about 18 cP, about 3 cP to about 19 cP, about cP to about 20 cP, about 4 cP to about 5 cP, about 4 cP to about 6 cP, about 4 cP to about 7 cP, about 4 cP to about 8 cP, about 4 cP to about 9 cP, about 4 cP to about 10 cP. about 4 cP to about 11 cP, about 4 cP to about 12 cP, about 4 cP to about 13 cP, about 4 cP to about 14 cP, about 4 cP to about 15 cP, about 4 cP to about 16 cP, about 4 cP to about 17 cP, about 4 cP to about 18 cP, about 4 cP to about 19 cP, about 4 cP to about 20 cP, about 5 cP to about 10 cP, about 5 cP to about 11 cP, about 5 cP to about 12 cP, about 5 cP to about 13 cP, about 5 cP to about 14 cP, about 5 cP to about 15 cP, about 5 cP to about 16 cP, about 5 cP to about 17 cP, about 5 cP to about 18 cP, about 5 cP to about 19 cP, about 5 cP to about 20 cP, about 6 cP to about 10 cP, about 6 cP to about 11 cP, about 6 cP to about 12 cP, about 6 cP to about 13 cP, about 6 cP to about 14 cP, about 6 cP to about 15 cP, about 6 cP to about 16 cP, about 6 cP to about 17 cP, about 6 cP to about 18 cP, about 6 cP to about 19 cP, about 6 cP to about 20 cP, about 7 cP to about 10 cP, about 7 cP to about 11 cP, about 7 cP to about 12 cP, about 7 cP to about 13 cP, about 7 cP to about 14 cP, about 7 cP to about 15 cP, about 7 cP to about 16 cP, about 7 cP to about 17 cP, about 7 cP to about 18 cP, about 7 cP to about 19 cP, about 7 cP to about 20 cP, about 8 cP to about 10 cP, about 8 cP to about 11 cP, about 8 cP to about 12 cP, about 8 cP to about 13 cP, about 8 cP to about 14 cP, about 8 cP to about 15 cP, about 8 cP to about 16 cP, about 8 cP to about 17 cP, about 8 cP to about 18 cP, about 8 cP to about 19 cP, or about 8 cP to about 20 cP, at a concentration of about 150 mg/ml to about 300 mg/ml, about 150 mg/ml to about 200 mg/ml, or about 150, 155, 160, 165, 170, 175, 180, 185, 190, 195, 200, 205, 210, 215, 220, or 225 mg/ml. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 150 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 160 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 170 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 180 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 190 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 200 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 210 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 220 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 230 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 240 mg/mL. In some embodiments, the anti-T1LA antibody is characterized by a viscosity about or less than about 20, 19, 18, 17, 16, 15, 14, 13, 12, 11, 10, 9, 8, 7, 6, or 5 mPa-s at a concentration greater than or about 250 mg/mL. In some embodiments, the anti-T1LA antibody is characterized by a viscosity about or less than about 20, 19, 18 17, 16, 15, 14, 13, 12, 11, 10, 9, 8, 7, 6, or 5 mPa-s at a concentration of about 150 mg/ml to about 250 mg/ml. In some embodiments, less than about 20 mPa-s includes from about 2 to about 20 mPa-s, from about 2 to about 19 mPa-s, from about 2 to about 18 mPa-s, from about 2 to about 17 mPa-s, from about 2 to about 16 mPa-s, from about 2 to about 15 mPa-s, from about 2 to about 14 mPa-s, from about 2 to about 13 mPa-s, from about 2 to about 12 mPa-s, from about 2 to about 11 mPa-s, from about 2 to about 10 mPa-s, from about 2 to about 9 mPa-s, from about 2 to about 8 mPa-s, from about 2 to about 7 mPa-s, from about 2 to about 6 mPa-s, from about 2 to about 5 mPa-s, from about 3 to about 20 mPa-s, from about 3 to about 19 mPa-s, from about 3 to about 18 mPa-s, from about 3 to about 17 mPa-s, from about 3 to about 16 mPa-s, from about 3 to about 15 mPa-s, from about 3 to about 14 mPa-s, from about 3 to about 13 mPa-s, from about 3 to about 12 mPa-s, from about 3 to about 11 mPa-s, from about 3 to about 10 mPa-s, from about 3 to about 9 mPa-s, from about 3 to about 8 mPa-s, from about 3 to about 7 mPa-s, from about 3 to about 6 mPa-s, from about 3 to about 5 mPa-s, from about 4 to about 20 mPa-s, from about 4 to about 19 mPa-s, from about 4 to about 18 mPa-s, from about 4 to about 17 mPa-s, from about 4 to about 16 mPa-s, from about 4 to about 15 mPa-s, from about 4 to about 14 mPa-s, from about 4 to about 13 mPa-s, from about 4 to about 12 mPa-s, from about 4 to about 11 mPa-s, from about 4 to about 10 mPa-s, from about 4 to about 9 mPa-s, from about 4 to about 8 mPa-s, from about 4 to about 7 mPa-s, from about 4 to about 6 mPa-s, from about 4 to about 5 mPa-s, from about 5 to about 20 mPa-s, from about 5 to about 19 mPa-s, from about 5 to about 18 mPa-s, from about 5 to about 17 mPa-s, from about 5 to about 16 mPa-s, from about 5 to about 15 mPa-s, from about 5 to about 14 mPa-s, from about 5 to about 13 mPa-s, from about 5 to about 12 mPa-s, from about 5 to about 11 mPa-s, from about 5 to about 10 mPa-s, from about 5 to about 9 mPa-s, from about 5 to about 8 mPa-s, from about 5 to about 7 mPa-s, from about 5 to about 6 mPa-s, from about 6 to about 20 mPa-s, from about 6 to about 19 mPa-s, from about 6 to about 18 mPa-s, from about 6 to about 17 mPa-s, from about 6 to about 16 mPa-s, from about 6 to about 15 mPa-s, from about 6 to about 14 mPa-s, from about 6 to about 13 mPa-s, from about 6 to about 12 mPa-s, from about 6 to about 11 mPa-s, from about 6 to about 10 mPa-s, from about 6 to about 9 mPa-s, from about 6 to about 8 mPa-s, or from about 6 to about 7 mPa-s. In some embodiments, greater than about 100, 125, 150, 160, 170, 180, 190, or 200 mg/ml is up to about 250 mg/ml.

Additionally, for example, in some embodiments, the anti-TL1A antibody is characterized by a turbidity less than 12 Nephelometric Turbidity Units (NTU) when at a concentration greater than about 100 mg/mL e.g., about 150 mg/mL to about 300 mg/mL, about 150 mg/mL to about 200 mg/mL, about 150 mg/mL to about 225 mg/mL, or about 150 mg/mL to about 250 mg/mL. In some cases, the antibody comprises a HCDR1 comprising SEQ ID NO: 1, a HCDR2 comprising SEQ ID NO: 2, a HCDR3 comprising SEQ ID NO: 6, a LCDR1 comprising SEQ ID NO: 10, a LCDR2 comprising SEQ ID NO: 11, and a LCDR3 comprising SEQ ID NO: 12, and/or having a heavy chain variable region comprising SEQ ID NO: 104 and a light chain variable region comprising SEQ ID NO: 201. In some cases, the anti-TL1A antibody comprises a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework. In some embodiments, the anti-TL1A antibody is characterized by a turbidity less than 12 Nephelometric Turbidity Units (NTU) when at a concentration greater than at least about 150 mg/mL. In some embodiments, the anti-TL1A antibody is characterized by a turbidity less than 12 Nephelometric Turbidity Units (NTU) when at a concentration greater than at least about 160 mg/mL. In some embodiments, the anti-TL1A antibody is characterized by a turbidity less than 12 Nephelometric Turbidity Units (NTU) when at a concentration greater than at least about 170 mg/mL. In some embodiments, the anti-TL1A antibody is characterized by a turbidity less than 12 Nephelometric Turbidity Units (NTU) when at a concentration greater than at least about 180 mg/mL. In some embodiments, the anti-TL1A antibody is characterized by a turbidity less than 12 Nephelometric Turbidity Units (NTU) when at a concentration greater than at least about 190 mg/mL. In some embodiments, the anti-TL1A antibody is characterized by a turbidity less than 12 Nephelometric Turbidity Units (NTU) when at a concentration of about 150 mg/mL to about 250 mg/mL. Less than 12 NTU may include about 1, 2, 3, 4, or 5 NTU to about 12 NTU.

Additionally, anti-TL1A antibodies described herein also demonstrate advantageous aggregation properties. In some embodiments, the anti-TL1A antibody composition is characterized by percent high molecular weight species (e.g., a species having a molecular weight greater than the molecular weight of the monomer) is less than 10% of the composition when the antibody is present in the composition at a concentration greater than about 100 mg/mL, e.g., about 150 mg/mL to about 300 mg/mL, about 150 mg/mL to about 200 mg/mL, about 150 mg/mL to about 225 mg/mL, or about 150 mg/mL to about 250 mg/mL. In some cases, the antibody comprises a HCDR1 comprising SEQ ID NO: 1, a HCDR2 comprising SEQ ID NO: 2, a HCDR3 comprising SEQ ID NO: 6, a LCDR1 comprising SEQ ID NO: 10, a LCDR2 comprising SEQ ID NO: 11, and a LCDR3 comprising SEQ ID NO: 12, and/or having a heavy chain variable region comprising SEQ ID NO: 104 and a light chain variable region comprising SEQ ID NO: 201. In some cases, the anti-TL1A antibody comprises a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise less than 9 amino acid modifications from the human IGHV1-46*02 framework and the human IGKV3-20 framework. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 150 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 160 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 170 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 180 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 190 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 200 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 210 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 220 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 230 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 240 mg/mL. In some embodiments, the anti-TL1A antibody composition is characterized by percent high molecular weight species less than 10%, 9%, 8%, 7%, 6%, 5%, 4%, 3%, 2%, or 1% when at a concentration greater than at least about 250 mg/mL. In some embodiments, the anti-TL1A antibody composition is characterized by percent high molecular weight species less than 10%, 9%, 8%, 7%, 6%, 5%, 4%, 3%, 2%, or 1% when at a concentration of about 150 mg/mL to about 250 mg/mL.

In some embodiments, provided are pharmaceutical compositions comprising about 150 mg to about 225 mg of anti-TL1A or anti-IL23 in a total volume of less than or equal to about 1 mL. The composition may be formulated for subcutaneous administration. In some cases, the composition comprises about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of anti-TL1A or anti-IL23. The total volume may be less than about 1.0, 0.9, or 0.8 mL if less than 300 mg of anti-TL1A or anti-IL23. The total volume may be at least about 0.5 mL if less than 300 mg of anti-TL1A or anti-IL23. The total volume may be about 0.5 mL to about 3 mL, about 0.5 mL to about 2.9 mL, about 0.5 mL to about 2.8 mL, about 0.5 mL to about 2.7 mL, about 0.5 mL to about 2.6 mL, about 0.5 mL to about 2.5 mL, about 0.5 mL to about 2.4 mL, about 0.5 mL to about 2.3 mL, about 0.5 mL to about 2.2 mL, about 0.5 mL to about 2.1 mL, about 0.5 mL to about 2 mL, 0.5 mL to about 1.9 mL, 0.5 mL to about 1.8 mL, 0.5 mL to about 1.7 mL, 0.5 mL to about 1.6 mL, about 0.5 mL to about 1.0 mL, about 0.5 mL to about 0.9 mL, about 0.5 mL to about 0.8 mL, about 0.6 mL to about 3 mL, about 0.6 mL to about 2.9 mL, about 0.6 mL to about 2.8 mL, about 0.6 mL to about 2.7 mL, about 0.6 mL to about 2.6 mL, about 0.6 mL to about 2.5 mL, about 0.6 mL to about 2.4 mL, about 0.6 mL to about 2.3 mL, about 0.6 mL to about 2.2 mL, about 0.6 mL to about 2.1 mL, about 0.6 mL to about 2.0 mL, about 0.6 mL to about 1.9 mL, about 0.6 mL to about 1.8 mL, about 0.6 mL to about 1.7 mL, about 0.6 mL to about 1.6 mL, about 0.6 mL to about 1.5 mL, about 0.6 mL to about 1.4 mL, about 0.6 mL to about 1.3 mL, about 0.6 mL to about 1.2 mL, about 0.6 mL to about 1.1 mL, about 0.6 mL to about 1.0 mL, about 0.6 mL to about 0.9 mL, about 0.6 mL to about 0.8 mL, about 0.7 mL to about 3 mL, about 0.7 mL to about 2.9 mL, about 0.7 mL to about 2.8 mL, about 0.7 mL to about 2.7 mL, about 0.7 mL to about 2.6 mL, about 0.7 mL to about 2.5 mL, about 0.7 mL to about 2.4 mL, about 0.7 mL to about 2.3 mL, about 0.7 mL to about 2.2 mL, about 0.7 mL to about 2.1 mL, about 0.7 mL to about 2.0 mL, about 0.7 mL to about 1.9 mL, about 0.7 mL to about 1.8 mL, about 0.7 mL to about 1.7 mL, about 0.7 mL to about 1.6 mL, about 0.7 mL to about 1.5 mL, about 0.7 mL to about 1.4 mL, about 0.7 mL to about 1.3 mL, about 0.7 mL to about 1.2 mL, about 0.7 mL to about 1.1 mL, about 0.7 mL to about 1.0 mL, about 0.7 mL to about 0.9 mL, about 0.7 mL to about 0.8 mL, about 3 mL to about 10 mL, about 3 mL to about 9.5 mL, about 3 mL to about 9.0 mL, about 3 mL to about 8.5 mL, about 3 mL to about 8.0 mL, about 3 mL to about 7.5 mL, about 3 mL to about 7.0 mL, about 3 mL to about 6.5 mL, about 3 mL to about 6 mL, about 3 mL to about 5.5 mL, about 3 mL to about 5.0 mL, about 3 mL to about 4.5 mL, about 3 mL to about 4 mL, about 3 mL to about 3.5 mL, about 3.5 mL to about 10 mL, about 3.5 mL to about 9.5 mL, about 3.5 mL to about 9.0 mL, about 3.5 mL to about 8.5 mL, about 3.5 mL to about 8.0 mL, about 3.5 mL to about 7.5 mL, about 3.5 mL to about 7.0 mL, about 3.5 mL to about 6.5 mL, about 3.5 mL to about 6 mL, about 3.5 mL to about 5.5 mL, about 3.5 mL to about 5.0 mL, about 3.5 mL to about 4.5 mL, about 3.5 mL to about 4 mL, about 4.0 mL to about 10 mL, about 4.0 mL to about 9.5 mL, about 4.0 mL to about 9.0 mL, about 4.0 mL to about 8.5 mL, about 4.0 mL to about 8.0 mL, about 4.0 mL to about 7.5 mL, about 4.0 mL to about 7.0 mL, about 4.0 mL to about 6.5 mL, about 4.0 mL to about 6 mL, about 4.0 mL to about 5.5 mL, about 4.0 mL to about 5.0 mL, about 4.0 mL to about 4.5 mL, about 4.5 mL to about 10 mL, about 4.5 mL to about 9.5 mL, about 4.5 mL to about 9.0 mL, about 4.5 mL to about 8.5 mL, about 4.5 mL to about 8.0 mL, about 4.5 mL to about 7.5 mL, about 4.5 mL to about 7.0 mL, about 4.5 mL to about 6.5 mL, about 4.5 mL to about 6 mL, about 4.5 mL to about 5.5 mL, about 4.5 mL to about 5.0 mL, about 5 mL to about 10 mL, about 5 mL to about 9.5 mL, about 5 mL to about 9.0 mL, about 5 mL to about 8.5 mL, about 5 mL to about 8.0 mL, about 5 mL to about 7.5 mL, about 5 mL to about 7.0 mL, about 5 mL to about 6.5 mL, about 5 mL to about 6 mL, about 5 mL to about 5.5 mL, about 5.5 mL to about 10 mL, about 5.5 mL to about 9.5 mL, about 5.5 mL to about 9.0 mL, about 5.5 mL to about 8.5 mL, about 5.5 mL to about 8.0 mL, about 5.5 mL to about 7.5 mL, about 5.5 mL to about 7.0 mL, about 5.5 mL to about 6.5 mL, about 5.5 mL to about 6 mL, about 6.0 mL to about 10 mL, about 6.0 mL to about 9.5 mL, about 6.0 mL to about 9.0 mL, about 6.0 mL to about 8.5 mL, about 6.0 mL to about 8.0 mL, about 6.0 mL to about 7.5 mL, about 6.0 mL to about 7.0 mL, about 6.0 mL to about 6.5 mL, about 6.5 mL to about 10 mL, about 6.5 mL to about 9.5 mL, about 6.5 mL to about 9.0 mL, about 6.5 mL to about 8.5 mL, about 6.5 mL to about 8.0 mL, about 6.5 mL to about 7.5 mL, about 6.5 mL to about 7.0 mL, about 7.0 mL to about 10 mL, about 7.0 mL to about 9.5 mL, about 7.0 mL to about 9.0 mL, about 7.0 mL to about 8.5 mL, about 7.0 mL to about 8.0 mL, about 7.0 mL to about 7.5 mL, about 7.5 mL to about 10 mL, about 7.5 mL to about 9.5 mL, about 7.5 mL to about 9.0 mL, about 7.5 mL to about 8.5 mL, about 7.5 mL to about 8.0 mL, about 8.0 mL to about 10 mL, about 8.0 mL to about 9.5 mL, about 8.0 mL to about 9.0 mL, about 8.0 mL to about 8.5 mL, about 8.5 mL to about 10 mL, about 8.5 mL to about 9.5 mL, about 8.5 mL to about 9.0 mL, about 9 mL to about 10 mL, about 9 mL to about 9.5 mL, or about 9.5 mL to about 10 mL. In some embodiments, the concentration of anti-TL1A or anti-IL23 is about or greater than about 55, 60, 65, 70, 75, 80, 85, 90, 95, 100, 105, 110, 115, 120, 125, 130, 135, 140, 145, 155, 160, 165, 170, 175, 180, 185, 190, 195, 200, 205, 210, 215, 220, 225, 230, 235, 240, 245, or 250 mg/mL.

In some embodiments, provided are pharmaceutical compositions comprising about 400 mg to about 1000 mg or 400 mg to 2000 mg of anti-TL1A or anti-IL23 in a total volume of less than or equal to about 15 mL. The composition may be formulated for intravenous administration. The composition may be diluted into about 100 to about 300, or about 250 mL pharmaceutically acceptable solution (e.g., saline) for intravenous administration. The total volume may be at least about 1 mL, at least about 2 mL, at least about 2.5 mL, at least about 3 mL, at least about 4 mL, or at least about 5 mL; and less than or equal to about 15 mL, 14 mL, 13 mL, 11 mL, or 10 mL. For instance, the volume may be from about 1 mL to about 15 mL, from about 1 mL to about 14 mL, from about 1 mL to about 13 mL, from about 1 mL to about 12 mL, from about 1 mL to about 11 mL, from about 1 mL to about 10 mL, from about 1 mL to about 9 mL, from about 1 mL to about 8 mL, from about 1 mL to about 7 mL, from about 1 mL to about 6 mL, from about 1 mL to about 5 mL, from about 1 mL to about 4 mL, from about 1 mL to about 3 mL, from about 1 mL to about 2 mL, from about 2 mL to about 15 mL, from about 2 mL to about 14 mL, from about 2 mL to about 13 mL, from about 2 mL to about 12 mL, from about 2 mL to about 11 mL, from about 2 mL to about 10 mL, from about 2 mL to about 9 mL, from about 2 mL to about 8 mL, from about 2 mL to about 7 mL, from about 2 mL to about 6 mL, from about 2 mL to about 5 mL, from about 2 mL to about 4 mL, from about 2 mL to about 3 mL, from about 3 mL to about 15 mL, from about 3 mL to about 14 mL, from about 3 mL to about 13 mL, from about 3 mL to about 12 mL, from about 3 mL to about 11 mL, from about 3 mL to about 10 mL, from about 3 mL to about 9 mL, from about 3 mL to about 8 mL, from about 3 mL to about 7 mL, from about 3 mL to about 6 mL, from about 3 mL to about 5 mL, from about 3 mL to about 4 mL, from about 4 mL to about 15 mL, from about 4 mL to about 14 mL, from about 4 mL to about 13 mL, from about 4 mL to about 12 mL, from about 4 mL to about 11 mL, from about 4 mL to about 10 mL, from about 4 mL to about 9 mL, from about 4 mL to about 8 mL, from about 4 mL to about 7 mL, from about 4 mL to about 6 mL, from about 4 mL to about 5 mL, from about 5 mL to about 15 mL, from about 5 mL to about 14 mL, from about 5 mL to about 13 mL, from about 5 mL to about 12 mL, from about 5 mL to about 11 mL, from about 5 mL to about 10 mL, from about 5 mL to about 9 mL, from about 5 mL to about 8 mL, from about 5 mL to about 7 mL, or from about 5 mL to about 6 mL.

### Non-Limiting Example Excipients

In certain embodiments, a pharmaceutical composition comprising a TL1A inhibitor (e.g. an anti-TL1A antibody or antigen-binding fragment thereof) or an IL23 inhibitor (e.g. an anti-IL23 antibody or antigen-binding fragment thereof) comprises a surfactant. A surfactant includes a nonionic surfactant, ionic surfactant, and amphoteric surfactant, and combinations thereof. In some embodiments, the surfactant comprises a nonionic surfactant. Non-limiting examples of non-ionic surfactants include polysorbate, polyglycerol alkyl ether, glucosyl dialkyl ether, crownether, ester-linked surfactant, polyoxyethylene alkyl ether, poloxamer 18, Brij, Spans (sorbitan ester), Triton X-100 (polyethylene glycol p-(1,1,3,3-tetramethylbutyl)-phenyl ether), polyoxyethylene (35) dodecyl ether, polyethylene glycol hexadecyl ether, polyoxyethylene (20) oleyl ether, polyoxyethylene (9) lauryl alcohol, polyethoxylated (35) castor oil, octylphenoxypoly(ethyleneoxy) ethanol, poly(oxyethylene-cooxypropylene) block copolymer, poly(oxyethylene-cooxypropylene) block copolymer, poly(oxyethylene-cooxypropylene) block copolymer, polydimethylsiloxane methylethoxylate, p-Isononylphenoxy-poly(glycidol), 2,4,7,9-tetramethyl-5-decyne-4,7-diol ethoxylate, polyethylene glycol-polypropylene glycol-polyethyleneglycol triblock polymer, and nonylphenol ethoxylate, and combinations thereof. In some embodiments, the surfactant comprises an ionic surfactant. Ionic surfactants include anionic and cationic surfactants. Non-limiting examples of anionic surfactants include alkyl sulfate, alkyl ether sulfate, docusate, sulfonate fluorosurfactant, alkyl benzene sulfonate, alkyl aryl ether phosphate, alkyl ether phosphate, alkyl carboxylate, and sodium dioctyl-sulfosuccinate, and combinations thereof. Non-limiting examples of cationic surfactants include cetyltrimethylammonium bromide (CTAB), cetyltrimethylammonium chloride (CTAC), cetylpyridinium chloride (CPC), polyethoxylated tallow amine (POEA), benzalkonium chloride (BAC), benzethonium chloride (BZT), 5-bromo-5-nitro-1,3-dioxane, dimethyl dioctadecyl ammonium chloride, and dioctadecyl dimethyl ammonium bromide (DODAB), and combinations thereof. In some embodiments, the surfactant comprises an amphoteric surfactant. An example amphoteric surfactant includes ethylenediamine tetrakis (ethoxylate-block-propoxylate) tetrol.

In example embodiments, the surfactant comprises polysorbate. Polysorbate includes, without limitation, polysorbate-20, polysorbate-60, and polysorbate-80, and combinations thereof. The polysorbate may be polysorbate-20.

In some embodiments of the composition provided herein, the composition comprises a surfactant, wherein the surfactant comprises or consists of polysorbate-20. In some embodiments of the composition provided herein, the surfactant comprises or consists of polysorbate-20.

In some embodiments, the surfactant is present in the composition at a concentration of about 0.001-0.1% v/v of the composition. For instance, the surfactant is present at a concentration of about 0.005% to about 0.05%, about 0.01% to about 0.05%, about 0.005% to about 0.04%, about 0.01% to about 0.04%, about 0.005% to about 0.03%, about 0.01% to about 0.03%, about 0.005% to about 0.02%, or about 0.01% to about 0.02% v/v of the composition. In example embodiments, the surfactant comprises about 0.01% to about 0.05%, or about 0.01%, about 0.02%, about 0.03%, about 0.04%, or about 0.05% v/v of the composition. As a further embodiment, the surfactant comprises about 0.01% to about 0.05%, or about 0.01%, about 0.02%, about 0.03%, about 0.04%, or about 0.05% polysorbate in the composition. For instance, some embodiments of the compositions comprise about 0.01%-0.02%, or about 0.01% or about 0.02% polysorbate. In one embodiment of the composition provided herein, the composition comprises polysorbate-20 at a concentration of about 0.01% to about 0.05%, or about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, about 0.02%, about 0.021%, about 0.022%, about 0.023%, about 0.024%, about 0.025%, about 0.026%, about 0.027%, about 0.028%, about 0.029%, or about 0.03% v/v of the composition. In one embodiment of the composition provided herein, the composition comprises polysorbate-20 at a concentration of about 0.02% v/v of the composition. In one embodiment of the composition provided herein, the composition comprises polysorbate-60 at a concentration of about 0.01% to about 0.05%, or about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, about 0.02%, about 0.021%, about 0.022%, about 0.023%, about 0.024%, about 0.025%, about 0.026%, about 0.027%, about 0.028%, about 0.029%, or about 0.03% v/v of the composition. In one embodiment of the composition provided herein, the composition comprises polysorbate-60 at a concentration of about 0.02% v/v of the composition. In one embodiment of the composition provided herein, the composition comprises polysorbate-80 at a concentration of about 0.01% to about 0.05%, or about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, about 0.02%, about 0.021%, about 0.022%, about 0.023%, about 0.024%, about 0.025%, about 0.026%, about 0.027%, about 0.028%, about 0.029%, or about 0.03% v/v of the composition. In one embodiment of the composition provided herein, the composition comprises polysorbate-80 at a concentration of about 0.02% v/v of the composition.

In certain embodiments, a pharmaceutical composition comprising a TL1A inhibitor (e.g. an anti-TL1A antibody or antigen-binding fragment thereof) or an IL23 inhibitor (e.g. an anti-IL23 antibody or antigen-binding fragment thereof) comprises a stabilizer. Stabilizers include sugars, polyols, amino acids, polymers, and cyclodextrin (e.g., HP-b-CD), and combinations thereof. In some embodiments, the stabilizer comprises a sugar. Non-limiting examples of sugars include sucrose, glucose, trehalose, maltose, and lactose, and combinations thereof. In some embodiments, the stabilizer comprises a polyol. Non-limiting examples of polyols include mannitol, sorbitol, raffinose, and glycerol, and combinations thereof. In exemplary embodiments, the stabilizer comprises a sugar, such as sucrose. In some embodiments, the sugar comprises or consists of sucrose. In some embodiments, the stabilizer comprises an amino acid. In some embodiments, the amino acid comprises or consists of glycine. In some embodiments, the amino acid comprises or consists of glycine. In some embodiments, the stabilizer comprises both a sugar and an amino acid. In some embodiments, the stabilizer comprises both sucrose and glycine.

In some embodiments, the stabilizer is present in the composition at a concentration of about 50 mM to about 300 mM. For instance, the stabilizer is present at a concentration of about 50 mM to about 300 mM, about 50 mM to about 290 mM, about 50 mM to about 280 mM, about 50 mM to about 270 mM, about 50 mM to about 260 mM, about 50 mM to about 250 mM, about 50 mM to about 240 mM, about 50 mM to about 220 mM, about 50 mM to about 200 mM, about 75 mM to about 300 mM, about 75 mM to about 290 mM, about 75 mM to about 280 mM, about 75 mM to about 270 mM, about 75 mM to about 260 mM, about 75 mM to about 250 mM, about 75 mM to about 240 mM, about 75 mM to about 220 mM, about 75 mM to about 200 mM, about 100 mM to about 300 mM, about 100 mM to about 290 mM, about 100 mM to about 280 mM, about 100 mM to about 270 mM, about 100 mM to about 260 mM, about 100 mM to about 250 mM, about 100 mM to about 240 mM, about 100 mM to about 220 mM, about 100 mM to about 200 mM, about 125 mM to about 300 mM, about 125 mM to about 290 mM, about 125 mM to about 280 mM, about 125 mM to about 270 mM, about 125 mM to about 260 mM, about 125 mM to about 250 mM, about 125 mM to about 240 mM, about 125 mM to about 220 mM, about 125 mM to about 200 mM, about 150 mM to about 300 mM, about 150 mM to about 290 mM, about 150 mM to about 280 mM, about 150 mM to about 270 mM, about 150 mM to about 260 mM, about 150 mM to about 250 mM, about 150 mM to about 240 mM, about 150 mM to about 220 mM, about 150 mM to about 200 mM, about 175 mM to about 300 mM, about 175 mM to about 290 mM, about 175 mM to about 280 mM, about 175 mM to about 270 mM, about 175 mM to about 260 mM, about 175 mM to about 250 mM, about 175 mM to about 240 mM, about 175 mM to about 220 mM, about 175 mM to about 200 mM, about 200 mM to about 300 mM, about 200 mM to about 290 mM, about 200 mM to about 280 mM, about 200 mM to about 270 mM, about 200 mM to about 260 mM, about 200 mM to about 250 mM, about 200 mM to about 240 mM, or about 200 mM to about 220 mM. In example embodiments, the stabilizer is present at concentrations of about 150 mM to about 270 mM, or about 60, 65, 70, 75, 80, 85, 90, 95, 100, 105, 110, 115, 120, 125, 130, 135, 140, 145, 150, 160, 170, 180, 190, 200, 210, 220, 230, 240, 250, 260, or 270 mM stabilizer. As a further embodiment, the composition comprises about 150 mM to about 270 mM, or about 150, 160, 170, 180, 190, 200, 210, 220, 230, 240, 250, 260, or 270 mM sucrose, for instance, about 220-240 mM, or about 220, about 230, or about 240 mM sucrose. In yet another embodiment, the composition comprises about 50 mM to about 150 mM, or about 50, 55, 60, 65, 70, 75, 80, 85, 90, 95, 100, 105, 110, 115, 120, 125, 130, 135, 140, 145, 150 mM glycine, for instance, 75-100 mM or about 80, about 85, or about 90 mM glycine. In yet another embodiment, the composition comprises about 150 mM to about 270 mM, or about 150, 160, 170, 180, 190, 200, 210, 220, 230, 240, 250, 260, or 270 mM sucrose and comprises 50 mM to about 150 mM, or about 50, 55, 60, 65, 70, 75, 80, 85, 90, 95, 100, 105, 110, 115, 120, 125, 130, 135, 140, 145, 150 mM glycine.

In certain embodiments, a pharmaceutical composition comprising a TL1A inhibitor (e.g. an anti-TL1A antibody or antigen-binding fragment thereof) or an IL23 inhibitor (e.g. an anti-IL23 antibody or antigen-binding fragment thereof) comprises a salt. Non-limiting examples of salt include sodium chloride, glycine, lysine-hydrochloride, arginine-hydrochloride, arginine glutamate, potassium chloride, magnesium chloride, and calcium chloride, and combinations thereof. In some embodiments, the salt comprises sodium chloride. In some embodiments, the salt comprises lysine-HCl.

In some embodiments, the salt is present in the composition at a concentration of about 10 mM to about 150 mM. For instance, the salt is present at a concentration of about 10 mM to about 150 mM, about 10 mM to about 140 mM, about 10 mM to about 130 mM, about 10 mM to about 120 mM, about 10 mM to about 110 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 20 mM to about 150 mM, about 20 mM to about 140 mM, about 20 mM to about 130 mM, about 20 mM to about 120 mM, about 20 mM to about 110 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 150 mM, about 30 mM to about 140 mM, about 30 mM to about 130 mM, about 30 mM to about 120 mM, about 30 mM to about 110 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 150 mM, about 40 mM to about 140 mM, about 40 mM to about 130 mM, about 40 mM to about 120 mM, about 40 mM to about 110 mM, about 40 mM to about 100 mM, about 40 mM to about 90 mM, about 40 mM to about 80 mM, about 40 mM to about 70 mM, about 40 mM to about 60 mM, or about 40 mM to about 50 mM. In example embodiments, the salt is present at concentrations of about 25 mM to about 130 mM. As a further embodiment, the composition comprises about 40 mM to about 130 mM NaCl. For instance, the composition comprises about 40 mM NaCl. In some embodiments, the composition comprises about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, or about 150 mM NaCl. As a further embodiment, the composition comprises about 25 mM to about 50 mM Lys-HCl. For instance, the composition comprises about 25 mM Lys-HCl.

In certain embodiments, a pharmaceutical composition comprising a TL1A inhibitor (e.g. an anti-TL1A antibody or antigen-binding fragment thereof) or an IL23 (e.g. an anti-IL23 antibody or antigen-binding fragment thereof) inhibitor comprises a buffering agent. Non-limiting examples of buffering agents include an acetate, phosphate, citrate, glutamate, succinate, gluconate, histidine, glycylglycine, citric acid, Tris (tris (hydroxymethyl) aminomethane), and diethanolamine, and combinations thereof. In an example embodiment, the buffering agent comprises acetate. In some embodiments, the buffering agent comprises sodium acetate. In some embodiments, the buffering agent comprises acetic acid. In some embodiments, the buffering agent comprising acetate comprises acetic acid and sodium acetate. In some embodiments, the buffering agent comprises potassium acetate. In some embodiments, the buffering agent comprises aluminum acetate. In some embodiments, the buffering agent comprises ammonium acetate. In some embodiments, the buffering agent comprises phosphate. In one embodiment, the buffering agent comprising phosphate comprises phosphoric acid and sodium phosphate. In some embodiments, the buffering agent comprises phosphoric acid and potassium phosphate. In some embodiments, the buffering agent comprises sodium phosphate dibasic and sodium phosphate monobasic. In some embodiments, the buffering agent comprises phosphoric acid, sodium phosphate dibasic, sodium phosphate monobasic, and/or sodium phosphate. In some embodiments, the buffering agent comprises potassium phosphate dibasic and potassium phosphate monobasic. In some embodiments, the buffering agent comprises phosphoric acid, potassium phosphate dibasic, potassium phosphate monobasic, and/or potassium phosphate. In some embodiments, the buffering agent is present in the composition at a concentration of about 5 mM to about 50 mM. For instance, the buffering agent is present at a concentration of about 5 mM to about 50 mM, about 5 mM to about 40 mM, about 5 mM to about 30 mM, about 5 mM to about 20 mM, about 5 mM to about 10 mM, about 10 mM to about 50 mM, about 10 mM to about 40 mM, about 10 mM to about 30 mM, or about 10 mM to about 20 mM. As a non-limiting example, the buffering agent is present at a concentration of about 10 mM to about 20 mM, or about 20 mM. As a further example embodiment, the composition comprises about 10 mM to about 20 mM, or about 10 mM or about 20 mM of acetate. In a further embodiment, the composition comprises about 10 mM to about 20 mM, or about 10 mM or about 20 mM of phosphate.

In certain embodiments, a pharmaceutical composition comprising a TL1A inhibitor (e.g. an anti-TL1A antibody or antigen-binding fragment thereof) or an IL23 inhibitor (e.g. an anti-IL23 antibody or antigen-binding fragment thereof) has a pH of 4.0 to 8.0. For instance, the pH is about 4.5 to about 8.0, about 4.5 to about 7.8, about 4.5 to about 7.6, about 4.5 to about 7.4, about 4.5 to about 7.2, about 4.5 to about 7.0, about 4.5 to about 6.8, about 4.5 to about 6.6, about 4.5 to about 6.4, about 4.5 to about 6.2, about 4.5 to about 6.0, about 4.5 to about 5.8, about 4.5 to about 5.6, about 4.5 to about 5.4, about 4.5 to about 5.2, or about 4.5 to about 5.0. In some embodiments, the pH is about 4.5 to about 6.0, about 4.5 to about 5.9, about 4.5 to about 5.8, about 4.5 to about 5.7, or about 4.5 to about 5.6. In example embodiments, the pH is about 4.5 to about 5.5, or about 4.5 to about 5.4, about 4.5 to about 5.3, about 4.5 to about 5.2, about 4.5 to about 5.1, about 4.5 to about 5.0, 4.6 to about 5.5, about 4.6 to about 5.4, about 4.6 to about 5.3, about 4.6 to about 5.2, about 4.6 to about 5.1, about 4.6 to about 5.0, 4.7 to about 5.5, about 4.7 to about 5.4, about 4.7 to about 5.3, about 4.7 to about 5.2, about 4.7 to about 5.1, about 4.7 to about 5.0, 4.8 to about 5.5, about 4.8 to about 5.4, about 4.8 to about 5.3, about 4.8 to about 5.2, about 4.8 to about 5.1, about 4.8 to about 5.0, 4.9 to about 5.5, about 4.9 to about 5.4, about 4.9 to about 5.3, about 4.9 to about 5.2, about 4.9 to about 5.1, about 4.9 to about 5.0, about 5.0 to about 5.5, about 5.0 to about 5.4, about 5.0 to about 5.3, about 5.0 to about 5.2, about 5.0 to about 5.1, about 5.1 to about 5.5, about 5.1 to about 5.4, about 5.1 to about 5.3, about 5.1 to about 5.2, about 5.2 to about 5.5, about 5.2 to about 5.4, about 5.2 to about 5.3, about 5.3 to about 5.5, about 5.3 to about 5.4, or about 5.4 to about 5.5. The pH may be about 4.5 to about 5.5, or about 4.5, 4.6, 4.7, 4.8, 4.9, 5.0, 5.1, 5.2, 5.3, 5.4, or 5.5. As an example, the pH is about 5.3. In a non-limiting example, the composition comprises an acetate buffer, with a pH of about 4.5 to about 5.5, or about 5.3. In certain embodiments, the pH is about 6.0 to about 7.0, about 6.0 to about 6.9, about 6.0 to about 6.8, about 6.0 to about 6.7, about 6.0 to about 6.6, about 6.0 to about 6.5, about 6.0 to about 6.4, about 6.0 to about 6.3, about 6.0 to about 6.2, about 6.0 to about 6.1, about 6.1 to about 7.0, about 6.1 to about 6.9, about 6.1 to about 6.8, about 6.1 to about 6.7, about 6.1 to about 6.6, about 6.1 to about 6.5, about 6.1 to about 6.4, about 6.1 to about 6.3, about 6.1 to about 6.2, about 6.2 to about 7.0, about 6.2 to about 6.9, about 6.2 to about 6.8, about 6.2 to about 6.7, about 6.2 to about 6.6, about 6.2 to about 6.5, about 6.2 to about 6.4, about 6.2 to about 6.3, about 6.3 to about 7.0, about 6.3 to about 6.9, about 6.3 to about 6.8, about 6.3 to about 6.7, about 6.3 to about 6.6, about 6.3 to about 6.5, about 6.3 to about 6.4, about 6.4 to about 7.0, about 6.4 to about 6.9, about 6.4 to about 6.8, about 6.4 to about 6.7, about 6.4 to about 6.6, about 6.4 to about 6.5, about 6.5 to about 7.0, about 6.5 to about 6.9, about 6.5 to about 6.8, about 6.5 to about 6.7, or about 6.5 to about 6.6. The pH can be about 6.0 to about 7.0, or about 6.1, 6.2, 6.3, 6.4, 6.5, 6.6, 6.7, 6.8, 6.9, or 7.0. As an example, the pH is about 6.5. In a non-limiting example, the composition comprises a phosphate buffer, with a pH of about 6.0 to about 7.0, or about 6.5.

In some embodiments, a pharmaceutical composition comprising a TL1A inhibitor (e.g. an anti-TL1A antibody or antigen-binding fragment thereof) or an IL23 inhibitor (e.g. an anti-IL23 antibody or antigen-binding fragment thereof) comprises one or more of the following: surfactant, stabilizer, salt, and buffering agent. In some embodiments, the pharmaceutical composition comprises a surfactant and a stabilizer. In some embodiments, the pharmaceutical composition comprises a surfactant and a salt. In some embodiments, the pharmaceutical composition comprises a surfactant and a buffering agent. In some embodiments, the pharmaceutical composition comprises a stabilizer and a salt. In some embodiments, the pharmaceutical composition comprises a stabilizer and a buffering agent. In some embodiments, the pharmaceutical composition comprises a salt and buffering agent. In some embodiments, the pharmaceutical composition comprises a surfactant, stabilizer, and salt. In some embodiments, the pharmaceutical composition comprises surfactant, salt, and buffering agent. In some embodiments, the pharmaceutical composition comprises a surfactant, stabilizer and buffering agent. In some embodiments, the pharmaceutical composition comprises a stabilizer, salt, and buffering agent. In some embodiments, the pharmaceutical composition comprises a surfactant, stabilizer, salt, and buffering agent.

Non-limiting example pharmaceutical compositions comprise a nonionic surfactant, sugar, salt and buffering agent. For instance, the compositions comprise polysorbate (e.g., polysorbate-20), sucrose, lysine-HCl or sodium chloride, and an acetate buffer. The pH of the composition may be about 4.5 to about 5.5, or about 5.0 to about 5.5. In an example embodiment, the composition comprises about 10⁻²⁰ mM acetate at pH 4.5-5.5, 150-270 mM sucrose, 25-50 mM Lys-HCl, and 0.01%-0.05% v/v polysorbate-20. For instance, the composition comprises about 20 mM acetate at pH 5.3, about 240 mM sucrose, about 25 mM lysine-HCl, and about 0.02% polysorbate-20. As another example embodiment, the composition comprises about 10⁻²⁰ mM acetate at pH 4.5-5.5, 150-270 mM sucrose, 50-130 mM NaCl, and 0.01%-0.05% v/v polysorbate-20. For instance, the composition comprises about 20 mM acetate at pH 5.3, 220 mM sucrose, 40 mM NaCl, and 0.02% polysorbate-20.

In some embodiments, the compositions comprise polysorbate (e.g., polysorbate-20), sucrose, sodium chloride, and an acetate buffer. The pH of the composition may be about 4.5 to about 5.5, or about 5.0 to about 5.5. In an example embodiment, the composition comprises about 10⁻²⁰ mM acetate at pH 4.5-5.5, 150-270 mM sucrose, and 0.01%-0.05% v/v polysorbate-20. For instance, the composition comprises about 20 mM acetate at pH 5.3, about 220 mM sucrose, and about 0.02% polysorbate-20. As another example embodiment, the composition comprises about 10⁻²⁰ mM acetate at pH 4.5-5.5, 150-270 mM sucrose, 50-130 mM NaCl, and 0.01%-0.05% v/v polysorbate-20. For instance, the composition comprises about 20 mM acetate at pH 5.3, 220 mM sucrose, 40 mM NaCl, and 0.02% polysorbate-20.

In some embodiments, the compositions comprise polysorbate (e.g., polysorbate-20), sucrose, glycine, sodium chloride, and a phosphate buffer. In certain embodiments, the compositions comprise polysorbate (e.g., polysorbate-20), sucrose, glycine, and a phosphate buffer. In some embodiments, the compositions comprise polysorbate-20, sucrose, glycine, and a phosphate buffer. The pH of the composition may be about 6.0 to about 7.0, or about 6.5 to about 7.0. In an example embodiment, the composition comprises about 10⁻²⁰ mM phosphate at pH 6.0-7.0, 75-100 mM glycine, 100-270 mM sucrose, and 0.01%-0.05% v/v polysorbate-20. For instance, the composition comprises about 20 mM phosphate at pH 6.5, about 85 mM glycine, about 146 mM sucrose, and about 0.02% polysorbate-20. As another example embodiment, the composition comprises about 10⁻²⁰ mM phosphate at pH 6.0-7.0, 75-100 mM glycine, 2% to 8% (w/v) sucrose, and 0.01%-0.05% v/v polysorbate-20. For instance, the composition comprises about 20 mM phosphate at pH 6.5, 5% (w/v) sucrose, 85 mM glycine, and 0.02% polysorbate-20.

In one embodiment, provided herein is a composition comprising an anti-TL1A antibody provided herein at a concentration of about 200 mg/mL, 20 mM sodium acetate, 220 mM sucrose, 40 mM NaCl, and 0.02% polysorbate-20, at pH 5.3. In another embodiment, provided herein is a composition comprising an anti-TL1A antibody provided herein at a concentration of about 100 mg/mL, 20 mM sodium acetate, 220 mM sucrose, 40 mM NaCl, and 0.02% polysorbate-20, at pH 5.3. In another embodiment, provided herein is a composition comprising an anti-TL1A antibody provided herein at a concentration of about 60 mg/mL, 20 mM sodium phosphate, 5% sucrose, 85 mM glycine, and 0.02% polysorbate-20, at pH 5.3. In one embodiment, provided herein is a composition comprising an anti-TL1A antibody provided herein at a concentration described herein, 20 mM sodium acetate, 220 mM sucrose, 40 mM NaCl, and 0.02% polysorbate-20, at pH 5.3. In another embodiment, provided herein is a composition comprising an anti-TL1A antibody provided herein at a concentration described herein, 20 mM sodium acetate, 220 mM sucrose, 40 mM NaCl, and 0.02% polysorbate-20, at pH 5.3. In another embodiment, provided herein is a composition comprising an anti-TL1A antibody provided herein at a concentration described herein, 20 mM sodium phosphate, 5% sucrose, 85 mM glycine, and 0.02% polysorbate-20, at pH 5.3. In one embodiment, provided herein is a composition comprising an anti-TL1A antibody provided herein at a concentration of about 150 mg/ml to 250 mg/ml, 20 mM sodium acetate, 220 mM sucrose, 40 mM NaCl, and 0.02% polysorbate-20, at pH 5.3. In another embodiment, provided herein is a composition comprising an anti-TL1A antibody provided herein at a concentration of about 100 mg/ml to 200 mg/ml, 20 mM sodium acetate, 220 mM sucrose, 40 mM NaCl, and 0.02% polysorbate-20, at pH 5.3. In another embodiment, provided herein is a composition comprising an anti-TL1A antibody provided herein at a concentration of about 50 mg/ml to 100 mg/ml, 20 mM sodium phosphate, 5% sucrose, 85 mM glycine, and 0.02% polysorbate-20, at pH 5.3.

For various embodiments of the composition provided herein, including in this Section (Section 4.5), for example those of the preceding paragraphs), further embodiments of TL1A inhibitors are provided in Section 4.3.1(for example, the anti-TL1A antibodies, including embodiments with exemplary CDRs, framework sequences, constant region sequences, Fc mutations, variable regions, Fc regions, and other properties are further provided in Section 4.3.1(a) and soluble DR3 protein, a variant of soluble DR3 protein, a soluble DR3 protein fused with Fc, or a variant of soluble DR3 protein fused with Fc, each as described in Section 4.3.1(c)); further embodiments for IL23 inhibitors and various doses or dosing regimen for using the IL23 inhibitors are provided in Section 4.3.2; assays for screening, testing, and validating the anti-TL1A or anti-IL23 antibodies are provided in Section 4.3.3; methods for generating, improving, mutating, cloning, expressing, and isolating the anti-TL1A or anti-IL23 antibodies are provided in Section 4.4; methods for using the combination of a TL1A inhibitor and an IL23 inhibitor for treating an inflammatory disease or condition are provided in Sections 2, 4.7, and 5; the therapeutically effective amount (including dose and dosing regimens) for the TL1A inhibitors are provided in Sections 4.6 and 4.7 and this Section (Section 4.5); the therapeutically effective amount (including dose and dosing regimens) for the IL23 inhibitors are provided in Sections 4.3.2 and 4.7 and this Section (Section 4.5); further specific and validated embodiments for the methods of using the combination of TL1A inhibitors and IL23 inhibitors for treating an inflammatory disease or condition are provided in Section 5. As such, the disclosure provides the various combinations of the TL1A inhibitors (including anti-TL1A antibodies and antigen-binding fragments thereof), the IL23 inhibitors, the pharmaceutical compositions of such TL1A inhibitors and/or IL23 inhibitors, the therapeutically effective amounts (such as doses or the dosing regimens for using such pharmaceutical compositions of such TL1A inhibitors and/or IL23 inhibitors, the methods of generating the TL1A inhibitors and/or IL23 inhibitors, the methods of assaying the TL1A inhibitors and/or IL23 inhibitors, and the methods of using the TL1A inhibitors and IL23 inhibitors for the combination therapy.

### 6.6 Additional Dose and Dosing Regimen for TL1A Inhibitor in the Combination Therapy

The disclosure provides that in treating a subject with an inflammatory disease or condition, the mediators of the inflammation are reduced to a level below that in a healthy subject. Accordingly, in one embodiment, effective dose of TL1A inhibitors in the combination therapy reduces the concentration of TL1A in the diseased tissue in the subject with the inflammatory disease or conditions below the concentration of TL1A in a corresponding tissue in a control subject without the inflammatory disease or conditions.

In some embodiments of the effective dose of TL1A inhibitors in the combination therapy provided herein, including in this Section (Section 4.6), the diseased tissue comprises or consists of a tissue in the intestine. In some embodiments of the effective dose of TL1A inhibitors in the combination therapy provided herein, including in this Section (Section 4.6), the diseased tissue comprises or consists of 2, 3, 4, 5, 6, 7, 8, or more tissues in the intestine. In some embodiments of the effective dose of TL1A inhibitors in the combination therapy provided herein, including in this Section (Section 4.6), the corresponding tissue or the reference tissue comprises or consists of a tissue in the intestine. In some embodiments of the effective dose of TL1A inhibitors in the combination therapy provided herein, including in this Section (Section 4.6), the corresponding tissue or the reference tissue comprises or consists of 2, 3, 4, 5, 6, 7, 8, or more tissues in the intestine.

The effective dose of TL1A inhibitors used in combination therapy provided herein, including in this Section (Section 4.6), can be or include various dosing regimens. In some embodiments of effective dose of TL1A inhibitors in the combination therapy provided herein, including in this Section (Section 4.6), the effective dose comprises an induction regimen. In certain embodiments, the effective dose consists of an induction regimen. In some additional embodiments, the effective dose comprises a maintenance regimen. In certain further embodiments, the effective dose comprises an induction regimen and a maintenance regimen. In one embodiment, the effective dose consists of an induction regimen and a maintenance regimen. In some other embodiments, the maintenance regimen is administered in a maintenance step as further described below.

The effective dose for the TL1A inhibitors provided herein, including in this Section (Section 4.6), can include an induction regimen and a maintenance regimen. In some embodiments of the effective dose of TL1A inhibitors in the combination therapy provided herein, including in this Section (Section 4.6), the effective dose further comprises a maintenance regimen that maintains TL1A in the diseased tissue in the subject at a concentration below the concentration of TL1A in the corresponding tissue in the control subject. In certain embodiments, the TL1A in the diseased tissue in the subject is maintained with a maintenance regimen of the TL1A inhibitor. In certain embodiments, the maintenance regimen is administered after the induction regimen.

The disclosure provides that the induction regimen and the maintenance regimen of the TL1A inhibitors in the combination therapy, including in this Section (Section 4.6), can be identical or different in various aspects. In one embodiment of the combination therapy provided herein, including in this Section (Section 4.6), the induction regimen and the maintenance regimen are identical. In another embodiment, the induction regimen and the maintenance regimen are different. In a further embodiment, the induction regimen comprises doses of the TL1A inhibitor higher than the maintenance regimen. In yet another embodiment, the induction regimen comprises doses of the TL1A inhibitor 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, or more fold higher than the maintenance regimen.

As described above and below, the effective dose of the TL1A inhibitors in the combination therapy provided herein can reduce the concentration of TL1A in a diseased tissue in the subject below the concentration of TL1A in a corresponding tissue in a control subject without the inflammatory disease or conditions (such as IBD). Alternatively, the effective dose of TL1A inhibitors in the combination therapy provided herein can reduce the concentration of TL1A in a diseased tissue in the subject below a reference TL1A level (e.g. a reference concentration). Additionally, the effective dose of the TL1A inhibitors in the combination therapy can reduce the concentration of TL1A in a diseased tissue in the subject below the concentration of TL1A in a reference tissue in a control subject without the inflammatory disease or conditions (such as IBD). As is already clear from the description above, the diseased tissue in an IBD patient overproduces TL1A, which contributes to the cause, phenotypes, and/or symptoms of the IBD patient. The effective dose of the TL1A inhibitors in the combination therapy reduces the concentration of TL1A in the diseased tissues of the subject below the concentration of TL1A in a corresponding tissue in a control subject without the inflammatory disease or condition, while the diseased tissues (e.g. certain cells in the diseased tissues) of the subject are overproducing TL1A. Such reduction of TL1A concentration in the diseased tissues of the subject to below (i) a reference TL1A level or (ii) the concentration of TL1A in a corresponding tissue or a reference tissue in a control subject without the inflammatory disease or condition, while the diseased tissue in the subject overproduces TL1A, can also be referred to as coverage. For example, a coverage of or covering 100 fold overproduction of TL1A means that TL1A concentration in the diseased tissues of the subject is reduced to below the concentration of TL1A in a corresponding tissue or a reference tissue in a control subject without the inflammatory disease or condition, while the diseased tissue overproduces TL1A up to 100 fold comparing to the corresponding tissue or the reference tissue in a control subject without the inflammatory disease or condition.

Accordingly, in some embodiments of the effective dose of the TL1A inhibitors in the combination therapy provided herein, including in this Section (Section 4.6), the diseased tissue in the subject produces up to 50, up to 55, up to 60, up to 65, up to 70, up to 75, up to 80, up to 85, up to 90, up to 95, up to 100, up to 105, up to 110, up to 115, up to 120, up to 125, up to 130, up to 135, up to 140, up to 145, up to 150, up to 155, up to 160, up to 165, up to 170, up to 175, up to 180, up to 185, up to 190, up to 195, up to 200 or up to more fold of TL1A compared to the corresponding tissue in the control subject. In certain embodiments, the diseased tissue in the subject produces about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200 or about more fold of TL1A compared to the corresponding tissue in the control subject. In some embodiments, the diseased tissue in the subject produces 20 to 50, 20 to 55, 20 to 60, 20 to 65, 20 to 70, 20 to 75, 20 to 80, 20 to 85, 20 to 90, 20 to 95, 20 to 100, 20 to 105, 20 to 110, 20 to 115, 20 to 120, 20 to 125, 20 to 130, 20 to 135, 20 to 140, 20 to 145, 20 to 150, 20 to 155, 20 to 160, 20 to 165, 20 to 170, 20 to 175, 20 to 180, 20 to 185, 20 to 190, 20 to 195, 20 to 200, or more fold of TL1A compared to the corresponding tissue in the control subject. In some embodiments, the diseased tissue in the subject produces 30 to 50, 30 to 55, 30 to 60, 30 to 65, 30 to 70, 30 to 75, 30 to 80, 30 to 85, 30 to 90, 30 to 95, 30 to 100, 30 to 105, 30 to 110, 30 to 115, 30 to 120, 30 to 125, 30 to 130, 30 to 135, 30 to 140, 30 to 145, 30 to 150, 30 to 155, 30 to 160, 30 to 165, 30 to 170, 30 to 175, 30 to 180, 30 to 185, 30 to 190, 30 to 195, 30 to 200, or more fold of TL1A compared to the corresponding tissue in the control subject. In some embodiments, the diseased tissue in the subject produces 40 to 50, 40 to 55, 40 to 60, 40 to 65, 40 to 70, 40 to 75, 40 to 80, 40 to 85, 40 to 90, 40 to 95, 40 to 100, 40 to 105, 40 to 110, 40 to 115, 40 to 120, 40 to 125, 40 to 130, 40 to 135, 40 to 140, 40 to 145, 40 to 150, 40 to 155, 40 to 160, 40 to 165, 40 to 170, 40 to 175, 40 to 180, 40 to 185, 40 to 190, 40 to 195, 40 to 200, or more fold of TL1A compared to the corresponding tissue in the control subject. In some embodiments, the diseased tissue in the subject produces 50 to 55, 50 to 60, 50 to 65, 50 to 70, 50 to 75, 50 to 80, 50 to 85, 50 to 90, 50 to 95, 50 to 100, 50 to 105, 50 to 110, 50 to 115, 50 to 120, 50 to 125, 50 to 130, 50 to 135, 50 to 140, 50 to 145, 50 to 150, 50 to 155, 50 to 160, 50 to 165, 50 to 170, 50 to 175, 50 to 180, 50 to 185, 50 to 190, 50 to 195, 50 to 200, or more fold of TL1A compared to the corresponding tissue in the control subject. In some embodiments, the diseased tissue in the subject produces 60 to 65, 60 to 70, 60 to 75, 60 to 80, 60 to 85, 60 to 90, 60 to 95, 60 to 100, 60 to 105, 60 to 110, 60 to 115, 60 to 120, 60 to 125, 60 to 130, 60 to 135, 60 to 140, 60 to 145, 60 to 150, 60 to 155, 60 to 160, 60 to 165, 60 to 170, 60 to 175, 60 to 180, 60 to 185, 60 to 190, 60 to 195, 60 to 200, or more fold of TL1A compared to the corresponding tissue in the control subject. In one specific embodiment, the diseased tissue in the subject produces up to or about 50 fold of TL1A compared to the corresponding tissue in the control subject. In another specific embodiment, the diseased tissue in the subject produces up to or about 60 fold of TL1A compared to the corresponding tissue in the control subject. In one specific embodiment, the diseased tissue in the subject produces up to or about 70 fold of TL1A compared to the corresponding tissue in the control subject. In another specific embodiment, the diseased tissue in the subject produces up to or about 80 fold of TL1A compared to the corresponding tissue in the control subject. In one specific embodiment, the diseased tissue in the subject produces up to or about 90 fold of TL1A compared to the corresponding tissue in the control subject. In another specific embodiment, the diseased tissue in the subject produces up to or about 100 fold of TL1A compared to the corresponding tissue in the control subject. In one specific embodiment, the diseased tissue in the subject produces up to or about 110 fold of TL1A compared to the corresponding tissue in the control subject. In another specific embodiment, the diseased tissue in the subject produces up to or about 120 fold of TL1A compared to the corresponding tissue in the control subject. In yet another specific embodiment, the diseased tissue in the subject produces up to or about 130 fold of TL1A compared to the corresponding tissue in the control subject. In a further embodiment, the diseased tissue in the subject produces up to or about 140 fold of TL1A compared to the corresponding tissue in the control subject. In one embodiment, the diseased tissue in the subject produces up to or about 150 fold of TL1A compared to the corresponding tissue in the control subject. In another embodiment, the diseased tissue in the subject produces up to or about 160 fold of TL1A compared to the corresponding tissue in the control subject. In a further embodiment, the diseased tissue in the subject produces up to or about 170 fold of TL1A compared to the corresponding tissue in the control subject. In yet another specific embodiment, the diseased tissue in the subject produces up to or about 180 fold of TL1A compared to the corresponding tissue in the control subject. In one embodiment, the diseased tissue in the subject produces up to or about 190 fold of TL1A compared to the corresponding tissue in the control subject. In another embodiment, the diseased tissue in the subject produces up to or about 200 fold of TL1A compared to the corresponding tissue in the control subject. In some embodiments, the diseased tissue in the subject overproduces TL1A as described in this paragraph during the induction regimen. In some other embodiments, the diseased tissue in the subject overproduces TL1A as described in this paragraph before administering the effective dose. In certain embodiments, the diseased tissue in the subject overproduces TL1A as described in this paragraph within 1, 2, 3, 4, 5, or 6 weeks of start of the induction regimen. As is clear from the description, the diseased tissue can overproduce TL1A via any combination of the fold overproduction, timing, and duration as described herein. As is also clear from the description above, by providing the reductions of TL1A in the diseased tissue in this paragraph with the combination therapy, the disclosure also provides that the effective dose of the TL1A inhibitors in the combination therapy provided herein can cover the TL1A over-production, for the fold overproduction, timing and/or duration, with the effective dose or induction regimen, as described in this paragraph.

The induction regimen can comprise one or more administrations of the TL1A inhibitor to reduce the concentration of TL1A in a diseased tissue in the subject. In one embodiment of the effective dose of the TL1A inhibitors in the combination therapy provided herein, including in this Section (Section 4.6), the induction regimen comprises a one-time administration of the TL1A inhibitor. In some embodiments, the induction regimen comprises a one-time administration of the TL1A inhibitor at about 150 mg/dose. In one embodiment, the induction regimen comprises a one-time administration of the TL1A inhibitor at about 200 mg/dose. In another embodiment, the induction regimen comprises a one-time administration of the TL1A inhibitor at about 250 mg/dose. In a further embodiment, the induction regimen comprises a one-time administration of the TL1A inhibitor at about 300 mg/dose. In yet another embodiment, the induction regimen comprises a one-time administration of the TL1A inhibitor at about 350 mg/dose. In one embodiment, the induction regimen comprises a one-time administration of the TL1A inhibitor at about 400 mg/dose. In another embodiment, the induction regimen comprises a one-time administration of the TL1A inhibitor at about 450 mg/dose. In yet another embodiment, the induction regimen comprises a one-time administration of the TL1A inhibitor at about 500 mg/dose. In one embodiment, the induction regimen comprises a one-time administration of the TL1A inhibitor at about 550 mg/dose. In yet another embodiment, the induction regimen comprises a one-time administration of the TL1A inhibitor at about 600 mg/dose. In another embodiment, the induction regimen comprises a one-time administration of the TL1A inhibitor at about 650 mg/dose. In a further embodiment, the induction regimen comprises a one-time administration of the TL1A inhibitor at about 700 mg/dose. In one embodiment, the induction regimen comprises a one-time administration of the TL1A inhibitor at about 750 mg/dose. In another embodiment, the induction regimen comprises a one-time administration of the TL1A inhibitor at about 800 mg/dose. In one embodiment, the induction regimen comprises a one-time administration of the TL1A inhibitor at about 850 mg/dose. In a further embodiment, the induction regimen comprises a one-time administration of the TL1A inhibitor at about 900 mg/dose. In one embodiment, the induction regimen comprises a one-time administration of the TL1A inhibitor at about 950 mg/dose. In yet another embodiment, the induction regimen comprises a one-time administration of the TL1A inhibitor at about 1000 mg/dose. In yet another embodiment, the induction regimen comprises a one-time administration of the TL1A inhibitor at about 1100 mg/dose. In one embodiment, the induction regimen comprises a one-time administration of the TL1A inhibitor at about 1200 mg/dose. In another embodiment, the induction regimen comprises a one-time administration of the TL1A inhibitor at about 1250 mg/dose. In a further embodiment, the induction regimen comprises a one-time administration of the TL1A inhibitor at about 1300 mg/dose. In yet another embodiment, the induction regimen comprises a one-time administration of the TL1A inhibitor at about 1400 mg/dose. In yet another embodiment, the induction regimen comprises a one-time administration of the TL1A inhibitor at about 1500 mg/dose. In one embodiment, the induction regimen comprises a one-time administration of the TL1A inhibitor at about 1600 mg/dose. In another embodiment, the induction regimen comprises a one-time administration of the TL1A inhibitor at about 1700 mg/dose. In a further embodiment, the induction regimen comprises a one-time administration of the TL1A inhibitor at about 1750 mg/dose. In yet another embodiment, the induction regimen comprises a one-time administration of the TL1A inhibitor at about 1800 mg/dose. In yet another embodiment, the induction regimen comprises a one-time administration of the TL1A inhibitor at about 1900 mg/dose. In one embodiment, the induction regimen comprises a one-time administration of the TL1A inhibitor at about 2000 mg/dose.

Alternatively, the induction regimen can comprise multiple administrations of the TL1A inhibitor. In one embodiment, the induction regimen comprises 2, 3, 4, 5, 6, 7, 8, 9, 10, 11, 12, 13, 14, 15, 16, 17, 18, 19, 20, or more administrations the TL1A inhibitor. In another embodiment, the induction regimen comprises administration of about 2000, 1950, 1900, 1850, 1800, 1750, 1700, 1650, 1600, 1550, 1500, 1450, 1400, 1350, 1300, 1250, 1200, 1150, 1000, 950, 900, 850, 800, 750, 700, 650, 600, 550, 500, 450, 400, 350, 300, 250, 200, or 150 mg/dose. In one embodiment, the induction regimen comprises administration of 200 to 2000, 200 to 1950, 200 to 1900, 200 to 1850, 200 to 1800, 200 to 1750, 200 to 1700, 200 to 1650, 200 to 1600, 200 to 1550, 200 to 1500, 200 to 1450, 200 to 1400, 200 to 1350, 200 to 1300, 200 to 1250, 200 to 1200, 200 to 1150, 200 to 1000, 200 to 950, 200 to 900, 200 to 850, 200 to 800, 200 to 750, 200 to 700, 200 to 650, 200 to 600, 200 to 550, 200 to 500, 200 to 450, 200 to 400, 200 to 350, 200 to 300, or 200 to 250 mg/dose. In one embodiment, the induction regimen comprises administration of 100 to 2000, 100 to 1950, 100 to 1900, 100 to 1850, 100 to 1800, 100 to 1750, 100 to 1700, 100 to 1650, 100 to 1600, 100 to 1550, 100 to 1500, 100 to 1450, 100 to 1400, 100 to 1350, 100 to 1300, 100 to 1250, 100 to 1200, 100 to 1150, 100 to 1000, 100 to 950, 100 to 900, 100 to 850, 100 to 800, 100 to 750, 100 to 700, 100 to 650, 100 to 600, 100 to 550, 100 to 500, 100 to 450, 100 to 400, 100 to 350, 100 to 300, or 100 to 250 mg/dose. In one embodiment, the induction regimen comprises administration of 300 to 2000, 300 to 1950, 300 to 1900, 300 to 1850, 300 to 1800, 300 to 1750, 300 to 1700, 300 to 1650, 300 to 1600, 300 to 1550, 300 to 1500, 300 to 1450, 300 to 1400, 300 to 1350, 300 to 1300, 300 to 1250, 300 to 1200, 300 to 1150, 300 to 1000, 300 to 950, 300 to 900, 300 to 850, 300 to 800, 300 to 750, 300 to 700, 300 to 650, 300 to 600, 300 to 550, 300 to 500, 300 to 450, 300 to 400, or 300 to 350 mg/dose. In yet another embodiment, the induction regimen comprises administration once every 1, 2, 3, 4, 5, 6, 7, or 8 weeks. In a further embodiment, the induction regimen comprises administration once every 1, 2, 3 or 4 weeks for the first 2 administrations and then once every 1, 2, 3, 4, 5, 6, 7, or 8 weeks for the remaining induction regimen. In one embodiment, the induction regimen comprises administration week 0 and week 2 for the first 2 administrations and then once every 1, 2, 3, 4, 5, 6, 7, or 8 weeks for the remaining induction regimen. In another embodiment, the duration of the induction regimen is shorter than the duration of the maintenance regimen. In a further embodiment, the induction regimen continues for 6, 7, 8, 9, 10, 11, 12, 13, 14, 15, 16, 17, 18, 19, 20, or more weeks. The disclosure further provides that the induction regimen can comprise any combination of the dosing amount, dosing frequency, number of administrations, and/or the duration of the induction regimen. Accordingly and as an example, in some embodiments, the induction regimen can comprise administration of about 2000, 1950, 1900, 1850, 1800, 1750, 1700, 1650, 1600, 1550, 1500, 1450, 1400, 1350, 1300, 1250, 1200, 1150, 1000, 950, 900, 850, 800, 750, 700, 650, 600, 550, 500, 450, 400, 350, 300, 250, or 200 mg/dose for administrations at week 0 and week 2 for the first 2 administrations and then once every 2, 3, 4, 5, 6, 7, or 8 weeks, for a duration of 6, 7, 8, 9, 10, 11, 12, 13, 14, 15, 16, 17, 18, 19, 20, or more weeks for the induction regimen. Similarly, in some embodiments, the induction regimen can comprise administration of about 2000, 1950, 1900, 1850, 1800, 1750, 1700, 1650, 1600, 1550, 1500, 1450, 1400, 1350, 1300, 1250, 1200, 1150, 1000, 950, 900, 850, 800, 750, 700, 650, 600, 550, 500, 450, 400, 350, 300, 250, or 200 mg/dose for administrations at week 0 and week 2 for the first 2 administrations and then administration of about 1500, 1450, 1400, 1350, 1300, 1250, 1200, 1150, 1000, 950, 900, 850, 800, 750, 700, 650, 600, 550, 500, 450, 400, 350, 300, 250, 200, or 150 mg/dose once every 2, 3, 4, 5, 6, 7, or 8 weeks, for a duration of 6, 7, 8, 9, 10, 11, 12, 13, 14, 15, 16, 17, 18, 19, 20, or more weeks for the induction regimen.

Specifically, in some embodiments, the induction regimen comprises administrations of about 1000 mg/dose on week 0, about 1000 mg/dose on week 2, about 1000 mg/dose on week 6, and about 1000 mg/dose on week 10. In some embodiments, the induction regimen comprises administrations of about 500 mg/dose on week 0, about 500 mg/dose on week 2, about 500 mg/dose on week 6, and about 500 mg/dose on week 10. In some embodiments, the induction regimen comprises administrations of about 1000 mg/dose on week 0, about 1000 mg/dose on week 2, about 1000 mg/dose on week 6, and about 500 mg/dose on week 10. In some embodiments, the induction regimen comprises administrations of about 1000 mg/dose on week 0, about 1000 mg/dose on week 2, about 500 mg/dose on week 6, and about 500 mg/dose on week 10. In some embodiments, the induction regimen comprises administrations of about 1000 mg/dose on week 0, about 500 mg/dose on week 2, about 500 mg/dose on week 6, and about 500 mg/dose on week 10. In some embodiments, the induction regimen comprises administrations of about 750 mg/dose on week 0, about 750 mg/dose on week 2, about 750 mg/dose on week 6, and about 750 mg/dose on week 10. In some embodiments, the induction regimen comprises administrations of about 500 mg/dose on week 0, about 500 mg/dose on week 2, about 500 mg/dose on week 6, and about 500 mg/dose on week 10. In some embodiments, the induction regimen comprises administrations of about 750 mg/dose on week 0, about 750 mg/dose on week 2, about 750 mg/dose on week 6, and about 500 mg/dose on week 10. In some embodiments, the induction regimen comprises administrations of about 750 mg/dose on week 0, about 750 mg/dose on week 2, about 500 mg/dose on week 6, and about 500 mg/dose on week 10. In some embodiments, the induction regimen comprises administrations of about 750 mg/dose on week 0, about 500 mg/dose on week 2, about 500 mg/dose on week 6, and about 500 mg/dose on week 10. In some embodiments, the induction regimen comprises administrations of about 1500 mg/dose on week 0, about 1500 mg/dose on week 2, about 1500 mg/dose on week 6, and about 1500 mg/dose on week 10. In some embodiments, the induction regimen comprises administrations of about 500 mg/dose on week 0, about 500 mg/dose on week 2, about 500 mg/dose on week 6, and about 500 mg/dose on week 10. In some embodiments, the induction regimen comprises administrations of about 1500 mg/dose on week 0, about 1500 mg/dose on week 2, about 1500 mg/dose on week 6, and about 500 mg/dose on week 10. In some embodiments, the induction regimen comprises administrations of about 1500 mg/dose on week 0, about 1500 mg/dose on week 2, about 500 mg/dose on week 6, and about 500 mg/dose on week 10. In some embodiments, the induction regimen comprises administrations of about 1500 mg/dose on week 0, about 500 mg/dose on week 2, about 500 mg/dose on week 6, and about 500 mg/dose on week 10. In some embodiments, the induction regimen comprises administrations of about 750 mg/dose on week 0, about 750 mg/dose on week 2, about 750 mg/dose on week 6, and about 750 mg/dose on week 10. In some embodiments, the induction regimen comprises administrations of about 1000 mg/dose on week 0, about 1000 mg/dose on week 2, about 1000 mg/dose on week 6, and about 750 mg/dose on week 10. In some embodiments, the induction regimen comprises administrations of about 1000 mg/dose on week 0, about 1000 mg/dose on week 2, about 750 mg/dose on week 6, and about 750 mg/dose on week 10. In some embodiments, the induction regimen comprises administrations of about 1000 mg/dose on week 0, about 750 mg/dose on week 2, about 750 mg/dose on week 6, and about 750 mg/dose on week 10. In some embodiments, the induction regimen comprises administrations of about 1500 mg/dose on week 0, about 1500 mg/dose on week 2, about 1500 mg/dose on week 6, and about 1500 mg/dose on week 10. In some embodiments, the induction regimen comprises administrations of about 750 mg/dose on week 0, about 750 mg/dose on week 2, about 750 mg/dose on week 6, and about 750 mg/dose on week 10. In some embodiments, the induction regimen comprises administrations of about 1500 mg/dose on week 0, about 1500 mg/dose on week 2, about 1500 mg/dose on week 6, and about 750 mg/dose on week 10. In some embodiments, the induction regimen comprises administrations of about 1500 mg/dose on week 0, about 1500 mg/dose on week 2, about 750 mg/dose on week 6, and about 750 mg/dose on week 10. In some embodiments, the induction regimen comprises administrations of about 1500 mg/dose on week 0, about 750 mg/dose on week 2, about 750 mg/dose on week 6, and about 750 mg/dose on week 10.

In one embodiment, the duration of the induction regimen is shorter than the duration of the maintenance regimen. In a further embodiment, the induction regimen continues for 2, 3, 4, 5, 6, 7, 8, 9, 10, 11, 12, 13, 14, 15, or 16 weeks. In another embodiment, the induction regimen continues for 2, 3, 4, 5, 6, 7, 8, 9, 10, 11, or 12 weeks. In yet another embodiment, the induction regimen continues for 8 weeks. In one embodiment, the induction regimen continues for 9 weeks. In one embodiment, the induction regimen continues for 10 weeks. In one embodiment, the induction regimen continues for 11 weeks. In one embodiment, the induction regimen continues for 12 weeks.

As used herein, week 0 means day 1 of the administration of the TL1A inhibitor. Week 0 of the induction regimen means day 1 of the administration of the TL1A inhibitor in the induction regimen. Week 0 of the maintenance regimen means day 1 of the administration of the TL1A inhibitor in the maintenance regimen.

The disclosure provides that the diseased tissue in the subject can overproduce and/or continue to overproduce (e.g. cells in the diseased tissue overexpresses) TL1A after the induction regimen. Thus, in some embodiments, the disclosure further provides a maintenance regimen for the effective dose of the TL1A inhibitors in the combination therapy to maintain the TL1A in the diseased tissue in the subject at a concentration below the concentration of TL1A in the corresponding tissue in the control subject without the inflammatory disease or condition. In certain embodiments, the effective dose of the TL1A inhibitors in the combination therapy further comprise a maintenance regimen to maintain the TL1A in the diseased tissue in the subject at a concentration below the concentration of TL1A in a reference tissue in the control subject without the inflammatory disease or condition. In some other embodiments, the effective dose of the TL1A inhibitors in the combination therapy further comprise a maintenance regimen to maintain the TL1A in the diseased tissue in the subject at a concentration below a reference TL1A level (e.g. a reference concentration).

As described herein, the concentration of TL1A in the diseased tissue of the subject is reduced below (i) a reference TL1A level or (ii) the concentration of TL1A in a corresponding tissue or a reference tissue in in a control subject without the inflammatory disease or condition, while the diseased tissues (e.g. certain cells in the diseased tissues) of the subject overproduces TL1A. Accordingly, the reduction of the TL1A in the diseased tissue can be maintained at or during any or all time of the maintenance regimen, while the diseased tissues (e.g. certain cells in the diseased tissues) of the subject overproduces TL1A at various level of overproduction. In some embodiments of the effective dose of the TL1A inhibitors in the combination therapy provided herein, including in this Section (Section 4.6), the diseased tissue in the subject produces up to 10, up to 15, up to 20, up to 25, up to 30, up to 35, up to 40, up to 45, up to 50, up to 55, up to 60, up to 65, up to 70, up to 75, up to 80, up to 85, up to 90, up to 95, up to 100, or up to more fold of TL1A compared to the corresponding tissue in the control subject during the maintenance regimen. In certain embodiments, the diseased tissue in the subject produces about 10, about 15, about 20, about 25, about 30, about 35, about 40, about 45, about 50, about 50, about 55, about 60, about 65, about 70, about 75, about 80, about 85, about 90, about 95, about 100, or about more fold of TL1A compared to the corresponding tissue in the control subject during the maintenance regimen. In some embodiments, the diseased tissue in the subject produces 10 to 15, 10 to 20, 10 to 25, 10 to 30, 10 to 35, 10 to 40, 10 to 45, 10 to 50, 10 to 50, 10 to 55, 10 to 60, 10 to 65, 10 to 70, 10 to 75, 10 to 80, 10 to 85, 10 to 90, 10 to 95, 10 to 100 fold of TL1A compared to the corresponding tissue in the control subject during the maintenance regimen. In some embodiments, the diseased tissue in the subject produces 20 to 25, 20 to 30, 20 to 35, 20 to 40, 20 to 45, 20 to 50, 20 to 50, 20 to 55, 20 to 60, 20 to 65, 20 to 70, 20 to 75, 20 to 80, 20 to 85, 20 to 90, 20 to 95, 20 to 100 fold of TL1A compared to the corresponding tissue in the control subject during the maintenance regimen. In some embodiments, the diseased tissue in the subject produces 30 to 35, 30 to 40, 30 to 45, 30 to 50, 30 to 50, 30 to 55, 30 to 60, 30 to 65, 30 to 70, 30 to 75, 30 to 80, 30 to 85, 30 to 90, 30 to 95, 30 to 100 fold of TL1A compared to the corresponding tissue in the control subject during the maintenance regimen. In some embodiments, the diseased tissue in the subject produces 40 to 45, 40 to 50, 40 to 50, 40 to 55, 40 to 60, 40 to 65, 40 to 70, 40 to 75, 40 to 80, 40 to 85, 40 to 90, 40 to 95, 40 to 100 fold of TL1A compared to the corresponding tissue in the control subject during the maintenance regimen. In some embodiments, the diseased tissue in the subject produces 50 to 55, 50 to 60, 50 to 65, 50 to 70, 50 to 75, 50 to 80, 50 to 85, 50 to 90, 50 to 95, 50 to 100 fold of TL1A compared to the corresponding tissue in the control subject during the maintenance regimen. In one embodiment, the diseased tissue in the subject produces up to or about 10 fold of TL1A compared to the corresponding tissue in the control subject during the maintenance regimen. In another embodiment, the diseased tissue in the subject produces up to or about 20 fold of TL1A compared to the corresponding tissue in the control subject during the maintenance regimen. In another embodiment, the diseased tissue in the subject produces up to or about 30 fold of TL1A compared to the corresponding tissue in the control subject during the maintenance regimen. In another embodiment, the diseased tissue in the subject produces up to or about 40 fold of TL1A compared to the corresponding tissue in the control subject during the maintenance regimen. In one specific embodiment, the diseased tissue in the subject produces up to or about 50 fold of TL1A compared to the corresponding tissue in the control subject during the maintenance regimen. In another specific embodiment, the diseased tissue in the subject produces up to or about 60 fold of TL1A compared to the corresponding tissue in the control subject during the maintenance regimen. In one specific embodiment, the diseased tissue in the subject produces up to or about 70 fold of TL1A compared to the corresponding tissue in the control subject during the maintenance regimen. In another specific embodiment, the diseased tissue in the subject produces up to or about 80 fold of TL1A compared to the corresponding tissue in the control subject during the maintenance regimen. In one specific embodiment, the diseased tissue in the subject produces up to or about 90 fold of TL1A compared to the corresponding tissue in the control subject during the maintenance regimen. In another specific embodiment, the diseased tissue in the subject produces up to or about 100 fold of TL1A compared to the corresponding tissue in the control subject during the maintenance regimen. In one embodiment, the diseased tissue in the subject produces up to or about 110 fold of TL1A compared to the corresponding tissue in the control subject during the maintenance regimen. In another embodiment, the diseased tissue in the subject produces up to or about 120 fold of TL1A compared to the corresponding tissue in the control subject during the maintenance regimen. As is clear from the description above, by providing the reductions of TL1A in the diseased tissue in this paragraph with the combination therapy, the disclosure also provides that the effective dose of TL1A inhibitors in the combination therapy provided herein can cover the TL1A over-production, for the fold overproduction, timing and/or duration, with the effective dose or maintenance regimen, as described in this paragraph. As is clear from the description above, the diseased tissue in the subject can overproduce TL1A before the maintenance regimen, during the maintenance regimen, or within 1, 2, 3, 4, 5, 6, 7, 8, 9, 10, 11, 12, 14, 16, 18, 20, 22, 24, 28, 32, 36, 40, 44, 48, or 52 weeks of the start of the maintenance regimen. Accordingly, analogous embodiments of the effective dose of the TL1A inhibitors in the combination therapy are also provided as those described in this paragraph in which “during the maintenance regimen” is replaced with “before the maintenance regimen.” Similarly, analogous embodiments of the effective dose of the TL1A inhibitors in the combination therapy are also provided as those described in this paragraph in which “during the maintenance regimen” is replaced with “within 1, 2, 3, 4, 5, 6, 7, 8, 9, 10, 11, 12, 14, 16, 18, 20, 22, 24, 28, 32, 36, 40, 44, 48, or 52 weeks of the start of the maintenance regimen.”

The disclosure provides that the maintenance regimen can include multiple administrations of the TL1A inhibitor. In one embodiment of the effective dose of the TL1A inhibitors in the combination therapy provided herein, including in this Section (Section 4.6), the maintenance regimen comprises 2, 3, 4, 5, 6, 7, 8, 9, 10, 11, 12, 13, 14, 15, 16, 17, 18, 19, 20, or more administrations the TL1A inhibitor. In another embodiment, the maintenance regimen comprises administration of about 2000, 1950, 1900, 1850, 1800, 1750, 1700, 1650, 1600, 1550, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, 200, 150, 100, or 50 mg/dose. In one embodiment, the maintenance regimen comprises administration of about 50 to 1000, 50 to 950, 50 to 900, 50 to 850, 50 to 800, 50 to 750, 50 to 700, 50 to 650, 50 to 600, 50 to 550, 50 to 500, 50 to 450, 50 to 400, 50 to 350, 50 to 300, 50 to 250, 50 to 200, 50 to 150, or 50 to 100 mg/dose. In another embodiment, the maintenance regimen comprises administration of about 100 to 1000, 100 to 950, 100 to 900, 100 to 850, 100 to 800, 100 to 750, 100 to 700, 100 to 650, 100 to 600, 100 to 550, 100 to 500, 100 to 450, 100 to 400, 100 to 350, 100 to 300, 100 to 250, 100 to 200, or 100 to 150 mg/dose. In yet another embodiment, the maintenance regimen comprises administration of about 200 to 1000, 200 to 950, 200 to 900, 200 to 850, 200 to 800, 200 to 750, 200 to 700, 200 to 650, 200 to 600, 200 to 550, 200 to 500, 200 to 450, 200 to 400, 200 to 350, 200 to 300, or 200 to 250 mg/dose. In yet another embodiment, the maintenance regimen comprises administration once every 1, 2, 3, 4, 5, 6, 7, 8, 9, 10, 11, or 12 weeks. In a further embodiment, the maintenance regimen continues for 4, 6, 8, 10, 12, 14, 16, 18, 20, 22, 24, 26, 28, 30, 32, 34, 36, 40, 44, 48, 52, or more weeks. The disclosure further provides that the maintenance regimen can comprise any combination of the dosing amount, dosing frequency, number of administrations, and/or the duration of the induction regimen. Accordingly and as an example, in some embodiments, the induction regimen can comprise administration of about 1000, 950, 900, 850, 800, 750, 700, 650, 600, 550, 500, 450, 400, 350, 300, 250, 200, 150, 100, or 50 mg/dose for administrations at a frequency of once every 1, 2, 3, 4, 5, 6, 7, 8, 9, 10, 11, or 12 weeks, for a duration of 4, 6, 8, 10, 12, 14, 16, 18, 20, 22, 24, 26, 28, 30, 32, 34, 36, 40, 44, 48, 52, or more weeks for the maintenance regimen.

Specifically, in some embodiments of the effective dose of the TL1A inhibitors in the combination therapy provided herein, including in this Section (Section 4.6), the maintenance regimen comprises administrations of the TL1A inhibitor at about 500 mg/dose every 2 weeks. In one embodiment, the maintenance regimen comprises administrations of the TL1A inhibitor at about 450 mg/dose every 2 weeks. In one embodiment, the maintenance regimen comprises administrations of the TL1A inhibitor at about 400 mg/dose every 2 weeks. In one embodiment, the maintenance regimen comprises administrations of the TL1A inhibitor at about 350 mg/dose every 2 weeks. In one embodiment, the maintenance regimen comprises administrations of the TL1A inhibitor at about 300 mg/dose every 2 weeks. In one embodiment, the maintenance regimen comprises administrations of the TL1A inhibitor at about 250 mg/dose every 2 weeks. In one embodiment, the maintenance regimen comprises administrations of the TL1A inhibitor at about 200 mg/dose every 2 weeks. In one embodiment, the maintenance regimen comprises administrations of the TL1A inhibitor at about 150 mg/dose every 2 weeks. In one embodiment, the maintenance regimen comprises administrations of the TL1A inhibitor at about 100 mg/dose every 2 weeks. In one embodiment, the maintenance regimen comprises administrations of the TL1A inhibitor at about 50 mg/dose every 2 weeks. In one embodiment, the maintenance regimen comprises administrations of the TL1A inhibitor at about 500 mg/dose every 4 weeks. In one embodiment, the maintenance regimen comprises administrations of the TL1A inhibitor at about 450 mg/dose every 4 weeks. In one embodiment, the maintenance regimen comprises administrations of the TL1A inhibitor at about 400 mg/dose every 4 weeks. In one embodiment, the maintenance regimen comprises administrations of the TL1A inhibitor at about 350 mg/dose every 4 weeks. In one embodiment, the maintenance regimen comprises administrations of the TL1A inhibitor at about 300 mg/dose every 4 weeks. In one embodiment, the maintenance regimen comprises administrations of the TL1A inhibitor at about 250 mg/dose every 4 weeks. In one embodiment, the maintenance regimen comprises administrations of the TL1A inhibitor at about 200 mg/dose every 4 weeks. In one embodiment, the maintenance regimen comprises administrations of the TL1A inhibitor at about 150 mg/dose every 4 weeks. In one embodiment, the maintenance regimen comprises administrations of the TL1A inhibitor at about 100 mg/dose every 4 weeks. In one embodiment, the maintenance regimen comprises administrations of the TL1A inhibitor at about 50 mg/dose every 4 weeks. In one embodiment, the maintenance regimen comprises administrations of the TL1A inhibitor at about 500 mg/dose every 6 weeks. In one embodiment, the maintenance regimen comprises administrations of the TL1A inhibitor at about 450 mg/dose every 6 weeks. In one embodiment, the maintenance regimen comprises administrations of the TL1A inhibitor at about 400 mg/dose every 6 weeks. In one embodiment, the maintenance regimen comprises administrations of the TL1A inhibitor at about 350 mg/dose every 6 weeks. In one embodiment, the maintenance regimen comprises administrations of the TL1A inhibitor at about 300 mg/dose every 6 weeks. In one embodiment, the maintenance regimen comprises administrations of the TL1A inhibitor at about 250 mg/dose every 6 weeks. In one embodiment, the maintenance regimen comprises administrations of the TL1A inhibitor at about 200 mg/dose every 6 weeks. In one embodiment, the maintenance regimen comprises administrations of the TL1A inhibitor at about 150 mg/dose every 6 weeks. In one embodiment, the maintenance regimen comprises administrations of the TL1A inhibitor at about 100 mg/dose every 6 weeks. In one embodiment, the maintenance regimen comprises administrations of the TL1A inhibitor at about 50 mg/dose every 6 weeks. In one embodiment, the maintenance regimen comprises administrations of the TL1A inhibitor at about 500 mg/dose every 8 weeks. In one embodiment, the maintenance regimen comprises administrations of the TL1A inhibitor at about 450 mg/dose every 8 weeks. In one embodiment, the maintenance regimen comprises administrations of the TL1A inhibitor at about 400 mg/dose every 8 weeks. In one embodiment, the maintenance regimen comprises administrations of the TL1A inhibitor at about 350 mg/dose every 8 weeks. In one embodiment, the maintenance regimen comprises administrations of the TL1A inhibitor at about 300 mg/dose every 8 weeks. In one embodiment, the maintenance regimen comprises administrations of the TL1A inhibitor at about 250 mg/dose every 8 weeks. In one embodiment, the maintenance regimen comprises administrations of the TL1A inhibitor at about 200 mg/dose every 8 weeks. In one embodiment, the maintenance regimen comprises administrations of the TL1A inhibitor at about 150 mg/dose every 8 weeks. In one embodiment, the maintenance regimen comprises administrations of the TL1A inhibitor at about 100 mg/dose every 8 weeks. In one embodiment, the maintenance regimen comprises administrations of the TL1A inhibitor at about 50 mg/dose every 8 weeks. In one embodiment, the maintenance regimen comprises administrations of the TL1A inhibitor at about 500 mg/dose every 10 weeks. In one embodiment, the maintenance regimen comprises administrations of the TL1A inhibitor at about 450 mg/dose every 10 weeks. In one embodiment, the maintenance regimen comprises administrations of the TL1A inhibitor at about 400 mg/dose every 10 weeks. In one embodiment, the maintenance regimen comprises administrations of the TL1A inhibitor at about 350 mg/dose every 10 weeks. In one embodiment, the maintenance regimen comprises administrations of the TL1A inhibitor at about 300 mg/dose every 10 weeks. In one embodiment, the maintenance regimen comprises administrations of the TL1A inhibitor at about 250 mg/dose every 10 weeks. In one embodiment, the maintenance regimen comprises administrations of the TL1A inhibitor at about 200 mg/dose every 10 weeks. In one embodiment, the maintenance regimen comprises administrations of the TL1A inhibitor at about 150 mg/dose every 10 weeks. In one embodiment, the maintenance regimen comprises administrations of the TL1A inhibitor at about 100 mg/dose every 10 weeks. In one embodiment, the maintenance regimen comprises administrations of the TL1A inhibitor at about 50 mg/dose every 10 weeks.

For various embodiments of the effective dose of the TL1A inhibitors in the combination therapy provided herein, including in this Section (Section 4.6), for example those of the preceding paragraphs), further embodiments of the anti-TL1A antibodies, including embodiments with exemplary CDRs, framework sequences, constant region sequences, Fc mutations, variable regions, Fc regions, and other properties are further provided in Section 4.3.1(a); the embodiments for a soluble DR3 protein, a variant of soluble DR3 protein, a soluble DR3 protein fused with Fc, or a variant of soluble DR3 protein fused with Fc are provided in Section 4.3.1(c); assays for screening, testing, and validating the TL1A inhibitors are provided in Section 4.3.3; methods for generating, improving, mutating, cloning, expressing, and isolating the anti-TL1A antibodies are provided in Section 4.4; pharmaceutical compositions for the TL1A inhibitors are described and provided in Section 4.5; further specific and validated embodiments for the anti-TL1A antibodies and the methods of using the same in the combination therapy are provided in Section 5. As such, the disclosure provides the various combinations of the TL1A inhibitor, the pharmaceutical compositions of such TL1A inhibitor, the methods of generating the TL1A inhibitor, the methods of assaying the TL1A inhibitor, the therapeutically effective amount (such as dose and dosing regimen) of the TL1A inhibitor, and the methods of using the anti-TL1A antibodies in the combination therapy.

The disclosure provides that there is advantage of using anti-TL1A antibody or antigen binding fragments that bind to both monomeric TL1A and trimeric TL1A, as neutralizing both monomeric and trimeric TL1A can more efficiently reduce the functional trimeric TL1A in diseased tissue. For various embodiments of combination therapy provided herein, including in Section 4.7, the TL1A inhibitor comprises an antibody or antigen binding fragment binds to both monomeric TL1A and trimeric TL1A. In some embodiments of the combination therapy provided herein, the anti-TL1A antibody or antigen binding fragment blocks binding of TL1A to DR3. In certain embodiments of the combination therapy provided herein, the anti-TL1A antibody or antigen binding fragment binds to both monomeric TL1A and trimeric TL1A and blocks binding of TL1A to DR3.

The disclosure also provides that the anti-TL1A antibody or antigen fragments may neutralize TL1A at various percentage levels for the combination therapy provided herein, including in this Section (Section 4.6) and Section 4.7. In some embodiments of the effective dose of TL1A inhibitors in the combination therapy provided herein, at least or about 60%, 65%, 70%, 75%, 80%, 85%, 86%, 87%, 88%, 89%, 90%, 91%, 92%, 93%, 94%, 95%, 96%, 97%, 98%, or 99% of the monomeric TL1A in the blood of the subject is neutralized (e.g. occupied and blocked for binding with DR3) by the anti-TL1A antibody or antigen binding fragment in the combination therapy. In certain embodiments of the effective dose of TL1A inhibitors in the combination therapy provided herein, at least or about 60%, 65%, 70%, 75%, 80%, 85%, 86%, 87%, 88%, 89%, 90%, 91%, 92%, 93%, 94%, 95%, 96%, 97%, 98%, or 99% of the trimeric TL1A in the blood of the subject is neutralized (e.g. occupied and blocked for binding with DR3) by the anti-TL1A antibody or antigen binding fragment in the combination therapy. In some further embodiments of the effective dose of TL1A inhibitors in the combination therapy provided herein, (i) at least or about 60%, 65%, 70%, 75%, 80%, 85%, 86%, 87%, 88%, 89%, 90%, 91%, 92%, 93%, 94%, 95%, 96%, 97%, 98%, or 99% of the monomeric TL1A and (ii) at least or about 60%, 65%, 70%, 75%, 80%, 85%, 86%, 87%, 88%, 89%, 90%, 91%, 92%, 93%, 94%, 95%, 96%, 97%, 98%, or 99% of the trimeric TL1A in the blood of the subject are neutralized (e.g. occupied and blocked for binding with DR3) by the anti-TL1A antibody or antigen binding fragment in the combination therapy. In certain embodiments of the effective dose of TL1A inhibitors in the combination therapy provided herein, at least or about 90% of the monomeric TL1A in the blood of the subject is neutralized (e.g. occupied and blocked for binding with DR3) by the anti-TL1A antibody or antigen binding fragment in the combination therapy. In certain embodiments of the effective dose of TL1A inhibitors in the combination therapy provided herein, at least or about 90% of the trimeric TL1A in the blood of the subject is neutralized (e.g. occupied and blocked for binding with DR3) by the anti-TL1A antibody or antigen binding fragment in the combination therapy. In some further embodiments of the effective dose of TL1A inhibitors in the combination therapy provided herein, (i) at least or about 90% of the monomeric TL1A and (ii) at least or about 90% of the trimeric TL1A in the blood of the subject are neutralized (e.g. occupied and blocked for binding with DR3) by the anti-TL1A antibody or antigen binding fragment in the combination therapy. In certain embodiments of the effective dose of TL1A inhibitors in the combination therapy provided herein, at least or about 95% of the monomeric TL1A in the blood of the subject is neutralized (e.g. occupied and blocked for binding with DR3) by the anti-TL1A antibody or antigen binding fragment in the combination therapy. In certain embodiments of the effective dose of TL1A inhibitors in the combination therapy provided herein, at least or about 95% of the trimeric TL1A in the blood of the subject is neutralized (e.g. occupied and blocked for binding with DR3) by the anti-TL1A antibody or antigen binding fragment in the combination therapy. In some further embodiments of the effective dose of TL1A inhibitors in the combination therapy provided herein, (i) at least or about 95% of the monomeric TL1A and (ii) at least or about 95% of the trimeric TL1A in the blood of the subject are neutralized (e.g. occupied and blocked for binding with DR3) by the anti-TL1A antibody or antigen binding fragment in the combination therapy. In certain embodiments of the effective dose of TL1A inhibitors in the combination therapy provided herein, at least or about 99% of the monomeric TL1A in the blood of the subject is neutralized (e.g. occupied and blocked for binding with DR3) by the anti-TL1A antibody or antigen binding fragment in the combination therapy. In certain embodiments of the effective dose of TL1A inhibitors in the combination therapy provided herein, at least or about 99% of the trimeric TL1A in the blood of the subject is neutralized (e.g. occupied and blocked for binding with DR3) by the anti-TL1A antibody or antigen binding fragment in the combination therapy. In some further embodiments of the effective dose of TL1A inhibitors in the combination therapy provided herein, (i) at least or about 99% of the monomeric TL1A and (ii) at least or about 99% of the trimeric TL1A in the blood of the subject are neutralized (e.g. occupied and blocked for binding with DR3) by the anti-TL1A antibody or antigen binding fragment in the combination therapy.

The diseased tissue described or referenced in the effective dose of the TL1A inhibitor in the combination therapy provided herein, including in this Section (Section 4.6), can be one or more tissues manifesting pathology from IBD in the subject. In one embodiment, the diseased tissues comprise or consist of colon. In some embodiments, the diseased tissues comprise or consist of small intestine. In certain embodiments, the diseased tissues comprise or consist of rectum. In other embodiments, the diseased tissues comprise or consist of cecum. In yet other embodiments, the diseased tissues comprise or consist of ileum. In another embodiment, the diseased tissues comprise or consist of a fibrotic tissue from IBD. In yet another embodiment, the diseased tissues comprise or consist of other tissues with IBD pathology. In yet another embodiment, the diseased tissues comprise or consist of spleen. In some embodiments, the diseased tissues comprise or consist of other tissues of IBD pathogenesis. In one embodiment, the diseased tissues comprise or consist of colon and small intestine. In some embodiments, the diseased tissues comprise or consist of colon and rectum. In certain embodiments, the diseased tissues comprise or consist of colon and cecum. In other embodiments, the diseased tissues comprise or consist of colon and ileum. In some embodiments, the diseased tissues comprise or consist of colon and a fibrotic tissue from IBD. In other embodiments, the diseased tissues comprise or consist of colon and other tissues with IBD pathology (or of IBD pathogenesis). In further embodiments, the diseased tissues comprise or consist of small intestine and rectum. In one embodiment, the diseased tissues comprise or consist of small intestine and cecum. In some embodiments, the diseased tissues comprise or consist of small intestine and ileum. In certain embodiments, the diseased tissues comprise or consist of small intestine and a fibrotic tissue from IBD. In some embodiments, the diseased tissues comprise or consist of small intestine and other tissues with IBD pathology (or of IBD pathogenesis). In other embodiments, the diseased tissues comprise or consist of rectum and cecum. In yet other embodiments, the diseased tissues comprise or consist of rectum and ileum. In some embodiments, the diseased tissues comprise or consist of rectum and a fibrotic tissue from IBD. In certain embodiments, the diseased tissues comprise or consist of rectum and other tissues with IBD pathology (or of IBD pathogenesis). In one embodiment, the diseased tissues comprise or consist of cecum and ileum. In another embodiment, the diseased tissues comprise or consist of cecum and a fibrotic tissue from IBD. In one embodiment, the diseased tissues comprise or consist of cecum and other tissues with IBD pathology (or of IBD pathogenesis). In some embodiments, the diseased tissues comprise or consist of ileum and a fibrotic tissue from IBD. In certain embodiments, the diseased tissues comprise or consist of ileum and other tissues with IBD pathology (or of IBD pathogenesis). In one embodiment, the diseased tissues comprise or consist of a fibrotic tissue from IBD and other tissues with IBD pathology (or of IBD pathogenesis). In other embodiments, the diseased tissues comprise or consist of colon, small intestine, and rectum. In yet other embodiments, the diseased tissues comprise or consist of colon, small intestine and cecum. In further embodiments, the diseased tissues comprise or consist of colon, small intestine, and ileum. In some embodiments, the diseased tissues comprise or consist of colon, small intestine, and a fibrotic tissue from IBD. In certain embodiments, the diseased tissues comprise or consist of colon, small intestine, and other tissues with IBD pathology (or of IBD pathogenesis). In some embodiments, the diseased tissues comprise or consist of colon, rectum and cecum. In certain embodiments, the diseased tissues comprise or consist of colon, rectum, and ileum. In some embodiments, the diseased tissues comprise or consist of colon, rectum, and a fibrotic tissue from IBD. In other embodiments, the diseased tissues comprise or consist of colon, rectum, and other tissues with IBD pathology (or of IBD pathogenesis). In yet other embodiments, the diseased tissues comprise or consist of colon, cecum and ileum. In some embodiments, the diseased tissues comprise or consist of colon, cecum and a fibrotic tissue from IBD. In other embodiments, the diseased tissues comprise or consist of colon, cecum and other tissues with IBD pathology (or of IBD pathogenesis). In some embodiments, the diseased tissues comprise or consist of colon, ileum and a fibrotic tissue from IBD. In certain embodiments, the diseased tissues comprise or consist of colon, ileum and other tissues with IBD pathology (or of IBD pathogenesis). In other embodiments, the diseased tissues comprise or consist of colon, a fibrotic tissue from IBD and other tissues with IBD pathology (or of IBD pathogenesis). In some embodiments, the diseased tissues comprise or consist of small intestine, rectum and cecum. In certain embodiments, the diseased tissues comprise or consist of small intestine, rectum, and ileum. In some embodiments, the diseased tissues comprise or consist of small intestine, rectum, and a fibrotic tissue from IBD. In certain embodiments, the diseased tissues comprise or consist of small intestine, rectum, and other tissues with IBD pathology (or of IBD pathogenesis). In other embodiments, the diseased tissues comprise or consist of small intestine, cecum and ileum. In yet other embodiments, the diseased tissues comprise or consist of small intestine, cecum and a fibrotic tissue from IBD. In some embodiments, the diseased tissues comprise or consist of small intestine, cecum and other tissues with IBD pathology (or of IBD pathogenesis). In some embodiments, the diseased tissues comprise or consist of small intestine, ileum and a fibrotic tissue from IBD. In some embodiments, the diseased tissues comprise or consist of small intestine, ileum and other tissues with IBD pathology (or of IBD pathogenesis). In some embodiments, the diseased tissues comprise or consist of small intestine, a fibrotic tissue from IBD and other tissues with IBD pathology (or of IBD pathogenesis). In yet other embodiments, the diseased tissues comprise or consist of rectum, cecum and ileum. In other embodiments, the diseased tissues comprise or consist of rectum, cecum and a fibrotic tissue from IBD. In some embodiments, the diseased tissues comprise or consist of rectum, cecum and other tissues with IBD pathology (or of IBD pathogenesis). In certain embodiments, the diseased tissues comprise or consist of rectum, ileum and a fibrotic tissue from IBD. In other embodiments, the diseased tissues comprise or consist of rectum, ileum and other tissues with IBD pathology (or of IBD pathogenesis). In yet other embodiments, the diseased tissues comprise or consist of rectum, a fibrotic tissue from IBD and other tissues with IBD pathology (or of IBD pathogenesis). In some embodiments, the diseased tissues comprise or consist of cecum, ileum and a fibrotic tissue from IBD. In some embodiments, the diseased tissues comprise or consist of cecum, ileum and other tissues with IBD pathology (or of IBD pathogenesis). In some embodiments, the diseased tissues comprise or consist of cecum, a fibrotic tissue from IBD and other tissues with IBD pathology (or of IBD pathogenesis). In some embodiments, the diseased tissues comprise or consist of ileum, a fibrotic tissue from IBD and other tissues with IBD pathology (or of IBD pathogenesis). In other embodiments, the diseased tissues comprise or consist of colon, small intestine, rectum, and cecum. In further embodiments, the diseased tissues comprise or consist of colon, small intestine, rectum, and ileum. In some embodiments, the diseased tissues comprise or consist of colon, small intestine, rectum, and a fibrotic tissue from IBD. In other embodiments, the diseased tissues comprise or consist of colon, small intestine, rectum, and other tissues with IBD pathology (or of IBD pathogenesis). In some embodiments, the diseased tissues comprise or consist of colon, small intestine, cecum, and ileum. In some embodiments, the diseased tissues comprise or consist of colon, small intestine, cecum, and a fibrotic tissue from IBD. In some embodiments, the diseased tissues comprise or consist of colon, small intestine, cecum, and other tissues with IBD pathology (or of IBD pathogenesis). In some embodiments, the diseased tissues comprise or consist of colon, small intestine, ileum, and a fibrotic tissue from IBD. In some embodiments, the diseased tissues comprise or consist of colon, small intestine, ileum, and other tissues with IBD pathology (or of IBD pathogenesis). In some embodiments, the diseased tissues comprise or consist of colon, small intestine, a fibrotic tissue from IBD, and other tissues with IBD pathology (or of IBD pathogenesis). In certain embodiments, the diseased tissues comprise or consist of colon, rectum, cecum, and ileum. In certain embodiments, the diseased tissues comprise or consist of colon, rectum, cecum, and a fibrotic tissue from IBD. In some embodiments, the diseased tissues comprise or consist of colon, rectum, cecum, and other tissues with IBD pathology (or of IBD pathogenesis). In certain embodiments, the diseased tissues comprise or consist of colon, rectum, ileum, and a fibrotic tissue from IBD. In some embodiments, the diseased tissues comprise or consist of colon, rectum, ileum, and other tissues with IBD pathology (or of IBD pathogenesis). In other embodiments, the diseased tissues comprise or consist of colon, rectum, a fibrotic tissue from IBD, and other tissues with IBD pathology (or of IBD pathogenesis). In yet other embodiments, the diseased tissues comprise or consist of colon, cecum, ileum, and a fibrotic tissue from IBD. In some embodiments, the diseased tissues comprise or consist of colon, cecum, ileum, and other tissues with IBD pathology (or of IBD pathogenesis). In certain embodiments, the diseased tissues comprise or consist of colon, cecum, a fibrotic tissue from IBD, and other tissues with IBD pathology (or of IBD pathogenesis). In other embodiments, the diseased tissues comprise or consist of colon, ileum, a fibrotic tissue from IBD, and other tissues with IBD pathology (or of IBD pathogenesis). In further embodiments, the diseased tissues comprise or consist of small intestine, rectum, cecum, and ileum. In some embodiments, the diseased tissues comprise or consist of small intestine, rectum, cecum, and a fibrotic tissue from IBD. In certain embodiments, the diseased tissues comprise or consist of small intestine, rectum, cecum, and other tissues with IBD pathology (or of IBD pathogenesis). In further embodiments, the diseased tissues comprise or consist of small intestine, rectum, ileum, and a fibrotic tissue from IBD. In other embodiments, the diseased tissues comprise or consist of small intestine, rectum, ileum, and other tissues with IBD pathology (or of IBD pathogenesis). In some embodiments, the diseased tissues comprise or consist of small intestine, rectum, a fibrotic tissue from IBD, and other tissues with IBD pathology (or of IBD pathogenesis). In some embodiments, the diseased tissues comprise or consist of small intestine, cecum, ileum, and a fibrotic tissue from IBD. In some embodiments, the diseased tissues comprise or consist of small intestine, cecum, ileum, and other tissues with IBD pathology (or of IBD pathogenesis). In some embodiments, the diseased tissues comprise or consist of small intestine, cecum, a fibrotic tissue from IBD, and other tissues with IBD pathology (or of IBD pathogenesis). In some embodiments, the diseased tissues comprise or consist of small intestine, ileum, a fibrotic tissue from IBD, and other tissues with IBD pathology (or of IBD pathogenesis). In some embodiments, the diseased tissues comprise or consist of rectum, cecum, ileum, and a fibrotic tissue from IBD. In some embodiments, the diseased tissues comprise or consist of rectum, cecum, ileum, and other tissues with IBD pathology (or of IBD pathogenesis). In some embodiments, the diseased tissues comprise or consist of rectum, cecum, a fibrotic tissue from IBD, and other tissues with IBD pathology (or of IBD pathogenesis). In some embodiments, the diseased tissues comprise or consist of rectum, ileum, a fibrotic tissue from IBD, and other tissues with IBD pathology (or of IBD pathogenesis). In some embodiments, the diseased tissues comprise or consist of cecum, ileum, a fibrotic tissue from IBD, and other tissues with IBD pathology (or of IBD pathogenesis). In certain embodiments, the diseased tissues comprise or consist of colon, small intestine, rectum, cecum, and ileum. In some embodiments, the diseased tissues comprise or consist of colon, small intestine, rectum, cecum, and a fibrotic tissue from IBD. In certain embodiments, the diseased tissues comprise or consist of colon, small intestine, rectum, cecum, and other tissues with IBD pathology (or of IBD pathogenesis). In some embodiments, the diseased tissues comprise or consist of colon, small intestine, rectum, ileum, and a fibrotic tissue from IBD. In certain embodiments, the diseased tissues comprise or consist of colon, small intestine, rectum, ileum, and other tissues with IBD pathology (or of IBD pathogenesis). In some embodiments, the diseased tissues comprise or consist of colon, small intestine, rectum, a fibrotic tissue from IBD, and other tissues with IBD pathology (or of IBD pathogenesis). In certain embodiments, the diseased tissues comprise or consist of colon, small intestine, cecum, ileum, and a fibrotic tissue from IBD. In some embodiments, the diseased tissues comprise or consist of colon, small intestine, cecum, ileum, and other tissues with IBD pathology (or of IBD pathogenesis). In certain embodiments, the diseased tissues comprise or consist of colon, small intestine, cecum, a fibrotic tissue from IBD, and other tissues with IBD pathology (or of IBD pathogenesis). In some embodiments, the diseased tissues comprise or consist of colon, small intestine, ileum, a fibrotic tissue from IBD, and other tissues with IBD pathology (or of IBD pathogenesis). In certain embodiments, the diseased tissues comprise or consist of colon, rectum, cecum, ileum, and a fibrotic tissue from IBD. In some embodiments, the diseased tissues comprise or consist of colon, rectum, cecum, ileum, and other tissues with IBD pathology (or of IBD pathogenesis). In certain embodiments, the diseased tissues comprise or consist of colon, rectum, cecum, a fibrotic tissue from IBD, and other tissues with IBD pathology (or of IBD pathogenesis). In some embodiments, the diseased tissues comprise or consist of colon, rectum, ileum, a fibrotic tissue from IBD, and other tissues with IBD pathology (or of IBD pathogenesis). In certain embodiments, the diseased tissues comprise or consist of colon, cecum, ileum, a fibrotic tissue from IBD, and other tissues with IBD pathology (or of IBD pathogenesis). In some embodiments, the diseased tissues comprise or consist of small intestine, rectum, cecum, ileum, and a fibrotic tissue from IBD. In certain embodiments, the diseased tissues comprise or consist of small intestine, rectum, cecum, ileum, and other tissues with IBD pathology (or of IBD pathogenesis). In some embodiments, the diseased tissues comprise or consist of small intestine, rectum, cecum, a fibrotic tissue from IBD, and other tissues with IBD pathology (or of IBD pathogenesis). In some embodiments, the diseased tissues comprise or consist of small intestine, rectum, ileum, a fibrotic tissue from IBD, and other tissues with IBD pathology (or of IBD pathogenesis). In certain embodiments, the diseased tissues comprise or consist of small intestine, cecum, ileum, a fibrotic tissue from IBD, and other tissues with IBD pathology (or of IBD pathogenesis). In some embodiments, the diseased tissues comprise or consist of rectum, cecum, ileum, a fibrotic tissue from IBD, and other tissues with IBD pathology (or of IBD pathogenesis). In some embodiments, the diseased tissues comprise or consist of colon, small intestine, rectum, cecum, ileum, and a fibrotic tissue from IBD. In certain embodiments, the diseased tissues comprise or consist of colon, small intestine, rectum, cecum, ileum, and other tissues with IBD pathology (or of IBD pathogenesis). In some embodiments, the diseased tissues comprise or consist of colon, small intestine, rectum, cecum, a fibrotic tissue from IBD, and other tissues with IBD pathology (or of IBD pathogenesis). In certain embodiments, the diseased tissues comprise or consist of colon, small intestine, rectum, ileum, a fibrotic tissue from IBD, and other tissues with IBD pathology (or of IBD pathogenesis). In some embodiments, the diseased tissues comprise or consist of colon, small intestine, cecum, ileum, a fibrotic tissue from IBD, and other tissues with IBD pathology (or of IBD pathogenesis). In certain embodiments, the diseased tissues comprise or consist of colon, rectum, cecum, ileum, a fibrotic tissue from IBD, and other tissues with IBD pathology (or of IBD pathogenesis). In some embodiments, the diseased tissues comprise or consist of small intestine, rectum, cecum, ileum, a fibrotic tissue from IBD, and other tissues with IBD pathology (or of IBD pathogenesis). In some embodiments, the diseased tissues comprise or consist of any one of colon, small intestine, rectum, cecum, ileum, a fibrotic tissue from IBD, and other tissues with IBD pathology (or of IBD pathogenesis). In some embodiments, the diseased tissues comprise or consist of any two of colon, small intestine, rectum, cecum, ileum, a fibrotic tissue from IBD, and other tissues with IBD pathology (or of IBD pathogenesis), in any combination or permutation. In some embodiments, the diseased tissues comprise or consist of any three of colon, small intestine, rectum, cecum, ileum, a fibrotic tissue from IBD, and other tissues with IBD pathology (or of IBD pathogenesis), in any combination or permutation. In some embodiments, the diseased tissues comprise or consist of any four of colon, small intestine, rectum, cecum, ileum, a fibrotic tissue from IBD, and other tissues with IBD pathology (or of IBD pathogenesis), in any combination or permutation. In some embodiments, the diseased tissues comprise or consist of any five of colon, small intestine, rectum, cecum, ileum, a fibrotic tissue from IBD, and other tissues with IBD pathology (or of IBD pathogenesis), in any combination or permutation. In some embodiments, the diseased tissues comprise or consist of any six of colon, small intestine, rectum, cecum, ileum, a fibrotic tissue from IBD, and other tissues with IBD pathology (or of IBD pathogenesis), in any combination or permutation. In some embodiments, the diseased tissues comprise or consist of all seven of colon, small intestine, rectum, cecum, ileum, a fibrotic tissue from IBD, and other tissues with IBD pathology (or of IBD pathogenesis).

As is clear from the previous paragraph, the diseased tissue can also include spleen. In one embodiment, the diseased tissues comprise or consist of spleen and any one selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of spleen and any two selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of spleen and any three selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of spleen and any four selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of spleen and any five selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of spleen and any six selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of spleen and any seven selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of spleen and all eight selected from the group consisting of colon, small intestine, rectum, cecum, ileum, a fibrotic tissue from IBD, other tissues with IBD pathology, and other tissues of IBD pathogenesis. In one embodiment, the diseased tissues comprise or consist of any one selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of any two selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of any three selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of any four selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of any five selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of any six selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of any seven selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of any eight selected from the group consisting of colon, small intestine, rectum, cecum, ileum, spleen, a fibrotic tissue from IBD, other tissues with IBD pathology, and other tissues of IBD pathogenesis. In one embodiment, the diseased tissues comprise or consist of all nine selected from the group consisting of colon, small intestine, rectum, cecum, ileum, spleen, a fibrotic tissue from IBD, other tissues with IBD pathology, and other tissues of IBD pathogenesis. For clarity, in some embodiments, the diseased tissues comprise or consist of any number of tissues (e.g. one or more), in any combination or permutation, selected from the group consisting of colon, small intestine, rectum, cecum, ileum, spleen, a fibrotic tissue from IBD, other tissues with IBD pathology, and other tissues of IBD pathogenesis.

The tissues with IBD pathology refer to tissues that have manifested changes caused by IBD. Such manifested changes for IBD pathology can be changes in gene or protein expression profile (e.g. higher TL1A expression and/or IFNγ expression), histology changes (e.g. changes in the organization and arrangements of the various cell types (such as damages to layers of epithelial cells), changes in the amount or ratio of cell various cells types (such as loss of certain cells or over-amplification of some cells), and/or occurrence of cell types not normally seen in the tissue (such as infiltration of monocytes in the tissue)).

The tissues of IBD pathogenesis refers to tissues that have manifested changes that will cause or contribute to the development of IBD. Such manifested changes of IBD pathogenesis can be changes in gene or protein expression profile (e.g. higher TL1A expression and/or IFNγ expression), changes in the transportation of proteins or cells (e.g. increased secretion of TL1A and/or IFNγ or increased migration of monocyte to other tissues of IBD pathology), and/or other changes that can cause inflammation in the tissues of IBD pathology. The disclosure provides that the tissues of IBD pathogenesis and the tissues with IBD pathology are not mutually exclusive. Thus certain tissues of IBD pathogenesis can also be tissues with IBD pathology and some tissues with IBD pathology can also be tissues of IBD pathogenesis.

The corresponding tissue provided herein for the effective dose of the TL1A inhibitor in the combination therapy for determining the fold overproduction of TL1A in the diseased tissue can be the same or equivalent tissue as the diseased tissue but in a control subject without the inflammatory disease or condition. For example, when the diseased tissue in an IBD patient is colon, the corresponding tissue can be colon, or one or more parts of colon, tissue close to colon, or tissue whose TL1A level correlates with that in colon. Alternatively, the corresponding tissue provided herein for the effective dose of TL1A inhibitors in the combination therapy for determining the fold overproduction of TL1A in the diseased tissue can be a reference tissue in a control subject without the inflammatory disease or condition. Additionally, the corresponding tissue provided herein for the effective dose of TL1A inhibitors in the combination therapy for determining the fold overproduction of TL1A in the diseased tissue can be a reference tissue that is not affected by the IBD in the same diseased subject. Such reference tissues are not necessarily the same as the diseased tissue, as long as the TL1A concentration in such reference tissue reflects the physiological or basal level of TL1A production as further described in the paragraph below. Such reference tissues in a control subject can be colon, small intestine, rectum, cecum, spleen, ileum, and/or a tissue (or tissues) without IBD pathology or abnormal TL1A expression. In one embodiment, the corresponding tissue or reference tissue in the control subject comprises or consists of colon. In one embodiment, the corresponding tissue or reference tissue in the control subject comprises or consists of small intestine. In one embodiment, the corresponding tissue or reference tissue in the control subject comprises or consists of rectum. In one embodiment, the corresponding tissue or reference tissue in the control subject comprises or consists of cecum. In one embodiment, the corresponding tissue or reference tissue in the control subject comprises or consists of ileum. In one embodiment, the corresponding tissue or reference tissue in the control subject comprises or consists of a tissue (or tissues) without IBD pathology or abnormal TL1A expression. In one embodiment, the corresponding tissue or reference tissue in the control subject comprises or consists of any combination of 2, 3, 4, 5, 6, or more tissues selected from the group consisting of colon, small intestine, rectum, cecum, ileum, spleen, and other tissues without IBD pathology or abnormal TL1A expression. In one embodiment, the corresponding tissue or reference tissue in the control subject comprises or consists of any combination of 2 tissues selected from the group consisting of colon, small intestine, rectum, cecum, ileum, spleen, and a tissue (or tissues) without IBD pathology or abnormal TL1A expression. In one embodiment, the corresponding tissue or reference tissue in the control subject comprises or consists of any combination of 3 tissues selected from the group consisting of colon, small intestine, rectum, cecum, ileum, spleen, and a tissue (or tissues) without IBD pathology or abnormal TL1A expression. In one embodiment, the corresponding tissue or reference tissue in the control subject comprises or consists of any combination of 4 tissues selected from the group consisting of colon, small intestine, rectum, cecum, ileum, spleen, and a tissue (or tissues) without IBD pathology or abnormal TL1A expression. In one embodiment, the corresponding tissue or reference tissue in the control subject comprises or consists of any combination of 5 tissues selected from the group consisting of colon, small intestine, rectum, cecum, ileum, spleen, and a tissue (or tissues) without IBD pathology or abnormal TL1A expression. In one embodiment, the corresponding tissue or reference tissue in the control subject comprises or consists of any combination of 6 tissues selected from the group consisting of colon, small intestine, rectum, cecum, ileum, spleen, and a tissue (or tissues) without IBD pathology or abnormal TL1A expression. In some embodiments of the effective dose of TL1A inhibitors in the combination therapy provided herein, including in this Section (Section 4.6), the fold overproduction of TL1A in the diseased tissue can be determined over a reference level of TL1A instead of over the TL1A level in the corresponding tissue in a control subject without the inflammatory disease or condition. Such reference level of TL1A can be a specific concentration, a specific unit of TL1A protein, and/or a specific proxy measurement of TL1A.

As used herein, the TL1A concentration in the corresponding tissue or the reference tissue used for comparing with a diseased tissue for the TL1A over-production refers to the TL1A concentration in such corresponding tissue or reference tissue at the physiological or basal level of TL1A production under normal healthy conditions, i.e. without IBD or other disease or conditions (e.g. inflammatory or immunodeficient conditions) that increases or suppresses TL1A production. In other words, the corresponding tissue or the reference tissue used herein refer to normal healthy tissues without pathology or stimuli that result in abnormal TL1A production. Such physiological or basal level of TL1A can be the average of TL1A concentrations in the corresponding tissue or the reference tissue during a time period, if the TL1A concentration fluctuates with the normal healthy physiological activity of such tissue during the time period. In some embodiments, the period of time used to average the TL1A concentration can be, 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7 days. The reference tissue is also referred to as the normal reference tissue in some descriptions herein for clarity.

As is clear from the descriptions herein, the subject that is the target for administering the anti-TL1A antibodies or antigen binding fragments in the various combination therapies provided herein can be a subject having IBD. In one embodiment, the subject that is the target for administering the anti-TL1A antibodies or antigen binding fragments in the various combination therapies provided herein is a patient with a diseased tissue (e.g. as described above) from IBD. In another embodiment, the subject that is the target for administering the anti-TL1A antibodies or antigen binding fragments in the various combination therapies provided herein is a human subject. In another embodiment, the subject that is the target for administering the anti-TL1A antibodies or antigen binding fragments in the various combination therapies provided herein is an IBD patient. In a further embodiment, the subject that is the target for administering the anti-TL1A antibodies or antigen binding fragments in the various combination therapies provided herein is a patient with ulcerative colitis. In yet another embodiment, the subject that is the target for administering the anti-TL1A antibodies or antigen binding fragments in the various combination therapies provided herein is a patient with Crohn's disease. In one embodiment, the subject that is the target for administering the anti-TL1A antibodies or antigen binding fragments in the various combination therapies provided herein is a patient with both ulcerative colitis and Crohn's disease.

The disclosure provides that the effective dose of TL1A inhibitors provided herein for the combination therapies, including in this Section (Section 4.6), can be determined by a dose determination methods as further described in this Section (Section 4.6, including the below paragraphs). Thus in various aspects and embodiments, provided herein is a method for determining the effective dose of TL1A inhibitors in the combination therapy, including the induction regimen, the maintenance regimen, and both the induction regimen and the maintenance regimen.

In one aspect, provided herein is a method of determining an effective dose regimen for administering an anti-TL1A antibody, wherein the method comprises: (a) receiving association rate of the antibody to monomeric TL1A (kₒₙ₋ₘₒₙₒₘₑᵣ), association rate of the antibody to trimeric TL1A (kₒₙ₋ₜᵣᵢₘₑᵣ), dissociation rate of the antibody from monomeric TL1A (k_{off-monomer}), dissociation rate of the antibody from trimeric TL1A (k_{off-trimer}), synthesis rate of TL1A in normal tissue (k_{syn-normal}), synthesis rate of TL1A in diseased tissue (k_{syn-disease}), degradation rate of monomeric TL1A (k_{deg-monomer}), and degradation rate of trimeric TL1A (k_{deg-trimer}); (b) integrating the rates received in (a) to an integrated whole-body physiologically based pharmacokinetic (PBPK) model; and (c) determining the effective dose regimen of the anti-TL1A antibody with the PBPK model from (b) such that after administration of the effective dose regimen the concentration of TL1A in a diseased tissue in the subject is below the concentration of TL1A in a corresponding tissue in a control subject without the inflammatory disease or condition.

In another aspect, provided herein is a method of determining an effective dose regimen for administering an anti-TL1A antibody, wherein the method comprises: (a) receiving association rate of the antibody to monomeric TL1A (kₒₙ₋ₘₒₙₒₘₑᵣ), association rate of the antibody to trimeric TL1A (kₒₙ₋ₜᵣᵢₘₑᵣ), dissociation rate of the antibody from monomeric TL1A (k_{off-monomer}), dissociation rate of the antibody from trimeric TL1A (k_{off-trimer}), synthesis rate of TL1A in normal tissue (k_{syn-normal}), synthesis rate of TL1A in diseased tissue (k_{syn-disease}), degradation rate of monomeric TL1A (k_{deg-monomer}), and degradation rate of trimeric TL1A (k_{deg-trimer}); integrating the rates received in (a) to a population pharmacokinetic (popPK) model; and determining the effective dose regimen of the anti-TL1A antibody with the popPK model from (b) such that after administration of the effective dose regimen the concentration of TL1A in a diseased tissue in the subject is below the concentration of TL1A in a corresponding tissue in a control subject without the inflammatory disease or condition.

In a further aspect, provided herein is a method of determining an effective dose regimen for administering an anti-TL1A antibody to a diseased subject, wherein the method comprises: (a) receiving a parameter of TL1A over-production in the diseased tissue comparing to TL1A production in a normal reference tissue; (b) integrating the parameter received in (a) to an integrated whole-body physiologically based pharmacokinetic (PBPK) model; and (c) determining the effective dose regimen of the anti-TL1A antibody with the PBPK model from (b) such that after administration of the effective dose regimen the concentration of TL1A in a diseased tissue in the subject is below the concentration of TL1A in a corresponding tissue in a control subject without the inflammatory disease or condition. In one embodiment of the methods of this paragraph, the diseased subject has an inflammatory disease or condition.

In yet another aspect, provided herein is a method of determining an effective dose regimen for administering an anti-TL1A antibody to a diseased subject, wherein the method comprises: (a) receiving a parameter of TL1A over-production in the diseased tissue comparing to TL1A production in a normal reference tissue; (b) integrating the parameter received in (a) to a population pharmacokinetic (popPK) model; and (c) determining the effective dose regimen of the anti-TL1A antibody with the popPK model from (b) such that after administration of the effective dose regimen the concentration of TL1A in a diseased tissue in the subject is below the concentration of TL1A in a corresponding tissue in a control subject without the inflammatory disease or condition. In one embodiment of the methods of this paragraph, the diseased subject has an inflammatory disease or condition.

The parameter of TL1A over-production in the dose determination methods reflects the over-production of TL1A in the diseased tissues in affected patients, e.g. UC or CD patients. In some embodiments, the parameter of TL1A over-production is 10, 15, 20, 25, 30, 35, 40, 45, 50, 55, 60, 65, 70, 75, 80, 85, 90, 95, 100, 110, 120, 130, 140, 150, 160, 170, 180, 190, 200 or more fold over-production comparing to TL1A production in the normal reference tissue. In certain embodiments, the parameter of TL1A over-production can be various percentages or folds reflecting the over-production of TL1A in the diseased tissues in affected patients, e.g. UC or CD patients. In one embodiment, the parameter of TL1A over-production is up to or about 5 fold over-production comparing to TL1A production in the normal reference tissue. In one embodiment, the parameter of TL1A over-production is up to or about 10 fold over-production comparing to TL1A production in the normal reference tissue. In one embodiment, the parameter of TL1A over-production is up to or about 15 fold over-production comparing to TL1A production in the normal reference tissue. In one embodiment, the parameter of TL1A over-production is up to or about 20 fold over-production comparing to TL1A production in the normal reference tissue. In one embodiment, the parameter of TL1A over-production is up to or about 25 fold over-production comparing to TL1A production in the normal reference tissue. In one embodiment, the parameter of TL1A over-production is up to or about 30 fold over-production comparing to TL1A production in the normal reference tissue. In one embodiment, the parameter of TL1A over-production is up to or about 35 fold over-production comparing to TL1A production in the normal reference tissue. In one embodiment, the parameter of TL1A over-production is up to or about 40 fold over-production comparing to TL1A production in the normal reference tissue. In one embodiment, the parameter of TL1A over-production is up to or about 45 fold over-production comparing to TL1A production in the normal reference tissue. In one embodiment, the parameter of TL1A over-production is up to or about 50 fold over-production comparing to TL1A production in the normal reference tissue. In one embodiment, the parameter of TL1A over-production is up to or about 55 fold over-production comparing to TL1A production in the normal reference tissue. In one embodiment, the parameter of TL1A over-production is up to or about 60 fold over-production comparing to TL1A production in the normal reference tissue. In one embodiment, the parameter of TL1A over-production is up to or about 65 fold over-production comparing to TL1A production in the normal reference tissue. In one embodiment, the parameter of TL1A over-production is up to or about 70 fold over-production comparing to TL1A production in the normal reference tissue. In one embodiment, the parameter of TL1A over-production is up to or about 75 fold over-production comparing to TL1A production in the normal reference tissue. In one embodiment, the parameter of TL1A over-production is up to or about 80 fold over-production comparing to TL1A production in the normal reference tissue. In one embodiment, the parameter of TL1A over-production is up to or about 85 fold over-production comparing to TL1A production in the normal reference tissue. In one embodiment, the parameter of TL1A over-production is up to or about 90 fold over-production comparing to TL1A production in the normal reference tissue. In one embodiment, the parameter of TL1A over-production is up to or about 95 fold over-production comparing to TL1A production in the normal reference tissue. In one embodiment, the parameter of TL1A over-production is up to or about 100 fold over-production comparing to TL1A production in the normal reference tissue. In one embodiment, the parameter of TL1A over-production is up to or about 110 fold over-production comparing to TL1A production in the normal reference tissue. In one embodiment, the parameter of TL1A over-production is up to or about 120 fold over-production comparing to TL1A production in the normal reference tissue. In one embodiment, the parameter of TL1A over-production is up to or about 130 fold over-production comparing to TL1A production in the normal reference tissue. In one embodiment, the parameter of TL1A over-production is up to or about 140 fold over-production comparing to TL1A production in the normal reference tissue. In one embodiment, the parameter of TL1A over-production is up to or about 150 fold over-production comparing to TL1A production in the normal reference tissue. In one embodiment, the parameter of TL1A over-production is up to or about 160 fold over-production comparing to TL1A production in the normal reference tissue. In one embodiment, the parameter of TL1A over-production is up to or about 170 fold over-production comparing to TL1A production in the normal reference tissue. In one embodiment, the parameter of TL1A over-production is up to or about 180 fold over-production comparing to TL1A production in the normal reference tissue. In one embodiment, the parameter of TL1A over-production is up to or about 190 fold over-production comparing to TL1A production in the normal reference tissue. In one embodiment, the parameter of TL1A over-production is up to or about 200 fold over-production comparing to TL1A production in the normal reference tissue.

The step (a) in the dose determination methods provided herein including in this Section (Section 4.6) can receive additional parameters, such as the rate of association and dissociation between the anti-TL1A antibodies and TL1A. In one embodiment of the method step (a) further comprises receiving association rate of the antibody to TL1A (k_{on-mAb}), dissociation rate of the antibody from TL1A (k_{off-mAb}), synthesis rate of TL1A in normal tissue (k_{syn-normal}), synthesis rate of TL1A in diseased tissue (k_{syn-disease}), and/or degradation rate of TL1A (k_{deg-total-TL1A}). In one embodiment, the association rate of the antibody to TL1A (k_{on-mAb}) comprises the association rate of the antibody to monomeric TL1A (kₒₙ₋ₘₒₙₒₘₑᵣ) and association rate of the antibody to trimeric TL1A (kₒₙ₋ₜᵣᵢₘₑᵣ). In one embodiment, the dissociation rate of the antibody from TL1A (k_{off-mAb}) comprises the dissociation rate of the antibody from monomeric TL1A (k_{off-monomer}) and dissociation rate of the antibody from trimeric TL1A (k_{off-trimer}). In one embodiment, the degradation rate of TL1A (k_{deg-total-TL1A}) comprises degradation rate of monomeric TL1A (k_{deg-TL1A-monomer}) and degradation rate of trimeric TL1A (k_{deg-TL1A-trimer}). In one embodiment, the association rate of the antibody to TL1A (k_{on-mAb}) comprises the association rate of the antibody to monomeric TL1A (kₒₙ₋ₘₒₙₒₘₑᵣ) and association rate of the antibody to trimeric TL1A (kₒₙ₋ₜᵣᵢₘₑᵣ), and the dissociation rate of the antibody from TL1A (k_{off-mAb}) comprises the dissociation rate of the antibody from monomeric TL1A (k_{off-monomer}) and dissociation rate of the antibody from trimeric TL1A (k_{off-trimer}). In one embodiment, the association rate of the antibody to TL1A (k_{on-mAb}) comprises the association rate of the antibody to monomeric TL1A (kₒₙ₋ₘₒₙₒₘₑᵣ) and association rate of the antibody to trimeric TL1A (kₒₙ₋ₜᵣᵢₘₑᵣ), and the degradation rate of TL1A (k_{deg-total-TL1A}) comprises degradation rate of monomeric TL1A (k_{deg-TL1A-monomer}) and degradation rate of trimeric TL1A (k_{deg-TL1A-trimer}). In one embodiment, the dissociation rate of the antibody from TL1A (k_{off-mAb}) comprises the dissociation rate of the antibody from monomeric TL1A (k_{off-monomer}) and dissociation rate of the antibody from trimeric TL1A (k_{off-trimer}), and the degradation rate of TL1A (k_{deg-total-TL1A}) comprises degradation rate of monomeric TL1A (k_{deg-TL1A-monomer}) and degradation rate of trimeric TL1A (k_{deg-TL1A-trimer}). In one embodiment, the association rate of the antibody to TL1A (k_{on-mAb}) comprises the association rate of the antibody to monomeric TL1A (kₒₙ₋ₘₒₙₒₘₑᵣ) and association rate of the antibody to trimeric TL1A (kₒₙ₋ₜᵣᵢₘₑᵣ), the dissociation rate of the antibody from TL1A (k_{off-mAb}) comprises the dissociation rate of the antibody from monomeric TL1A (k_{off-monomer}) and dissociation rate of the antibody from trimeric TL1A (k_{off-trimer}), and/or the degradation rate of TL1A (k_{deg-total-TL1A}) comprises degradation rate of monomeric TL1A (k_{deg-TL1A-monomer}) and degradation rate of trimeric TL1A (k_{deg-TL1A-trimer}).

Additionally, the dose determination methods can include additional parameters of the anti-TL1A antibody binding to proteins other than the TL1A ligand, such as the parameters of the anti-TL1A antibodies or antigen binding fragments binding to FcRn. In some embodiments, the step (a) of the dose determination methods further comprises receiving association rate of the antibody to FcRn receptor (k_{on-mAb-FcRn}), dissociation rate of the antibody from FcRn (k_{off-mAb-FcRn}), association rate of the antibody-monomeric-TL1A complex to FcRn receptor (k_{on-(mAb-monoTL1A)-FcRn}), dissociation rate of the antibody-monomeric-TL1A complex from FcRn (K_{off-(mAb-monoTL1A)-FcRn}), association rate of the antibody-trimeric-TL1A complex to FcRn receptor (k_{on-(mAb-triTL1A)-FcRn}), and/or dissociation rate of the antibody-trimeric-TL1A complex from FcRn (k_{off-(mAb-triTL1A)-FcRn}). In one embodiment, the step (a) of the dose determination methods further comprises receiving association rate of the antibody to FcRn receptor (k_{on-mAb-FcRn}), and/or dissociation rate of the antibody from FcRn (k_{off-mAb-FcRn}). In another embodiment, the step (a) of the dose determination methods further comprises receiving association rate of the antibody-monomeric-TL1A complex to FcRn receptor (k_{on-(mAb-monoTL1A)-FcRn}), and/or dissociation rate of the antibody-monomeric-TL1A complex from FcRn (k_{off-(mAb-monoTL1A)-FcRn}). In yet another embodiment, the step (a) of the dose determination methods further comprises receiving association rate of the antibody-trimeric-TL1A complex to FcRn receptor (k_{on-(mAb-triTL1A) FcRn}), and/or dissociation rate of the antibody-trimeric-TL1A complex from FcRn (k_{off-(mAb-triTL1A)-FcRn}). In a further embodiment, the step (a) of the dose determination methods further comprises receiving association rate of the antibody-monomeric-TL1A complex to FcRn receptor (k_{on-(mAb-monoTL1A)-FcRn}), dissociation rate of the antibody-monomeric-TL1A complex from FcRn (k_{off-(mAb-monoTL1A)-FcRn}), association rate of the antibody-trimeric-TL1A complex to FcRn receptor (k_{on-(mAb-triTL1A)-FcRn}), and/or dissociation rate of the antibody-trimeric-TL1A complex from FcRn (k_{off-(mAb-triTL1A)-FcRn}).

Alternatively, in some embodiments, the step (a) of the dose determination methods further comprises receiving association rate of the antibody to FcRn receptor (k_{on-mAb-FcRn}), dissociation rate of the antibody from FcRn (k_{off-mAb-FcRn}), association rate of the antibody-TL1A complex to FcRn receptor (k_{on-(mAb-TL1A)-FcRn}), and/or dissociation rate of the antibody-TL1A complex from FcRn (k_{off-(mAb-TL1A)-FcRn}). In one embodiment, the association rate of the antibody-TL1A complex to FcRn receptor (k_{on-(mAb-TL1A)-FcRn}) comprises association rate of the antibody-monomeric-TL1A complex to FcRn receptor (k_{on-(mAb-monoTL1A)-FcRn}) and association rate of the antibody-trimeric-TL1A complex to FcRn receptor (K_{on-(mAb-triTL1A)-FcRn}). In one embodiment, the dissociation rate of the antibody-TL1A complex from FcRn (k_{off-(mAb-TL1A)-FcRn}) comprises dissociation rate of the antibody-monomeric-TL1A complex from FcRn (k_{off-(mAb-monoTL1A)-FcRn}) and dissociation rate of the antibody-trimeric-TL1A complex from FcRn (k_{off-(mAb-triTL1A)-FcRn}). In another embodiment, the association rate of the antibody-TL1A complex to FcRn receptor (k_{on-(mAb-TL1A)-FcRn}) comprises association rate of the antibody-monomeric-TL1A complex to FcRn receptor (k_{on-(mAb-monoTL1A)-FcRn}) and association rate of the antibody-trimeric-TL1A complex to FcRn receptor (k_{on-(mAb-triTL1A)-FcRn}), and/or wherein the dissociation rate of the antibody-TL1A complex from FcRn (k_{off-(mAb-TL1A)-FcRn}) comprises dissociation rate of the antibody-monomeric-TL1A complex from FcRn (k_{off-(mAb-monoTL1A)-FcRn}) and dissociation rate of the antibody-trimeric-TL1A complex from FcRn (k_{off-(mAb-triTL1A)-FcRn}).

Similarly, the dose determination methods can include additional parameters such as the parameters of degradation rate of the complex between the anti-TL1A antibodies or antigen binding fragments and FcRn. In one embodiment, the step (a) of the dose determination methods further comprises receiving clearance rate of FcRn receptor bound by the antibody (k_{deg-mAb-FcRn}). In one embodiment, the clearance rate of FcRn receptor bound by the antibody (k_{deg-mAb-FcRn}) further comprises clearance rate of the antibody to FcRn bound by the antibody-monomeric-TL1A complex (k_{deg-(mAb-monoTL1A)-FcRn}) and clearance rate of FcRn receptor bound by the antibody-trimeric-TL1A complex (k_{deg-(mAb-triTL1A)-FcRn}).

Alternatively, in one embodiment, the step (a) of the dose determination methods further comprises receiving clearance rate of FcRn receptor bound by the antibody (k_{deg-mAb-FcRn}), clearance rate of the antibody to FcRn bound by the antibody-monomeric-TL1A complex (k_{deg-(mAb-monoTL1A)-FcRn}), and/or clearance rate of FcRn receptor bound by the antibody-trimeric-TL1A complex (k_{deg-(mAb-triTL1A)-FcRn}). In one embodiment, the step (a) of the dose determination methods further comprises receiving clearance rate of FcRn receptor bound by the antibody (k_{deg-mAb-FcRn}). In one embodiment, the step (a) of the dose determination methods further comprises receiving clearance rate of the antibody to FcRn bound by the antibody-monomeric-TL1A complex (k_{deg-(mAb-monoTL1A)-FcRn}). In one embodiment, the step (a) of the dose determination methods further comprises receiving clearance rate of FcRn receptor bound by the antibody-trimeric-TL1A complex (k_{deg-(mAb-triTL1A)-FcRn}). In one embodiment, the step (a) of the dose determination methods further comprises receiving clearance rate of FcRn receptor bound by the antibody (k_{deg-mAb-FcRn}) and clearance rate of the antibody to FcRn bound by the antibody-monomeric-TL1A complex (k_{deg-(mAb-monoTL1A)-FcRn}). In one embodiment, the step (a) of the dose determination methods further comprises receiving clearance rate of FcRn receptor bound by the antibody (k_{deg-mAb-FcRn}) and clearance rate of FcRn receptor bound by the antibody-trimeric-TL1A complex (k_{deg-(mAb-triTL1A)-FcRn}). In one embodiment, the step (a) of the dose determination methods further comprises receiving clearance rate of the antibody to FcRn bound by the antibody-monomeric-TL1A complex (k_{deg-(mAb-monoTL1A)-FcRn}) and clearance rate of FcRn receptor bound by the antibody-trimeric-TL1A complex (k_{deg-(mAb-triTL1A)-FcRn}). In one embodiment, the step (a) of the dose determination methods further comprises receiving clearance rate of FcRn receptor bound by the antibody (k_{deg-mAb-FcRn}), clearance rate of the antibody to FcRn bound by the antibody-monomeric-TL1A complex (k_{deg-(mAb-monoTL1A)-FcRn}), and clearance rate of FcRn receptor bound by the antibody-trimeric-TL1A complex (k_{deg-(mAb-triTL1A)-FcRn}).

In addition, in various embodiments of the dose determination methods provided herein, including in this Section (Section 4.6), the step (a) in the dose determination methods further comprises receiving the rate of TL1A trimerization (k_{on-TL1A-monomer-to-trimer}) and/or the rate of TL1A monomerization (k_{off-TL1A-trimer-to-monomer}). In one embodiment, the step (a) in the dose determination methods further comprises receiving the rate of TL1A trimerization (k_{on-TL1A-monomer-to-trimer}). In another embodiment, the step (a) in the dose determination methods further comprises receiving the rate of TL1A monomerization (k_{off-TL1A-trimer-to-monomer}). In yet another embodiment, the step (a) in the dose determination methods further comprises receiving the rate of TL1A trimerization (k_{on-TL1A-monomer-to-trimer}) and the rate of TL1A monomerization (k_{off-TL1A-trimer-to-monomer}).

The term rate of TL1A trimerization refers to the kinetic rate at which TL1A monomers self-associate to form TL1A trimer. The term rate of TL1A monomerization refers to the kinetic rate at which TL1A trimer dissociates into TL1A monomers.

The various parameters in the dose determination methods can be identical or different. The various parameters in the dose determination methods can also be related by a range, a fold difference in value, and/or by a specific difference in value. In one embodiment of the various dose determination methods provided herein, kₒₙ₋ₘₒₙₒₘₑᵣ and kₒₙ₋ₜᵣᵢₘₑᵣ are identical or different. In one embodiment of the various dose determination methods provided herein, k_{off-monomer} and k_{off-trimer} are identical or different. In one embodiment of the various dose determination methods provided herein, k deg-monomer and k deg-trimer are identical or different. In one embodiment of the various dose determination methods provided herein, k_{on-(mAb-monoTL1A)-FcRn} and k_{on-(mAb-triTL1A)-FcRn} are identical or different. In one embodiment of the various dose determination methods provided herein, k_{on-mAb-FcRn} and k_{on-(mAb-monoTL1A)-FcRn} are identical or different. In one embodiment of the various dose determination methods provided herein, k_{on-mAb-FcRn} and k_{on-(mAb-triTILA)-FcRn} are identical or different. In one embodiment of the various dose determination methods provided herein, k_{off-(mAb-monoTL1A) FcRn} and k_{off-(mAb-triTL1A)-FcRn} are identical or different. In one embodiment of the various dose determination methods provided herein, k_{off-mAb-FcRn} and k_{off-(mAb-monoTL1A)-FcRn} are identical or different. In one embodiment of the various dose determination methods provided herein, k_{off-mAb-FcRn} and k_{off-(mAb-triTL1A)-FcRn} are identical or different. In one embodiment of the various dose determination methods provided herein, k_{deg-(mAb-monoTL1A)-FcRn} and k_{deg-(mAb-triTL1A)-FcRn} are identical or different. In one embodiment of the various dose determination methods provided herein, k_{deg-mAb-FcRn} and k_{deg-(mAb-triTL1A)-FcRn} are identical or different. In one embodiment of the various dose determination methods provided herein, k_{deg-mAb-FcRn} and k_{deg-(mAb-monoTL1A)-FcRn} are identical or different. In one embodiment of the various dose determination methods provided herein, the parameters received in the dose determination methods can have any combination of the relationship as described herein, including in this paragraph.

As is clear from the description herein, the diseased tissue overproduces TL1A than a normal tissue. As already provided above, the diseased tissue overproduces TL1A comparing to normal reference tissue and the parameter of TL1A over-production can be 10, 15, 20, 25, 30, 35, 40, 45, 50, 55, 60, 65, 70, 75, 80, 85, 90, 95, 100, 110, 120, 130, 140, 150, 160, 170, 180, 190, 200 or more fold over-production comparing to TL1A production in the normal reference tissue. Therefore, the k_{syn-disease} can be higher than k_{syn-normal} by various percentages or folds. In one embodiment of the dose determination methods, k_{syn-disease} is up to or about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more fold of k_{syn-normal}. In one embodiment of the dose determination methods, k_{syn-disease} is up to or about 5 fold of k_{syn-normal}. In one embodiment of the dose determination methods, k_{syn-disease} is up to or about 10 fold of k_{syn-normal}. In one embodiment of the dose determination methods, k_{syn-disease} is up to or about 15 fold of k_{syn-normal}. In one embodiment of the dose determination methods, k_{syn-disease} is up to or about 20 fold of k_{syn-normal}. In one embodiment of the dose determination methods, k_{syn-disease} is up to or about 25 fold of k_{syn-normal}. In one embodiment of the dose determination methods, k_{syn-disease} is up to or about 30 fold of k_{syn-normal}. In one embodiment of the dose determination methods, k_{syn-disease} is up to or about 35 fold of k_{syn-normal}. In one embodiment of the dose determination methods, k_{syn-disease} is up to or about 40 fold of k_{syn-normal}. In one embodiment of the dose determination methods, k_{syn-disease} is up to or about 45 fold of k_{syn-normal}. In one embodiment of the dose determination methods, k_{syn-disease} is up to or about 50 fold of k_{syn-normal}. In one embodiment of the dose determination methods, k_{syn-disease} is up to or about 55 fold of k_{syn-normal}. In one embodiment of the dose determination methods, k_{syn-disease} is up to or about 60 fold of k_{syn-normal}. In one embodiment of the dose determination methods, k_{syn-disease} is up to or about 65 fold of k_{syn-normal}. In one embodiment of the dose determination methods, k_{syn-disease} is up to or about 70 fold of k_{syn-normal}. In one embodiment of the dose determination methods, k_{syn-disease} is up to or about 75 fold of k_{syn-normal}. In one embodiment of the dose determination methods, k_{syn-disease} is up to or about 80 fold of k_{syn-normal}. In one embodiment of the dose determination methods, k_{syn-disease} is up to or about 85 fold of k_{syn-normal}. In one embodiment of the dose determination methods, k_{syn-disease} is up to or about 90 fold of k_{syn-normal}. In one embodiment of the dose determination methods, k_{syn-disease} is up to or about 95 fold of k_{syn-normal}. In one embodiment of the dose determination methods, k_{syn-disease} is up to or about 100 fold of k_{syn-normal}. In one embodiment of the dose determination methods, k_{syn-disease} is up to or about 110 fold of k_{syn-normal}. In one embodiment of the dose determination methods, k_{syn-disease} is up to or about 120 fold of k_{syn-normal}. In one embodiment of the dose determination methods, k_{syn-disease} is up to or about 130 fold of k_{syn-normal}. In one embodiment of the dose determination methods, k_{syn-disease} is up to or about 140 fold of k_{syn-normal}. In one embodiment of the dose determination methods, k_{syn-disease} is up to or about 150 fold of k_{syn-normal}. In one embodiment of the dose determination methods, k_{syn-disease} is up to or about 160 fold of k_{syn-normal}. In one embodiment of the dose determination methods, k_{syn-disease} is up to or about 170 fold of k_{syn-normal}. In one embodiment of the dose determination methods, k_{syn-disease} is up to or about 180 fold of k_{syn-normal}. In one embodiment of the dose determination methods, k_{syn-disease} is up to or about 190 fold of k_{syn-normal}. In one embodiment of the dose determination methods, k_{syn-disease} is up to or about 200 fold of k_{syn-normal}.

Normal tissue, reference tissue, or normal reference tissue in the methods (including the methods provided in this Section (Section 4.6), such as methods of use/treatment and/or dose determination methods) refers to a tissue without the pathology from IBD and/or without abnormal TL1A expression. In some embodiments of the dose determination methods, such normal tissue comprises or consists of a healthy tissue (e.g. tissue without IBD-related pathology and/or without abnormal TL1A expression) from the subject with the inflammatory disease or condition. In certain embodiments of the dose determination methods, such normal tissue comprises or consists of a corresponding or reference tissue from a subject without the inflammatory disease or condition, as already provided and described in further details in this Section (Section 4.6).

The various parameters for whole-body Physiologically Based Pharmacokinetic (“PBPK”) in the dose determination methods, including the various rate parameters, can be such parameters already known and used in whole-body PBPK, for example as described in Jones H et al., American Association of Pharmaceutical Scientists Journal (AAPS J.) 2013 April; 15 (2): 377-87; Dostalek, M et al., Clin Pharmacokinet, 2013 February; 52 (2): 83-124; Li L et al., AAPS J. 2014 September; 16 (5): 1097-109; Nestorov I. Clin Pharmacokinet. 2003; 42 (10): 883-908. In some embodiments, the various whole-body PBPK parameters in the dose termination methods, including the various rate parameters described in this Section (Section 4.6), can have the value as described in Section 5. In other embodiments, the various whole-body PBPK parameters in the dose termination methods, including the various rate parameters described in this Section (Section 4.6), can be determined as described in Section 5.

Alternatively, the various parameters for Population Pharmacokinetic (“popPK”) model in the dose determination methods, including the various rate parameters, can be such parameters already known and used in popPK, for example as described in Mould D R et al., CPT Pharmacometrics Syst Pharmacol. 2013 April; 2 (4): e38; Guidance for Industry Population Pharmacokinetics, by U.S. Department of Health and Human Services Food and Drug Administration Center for Drug Evaluation and Research (CDER) Center for Biologics Evaluation and Research (CBER), February 1999. In some embodiments, the various popPK parameters in the dose termination methods, including the various rate parameters described in this Section (Section 4.6), can have the value as described in Section 5. In other embodiments, the various popPK parameters in the dose termination methods, including the various rate parameters described in this Section (Section 4.6), can be determined as described in Section 5.

“Population pharmacokinetic model” or “popPK model” is a model integrating the mathematical simulations of the absorption, distribution, metabolism and elimination of a drug and their metabolites to fit and/or predict the drug concentrations among a patient population, wherein such model can fit and/or predict the observed time course of drug concentrations among the patient population receiving clinically relevant doses of the drug and variability in the drug concentrations among such patient population. Such popPK model can predict the time course of drug concentrations among the patient populations receiving a given dose, and thus can simulate and determine the dose for an intended drug level in a patient population. In some embodiments, the popPK model comprises or consists of the popPK model described in Section 5.

“Whole-body physiologically based pharmacokinetic model” or “whole-body PBPK model” is a model integrating and mapping the absorption, distribution, metabolism and elimination of a drug and their metabolites onto a physiologically realistic compartmental structure, including body tissues, fluids, organs, and/or systems. Such whole-body PBPK model can have two distinctive set of parameters: (i) a drug independent subset, derived from the underlying physiological processes (e.g. diffusion and transport), which can be available as known and practiced in the field or determined specifically for a specific patient population as known and practiced in the field; and (ii) a drug-specific subset characterizing the pharmacokinetic properties of the particular drug and derived from clinical or preclinical studies. Such whole-body PBPK model can fit and/or predict the observed time course of drug concentrations in the patient receiving clinically relevant doses of the drug. Such whole-body PBPK model can predict the time course of drug concentrations in the patient receiving a given dose, and thus can simulate and determine the dose for an intended drug level in the patient. In some embodiments, the whole-body PBPK model comprises or consists of the whole-body PBPK model described in Section 5.

As is clear from the description, the dose determination method provided herein can be used to determine the effective dose, the induction regimen, and/or the maintenance regimen of the TL1A inhibitors for the various embodiments of the combination therapies. Therefore, the various embodiments described herein for the elements recited in the dose determination methods are also provided for the dose determination methods, including the various embodiments on the anti-TL1A antibodies or antigen binding fragments (e.g. in this Section (Section 4.6) and Sections 4.3.1(a) and 5), those on the effective dose (e.g. in this Section (Section 4.6) and Section 5), those on the induction regimen (e.g. in this Section (Section 4.6) and Section 5), those on the maintenance regimen (e.g. in this Section (Section 4.6) and Section 5), those on the diseased tissues, and/or those on the corresponding or reference tissues (e.g. in this Section (Section 4.6) and Section 5).

In some embodiments of the effective dose of TL1A inhibitors in the combination therapy provided herein, including in this Section (Section 4.6, e.g. each paragraph of Section 4.6), the concentration of TL1A is the concentration of free TL1A. In certain embodiments of the effective dose of TL1A inhibitors in the combination therapy provided herein, including in this Section (Section 4.6, e.g. each paragraph of Section 4.6), the concentration of TL1A in the diseased tissue referred to in the effective dose of TL1A inhibitors in the combination therapy is the concentration of free TL1A in the diseased tissue. In some embodiments of the effective dose of TL1A inhibitors in the combination therapy provided herein, including in this Section (Section 4.6, e.g. each paragraph of Section 4.6), the concentration of TL1A in a corresponding tissue or reference tissue is the concentration of free TL1A in the corresponding tissue or reference tissue. In certain other embodiments of the effective dose of TL1A inhibitors in the combination therapy provided herein, including in this Section (Section 4.6, e.g. each paragraph of Section 4.6), the concentration of TL1A in the diseased tissue referred to in the effective dose of TL1A inhibitors in the combination therapy is the concentration of free TL1A in the diseased tissue and the concentration of TL1A in a corresponding tissue or reference tissue is the concentration of free TL1A in the corresponding tissue or reference tissue. As used herein, free TL1A means TL1A not neutralized or bound by the TL1A inhibitor. Such free TL1A is the TL1A that can engage DR3 and trigger TL1A mediated signaling or functions.

In some embodiments, the term “therapeutically effective amount” refers to an amount of an inhibitor effective to “treat” a disease or disorder in a subject or mammal. In some cases, therapeutically effective amount of the drug reduces the severity of symptoms of the disease or disorder. In some instances, the disease or disorder comprises inflammatory bowel disease (IBD), Crohn's disease (CD), or ulcerative colitis (UC). In some instances, the IBD, CD, and/or UC are severe or medically refractory forms of the IBD, CD, and/or UC. Non-limiting examples of symptoms of IBD, CD, and/or UC include, but are not limited to, diarrhea, fever, fatigue, abdominal pain, abdominal cramping, inflammation, ulceration, nausea, vomiting, bleeding, blood in stool, reduced appetite, and weight loss.

In some embodiments, the terms, “treat” or “treating” as used herein refer to both therapeutic treatment and prophylactic or preventative measures (e.g., disease progression), wherein the object is to prevent or slow down (lessen) the targeted pathologic condition. Therapeutic treatment includes alleviating the condition and alleviating symptoms of the condition. In some aspects provided herein, subjects in need of treatment include those already with a disease or condition, as well as those susceptible to develop the disease or condition. The disease or condition may comprise an inflammatory disease or condition.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins P A, USA) (2000).

For the treatment of the disease, the appropriate dosage of a TL1A inhibitor or an IL23 inhibitor depends on the type of disease to be treated, the severity and course of the disease, the responsiveness of the disease, whether the inhibitor is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates. The TL1A inhibitor or the IL23 inhibitor can be administered one time or over a series of treatments lasting from several days to several months, or until a cure is effected or a diminution of the disease state is achieved (e.g., treatment or amelioration of IBD symptoms). The duration of treatment depends upon the subject's clinical progress and responsiveness to therapy. In certain embodiments, dosage is from 0.01 μg to 100 mg per kg of body weight, and can be given once or more daily, weekly, monthly or yearly.

In some embodiments of the effective dose of the TL1A inhibitors in the combination therapy, the subject is administered a dose of up to about 1000 mg of the TL1A inhibitors. In some embodiments, the subject is administered a dose from about 150 mg to about 1000 mg of the TL1A inhibitors. In some cases, the dose is about 150 mg to about 900 mg, about 150 mg to about 800 mg, about 150 mg to about 700 mg, about 150 mg to about 600 mg, about 150 mg to about 500 mg, about 150 mg to about 400 mg, about 150 mg to about 300 mg, about 150 mg to about 200 mg, about 160 mg to about 1000 mg, about 160 mg to about 900 mg, about 160 mg to about 800 mg, about 160 mg to about 700 mg, about 160 mg to about 600 mg, about 160 mg to about 500 mg, about 160 mg to about 400 mg, about 160 mg to about 300 mg, about 160 mg to about 200 mg, about 170 mg to about 1000 mg, about 170 mg to about 900 mg, about 170 mg to about 800 mg, about 170 mg to about 700 mg, about 170 mg to about 600 mg, about 170 mg to about 500 mg, about 170 mg to about 400 mg, about 170 mg to about 300 mg, about 170 mg to about 200 mg, about 175 mg to about 1000 mg, about 175 mg to about 900 mg, about 175 mg to about 800 mg, about 175 mg to about 700 mg, about 175 mg to about 600 mg, about 175 mg to about 500 mg, about 175 mg to about 400 mg, about 175 mg to about 300 mg, about 175 mg to about 200 mg, about 180 mg to about 1000 mg, about 180 mg to about 900 mg, about 180 mg to about 800 mg, about 180 mg to about 700 mg, about 180 mg to about 600 mg, about 180 mg to about 500 mg, about 180 mg to about 400 mg, about 180 mg to about 300 mg, about 180 mg to about 200 mg, about 190 mg to about 1000 mg, about 190 mg to about 900 mg, about 190 mg to about 800 mg, about 190 mg to about 700 mg, about 190 mg to about 600 mg, about 190 mg to about 500 mg, about 190 mg to about 400 mg, about 190 mg to about 300 mg, about 190 mg to about 200 mg, about 200 mg to about 1000 mg, about 200 mg to about 900 mg, about 200 mg to about 800 mg, about 200 mg to about 700 mg, about 200 mg to about 600 mg, about 200 mg to about 500 mg, about 200 mg to about 400 mg, or about 200 mg to about 300 mg of the TL1A inhibitors. In some cases, the dose is about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900, about 950 mg, or about 1000 mg of the TL1A inhibitors.

In some cases, the TL1A inhibitors are administered in a fixed dose, e.g., about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900, about 950 mg, or about 1000 mg. In some cases, a TL1A inhibitor is administered based on weight (kg) of the subject. For instance, the TL1A inhibitor is administered at a dose of about 0.15 mg/kg to about 20 mg/kg, or about 0.15 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10.0 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg.

In some embodiments, a dose of a TL1A inhibitor is administered subcutaneously. In some embodiments, a dose of a TL1A inhibitor is administered intravenously.

For subcutaneous injection, the dose may be administered in one or multiple injections. As a non-limiting example, a dose comprising about 800 mg of a TL1A inhibitor may be administered in about 2, 3, 4, or 5 injections. As a further example, the dose comprising about 800 mg of a TL1A inhibitor is administered in about 4 injections of about 200 mg/mL. In some embodiments, the dose may be administered in one injection. For example, a dose comprising about 175-300 mg TL1A inhibitor is administered in one injection of about 175-250 mg/mL. As another example, a dose comprising about 175-300 mg TL1A inhibitor is administered in one injection of about 175-200 mg/mL.

In some embodiments, a dose and/or injection of TL1A inhibitor is administered in a volume of less than about 3 mL, less than about 2.9 mL, less than about 2.8 mL, less than about 2.7 mL, less than about 2.6 mL, less than about 2.5 mL, less than about 2.4 mL, less than about 2.3 mL, less than about 2.2 mL, less than about 2.1 mL, less than about 2 mL, less than about 1.9 mL, less than about 1.8 mL, less than about 1.7 mL, less than about 1.6 mL, less than about 1.5 mL, less than about 1.4 mL, less than about 1.3 mL, less than about 1.2 mL, less than about 1.1 mL, less than about 1.0 mL, less than about 0.9 mL, less than about 0.8 mL, or less than about 0.7 mL. The volume may be at least about 0.5 mL. The volume may be about 0.5 mL to about 3 mL, about 0.5 mL to about 2.9 mL, about 0.5 mL to about 2.8 mL, about 0.5 mL to about 2.7 mL, about 0.5 mL to about 2.6 mL, about 0.5 mL to about 2.5 mL, about 0.5 mL to about 2.4 mL, about 0.5 mL to about 2.3 mL, about 0.5 mL to about 2.2 mL, about 0.5 mL to about 2.1 mL, about 0.5 mL to about 2 mL, 0.5 mL to about 1.9 mL, 0.5 mL to about 1.8 mL, 0.5 mL to about 1.7 mL, 0.5 mL to about 1.6 mL, about 0.5 mL to about 1.0 mL, about 0.5 mL to about 0.9 mL, about 0.5 mL to about 0.8 mL, about 0.6 mL to about 3 mL, about 0.6 mL to about 2.9 mL, about 0.6 mL to about 2.8 mL, about 0.6 mL to about 2.7 mL, about 0.6 mL to about 2.6 mL, about 0.6 mL to about 2.5 mL, about 0.6 mL to about 2.4 mL, about 0.6 mL to about 2.3 mL, about 0.6 mL to about 2.2 mL, about 0.6 mL to about 2.1 mL, about 0.6 mL to about 2.0 mL, about 0.6 mL to about 1.9 mL, about 0.6 mL to about 1.8 mL, about 0.6 mL to about 1.7 mL, about 0.6 mL to about 1.6 mL, about 0.6 mL to about 1.5 mL, about 0.6 mL to about 1.4 mL, about 0.6 mL to about 1.3 mL, about 0.6 mL to about 1.2 mL, about 0.6 mL to about 1.1 mL, about 0.6 mL to about 1.0 mL, about 0.6 mL to about 0.9 mL, about 0.6 mL to about 0.8 mL, about 0.7 mL to about 3 mL, about 0.7 mL to about 2.9 mL, about 0.7 mL to about 2.8 mL, about 0.7 mL to about 2.7 mL, about 0.7 mL to about 2.6 mL, about 0.7 mL to about 2.5 mL, about 0.7 mL to about 2.4 mL, about 0.7 mL to about 2.3 mL, about 0.7 mL to about 2.2 mL, about 0.7 mL to about 2.1 mL, about 0.7 mL to about 2.0 mL, about 0.7 mL to about 1.9 mL, about 0.7 mL to about 1.8 mL, about 0.7 mL to about 1.7 mL, about 0.7 mL to about 1.6 mL, about 0.7 mL to about 1.5 mL, about 0.7 mL to about 1.4 mL, about 0.7 mL to about 1.3 mL, about 0.7 mL to about 1.2 mL, about 0.7 mL to about 1.1 mL, about 0.7 mL to about 1.0 mL, about 0.7 mL to about 0.9 mL, or about 0.7 mL to about 0.8 mL. In some embodiments, the concentration of the TL1A inhibitor in each dose and/or injection is about or greater than about 155, 160, 165, 170, 175, 180, 185, 190, 195, 200, 205, 210, 215, 220, or 225 mg/mL of the TL1A inhibitor.

In some embodiments, the combination therapy comprises administering more than one dose of the TL1A inhibitor. Subsequent doses may have the same amount, less than, or greater than the amount of the TL1A inhibitor as the first dose. A subsequent dose may be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the previous dose. A subsequent dose may be administered about 1, 2, 3, or 4 weeks after the previous dose. The one or more doses may be about 1, 2, 3, 4, 5, 6, 7, 8, 9, 10, 11, 12, 13, 14, 15, 16, 17, 18, 19, or 20 doses. In a non-limiting example, the TL1A inhibitor is administered in about 6 doses, optionally every other week. In another non-limiting example, the TL1A inhibitor is administered in about 12 doses, optionally weekly. In some embodiments, the one or more doses of the TL1A inhibitor are administered during an induction period. The induction period may be about 6, 7, 8, 9, 10, 11, 12, 13, 14, or 15 weeks. As a non-limiting example, the induction period is about 12 weeks. After the induction period, the subject may be further treated, e.g., with additional doses of the TL1A inhibitor in a maintenance period. In some embodiments, the maintenance period comprises administering the TL1A inhibitor every 1, 2, 3, 4, 5, 6, or 7 days, or every 1, 2, 3, or 4 weeks. In an example embodiment, the maintenance period comprises administering the TL1A inhibitor every 2 or 4 weeks. In a non-limiting embodiment, the first dose is an i.v. dose, and one or more subsequent doses is a s.c. dose. In some embodiments, one or more doses are i.v. doses. In some embodiments, one or more doses are s.c. doses. In some embodiments, an induction period comprises i.v. administration. In some embodiments, a maintenance period comprises s.c. administration.

In some embodiments, the combination therapy comprises administering to the subject a first dose of the TL1A inhibitor. In some embodiments, the dose comprises about 250 mg to about 1000 mg of the TL1A inhibitor, about 400 mg to about 600 mg, about 700 mg to about 800 mg, or about 250 mg, about 300 mg, about 325 mg, about 350 mg, about 375 mg, about 400 mg, about 425 mg, about 450 mg, about 475 mg, about 500 mg, about 525 mg about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg or about 1000 mg TL1A inhibitor. In some embodiments, the first dose comprises about 800 mg TL1A inhibitor. In example embodiments, the first dose comprises about 800 mg TL1A inhibitor administered subcutaneously. In example embodiments, the first dose comprises about 500 mg TL1A inhibitor administered intravenously.

In some embodiments, the combination therapy comprises administering to a subject the first dose of the TL1A inhibitor at a first time point and a second dose of the TL1A inhibitor at a second time point. In some cases, the second time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the first time point. In some cases, the second time point is about 1, 2, 3, or 4 weeks after the first time point. In some cases, the second dose comprises the same amount of the TL1A inhibitor as the first dose. In some cases, the second dose comprises a different amount of the TL1A inhibitor as the first dose. In some cases, the second dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg about 550 mg, about 600 mg, about 650 mg, or about 700 mg TL1A inhibitor. In example embodiments, the second dose comprises about 175-300 mg TL1A inhibitor administered subcutaneously about 1 week after the first dose. In example embodiments, the second dose comprises about 500 mg TL1A inhibitor administered intravenously about 2 weeks after the first dose.

In some embodiments, the combination therapy comprises administering to the subject a third dose of the TL1A inhibitor at a third time point. In some cases, the third time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the second time point. In some cases, the third time point is about 1, 2, 3, or 4 weeks after the second time point. In some cases, the third dose comprises the same amount of the TL1A inhibitor as the second dose. In some cases, the third dose comprises a different amount of the TL1A inhibitor as the second dose. In some cases, the third dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the third dose comprises about 175-300 mg TL1A inhibitor administered subcutaneously about 1 week after the second dose. In example embodiments, the third dose comprises about 500 mg TL1A inhibitor administered intravenously about 2 weeks after the second dose.

In some embodiments, the combination therapy comprises administering to the subject a fourth dose of the TL1A inhibitor at a fourth time point. In some cases, the fourth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the third time point. In some cases, the fourth time point is about 1, 2, 3, or 4 weeks after the third time point. In some cases, the fourth dose comprises the same amount of the TL1A inhibitor as the third dose. In some cases, the fourth dose comprises a different amount of the TL1A inhibitor as the third dose. In some cases, the fourth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the fourth dose comprises about 175-300 mg TL1A inhibitor administered subcutaneously about 1 week after the third dose. In example embodiments, the fourth dose comprises about 500 mg TL1A inhibitor administered intravenously about 2 weeks after the third dose.

In some embodiments, the combination therapy comprises administering to the subject a fifth dose of the TL1A inhibitor at a fifth time point. In some cases, the fifth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the fourth time point. In some cases, the fifth time point is about 1, 2, 3, or 4 weeks after the fourth time point. In some cases, the fifth dose comprises the same amount of the TL1A inhibitor as the fourth dose. In some cases, the fifth dose comprises a different amount of the TL1A inhibitor as the fourth dose. In some cases, the fifth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the fifth dose comprises about 175-300 mg TL1A inhibitor administered subcutaneously about 1 week after the fourth dose. In example embodiments, the fifth dose comprises about 500 mg TL1A inhibitor administered intravenously about 2 weeks after the fourth dose.

In some embodiments, the combination therapy comprises administering to the subject a sixth dose of the TL1A inhibitor at a sixth time point. In some cases, the sixth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the fifth time point. In some cases, the sixth time point is about 1, 2, 3, or 4 weeks after the fifth time point. In some cases, the sixth dose comprises the same amount of the TL1A inhibitor as the fifth dose. In some cases, the sixth dose comprises a different amount of the TL1A inhibitor as the fifth dose. In some cases, the sixth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg about 500 mg, about 550 mg, about 600 mg, about 650 mg, or about 700 mg TL1A inhibitor. In example embodiments, the sixth dose comprises about 175-300 mg TL1A inhibitor administered subcutaneously about 1 week after the fifth dose. In example embodiments, the sixth dose comprises about 500 mg TL1A inhibitor administered intravenously about 2 weeks after the fifth dose.

In some embodiments, the combination therapy comprises administering to the subject a seventh dose of the TL1A inhibitor at a seventh time point. In some cases, the seventh time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the sixth time point. In some cases, the seventh time point is about 1, 2, 3, or 4 weeks after the sixth time point. In some cases, the seventh dose comprises the same amount of the TL1A inhibitor as the sixth dose. In some cases, the seventh dose comprises a different amount of the TL1A inhibitor as the sixth dose. In some cases, the seventh dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the seventh dose comprises about 175-300 mg TL1A inhibitor administered subcutaneously about 1 week after the sixth dose. In example embodiments, the seventh dose comprises about 500 mg TL1A inhibitor administered intravenously about 2 weeks after the sixth dose.

In some embodiments, the combination therapy comprises administering to the subject an eighth dose of the TL1A inhibitor at an eighth time point. In some cases, the eighth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the seventh time point. In some cases, the eighth time point is about 1, 2, 3, or 4 weeks after the seventh time point. In some cases, the eighth dose comprises the same amount of the TL1A inhibitor as the seventh dose. In some cases, the eighth dose comprises a different amount of the TL1A inhibitor as the seventh dose. In some cases, the eighth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the eighth dose comprises about 175-300 mg TL1A inhibitor administered subcutaneously about 1 week after the seventh dose. In example embodiments, the eighth dose comprises about 500 mg TL1A inhibitor administered intravenously about 2 weeks after the seventh dose.

In some embodiments, the combination therapy comprises administering to the subject a ninth dose of the TL1A inhibitor at a ninth time point. In some cases, the ninth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the eighth time point. In some cases, the ninth time point is about 1, 2, 3, or 4 weeks after the eighth time point. In some cases, the ninth dose comprises the same amount of the TL1A inhibitor as the eighth dose. In some cases, the ninth dose comprises a different amount of the TL1A inhibitor as the eighth dose. In some cases, the ninth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the ninth dose comprises about 175-300 mg TL1A inhibitor administered subcutaneously about 1 week after the eighth dose. In example embodiments, the ninth dose comprises about 500 mg TL1A inhibitor administered intravenously about 2 weeks after the eighth dose.

In some embodiments, the combination therapy comprises administering to the subject a tenth dose of the TL1A inhibitor at a tenth time point. In some cases, the tenth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the ninth time point. In some cases, the tenth time point is about 1, 2, 3, or 4 weeks after the ninth time point. In some cases, the tenth dose comprises the same amount of the TL1A inhibitor as the ninth dose. In some cases, the tenth dose comprises a different amount of the TL1A inhibitor as the ninth dose. In some cases, the tenth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the tenth dose comprises about 175-300 mg TL1A inhibitor administered subcutaneously about 1 week after the ninth dose. In example embodiments, the tenth dose comprises about 500 mg TL1A inhibitor administered intravenously about 2 weeks after the ninth dose.

In some embodiments, the combination therapy comprises administering to the subject an eleventh dose of the TL1A inhibitor at an eleventh time point. In some cases, the eleventh time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the tenth time point. In some cases, the eleventh time point is about 1, 2, 3, or 4 weeks after the tenth time point. In some cases, the eleventh dose comprises the same amount of the TL1A inhibitor as the tenth dose. In some cases, the eleventh dose comprises a different amount of the TL1A inhibitor as the tenth dose. In some cases, the eleventh dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the eleventh dose comprises about 175-300 mg TL1A inhibitor administered subcutaneously about 1 week after the tenth dose. In example embodiments, the eleventh dose comprises about 500 mg TL1A inhibitor administered intravenously about 2 weeks after the tenth dose.

In some embodiments, the combination therapy comprises administering to the subject a twelfth dose of the TL1A inhibitor at a twelfth time point. In some cases, the twelfth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the eleventh time point. In some cases, the twelfth time point is about 1, 2, 3, or 4 weeks after the eleventh time point. In some cases, the twelfth dose comprises the same amount of the TL1A inhibitor as the eleventh dose. In some cases, the twelfth dose comprises a different amount of the TL1A inhibitor as the eleventh dose. In some cases, the twelfth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the twelfth dose comprises about 175-300 mg TL1A inhibitor administered subcutaneously about 1 week after the eleventh dose. In example embodiments, the twelfth dose comprises about 500 mg TL1A inhibitor administered intravenously about 2 weeks after the eleventh dose.

In some embodiments, the combination therapy comprises administering to the subject a thirteenth dose of the TL1A inhibitor at a thirteenth time point. In some cases, the thirteenth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the twelfth time point. In some cases, the thirteenth time point is about 1, 2, 3, or 4 weeks after the twelfth time point. In some cases, the thirteenth dose comprises the same amount of the TL1A inhibitor as the twelfth dose. In some cases, the thirteenth dose comprises a different amount of the TL1A inhibitor as the twelfth dose. In some cases, the thirteenth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the thirteenth dose comprises about 175-300 mg TL1A inhibitor administered subcutaneously about 1 week after the twelfth dose. In example embodiments, the thirteenth dose comprises about 500 mg TL1A inhibitor administered intravenously about 2 weeks after the twelfth dose.

In some embodiments, the combination therapy comprises administering to the subject a fourteenth dose of the TL1A inhibitor at a fourteenth time point. In some cases, the fourteenth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the thirteenth time point. In some cases, the fourteenth time point is about 1, 2, 3, or 4 weeks after the thirteenth time point. In some cases, the fourteenth dose comprises the same amount of the TL1A inhibitor as the thirteenth dose. In some cases, the fourteenth dose comprises a different amount of the TL1A inhibitor as the thirteenth dose. In some cases, the fourteenth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the fourteenth dose comprises about 175-300 mg TL1A inhibitor administered subcutaneously about 1 week after the thirteenth dose. In example embodiments, the fourteenth dose comprises about 500 mg TL1A inhibitor administered intravenously about 2 weeks after the thirteenth dose.

In some embodiments, the combination therapy comprises administering to the subject a fifteenth dose of the TL1A inhibitor at a fifteenth time point. In some cases, the fifteenth time point is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the fourteenth time point. In some cases, the fifteenth time point is about 1, 2, 3, or 4 weeks after the fourteenth time point. In some cases, the fifteenth dose comprises the same amount of the TL1A inhibitor as the fourteenth dose. In some cases, the fifteenth dose comprises a different amount of the TL1A inhibitor as the fourteenth dose. In some cases, the fifteenth dose comprises about 150 mg to about 700 mg, about 150 mg to about 300 mg, about 150 mg to about 225 mg, about 175 mg to about 225 mg, about 400 mg to about 600 mg, about 450 mg to about 550 mg, about 475 mg to about 525 mg, or about 150 mg, about 160 mg, about 170 mg, about 180 mg, about 190 mg, about 200 mg, about 210 mg, about 220, about 230 mg, about 240 mg, about 250 mg, about 300 mg, about 350 mg, about 400 mg, about 450 mg, about 500 mg, about 550 mg, about 600 mg, about 650 mg, or about 700 mg TL1A inhibitor. In example embodiments, the fifteenth dose comprises about 175-300 mg TL1A inhibitor administered subcutaneously about 1 week after the fourteenth dose. In example embodiments, the fifteenth dose comprises about 500 mg TL1A inhibitor administered intravenously about 2 weeks after the fourteenth dose.

In some embodiments where the subject is responsive to treatment, the subject is further treated with the TL1A inhibitor in a maintenance phase. As a non-limiting example, treatment comprises 1 to about 20 doses, 1 to about 12 doses, 1 to about 6 doses, about 6 doses or about 12 doses. In some embodiments, the maintenance phase comprises administration of about 150 mg to about 250 mg, about 150 mg to about 225 mg, about 150 mg to about 200 mg, about 175 mg to about 225 mg, about 175 to about 200 mg, about 150 mg, about 160 mg, about 170 mg, about 180 mg, about 190 mg, about 200 mg, about 210 mg about 220, about 230 mg, about 240 mg, or about 250 mg TL1A inhibitor in one or more doses. In some cases, maintenance comprises administration of a dose of the TL1A inhibitor every 1, 2, 3, or 4 weeks. In some cases, maintenance comprises administration of a dose of about 175 mg to about 300 mg every 2 weeks. In some cases, maintenance comprises administration of a dose of about 175 mg to about 300 mg every 4 weeks. In some cases, the administration is subcutaneous. In some cases, the administration is intravenous.

In one aspect, a combination therapy of treatment comprises administrating a TL1A inhibitor to the subject a first dose on day 0, a second dose on day 7, a third dose on day 14, a fourth dose on day 21, a fifth dose on day 28, a sixth dose on day 35, a seventh dose on day 42, an eighth dose on day 49, a ninth dose on day 56, a tenth dose on day 63, an eleventh dose on day 70, a twelfth dose on day 77, and optionally a thirteenth dose is administered on day 84. In some embodiments, the first dose comprises about 500-1000 mg or about 800 mg TL1A inhibitor. In some embodiments, the second dose comprises about 175-300 mg TL1A inhibitor. In some embodiments, the third dose comprises about 175-300 mg TL1A inhibitor. In some embodiments, the fourth dose comprises about 175-300 mg TL1A inhibitor. In some embodiments, the fifth dose comprises about 175-300 mg TL1A inhibitor. In some embodiments, the sixth dose comprises about 175-300 mg TL1A inhibitor. In some embodiments, the seventh dose comprises about 175-300 mg TL1A inhibitor. In some embodiments, the eighth dose comprises about 175-300 mg TL1A inhibitor. In some embodiments, the ninth dose comprises about 175-300 mg TL1A inhibitor. In some embodiments, the tenth dose comprises about 175-300 mg TL1A inhibitor. In some embodiments, the eleventh dose comprises about 175-300 mg TL1A inhibitor. In some embodiments, the twelfth dose comprises about 175-300 mg TL1A inhibitor. In some embodiments, the thirteenth dose comprises about 175-300 mg TL1A inhibitor. The TL1A inhibitor may be administered subcutaneously, e.g., in a composition disclosed herein. In some embodiments where the subject is responsive to treatment, the subject is further treated with the TL1A inhibitor in a maintenance phase. In some cases, maintenance comprises administration of a dose of about 175 mg to about 300 mg every 2 weeks. In some cases, maintenance comprises administration of a dose of about 175 mg to about 300 mg every 4 weeks. In some cases, the maintenance administration is subcutaneous. In some cases, the maintenance administration is intravenous. In a non-limiting embodiment, the first dose is an i.v. dose, and one or more subsequent doses is a s.c. dose. For instance, in some cases, the induction period comprises i.v. administration and the maintenance period comprises s.c. administration.

In one aspect, a combination therapy of treatment comprises administrating a TL1A inhibitor to the subject a first dose on day 0, a second dose on day 14, a third dose on day 28, a fourth dose on day 42, a fifth dose on day 56, a sixth dose on day 70, and optionally a seventh dose on day 84. In some embodiments, the first dose comprises about 400-600 mg or about 500 mg TL1A inhibitor. In some embodiments, the second dose comprises about 400-600 mg TL1A inhibitor. In some embodiments, the third dose comprises about 400-600 mg TL1A inhibitor. In some embodiments, the fourth dose comprises about 400-600 mg TL1A inhibitor. In some embodiments, the fifth dose comprises about 400-600 mg TL1A inhibitor. In some embodiments, the sixth dose comprises about 400-600 mg TL1A inhibitor. In some embodiments, the seventh dose comprises about 400-600 mg TL1A inhibitor. The TL1A inhibitor may be administered intravenously, e.g., by diluting a composition herein to a suitable volume for administration, such as about 250 mL. In some cases, maintenance comprises administration of a dose of about 175 mg to about 300 mg every 2 weeks. In some cases, maintenance comprises administration of a dose of about 175 mg to about 300 mg every 4 weeks. In some cases, the maintenance administration is subcutaneous. In some cases, the maintenance administration is intravenous. In a non-limiting embodiment, the first dose is an i.v. dose, and one or more subsequent doses is a s.c. dose. For instance, in some cases, the induction period comprises i.v. administration and the maintenance period comprises s.c. administration.

### 6.7 Methods of Treatment with the Combination of a TL1A Inhibitor and an IL23 Inhibitor and the Compositions Therefor

The role of IL-23 in IBD has been clinically validated with both p40- and p19-specific systemic monoclonal antibodies that are efficacious and safe in both CD and UC patients, including those that previously failed anti-TNFα therapy (Sands, B. E. et al. Gastroenterology 153, 77-86.e6 (2017); Feagan, B. G. et al. Lancet 389, 1699-1709 (2017); Sandborn, W. J. et al. Gastroenterology 158, 537-549.e10 (2020); Feagan, B. G. et al. Lancet Gastroenterol. Hepatol. 3, 671-680 (2018); Sands, B. E. et al. N. Engl. J. Med. 381, 1201-1214 (2019); Hanžel, J. & D'Haens, G. R. Expert Opin. Biol. Ther. 20, 399-406 (2020), the disclosures of all of which are hereby incorporate by reference in their entirety). Multiple lines of evidence support a role for the IL-23/IL-23R axis in IBD. Without being bound by the theory, the disclosure provides that IL-23 and IL-23R expression are increased in inflamed IBD mucosal tissue and IL-23 production by macrophages and dendritic cells promotes the expression of further proinflammatory cytokines by mucosal T cells and innate lymphoid cells in the IBD gut (Monteleone, G., Monteleone, I. & Pallone, F. Med. Inflamm. 2009, 1-7 (2009); Liu, Z. et al. J. Leukoc. Biol. 89, 597-606 (2011); Kamada, N. et al. J. Clin. Investig. 118, 2269-2280 (2008); Geremia, A. et al. J. Exp. Med. 208, 1127-1133 (2011); the disclosures of all of which are hereby incorporate by reference in their entirety). Furthermore, the upregulation of mucosal IL-23p19 and IL-23R expression, and the expansion of apoptosis-resistant intestinal TNFR2+IL-23R+ T cells, are associated with resistance to anti-TNFα therapy in CD patients (Schmitt, H. et al. Gut. 68, 814-828 (2019), the disclosures of which are hereby incorporate by reference in their entirety). Additionally, IL23 is one of the key promotors of the T helper 17 (Th17) cell pathway which has been implicated in many inflammatory diseases and conditions However, there is an unmet need for new therapeutics with sustained efficacy across a broader patient population. The present disclosure thus provides that reduction of IL23 mediated activity is not sufficient for disease reduction in all patients. Thus, targeting two immune mechanisms, e.g. (i) blocking IL-23-mediated proinflammatory cytokines production by resident gut immune cells and IL-23 dependent Th17 expansion and maintenance and with an IL23 inhibitor and (ii) modulation of resident tissue immune cells by neutralizing TL1A, can act in a synergistic manner.

Accordingly, the disclosure provides that the combination of a TL1A inhibitor and an IL23 inhibitor can be used in a method to treat an inflammatory disease or condition in a subject by administering the combination of the TL1A inhibitor and the IL23 inhibitor thereof described herein to the subject. More specifically, the combination of a TL1A inhibitor and an IL23 inhibitor thereof provided herein can be used in a method to treat an inflammatory bowel disease (“IBD”) in a subject by administering the combination of a TL1A inhibitor and an IL23 inhibitor described herein to the subject. In various embodiments, IBD is Crohn's Disease (CD) and/or ulcerative colitis (UC).

Provided herein are methods of treating an inflammatory disease or condition in a subject by administering a TL1A inhibitor and an IL23 inhibitor, each as described herein, to the subject. In example embodiments, the inflammatory disease or condition is inflammatory bowel disease. In various embodiments, IBD is CD and/or UC. In some embodiments, the IBD patient has with fibrosis. In some embodiments, the IBD is a severe form of IBD. In some embodiments, the IBD is a moderate to severe form of IBD. In some embodiments, the IBD is a moderate form of IBD. In various other embodiments, the subject is determined to have an increased TL1A expression. In some embodiments, the administration of a therapeutically effective amount of a TL1A inhibitor causes a decrease in TL1A in the subject treated. In example embodiments, the TL1A inhibitor comprises any one of the anti-TL1A antibody embodiments provided herein. In some embodiments, the anti-TL1A antibody comprises antibody A, B, C, D, E, F, G, H, I, A2, B2, C2, D2, E2, F2, G2, H2, or 12. In some embodiments, the anti-TL1A antibody comprises any one of the antibodies of Table 1. As a non-limiting example, the anti-TL1A antibody comprises antibody A219.

In one aspect, provided herein is a method of treating an inflammatory disease or condition in a subject comprising administering to the subject a first composition comprising a first therapeutically effective amount of an inhibitor of tumor necrosis factor-like protein 1A (“TL1A” and such inhibitor, “TL1A inhibitor”) and administering to the subject a second composition comprising a second therapeutically effective amount of an inhibitor of interleukin 23 (“IL23 inhibitor”).

In another aspect, provided herein is method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor.

In another aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor.

In yet another aspect, provided herein is a method of treating an inflammatory disease or condition in a subject comprising administering to the subject a composition comprising a first therapeutically effective amount of a TL1A inhibitor and a second therapeutically effective amount of an IL23 inhibitor.

In one aspect, provided herein is a method of treating an inflammatory disease or condition in a subject comprising administering to the subject a first composition comprising a first therapeutically effective amount of a TL1A inhibitor and administering to the subject a second composition comprising a second therapeutically effective amount of an IL23 inhibitor, wherein the TL1A inhibitor comprises any TL1A inhibitor provided in Section 4.3.1. In one aspect, provided herein is a method of treating an inflammatory disease or condition in a subject comprising administering to the subject a first composition comprising a first therapeutically effective amount of a TL1A inhibitor and administering to the subject a second composition comprising a second therapeutically effective amount of an IL23 inhibitor, wherein the IL23 inhibitor comprises any IL23 inhibitor provided in Section 4.3.2. In one aspect, provided herein is a method of treating an inflammatory disease or condition in a subject comprising administering to the subject a first composition comprising a first therapeutically effective amount of a TL1A inhibitor and administering to the subject a second composition comprising a second therapeutically effective amount of an IL23 inhibitor, wherein the TL1A inhibitor comprises any TL1A inhibitor provided in Section 4.3.1 and wherein the IL23 inhibitor comprises any IL23 inhibitor provided in Section 4.3.2.

In another aspect, provided herein is method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1. In another aspect, provided herein is method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor, wherein the IL23 inhibitor comprises any IL23 inhibitor provided in Section 4.3.2. In another aspect, provided herein is method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and wherein the IL23 inhibitor comprises any IL23 inhibitor provided in Section 4.3.2.

In another aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1. In another aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor, wherein the IL23 inhibitor comprises any IL23 inhibitor provided in Section 4.3.2. In another aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and wherein the IL23 inhibitor comprises any IL23 inhibitor provided in Section 4.3.2.

In yet another aspect, provided herein is a method of treating an inflammatory disease or condition in a subject comprising administering to the subject a composition comprising a first therapeutically effective amount of a TL1A inhibitor and a second therapeutically effective amount of an IL23 inhibitor, wherein the TL1A inhibitor comprises any TL1A inhibitor provided in Section 4.3.1. In yet another aspect, provided herein is a method of treating an inflammatory disease or condition in a subject comprising administering to the subject a composition comprising a first therapeutically effective amount of a TL1A inhibitor and a second therapeutically effective amount of an IL23 inhibitor, wherein the IL23 inhibitor comprises any IL23 inhibitor provided in Section 4.3.2. In yet another aspect, provided herein is a method of treating an inflammatory disease or condition in a subject comprising administering to the subject a composition comprising a first therapeutically effective amount of a TL1A inhibitor and a second therapeutically effective amount of an IL23 inhibitor, wherein the TL1A inhibitor comprises any TL1A inhibitor provided in Section 4.3.1 and wherein the IL23 inhibitor comprises any IL23 inhibitor provided in Section 4.3.2.

In one aspect, provided herein is a method of treating an inflammatory disease or condition in a subject comprising administering to the subject a first composition comprising a first therapeutically effective amount of a TL1A inhibitor and administering to the subject a second composition comprising a second therapeutically effective amount of an IL23 inhibitor, wherein: (1) the TL1A inhibitor comprises any TL1A inhibitor provided in Section 4.3.1 and/or the IL23 inhibitor comprises any IL23 inhibitor provided in Section 4.3.2, and (2) the first therapeutically effective amount comprises any amount provided for TL1A inhibitor in Sections 4.5, 4.6 and 4.7. In one aspect, provided herein is a method of treating an inflammatory disease or condition in a subject comprising administering to the subject a first composition comprising a first therapeutically effective amount of a TL1A inhibitor and administering to the subject a second composition comprising a second therapeutically effective amount of an IL23 inhibitor, wherein: (1) the TL1A inhibitor comprises any TL1A inhibitor provided in Section 4.3.1 and/or the IL23 inhibitor comprises any IL23 inhibitor provided in Section 4.3.2, and (2) the second therapeutically effective amount comprises any amount provided for IL23 inhibitor in Sections 4.3.2, 4.5, and 4.7. In one aspect, provided herein is a method of treating an inflammatory disease or condition in a subject comprising administering to the subject a first composition comprising a first therapeutically effective amount of a TL1A inhibitor and administering to the subject a second composition comprising a second therapeutically effective amount of an IL23 inhibitor, wherein: (1) the TL1A inhibitor comprises any TL1A inhibitor provided in Section 4.3.1 and/or the IL23 inhibitor comprises any IL23 inhibitor provided in Section 4.3.2, and (2) the first therapeutically effective amount comprises any amount provided for TL1A inhibitor in Sections 4.5, 4.6 and 4.7 and the second therapeutically effective amount comprises any amount provided for IL23 inhibitor in Sections 4.3.2, 4.5, and 4.7.

In another aspect, provided herein is method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the first therapeutically effective amount comprises any amount provided for TL1A inhibitor in Sections 4.5, 4.6 and 4.7. In another aspect, provided herein is method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the second therapeutically effective amount comprises any amount provided for IL23 inhibitor in Sections 4.3.2, 4.5, and 4.7. In another aspect, provided herein is method of treating an inflammatory disease or condition in a subject comprising: (a) administering an induction regimen to the subject comprising (i) administering a first composition comprising a first therapeutically effective amount of a TL1A inhibitor and (ii) administering a second composition comprising a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the first therapeutically effective amount comprises any amount provided for TL1A inhibitor in Sections 4.5, 4.6 and 4.7 and the second therapeutically effective amount comprises any amount provided for IL23 inhibitor in Sections 4.3.2, 4.5, and 4.7.

In another aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the first therapeutically effective amount comprises any amount provided for TL1A inhibitor in Sections 4.5, 4.6 and 4.7. In another aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the second therapeutically effective amount comprises any amount provided for IL23 inhibitor in Sections 4.3.2, 4.5, and 4.7. In another aspect, provided herein is a method of treating an inflammatory disease or condition in a subject comprising: (a) administering to the subject an induction regimen, wherein the induction regimen comprises a composition comprising a first therapeutically effective amount of a TL1A inhibitor and a second therapeutically effective amount of an IL23 inhibitor; and (b) administering a maintenance regimen to the subject after the induction regimen, wherein the maintenance regimen comprises the TL1A inhibitor or the IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the first therapeutically effective amount comprises any amount provided for TL1A inhibitor in Sections 4.5, 4.6 and 4.7 and the second therapeutically effective amount comprises any amount provided for IL23 inhibitor in Sections 4.3.2, 4.5, and 4.7.

In yet another aspect, provided herein is a method of treating an inflammatory disease or condition in a subject comprising administering to the subject a composition comprising a first therapeutically effective amount of a TL1A inhibitor and a second therapeutically effective amount of an IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the first therapeutically effective amount comprises any amount provided for TL1A inhibitor in Sections 4.5, 4.6 and 4.7. In yet another aspect, provided herein is a method of treating an inflammatory disease or condition in a subject comprising administering to the subject a composition comprising a first therapeutically effective amount of a TL1A inhibitor and a second therapeutically effective amount of an IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the second therapeutically effective amount comprises any amount provided for IL23 inhibitor in Sections 4.3.2, 4.5, and 4.7. In yet another aspect, provided herein is a method of treating an inflammatory disease or condition in a subject comprising administering to the subject a composition comprising a first therapeutically effective amount of a TL1A inhibitor and a second therapeutically effective amount of an IL23 inhibitor, wherein the TL1A inhibitor comprises any TL1A inhibitor provided in Section 4.3.1 and/or the IL23 inhibitor comprises any IL23 inhibitor provided in Section 4.3.2, and wherein the first therapeutically effective amount comprises any amount provided for TL1A inhibitor in Sections 4.5, 4.6 and 4.7 and the second therapeutically effective amount comprises any amount provided for IL23 inhibitor in Sections 4.3.2, 4.5, and 4.7.

The therapeutically effective amounts for TL1A inhibitors have been provided above and below. Accordingly, in various embodiments of the methods of treating an inflammatory disease or condition provided herein, including in this Section (Section 4.7, e.g the preceding paragraphs), the maintenance regimen comprises a third therapeutically effective amount of the TL1A inhibitor. In various embodiments of the methods of treating an inflammatory disease or condition provided herein, including in this Section (Section 4.7), the maintenance regimen comprises a third therapeutically effective amount of the TL1A inhibitor, wherein the third therapeutically effective amount comprises any amount provided for TL1A inhibitor in Sections 4.5, 4.6 and 4.7.

Similarly, the therapeutically effective amounts for IL23 inhibitors have been provided above and below. Accordingly, in various embodiments of the methods of treating an inflammatory disease or condition provided herein, including in this Section (Section 4.7), the maintenance regimen comprises a fourth therapeutically effective amount of the IL23 inhibitor. In various embodiments of the methods of treating an inflammatory disease or condition provided herein, including in this Section (Section 4.7), the maintenance regimen comprises a fourth therapeutically effective amount of the IL23 inhibitor, wherein the fourth therapeutically effective amount comprises any amount provided for IL23 inhibitor in Sections 4.3.2, 4.5, and 4.7.

Additionally, in various embodiments of the methods of treating an inflammatory disease or condition provided herein, including in this Section (Section 4.7), the first therapeutically effective amount comprises any amount provided for TL1A inhibitor in Sections 4.5, 4.6 and 4.7. In some embodiments, the third therapeutically effective amount comprises any amount provided for TL1A inhibitor in Sections 4.5, 4.6 and 4.7. In some embodiments, the first therapeutically effective amount comprises any amount provided for TL1A inhibitor in Sections 4.5, 4.6 and 4.7 and the third therapeutically effective amount comprises any amount provided for TL1A inhibitor in Sections 4.5, 4.6 and 4.7.

In various embodiments of the methods of treating an inflammatory disease or condition provided herein, including in this Section (Section 4.7), the second therapeutically effective amount comprises any amount provided for IL23 inhibitor in Sections 4.3.2, 4.5, and 4.7. In some embodiments, the fourth therapeutically effective amount comprises any amount provided for IL23 inhibitor in Sections 4.3.2, 4.5, and 4.7. In some embodiments, the second therapeutically effective amount comprises any amount provided for IL23 inhibitor in Sections 4.3.2, 4.5, and 4.7 and the fourth therapeutically effective amount comprises any amount provided for IL23 inhibitor in Sections 4.3.2, 4.5, and 4.7.

In various embodiments of the methods of treating an inflammatory disease or condition provided herein, including in this Section (Section 4.7), the first therapeutically effective amount comprises any amount provided for TL1A inhibitor in Sections 4.5, 4.6 and 4.7, the third therapeutically effective amount comprises any amount provided for TL1A inhibitor in Sections 4.5, 4.6 and 4.7, the second therapeutically effective amount comprises any amount provided for IL23 inhibitor in Sections 4.3.2, 4.5, and 4.7, and/or the fourth therapeutically effective amount comprises any amount provided for IL23 inhibitor in Sections 4.3.2, 4.5, and 4.7.

The TL1A inhibitors and the IL23 inhibitors can be used in various ratios in the various methods of treating an inflammatory disease or condition provided herein, including in this Section (Section 4.7). In one embodiment, the molar ratio of the first therapeutically effective amount of the TL1A inhibitor to the second therapeutically effective amount of the IL23 inhibitor is about 100:1, about 95:1, about 90:1, about 85:1, about 80:1, about 75:1, about 70:1, about 65:1, about 60:1, about 55:1, about 50:1, about 45:1, about 40:1, about 35:1, about 30:1, about 25:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, or about 1:100.

Various inflammatory disease or conditions can be treated with the methods using a combination of a TL1A inhibitor and an IL23 inhibitor provided herein or treated with various compositions provided herein. As such, in various embodiments of the methods of treating an inflammatory disease or condition provided herein, including in this Section (Section 4.7) and Sections 2, 4.3, 4.5, and 4.6, the inflammatory disease or condition is inflammatory bowel disease (IBD). In some embodiments, the inflammatory disease or condition is ulcerative colitis (UC). In some embodiments, the inflammatory disease or condition is indeterminate colitis. In some embodiments, the inflammatory disease or condition is moderately active UC. In some embodiments, the inflammatory disease or condition is severely active UC. In some embodiments, the inflammatory disease or condition is Crohn's Disease (CD).

The characteristics and/or properties of the TL1A inhibitors have been described in Section 4.3.1 and provided above for the methods of treating an inflammatory disease or condition of this Section (Section 4.7). For example, in some embodiments, the TL1A inhibitor is an inhibitor of TL1A expression or an inhibitor of TL1A activity. In some other embodiments, the TL1A inhibitor is an anti-TL1A antibody or antigen binding fragment thereof. In one embodiment, the TL1A inhibitor binds to both monomeric TL1A and trimeric TL1A. In other embodiments, the TL1A inhibitor comprises an anti-TL1A antibody or antigen binding fragment thereof and the antibody or antigen binding fragment binds to both monomeric TL1A and trimeric TL1A. In one embodiment, the TL1A inhibitor comprises an anti-TL1A antibody or antigen binding fragment thereof, the antibody or antigen binding fragment binds to both monomeric TL1A and trimeric TL1A, and the TL1A inhibitor blocks interaction of TL1A to Death Receptor 3 (“DR3”). In one embodiment, the TL1A inhibitor blocks interaction of TL1A to Death Receptor 3 (“DR3”). In one embodiment, the TL1A inhibitor comprises an anti-TL1A antibody or antigen binding fragment thereof and the TL1A inhibitor blocks interaction of TL1A to Death Receptor 3 (“DR3”). In another embodiment, the TL1A inhibitor the TL1A inhibitor comprises an anti-TL1A antibody or antigen binding fragment thereof and binding affinity of the antibody or antigen binding fragment to monomeric TL1A as measured by dissociation equilibrium constant (K_{D-monomer}) is comparable to binding affinity of the antibody or antigen binding fragment to trimeric TL1A as measured by dissociation equilibrium constant (K_{D-trimer}). In yet another embodiment, the K_{D-monomer} is within 1.5, 2, 3, 4, 5, 6, 7, 8, 9, or 10 fold of the K_{D-trimer}. In a further embodiment, the K_{D-monomer} is no more than 0.06 nM. In one embodiment, the K_{D-trimer} is no more than 0.06 nM. In yet another embodiment, the K_{D-monomer} is no more than 0.06 nM and the K_{D-trimer} is no more than 0.06 nM.

Other functional characteristics and/or properties of the TL1A inhibitors have been described in Section 4.3.1 and provided above for the methods of treating an inflammatory disease or condition of this Section (Section 4.7). Accordingly, in some embodiments, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the monomeric TL1A in the blood of the subject is occupied by the anti-TL1A antibody or antigen binding fragment after administering the first therapeutically effective amount in the combination therapy with an anti-TL1A antibody and an IL23 inhibitor. In some other embodiments, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the trimeric TL1A in the blood of the subject is occupied by the anti-TL1A antibody or antigen binding fragment after administering the first therapeutically effective amount in the combination therapy with an anti-TL1A antibody and an IL23 inhibitor. In some embodiments, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the monomeric TL1A in the blood of the subject is occupied by the anti-TL1A antibody or antigen binding fragment after administering the first therapeutically effective amount in the combination therapy with an anti-TL1A antibody and an IL23 inhibitor. In some other embodiments, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the trimeric TL1A in the blood of the subject is occupied by the anti-TL1A antibody or antigen binding fragment after administering the first therapeutically effective amount in the combination therapy with an anti-TL1A antibody and an IL23 inhibitor.

Similarly, the effective dose for the TL1A inhibitors have been described in Sections 4.5, 4.6 and 4.7 and provided above for the methods of treating an inflammatory disease or condition of this Section (Section 4.7). For example, in some embodiments, the first therapeutically effective amount for the TL1A inhibitor in the combination therapy comprises 200 mg/dose, 250 mg/dose, 300 mg/dose, 350 mg/dose, 400 mg/dose, 450 mg/dose, 500 mg/dose, 550 mg/dose, 600 mg/dose, 650 mg/dose, 700 mg/dose, 750 mg/dose, 800 mg/dose, 850 mg/dose, 900 mg/dose, 950 mg/dose, 1000 mg/dose, 1100 mg/dose, 1200 mg/dose, 1250 mg/dose, 1300 mg/dose, 1400 mg/dose, 1500 mg/dose, 1600 mg/dose, 1700 mg/dose, 1750 mg/dose, 1800 mg/dose, 1900 mg/dose, or 2000 mg/dose TL1A inhibitor. In one embodiment, the first therapeutically effective amount for the TL1A inhibitor in the combination therapy comprises 1, 2, 3, 4, 5, 6, 7, 8, 9, 10, 11, 12, or more doses. In another embodiment, the first therapeutically effective amount for the TL1A inhibitor in the combination therapy comprises (i) 1000 mg/dose on week 0, 1000 mg/dose on week 2, 1000 mg/dose on week 6, and 1000 mg/dose on week 10; (ii) 500 mg/dose on week 0, 500 mg/dose on week 2, 500 mg/dose on week 6, and 500 mg/dose on week 10; (iii) 1000 mg/dose on week 0, 1000 mg/dose on week 2, 1000 mg/dose on week 6, and 500 mg/dose on week 10; (iv) 1000 mg/dose on week 0, 1000 mg/dose on week 2, 500 mg/dose on week 6, and 500 mg/dose on week 10; or (v) 1000 mg/dose on week 0, 500 mg/dose on week 2, 500 mg/dose on week 6, and 500 mg/dose on week 10. In yet another embodiment, the first therapeutically effective amount for the TL1A inhibitor in the combination therapy comprises 2000, 1950, 1900, 1850, 1800, 1750, 1700, 1650, 1600, 1550, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, or 200 mg/dose TL1A inhibitor. In a further embodiment, the first therapeutically effective amount for the TL1A inhibitor in the combination therapy comprises 1000 mg/dose every 4 weeks, 500 mg/dose every 4 weeks, 250 mg/dose every 4 weeks, 100 mg/dose every 4 weeks, 1000 mg/dose every 2 weeks, 500 mg/dose every 2 weeks, 250 mg/dose every 2 weeks, or 100 mg/dose every 2 weeks.

The combination of the TL1A inhibitor and the IL23 inhibitor for the methods provided herein, including in this Section (Section 4.7) can be administered with various frequencies, durations, and the total number of administrations as provided in Sections 4.5, 4.6 and 4.7 for TL1A and in Sections 4.3.2, 4.5, and 4.7 for IL23 inhibitors. For example, in one embodiment of the methods of treating an inflammatory disease or condition provided herein, including in this Section (Section 4.7), the administering comprises administering once every 2, 4, 6, 8, 10, or 12 weeks. In another embodiment, the administering comprises administering once every 2 or 4 weeks for the first 2 administrations and then once every 2, 4, 6, or 8 weeks for the remaining administration

Pharmaceutical compositions with the combination of TL1A inhibitor and IL23 inhibitor are also provided herein, which can be used for the various combination therapies provided herein, including in this Section (Section 4.7). In one aspect, provided herein is a pharmaceutical composition comprising a first therapeutically effective amount of an inhibitor of tumor necrosis factor-like protein 1A (“TL1A” and such inhibitor, “TL1A inhibitor”) and a second therapeutically effective amount of an inhibitor of interleukin 23 (“IL23 inhibitor”).

The therapeutically effective amounts for TL1A inhibitors and the therapeutically effective amounts for IL23 inhibitors have been provided above and below. Accordingly, in various embodiments of the pharmaceutical compositions provided herein, including in this Section (Section 4.7), the first therapeutically effective amount of TL1A inhibitor comprises any amount provided for TL1A inhibitor in Sections 4.5, 4.6 and 4.7. In various embodiments of the pharmaceutical compositions provided herein, including in this Section (Section 4.7), the second therapeutically effective amount of IL23 inhibitor comprises any amount provided for IL23 inhibitor in Sections 4.3.2, 4.5, and 4.7. In various embodiments of the pharmaceutical compositions provided herein, including in this Section (Section 4.7), the first therapeutically effective amount of TL1A inhibitor comprises any amount provided for TL1A inhibitor in Sections 4.5, 4.6 and 4.7 and the second therapeutically effective amount of IL23 inhibitor comprises any amount provided for IL23 inhibitor in Sections 4.3.2, 4.5, and 4.7.

Specifically, in one embodiment, the first therapeutically effective amount of TL1A inhibitor in the pharmaceutical composition provided herein, including in this Section (Section 4.7), comprises 2000, 1950, 1900, 1850, 1800, 1750, 1700, 1650, 1600, 1550, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, or 200 mg of TL1A inhibitor.

The TL1A inhibitors and the IL23 inhibitors can be combined in various ratios in the pharmaceutical compositions provided herein, including in this Section (Section 4.7). In one embodiment, the molar ratio of the first therapeutically effective amount of the TL1A inhibitor to the second therapeutically effective amount of the IL23 inhibitor in the pharmaceutical composition is about 100:1, about 95:1, about 90:1, about 85:1, about 80:1, about 75:1, about 70:1, about 65:1, about 60:1, about 55:1, about 50:1, about 45:1, about 40:1, about 35:1, about 30:1, about 25:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, or about 1:100.

The characteristics and/or properties of the TL1A inhibitors have been described in Section 4.3.1 and provided for the pharmaceutical compositions of this Section (Section 4.7). For example, in some embodiments, the TL1A inhibitor is an inhibitor of TL1A expression or an inhibitor of TL1A activity. In some other embodiments, the TL1A inhibitor is an anti-TL1A antibody or antigen binding fragment thereof. In one embodiment, the TL1A inhibitor binds to both monomeric TL1A and trimeric TL1A. In other embodiments, the TL1A inhibitor comprises an anti-TL1A antibody or antigen binding fragment thereof and the antibody or antigen binding fragment binds to both monomeric TL1A and trimeric TL1A. In one embodiment, the TL1A inhibitor comprises an anti-TL1A antibody or antigen binding fragment thereof, the antibody or antigen binding fragment binds to both monomeric TL1A and trimeric TL1A, and the TL1A inhibitor blocks interaction of TL1A to Death Receptor 3 (“DR3”). In one embodiment, the TL1A inhibitor blocks interaction of TL1A to Death Receptor 3 (“DR3”). In one embodiment, the TL1A inhibitor comprises an anti-TL1A antibody or antigen binding fragment thereof and the TL1A inhibitor blocks interaction of TL1A to Death Receptor 3 (“DR3”). In another embodiment, the TL1A inhibitor the TL1A inhibitor comprises an anti-TL1A antibody or antigen binding fragment thereof and binding affinity of the antibody or antigen binding fragment to monomeric TL1A as measured by dissociation equilibrium constant (K_{D-monomer}) is comparable to binding affinity of the antibody or antigen binding fragment to trimeric TL1A as measured by dissociation equilibrium constant (K_{D-trimer}). In yet another embodiment, the K_{D-monomer} is within 1.5, 2, 3, 4, 5, 6, 7, 8, 9, or 10 fold of the K_{D-trimer}. In a further embodiment, the K_{D-monomer} is no more than 0.06 nM. In one embodiment, the K_{D-trimer} is no more than 0.06 nM. In yet another embodiment, the K_{D-monomer} is no more than 0.06 nM and the K_{D-trimer} is no more than 0.06 nM.

The chemical and sequence characteristics of the TL1A inhibitors have been described in Section 4.3.1 and provided for the methods of treating an inflammatory disease or condition and the pharmaceutical compositions this Section (Section 4.7). For example, in various embodiments of the methods of treating an inflammatory disease or condition and/or the pharmaceutical compositions provided herein, including in this Section (Section 4.7), the anti-TL1A antibody or antigen binding fragment in the combination therapy comprises a heavy chain variable region comprising: an HCDR1 comprising an amino acid sequence set forth by SEQ ID NO: 1, an HCDR2 comprising an amino acid sequence set forth by any one of SEQ ID NOS: 2-5, and an HCDR3 comprising an amino acid sequence set forth by any one of SEQ ID NOS: 6-9; and a light chain variable region comprising an LCDR1 comprising an amino acid sequence set forth by SEQ ID NO: 10, an LCDR2 comprising an amino acid sequence set forth by SEQ ID NO: 11, an LCDR3 comprising an amino acid sequence set forth by any one of SEQ ID NOS: 12-15. In some embodiments of the methods of treating an inflammatory disease and/or condition or the pharmaceutical compositions provided herein, including in this Section (Section 4.7), the anti-TL1A antibody or antigen binding fragment in the combination therapy comprises a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise no or fewer than nine amino acid modification(s) from the human IGHV1-46*02 framework and the human IGKV3-20 framework. In certain embodiments of the methods of treating an inflammatory disease and/or condition or the pharmaceutical compositions provided herein, including in this Section (Section 4.7), the anti-TL1A antibody or antigen binding fragment in the combination therapy comprises a heavy chain variable domain comprising an amino acid sequence at least 96% identical to any one of SEQ ID NOS: 101-169, and a light chain variable domain comprising an amino acid sequence at least 96% identical to any one of SEQ ID NOS: 201-220. In certain embodiments of the methods of treating an inflammatory disease and/or condition or the pharmaceutical compositions provided herein, including in this Section (Section 4.7), the anti-TL1A antibody or antigen binding fragment in the combination therapy comprises a heavy chain variable region comprising SEQ ID NO: 301 XIVQLVQSGAEVKKPGASVKVSCKAS[HCDR1]WVX2QX3PGQGLEWX4G[HCDR2]RX5TX6TX7DTSTSTX8YX9ELSSLRSEDTAVYYCAR[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRFSGSGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V, wherein HCDR1 comprises an amino acid sequence set forth by SEQ ID NO: 1, HCDR2 comprises an amino acid sequence set forth by any one of SEQ ID NOS: 2-5, HCDR3 comprises an amino acid sequence set forth by any one of SEQ ID NOS: 6-9, LCDR1 comprises an amino acid sequence set forth by SEQ ID NO: 10, LCDR2 comprises an amino acid sequence set forth by SEQ ID NO: 11, and LCDR3 comprises an amino acid sequence set forth by any one of SEQ ID NOS: 12 or 13.

Similarly, the chemical, functional and sequence characteristics of the IL23 inhibitors have been described in Section 4.3.2 and provided for the methods of treating an inflammatory disease or condition and the pharmaceutical compositions this Section (Section 4.7). For example, in various embodiments of the methods of treating an inflammatory disease or condition and/or the pharmaceutical compositions provided herein, including in this Section (Section 4.7), the IL23 inhibitor specifically inhibits IL23. In some embodiments of the methods of treating an inflammatory disease or condition and/or the pharmaceutical compositions provided herein, including in this Section (Section 4.7), the IL23 inhibitor comprises an antagonistic antibody or antigen-binding fragment against IL23. In some embodiments of the methods of treating an inflammatory disease or condition and/or the pharmaceutical compositions provided herein, including in this Section (Section 4.7), the IL23 inhibitor comprises ustekinumab. In some embodiments, the second therapeutically effective amount for ustekinumab comprises 45 mg/dose if the subject has a body weight of less than or equal to 100 kg. In some embodiments, the second therapeutically effective amount for ustekinumab comprises 90 mg/dose if the subject has a body weight of greater than 100 kg. In some embodiments, the second therapeutically effective amount for ustekinumab comprises 45 mg/dose. In some embodiments, the second therapeutically effective amount for ustekinumab comprises 90 mg/dose.

In some embodiments of the methods of treating an inflammatory disease or condition and/or the pharmaceutical compositions provided herein, including in this Section (Section 4.7), the IL23 inhibitor comprises guselkumab. In some embodiments, the second therapeutically effective amount for guselkumab comprises a dose of 100 mg administered in an initial dose, 4 weeks after the initial dose and every 8 weeks after the dose at 4 weeks. In some embodiments, the second therapeutically effective amount for guselkumab comprises a dose of 100 mg administered at week 0, week 4, and every 8 weeks after the dose at week 4. In some embodiments of the methods of treating an inflammatory disease or condition and/or the pharmaceutical compositions provided herein, including in this Section (Section 4.7), the IL23 inhibitor comprises risankizumab. In some embodiments, the second therapeutically effective amount for risankizumab comprises a dose of 150 mg by subcutaneous injection at Week 0, Week 4, and every 12 weeks thereafter.

In some embodiments of the methods of treating an inflammatory disease or condition and/or the pharmaceutical compositions provided herein, including in this Section (Section 4.7), the IL23 inhibitor comprises brazikumab. In some embodiments, the second therapeutically effective amount for brazikumab comprises (a) 720-1440 mg on or about days 1, 29, and 57 delivered intravenously, followed by (b) about 240 mg delivered subcutaneously on or about day 85 and about every 4 weeks thereafter through at least week 48.

In some embodiments of the methods of treating an inflammatory disease or condition and/or the pharmaceutical compositions provided herein, including in this Section (Section 4.7), the IL23 inhibitor comprises mirikizumab. In some embodiments, the second therapeutically effective amount for mirikizumab comprises at least one induction dose of about 200 mg to about 1200 mg of the mirikizumab and at least one maintenance dose of about 100 mg to about 600 mg of the mirikizumab.

In some embodiments of the methods of treating an inflammatory disease or condition and/or the pharmaceutical compositions provided herein, including in this Section (Section 4.7), the IL23 inhibitor comprises tildrakizumab. In some embodiments, the second therapeutically effective amount for tildrakizumab comprises a dose of 100 mg of the tildrakizumab at Weeks 0, 4, and every twelve weeks thereafter up to 52 week. In some embodiments, the second therapeutically effective amount for tildrakizumab comprises a dose of 100 mg of the tildrakizumab at Weeks 0, 4, and every twelve weeks thereafter.

In some embodiments of the methods of treating an inflammatory disease or condition and/or the pharmaceutical compositions provided herein, including in this Section (Section 4.7), the IL23 inhibitor comprises briakinumab. In some embodiments, the second therapeutically effective amount for briakinumab comprises (i) a first dose amount of 180 mg to 220 mg of the antibody or antigen-binding domain thereof, at week 0, and for the same first dose amount of the antibody or antigen-binding domain thereof at week 4, and (ii) a second dose amount of 80 mg to 120 mg of the antibody or antigen-binding domain thereof every 4 weeks thereafter. In some embodiments, the second therapeutically effective amount for briakinumab comprises (i) a first dose amount of 180 mg to 220 mg of the antibody or antigen-binding domain thereof, at week 0, and for the same first dose amount of the antibody or antigen-binding domain thereof at week 4, and (ii) a second dose amount of 80 mg to 120 mg of the antibody or antigen-binding domain thereof every 4 weeks thereafter up to 52 weeks.

In some embodiments of the methods of treating an inflammatory disease or condition and/or the pharmaceutical compositions provided herein, including in this Section (Section 4.7), the second therapeutically effective amount for an IL23 inhibitor is the amount, the dose, and/or dosing regimen provided in Section 4.3.2 for the IL23 inhibitor.

In various embodiments of the methods of treating an inflammatory disease or condition provided herein, including in this Section (Section 4.7), the third therapeutically effective amount for the TL1A inhibitor is identical to the first therapeutically effective amount for the TL1A inhibitor. In certain embodiments of the methods of treating an inflammatory disease or condition provided herein, including in this Section (Section 4.7), the third therapeutically effective amount for the TL1A inhibitor is less than the first therapeutically effective amount for the TL1A inhibitor.

In various embodiments of the methods of treating an inflammatory disease or condition provided herein, including in this Section (Section 4.7), the fourth therapeutically effective amount for the IL23 inhibitor is identical to the second therapeutically effective amount for the IL23 inhibitor. In certain embodiments of the methods of treating an inflammatory disease or condition provided herein, including in this Section (Section 4.7), the fourth therapeutically effective amount for the IL23 inhibitor is less than the second therapeutically effective amount for the IL23 inhibitor.

In various embodiments of the methods of treating an inflammatory disease or condition and/or the pharmaceutical compositions provided herein, including in this Section (Section 4.7), wherein the dose is one dose per day.

In various embodiments of the methods of treating an inflammatory disease or condition and/or the pharmaceutical compositions provided herein, including in this Section (Section 4.7), a subject refers to any animal, including, but not limited to, humans, non-human primates, rodents, and domestic and game animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various embodiments, the subject previously diagnosed with or identified as suffering from or having a condition may or may not have undergone treatment for a condition. In some embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition. In some embodiments, the subject is a “patient,” that has been diagnosed with a disease or condition described herein. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In various embodiments of the methods of treating an inflammatory disease or condition and/or the pharmaceutical compositions provided herein, including in this Section (Section 4.7), the subject is a human subject. In certain embodiments of the methods of treating an inflammatory disease or condition and/or the pharmaceutical compositions provided herein, including in this Section (Section 4.7), the subject is a patient with an inflammatory disease or condition. In certain embodiments of the methods of treating an inflammatory disease or condition and/or the pharmaceutical compositions provided herein, including in this Section (Section 4.7), the subject is a patient with inflammatory bowel disease. In certain embodiments of the methods of treating an inflammatory disease or condition and/or the pharmaceutical compositions provided herein, including in this Section (Section 4.7), the subject is a patient with UC. In certain embodiments of the methods of treating an inflammatory disease or condition and/or the pharmaceutical compositions provided herein, including in this Section (Section 4.7), the subject is a patient with CD.

As is clear from the descriptions above and below, the TL1A inhibitor and/or IL23 inhibitor in the combination therapy is formulated in pharmaceutical compositions as described in Sections 4.3, 4.5, and 4.6. In various embodiments of the methods of treating an inflammatory disease or condition and/or the pharmaceutical compositions provided herein, including in this Section (Section 4.7), the TL1A inhibitor in the combination therapy is formulated in pharmaceutical compositions as described in Sections 4.3, 4.5, and 4.6. In various embodiments of the methods of treating an inflammatory disease or condition and/or the pharmaceutical compositions provided herein, including in this Section (Section 4.7), the IL23 inhibitor in the combination therapy is formulated in pharmaceutical compositions as described in Sections 4.3, 4.5, and 4.6. In various embodiments of the methods of treating an inflammatory disease or condition and/or the pharmaceutical compositions provided herein, including in this Section (Section 4.7), the TL1A inhibitor in the combination therapy is formulated in pharmaceutical compositions as described in Sections 4.3, 4.5, and 4.6 and the IL23 inhibitor in the combination therapy is formulated in pharmaceutical compositions as described in Sections 4.3, 4.5, and 4.6.

For various embodiments of the methods provided herein, including in this Section (Section 4.7), for example those of the preceding paragraphs), embodiments of the TL1A inhibitors are further provided in Section 4.3.1 (including, for example, (i) embodiments for anti-TL1A antibodies with exemplary CDRs, framework sequences, constant region sequences, Fc mutations, variable regions, Fc regions, and other properties, as described in Section 4.3.1(a) and embodiments for soluble DR3 protein, a variant of soluble DR3 protein, a soluble DR3 protein fused with Fc, or a variant of soluble DR3 protein fused with Fc, each as described in Section 4.3.1(c)); embodiments of the IL23 inhibitors are further provided in Section 4.3.2; assays for screening, testing, and validating the anti-TL1A antibodies and/or anti-IL23 antibodies are provided in Section 4.3.3; methods for generating, improving, mutating, cloning, expressing, and isolating the anti-TL1A antibodies and or anti-IL23 antibodies are provided in Section 4.4; pharmaceutical compositions for the TL1A inhibitors and pharmaceutical compositions for the IL23 inhibitors are described and provided in Section 4.5; pharmaceutical compositions comprising both the TL1A inhibitors and the IL23 inhibitors are described and provided in Section 4.7; therapeutically effective amount including the dose and dosing regimens for the TL1A inhibitors in the combination therapies are further provided in Sections 4.5, 4.6, 4.7, and 5; therapeutically effective amount including the dose and dosing regimens for the IL23 inhibitors in the combination therapies are further provided in Sections 4.3.2, 4.5, 4.7, and 5; further specific and validated embodiments for the TL1A inhibitors, the IL23 inhibitors and the methods of using the combination of TL1A inhibitors and IL23 inhibitors are provided in Section 5. As such, the disclosure provides the various combinations of the TL1A inhibitors, IL23 inhibitors, the pharmaceutical compositions of such TL1A inhibitors and/or IL23 inhibitors, the methods of generating the TL1A and/or IL23 inhibitors, the methods of assaying the TL1A inhibitors and/or IL23 inhibitors, and the methods of using the combination of TL1A inhibitors and IL23 inhibitors for treating inflammatory diseases and conditions.

### 7. EXAMPLES

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1: Design of Humanized Anti-TL1A Antibodies

Two different strategies were employed to identify humanized variants that express well in mammalian cells, preserve TL1A binding, and display high monomeric content.

The first strategy utilized a previously humanized variant, termed ASX, that displays high monomeric content (98%) and expresses well (30 μg/mL in small-scale transient cultures) as a template for additional mutagenesis. However, ASX contains a significant number of murine framework residues, eight heavy chain residues and 7 light chain residues, which may pose an immunogenicity risk. The ASX heavy and light chain templates were used to systematically mutate murine framework residues to human residues corresponding to the most closely related human germline framework. The goal of this strategy was to reduce the total number of murine framework residues while preserving the favorable expression and solubility characteristics of ASX. Because ASX contained 15 murine framework residues there were 2\hat{ }15 (32,768) distinct variants (restricting each position to either the murine or the human residue) that could be made and tested.

The second strategy utilized a previously humanized variant, termed c34, that expresses well (17 μg/mL in small-scale transient cultures) and contains CDRs optimized for binding within a fully human germline framework, as a template for additional mutagenesis. Large-scale expression of c34 unexpectedly resulted in a sub-optimal monomeric content (55-60%). The c34 heavy and light chain templates were used to systematically mutate certain framework residues to murine residues corresponding to the original murine antibody framework. The goal of this strategy was to improve the solubility of c34 (monomeric content) through the introduction of as few murine framework residues as possible (minimizing potential immunogenicity risks) while preserving the favorable expression characteristics of c34.

For both strategies, the initial approach was to scan differing framework residues, one at a time, and express and characterize the variants. Thus, human framework residues were introduced into variant ASX where it differed from c34 and conversely, murine framework mutations were introduced into variant c34 where it differed from ASX. The initial scan identified certain framework and CDR residues that had minimal impact on the characteristics displayed by the template antibody while other mutations had a more dramatic impact, favorable in some cases and unfavorable in others. The information gained from the positional scan was subsequently used in an iterative and combinatorial fashion, to identify multiple variants with favorable characteristics. Importantly, by applying a stepwise, iterative and combinatorial approach the beneficial variants were identified without necessitating the expression and characterization of 32,768 distinct variants.

In certain cases, mutation of the first residue of the heavy chain from glutamine to aspartic acid or glutamic acid was evaluated, alone or in combination with other mutations.

In addition, for both strategies certain CDR residues were also mutated to determine the impact on expression and solubility. For example, a limited number of mutations in HCDR2, HCDR3 and LCDR3 were examined. Similar to the approach used with frameworks, the mutations were predominantly restricted to the original murine CDR residue or mutations that were previously identified as enhancing binding affinity.

Finally, for both strategies “shuffling” of heavy and light chains was used. Specifically, certain human light chains containing few murine framework residues and having a favorable impact on expression of antibody with higher monomeric content were identified early in the process and these were paired with various engineered heavy chains in order to accelerate the process of identifying suitable variants.

Examples of certain designed antibodies are shown in Table 1.

TABLE 1
Variable region sequences of select anti-TL1A Antibodies

| | Heavy Chain Variable Region | Light Chain Variable Region |
|---|---|---|
| Antibody | SEQ ID NO | SEQ ID NOS |
| A15 | 108 | 203 |
| A29 | 108 | 205 |
| A30 | 108 | 204 |
| A31 | 136 | 205 |
| A32 | 137 | 205 |
| A33 | 137 | 202 |
| A34 | 107 | 208 |
| A35 | 138 | 208 |
| A36 | 139 | 208 |
| A37 | 140 | 208 |
| A38 | 141 | 208 |
| A39 | 142 | 208 |
| A40 | 143 | 208 |
| A41 | 115 | 208 |
| A42 | 144 | 208 |
| A43 | 145 | 208 |
| A44 | 146 | 208 |
| A45 | 120 | 208 |
| A46 | 147 | 208 |
| A47 | 148 | 208 |
| A48 | 108 | 210 |
| A49 | 108 | 211 |
| A50 | 108 | 212 |
| A51 | 108 | 213 |
| A52 | 108 | 214 |
| A53 | 146 | 208 |
| A54 | 149 | 208 |
| A55 | 109 | 208 |
| A56 | 108 | 215 |
| A57 | 150 | 202 |
| A58 | 125 | 202 |
| A59 | 117 | 202 |
| A60 | 151 | 202 |
| A61 | 152 | 202 |
| A62 | 153 | 202 |
| A63 | 154 | 202 |
| A64 | 121 | 202 |
| A65 | 128 | 202 |
| A66 | 155 | 202 |
| A67 | 122 | 202 |
| A68 | 123 | 202 |
| A69 | 156 | 202 |
| A70 | 157 | 202 |
| A71 | 158 | 202 |
| A72 | 131 | 202 |
| A73 | 157 | 205 |
| A74 | 158 | 205 |
| A75 | 131 | 205 |
| A76 | 159 | 202 |
| A77 | 160 | 202 |
| A78 | 124 | 202 |
| A79 | 107 | 208 |
| A81 | 139 | 208 |
| A82 | 140 | 208 |
| A83 | 144 | 208 |
| A85 | 136 | 209 |
| A86 | 136 | 216 |
| A87 | 136 | 217 |
| A88 | 136 | 218 |
| A89 | 136 | 219 |
| A90 | 136 | 220 |
| A91 | 133 | 202 |
| A92 | 161 | 202 |
| A93 | 162 | 202 |
| A94 | 124 | 202 |
| A95 | 131 | 205 |
| A96 | 128 | 205 |
| A97 | 121 | 202 |
| A98 | 122 | 202 |
| A99 | 123 | 202 |
| A100 | 107 | 204 |
| A101 | 140 | 204 |
| A102 | 115 | 204 |
| A103 | 120 | 204 |
| A104 | 139 | 204 |
| A105 | 143 | 204 |
| A107 | 108 | 202 |
| A108 | 156 | 205 |
| A109 | 133 | 205 |
| A110 | 125 | 205 |
| A111 | 150 | 205 |
| A112 | 117 | 205 |
| A113 | 124 | 205 |
| A114 | 121 | 205 |
| A115 | 122 | 205 |
| A116 | 123 | 205 |
| A117 | 151 | 205 |
| A118 | 153 | 205 |
| A119 | 159 | 205 |
| A120 | 154 | 205 |
| A121 | 163 | 204 |
| A122 | 113 | 204 |
| A123 | 112 | 204 |
| A124 | 164 | 204 |
| A125 | 105 | 204 |
| A126 | 114 | 204 |
| A127 | 118 | 204 |
| A128 | 111 | 204 |
| A129 | 110 | 204 |
| A130 | 121 | 205 |
| A132 | 128 | 206 |
| A133 | 121 | 206 |
| A134 | 122 | 206 |
| A135 | 133 | 206 |
| A136 | 125 | 206 |
| A137 | 121 | 207 |
| A138 | 122 | 207 |
| A139 | 110 | 207 |
| A140 | 110 | 202 |
| A141 | 111 | 207 |
| A142 | 111 | 202 |
| A143 | 136 | 202 |
| A144 | 111 | 204 |
| A145 | 133 | 201 |
| A146 | 125 | 20 |
| A147 | 117 | 201 |
| A148 | 121 | 201 |
| A149 | 122 | 201 |
| A150 | 128 | 201 |
| A151 | 124 | 201 |
| A152 | 131 | 201 |
| A153 | 133 | 205 |
| A154 | 125 | 205 |
| A155 | 121 | 205 |
| A156 | 122 | 205 |
| A157 | 104 | 204 |
| A158 | 10 | 204 |
| A159 | 119 | 204 |
| A160 | 102 | 204 |
| A161 | 165 | 204 |
| A162 | 106 | 204 |
| A163 | 166 | 204 |
| A164 | 167 | 204 |
| A165 | 139 | 205 |
| A166 | 146 | 205 |
| A167 | 120 | 205 |
| A168 | 147 | 205 |
| A169 | 126 | 205 |
| A170 | 135 | 205 |
| A171 | 168 | 205 |
| A172 | 130 | 205 |
| A173 | 127 | 205 |
| A174 | 132 | 205 |
| A175 | 126 | 201 |
| A176 | 135 | 201 |
| A177 | 168 | 201 |
| A178 | 130 | 201 |
| A179 | 127 | 201 |
| A180 | 132 | 201 |
| A181 | 107 | 202 |
| A182 | 138 | 202 |
| A183 | 140 | 202 |
| A184 | 145 | 202 |
| A185 | 147 | 202 |
| A186 | 144 | 202 |
| A187 | 120 | 202 |
| A188 | 115 | 202 |
| A189 | 146 | 202 |
| A190 | 141 | 202 |
| A191 | 142 | 202 |
| A192 | 143 | 202 |
| A193 | 109 | 205 |
| A194 | 103 | 205 |
| A195 | 169 | 205 |
| A196 | 129 | 205 |
| A197 | 116 | 205 |
| A198 | 134 | 205 |
| A199 | 109 | 201 |
| A200 | 103 | 201 |
| A201 | 169 | 201 |
| A202 | 129 | 201 |
| A203 | 116 | 201 |
| A204 | 134 | 201 |
| A205 | 109 | 202 |
| A206 | 103 | 202 |
| A207 | 169 | 202 |
| A208 | 129 | 202 |
| A209 | 116 | 202 |
| A210 | 134 | 202 |
| A211 | 108 | 201 |
| A212 | 107 | 201 |
| A213 | 106 | 201 |
| A214 | 111 | 201 |
| A215 | 110 | 201 |
| A216 | 112 | 201 |
| A217 | 101 | 201 |
| A218 | 119 | 201 |
| A219 | 104 | 201 |
| A220 | 102 | 205 |
| A221 | 105 | 201 |
| A222 | 114 | 201 |
| A223 | 103 | 202 |
| A224 | 116 | 201 |
| A500 | 301 | 303 |
| A501 | 302 | 303 |

As used herein, reference to A (number), refers to an antibody of this table. For instance, A15 used herein refers to A15 in Table 1.

### Example 2: Generation and Characterization of Humanized Anti-TL1A Antibodies

Humanized anti-TL1A antibodies designed in Example 1 were prepared and characterized.

### Cloning of Humanized Antibodies

DNA encoding leader sequence and the heavy and light chain variable regions of humanized variants of interest was cloned into pFuse1-hIgG1-Fc1 (InvivoGen) and pFuse2-CLig-hk (InvivoGen), respectively. Two distinct humanized heavy chain templates, termed ASX-HC and c34-HC, and four distinct humanized light chain templates, termed ASX-LC, cH3-1, c34-LC, cXL3-13-LC and cXL3-15-LC were all cloned.

In order to introduce mutations into the templates, the QuickChange Site Directed Mutagenesis Kit (Agilent, cat. #200518) was used per manufacturer's directions. Briefly, mutagenesis was performed using miniprep double-stranded plasmid DNA, two synthetic oligonucleotides primers containing the desired mutation, PfuTurbo® DNA polymerase and a temperature cycler. Following temperature cycling, the product was treated with Dpn I. The nicked vector DNA containing the mutation(s) of interest was used to transform bacteria. Subsequently, colonies were picked, the DNA was sequenced to confirm mutagenesis and was subsequently used for transfection of mammalian FreeStyle 293-F cells.

### Antibody Expression

Small-scale (3 mL, 6-well) expression of variants in FreeStyle 293-F cells was performed in the following manner. One or two days prior to transfection cells were passaged so that the density would be >1×10⁶ cells/mL on the day of the transfection. Typically, this meant passaging at 6-7×10⁵ cells/mL one day prior or 4×10⁵ cells/mL two days prior. Transfections were only performed with cell viability>90%. On the day of the transfection Opti-MEM media was warmed to 37° C. and cells were resuspended to 1.1×10⁶ cells/mL, using 3.3×10⁶ cells per 3 mL transfection. A total of 3 μg DNA was used for each transfection. Briefly, the transfections used heavy and light chain plasmid at a heavy chain: light chain ratio of 1:3. For 3 mL transfections, 4 μL 293fectin was added to 96 μL Opti-MEM, combined with 100 μL DNA mixture, and incubated at 25° C. for 20-30 minutes. Subsequently, this mixture was added dropwise to 2.8 mL cells and the plate was transferred to an incubator and placed on a rotating platform at 175 rpm for up to 120 hours. After 96-120 hours, transfection supernatants were collected by centrifuging the transfected cells and supernatant at 1200 rpm for 5 min. The supernatant was transferred to a clean tube and centrifuged again at 3900 rpm for 10 min to remove any remaining cell debris. The supernatant was filtered through a 0.45 mm PES syringe filter and stored at 4° C. until the next step.

### Quantitation of Antibody Expression

Antibody expression was quantitated by ELISA. Briefly, a Corning Costar 3366 96-well round bottom high bind plate was coated with 50 mL anti-kappa (2 μg/mL) in PBS overnight at 4° C. The plate was washed 3× with PBS-0.05% Tween 20 (PBS-T) and was blocked with 100 μL 1% BSA/PBS for 1 h at 25° C. The block was removed, and culture supernatant diluted 5-fold was added and serially diluted 2-fold across the plate. Every plate also contained an IgG standard diluted serially 3-fold beginning at 1 μg/mL. Samples were incubated for 1 h at 25° C., the plate was washed three times with PBS-T, and 50 μL anti-Fc HRP secondary (Southern Biotech #2048-05), diluted 1:4000 in BSA/PBS was added for 1 h at 25° C. The plate was washed three times with PBS-T and developed for up to 15 min following the addition of 50 μL Ultra TMB ELISA substrate (Thermo #34028). The reaction was terminated by the addition of 50 μL 2 N H₂SO₄ and the A450 nm was measured. Antibody expression levels obtained from 3 mL scale transfections are shown in Table 2.

TABLE 2
Expression, binding, and analytical SEC characterization
of anti-TL1A antibodies (ND, not determined)

| Var- | Expression | KD | % | Murine | HC | LC |
|---|---|---|---|---|---|---|
| iant | (μg/mL) | (pM) | Monomer | FR | Template | Template |
| 15 | 21 | ND | 87 | 8 | ASX | cH3-1 |
| 29 | 18 | 65 | 65 | 10 | ASX | c34 |
| 30 | 29 | 77 | 90 | 8 | ASX | cXL3-13 |
| 31 | 11 | 92 | 73 | 2 | c34 | c34 |
| 32 | 10 | 111 | 78 | 2 + D | c34 | c34 |
| 33 | 21 | 81 | 54 | 0 + D | c34 | c34 |
| 34 | 35 | <50 | 97 | 14 | ASX | ASX |
| 35 | 36 | 72 | 91 | 14 | ASX | ASX |
| 36 | 40 | <50 | 87 | 13 | ASX | ASX |
| 37 | 40 | 34 | 95 | 14 | ASX | ASX |
| 38 | 28 | 103 | 75 | 14 | ASX | ASX |
| 39 | 15 | 125 | 83 | 14 | ASX | ASX |
| 40 | 30 | <50 | 87 | 13 | ASX | ASX |
| 41 | 20 | 16 | 96 | 14 | ASX | ASX |
| 42 | 30 | <50 | 88 | 14 | ASX | ASX |
| 43 | 18 | 51 | 90 | 14 | ASX | ASX |
| 44 | ND | ND | ND | 13 | ASX | ASX |
| 45 | 15 | 85 | 90 | 13 | ASX | ASX |
| 46 | 27 | 63 | 72 | 13 | ASX | ASX |
| 47 | 18 | 82 | 78 | 12 | ASX | ASX |
| 48 | 22 | 76 | 92 | 14 | ASX | ASX |
| 49 | 26 | 92 | 65 | 13 | ASX | ASX |
| 50 | 33 | 19 | 94 | 14 | ASX | ASX |
| 51 | 16 | <50 | 93 | 14 | ASX | ASX |
| 52 | 29 | 27 | 91 | 13 | ASX | ASX |
| 53 | 26 | 126 | 84 | 13 | ASX | ASX |
| 54 | 25 | 83 | 94 | 15 +D | ASX | ASX |
| 55 | 22 | 91 | 99 | 15 +E | ASX | ASX |
| 56 | 15 | 116 | 71 | 14 | ASX | ASX |
| 57 | 20 | 191 | 59 | 1 | c34 | c34 |
| 58 | 9 | 112 | 67 | 1 | c34 | c34 |
| 59 | 11 | 136 | 78 | 2 | c34 | c34 |
| 60 | 19 | 168 | 57 | 0 | c34 | c34 |
| 61 | 15 | 127 | 44 | 1 | c34 | c34 |
| 62 | 21 | 150 | 58 | 1 | c34 | c34 |
| 63 | 20 | 132 | 52 | 0 | c34 | c34 |
| 64 | 2 | 90 | 97 | 0 | c34 | c34 |
| 65 | 7 | 97 | 69 | 1 | c34 | c34 |
| 66 | 19 | 150 | 49 | 1 | c34 | c34 |
| 67 | 4 | 89 | 97 | 1 | c34 | c34 |
| 68 | 2 | 74 | 92 | 1 | c34 | c34 |
| 69 | 12 | 136 | 64 | 0 + E | c34 | c34 |
| 70 | 15 | 149 | 54 | 1 | c34 | c34 |
| 71 | 18 | 150 | 55 | 2 | c34 | c34 |
| 72 | 13 | 159 | 61 | 3 | c34 | c34 |
| 73 | 8 | 128 | 71 | 3 | c34 | c34 |
| 74 | 10 | 141 | 70 | 4 | c34 | c34 |
| 75 | 8 | 259 | 95 | 5 | c34 | c34 |
| 76 | 19 | ND | 50 | 0 | c34 | c34 |
| 77 | 12 | ND | 50 | 2 | c34 | c34 |
| 78 | 3 | ND | 86 | 2 | c34 | c34 |
| 79 | 42 | ND | 98 | 14 | ASX | ASX |
| 81 | 31 | ND | 88 | 13 | ASX | ASX |
| 82 | 26 | ND | 92 | 14 | ASX | ASX |
| 83 | 29 | ND | 74 | 14 | ASX | ASX |
| 85 | 25 | 130 | 49 | 1 | c34 | c34 |
| 86 | 26 | 129 | 55 | 1 | c34 | c34 |
| 87 | 26 | 121 | 52 | 1 | c34 | c34 |
| 88 | 9 | 81 | 63 | 2 | c34 | c34 |
| 89 | 31 | 117 | 55 | 1 | c34 | c34 |
| 90 | 19 | 107 | 53 | 1 | c34 | c34 |
| 91 | 14 | 132 | 63 | 1 | c34 | c34 |
| 92 | 20 | 121 | 49 | 1 | c34 | c34 |
| 93 | 12 | 117 | 63 | 2 | c34 | c34 |
| 94 | 5 | 81 | 91 | 2 | c34 | c34 |
| 95 | 13 | 105 | 92 | 5 | c34 | c34 |
| 96 | 7 | 95 | 99 | 3 | c34 | c34 |
| 97 | 2 | 71 | 97 | 0 | c34 | c34 |
| 98 | 7 | 140 | 98 | 1 | c34 | c34 |
| 99 | 3 | 102 | 95 | 1 | c34 | c34 |
| 100 | 39 | 84 | 84 | 7 | ASX | cXL3-13 |
| 101 | 23 | 96 | 81 | 7 | ASX | cXL3-13 |
| 102 | 19 | 104 | 75 | 7 | ASX | cXL3-13 |
| 103 | 11 | 107 | 90 | 6 | ASX | cXL3-13 |
| 104 | 26 | 108 | 70 | 6 | ASX | cXL3-13 |
| 105 | 23 | 110 | 58 | 6 | ASX | cXL3-13 |
| 107 | 55 | 71 | 85 | 8 | ASX | c34 |
| 108 | 9 | 55 | 83 | 2 + E | c34 | c34 |
| 109 | 9 | 50 | 96 | 3 | c34 | c34 |
| 110 | 7 | 56 | 95 | 3 | c34 | c34 |
| 111 | 17 | 68 | 61 | 3 | c34 | c34 |
| 112 | 6 | 54 | 93 | 4 | c34 | c34 |
| 113 | 2 | 50 | 99 | 4 | c34 | c34 |
| 114 | 1 | 51 | 99 | 2 | c34 | c34 |
| 115 | 3 | 58 | 99 | 3 | c34 | c34 |
| 116 | 1 | 53 | 99 | 3 | c34 | c34 |
| 117 | 16 | 94 | 80 | 2 | c34 | c34 |
| 118 | 21 | 83 | 70 | 3 | c34 | c34 |
| 119 | 15 | 87 | 77 | 2 | c34 | c34 |
| 120 | 12 | 85 | 64 | 2 | c34 | c34 |
| 121 | 24 | 106 | 77 | 6 | ASX | cXL3-13 |
| 122 | 22 | 112 | 85 | 6 | ASX | cXL3-13 |
| 123 | 18 | 104 | 76 | 5 | ASX | cXL3-13 |
| 124 | 21 | 91 | 83 | 6 | ASX | cXL3-13 |
| 125 | 10 | 116 | 98 | 6 | ASX | cXL3-13 |
| 126 | 4 | 123 | 99 | 5 | ASX | cXL3-13 |
| 127 | 8 | 70 | 94 | 6 | ASX | cXL3-13 |
| 128 | 17 | 111 | 84 | 4 | ASX | cXL3-13 |
| 129 | 17 | 99 | 92 | 5 | ASX | cXL3-13 |
| 130 | 1 | 75 | 99 | 2 | c34 | c34 |
| 132 | 6 | 62 | 99 | 2 | c34 | cXL3-13 |
| 133 | 1 | 58 | 99 | 1 | c34 | cXL3-13 |
| 134 | 3 | 55 | 99 | 2 | c34 | cXL3-13 |
| 135 | 7 | 56 | 74 | 2 | c34 | cXL3-13 |
| 136 | 6 | 53 | 84 | 2 | c34 | cXL3-13 |
| 137 | 2 | 50 | 96 | 0 | c34 | cXL3-15 |
| 138 | 5 | 69 | 99 | 1 | c34 | cXL3-15 |
| 139 | 35 | 74 | 78 | 5 | ASX | cXL3-15 |
| 140 | 26 | 73 | 75 | 5 | ASX | c34 |
| 141 | 27 | 108 | 81 | 4 | ASX | cXL3-15 |
| 142 | 25 | 126 | 68 | 4 | ASX | c34 |
| 143 | 16 | 85 | 57 | 0 | c34 | c34 |
| 144 | ND | ND | ND | 4 | ASX | cXL3-13 |
| 145 | 20 | 70 | 78 | 2 | c34 | c34 |
| 146 | 25 | 65 | 84 | 2 | c34 | c34 |
| 147 | 26 | 63 | 87 | 3 | c34 | c34 |
| 148 | 2 | 46 | 98 | 1 | c34 | c34 |
| 149 | 7 | 48 | 99 | 2 | c34 | c34 |
| 150 | 15 | 59 | 83 | 2 | c34 | c34 |
| 151 | 5 | 57 | 96 | 3 | c34 | c34 |
| 152 | 36 | 58 | 73 | 4 | c34 | c34 |
| 153 | 9 | 49 | 97 | 3 | c34 | c34 |
| 154 | 8 | 66 | 92 | 3 | c34 | c34 |
| 155 | 1 | 67 | 99 | 2 | c34 | c34 |
| 156 | 2 | 94 | 99 | 3 | c34 | c34 |
| 157 | 6 | 69 | 93 | 4 | ASX | cXL3-13 |
| 158 | 6 | 66 | 91 | 3 | ASX | cXL3-13 |
| 159 | 4 | 69 | 99 | 4 | ASX | cXL3-13 |
| 160 | 7 | 94 | 99 | 4 | ASX | cXL3-13 |
| 161 | 11 | 72 | 59 | 4 | ASX | cXL3-13 |
| 162 | 9 | 75 | 79 | 3 | ASX | cXL3-13 |
| 163 | 22 | 51 | 60 | 4 | ASX | cXL3-13 |
| 164 | 23 | 58 | 61 | 4 | ASX | cXL3-13 |
| 165 | 19 | 59 | 53 | 8 | ASX | c34 |
| 166 | 13 | 57 | 76 | 8 | ASX | c34 |
| 167 | 9 | 42 | 96 | 8 | ASX | c34 |
| 168 | 16 | 62 | 85 | 8 | ASX | c34 |
| 169 | 8 | 47 | 90 | 3 | c34 | c34 |
| 170 | 9 | 49 | 93 | 3 | c34 | c34 |
| 171 | 13 | 50 | 80 | 5 | c34 | c34 |
| 172 | 7 | 40 | 96 | 3 | c34 | c34 |
| 173 | 4 | 40 | 99 | 4 | c34 | c34 |
| 174 | 4 | 43 | 98 | 4 | c34 | c34 |
| 175 | 31 | 45 | 86 | 2 | c34 | c34 |
| 176 | 18 | 48 | 80 | 2 | c34 | c34 |
| 177 | 35 | 52 | 67 | 4 | c34 | c34 |
| 178 | 18 | 43 | 85 | 2 | c34 | c34 |
| 179 | 16 | 79 | 93 | 3 | c34 | c34 |
| 180 | 17 | 58 | 94 | 3 | c34 | c34 |
| 181 | 46 | 60 | 87 | 7 | ASX | c34 |
| 182 | 39 | 67 | 74 | 7 | ASX | c34 |
| 183 | 38 | 65 | 82 | 7 | ASX | c34 |
| 184 | 30 | 61 | 73 | 7 | ASX | c34 |
| 185 | 30 | 56 | 66 | 6 | ASX | c34 |
| 186 | 38 | 67 | 66 | 7 | ASX | c34 |
| 187 | 27 | 56 | 72 | 6 | ASX | c34 |
| 188 | 31 | 63 | 87 | 7 | ASX | c34 |
| 189 | 44 | 76 | 71 | 6 | ASX | c34 |
| 190 | 32 | 57 | 69 | 7 | ASX | c34 |
| 191 | 21 | 57 | 80 | 7 | ASX | c34 |
| 192 | 27 | 55 | 70 | 6 | ASX | c34 |
| 193 | 16 | 55 | 68 | 10 +E | ASX | c34 |
| 194 | 16 | 51 | 87 | 9 + E | ASX | c34 |
| 195 | 12 | 56 | 82 | 5 + E | c34 | c34 |
| 196 | 7 | 54 | 97 | 3 + E | c34 | c34 |
| 197 | 7 | 54 | 97 | 3 + E | c34 | c34 |
| 198 | 9 | 53 | 95 | 3 + E | c34 | c34 |
| 199 | 28 | 50 | 93 | 9 + E | ASX | c34 |
| 200 | 24 | 52 | 99 | 8 + E | ASX | c34 |
| 201 | 25 | 58 | 82 | 4 + E | c34 | c34 |
| 202 | 13 | 59 | 87 | 2 + E | c34 | c34 |
| 203 | 18 | 62 | 89 | 2 + E | c34 | c34 |
| 204 | 11 | 53 | 84 | 2 + E | c34 | c34 |
| 205 | 27 | 55 | 86 | 8 + E | ASX | c34 |
| 206 | 20 | 50 | 98 | 7 + E | ASX | c34 |
| 207 | ND | ND | ND | 3 + E | c34 | c34 |
| 208 | ND | ND | ND | 1 + E | c34 | c34 |
| 209 | 14 | 58 | 66 | 1 + E | c34 | c34 |
| 210 | 15 | 70 | 61 | 1 + E | c34 | c34 |
| 211 | 42 | 58 | 96 | 9 | ASX | c34 |
| 212 | 33 | 50 | 99 | 8 | ASX | c34 |
| 213 | 29 | 49 | 99 | 4 | ASX | c34 |
| 214 | 27 | 51 | 97 | 5 | ASX | c34 |
| 215 | 20 | 48 | 77 | 6 | ASX | c34 |
| 216 | 24 | 49 | 97 | 6 | ASX | c34 |
| 217 | 15 | 43 | 99 | 4 | ASX | c34 |
| 218 | 13 | 51 | 96 | 5 | ASX | c34 |
| 219 | 21 | 50 | 99 | 5 | ASX | c34 |
| 220 | 18 | 50 | 99 | 6 | ASX | c34 |
| 221 | 23 | 51 | 98 | 7 | ASX | c34 |
| 222 | 29 | 60 | 96 | 6 | ASX | c34 |
| 223 | 19 | 62 | 98 | 7 + E | ASX | c34 |
| 224 | 15 | 76 | 92 | 2 + E | c34 | c34 |

### Antibody Binding to Human TL1A

Antibody binding to human TL1A (Fitzgerald #30R-AT070) was quantitated by ELISA. Briefly, a Corning Costar 3366 96-well round bottom high bind plate was coated with 50 μL TL1A (1 μg/mL) in PBS overnight at 4° C. The plate was washed 3× with PBS-0.05% Tween 20 (PBS-T) and was blocked with 100 μL 1% BSA/PBS for 1 h at 25° C. The block was removed, and culture supernatant diluted 5-fold was added and serially diluted 2-fold across the plate. Samples were incubated for 1 h at 25° C., the plate was washed three times with PBS-T, and 50 μL anti-Fc HRP secondary, diluted 1:4000 in BSA/PBS was added for 1 h at 25° C. The plate was washed three times with PBS-T and developed for up to 15 min following the addition of 50 μL Ultra TMB ELISA substrate. The reaction was terminated by the addition of 50 μL 2 N H₂SO₄ and the A450 nm was measured. The antibody affinities, as determined by ELISA titration against human TL1A using unpurified culture supernatants, is shown in Table 2.

### Purification of Antibodies

Antibodies were purified from culture supernatants in a single step using Dynabeads Protein A (ThermoFisher Scientific, cat. #10002D). First, culture supernatants were concentrated per manufacturer's instructions using an Amicon Ultra-4 Centrifugal Filter Unit (30,000 MWCO; MilliporeSigma, cat. #C7719). The Dynabeads were resuspended by gentle vortexing and 100 μL were transferred to an Eppendorf tube. Using a magnet to retain the beads, the storage buffer was removed, and the beads were washed with 0.5 mL of 20 mM sodium phosphate, 150 mM NaCl, pH 7.4 (EB, Equilibration Buffer). A total of up to 24 μg of IgG from culture supernatant was added to the beads and mixed gently until the beads were resuspended. When necessary, antibody supernatants were diluted with EB. The tubes were placed sideways on a shaking platform and mixed for 10 min at 25° C. at 500 rpm. Subsequently, the beads were collected at the bottom of the tube using a microfuge at 10,000 rpm for 30 sec. Using a magnet to retain the beads, the supernatant was removed. The beads were washed once with 0.5 mL of 20 mM sodium phosphate, 500 mM NaCl, pH 7.4 followed by another wash with 50 mM sodium phosphate, pH 6.0. The beads were collected at the bottom of the tube using a microfuge at 10,000 rpm for 30 sec. Purified antibody was eluted from the beads using 20 μL 50 mM sodium acetate, pH 3.5 with gentle mixing for 2 min at 25° C. Using a magnet to retain the beads, the eluate was transferred to a fresh tube containing 1.1 μL 1 M Tris, pH 8.5 to neutralize the pH of the sample. This sample was then centrifuged at 10,000 rpm for 2 min and transferred to a fresh tube to ensure removal of residual Dynabeads. The concentration of the purified sample was determined using a DeNovix DS-11 Spectrophotometer/Fluorometer, buffer blank, and a mass extinction coefficient of 13.70 at 280 nm for a 1% IgG solution.

### Size Exclusion Chromatography

The antibodies were analyzed by size exclusion chromatography (SEC) to determine percent monomer and identify any large molecular weight aggregate contaminant species. A total volume of 15 μL of protein A purified antibodies at a concentration of 0.1-1 μg/μL were analyzed using a Waters SEC column (Acquity UPLC BEH SEC, 200 Å, 1.7 μm, 4.6×150 mm) on a Shimadzu UPLC instrument at a flow rate of 0.2 mL/min and a column oven temperature of 30° C. Standard PBS was used as the mobile phase and absorbance at 280 nm was used to monitor protein elution. For some antibody clones tested that demonstrated non-symmetrical elution profiles, PBS buffer supplemented with 350 mM NaCl at pH 6.0 was utilized to reduced non-specific interactions with the column matrix. The percent main peak (monomer) value was calculated using the Shimadzu software. The monomeric content of purified antibody variants is shown in Table 2.

### Example 3: Design of Humanized Anti-TL1A Antibodies with Reduced Effector Function

In certain cases, it might be beneficial to reduce the potential effector function of the antibodies. Multiple strategies to diminish effector function have been described, including point mutations to ablate FcγR and C1q binding, cross-subclass Fc designs to eliminate FcγR and C1q binding, and glycoengineering to ablate FcγR and C1q binding. Representative examples are highlighted in Table 3.

TABLE 3
Representative approaches to abrogating effector function

| Mutation(s) | Effect |
|---|---|
| E233P | Decreases binding to FcγRI, II, III |
| S228P, L235E SPLE in IgG4 | Decreases binding to FcγRI |
| L235E | Decreases binding to FcγRs |
| L234A, L235A | Decreases binding to FcγRI, II, III |
| L234A, L235A, G237A | Decreases binding to FcγRI, II, III, C1q |
| L234A, L235A, P329G | Decreases binding to FcγRI, II, III, C1q |
| L234F, L235E, P331S | Decreases binding to FcγRI, II, III, C1q |
| L234A, L235E, G237A | Decreases binding to FcγRI, II, III, C1q |
| L234A, L235E, G237A, P331S | Decreases binding to FcγRI, II, III, C1q |
| L234A, L235A, G237A, P238S, | Decreases binding to FcγRI, IIa, IIb, IIIa |
| H268A, A330S, P331S (IgG1σ) | |
| L234A, L235A, P329A | Decreases binding to FcγRI, II, III, C1q |
| G236R, L328R | Decreases binding to FcγRI, II, III |
| G237A | Decreases binding to FcγRII |
| F241A | Decreases binding to C1q |
| V264A | Decreases binding to C1q |
| D265A | Decreases binding to FcγRI, II, III |
| D265A, N297A | Decreases binding to FcγRI, II, III, C1q |
| D265A, N297G | Decreases binding to FcγRI, II, III, C1q |
| D270A | Decreases binding to C1q |
| N297A, G, D, Q | Elimination of N-linked glycosylation |
| | Decreases binding to FcγRI, II, III, C1q |
| P329A, G, R | Decreases binding to C1q |
| A330L | Decreases binding to C1q |
| P331A, S | Diminished C1q binding |
| IgG2 | Decreases binding to FcγRs |
| IgG4 | Decreases binding to FcγRs; |
| | Does not activate complement system |
| S228P | Prevent IgG4 Fab arm exchange |
| S228P, F234A, L235A (IgG4) | Decreases binding to FcγRI, IIa, IIIa |
| IgG2-IgG4 cross-subclass | Decreases binding to FcγRI, II, III, C1q |
| (IgG2/G4) | |
| IgG2-IgG3 cross-subclass | Decreases binding to FcγRs; |
| | Decreases binding to C1q |
| H268Q, V309L, A330S, P331S | Decreases binding to FcγRI, II, III, C1q |
| (IgG2m4) | |
| V234A, G237A, P238S, H268A, | Decreases binding to FcγRI, IIa, IIb, |
| V309L, A330S, P331S (IgG2σ) | IIIa, C1q |
| High mannose glycosylation | Decreases binding to C1q |

In order to express antibodies with abrogated effector function, the light chain variable regions of the antibodies disclosed in Example 2 and Table 1 are cloned with a kappa light chain constant region, while the heavy chain variable regions are cloned with a modified IgG1 heavy chain backbone, or a modified IgG2 backbone, or a modified IgG4 backbone, or an unmodified IgG2 or IgG4 backbone, such as those disclosed in Table 3, Table 13, Table 9B, or elsewhere.

The impact of the various Fc engineering approaches on CDC activity can be assessed using C1q binding and C3 fixation assays. Purified antibodies are diluted in PBS and serial dilutions are plated on a microtiter plate for 12-18 h at 4° C. The plates are blocked with 5% gelatin/PBS containing 1% (v/v) Tween-20 for 1 h at 25° C. Subsequently, the plates are incubated with 10% (v/v) human sera in PBS and C1q binding is detected using 1:500 dilution of HRP-conjugated rabbit anti-C1q (Bioss Inc.) in PBS containing 1% (v/v) Tween-20. To test C3 fixation, a 1:1000 dilution of rabbit anti C3 (abcam) is used followed by a 1:2000 dilution of HRP-conjugated chicken anti-rabbit IgG (abcam). The plates are developed as described for antibody quantitation assays in Example 1. EC50 values are calculated by fitting the data to a log (agonist) vs. response-variable slope (four parameter) model using GraphPad Prism (Sunnyvale, CA).

Additionally, the variants may be characterized for the binding of isolated C1q. MaxiSorp 384-well plates (Thermo Scientific, Nunc) are coated with serially diluted antibodies in 50 mM carbonate buffer, pH 9.6 (coat buffer), for 12-18 h at 4° C. Plates are washed with phosphate buffered saline (PBS) containing 0.05% polysorbate 20, pH 7.4 and blocked with PBS containing 0.5% BSA, 0.05% polysorbate 20, 15 ppm Proclin and 10% Blocker Casein (ThermoScientific), pH 7.4. After 1-hour incubation at 25° C., plates are washed. Human C1q (Quidel, San Diego, CA) in the same buffer is added and incubated for 1.5 hour. Bound C1q is detected by adding 20 ng/mL biotinylated mouse anti-mouse C1q (Hycult biotech; cross reacting with human C1q) for 1.5 hour followed by horseradish peroxidase (HRP)-conjugated streptavidin (GE Healthcare Life Sciences) for 1 hour. To check for coating efficiency, some coated wells receive buffer only for the first two incubation steps and receive goat anti-human Fab′2-HRP when the wells used for measuring C1q binding received streptavidin-HRP. Plates are washed after each incubation step. Peroxidase activity is detected with substrate 3, 3′, 5, 5′-tetramethyl benzidine (TMB) (Kirkegaard & Perry Laboratories). The reaction is stopped with 1M phosphoric acid and absorbance is measured at 450 nm. Dose-response binding curves are fitted with a four-parameter model and EC50 values are calculated using GraphPad Prism (Sunnyvale, CA).

The impact of the various Fc engineering approaches on ADCC activity is assessed using soluble FcγR receptor binding ELISAs. Soluble human FcγRI, FcγRIIb and FcγRIII (binding affinity to both the F158 and V158 polymorphic forms of FcγRIII is assessed) are expressed as recombinant fusion proteins with Gly-His6-glutathione-S-transferase (GST) at the C-terminus of the extracellular domain of the receptor. MaxiSorp 384-well plates are coated with 1 μg/ml human FcγR in coat buffer. Plates are washed and blocked with PBS containing 0.5% BSA, 15 ppm Proclin, pH 7.4. After a 1 h incubation, plates are washed and 3-fold serial dilution of antibodies in PBS containing 0.5% BSA, 0.05% polysorbate 20, 15 ppm Proclin, pH 7.4 is added to the plates and incubated for 2 h. For enhanced binding sensitivity due to avidity, immune complexes are formed using anti-human antibody. Bound antibody is detected with HRP-conjugated goat anti-human kappa (Southern Biotech) using Ultra TMB substrate as described in Example 1. The reaction is terminated and the plate is read as described above. The dose-dependent binding curve of the wild type antibody (no Fc modifications) is fitted with GraphPad Prism (Sunnyvale, CA) four parameter curve fitting program. The relative affinity of the variant vs. the wild type is estimated by dividing the equivalent ng/ml wild type concentration at the appropriate concentration.

In addition, the variants are tested directly in Fc effector bioassays (Promega) following manufacturer's directions. These assays include FcγRIIa-H ADCP Bioassay (Promega cat #G9901), ADCC Reporter Bioassays, FcγRIIIa F Variant (Promega, cat #G9798), ADCC Reporter Bioassays, FcγRIIIa V Variant (Promega, cat. #G7015). The variants are tested both as monomeric Ig and as small immune complexes (ICs) by using an anti-hu Ig antibody to form small ICs.

A Europium based ADCC assay is performed. Briefly, peripheral blood lymphocytes (PBLs) are isolated by Ficoll Paque Plus gradient centrifugation. The PBLs are collected, washed with RPMI1640, 10% FCS and resuspended in cell culture medium. The cells are diluted to 2.5×10⁶ cells/ml. Target cells are labelled with BADTA (2,2′: 6′,2″-terpyridine-6,6″-dicarboxylic acid acetoxymethylester): Cells are harvested by adding Accutase (Millipore), washed once and diluted to 1×10⁶ cells/ml. Next, 2.5 μL BADTA is added per 1×10⁶ cells and incubated for 35 min at 37° C. with 5% CO₂. After labelling the cells are diluted with 10 ml culture medium, centrifuged at 200×g for 10 min and supernatant aspirated. This step is repeated 3× with culture medium/2 mM Probenicid and the sample is diluted to 1×10⁵ cells/ml, centrifuged at 300×g for 5 min, supernatant taken off and 50 μL pipetted into the wells intended for the background controls. The final ratio of effector (PBL) to target cells is 25:1.

Controls include: (1) Background: the 50 μL aliquot, diluted with 100 μL medium, (2) Spontaneous lysis: 50 μL of the labelled target cell suspension plus 100 μL culture medium, incubated 2 h at 37° C., (3) Maximal lysis: 50 μL/well of the labelled target cell suspension plus 100 μL Triton X-100 (0.5% in PBS) incubated 2 h at 37° C., (4) Lysis control without antibodies: 50 μL/well of the labelled target cell suspension and 50 μL culture medium plus 50 μL of effector cells incubated 2 h at 37° C., (5) Lysis control without effector cells: 50 μL/well of the labelled target cell suspension; add 50 μL culture medium plus antibody at highest concentration used and incubate 2 h at 37° C.

At the end of the incubation period the 96 well plate is centrifuged at 100 rpm. 20 μL of each supernatant is transferred into an OptiPlate HTRF-96 (Packard) and 200 μL Europium solution is added and incubated for 15 min on a shaker. Fluorescence is measured as for time resolved fluorescence and spontaneous release and specific release are calculated.

A CDC assay is performed. Briefly, target cells are washed and diluted to 1×10⁵ cells/ml and 100 μL/well (10+ cells) are added to a 96-well flat bottom microtiter plate. A titration curve of the test antibody is created using serial dilutions, beginning at 1 μg/mL. Antibody is added to the plate, mixed gently, and is then placed at 37° C./5% CO2 incubator for 30 min. Next, 25 μL freshly dissolved baby rabbit complement (Cedarlane CL3441, 1 ml lyophilized, dilute freshly in 4 ml double distilled water) is added, mixed gently, and the plate is incubated at 37° C./5% CO2 incubator for 30 min. After the incubation period 50 μL supernatant is taken off and 100 μL Cell Titer Glo. reagent (Promega Corp.) is added to the remaining 100 μL supernatant. The plate is placed on an orbital shaker for 2 min, 100 μL/well is transferred into a black luminescence microtiter plate (Costar) and luminescence is measured. Controls included: (1) medium control (target cells plus 50 μL medium), (2) maximal lysis control (target cells plus 50 μL 0.5% Triton X-100), (3) complement control (target cells plus 25 μL medium plus 25 μL complement).

As provided and described herein, Fc variants were designed to diminish effector function and subsequently tested for the ability to (i) effectively be purified/manufactured (Table 11), (ii) reduce antibody-dependent cell-mediated cytotoxicity (ADCC), and (iii) reduce complement-dependent cytotoxicity. Test articles tested comprise heavy chain SEQ ID NOs: 368-380. Heavy chains used were paired with a light chain comprising SEQ ID NO: 381. ELISA titration profiles and EC50s were generated against recombinant TL1A antigen (“EC50”, Table 12). Interestingly, Fc mutations did affect purity, as measured by monomer content, for select mutations/Fc variants (Table 11, wild-type IgG1 control).

### Reduction of CDC Activity

Test articles were evaluated for CDC activity, compared to negative control Human IgG4 isotype control, on TL1A-expressing HEK293 target cells. Rituxan (anti-CD20) was used as a positive technical control on CD20-expressing Raji cell. All test articles were used at a final top concentration of 10 μg/mL followed by a five-fold dilution series (7 points total), in addition to a no treatment control, in triplicate. Cells were incubated with test articles for 15 minutes at 37 C, then treated with human complement, at a final concentration of 25%, for 3 hours at 37 C, 5% CO2. Following incubation, cells were washed and resuspended in Propidium Iodide (P.I.) at a final concentration of 5 μg/mL prior to flow cytometry analysis. Total cells were examined by flow cytometry during sample acquisition. Data were plotted on an XY chart, graphing percentage P.I. positive cells against the log of the concentration and fit to a non-linear regression curve. Cell cytotoxicity in the presence of all test articles was not distinguishable from cell cytotoxicity in the presence of isotype control (Table 12). CDC bioactivity was observed on Raji target cells with Rituxan treatment.

TABLE 11
anti-TL1A antibodies tested for reduced effector function

| | | | | | Purity | |
|---|---|---|---|---|---|---|
| | Fc SEQ | Heavy Chain | | | SDS- | SEC- |
| Class | ID NO | SEQ ID NO | mg/mL | mg | PAGE | HPLC |
| IgG1, | 326 | 368 | 2.65 | 10.60 | 95% | 90% |
| protein | 323 | 369 | 1.15 | 12.65 | 95% | 92% |
| variants | 329 | 370 | 3.22 | 10.62 | 90% | 89% |
| | 338 | 371 | 1.61 | 11.27 | 95% | 92% |
| | 344 | 372 | 3.43 | 10.29 | 95% | 91% |
| | 332 | 373 | 1.51 | 15.10 | 95% | 93% |
| | 363 | 374 | 2.85 | 11.40 | 95% | 92% |
| | 364 | 375 | 1.55 | 10.85 | 95% | 92% |
| IgG1, | 356 | 376 | 2.33 | 9.32 | 90% | 90% |
| glycan | 350 | 377 | 1.36 | 12.24 | 95% | 92% |
| knock- | | | | | | |
| out | | | | | | |
| IgG4 | 365 | 378 | 1.78 | 19.58 | 95% | 82% |
| | 366 | 379 | 2.33 | 18.64 | 90% | 81% |
| | 367 | 380 | 5.08 | 15.24 | 95% | 90% |
| Control | — | — | 3.70 | 5.55 | 95% | 97% |

TABLE 12
Effector function of anti-TLIA antibodies

| | Fc SEQ | Heavy Chain | EC50 | | |
|---|---|---|---|---|---|
| Class | ID NO | SEQ ID NO | (nM) | ADCC | CDC |
| IgG1, protein | 326 | 368 | 0.222 | ND | ND |
| variants | 323 | 369 | 0.215 | 100 ng/mL | ND |
| | 329 | 370 | 0.188 | ND | ND |
| | 338 | 371 | 0.220 | 10 μg/mL | ND |
| | 344 | 372 | 0.346 | ND | ND |
| | 332 | 373 | 0.347 | ND | ND |
| | 363 | 374 | 0.329 | ND | ND |
| | 364 | 375 | 0.330 | ND | ND |
| IgG1, glycan | 356 | 376 | 0.340 | ND | ND |
| knock-out | 350 | 377 | 0.293 | ND | ND |
| IgG4 | 365 | 378 | 0.299 | ND | ND |
| | 366 | 379 | 0.324 | ND | ND |
| | 367 | 380 | 0.252 | ND | ND |

### Reduction of CDC Activity

An antibody-dependent cell-mediated cytotoxicity (ADCC) reporter assay was performed for the characterization of test articles and IgG4 Isotype control on HEK 293 TL1A cells. A reporter cell line engineered to express human Fc-gamma-RIIIa V158 (high affinity) served as effector cells.

Test articles were evaluated with a top concentration of 10 μg/mL (log dilution for 7 points total, in addition to no test article control). Treatment conditions were tested in triplicate, effector and target cells were co-cultured for 6 hours at 37 C with 5% CO2. Raji target cells were used as a positive control, with Rituxan treatment at a top concentration of 10 μg/mL, 7-point log dilution series, and no treatment control. Test article 502 treatment resulted in dose-dependent increase in luciferase reporter gene activity, and 5044 treatment resulted in increase of reporter activity at the highest tested concentration. The rest of the test articles did not induce reporter activity (Table 12).

### Example 4: Characterization of Potency and Species Selectivity in Whole Blood Assay

The relative potency of a panel of candidate antibodies was first assessed by determining the inhibition of interferon gamma release in human blood using the antibodies at 1 and 10 nM. All of the antibodies displayed potent activity, with A219 appearing to be one of the most potent candidates (Table 4).

TABLE 4
Inhibition of interferon gamma release in human
blood with anti-TL1A

| Clone | % Inhibition at 1 nM Ig | % Inhibition at 10 nM Ig |
|---|---|---|
| A147 | 51.3 | 72.4 |
| A212 | 46.8 | 71.2 |
| A213 | 48.6 | 69.8 |
| A217 | 46.0 | 72.2 |
| A219 | 59.8 | 75.2 |
| A220 | 36.9 | 63.2 |

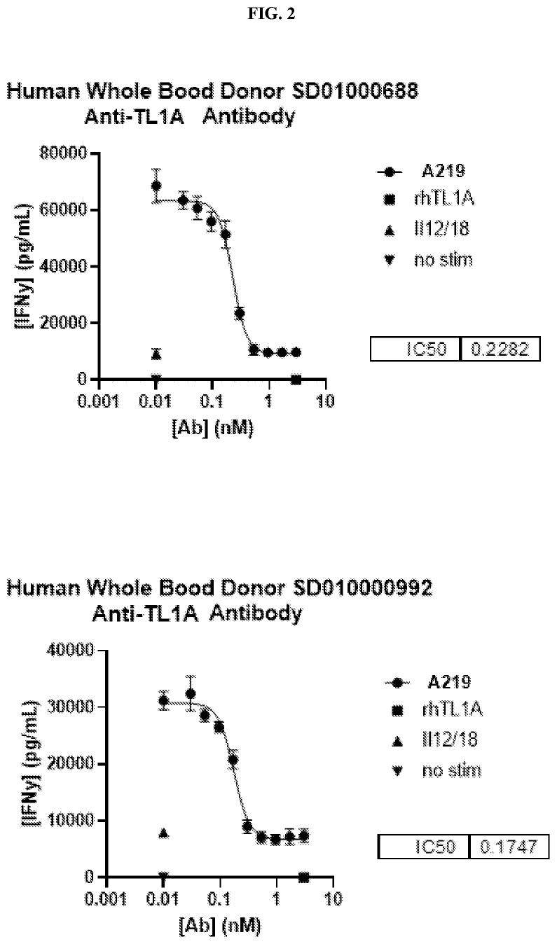

Next, three of the variants were characterized for inhibition of interferon gamma release in human blood using multiple human blood donors and testing the antibodies across a broader range of concentrations (0.01-100 nM). Representative inhibition profiles of A219 are shown in FIG. 2. The mean IC50 values for the variants, and a control antibody termed 1D1, for the inhibition of interferon gamma release from multiple human donors is shown in Table 5.

TABLE 5
IC50 values

| Clone | Mean | SD |
|---|---|---|
| A212 | 51.3 | 72.4 |
| A213 | 46.8 | 71.2 |
| A219 | 48.6 | 69.8 |
| 1D1 | 46.0 | 72.2 |

### Example 5: Properties of an Anti-TL1A Antibody

Physical and chemical properties of the anti-TL1A antibody A219 are shown in

TABLE 18
Physical and chemical properties of A219

| Property | Description |
|---|---|
| Molecular Weight¹ | 143,938 Daltons |
| Antibody class/subclass | IgG1 |
| Glycosylation | A219 has an N-linked carbohydrate attachment at a single site, Asn296 of |
| | the heavy chain. The major glycosylation species is GOF |
| TL1A binding affinity (K_{D})² | Human TL1A 59.0 +/− 15.6 pM |
| | Cynomolgus TL1A 36.3 +/− 12.8 pM |
| | Murine TL1A No significant binding |
| Specificity | No binding to the closely related human cytokines TNFSF6 (FasL), TNFSF10 |
| | (TRAIL) and TNFSF14 (LIGHT) tested as recombinant proteins |
| Extinction coefficient³ | 1.410 mg⁻¹ · mL · cm⁻¹ |
| pI | 8.8 |

¹Unglycosylated calculated molecular weight from the amino-acid sequence
²Three independent measurements, +/−standard deviation
³Calculated extinction coefficient from the amino-acid sequence

### Example 6: Animal Model of Colitis

The efficacy of anti-TL1A antibodies in animal models of colitis is performed. Anti-TL1A antibodies are tested in rodent models of acute colitis induced by intrarectal administration of di- or tri-nitrobenzenesulfonic acid (D/TNBS) or oxazolone, and chronic colitis induced by administration of DSS in drinking water or transfer of CD45RB^{hi} T cells. DNBS and oxazolone induce localized ulceration and inflammation. DSS administration induces robust generalized inflammation of the intestinal tract characterized by erosive lesions and inflammatory infiltrate. Symptoms of all these models usually include diarrhea, occult blood, weight loss and occasionally rectal prolapse. In a prophylactic model, antibody treatment begins at the start of administration of the colitis-inducing compound. In a therapeutic model, antibody treatment begins several days after commencement of induction. The effect of the treatment on weight, stool consistency and occult blood, as well as microscopic effects on epithelial integrity and degree of inflammatory infiltrate is determined. Daily clinical scoring is performed based on stool consistency and presence of occult blood giving a disease activity index (DAI) score.

### Example 7: Summary of Pharmacology, Pharmacokinetic, and Toxicology Studies

The anti-TL1A antibody A219 binds human tumor necrosis factor-like cytokine 1A (TL1A) with high affinity and specificity and neutralizes TL1A functional activity in in vitro and ex vivo cell-based assays. A219 binds to both human and cynomolgus TL1A with similar affinity (K D values of 0.06 nM and 0.04 nM, respectively). In addition, A219 is specific for TL1A and does not bind to other tumor necrosis factor super family (TNFSF) members. A219 blocks human TL1A-induced caspase activation in the TF-1 functional assay with an IC50 of 0.27 nM. A219 inhibits TL1A-mediated interferon gamma release from peripheral blood mononuclear cells (PBMCs) in whole blood from monkeys administered doses of ≥0.056 mg/kg. In addition, a dose-dependent increase in circulating soluble (sTL1A) concentrations was observed at all dose levels in these monkeys. This suggests that systemic sTL1A levels may be a useful PD marker for target engagement by A219.

The nonclinical pharmacokinetics (PK) of A219 were characterized in the monkey and support the proposed once every other week dosing regimen in humans. The nonclinical PK of A219 is as expected for a monoclonal antibody that exhibits target-mediated drug disposition (TMDD) at lower doses and linear PK at higher dose levels that saturate the target-mediated route of clearance.

A219 was administered to monkeys once weekly via IV injection for up to 6 weeks (7 total doses). Most, if not all, of the findings observed after IV administration of A219 to monkeys in the 6-week repeat-dose toxicity study were considered to be secondary to generation of ADA in response to administration of a foreign protein (humanized monoclonal antibody) to immunocompetent animals. Based on electrocardiograms (ECGs), daily and detailed weekly clinical observations, and microscopic evaluation of the relevant tissues/organs there were no cardiovascular, central nervous system (CNS) or respiratory system effects observed in monkeys during 6 weeks of once weekly IV administration of A219 at up to 300 mg/kg/week. The clinically relevant no observed adverse effect level (NOAEL) in this study was considered to be 300 mg/kg/week (the highest dose tested). There was no off-target binding of A219 noted in the tissue cross-reactivity study with human or monkey tissues. There was no A219-related cytokine release in the human PBMC or whole blood cytokine release assays, nor in monkeys during the 6-week repeat-dose toxicity study. A219 did not cause complement-dependent cytotoxicity (CDC) or antibody-dependent cellular cytotoxicity (ADCC) of target expressing cells in Fc effector function assays.

### Example 8: Biophysical Properties of Anti-TL1A Antibodies at High Concentrations

The data for A219 anti-TL1A antibody properties in solution were analyzed together using a chemometric method termed partial least squares (PLS). Detailed descriptions of PLS modeling have been published in, for example, Katz, M. H. Multivariate Analysis: A Practice Guide for Clinicians. Cambridge University Press, New York, pp. 158-162 (1999); Stahle, L., Wold, K., Multivariate data analysis and experimental design in biomedical research. Prog. Med. Chem. 1988, 25:291-338; Wold S. PLS-regression: a basic tool of chemometrics. Chemom. Intell. Lab. Syst. 2001, 58:109-130; and Martens, H.; Martens, M. Multivariate Analysis of Quality: An Introduction, Wiley and Sons, Chichester, UK (2001). The calibration sets (blue lines) use all data for the model, while validation sets (red lines) leave out one sample at a time and rebuild the model for an assessment of ruggedness.

The viscosity was measured using an m-VROC™ viscometer by Rheosense with an A10 chip. The shear rates employed were about 1820 s−1. The viscometer was temperature controlled using a ThermoCube thermoelectric chiller and the samples were delivered using a Hamilton 100 μL syringe (81060). The accuracy of the instrument was verified using neat Isopropyl alcohol and measured at 25° C. Furthermore, across the concentration range tested, the percent increase in the HMW fraction as measured by size exclusion chromatography ranged from 0% to a 1.3% increase. HMW as used herein refers to high molecule weight antibody fraction, e.g., aggregated protein, and which excludes monomeric antibody.

Table 25 and Table 26 provide example formulations evaluated.

TABLE 25
Round 1 formulations

| Form No | protein | pH | phos | His | citrate | acetate | NaCl | sorbitol |
|---|---|---|---|---|---|---|---|---|
| 1 | 130 | 6.5 | 20 | 0 | 0 | 0 | 0 | 270 |
| 2 | 130 | 6.5 | 20 | 0 | 0 | 0 | 130 | 0 |
| 3 | 130 | 6.5 | 0 | 20 | 0 | 0 | 0 | 270 |
| 4 | 130 | 6.5 | 0 | 0 | 20 | 0 | 0 | 270 |
| 5 | 130 | 6.0 | 0 | 10 | 0 | 0 | 50 | 180 |
| 6 | 130 | 5.5 | 0 | 20 | 0 | 0 | 130 | 0 |
| 7 | 130 | 6.0 | 0 | 0 | 20 | 0 | 50 | 180 |
| 8 | 130 | 6.0 | 0 | 0 | 10 | 0 | 0 | 270 |
| 9 | 130 | 7.5 | 20 | 0 | 0 | 0 | 50 | 180 |
| 10 | 130 | 7.0 | 0 | 0 | 0 | 0 | 0 | 270 |
| 11 | 130 | 5.5 | 0 | 0 | 0 | 0 | 50 | 180 |
| 12 | 130 | 7.5 | 20 | 0 | 0 | 0 | 130 | 0 |
| 13 | 130 | 5.0 | 0 | 10 | 0 | 0 | 0 | 270 |
| 14 | 130 | 5.0 | 0 | 0 | 0 | 20 | 50 | 180 |
| 15 | 130 | 5.0 | 0 | 0 | 0 | 20 | 130 | 0 |
| 16 | 130 | 4.5 | 0 | 0 | 0 | 10 | 0 | 270 |

TABLE 26
Round 2 formulations

| Form | | | | | | | | | | | HP-b- |
|---|---|---|---|---|---|---|---|---|---|---|---|
| No | protein | pH | acetate | His | sucrose | sorbitol | NaCl | Gly | ArgHCl | LysHCl | CD |
| 1 | 130 | 5.0 | 20 | 0 | 0 | 0 | 130 | 0 | 0 | 0 | 0 |
| 2 | 130 | 5.0 | 20 | 0 | 0 | 180 | 50 | 0 | 0 | 0 | 0 |
| 3 | 170 | 5.5 | 0 | 20 | 0 | 0 | 0 | 270 | 0 | 0 | 0 |
| 4 | 170 | 5.0 | 10 | 0 | 0 | 180 | 0 | 0 | 50 | 0 | 0 |
| 5 | 150 | 5.0 | 0 | 0 | 270 | 0 | 0 | 0 | 0 | 0 | 0 |
| 6 | 150 | 5.5 | 10 | 0 | 220 | 0 | 0 | 0 | 0 | 0 | 75 |
| 7 | 170 | 4.5 | 10 | 0 | 0 | 0 | 0 | 270 | 0 | 0 | 0 |
| 8 | 170 | 5.5 | 0 | 0 | 0 | 0 | 100 | 0 | 0 | 0 | 50 |
| 9 | 150 | 5.0 | 0 | 20 | 0 | 220 | 0 | 0 | 25 | 0 | 0 |
| 10 | 150 | 5.5 | 0 | 10 | 150 | 0 | 50 | 0 | 0 | 0 | 0 |
| 11 | 170 | 5.5 | 0 | 0 | 0 | 0 | 0 | 120 | 0 | 50 | 0 |
| 12 | 130 | 6.0 | 0 | 20 | 220 | 0 | 0 | 0 | 0 | 25 | 0 |
| 13 | 170 | 5.0 | 0 | 20 | 0 | 0 | 50 | 0 | 0 | 0 | 100 |
| 14 | 170 | 5.5 | 20 | 0 | 0 | 180 | 0 | 0 | 50 | 0 | 0 |

### Viscosity

FIG. 3A depicts the comparison between the predicted and measured viscosity, where viscosity is in units of mPa-s. FIGS. 3B-3D demonstrates viscosity as a function of antibody concentration and pH. Antibody concentration ranged from greater than about 125 mg/mL to greater than about 170 mg/mL. pH ranged from less than 5.0 to about 7.5. Concentration dependence is evident, with very low viscosities (e.g., as indicated by a viscosity less than 5 mPa-s or 7 mPa-s). All formulations show low viscosities (<10 mPa-s), even at 170 mg/mL. FIG. 3E depicts the effects of pH versus acetate concentration at an antibody concentration of 150 mg/mL on viscosity. There is a slight pH dependence, with minimal viscosity near pH 6. FIG. 3F shows the effect of sucrose versus NaCl on viscosity at a pH pf 5.5 and an antibody concentration of 150 mg/mL. NaCl helps reduce viscosity, while HP-b-CD significantly increases viscosity. FIG. 3G depicts the effect of ArgHCl versus LysHCl at a pH of 5.5. ArgHCl increases viscosity slightly, while LysHCl has small effect. The formulated anti-TL1A antibodies also exhibited low viscosity (less than 16 mPa-s) at 200 mg/ml anti-TL1A.

### Aggregation

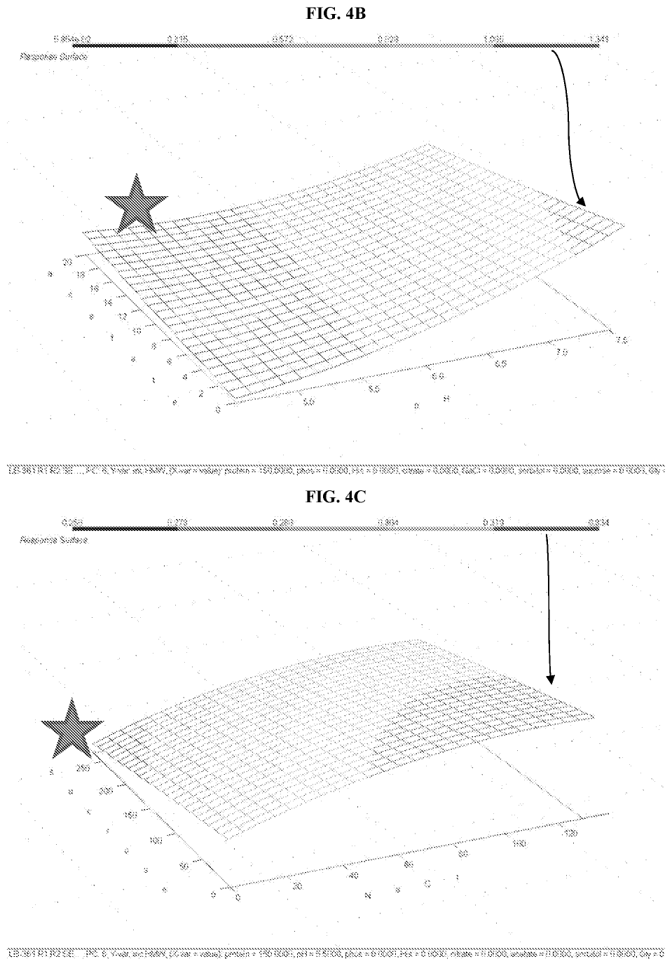

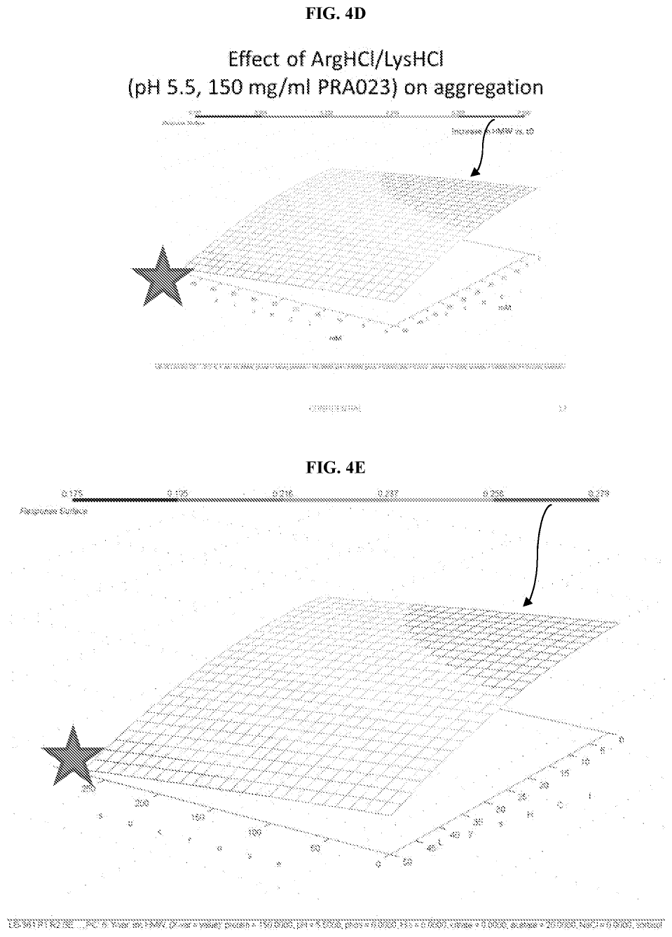

FIG. 4A depicts the PLS1 model for the effect on high molecular weight (HMW) aggregates at 2C and 25° C. FIGS. 4B-4E depict the effect of different parameters on aggregation. The response surface shows the increase in HMW over time. FIG. 4B depicts the effect of pH versus acetate on aggregation at an antibody concentration of 150 mg/mL. A lower pH leads to less aggregation (by SEC), using the PLS12 model, including all formulations with an increase in HMW species (%) by SEC as the endpoint. FIG. 4C depicts the effect of sucrose versus NaCl concentration on aggregation at a pH of 5.5 and an antibody concentration of 150 mg/mL. FIG. 4D depicts the effect of ArgHCl versus LysHCl on aggregation at a pH of 5.5 and an antibody concentration of 150 mg/mL. FIG. 4E depicts the effect of sucrose concentration versus LysHCl concentration over time at a pH of 5.5 and an antibody concentration of 150 mg/mL with 20 mM acetate. Sucrose, sorbitol, and Lys reduce aggregation. The formulated anti-TL1A antibodies also exhibited low aggregation at 200 mg/ml anti-TL1A.

### Loss of Major Peak by Cation Exchange Chromatography CEX

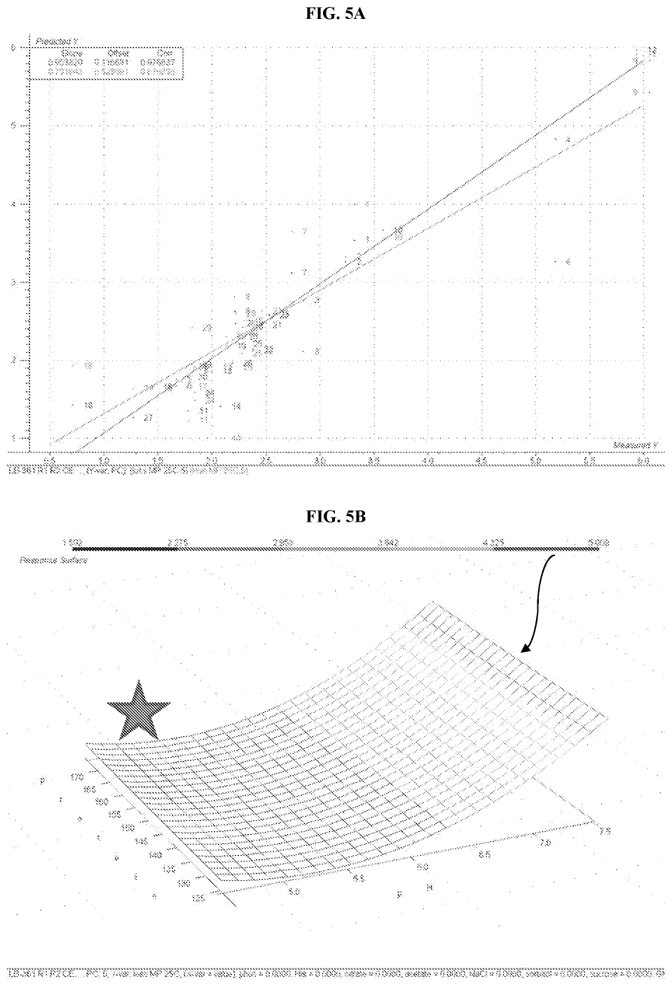

FIG. 5A depicts the predicted versus measured loss of main peak at 2 weeks and 25° C. FIGS. 5B-5E depict the effect of different parameters on the loss of main peak. The response surface indicates the percent loss of the main peak. FIG. 5B depicts the effect of pH and protein concentration on the loss of main peak in the CEX profile. The optimum pH for reducing loss of main peak by CEX is between 5 and 6. FIG. 5C depicts the effect of pH and acetate concentration on the loss of main peak in the CEX profile, at an antibody concentration of 150 mg/mL. FIG. 5D depicts the effect of sucrose and NaCl concentration on the loss of main peak in the CEX profile, at an antibody concentration of 150 mg/mL and a pH of 5.5. FIG. 5E depicts the effect of LysHCl and sucrose concentration on the loss of main peak in the CEX profile, at an antibody concentration of 150 mg/mL, pH of 5.5, with 20 mM concentration of acetate. The formulated anti-TL1A antibodies also exhibited low levels of loss of main peak at 200 mg/ml anti-TL1A.

### Example 9: The Effects of Polysorbate 20 or Polysorbate 80 on Storage Stability

After two rounds of formulation screening based on storage stability at different temperatures, Round 3 was designed to evaluate the interfacial sensitivity of two different base formulations in the presence (and absence) of varying amounts of polysorbate: PS20 and PS80. Repeated freeze-thaw (F/T) stress and agitation (Ag) were used as stress conditions. Two base formulations of anti-TL1A A219 at ˜200 mg/ml were evaluated, as seen in Table 15.

TABLE 15
Formulation design

| | Form | | acetate | protein | Sucrose | NaCl | LysHCl | PS20 | PS80 |
|---|---|---|---|---|---|---|---|---|---|
| Form | No | pH | (mM) | (mg/ml) | (mM) | (mM) | (mM) | (%) | (%) |
| 1 | 1 | 5.3 | 20 | 200 | 240 | 0 | 25 | 0 | 0 |
| 1-A | 1 | 5.3 | 20 | 200 | 240 | 0 | 25 | 0.01 | 0 |
| 1-B | 1 | 5.3 | 20 | 200 | 240 | 0 | 25 | 0 | 0.01 |
| 1-C | 1 | 5.3 | 20 | 200 | 240 | 0 | 25 | 0.02 | 0 |
| 1-D | 1 | 5.3 | 20 | 200 | 240 | 0 | 25 | 0 | 0.02 |
| 1-E | 1 | 5.3 | 20 | 200 | 240 | 0 | 25 | 0.05 | 0 |
| 1-F | 1 | 5.3 | 20 | 200 | 240 | 0 | 25 | 0 | 0.05 |
| 2 | 2 | 5.3 | 20 | 200 | 0 | 140 | 0 | 0 | 0 |
| 2-A | 2 | 5.3 | 20 | 200 | 0 | 140 | 0 | 0.01 | 0 |
| 2-B | 2 | 5.3 | 20 | 200 | 0 | 140 | 0 | 0 | 0.01 |
| 2-C | 2 | 5.3 | 20 | 200 | 0 | 140 | 0 | 0.02 | 0 |
| 2-D | 2 | 5.3 | 20 | 200 | 0 | 140 | 0 | 0 | 0.02 |
| 2-E | 2 | 5.3 | 20 | 200 | 0 | 140 | 0 | 0.05 | 0 |
| 2-F | 2 | 5.3 | 20 | 200 | 0 | 140 | 0 | 0 | 0.05 |

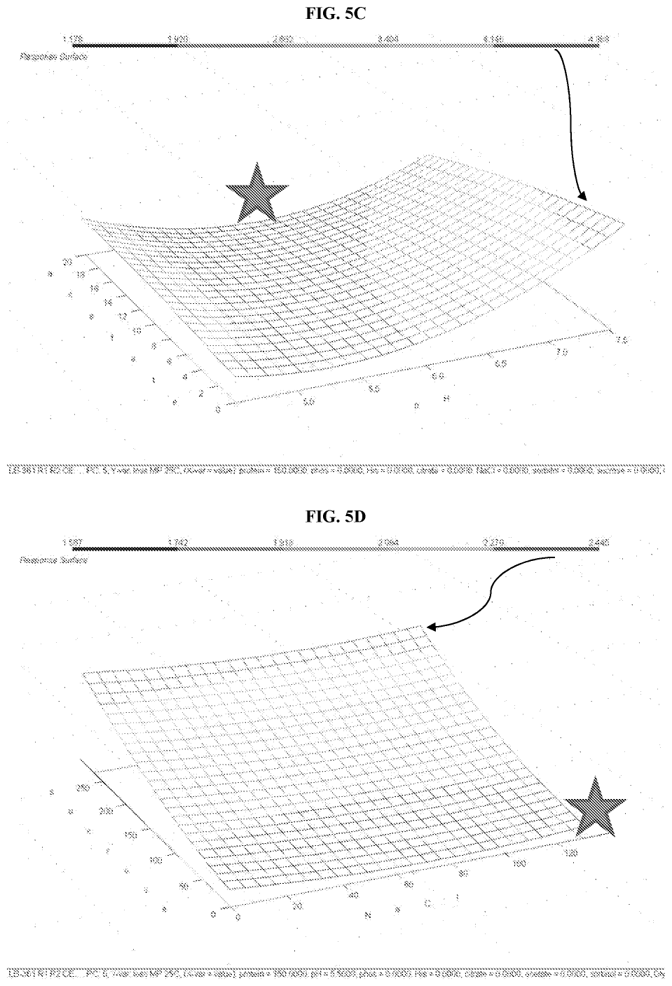

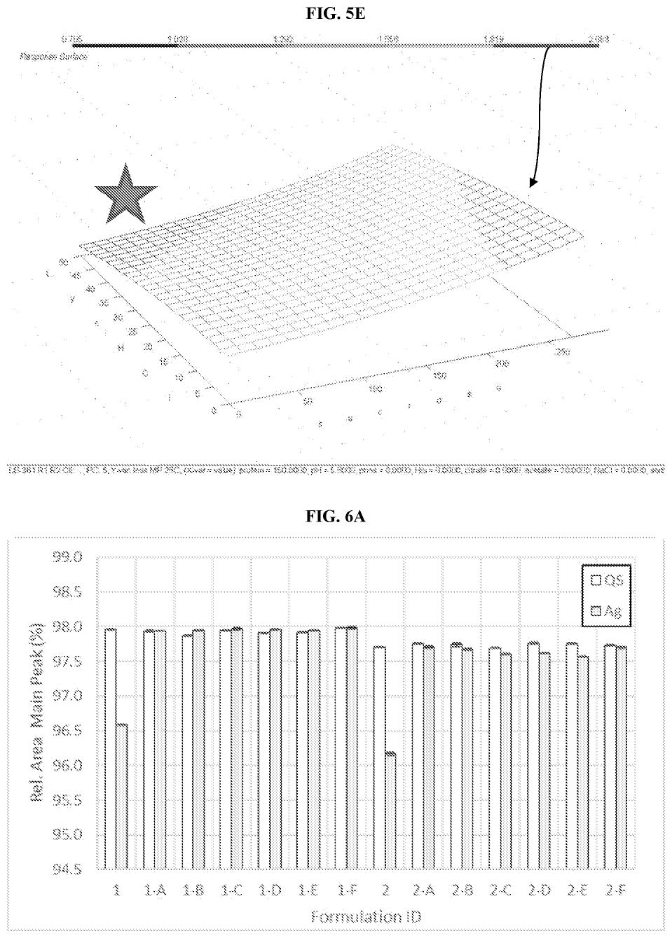

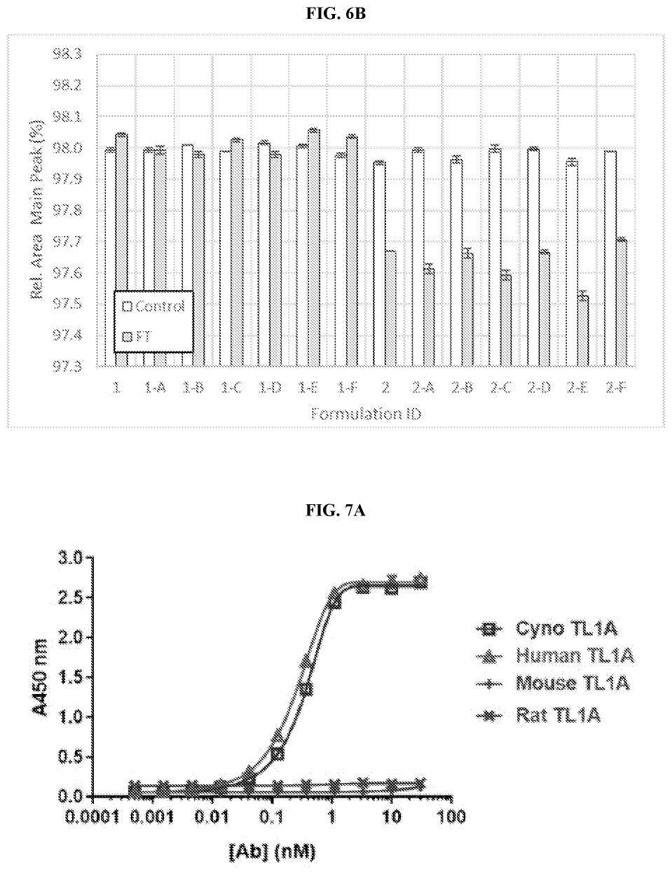

Results are depicted in Tables 16-17 and FIGS. 6A-6B. FIG. 6A depicts the loss of monomer by size exchange chromatography (SEC) with agitation. FIG. 6B depicts the loss of monomer by SEC with freeze-thaw. The results demonstrate that both PS 20 and PS 80 surfactants provide a stabilization benefit. There was very weak concentration dependence observed for both surfactants. Additionally, there was no appreciable chemical damage during short-term stress seen by CEX.

TABLE 16
Visual Appearance

| Form | QS | (Ag) | Control | FT |
|---|---|---|---|---|
| 1 | Clear | Cloudy | Clear | Clear |
| 1-A | Clear | Clear | Clear | Clear |
| 1-B | Clear | Clear | Clear | Clear |
| 1-C | Clear | Clear | Clear | Clear |
| 1-D | Clear | Clear | Clear | Clear |
| 1-E | Clear | Clear | Clear | Clear |
| 1-F | Clear | Clear | Clear | Clear |
| 2 | Clear | Cloudy | Clear | Clear |
| 2-A | Clear | Clear | Clear | Clear |
| 2-B | Clear | Clear | Clear | Clear |
| 2-C | Clear | Clear | Clear | Clear |
| 2-D | Clear | Clear | Clear | Clear |
| 2-E | Clear | Clear | Clear | Clear |
| 2-F | Clear | Clear | Clear | Clear |

TABLE 17
SEC results

| | QS samples | | | Agitation Samples | | |
|---|---|---|---|---|---|---|
| | HMW | Main Peak | LMW | HMW | Main Peak | LMW |
| | Rel. | Rel. | Rel. | Rel. | Rel. | Rel. |
| | Area | Area | Area | Area | Area | Area |
| Form | (%) | (%) | (%) | (%) | (%) | (%) |
| 1 | 1.99 | 97.97 | 0.05 | 3.37 | 96.59 | 0.04 |
| 1-A | 2.01 | 97.94 | 0.05 | 1.99 | 97.94 | 0.07 |
| 1-B | 2.08 | 97.87 | 0.05 | 2.00 | 97.94 | 0.06 |
| 1-C | 2.01 | 97.94 | 0.05 | 1.97 | 97.97 | 0.06 |
| 1-D | 2.04 | 97.92 | 0.05 | 1.97 | 97.97 | 0.07 |
| 1-E | 2.03 | 97.93 | 0.05 | 1.98 | 97.95 | 0.08 |
| 1-F | 1.97 | 97.99 | 0.04 | 1.94 | 97.99 | 0.07 |
| 2 | 2.24 | 97.71 | 0.05 | 3.80 | 96.17 | 0.03 |
| 2-A | 2.21 | 97.75 | 0.04 | 2.22 | 97.71 | 0.07 |
| 2-B | 2.21 | 97.74 | 0.05 | 2.25 | 97.68 | 0.07 |
| 2-C | 2.25 | 97.70 | 0.05 | 2.32 | 97.60 | 0.08 |
| 2-D | 2.19 | 97.76 | 0.05 | 2.31 | 97.62 | 0.08 |
| 2-E | 2.20 | 97.75 | 0.05 | 2.35 | 97.57 | 0.09 |
| 2-F | 2.21 | 97.74 | 0.05 | 2.23 | 97.70 | 0.07 |

### Example 10: Long Term Stability

Formulation 1 (150, 175, or 200 mg/ml of anti-TL1A; 20 mM acetate; pH 5.3; 240 mM sucrose; 25 mM LysHCl; 0.02% PS 20) and Formulation 2 (150, 175, or 200 mg/ml of anti-TL1A; 20 mM acetate; pH 5.3; 220 mM sucrose; 40 mM NaCl; 0.02% PS 20) are tested for long-term stability over 6 months. One set of formulations is stored at 5° C. and one set of formulations is stored at 25° C. pH, osmolality, protein concentration, and viscosity are measured at the beginning of the study and after 6 months. SEC, CEX, FlowCAM and visual appearance are used to monitor the stability at the beginning of the study and at 1 month, 2 months, 3 months, and 6 months into the study.

### Example 11: Pharmaceutical Properties and Formulation

Formulations of anti-TL1A A219 were prepared. An A219 formulation is a clear to slightly opalescent, colorless to slightly yellow liquid that is essentially free of foreign matter, supplied as 8.4 mL of a 60 mg/mL solution in a 10 mL SCHOTT Fiolax Type I Tubular Glass Vial sealed with a West Bromobutyl Rubber Stopper and West Flip-Off.

The qualitative composition of A219 is provided in Table 19 below.

TABLE 19
Example composition of anti-TL1A

| Ingredient | Quality Standard | Function |
|---|---|---|
| A219 Drug Substance | cGMP | Active Ingredient |
| Sodium Phosphate, mono basic monohydrate | USP, FCC, endotoxin tested | Buffer |
| Sodium Phosphate, di basic Heptahydrate | USP, FCC, endotoxin tested | Buffer |
| Sucrose | USP/NF, EP, JP | Stabilizer |
| Glycine | BP, EP, JP, USP | Stabilizer |
| Polysorbate-20 | NF, multi-compendial | Surfactant |
| Water for Injection (WFI) | USP/NF | Diluent |

BP = British Pharmacopoeia; cGMP = current Good Manufacturing Practice; EP = European Pharmacopeia; FCC = food chemicals codex; NF = National Formulary; JP = Japanese Pharmacopeia; USP = United States Pharmacopeia.

### Drug Product Preparation

Solutions of A219 may foam. Therefore, shaking or excessive agitation of vials is avoided. Additionally, care is taken to ensure the sterility of the prepared solution, as the drug product may not contain antimicrobial preservatives or bacteriostatic agents. A sufficient excess of drug product may be included in each single use vial to account for withdrawal losses.

Dilution of A219 injection is performed using sterile disposable latex-free syringes. An 18 gauge, 1.5 inch sterile needle is used for withdrawal from the vial. Prior to IV administration, A219 injection is diluted in a polyvinyl chloride (PVC) IV bag containing 0.9% Sodium Chloride Injection (normal saline [NS]), using aseptic technique, to prepare a dosing solution with a A219 concentrations between 0.01 and 8 mg/mL. The product is infused at the protocol-specific dose(s) and rate(s) through a PVC IV solution infusion set with a sterile, nonpyrogenic 0.2 μm polyethersulfone in line filter. It is not administered as IV push or bolus injection.

### Storage Conditions and Use Conditions

A219 formulated at 500 mg/vial (60 mg/mL) is stored in a refrigerator at a temperature of 2°-8° C. (38°-46° F.).

### Example 12: A219 Binding Selectivity

The predicted TL1A protein sequence in human was compared to the mouse, rat and cynomolgus monkey sequences. Mouse, rat and monkey protein sequences were 64%, 66%, and 98% homologous to human TL1A, respectively.

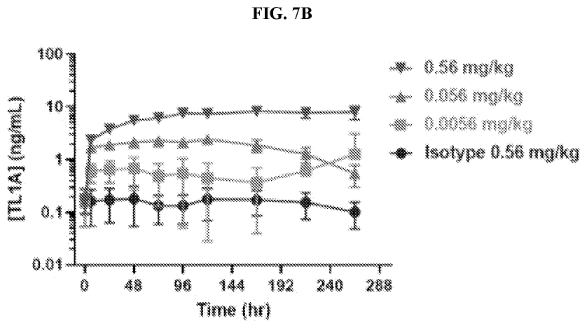

The binding of A219 to mouse, rat, cynomolgus monkey, and human recombinant TL1A protein was assessed in an ELISA. As shown in FIG. 7A, A219 binds to human and monkey TL1A with sub-nanomolar IC50 values of 0.33 nM and 0.47 nM, respectively. In contrast, A219 did not bind to mouse or rat TL1A protein.

A219 binding affinity and kinetics for recombinant human and monkey TL1A protein was assessed using surface plasmon resonance (SPR). A219 binds to human and cynomolgus TL1A with K_{D} values of 0.06 nM and 0.04 nM, respectively.

The binding of A219 to membrane-bound TL1A was assessed using human embryonic kidney 293 cells stably transfected with human TL1A (TNFSF15/HEK293 cells). A219 binds to membrane-bound TL1A expressed on the surface of TNFSF15/HEK293 cells in a dose-dependent manner with an EC50 value of 17.4 nM. There was no binding to the parental HEK293 cells.

TL1A is the only known ligand for its functional receptor DR3. TL1A is also capable of binding to Decoy receptor 3 (DcR3), a soluble TNF receptor without a transmembrane domain. The binding of A219 to other known ligands of DcR3, including TNFSF6 (FasL), TNFSF10 (TRAIL) and TNFSF14 (LIGHT) were assessed by ELISA. A219 did not bind to these TNF family members when tested at concentrations nearly 1,000-fold above the EC50 of the respective positive control antibodies.

### Example 13: In Vitro Functional Activity of Anti-TL1A

The ability of A219 to prevent DR3-mediated caspase activation by either human or monkey TL1A was assessed in cycloheximide-treated TF-1 cells. TF-1 cells are human erythroleukemic cells that natively express DR3, the functional receptor for TL1A. Human and cynomolgus TL1A proteins were both capable of binding and activating the DR3 receptor on human TF-1 cells, resulting in intracellular caspase activation and apoptosis. A219 inhibited human and monkey TL1A-induced caspase activation in TF-1 cells with IC50 values of 0.27 nM and 0.59 nM, respectively.

PBMCs in whole blood collected from cynomolgus monkeys release IFN-γ when stimulated with immune complex in the presence of IL-12 and IL-18. This enhancement of IFN-γ secretion reflects immune complex-driven TL1A production by PBMCs. The ability of A219 to inhibit IFN-γ release under these conditions was assessed in vitro in freshly collected monkey whole blood.

IFN-γ levels were measured in whole blood after stimulation in vitro with immune complex in combination with IL-12 and IL-18, and in the presence of increasing concentrations of A219 (concentration range 0.05 nM to 100 nM). IFN-γ release was inhibited by A219 in a dose-dependent manner in monkey whole blood. The mean IC50 and IC90 values for the inhibition of the IFN-γ response were 1.54 nM (289 ng/ml) and 17.7 nM (3321 ng/mL), respectively.

### Example 14: In Vivo Pharmacology

In a single dose PK/PD study with an 11 day follow on period in monkeys, A219 was administered by IV bolus to 3 animals/group (mixed sexes) at doses of 0 (i.e., 0.56 mg/kg human IgG1 isotype control), 0.0056, 0.056 and 0.56 mg/kg. The A219 doses tested in the study were selected to result in A219 serum concentrations of approximately 1-, 10-, or 100-fold of the IC50 based on results from the in vitro monkey whole blood IFN-γ assay. Blood was collected to assess PK, sTL1A concentrations, and in vitro whole blood IFN-release. The effect of A219 on the inhibition of TL1A-mediated IFN-γ release at 0.0056 mg/kg could not be evaluated due to the insufficient increase in IFN-γ release at the pre-dose baseline. Administration of 0.056 mg/kg or 0.56 mg/kg A219 resulted in nearly full inhibition of IFN-γ release at 1-hour post-dose, relative to the isotype control. At 264 hours (11 days) post-dose, inhibition of IFN-γ release was less than at 1 hour post-dose but persisted in the 0.56 mg/kg A219 group. Inhibition of TL1A-mediated IFN-γ release was dose-dependent at doses≥0.056 mg/kg where the observed exposure at 0.056 mg/kg was ≥6.8-fold above the in vitro whole blood assay IC50 of 1.54 nM (289 ng/mL).

Following administration of 0.0056, 0.056 or 0.56 mg/kg A219, the mean concentration of sTL1A increased in a dose-dependent manner, by 3.6-, 10.4-, and 14.4-fold, respectively, relative to the isotype control antibody at 6 hours post-dose (FIG. 7B). The increase in TL1A concentrations across all A219 dose groups was observed at the earliest timepoint tested (6 hours post-dose) and was sustained until the last timepoint (264 hours post-dose).

### Safety Pharmacology

Cardiovascular, CNS, and respiratory safety pharmacology endpoints were incorporated into a 6-week repeat-dose IV toxicity study in monkeys.

There were no functional effects on the cardiovascular system as assessed by ECG measurements in the pre-dose phase and during Weeks 1 and 6 of the dosing phase, and by microscopic evaluation of the heart and major blood vessels at 300 mg/kg/week. There were no changes related to A219 administration in heart rate, no cardiac rhythm abnormalities or qualitative or quantitative ECG changes, and no microscopic findings noted in the heart that would have impacted cardiac function.

There were no functional effects on the CNS based on daily clinical observations and detailed weekly examinations that included: observing the animal's behavior and movement while approaching the cage, autonomic activity (e.g., lacrimation, piloerection, pupil size), changes in posture and reactivity to handling, as well as presence of clonic or tonic movements, behavioral/psychological abnormalities, circling, and self-mutilation at 300 mg/kg/week. There were no microscopic findings in the brain or nervous system that would have impacted the CNS function.

There were no effects on the respiratory system based on daily clinical observations of animal respiration and detailed weekly examinations that included monitoring for unusual respiratory patterns at 300 mg/kg/week.

In conclusion, there were no functional cardiovascular, CNS or respiratory system findings observed in monkeys during 6 weeks of once weekly IV administration of A219 at 300 mg/kg/week.

### Systemic Pharmacokinetics in Animals

The serum PK and toxicokinetics (TK) of A219 were investigated in the monkey, to support dose selection for the pivotal 6-week toxicity study and to aid in projecting the appropriate starting dose in humans. IV dosing was used in all in vivo studies.

A single IV dose PK/PD study in monkeys was performed to characterize the PK profile and the associated PD effects of A219 at dose levels relevant to projecting the first-in-human starting dose. PD results are summarized previously. The PK of A219 was nonlinear over the 0.0056, 0.056, and 0.56 mg/kg dose range, which is consistent with the expected target-mediated drug disposition (TMDD) for a monoclonal antibody to a membrane-bound target. AUC values increased in a greater than dose-proportional manner, and where it could be estimated, t½ increased with increasing dose (Table 20).

TABLE 20
Mean (SD) PK Parameters after a single
IV dose to cynomolgus monkeys

| Dose (mg/kg) | Cmax (μg/mL) | AUC0-t (hr*μg/mL) | t½ (hr) |
|---|---|---|---|
| 0.0056 | 0.142 (0.0181) | 0.411 (0.297) | 12.1^{a} |
| 0.056 | 1.58 (0.245) | 79.7 (13.5) 1200 | 63.6 (11.9) |
| 0.56 | 12.8 (1.35) | (234) | 113^{b} |

Cmax = maximum observed concentration;
AUC0-t = area under the concentration versus time curve from time 0 to the timepoint with the last measurable concentration;
t½ = terminal half-life
N = 3 unless otherwise noted
^{a}N = 1 (insufficient characterization of the terminal phase of the concentration versus time profile to estimate this parameter in the other two animals)
^{b}N = 2 (insufficient characterization of the terminal phase of the concentration versus time profile to estimate this parameter in the third animal)

TK and immunogenicity were evaluated in cynomolgus monkeys as part of a 2-dose non-GLP PK/tolerability study and a GLP 6-week repeat-dose toxicity study.

In the 2-dose PK study, monkeys (N=1/sex/group) received 2 doses of A219 via IV bolus administration one week apart at dose levels of 30, 100, and 243 mg/kg. Following the first (Day 1) or second (Day 8) dose, exposure based on mean Cmax increased in an approximately dose-proportional manner. Mean AUC0-t values increased in a dose-dependent, but not necessarily dose-proportional manner after the first and second dose.

In the GLP 6-week repeat-dose toxicity study, monkeys (N=3-5/sex/group) received A219 via IV bolus administration at dose levels of 0, 30, 100, or 300 mg/kg once weekly for 7 doses. Exposure to A219 was comparable in male and female monkeys following single and repeat dosing (differences in mean Cmax and AUC values were less than 2-fold). A219 exposure increased in an approximately dose-proportional manner after single and repeat dosing. Accumulation was observed after repeat once weekly dosing at all dose levels (serum exposure after the last dose (Day 42) was approximately 1.5 to 2.3-fold higher than that observed after the first dose; Table 21).

TABLE 21
Mean (SD) TK Parameters after repeat once
weekly IV dosing to cynomolgus monkeys

| | | | Dose | | |
|---|---|---|---|---|---|
| Day | Parameter | Units | 30 mg/kg | 100 mg/kg | 300 mg/kg |
| 1 | Cₘₐₓ | μg/mL | 743 (115) | 2500 (433) | 8760 (1710) |
| | AUC_{0-24 hr} | hr*μg/mL | 14500 | 45400 (4970) | 154000 (27700) |
| | AUC₀₋ | hr*μg/mL | 61000 | 205000 (37600) | 684000 (110000) |
| | Cₘₐₓ | μg/mL | 1490 (182) | 5530 (1270) | 13800 (3320) |
| | AUC_{0-24 hr} | hr*μg/mL | 28700 | 106000 (10500) | 267000 (82900) |
| | AR_{Cmax} | | 2.12 (0.667) | 2.25 (0.517) | 1.59 (0.421) |
| [symbol] | AR_{AUC0-} | | 2.01 (0.364) | 2.34 (0.141) | 1.73 (0.522) |
| | AUC₀₋ₜ^{a} | hr*μg/mL | N/A | N/A | 1600000 (970000) |
| | [symbol] | hr | N/A | N/A | 38.7 (3.76) |

Mean values were calculated based on males and females combined.
Cmax = maximum observed concentration; AUC0-24 hr = area under the concentration versus time curve from time 0 to 24 hr postdose; AUC0-168 hr = area under the concentration versus time curve from time 0 to 168 hr postdose; AUC0-t = area under the concentration versus time curve from time 0 to the timepoint with the last measurable concentration; ARCmax = accumulation ratio based on Cmax; ARAUC0-24 hr = accumulation ratio based on AUC0-24 hr; t½ = terminal half-life N = 3 males and 3 females in the 30 and 100 mg/kg dose groups and 5 males and 5 females in the 300 mg/kg dose group.
^{a}Estimated in recovery animals only (N = 2/sex). [symbol]
indicates data missing or illegible when filed

### Example 15: Toxicology

A219 was assessed in a series of in vitro and vivo toxicity studies outlined in Table 22. The IV route of exposure was selected for the in vivo studies. The weekly dosing regimen used in the definitive 6-week repeat-dose monkey toxicity study was selected based on the half-life of A219 in monkeys and was designed to have a similar or more intensive dosing regimen than the clinical dosing regimen.

TABLE 22
Overview of the toxicology program

| Study | Concentrations or Doses | |
|---|---|---|
| 6-Week IV Toxicity in Monkeys with a 6- | 0, 30, 100, 300 | mg/kg/week |
| Week Recovery | | |
| Tissue Cross-Reactivity in Monkey | 0, 0.625, 1.25, 2.5 | g/mL |
| and Human Tissues | | |
| Cytokine Release Assay Using Human | 0, 0.0002 to 2 | mg/mL |
| Whole Blood | | |
| Cytokine Release Assay Using Human | 0, 0.0002 to 2 | mg/mL |
| PBMCs | | |
| Antibody Dependent Cellular | 0, 0.0031 to 30000 | ng/mL |
| Cytotoxicity Assay | | |
| Complement Dependent Cytotoxicity Assay | 0, 0.0031 to 30000 | ng/mL |

PBMCs = peripheral blood mononuclear cells

The monkey was selected as the pharmacologically relevant nonclinical species because of similar TL1A protein sequence homology and nearly equivalent binding affinity of A219 to monkey TL1A, as compared to human TL1A. A219 was also pharmacologically active with similar IC50 values after binding monkey or human soluble TL1A in an in vitro cell-based assay. In an in vitro assay using monkey whole blood stimulated to express TL1A with subsequently IFN-γ release, the addition of A219 inhibited IFN-γ release in a dose-dependent manner. A similar inhibition of IFN-γ release was observed when blood from monkeys administered A219 was used in the assay. Binding of A219 to mouse or rat TL1A was also assessed and A219 did not bind rat or mouse TL1A.

### 2-Dose PK and Tolerability Study in Monkeys

Tolerability was assessed in a 2-week PK and tolerability study. Monkeys (1/sex/group) were administered A219 IV at 30, 100 or 243 mg/kg/week on Days 1 and 8. A219 was well-tolerated up to the highest dose tested, and the only clinical signs observed in A219-treated animals were loose stools in all dose groups at multiple observation timepoints. Based on the small numbers of animals and no control group animals, the relationship of the loose stools to A219 administration could not be determined. There were no A219-related changes in body weight, clinical chemistry, or hematology parameters.

6-Week Repeat-Dose Study with a 6-Week Recovery in Monkeys

A GLP repeat-dose toxicity study of 6 weeks duration (once-weekly dosing) was conducted with A219 in monkeys. A219 was administered by IV bolus to male and female monkeys (3/sex/group) at doses of 0 (vehicle control), 30, 100, or 300 mg/kg/week (7 doses total). Additional animals (2/sex/group) at 0 and 300 mg/kg/week were assessed after a 6-week recovery period for the reversibility of any A219-related effects.

A219 was well-tolerated after 6 weeks of administration at doses up to 300 mg/kg/week. Based on these studies, NOAEL is 100 mg/kg/week for males and 30 mg/kg/week for females.

### Human Cytokine Release Assays

### PBMC Assay

The potential ability of A219 to trigger cytokine release in primary human PBMCs derived from 10 normal healthy donors was evaluated in soluble and wet-coated formats. A range of A219 concentrations from 0.00002 to 2 mg/mL were evaluated. A human IgG4 antibody and untreated samples were used as negative controls; anti-CD3 (OKT3) antibody was used as positive controls. The levels of IL-2, IL-6, IL-10, TNF, and INF-γ were measured after PBMCs or were cultured with A219 for 24 hours. PBMCs from all donors induced IL-2, IL-6, IL-10, TNF, and IFN-γ release in response to OKT3 treatment (positive control). The IL-2 response of Donor 9 was lower but present. The IgG4 negative control antibody induced no or low IL-2, IL-10, TNF, and IFN-γ cytokine release under any of the tested conditions. The IgG4 negative control antibody induced IL-6 production in several donors, although not as robustly as the positive control treatment. Cytokine release was either not observed or only observed at very low levels in untreated samples from all donors.

A219 did not induce IL-2 and IFN-release under any of the tested conditions. A219 induced low levels of IL-10 and TNF release in some donors, but not above levels induced by the IgG4 negative control antibody and/or in untreated samples. A219 induced IL-6 release in several donors in the same range of induction as observed with the IgG4 negative control and/or in untreated samples in both stimulation formats, but the responses were not concentration dependent. Based on historical testing facility data for IL-6 induction, a variable range of magnitude of responses has been observed for isotype and other negative control antibodies, as well as other test articles in subsets of donors that are often not concentration dependent. A219, IgG4 treatment-related and untreated PBMC responses were lower than the anti-CD3 positive control treatment-related responses. Therefore, the induction of IL-6 in this assay was likely not A219-specific but related to variation that has also been observed historically in the assay for this cytokine.

In conclusion, A219 did not induce IL-2, IL-6, IL-10, TNF, IFN-specific release from PBMCs from 10 different donors in wet-coated plate or soluble formats above that observed for the IgG4 negative control antibody and/or untreated samples.

### Whole Blood Assay

The potential ability of A219 to trigger cytokine release in human whole blood derived from 10 normal healthy donors was evaluated in soluble and wet-coated formats. A range of A219 concentrations from 0.00002 to 2 mg/mL were evaluated. A human IgG4 antibody and untreated samples were used as negative controls; Staphylococcal enterotoxin B (SEB) were used as a positive control. The levels of IL-2, IL-6, IL-10, TNF, and INF were measured after whole blood was cultured with A219 for 24 hours.

Whole blood from all donors induced IL-2, IL-6, IL-10, TNF, and IFN-γ release in response to SEB treatment (soluble stimulation format). The IFN-response of Donors 1, 3, and 8 was lower but present. In whole blood from most donors, the human IgG4 negative antibody control induced no or low cytokine production under all tested conditions. Cytokine release was not observed in untreated whole blood samples of most donors. Whole blood from one donor (Donor 7) produced IL-6, IL-10, and TNF-α in response to several concentrations of soluble IgG4 negative antibody control. However, the cytokine levels were generally at or below the levels observed for the same donor in untreated samples.

A219 did not induce any cytokine release under any of the tested conditions in nine donors. Stimulation with 0.02 mg/mL of soluble A219 induced release of low levels of IL-6, IL-10 and TNF in whole blood from Donor 7. A dose-response relationship between A219 concentration and cytokine levels was not observed for this donor, and the cytokine levels were below those observed with the IgG4 negative control and/or in untreated samples from this donor. Therefore, the induction of IL-6, IL-10 and TNF in Donor 7 samples were likely not A219-specific.

In conclusion, A219 did not induce IL-2, IL-6, IL-10, TNF, IFN-γ specific release in whole blood from 10 different donors in wet-coated plate or soluble formats above that observed for the IgG4 negative control antibody and/or untreated samples.

### Fc Effector Function Assays

The potential for A219 to elicit CDC or ADCC was evaluated in vitro. A219 was not expected to elicit CDC or ADCC because the antibody was designed to abolish effector functions.

The ability of A219 to elicit CDC or ADCC on target-expressing recombinant human HEK293 TL1A cells and on the HEK293 parental cell line (negative control cell line) was evaluated. CDC was assessed by culturing the cells after treatment with a range of concentrations (0.0031 to 30,000 ng/ml) of A219 in the presence of human complement and analyzing the viability of target cells by flow cytometry. ADCC was assessed by culturing labeled target cells, after treatment with a range of concentrations (0.0031 to 30,000 ng/mL) of A219, with human PBMCs (3 donors). A human IgG4 antibody was used as a negative control in both assays.

Rituxan (anti-CD20 antibody) was used as a positive control in the CDC assay with CD20-expressing Raji cells while Darzalex was used in the ADCC assay with Daudi target cells.

A219 treatment did not cause an increase in CDC-mediated cell killing of HEK293 TL1A cells or in HEK293 cells as compared to the negative control antibody. Rituxan treatment resulted in an expected increase in complement-mediated lysis of CD20-expressing Raji cells.

A219 treatment did not cause an increase in ADCC-mediated cell killing of HEK293 TL1A cells or in HEK293 cells as compared to the negative control antibody. Darzalex treatment resulted in an expected increase in ADCC cytotoxicity of Daudi target cells.

In conclusion, and as expected, A219 did not elicit CDC or ADCC of TL1A-expressing cells in the presence of human complement or PBMCs, respectively.

### Relationship of Findings to Pharmacokinetics

A219 exposure in monkeys in the 6-week repeat-dose toxicity study, as defined by Cmax and AUC, increased with increasing dose over the dose range tested, and exposure increased in an approximately dose-proportional manner. There were no apparent sex-related differences in exposure. There was no clear correlation of ADA with changes in A219 exposure. However, it is likely that ADA led to the more rapid decrease in A219 concentrations at later timepoints that was observed in some of the animals. Accumulation of A219 was observed in monkeys after repeated once weekly administration.

The threshold of serum exposures associated with A219-related findings from the 6-week repeat-dose monkey toxicity study is shown in Table 23. Safety margins at each dose level are presented based on a comparison of A219 AUC values from the 6-week repeat-dose monkey toxicity study in comparison to projected human AUC values at the proposed clinical starting dose of 5 mg.

TABLE 23
Exposure of anti-TLIA associated with findings in
a 6-week repeat-dose toxicity study in monkeys.

| | Dose | AUC_{0-168 h} | Cmax | Exposure^{a} |
|---|---|---|---|---|
| Study Findings | (mg/kg/week) | (ug*hr/mL) | (ug/mL) | Margin |
| Mortality (1 female) | 30 | 61000 | 743 | 425 |
| Perivascular infiltrates | | | | |
| As above and: | 100 | 205000 | 2500 | 1430 |
| Vascular inflammation | | | | |
| As above and: | 300 | 684000 | 8760 | 4770 |
| RBC and albumin, globulin, increased | (NOAEL) | | | |
| spleen weight (females only), glomerulopathy | | | | |

AUC = area under concentration-time curve;
RBC = red blood cells;
Cₘₐₓ = Maximum observed concentration
^{a}Exposure margins (i.e. safety margins) were calculated by dividing AUC₀₋₁₆₈ values in the repeat-dose monkey study by the projected human AUC0-168 value of 143.5 ug*hr/mL at the projected 5 mg human starting dose.

### Summary of A219 Preclinical Studies

A219 has sub-nanomolar binding affinity to soluble TL1A and nanomolar affinity to membrane-associated TL1A. In in vitro studies, A219 blocked TL1A's ability to bind and activate its receptor, DR3. In whole blood, A219 inhibited the TL1A-dependent IFN-γ response following the ex vivo exposure to immune-complex and a combination of IL-12 and IL-18. Additionally, A219 was observed to be highly selective for TL1A with no detectable binding to related TNF super family members FAS, LIGHT, or TRAIL.

The potential toxicity of A219 was assessed in a series of nonclinical in vitro assays and in vivo studies in cynomolgus monkeys. The monkey was selected as a pharmacologically relevant nonclinical species because of similar TL1A protein sequence homology and nearly equivalent binding affinity of A219 to monkey TL1A, as compared to human. A219 is similarly active in monkey and human in vitro cell-based assays.

A219 has been engineered to remove the potential for the mAb to induce an immune response. In in vitro assays, A219 treatment did not lead to antibody- or cell-mediated cytotoxicity or cytokine release from peripheral blood cells thus indicating that it was not provoking an undesired immune response.

In a tolerability and pharmacokinetic (PK) study, cynomolgus monkeys (1/sex/group) were administered A219 intravenous (IV) at 30, 100 and 243 mg/kg/week on Days 1 and 8 and subsequently followed for approximately 11 weeks to assess systemic exposure of A219. There were no A219-related clinical observations or changes in body weight, clinical chemistry, or hematology parameters. PK measurements suggested that A219 has a long half-life of 5 to 11 days, which is consistent with human IgG1 in monkeys.

A GLP study was performed to evaluate the potential toxicity, including immunotoxicity, of A219 and associated systemic exposure after six weeks of once-weekly dosing (7 total doses) in cynomolgus monkeys. A219 was administered IV (bolus) to male and female monkeys (3/sex/group) at doses of 0 (vehicle control), 30, 100, or 300 mg/kg/week. Recovery animals (2/sex/group) were administered 0 or 300 mg/kg/week of A219. The clinically relevant no observed adverse effect level (NOAEL) in this study was determined to be 300 mg/kg/week (the highest dose tested). There were findings observed that were secondary to generation of anti-drug antibodies (ADA) in response to administration of a humanized monoclonal antibody including a single death in the 30 mg/kg group and minimal vascular inflammation, which was the only finding that was considered adverse. All findings were fully reversible after a 6-week recovery period except for perivascular infiltrates, which persisted minimally in only a few tissues at 300 mg/kg/week, and minimal glomerulopathy noted in one recovery female at 300 mg/kg/week. ADA-related findings observed in nonclinical animal toxicity studies with human monoclonal antibodies generally are not considered relevant to humans.

A six-month repeat-dose monkey toxicity study is performed to evaluate the potential for chronic dosing in UC and CD.

### Example 16: Phase 1 Clinical Trial

A Phase 1a clinical trial in normal healthy volunteers has begun.

The Phase 1a clinical trial is a single-center, double-blind, placebo-controlled safety, tolerability and PK study in normal healthy volunteers receiving IV administration of A219. The single ascending dose (SAD) phase of the trial consist of 8 subjects (6 active and 2 placebo) per cohort with up to 6 dose levels. The multiple ascending dose (MAD) phase of the trial commences after an equal or higher SAD dose has been studied and acceptable safety and tolerability has been observed. The MAD phase consists of 8 subjects (6 active and 2 placebo) per cohort with up to 5 dose levels. The trial evaluates the safety, tolerability and pharmacokinetics of single and multiple doses of A219 via IV administration as well as the PK of A219 after single and multiple doses in healthy, ambulatory, non-smoking, male or female volunteers aged 18 to 60 years. In addition, the trial determines the effects of A219 on pharmacodynamic (PD) markers as well as the exposure-response relationship of A219 on PD markers. A synopsis of this study is shown in Table 14.

A Phase 1b/2a randomized placebo-controlled clinical trial in patients with moderate-to-severe UC and an open label Phase 1b clinical trial in patients with moderate-to-severe CD is conducted.

TABLE 14
Synopsis of Phase 1, Single-Center, Double-Blind, Placebo-Controlled, Safety and
Pharmacokinetics Study of anti-TLIA antibody in Healthy Volunteers

| | |
|---|---|
| OBJECTIVE: | To assess: |
| | The safety and tolerability of single and multiple doses of anti- |
| | TLIA antibody following administration. |
| | The pharmacokinetics (PK) of anti-TLIA antibody after single |
| | and multiple doses. |
| | The effects of anti-TLIA antibody on tissue and serum |
| | pharmacodynamic (PD) markers. |
| | The exposure-response relationship of anti-TLIA antibody on |
| | PD markers. |
| STUDY DESIGN: | Single center, double-blind, randomized, placebo-controlled, single dose |
| | followed by multiple dose study of anti-TLIA antibody. |
| SAMPLE SIZE: | Single Dose Phase: |
| | Eight (8) subjects (6 active, 2 placebo) per dose level; up to 6 |
| | dose levels |
| | Multiple Dose Phase: |
| | Eight (8) subjects (6 active, 2 placebo) per dose level; up to 5 |
| | dose levels |
| | Subjects who discontinue the study prematurely may be replaced. |
| SUBJECT TYPE: | Healthy, ambulatory, non-smoking, male or female volunteers aged 18 |
| | to 60 years. Female volunteers must be women of non-childbearing |
| | potential. |
| DOSAGE AND DOSE | Single Ascending Dose (SAD) Phase: |
| PROGRESSION: | Placebo (matching volume of 0.9% normal saline [NS]) |
| | anti-TLIA antibody: |
| | Dose Progression: second and higher dosing cohorts to be selected based |
| | on AEs and available PK and PD data |
| | Multiple Ascending Dose (MAD) Phase |
| | Placebo (matching volume of 0.9% NS) |
| | anti-TLIA antibody on Day 1/Weeks 0, Day 15/Week 2, and |
| | Day 29/Week 4 |
| | Dosing Progression: second and higher dosing cohorts to be |
| | selected based on AEs and available PK and PD data. |
| STUDY | Adverse events, physical examinations, chest x-ray, vital signs, |
| PARAMETERS: | ECGs, clinical laboratory values, and anti-drug antibody levels in serum |
| | samples. |
| | Pharmacokinetics: Concentrations of anti-TLIA antibody in |
| | serum samples will be determined by validated LCMS methods. |
| | Pharmacodynamics: Change from Baseline in serum and tissue |
| | (in the MAD cohorts where sigmoidoscopy will be performed) |
| | PD markers |
| INCLUSION | Subjects are required to meet the following criteria in order to be |
| CRITERIA: | included in the study: |
| | 1. Male or female (of non-childbearing potential only) between 18 |
| | and 60 years of age. |
| | 2. Females must be of non-childbearing potential and must have |
| | undergone one of the following sterilization procedures, and have |
| | official documentation, at least 6 months prior to the first dose: |
| | a. hysteroscopic sterilization; |
| | b. bilateral tubal ligation or bilateral salpingectomy; |
| | c. hysterectomy; |
| | d. bilateral oophorectomy, or; |
| | e. be postmenopausal with amenorrhea for at least 1 year prior to |
| | the first dose and have FSH serum levels consistent with |
| | postmenopausal status as per investigator judgment. |
| | Note: A female of non-childbearing potential who has undergone one of |
| | the sterilization procedures mentioned above, but could not provide |
| | official documentation, must be sexually inactive and remain inactive |
| | throughout the study, or must agree to use a physical (e.g., condom, |
| | diaphragm) and a chemical (e.g., spermicide) barrier method from the |
| | time of screening and throughout the study. |
| | 3. Male subjects must use reliable forms of contraception from |
| | screening to 30 days after the end of dosing. |
| | Note: A non-vasectomized, male subject must agree to use a condom |
| | with spermicide or abstain from sexual intercourse during the study until |
| | 30 days beyond the last dose of study drug. (No restrictions are required |
| | for a vasectomized male provided his vasectomy has been performed 4 |
| | months or more prior to the first dose. A male who has been |
| | vasectomized less than 4 months prior to the first dose, or could not |
| | provide official documentation, must follow the same restrictions as a |
| | non-vasectomized male). |
| | 4. Continuous non-smoker who has not used tobacco or nicotine- |
| | containing products for at least 6 months prior to the first dose of study |
| | drug. |
| | 5 Good general health as determined by medical history, and by |
| | results of physical examination, chest x-ray, vital signs, ECG, and |
| | clinical laboratory tests obtained within 28 days (4 weeks) prior to study |
| | drug administration. |
| | 6. Subjects must have documentation of positive serology for |
| | varicella zoster virus (VZV) immunoglobulin G (IgG) antibody status. |
| | 7. Able to provide written informed consent and understand and |
| | comply with the requirements of the study. |
| EXCLUSION | Subjects with the following characteristics will be excluded from the |
| CRITERIA: | study: |
| | 1. Subject participation in more than one cohort. |
| | 2. History or presence of any clinically significant organ system |
| | disease that could interfere with the objectives of the study or the safety |
| | of the subjects. |
| | 3. Blood pressure and heart rate are outside the ranges 90-140 |
| | mmHg systolic, 40-90 mmHg diastolic, heart rate 60-100 beats/min. |
| | 4. 12-lead ECG with any abnormality judged by the Investigator to |
| | be clinically significant, QRS ≥ 110 milliseconds (msec), or QT/QTcF |
| | interval of > 450 msec for men or >470 msec for women. |
| | 5. Presence or history of any abnormality or illness, which in the |
| | opinion of the Investigator may affect absorption, distribution, |
| | metabolism or elimination of the study drug. |
| | 6. Any screening laboratory evaluation outside the laboratory |
| | reference range that is judged by the Investigator to be clinically |
| | significant. |
| | 7. History of or current active tuberculosis (TB) infection; history |
| | of latent TB that has not been fully treated or current latent TB infection. |
| | 8. History of more than one episode of herpes zoster infection or |
| | history of disseminated herpes zoster infection. |
| | 9. Positive serum test for HIV, hepatitis C or hepatitis B virus |
| | infection. |
| | 10. History of significant allergy to any medication. |
| | 11. History of alcohol or drug abuse within the past 24 months. |
| | 12. Administration of any prescription drug within 21 days of study |
| | drug administration; or over-the-counter drug (acetaminophen and |
| | ibuprofen ≤ 1 g/day permitted) or herbal, nutritional or vitamin |
| | supplement within 7 days of study drug administration. |
| | 13. Evidence of drug abuse on urine testing, or a positive test for |
| | alcohol. |
| | 14. Administration or use of any investigational drug or device |
| | within 30 days of study drug administration. |
| | 15. Blood or plasma donation within 60 days prior to dosing. |

Based on the clinical data from SAD cohorts 5, 25, 100, 300, and 600 mg and MAD cohort 50 mg, A219 PK exhibits target-mediated drug disposition (TMDD) at lower doses and linear PK at higher dose levels after the saturation of the target-mediated route of clearance.

Following a single IV dose of A219, median time to maximum concentration (Tₘₐₓ) values ranged from 1.02 to 1.50 hours postdose. Exposure based on mean Cₘₐₓ and AUC₀₋ₜ increased in a greater than dose-proportional manner between the 5, 25, and 100 mg dose levels and in an approximately dose-proportional manner between the 100, 300, and 600 mg dose levels. After repeat once every other week dosing of 50 mg A219, exposure was increased relative to Day 1. Mean Cₘₐₓ and AUC_{0-336 hr} was approximately 1.3 times higher on Days 15 and 29 compared to Day 1.

### Example 17: Human Dosing Range and Safety Margins

A219 is a humanized monoclonal antibody that binds human TL1A. Itis expected that the ultimate goal of A219 treatment in humans will be to saturate the TL1A target in disease patients to obtain optimal efficacy. A minimum anticipated biological effect level (MABEL) approach is not considered appropriate because A219 is an antagonist rather than an agonist antibody, and the safety of antagonizing the TL1A pathway has already been established in the clinic. The maximum recommended starting dose for A219 was chosen based on the predicted pharmacologically active dose (PAD). A219 does not cross-react with murine TL1A but binds with approximately equivalent potency to cynomolgus monkey TL1A. Therefore, cynomolgus monkey was considered the relevant species for scaling nonclinical A219 pharmacokinetics (PK) to human. Additionally, PK data in the monkey suggest that A219 exhibits target mediated drug disposition, which leads to nonlinear PK in the dose range where the target is not saturated and linear PK in the dose range where the target is saturated.

The nonclinical PK and TK of A219 were characterized in the monkey and support the proposed once every other week dosing regimen in humans. Estimated safety margins for the starting dose of 5 mg and the highest proposed dose for study, 1000 mg, relative to the GLP safety data from monkeys are shown in Table 24. This table compares the safety margins derived from various approaches.

TABLE 24
Predicted safety margins for the starting dose and maximum dose

| | Predicted Safety Margin Based on a NOAEL | |
|---|---|---|
| | in Monkeys of 300 mg/kg^{a} | |
| Basis for Calculation | 5 mg | 1000 mg |
| Dose | 4200 | 21.0 |
| Cₘₐₓ | 4920 | 22.9 |
| AUC_{0-168 hr} | 4770 | 13.5 |

Cₘₐₓ = maximum observed concentration;
AUC_{0-168 hr} = area under the concentration versus time curve from time 0 to 168 hr postdose
^{a}Predicted human Cₘₐₓ values for the 5 and 1000 mg doses are approximately 1.78 and 382 μg/mL, respectively; predicted human AUC_{0-168 hr} values for the 5 and 1000 mg doses are approximately 143.5 and 50700 hr*μg/mL, respectively. Human exposure parameter values are based on an assumed body weight of 70 kg.

### Example 18: Treatment of IBD with Anti-TL1A

Subjects having inflammatory bowel disease are treated with the anti-TL1A antibody A219 using one of the two induction methods of this example:

Induction method 1: subcutaneous administration of 800 mg of anti-TL1A on day 1, then weekly at 175-200 mg anti-TL1A for 12 total weeks.

Induction method 2: intravenous administration of 500 mg of anti-TL1A every other week for 12 weeks.

After the induction period, if the subject is responsive to treatment, the subject is further treated in a maintenance phase. The maintenance phase comprises administering 175-200 mg of anti-TL1A every 2 or 4 weeks.

### Example 19: Anti-TL1A Antibody Binding to Both TL1A Monomer and TL1A Trimer

To demonstrate that the exemplary anti-TL1A antibody A219 binds to both TL1A monomer and TL1A trimer, a peak shifting assay with size exclusion chromatography was performed. Briefly, recombinantly produced human TL1A (rhTL1A) was labeled with Alexa fluor 488 (AF488) and spiked into normal human serum (NHS). The labeled rhTL1A in serum was then injected into a size exclusion column and eluted by monitoring the AF488 fluorescent signal.

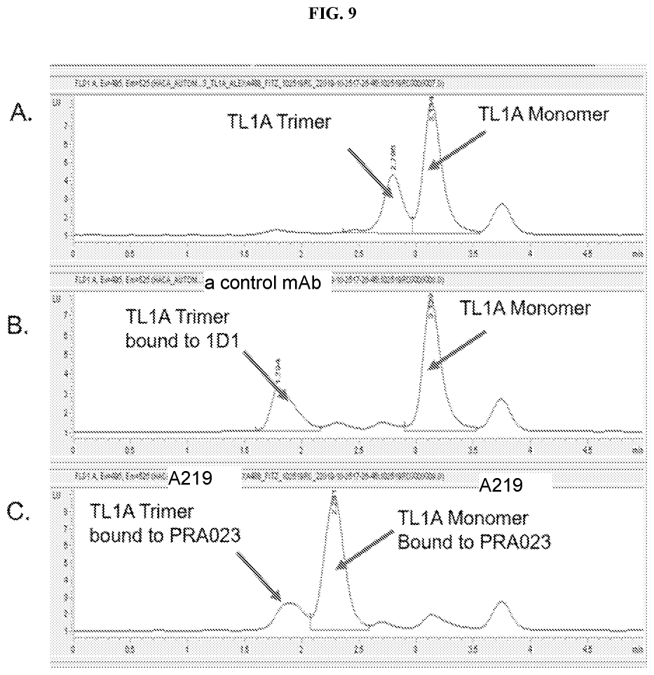

RhTL1A was observed in at least two peaks for two distinct quaternary structures, one for non-covalent trimers and one for monomers (FIG. 9A). The results show that a control reference antibody only bound to the trimeric TL1A (FIG. 9B), as only the trimer TL1A peak shifted in the presence of the control reference antibody (control reference antibody sequence, light chain SEQ ID NO: 382 and heavy chain SEQ ID NO: 383). In contrast, A219 bound both TL1A trimers and monomers (FIG. 9C), as both the monomer and trimer TL1A peaks shifted in the presence of A219. The results demonstrate that the exemplary anti-TL1A antibody A219 binds to both TL1A monomer and TL1A trimer.

### Example 20: PK/PD Models for Determining Effective Dose

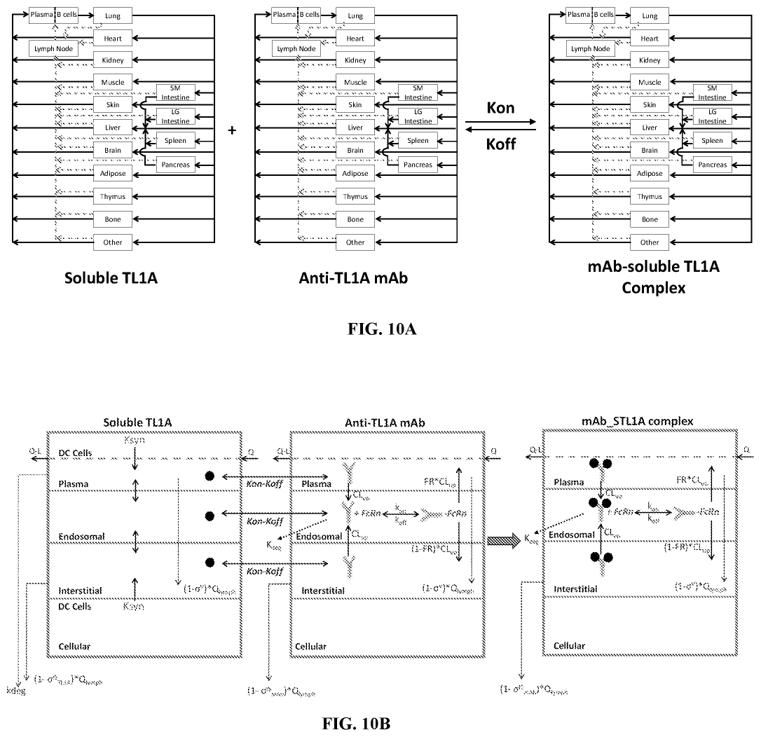

To demonstrate using PK/PD models for determining the effective dose, an integrated whole-body physiologically based pharmacokinetic (PBPK) was established, as shown in FIG. 10A. The integrated whole body PBPK included a tissue-level diagram, as shown in FIG. 10B, to characterize the PK of mAb, ligand, and complex between mAb and ligand. The integrated whole-body PBPK model included the following drug-specific parameters and/or input: (i) soluble TL1A (sTL1A) is synthesized by immune cells (e.g., dendritic cells) all over the body; (ii) monomeric sTL1A has half-life of 20 minutes, and trimeric sTL1A has half-life of 1 hour; (iii) affinity parameters (including the on rate and off rate) between antibody and sTL1A were fixed to the values measured via SPR (e.g., as determined in Example 12); (iv) synthesis rate of sTL1A was adjusted to match the observed baseline and PK data; (v) in the diseased individual, the production rate of sTL1A was increased by 50-fold in the interstitial space of the intestine. The parameters and input can be varied as described herein, including in Section 4.

The whole-body PBPK model recapitulated the PK observations for A219 and for TL1A in normal healthy volunteers (NHV). As shown in FIG. 11A, the A219 concentration predicted by the whole-body PBPK model matched the observed PK for A219 in NHV. Furthermore, as shown in FIG. 11B, the TL1A concentration predicted by the whole-body PBPK model matched the observed TL1A concentration in NHV during the observed time course (assuming constant TL1A production rate).

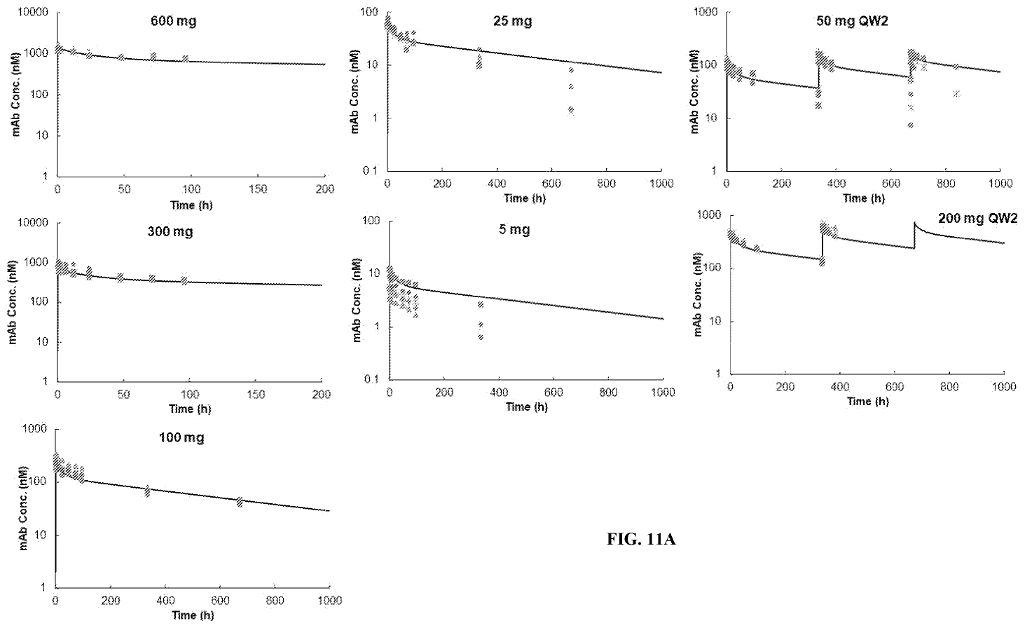

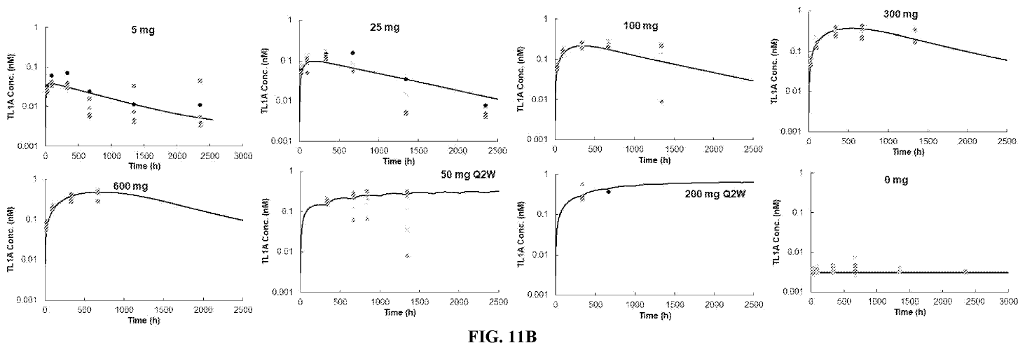

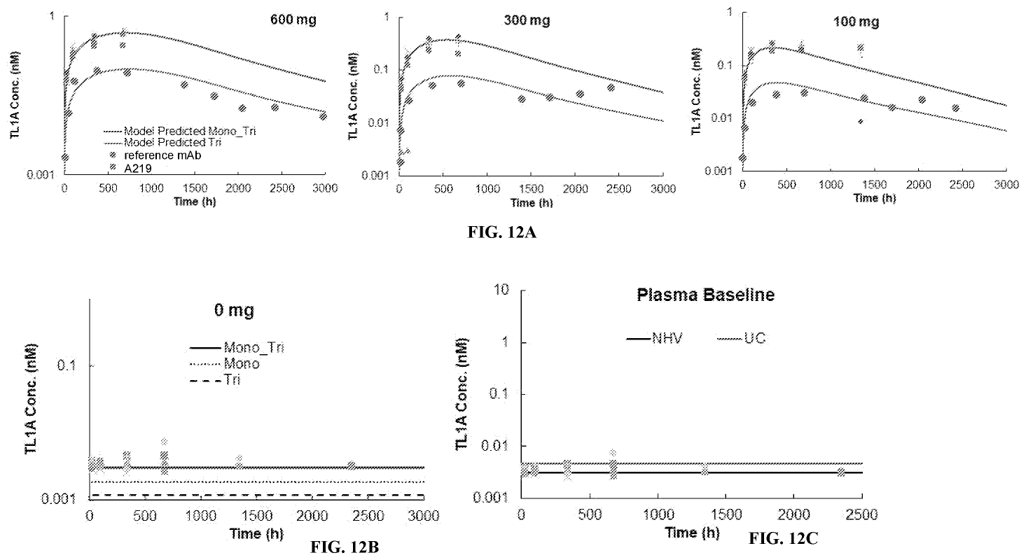

The observed serum concentration of TL1A was almost 10 fold higher when A219, an anti-TL1A antibody binding to both monomeric and trimeric TL1A, was injected, comparing to that when a control reference antibody that binds to only trimeric TL1A antibody was injected in to a subject (FIG. 12A) (control reference antibody sequence, light chain SEQ ID NO: 382 and heavy chain SEQ ID NO: 383). Such higher serum TL1A concentration was recapitulated in the whole-body PBPK, as shown in the curves in FIG. 12A. Furthermore, the model predicted about 40% monomer TL1A and 60% trimeric TL1A, consistent with observations (FIG. 12B). FIG. 12A thus established that treating patients with an anti-TL1A antibody that binds to both monomer and trimer TL1A sequestered 10 fold higher TL1A into the serum, therefore reduced the TL1A concentration in diseased tissues more than an anti-TL1A antibody binding to trimer TL1A alone. Such sequestration of more total TL1A (both monomer and trimer) in serum provides an unexpected advantage to a patient who needs to reduce TL1A concentration in diseased tissues, both in magnitude and rate of such TL1A reduction.

The baseline concentration of TL1A in serum from NHV averaged to about 220 ng/ml (162 to 414 ng/mL, 54 subjects, across ˜110 samples). The baseline concentration of TL1A in serum from CD subjects averaged to about 273 (158 to 479 ng/mL, 17 CD subjects). Thus the difference of serum TL1A concentration between a NHV and a CD patient is only modest, confirming the importance of targeting and reducing the concentration of soluble TL1A in the diseased tissue. Assuming all TL1A production come from colon, the model determined that a 50 fold over-production in colon would reproduce a serum concentration of 290 ng/mL TL1A, approximating the observations in UC patient (FIG. 12C). Such big difference in TL1A in diseased tissue and the modest corresponding difference in serum between NHV and UC patients again highlight the importance of targeting and reducing the concentration of soluble TL1A in the diseased tissue.

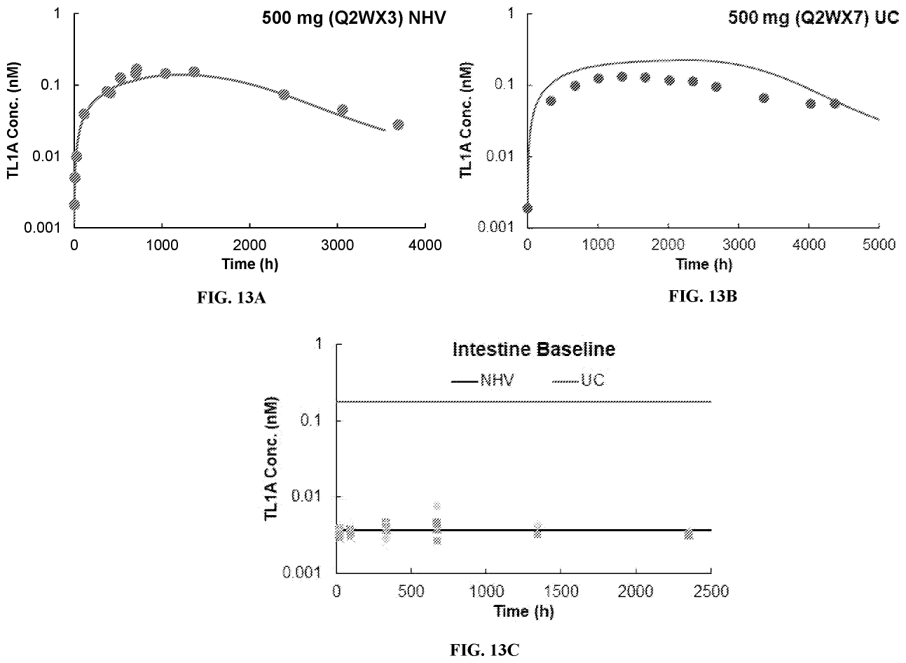

To further validate and establish the applicability of the whole-body PBPK model, the predicted curves of TL1A concentration in serum of NHVs and UC patients were compared with the observations from clinical trials. As shown in FIGS. 13A-13B, the whole-body PBPK model consistently predicted the observations of total TL1A serum concentration in NHVs and UC patients from reported phase I and phase II clinical trials (Banfield C. et al., Br J Clin Pharmacol. 2020 April; 86 (4): 812-824; and Danese S et al., Clin Gastroenterol Hepatol. 2021 Jun. 11; S1542-3565 (21) 00614-5). As shown in FIG. 13C, the whole-body PBPK model also predicted tissue interstitial space TL1A levels in the NHV (normal tissue production) and UC patients (50 fold increase in local tissue production) in the absence of any administration of anti-TL1A antibodies. Thus the fitness of the whole-body PBPK model has been validated by the clinical observations.

Having established the whole-body PBPK model, the whole-body PBPK model was used to simulate the concentration of TL1A in diseased tissues and in serum with various scenarios of TL1A over-production in the diseased tissues, in the presence or absence of various doses of anti-TL1A A219. As shown in FIGS. 14A-14B, the whole-body PBPK model simulated TL1A concentration in intestine for various level of TL1A over-production in intestine (FIG. 14A) and the corresponding serum (plasma) concentration of TL1A under these levels of intestinal TL1A over-production, each in the absence of any administrations of any anti-TL1A antibodies.

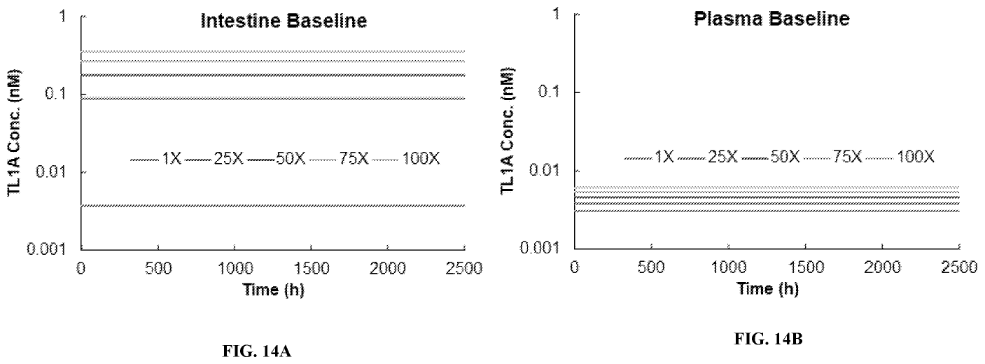

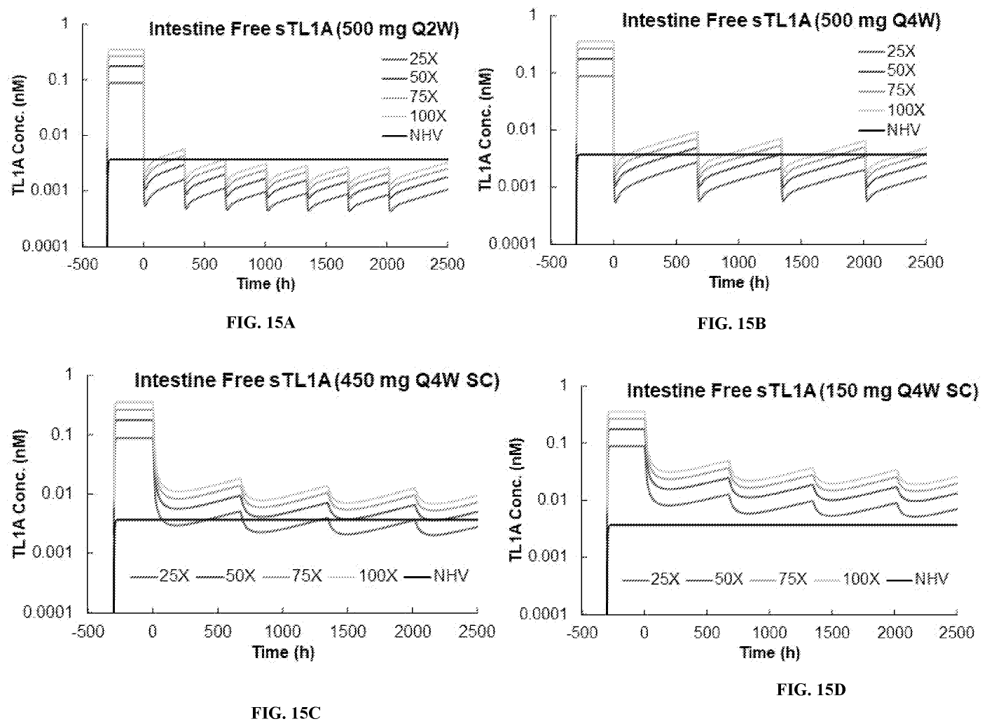

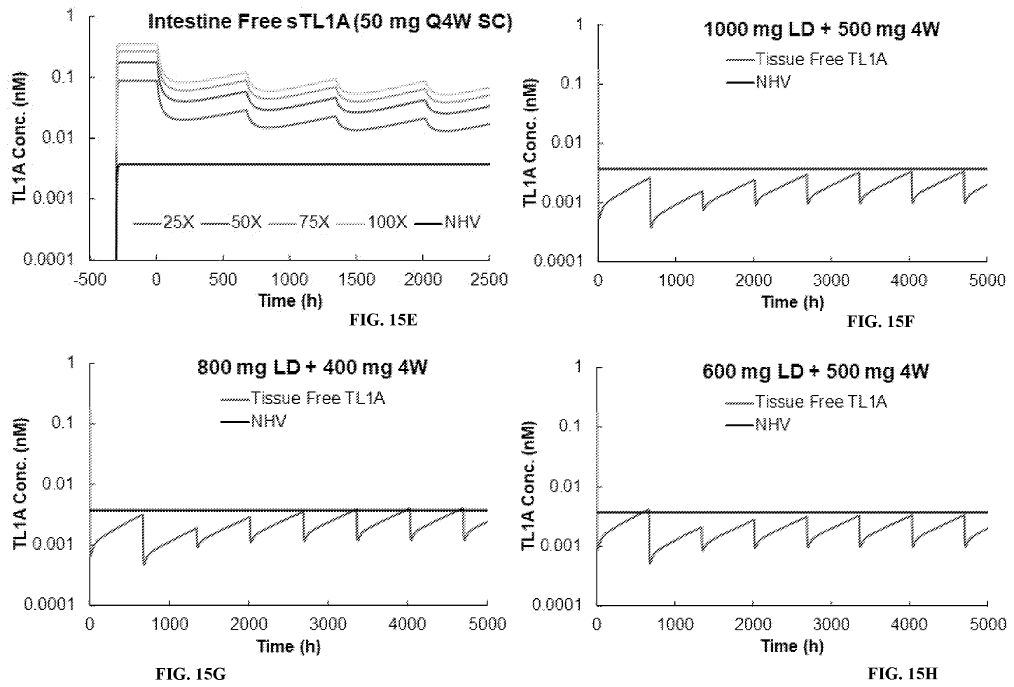

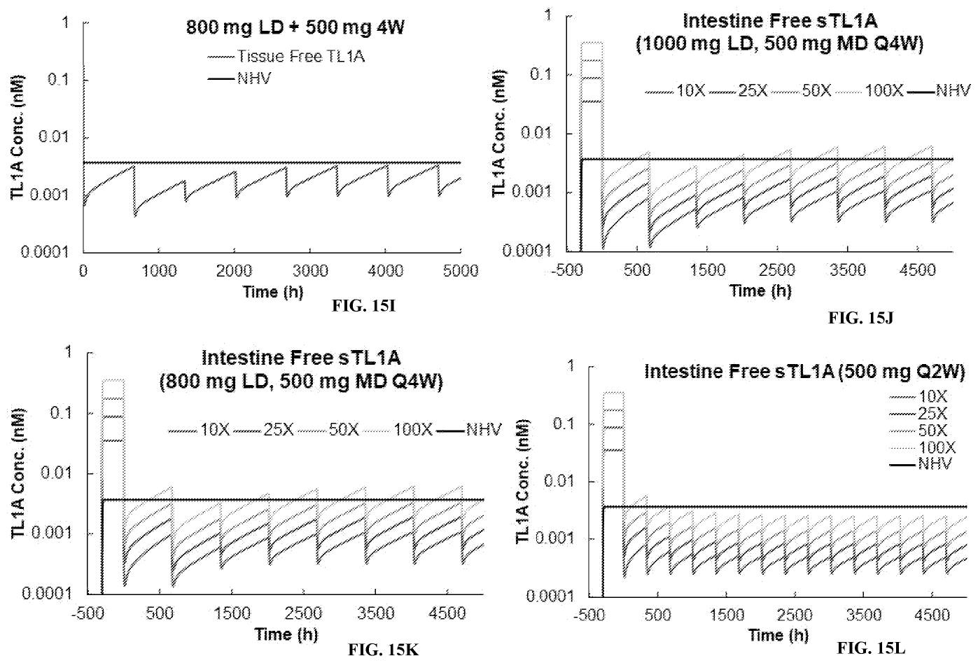

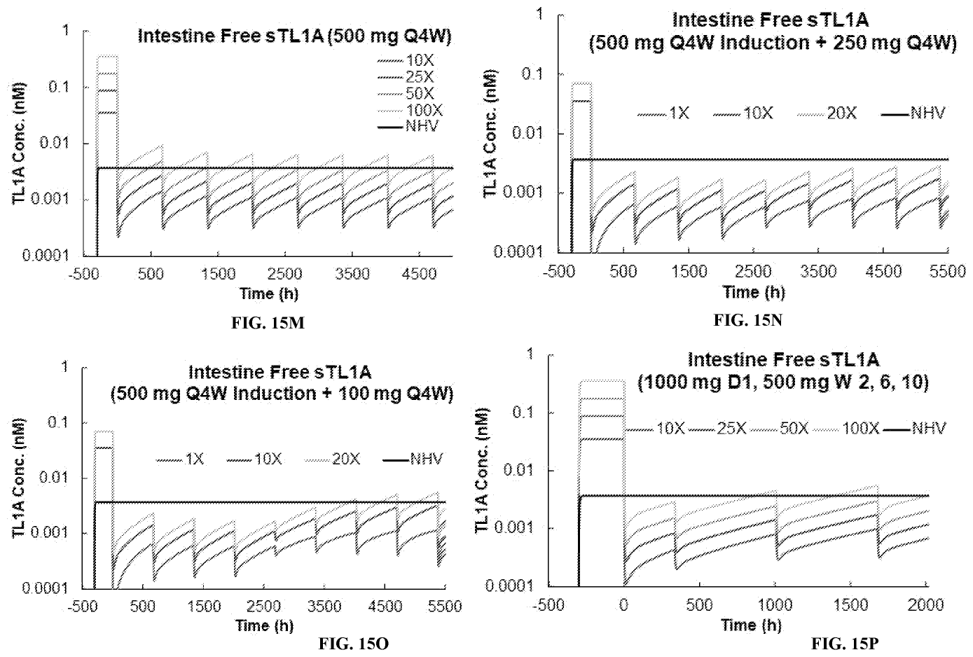

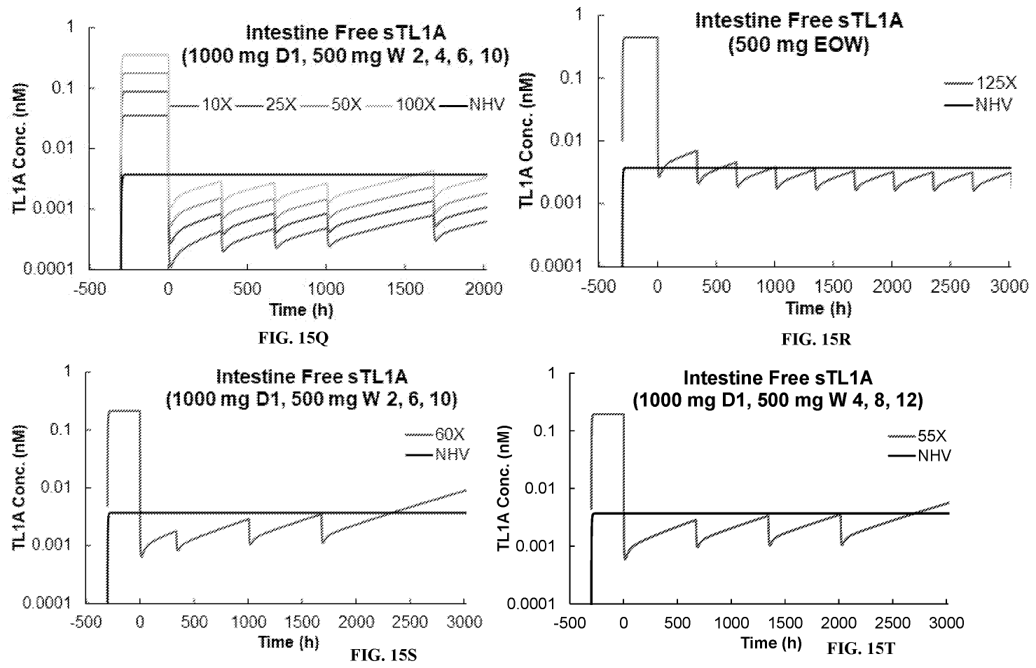

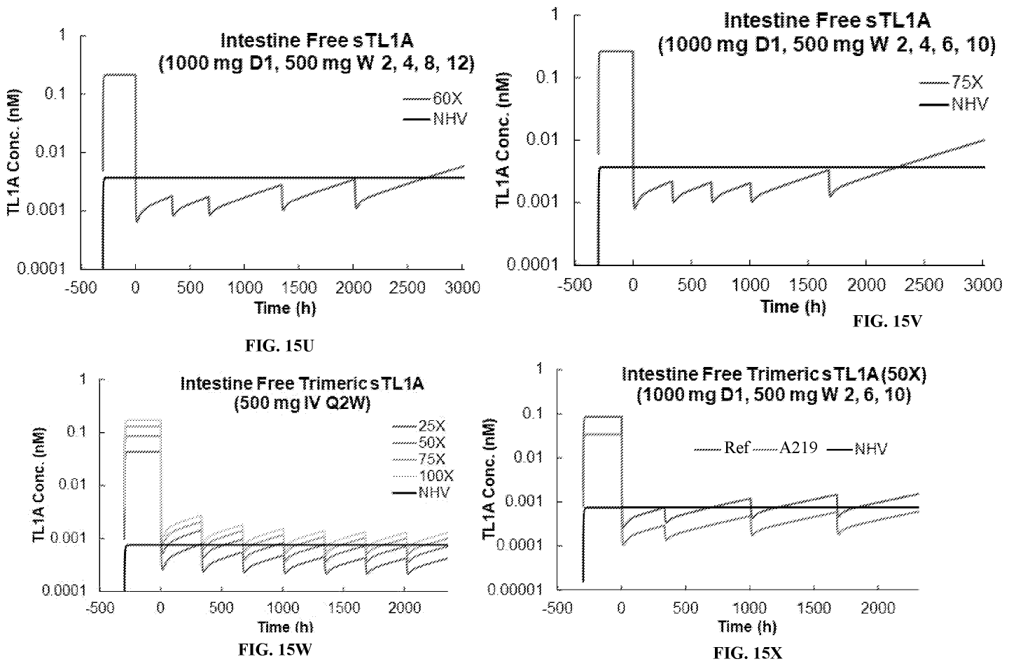

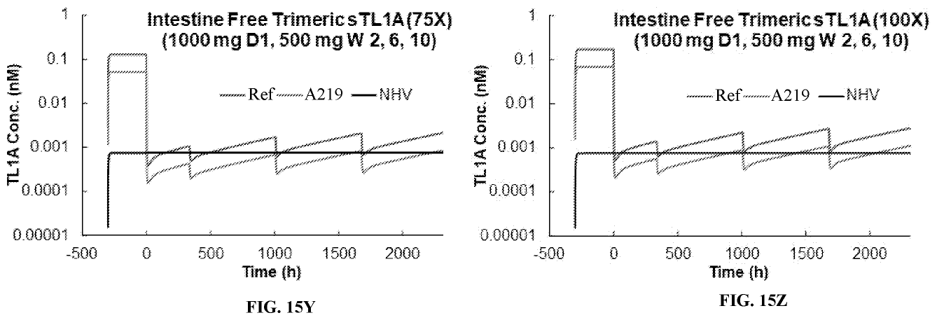

When anti-TL1A antibody A219 was injected at various dose, the whole-body PBPK model simulated the changes of TL1A concentration in the diseased tissues over time (FIGS. 15A-15U). Such simulation can be plotted against the TL1A concentration in the corresponding tissue or a reference tissue of a NHV to determine whether the dose is sufficient to reduce the TL1A concentration in the diseased tissue below the TL1A concentration in the corresponding tissue or a reference tissue of a NHV (FIGS. 15A-15U). FIGS. 15A-15U also depicts such simulation with various parameters of TL1A over-production in the diseased intestinal tissues (10×, 25×, 50×, or 100× fold over-production or fold increase). As shown in FIGS. 15A-15U, the higher the fold over-production, the higher the dose or more administrations of the anti-TL1A antibody A219 are needed to reduce and keep the TL1A concentration in diseased intestinal tissues below that of the NHV for the duration indicated in the figures. More specifically, as shown in FIG. 15R, administration of 500 mg of the anti-TL1A antibody A219 every other week can cover up to about 125 fold over-production (fold increase) of TL1A in the intestine of a patient. As shown in FIG. 15S, administration of a dose at 1000 mg D1, 500 mg W2, W6, W10, (i.e. 100 mg at day 1, 500 mg at week 2, 500 mg at week 6, and 500 mg at week 10) of the anti-TL1A antibody A219 can cover up to about 60 fold over-production (fold increase) of TL1A in the intestine of a patient. As shown in FIG. 15T, administration of a dose at 1000 mg D1, 500 mg W4, W8, W12, (i.e. 1000 mg at day 1, 500 mg at week 4, 500 mg at week 8, and 500 mg at week 12) of the anti-TL1A antibody A219 can cover up to about 55 fold over-production (fold increase) of TL1A in the intestine of a patient. As shown in FIG. 15U, administration of a dose at 1000 mg D1, 500 mg W2, W4, W8, W12, (i.e. 1000 mg at day 1, 500 mg at week 2, 500 mg at week 4, 500 mg at week 8, and 500 mg at week 12) of the anti-TL1A antibody A219 can cover up to about 60 fold over-production (fold increase) of TL1A in the intestine of a patient. As shown in FIG. 15V, administration of a dose at 1000 mg D1, 500 mg W2, W4, W6, W10, (i.e. 1000 mg at day 1, 500 mg at week 2, 500 mg at week 4, 500 mg at week 6, and 500 mg at week 10) of the anti-TL1A antibody A219 can cover up to about 75 fold over-production (fold increase) of TL1A in the intestine of a patient.

For comparison, a reference antibody that only binds to trimeric TL1A was tested in the whole-body PBPK model (reference antibody sequence, light chain SEQ ID NO: 382 and heavy chain SEQ ID NO: 383). As shown in FIG. 15W, such reference antibody failed to consistently reduce or consistently keep the free TL1A concentration in diseased tissue of a patient below the free TL1A concentration in the corresponding tissue of a normal healthy volunteer, when the diseased tissue overproduces TL1A by 50 fold or higher over the TL1A production in the corresponding tissue of a normal healthy volunteer. This is in sharp contrast with FIG. 15A, in which the anti-TL1A antibody A219 that binds to both monomeric TL1A and trimeric TL1A consistently reduced and kept free TL1A concentration in the diseased tissue below the free TL1A concentration in the corresponding tissue of a normal healthy volunteer, even if the diseased tissue overproduced TL1A by 100-fold over the TL1A production in the corresponding tissue of a normal healthy volunteer. As described above and shown in FIGS. 12C, 13C, and 14A-14B, the UC patients were determined to have a 50-fold overproduction of TL1A in the diseased tissue to recapitulate the observed modest increase in serum TL1A concentration. As such, an anti-TL1A antibody that binds to both monomeric and trimeric TL1A reduces the free TL1A concentration in the diseased tissue of a patient below the free TL1A concentration in the corresponding tissue of a normal healthy volunteer.

To further demonstrate the advantage of the anti-TL1A antibody that binds to both monomeric and trimeric TL1A in treating patients and reducing free TL1A concentration in diseased tissues, such antibodies were directly compared with a reference antibody that only binds to trimeric TL1A. As shown in FIGS. 15X-15Z, the anti-TL1A antibody A219 that binds to both monomeric and trimeric TL1A consistently and significantly reduced the free TL1A concentration in the diseased tissue below the free TL1A concentration in the diseased tissue resulted from the treatment of the reference anti-TL1A antibody that binds to only trimeric TL1A (reference antibody sequence, light chain SEQ ID NO: 382 and heavy chain SEQ ID NO: 383).

### Example 21: Population PK (popPK) Models for Determining the Effective Dose

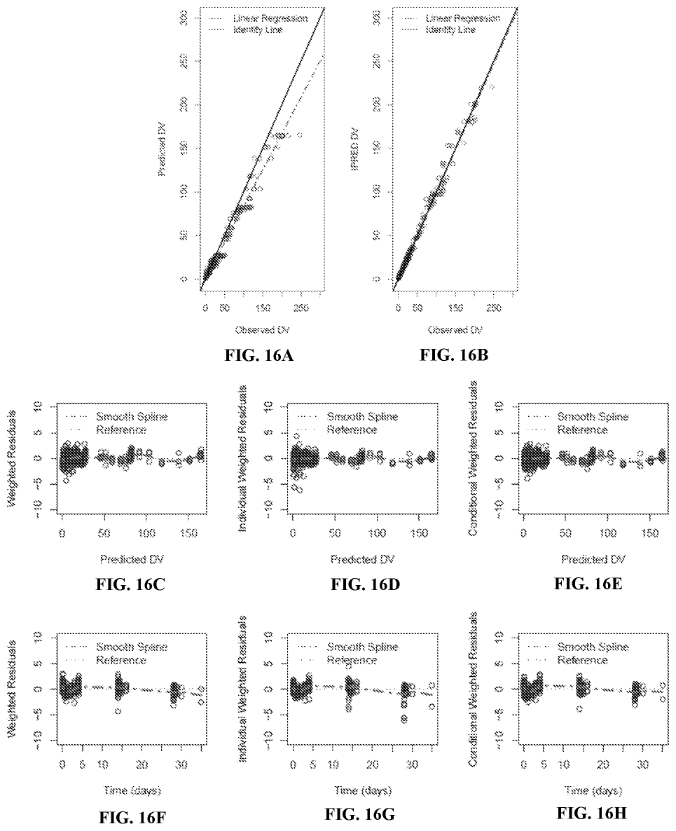

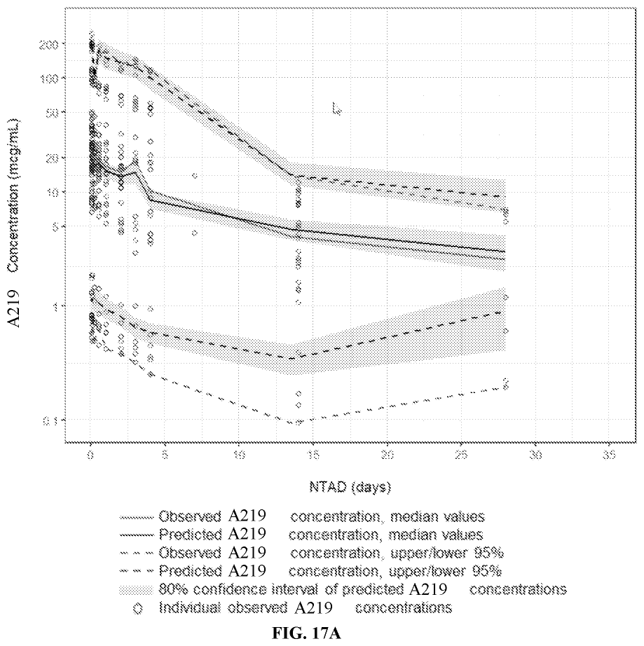

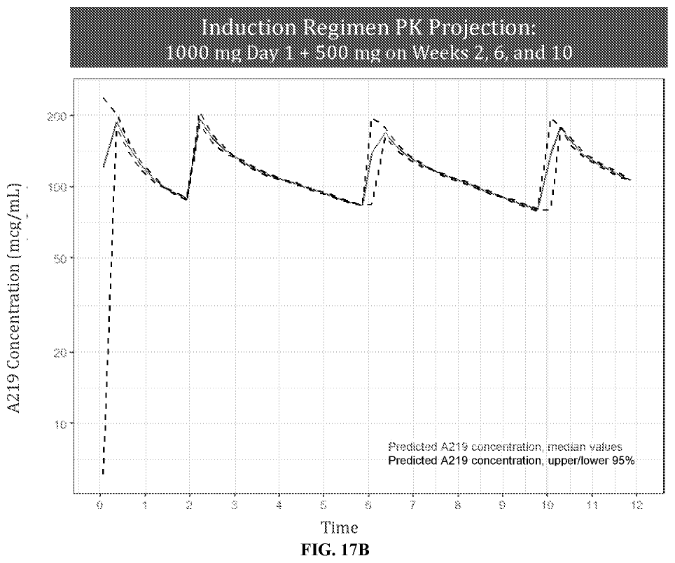

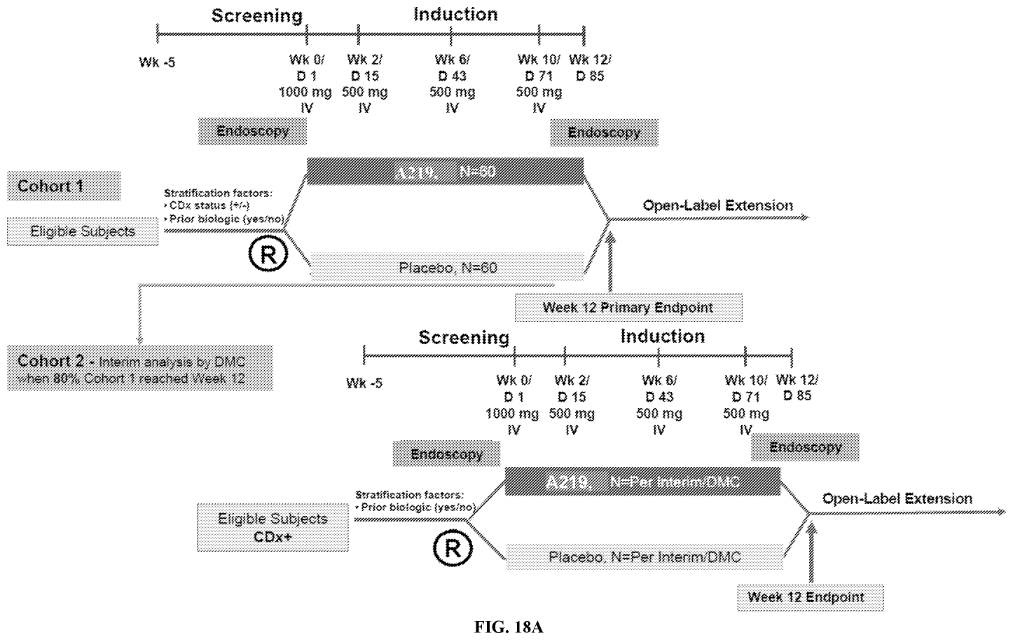

Additionally, based on the available PK data available from Example 16, a population PK model was built to accurately simulate and predict A219 PK in the population of normal healthy volunteers. The available PK data were best described by a 2-compartment model with linear elimination. Demographic variables (including sex, age, race, and body size related variables) and laboratory clinical variables (including hematological, urine, and chemical variables) were tested for inclusion in the model for effect on the clearance and the volume of distribution in the central compartment. None of these variables were identified as significant covariates on the 2 PK parameters evaluated. The population PK parameter estimates, and standard error (SE) are described in Table 27. Residual variability of A219 concentrations associated with the population PK model was 11.9%. Goodness of fit plots are presented in FIGS. 16A-16H. These plots indicated good correlations between population predicted A219 concentrations (“Predicted DV”) and observed A219 concentrations (“Observed DV”), and between individual predicted A219 concentrations (“IPRED DV”) and Observed DV. No bias in standardized weighted residuals versus predicted concentrations or versus time was noted. Evaluation of the visual predictive check (FIG. 17A) indicated that the population PK model could adequately predict the observed A219 concentrations and was suitable to be used to simulate A219 concentrations.

TABLE 27
PK Parameter Estimates of the Population PK Model of A219

| | | |
|---|---|---|
| Parameters | Estimates | SE (%) |

PK Parameters

| | | |
|---|---|---|
| CL (L/Day) | 0.156 | 15.2 |
| V1 (L) | 3.62 | 8.6 |
| V2 (L) | 2 | 0 |
| Q (L/Day) | 0.149 | 7.6 |

Inter-Individual Variability (% CV)

| | | |
|---|---|---|
| CL | 39.75 | 54.6 |
| V1 | 11.03 | 44.6 |
| V2 | 0 FIXED | — |
| Q | 0 FIXED | — |

Residual error

| | | |
|---|---|---|
| RV (%) | 11.9 | 8.2 |

Having established a popPK model, the popPK was used to select an induction dose to rapidly achieve steady state concentration. As shown in FIG. 17B, the loading dose of the induction regimen of 1000 mg at day 1, 500 mg at week 2, 500 mg at week 6, and 500 mg at week 10 ensures achievement of induction steady state concentration from day 1. Furthermore, as shown above in the whole-body PBPK, such an induction regimen can address over 100× over-production of TL1A in the colon within the first 5 weeks of induction and over 60× over-production for the 12 week period.

### Example 22: A Phase 2, Multi-Center, Double-Blind, Placebo-Controlled Study to Evaluate the Safety, Efficacy, and Pharmacokinetics of Induction Therapy with A219 in Subjects with Moderately to Severely Active Ulcerative Colitis

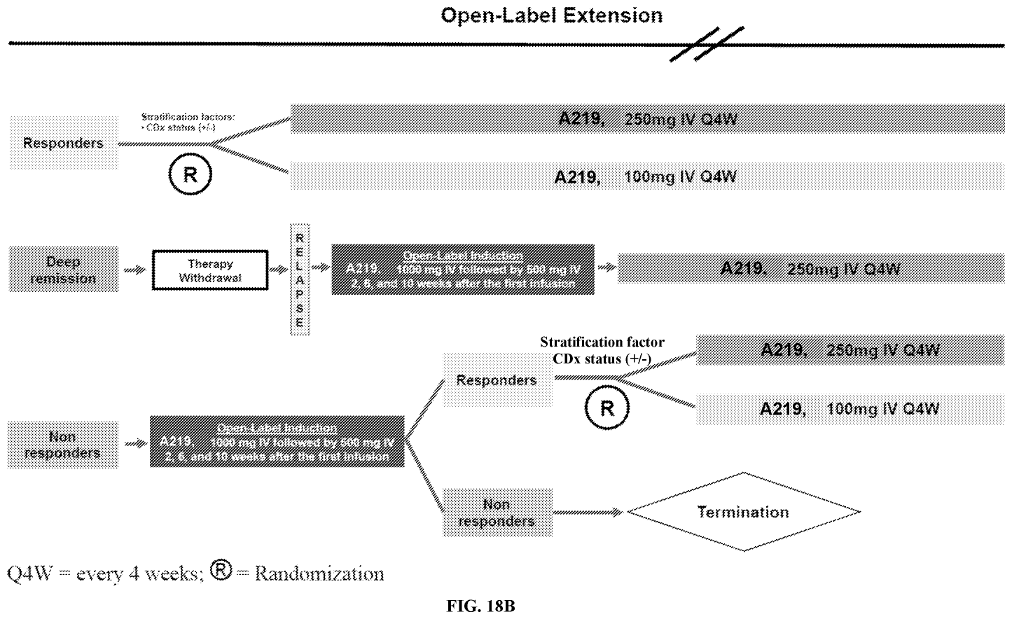

To validate the efficacy of the anti-TL1A antibody A219 in ulcerative colitis (UC), a phase 2 clinical trial is conducted. The clinical trial includes an induction period, as shown in FIG. 18A, and an open-label extension (maintenance) period, as shown in FIG. 18B. The detailed design of the clinical trial protocol is shown in the protocol synopsis of Table 28 below. The disclosure provides that clearance of free soluble TL1A (sTL1A) from the gut will translate into efficacy. Therefore, a physiologically based PK model (as described in Example 20) was used to predict the impact of various dosing regimen of A219 on the level of sTL1A in normal and disease states in the central compartment (serum) and gut. The model predicts that the proposed induction regimen will lead to sTL1A levels of lower than healthy volunteers if the over-production level of sTL1A in the colon is as high as 60-fold. After the 12-week induction, subjects who are in response will continue in the open-label extension randomized to 2 maintenance regimens. The maintenance regimen of 250 mg Q4W is selected to maintain the sTL1A level to below that of healthy volunteers if the production of sTL1A in the colon is up to 20× and the 100 mg Q4W regimen is selected to maintain the sTL1A level to below that of healthy volunteers if the production of sTL1A in the colon is up to 10×.

TABLE 28
PROTOCOL SYNOPSIS

| | |
|---|---|
| TITLE: | A Phase 2, Multi-Center, Double-Blind, Placebo-Controlled Study to Evaluate |
| | the Safety, Efficacy, and Pharmacokinetics of Induction Therapy with |
| | A219 in Subjects with Moderately to Severely Active Ulcerative Colitis |
| PROJECT PHASE: | Phase 2 |
| OBJECTIVE: | Primary: |
| | To assess the safety and tolerability of A219 following 12-weeks of |
| | induction therapy |
| | To compare the efficacy of A219 vs placebo for induction of clinical |
| | remission at Week 12 |
| | Secondary: |
| | All objectives below refer to comparison of A219-treated subjects vs |
| | placebo-treated subjects in Cohort 1. For the objectives where the |
| | companion diagnostic (CDx) status is a variable, a comparison of subjects |
| | in both Cohort 1 and Cohort 2 will be conducted. |
| | To compare the efficacy of A219 vs placebo for induction of |
| | endoscopic improvement at Week 12 |
| | To compare the efficacy of A219 vs placebo for induction of clinical |
| | response at Week 12 |
| | To compare the efficacy of A219 vs placebo in CDx positive |
| | (CDx+) subjects (Cohort 1 + Cohort 2) for induction of clinical |
| | remission at Week 12 |
| | To compare the efficacy of A219 vs placebo for induction of |
| | histologic remission at Week 12 |
| | To compare the efficacy of A219 vs placebo for induction of |
| | histologic-endoscopic mucosal improvement at Week 12 |
| | To compare the efficacy of A219 vs placebo in CDx+ subjects |
| | (Cohort 1 + Cohort 2) for induction of endoscopic improvement at |
| | Week 12 |
| | To compare the efficacy of A219 vs placebo in CDx+ subjects |
| | (Cohort 1 + Cohort 2) for induction of clinical response at Week 12 |
| | To compare the efficacy of A219 vs placebo in CDx+ subjects (Cohort |
| | 1 + Cohort 2) for induction of histologic remission at Week 12 |
| | To compare the efficacy of A219 vs placebo in CDx+ subjects |
| | (Cohort 1 + Cohort 2) for induction of histologic-endoscopic mucosal |
| | improvement at Week 12 |
| | To compare the efficacy of A219 in CDx+ (Cohort 1 + Cohort 2) vs |
| | CDx negative (CDx−) subjects for induction of clinical remission at Week |
| | 12 |
| | To compare the efficacy of A219 vs placebo for induction of |
| | mucosal healing at Week 12 |
| | To compare the efficacy of A219 vs placebo in CDx+ subjects |
| | (Cohort 1 + Cohort 2) for induction of mucosal healing at Week 12 |
| | To compare the efficacy of A219 vs placebo for change in |
| | Inflammatory Bowel Disease Questionnaire (IBDQ) at Week 12 |
| | To compare the efficacy of A219 vs placebo in CDx+ subjects |
| | (Cohort 1 + Cohort 2) for change in IBDQ at Week 12 |
| | To compare the efficacy of A219 vs. placebo in subjects who are |
| | CDx+ per alternative algorithm (Cohort 1 + Cohort 2) for clinical |
| | remission at Week 12 |
| | Exploratory: |
| | To assess the pharmacokinetics (PK) of A219 in subjects with |
| | ulcerative colitis (UC) over time |
| | To assess the effects of A219 on tissue and serum pharmacodynamic |
| | (PD) markers, including total TLIA concentrations over time |
| | To assess the effect of A219 on inflammatory biomarkers including |
| | fecal calprotectin and high sensitivity C-reactive protein (hsCRP) overtime |
| | To assess the proportion of subjects in 3-component Modified Mayo |
| | Score response, 3-component Modified Mayo Score remission, |
| | endoscopic improvement, Robarts histopathology index (RHI) histologic |
| | remission, Geboes score histologic remission, and mucosal healing at |
| | Week 50 |
| | To assess the change in Partial Mayo Score over time |
| | To assess the change in Geboes Index and RHI from Baseline to |
| | Week 12 and Week 50 |
| | To assess the exposure-response relationship of A219 on PD markers |
| | over time |
| | To assess the proportion of subjects achieving corticosteroid-free- |
| | remission at Week 50 |
| STUDY DESIGN: | This is a multi-center, double-blind, randomized, placebo-controlled proof |
| | of concept study designed to assess the safety, tolerability, and efficacy of |
| | A219 following 12 weeks of induction therapy in subjects with UC. This |
| | study will be conducted under the aegis of a Data Monitoring Committee |
| | (DMC) and will commence following the demonstration of an acceptable |
| | safety profile of A219 at a dose of ≥ 500 mg in the multiple ascending dose |
| | study in normal healthy volunteers (Example 16). |
| | The study has 4 periods (Screening Period, Induction Period [IP], Open- |
| | Label Extension [OLE] Period, and Follow-Up [FU] Period). The study will |
| | have 2 Cohorts that will enroll subjects in a sequential fashion utilizingan |
| | adaptive design as described below. |
| | Cohort 1: Following the Screening Period, approximately 120 eligible |
| | subjects with moderately to severely active UC will enter the IP and be |
| | randomized in a 1:1 fashion to receive intravenous (IV) administration of |
| | A219 1000 mg on Week 0/Day 1, followed by 500 mg on Weeks 2, 6, and |
| | 10, or placebo at the same timepoints. Randomization will be stratifiedby |
| | CDx status of positive (CDx+) or negative (CDx−) and prior biologic |
| | experience (yes/no) at Week 0/Day 1. Subjects who discontinue from the |
| | study will have a follow-up period of 12 weeks after last dose. |
| | Cohort 2: When approximately 80% of Cohort 1 subjects (i.e., ~96 subjects) |
| | have reached Week 12 or early terminated from the study, the DMC will |
| | conduct an unblinded analysis of clinical efficacy in CDx+ subjects and will |
| | recommend whether an expansion to Cohort 2 is warranted. The planned |
| | sample size for CDx+ subjects (combining Cohort 1 and Cohort 2) will be |
| | approximately 40, in the case where Cohort 2 is initiated. For Cohort 2, |
| | eligible subjects (who must be CDx+) will enter the IP and be randomized |
| | in a 1: 1 fashion to receive IV administration of A219 1000 mg on |
| | Week 0/Day 1, followed by 500 mg on Weeks 2, 6, and 10, or placebo at |
| | the same timepoints. Randomization will be stratified by prior biologic |
| | experience (yes/no) at Baseline. Subjects who discontinue from the study |
| | will have a follow-up period of 12 weeks after last dose. |
| | Subjects who complete the 12-week IP from either Cohort will have the |
| | option to enter OLE. During OLE, starting at Week 14 visit: |
| | Subjects who have achieved deep remission (defined as in clinical |
| | remission [endoscopic subscore of 0 or 1, rectal bleeding subscore of 0, and |
| | stool frequency subscore of 0 or 1 and not greater than Baseline] and RHI < |
| | 3) will continue in the study and have therapy withdrawal. |
| | Upon disease relapse (defined as rectal bleeding score of ≥1, and either |
| | hsCRP ≥ 5 and/or fecal calprotectin ≥ 250), subjects can receive another |
| | course of induction therapy (1000 mg IV followed by 500 mg IV 2, 6, and |
| | 10 weeks after the first infusion) followed by maintenance therapy of 250 |
| | mg IV every 4 weeks (Q4W) for atotal of 50 weeks. |
| | Responders (defined as reduction from Baseline ≥ 2 points and ≥ 30% |
| | in 3-component Modified Mayo Score, accompanied by a reduction ≥ 1in |
| | rectal bleeding subscore or absolute rectal bleeding subscore ≤ 1) who have |
| | not achieved deep remission will be re-randomized, stratifiedby CDx status |
| | of CDx+ or CDx−, to either 250 mg IV Q4W or 100 mg IV Q4W, starting at |
| | Week 14 until Week 50. |
| | Nonresponders will receive an open-label induction regimen of |
| | 1000 mg of A219 on Week 14, followed by 500 mg on Weeks 16,20, and |
| | 24. Nonresponders who do not respond at Week 28 (per investigator |
| | discretion) should be discontinued from the study. |
| | Nonresponders who respond at Week 28 (per investigator discretion) |
| | will be re-randomized to either 250 mg IV Q4W or 100 mg IV Q4W, |
| | starting at Week 28 for a total of 50 weeks. |
| | The study may be amended by the Sponsor to extend the OLE period |
| | beyond 50 weeks based on emerging safety data. |
| | The study also includes an optional PK sub-study during the IP for subjects |
| | who consent to additional PK sampling. |
| SAMPLE SIZE: | The study is planned to randomize up to approximately 170 subjects, |
| | approximately 120 in Cohort 1 and up to 50 in Cohort 2. A sample size of |
| | 60 per arm in Cohort 1 will enable a statistical power of >80% for the |
| | primary endpoint at 1-sided significance level of 0.025 using Cochran- |
| | Mantel-Haenszel (CMH), assuming clinical remission rate of 5% for |
| | placebo and 24% for A219. Additionally, the sample size will confer >80% |
| | power to achieve statistical significance for the 1^{st} secondary endpoint |
| | of endoscopic improvement with an overall 1-sided alpha level of |
| | 0.025, assuming the endoscopic improvement rates are 15% and 38% for |
| | placebo and A219 groups, respectively. |
| | Additionally, for analyses of the CDx population (combining CDx+ |
| | subjects from Cohort 1 and Cohort 2), a sample size of 40 per arm will |
| | provide a statistical power of ≥80% at a 1-sided alpha level of 0.025, |
| | according to a group sequential design with a non-binding futility interim |
| | analysis when 18 subjects per arm reach Week 12, assuming clinical |
| | remission rate of 5% for placebo and 31% for A219. |
| SUBJECT TYPE: | Male or female subjects ≥ 18 years of age with moderately to severely |
| | active UC. |
| FORMULATIONS: | A219 will be supplied in 10 ml vials each containing 500 mg A219(60 |
| | mg/mL solution) for IV administration after reconstitution. |
| DOSAGE: | Subjects will be stratified by CDx+/CDx− status and prior biologic |
| | experience (yes/no) in a 1:1 ratio to: |
| | A219 1000 mg IV on Week 0/Day 1, followed by 500 mg IV on |
| | Weeks 2, 6, and 10 |
| | Placebo IV on Week 0/Day 1, followed by placebo IV on Weeks 2, 6, |
| | and 10 |
| | During OLE: |
| | Deep remitters will enter the therapy withdrawal period o All subjects who develop disease relapse after therapy withdrawal will |
| | receive another course of induction therapy (1000 mg IV followed by 500 |
| | mg IV 2, 6, and 10 weeks after the first infusion) followed by maintenance |
| | therapy of 250 mg IV Q4W for a total of 50 weeks. |
| | Responders at the end of Week 12 will be stratified by CDx status of |
| | CDx+ or CDx− and re-randomized to receive one of the following |
| | regimens: |
| | A219 250 mg IV on Week 14 then Q4W until Week 50 A219 |
| | 100 mg IV on Week 14 then Q4W until Week 50 |
| | Nonresponders at the end of Week 12 will receive open-label A219 |
| | 1000 mg IV on Week 14, followed by A219 500 mg IV on Weeks 16, 20, |
| | and 24. Subjects who do not respond by Week 26 should be discontinued |
| | from the study. Subjects who respond by Week 26 will bestratified by CDx |
| | status of CDx+ or CDx− and re-randomized to receiveone of the following |
| | regimens: A219 |
| | 250 mg IV on Week 28 then Q4W for a total of 50 weeks A219 |
| | 100 mg IV on Week 28 then Q4W for a total of 50 weeks |
| ROUTE OF | All study drug will be reconstituted in 250 mL of 0.9% normal saline (NS) |
| ADMINISTRATION: | and will be administered IV over 30 minutes. |
| STUDY | Primary endpoints: |
| ENDPOINTS: | The proportion of subjects reporting adverse events (AEs), serious |
| | adverse events (SAEs), AEs leading to discontinuation, and markedly |
| | abnormal laboratory values. |
| | The proportion of subjects in the 3-component Modified Mayo Score |
| | clinical remission (as defined by endoscopic subscore of 0 or 1, rectal |
| | bleeding subscore of 0, and stool frequency subscore of 0 or 1 and not |
| | greater than Baseline) at Week 12. The 3-component Modified Mayo Score |
| | ranges from 0-9 and includes rectal bleeding, stool frequency and |
| | endoscopic assessment domains. |
| | Secondary endpoints: |
| | The proportion of subjects with endoscopic improvement, as defined by |
| | endoscopy subscore ≤ 1 with no friability) at Week 12. |
| | The proportion of subjects in 3-component Modified Mayo Score clinical |
| | response at Week 12. The 3-component Modified Mayo Score clinical |
| | response is defined by reduction from Baseline ≥ 2 points and ≥30% in3- |
| | component Modified Mayo Score, accompanied by a reduction ≥ 1 inrectal |
| | bleeding subscore or absolute rectal bleeding subscore ≤ 1. |
| | The proportion of subjects in the 3-component Modified Mayo Score |
| | clinical remission (as defined by endoscopic subscore of 0 or 1, rectal |
| | bleeding subscore of 0, and stool frequency subscore of 0 or 1 and not greater |
| | than Baseline), in CDx+ subjects (Cohort 1 + Cohort 2) treated with A219 |
| | compared to CDx+ placebo-treated subjects at Week 12. The 3-component |
| | Modified Mayo Score ranges from 0-9 and includes rectal bleeding, stool |
| | frequency, and endoscopic assessment domains. |
| | The proportion of subjects with histologic remission (defined Geboes |
| | score ≤ 3.1) at Week 12. |
| | The proportion of subjects with histologic-endoscopic mucosal |
| | improvement (defined as Geboes score ≤ 3.1 and endoscopy subscore ≤ |
| | 1 with no friability) at Week 12. |
| | The proportion of subjects with endoscopic improvement, as defined by |
| | endoscopy subscore ≤ 1 with no friability, in CDx+ subjects (Cohort 1 + |
| | Cohort 2) treated with A219 compared to CDx+ placebo-treatedsubjects |
| | at Week 12. |
| | The proportion of subjects in 3-component Modified Mayo Score |
| | clinical response in CDx+ subjects treated with A219 compared to CDx+ |
| | placebo-treated subjects at Week 12. The 3-component Modified Mayo |
| | Score clinical response is defined by reduction from Baseline ≥ 2points |
| | and ≥30% in 3-component Modified Mayo Score, accompanied by a reduction ≥ 1 |
| | in rectal bleeding subscore or absolute rectal bleeding subscore ≤ 1. |
| | The proportion of subjects with histologic remission, defined as Geboes |
| | score ≤ 3.1, in CDx+ subjects (Cohort 1 + Cohort 2) treated with A219 |
| | compared to CDx+ placebo-treated subjects at Week 12. |
| | The proportion of subjects with histologic-endoscopic mucosal |
| | improvement (defined as Geboes score ≤ 3.1 and endoscopy subscore ≤ |
| | 1 with no friability), in CDx+ subjects (Cohort 1 + Cohort 2) treated with |
| | A219 compared to CDx+ placebo-treated subjects at Week 12. |
| | The proportion of subjects with clinical remission (defined as |
| | endoscopic subscore of 0 or 1, rectal bleeding subscore of 0, and stool |
| | frequency subscore of 0 or 1 and not greater than Baseline) in CDx+ |
| | subjects (Cohort 1 + Cohort 2) treated with A219 compared to in CDx− |
| | subjects treated with A219 at Week 12. |
| | The proportion of subjects with mucosal healing (defined as Geboes |
| | score ≤ 2B. 1 and endoscopy subscore of ≤ 1) at Week 12. |
| | The proportion of subjects with mucosal healing (defined as Geboes |
| | score ≤ 2B.1 and endoscopy subscore of ≤ 1), in CDx+ subjects (Cohort |
| | 1 + Cohort 2) treated with A219 compared to CDx+ placebo-treated |
| | subjects at Week 12. |
| | The proportion of subjects with IBDQ response, as defined by ≥16- |
| | point increase from Baseline at Week 12. |
| | The proportion of subjects with IBDQ response, as defined by ≥16- |
| | point increase from Baseline, in CDx+ subjects (Cohort 1 + Cohort 2) |
| | treated with A219 compared to CDx+ placebo-treated subjects at Week |
| | 12. |
| | The proportion of subjects in the 3-component Modified Mayo Score |
| | clinical remission (as defined by endoscopic subscore of 0 or 1, rectal |
| | bleeding subscore of 0, and stool frequency subscore of 0 or 1 and not |
| | greater than Baseline), in CDx+ subjects per alternative algorithm (Cohort |
| | 1 + Cohort 2) treated with A219 compared to CDx+ |
| | placebo-treated subjects per alternative algorithm at Week 12. The 3- |
| | component Modified Mayo Score ranges from 0-9 and includes rectal |
| | bleeding, stool frequency, and endoscopic assessment domains. |
| | Exploratory endpoints: |
| | The pharmacokinetics of A219 in subjects with UC after multiple |
| | doses |
| | The change from Baseline in serum and fecal inflammatory biomarkers |
| | (PD markers) |
| | The proportion of subjects in 3-component Modified Mayo Score |
| | response, 3-component Modified Mayo Score remission, endoscopic |
| | improvement, RHI histologic remission, Geboes score histologic |
| | remission, and mucosal healing at Week 50 |
| | The change in Partial Mayo Score (with or without PGA component) |
| | over time |
| | The change in Geboes Index and RHI from Baseline to Week 12 and |
| | Week 50 |
| | The exposure-response relationship of A219 on PD markers |
| | Within subpopulation of subjects on corticosteroid at study entry, the |
| | proportion of subjects in clinical remission and off of corticosteroid at |
| | Week 50 |
| INCLUSION | Subjects are required to meet the following criteria in order to be included in |
| CRITERIA: | the study: |
| | 1. Male or female ≥ 18 years of age. |
| | 2. Subjects must have had a documented diagnosis of UC (endoscopy + |
| | histology) to be eligible for study participation. For subjects with no |
| | documented confirmation of UC diagnosis or if previous diagnosis isnot |
| | deemed conclusive, UC diagnosis must be confirmed at time of screening |
| | colonoscopy. |
| | 3. Moderately to severely active UC as defined by 3-component Modified |
| | Mayo score (3 components of rectal bleeding, stool frequency, and |
| | endoscopy) of 4 to 9, inclusive, with Modified Mayo endoscopic subscore ≥ |
| | 2 and rectal bleeding subscore ≥ 1. |
| | 4. Subjects must satisfy at least one of the following criteria: |
| | a) In the past, had an inadequate response to one or more of the |
| | following treatments: |
| | Oral prednisone ≥ 40 mg/day (or equivalent) or budesonide ≥ 9 |
| | mg/day or equivalent or beclomethasone ≥ 5 mg/day for at least2 weeks |
| | Corticosteroid dependence as defined by failed to successfully |
| | taper to < 10 mg/day of prednisone or equivalent (i.e., had a |
| | flare of disease) within 3 months of starting therapy, or if relapse |
| | occurs within 3 months of stopping corticosteroids |
| | Immunosuppressants (azathioprine ≥ 2 mg/kg/day or |
| | 6-mercaptopurine ≥ 1.0 mg/kg/day [or documentation of a therapeutic |
| | concentration of 6-thioguanine nucleotide]) for atleast 8 weeks |
| | An approved anti-TNF agent at an approved labeled dose for at |
| | least 8 weeks |
| | Vedolizumab at the approved labelled dose for at least 8 weeks |
| | An approved JAK inhibitor (e.g., tofacitinib) at an approved |
| | labelled dose for at least 8 weeks |
| | An approved anti-IL-12/23 (e.g., ustekinumab) at an approved |
| | labelled dose for at least 8 weeks |
| | An approved sphingosine 1-phosphate receptor (S1PR) |
| | modulator at an approved labelled dose for least 12 weeks |
| | OR |
| | b) Had been intolerant to one or more of the above-mentioned |
| | treatments (e.g., unable to achieve doses or treatment durations because |
| | of dose limiting side effects [e.g., leukopenia, psychosis, uncontrolled |
| | diabetes, elevated liver enzymes]). |
| | OR |
| | c) Currently receiving one or more of the following treatments: |
| | Oral Prednisone ≥ 10 mg/day (or equivalent) for at least 3 months |
| | Immunosuppressants [azathioprine ≥ 2 mg/kg/day or |
| | 6-mercaptopurine ≥ 1.0 mg/kg/day (or documentation of a therapeutic |
| | concentration of 6-thioguanine nucleotide)] for atleast 8 weeks. |
| | Notes on subjects who have had prior biologic/biologic-like therapy(ies) |
| | (anti-TNF, JAK inhibitor, S1PR modulator, anti-IL-12/23, and/or |
| | vedolizumab): |
| | The study will include a maximum of 70% subjects who have had |
| | prior biologic/biologic-like therapy(ies) experience. Upon reachingthe |
| | maximum number of allowed biologic/biologic-like experienced subjects |
| | (70%), subjects who have had prior biologic/biologic-like experience will |
| | no longer be allowed to enterthe study. |
| | Subject cannot have failed (no response, insufficient response, loss |
| | of response, and/or intolerance) >3 classes or >4 individual |
| | biologic/biologic-like therapies (refer to exclusion criterion #26). |
| | 5. For subjects who are women of childbearing potential (WOCBP) |
| | involved in any sexual intercourse that could lead to pregnancy, the |
| | subject has used two highly effective methods of contraception for atleast |
| | 4 weeks prior to Day 1 and agrees to continue to use two highly |
| | effective methods of contraception until at least 12 weeks after the lastdose |
| | of study drug. |
| | 6. Male subjects must use, with their female partner of childbearing |
| | potential, two highly effective methods of contraception and refrain from |
| | sperm donation from screening to 12 weeks after the last dose ofstudy |
| | drug. |
| | 7. Subject must meet drug stabilization requirements, as applicable: |
| | a) Oral corticosteroid treatment must be equivalent of ≤20 mg |
| | prednisone or ≤9 mg budesonide or beclomethasone ≤ 5 mg daily at a |
| | stable dose for at least 2 weeks prior to randomization |
| | b) Oral aminosalicylates should be at a stable dose for at least 2 weeks |
| | prior to randomization |
| | c) Azathioprine and 6-mercaptopurine should be at a stable dose for at |
| | least 4 weeks prior to randomization |
| | 8. Able to provide written informed consent and understand and comply |
| | with the requirements of the study. |
| | 9. For Cohort 2 only: Subjects must be CDx+. |
| EXCLUSION | Subjects with the following characteristics will be excluded from the study: |
| CRITERIA: | Sex and Reproductive Status |
| | 1. WOCBP and men with female partners of childbearing potential who |
| | are unwilling or unable to use two highly effective methods of |
| | contraception to avoid pregnancy for the entire study period and for upto |
| | 12 weeks after the last dose of study drug. |
| | 2. Women who are pregnant or breastfeeding. |
| | 3. Women with a positive pregnancy test on enrollment or prior to |
| | randomization. |
| | Target Disease Exceptions |
| | 14. Diagnosis of Crohn's disease or indeterminate colitis. |
| | 5. UC limited to the rectum (<15 cm from anal verge). |
| | 6. Current evidence of fulminant colitis, toxic megacolon, or bowel |
| | perforation. |
| | 7. Current or impending need for colostomy or ileostomy. |
| | 8. Previous total proctocolectomy or partial colectomy. |
| | 9. Surgical bowel resection within 3 months before screening. |
| | 10. Concomitant primary sclerosing cholangitis (PSC) |
| | Medical History and Concurrent Diseases |
| | 11. Past or current evidence of definite low-grade or high-grade colonic |
| | dysplasia that has not been completely removed. |
| | 12. Subjects who are scheduled or anticipate the need for surgery, aside |
| | from dermatologic procedures. |
| | 13. Subjects who have a history of clinically significant drug or alcohol |
| | abuse. |
| | 14. Concomitant illness that in the opinion of the Investigator, is likely to |
| | require systemic glucocorticosteroid therapy during the study (e.g., |
| | moderate to severe asthma). |
| | 15. Current symptoms of severe, progressive, or uncontrolled renal, |
| | hepatic, hematological, pulmonary, cardiac, neurological, |
| | ophthalmologic or cerebral disease. Concomitant medical conditionsthat |
| | in the opinion of the Investigator might place the subject at unacceptable |
| | risk for participation in this study. |
| | 16. Subjects with a history of cancer within the last 5 years (other than |
| | non-melanoma skin cell cancers cured by local resection). Existing non- |
| | melanoma skin cell cancers must be removed prior to enrollment. Subjects |
| | with carcinoma in situ or localized cervical cancer, treated with definitive |
| | surgical intervention, are allowed. |
| | 17. Subjects at risk for tuberculosis (TB). Specifically, subjects with: |
| | a) A history of active TB |
| | b) Current clinical, radiographic or laboratory evidence of active TB |
| | c) Latent TB which was not successfully treated. Subjects with a |
| | positive TB screening test indicative of latent TB will not be eligible for the |
| | study unless active TB infection has been ruled out, and an appropriate |
| | course of intervention for latent TB has been initiated at least 2 weeks prior |
| | to randomization, and no evidence of active TB on chest x-ray during |
| | Screening. |
| | 18. Subjects with any serious bacterial infection within the last 3 months, |
| | unless treated and resolved with antibiotics, or any chronic bacterial |
| | infection (such as chronic pyelonephritis, osteomyelitis and |
| | bronchiectasis). |
| | 19. Female subjects who have had a breast cancer screening that is |
| | suspicious for malignancy, and in whom the possibility of malignancy |
| | cannot be reasonably excluded following additional clinical, laboratory or |
| | other diagnostic evaluations. |
| | 20. Subjects with any active infections (excluding fungal infections of nail |
| | beds) including, but not limited to, those that require intravenous (IV) |
| | antimicrobial treatment 4 weeks or oral antimicrobial treatment 2 weeks |
| | prior to randomization. Subjects with evidence of Human |
| | Immunodeficiency Virus (HIV), Hepatitis B or Hepatitis C infection |
| | detected during screening are also excluded, but subjects with successfully |
| | treated Hepatitis C with no recurrence for ≥1 year are allowed. Subjects |
| | with active documented or suspected COVID-19 |
| | infection within 4 weeks of randomization or asymptomatic SARS-COV- |
| | 2 PCR test within 2 weeks of randomization are excluded. |
| | 21. Subjects with herpes zoster reactivation or cytomegalovirus (CMV) |
| | that resolved less than 2 months prior to signing informed consent. |
| | 22. Subjects who have received any live vaccines within 3 months of the |
| | anticipated first dose of study medication or who will have need of alive |
| | vaccine at any time during the study. |
| | Physical and Laboratory Test Findings |
| | 23. Positive stool Polymerase Chain Reaction (PCR) or culture for enteric |
| | pathogens. |
| | 24. Stool positive for Clostridium difficile (C. difficile) toxin. Subjects who |
| | are positive can be retested after the completion of a full course of |
| | treatment for C. difficile infection. |
| | 25. Any of the following lab values: |
| | a) Hemoglobin (Hgb) < 8.0 g/dL (80 g/L) |
| | b) White blood cell (WBC) < 2,500/mm³ (2.5 × 10⁹/L) |
| | c) Neutrophils < 1,000/mm³ (1 × 10⁹/L) |
| | d) Platelets < 100,000/mm³ (100 × 10⁹/L) |
| | e) Serum creatinine > 2 times upper limit of normal (ULN) |
| | f) Serum alanine aminotransferase (ALT) or aspartate |
| | aminotransferase (AST) >2 times ULN |
| | g) Any other laboratory test results that, in the opinion of the |
| | Investigator, might place the subject at unacceptable risk for |
| | participation in this study |
| | Prohibited Therapies and/or Medications |
| | 26. Failed (no response, insufficient response, loss of response, and/or |
| | intolerance) >3 classes (anti-TNF, anti-integrin, anti-IL12/23, JAK |
| | inhibitor, SIPR modulator) or >4 individual biologic/biologic-like |
| | therapies. |
| | 27. Any marketed biologic or biologic-like within 2 weeks for tofacitinib, 8 |
| | weeks for anti-TNF agents, 10 weeks for SIPR modulators, and 12 weeks |
| | for vedolizumab and ustekinumab prior to randomization or if drug level per |
| | therapeutic dose monitoring is greater than lower limit of detection. |
| | 28. Any biologic immunomodulators not covered in exclusion criterion 27, |
| | used for UC or other conditions within 8 weeks or 5 half-lives, whichever is |
| | longer, prior to randomization or if drug level per therapeutic dose |
| | monitoring is greater than lower limit of detection. |
| | 29. Rituximab within 1 year prior to randomization. |
| | 30. Parenteral corticosteroids within 4 weeks or rectal administration of |
| | corticosteroids within 2 weeks prior to randomization. |
| | 31. Rectal administration of 5-ASA within 2 weeks prior to randomization. |
| | 32. Tacrolimus, methotrexate, cyclosporine, mycophenolate mofetil |
| | (CellCept ®), immunoadsorption columns (such as Prosorba columns), D |
| | Penicillamine, Leflunomide, Thalidomide, fish-oil preparations, probiotics, |
| | fecal transplantation, non-steroidal anti-inflammatory agents(NSAIDs), |
| | aspirin > 81 mg/day within 2 weeks prior to randomization. |
| | 33. Other investigational chemical agent within 30 days or other |
| | investigational biologic agent within 8 weeks or 5 half-lives (whicheveris |
| | longer) of randomization. |
| | 34. Prior exposure to A219. |
| | Other Exclusion Criteria |
| | 35. Prisoners or subjects who are compulsorily detained (involuntarily |
| | incarcerated) for treatment of either a psychiatric or physical (e.g., |
| | infectious disease) illness must not be enrolled into this study. |
| | 36. Legal or mental incapacitation, or inability to understand and comply |
| | with the requirements of the study. |
| Statistical | Statistical methods will be detailed in the Statistical Analysis Plan. The |
| Methods: | SAP will provide details about the method of analysis and specific planned |
| | analyses, and will be prepared and approved by Prometheus Biosciences |
| | and its designees before study database lock and unblinding of subject |
| | treatment assignments. |
| | The analysis populations are defined as follows: |
| | Full analysis set (FAS) from Cohort 1: all subjects randomized and |
| | treated in Cohort 1 |
| | FAS for CDx+: all CDx+ subjects who are randomized and treated in |
| | both Cohort 1 and Cohort 2 |
| | Safety analysis set: all subjects treated |
| | The following analyses will be performed: |
| | Efficacy: |
| | The efficacy assessment will test for the difference between A219 and |
| | placebo groups in FAS. |
| | The primary endpoint will be analyzed and compared between A219 and |
| | placebo treatment groups in FAS from Cohort 1. The primary endpoint, the |
| | proportion of subjects achieving clinical remission, will be tested between |
| | the 2 treatment groups at 1-sided significance level of 0.025 using CMH |
| | with stratification factors at randomization. If significant, the 1st secondary |
| | endpoint of proportion of subjects achieving endoscopic improvement will |
| | be tested between the 2 treatment groups at 1-sided significance level of |
| | 0.025. If significant, the 2nd, 3rd, 4th, and 5th secondary endpoints will be |
| | tested sequentially, each at 1-sided significance level of 0.025. Testing for |
| | statistical significance will stop when the first endpoint is not statistically |
| | significant at level of 0.025 and all remaining p values will be nominal. |
| | Treatment difference for primary and secondary endpoints for Cohort 1 will |
| | be estimated along with 95% CI for all subjects in FAS. The secondary |
| | endpoints in CDx+ subjects will be summarized and compared between |
| | A219 and placebo groups in FAS for CDx+, while the treatment difference |
| | will be estimated with 95% CI. Additional efficacy analysis will be detailed |
| | in SAP. |
| | Interim Efficacy Analysis: |
| | An interim analysis will be carried out when approximately 80% of |
| | subjects (approximately 96 subjects) in Cohort 1 have reached Week 12 or |
| | early terminated from the study. The DMC will review the unblinded |
| | efficacy and safety data and recommend on the expansion to Cohort 2. |
| | Decision rules to initiate Cohort 2 are determined according to the futility |
| | bounds of group sequential design of a sample size of 40 per arm with one |
| | interim analysis at the information fraction of 45%. Because the exact |
| | bounds will be calculated using the actual number of subjects with CDx+ |
| | included in the interim analysis, the final decision rules, along with |
| | sensitivity analysis, will be specified in the DMC SAP, prior to the interim |
| | analysis. |
| | Adverse Events: |
| | AEs will be coded using the most current version of Medical Dictionary for |
| | Regulatory Activities (MedDRA ®). |
| | A by-subject AE data listing, including verbatim term, preferred term (PT), |
| | system organ class (SOC), treatment, severity, seriousness criteria, |
| | relationship to drug, and action taken, will be provided. |
| | The number of subjects experiencing treatment-emergent adverse events |
| | (TEAEs) and number of TEAEs will be summarized by treatment using |
| | frequency counts for Safety analysis set. |
| | Medical History, chest x-ray, electrocardiogram (ECG), and physical |
| | examination will be listed by subject. |
| | Changes in ECGs and physical examinations will be described in the text of |
| | the final report. |
| | Concomitant Medications: |
| | Concomitant medications will be coded using the most current World |
| | Health Organization (WHO) drug dictionary and listed by treatment. |
| | Pharmacokinetics: |
| | Summary statistics of A219 concentrations and anti-drug antibody |
| | (ADA) by visit and by CDx+ and CDx−. |

### Example 23: A Phase 2a, Multi-Center, Open-Label Study to Evaluate the Safety, Efficacy, and Pharmacokinetics of A219 in Subjects with Moderately to Severely Active Crohn's Disease

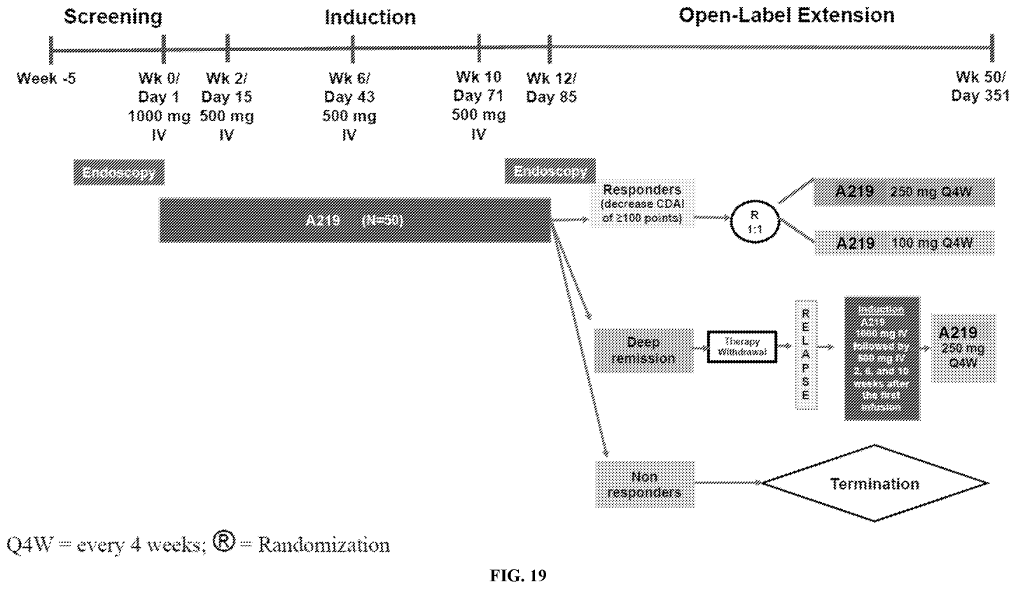

To validate the efficacy of the anti-TL1A antibody A219 in Crohn's disease (CD), a phase 2 clinical trial is conducted. The clinical trial includes an induction period, as shown in FIG. 19, and an open-label extension (maintenance) period, also shown in FIG. 19. The detailed design of the clinical trial protocol is shown in the protocol synopsis in Table 29 below. The disclosure provides that clearance of free soluble TL1A (sTL1A) from the gut will translate into efficacy. Therefore, a physiologically based PK model (as described in Example 20) was used to predict the impact of various dosing regimen of A219 on the level of sTL1A in normal and disease states in the central compartment (serum) and gut. The model predicts that the proposed induction regimen will lead to sTL1A levels of lower than healthy volunteers if the over-production level of sTL1A in the colon is as high as 60-fold. After the 12-week induction, subjects who are in response will continue in the open-label extension randomized to 2 maintenance regimens. The maintenance regimen of 250 mg Q4W is selected to maintain the sTL1A level to below that of healthy volunteers if the production of sTL1A in the colon is up to 20× and the 100 mg Q4W regimen is selected to maintain the sTL1A level to below that of healthy volunteers if the production of sTL1A in the colon is up to 10×.

TABLE 29
PROTOCOL SYNOPSIS

| | |
|---|---|
| TITLE: | A Phase 2a, Multi-Center, Open-Label Study to Evaluate the Safety, |
| | Efficacy, and Pharmacokinetics of A219 in Subjects with Moderately to |
| | Severely Active Crohn's Disease |
| PROJECT PHASE: | Phase 2a |
| OBJECTIVE: | Primary: |
| | To evaluate the safety and tolerability of A219 following 12-weeksof |
| | induction therapy |
| | To assess the proportion of subjects with endoscopic improvement |
| | (decrease in simple endoscopy score for Crohn's disease [SES-CD] ≥50% |
| | from Baseline) at Week 12 |
| | Secondary: |
| | To assess the proportion of subjects with clinical remission (Crohn's |
| | disease activity index [CDAI] <150) at Week 12 |
| | To assess the proportion of subjects with endoscopy and clinical |
| | improvement (decrease in SES-CD ≥50% AND reduction in CDAI ≥100 |
| | points from Baseline) at Week 12 |
| | To assess the proportion of subjects with biomarker and clinical |
| | improvement (decrease in high sensitivity C-reactive protein [hsCRP] or |
| | fecal calprotectin ≥50% from Baseline, among subjects with at least one |
| | elevated biomarker at Baseline, AND reduction in CDAI ≥100 points from |
| | Baseline) at Week 12 |
| | To assess the proportion of subjects with normalization of C-reactive |
| | protein (hsCRP < upper limit of normal [ULN]), among subjects with |
| | elevated concentrations at Baseline, at Week 12 |
| | To assess the proportion of subjects with normalization of fecal |
| | calprotectin (fecal calprotectin < ULN), among subjects with elevated |
| | concentrations at Baseline, at Week 12 |
| | To assess the proportion of subjects with clinical improvement |
| | (reduction in CDAI ≥100 points from Baseline) at Week 12 |
| | To assess the proportion of subjects with two component patient- |
| | reported outcome (PRO-2) remission (average daily abdominal pain score ≤1 |
| | point and average daily stool frequency ≤3 points with abdominal pain |
| | and stool frequency no worse than Baseline at Week 12) |
| | To assess the change in SES-CD score from Baseline to Week 12 |
| | To assess the pharmacokinetics (PK) of A219 |
| | To assess the immunogenicity of A219 |
| | Exploratory: |
| | To assess the change in CDAI and component scores over time |
| | To assess the effects of A219 on tissue and serum pharmacodynamic |
| | (PD) markers, including tumor necrosis factor-like cytokine 1A (TL1A) |
| | concentrations, endoscopic healing index (EHI), fecal calprotectin, and |
| | hsCRP in all subjects over time |
| | To assess the change in SES-CD at Week 50 from Baseline |
| | To characterize the change in Perianal Disease Activity Index (PDAI) |
| | score from Baseline to Week 12, Week 28, and Week 50 |
| | To characterize the effect of A219 for improvement and remission of |
| | enterocutaneous and/or perianal fistula during the Induction Period(IP) and |
| | Open-Label Extension (OLE) |
| | To assess all secondary endpoints at Week 50 |
| | To assess the change in global histological activity score (GHAS) and |
| | Robarts histopathology index (RHI) from Baseline to Week 12 and Week 50 |
| | To assess the proportion of subjects with histologic response and |
| | histologic remission at Week 12 and Week 50 |
| | To assess change in PRO-2 over time |
| | To assess change in extraintestinal manifestations over time |
| STUDY DESIGN: | This is a multi-center, open-label, proof of concept study designed to assess |
| | the safety, tolerability, and preliminary efficacy of A219 following |
| | 12 weeks of induction therapy in subjects with Crohn's disease (CD). This |
| | study will be conducted under the aegis of a Data Monitoring Committee |
| | (DMC) and will commence following the demonstration of an acceptable |
| | safety profile of A219 at a dose of ≥500 mg in the multiple ascending dose |
| | study in normal healthy volunteers (Study PR200-101). |
| | The study has 4 periods (Screening, IP, OLE and Follow-Up [FU] Period). |
| | Following the Screening Period, approximately 50 eligible subjects with |
| | moderately to severely active CD will enter the IP to receive A219 1000 mg |
| | on Week 0/Day 1, followed by 500 mg on Weeks 2, 6, and 10 via |
| | intravenous (IV) administration. Subjects who discontinue from the study |
| | will have a follow-up period of 12 weeks after the last dose. |
| | Response at Week 12 will be defined as reduction from Baseline in CDAI |
| | of ≥100 points. Non-responders at Week 12 should discontinue from the study. |
| | Subjects who complete the 12-week IP and have responded will have the |
| | option to enter OLE. Subjects will continue in OLE until they progress, |
| | withdraw from the study, study termination, or Week 50. During the OLE, |
| | starting at Week 14 visit: |
| | Subjects who achieved deep remission (defined as clinical remission |
| | with CDAI <150, endoscopic remission with SES-CD ≤4 with none of |
| | the segments with score of more than 1, and RHI <3) will continuein the |
| | study and have therapy withdrawal |
| | Upon disease relapse (defined as CDAI ≥220 and either hsCRP ≥5 |
| | mg/L and/or fecal calprotectin ≥250 μg/g), subjects can receive |
| | another course of induction therapy (1000 mg IV followedby 500 mg IV 2, |
| | 6, and 10 weeks after the first infusion) followed by maintenance therapy |
| | of 250 mg IV every 4 weeks (Q4W) for atotal of 50 weeks |
| | Other subjects will be re-randomized to either 250 mg IV Q4W or |
| | 100 mg IV Q4W until Week 50 |
| | The study may be amended by the Sponsor to extend the OLE period |
| | beyond 50 weeks based on emerging safety data. |
| SAMPLE SIZE: | The study is planned to enroll approximately 50 subjects. The sample size |
| | will enable a statistical power of 80%, at 1-sided significance level of 0.025, |
| | to test against the null hypothesis of endoscopic improvement rate of 12%, |
| | assuming the endoscopic improvement rate for A219 is 27%. |
| SUBJECT TYPE: | Male or female subjects ≥18 years of age with moderately to severely |
| | active CD. |
| FORMULATIONS: | A219 will be supplied in 10 mL vials each containing 500 mg A219(60 |
| | mg/mL solution) for IV administration after reconstitution. |
| DOSAGE: | Induction Period: all subjects will receive A219 1000 mg on Week |
| | 0/Day 1, followed by 500 mg IV on Weeks 2, 6, and 10. |
| | Non-responders at Week 12 should be discontinued from the study. |
| | Responders at the end of Week 12 have the option to enter the OLE, where |
| | subjects with deep remission will undergo therapy withdrawal and subjects |
| | without deep remission will be randomized to receive one of the following |
| | regimens until disease progression, withdraw from the study, study |
| | termination, or Week 50: |
| | A219 250 mg IV on Week 14 then Q4W |
| | A219 100 mg IV on Week 14 then Q4W |
| | Subjects who do not respond by the end of Week 12 should be discontinued |
| | from the study. |
| | All subjects who develop disease relapse (defined as CDAI ≥220 and either |
| | hsCRP ≥5 mg/L and/or fecal calprotectin ≥250 μg/g), after therapy |
| | withdrawal will receive another course of induction therapy (1000 mg IV |
| | followed by 500 mg IV 2, 6, and 10 weeks after the first infusion) followed |
| | by maintenance therapy of 250 mg IV Q4W for a total of 50 weeks. |
| ROUTE OF | The study drug will be reconstituted in 250 mL of 0.9% normal saline (NS) |
| ADMINISTRATION: | and will be administered IV over 30 minutes. |
| STUDY ENDPOINTS: | Primary endpoints: |
| | Safety and tolerability: the proportion of subjects reporting adverse |
| | events (AEs), serious adverse events (SAEs), AEs leading to |
| | discontinuation, and markedly abnormal laboratory values |
| | The proportion of subjects with endoscopic improvement, as defined by |
| | decrease in SES-CD ≥50% from Baseline at Week 12 |
| | Secondary endpoints: |
| | The proportion of subjects in clinical remission (CDAI <150) at |
| | Week 12 |
| | The proportion of subjects with endoscopic and clinical improvement, |
| | as defined by decrease in SES-CD ≥50% AND reduction in CDAI ≥100 |
| | points from Baseline at Week 12 |
| | The proportion of subjects with both biomarker and clinical |
| | improvement (decrease in hsCRP OR fecal calprotectin ≥50% from |
| | Baseline, among subjects with at least one elevated biomarker at |
| | Baseline, AND reduction in CDAI ≥100 points from Baseline) at Week 12 |
| | The proportion of subjects with normalization of hsCRP (as defined by |
| | hsCRP < ULN), among subjects with elevated concentrations at Baseline, |
| | at Week 12 |
| | The proportion of subjects with normalization of fecal calprotectin |
| | (as defined by fecal calprotectin < ULN), among subjects with elevated |
| | concentrations at Baseline, at Week 12 |
| | The proportion of subjects in clinical response, as defined by reduction |
| | in CDAI ≥100 points from Baseline at Week 12 |
| | The proportion of subjects with PRO-2 remission (defined as average |
| | daily abdominal pain score ≤1 point and average daily stool |
| | frequency ≤3 points with abdominal pain and stool frequency no worse |
| | than Baseline) at Week 12 |
| | Change in SES-CD score at Week 12 from Baseline |
| | Descriptive summaries of PK and immunogenicity of A219 |
| | Proportion of subjects developing anti-drug antibody (ADA) and |
| | neutralizing antibody (Nab) |
| | Exploratory endpoints: |
| | Change in CDAI and components from Baseline over time |
| | Change in PD markers including TL1A concentrations, EHI, fecal |
| | calprotectin, and hsCRP over time |
| | Change in SES-CD from Baseline at Week 50 |
| | Change in PDAI from Baseline over time |
| | Proportion of subjects with improvement or remission of |
| | enterocutaneous and/or perianal fistula at Week 12 and Week 50 |
| | Change in GHAS and RHI from Baseline to Week 12 and Week 50 |
| | The proportion of subjects with GHAS histologic score ≤4 at Week 12 |
| | and Week 50 |
| | The proportion of subjects with Robarts histologic score <5 at Week |
| | 12 and Week 50 |
| | The proportion of subjects with GHAS histologic remission, defined as |
| | no neutrophils in the epithelium or subscore of 0, at Week 12 and Week 50 |
| | The proportion of subjects with Robarts histologic remission (<3) at |
| | Week 12 and Week 50 |
| | Time to relapse among subjects with deep remission at Week 12 |
| | Proportion of subjects who relapse, among subjects who achieved deep |
| | remission at Week 12 |
| | Change in PRO-2 over time |
| | The proportion of subjects with extraintestinal manifestation through |
| | Week 50 |
| INCLUSION | Subjects are required to meet the following criteria in order to be |
| CRITERIA: | included in the study: |
| | 1. Male or female ≥18 years of age. |
| | 2. Subjects must have had a documented diagnosis of CD (endoscopy + |
| | histology) to be eligible for study participation. For subjects with no |
| | documented confirmation of CD diagnosis or if previous diagnosis isnot |
| | deemed conclusive, CD diagnosis must be confirmed at time of screening |
| | colonoscopy. |
| | 3. Moderately to severely active CD as defined by CDAI of ≥220 and ≤450. |
| | 4. SES-CD score (percentral reading) ≥6 if ileocolonic or colonic |
| | disease; or ≥4 if isolated ileal disease only. |
| | 5. Subjects must satisfy at least one of the following criteria: |
| | a) In the past, had an inadequate response to one or more of the |
| | following treatments: |
| | Oral prednisone ≥40 mg/day (or equivalent) or budesonide ≥9 |
| | mg/day or equivalent or beclomethasone ≥5 mg/day for atleast 2 weeks |
| | Corticosteroid dependence as defined by failed to successfully |
| | taper to <10 mg/day of prednisone or equivalent (i.e., had a flare of |
| | disease) within 3 months of starting therapy, or if relapse occurs within 3 |
| | months of stopping corticosteroids |
| | Immunosuppressants (azathioprine ≥2 mg/kg/day or |
| | 6-mercaptopurine ≥1.0 mg/kg/day, [or documentation of atherapeutic |
| | concentration of 6-thioguanine nucleotide] or methotrexate ≥15 |
| | mg/week) for at least 8 weeks |
| | An approved anti-TNF agent at an approved labeled dose for at |
| | least 8 weeks |
| | An approved anti-integrin (e.g., vedolizumab) at an approved |
| | labeled dose for at least 8 weeks |
| | An approved anti-IL-12/23 (e.g., ustekinumab) at an approved |
| | labeled dose for at least 8 weeks |
| | OR |
| | b) Had been intolerant to one or more_of the above mentioned |
| | treatments (e.g., unable to achieve doses or treatment durations because of |
| | dose-limiting side effects [e.g., leukopenia, psychosis, uncontrolled |
| | diabetes, elevated liver enzymes]) |
| | OR |
| | c) Currently receiving one or more of the following treatments: |
| | Oral Prednisone ≥20 mg/day (or equivalent) or budesonide ≥3 |
| | mg/day for at least 4 weeks |
| | Immunosuppressants [azathioprine ≥2 mg/kg/day or |
| | 6-mercaptopurine ≥1.0 mg/kg/day, (or documentation of a therapeutic |
| | concentration of 6-thioguanine nucleotide)] for atleast 8 weeks |
| | Notes on subjects who have had prior approved biologic therapy(ies)(e.g., |
| | anti-TNF, anti-integrin, and/or anti-IL-12/23): |
| | The study will include a maximum of 70% subjects who have had |
| | prior approved biologic therapy(ies) experience. Upon reaching the |
| | maximum number of allowed biologic experienced subjects (70%), subjects |
| | who have had prior biologic experience will no longer be allowed to enter |
| | the study. |
| | Subjects cannot have had failed (no response, insufficient response, |
| | loss of response, and/or intolerance) >4 approved biologic therapies, |
| | whether of same or different mechanism of action |
| | Subjects previously on clinical trials only (i.e., did not receive |
| | commercial available therapy post-approval) are not considered tohave |
| | received the approved therapy for purpose of this inclusion criteria |
| | 6. For subjects who are women of childbearing potential (WOCBP) |
| | involved in any sexual intercourse that could lead to pregnancy, the |
| | subject has used two highly effective methods of contraception for at least |
| | 4 weeks prior to Day 1 and agrees to continue to use two highly effective |
| | methods of contraception until at least 12 weeks after the last dose of study |
| | drug. |
| | 7. Male subjects must use, with their female partner of childbearing |
| | potential, two highly effective methods of contraception and refrain from |
| | sperm donation from screening to 12 weeks after the last dose of study |
| | drug. |
| | 8. Subjects must meet drug stabilization requirements, as applicable: |
| | a) Oral corticosteroid treatment must be equivalent of ≤20 mg |
| | prednisone or ≤9 mg budesonide or beclomethasone ≤5 mg daily at a |
| | stable dose for at least 2 weeks prior to Day 1 |
| | b) Oral aminosalicylates should be at a stable dose for at least 2 |
| | weeks prior to Day 1 |
| | c) Azathioprine, 6-mercaptopurine, and methotrexate should be at a |
| | stable dose for at least 4 weeks prior to Day 1 |
| | 9. Able to provide written informed consent and understand and comply |
| | with the requirements of the study. |
| EXCLUSION | Subjects with the following characteristics will be excluded from the study: |
| CRITERIA: | Sex and Reproductive Status |
| | 1. WOCBP and men with female partners of childbearing potential who |
| | are unwilling or unable to use two highly effective methods of |
| | contraception to avoid pregnancy for the entire study period and for upto |
| | 12 weeks after the last dose of study drug. |
| | 2. Women who are pregnant or breastfeeding. |
| | 3. Women with a positive pregnancy test on enrollment or prior to Day 1. |
| | Target Disease Exceptions |
| | 4. Diagnosis of ulcerative colitis or indeterminate colitis. |
| | 5. CD isolated to the stomach, duodenum, jejunum, or perianal region, |
| | without colonic and/or ileal involvement. |
| | 6. Suspected or diagnosed intra-abdominal or perianal abscess at |
| | Screening. |
| | 7. Known symptomatic stricture or stenosis not passable in endoscopy |
| | (including pediatric colonoscope). |
| | 8. Current stoma or need for colostomy or ileostomy. |
| | 9. Previous small bowel resection with combined resected length of >100 |
| | cm or previous colonic resection of >2 segments. |
| | 10. Currently receiving total parenteral nutrition. |
| | 11. Surgical bowel resection within 3 months before screening. |
| | 12. Concomitant primary sclerosing cholangitis (PSC). |
| | Medical History and Concurrent Diseases |
| | 13. Past or current evidence of definite low-grade or high-grade colonic |
| | dysplasia that has not been completely removed. |
| | 14. Subjects who are scheduled or anticipate the need for surgery, aside |
| | from dermatologic procedures. |
| | 15. Subjects who have a history of clinically significant drug or alcohol |
| | abuse. |
| | 16. Concomitant illness that in the opinion of the Investigator, is likely |
| | to require systemic glucocorticosteroid therapy during the study (e.g., |
| | moderate to severe asthma). |
| | 17. Current symptoms of severe, progressive, or uncontrolled renal, |
| | hepatic, hematological, pulmonary, cardiac, neurological, |
| | ophthalmologic, or cerebral disease. Concomitant medical conditionsthat |
| | in the opinion of the Investigator might place the subject at unacceptable |
| | risk for participation in this study. |
| | 18. Subjects with a history of cancer within the last 5 years (other than |
| | non-melanoma skin cell cancers cured by local resection). Existing non- |
| | melanoma skin cell cancers must be removed prior to enrollment. Subjects |
| | with carcinoma in situ or localized cervical cancer, treated with definitive |
| | surgical intervention, are allowed. |
| | 19. Subjects at risk for tuberculosis (TB). Specifically, subjects with: |
| | a) A history of active TB |
| | b) Current clinical, radiographic, or laboratory evidence of active TB |
| | c) Latent TB which was not successfully treated. Subjects with a |
| | positive TB screening test indicative of latent TB will not be eligible for the |
| | study unless active TB infection has been ruled out, and an appropriate |
| | course of intervention for latent TB has been initiated at least 2 weeks prior |
| | to Day 1, and no evidence of active TB on chest x-ray during screening. |
| | 20. Subjects with any serious bacterial infection within the last 3 months, |
| | unless treated and resolved with antibiotics, or any chronic bacterial |
| | infection (such as chronic pyelonephritis, osteomyelitis, and |
| | bronchiectasis). |
| | 21. Female subjects who have had a breast cancer screening that is |
| | suspicious for malignancy, and in whom the possibility of malignancy |
| | cannot be reasonably excluded following additional clinical, laboratory, or |
| | other diagnostic evaluations. |
| | 22. Subjects with any active infections (excluding fungal infections of nail |
| | beds) including, but not limited to, those that require IV antimicrobial |
| | treatment 4 weeks or oral antimicrobial treatment 2 weeks prior to |
| | randomization. Subjects with evidence of Human Immunodeficiency |
| | Virus (HIV), Hepatitis B, or Hepatitis C infection detected during screening |
| | are also excluded, but subjects with successfully treated Hepatitis C with no |
| | recurrence for ≥1 year are allowed. Subjects with active documented or |
| | suspected COVID-19 infection within 4 weeks of randomization or |
| | asymptomatic SARS-CoV-2 PCR test within 2 weeksof randomization are |
| | excluded. |
| | 23. Subjects with herpes zoster reactivation or cytomegalovirus (CMV) |
| | that resolved less than 2 months prior to signing informed consent. |
| | 24. Subjects who have received any live vaccines within 3 months of the |
| | anticipated first dose of study medication or who will have need of alive |
| | vaccine at any time during the study. |
| | Physical and Laboratory Test Findings |
| | 25. Positive stool Polymerase Chain Reaction (PCR) or culture for enteric |
| | pathogens. |
| | 26. Stool positive for Clostridium difficile (C. difficile) toxin. Subjects who |
| | are positive can be retested after the completion of a full course of treatment |
| | for C. difficile infection. |
| | 27. Any of the following lab values: |
| | a) Hemoglobin (Hgb) <8.0 g/dL (80 g/L) |
| | b) White blood cell (WBC) <2,500/mm³ (2.5 × 10⁹/L) |
| | c) Neutrophils <1,000/mm³ (1 × 10⁹/L) |
| | d) Platelets <100,000/mm³ (100 × 10⁹/L) |
| | d) Serum creatinine >2 times ULN |
| | e) Serum alanine aminotransferase (ALT) or aspartate |
| | aminotransferase (AST) >2 times ULN |
| | f) Any other laboratory test results that, in the opinion of the |
| | Investigator, might place the subject at unacceptable risk for |
| | participation in this study. |
| | Prohibited Therapies and/or Medications |
| | 28. Failed (no response, insufficient response, loss of response, and/or |
| | intolerance) >4 approved biologic therapies (anti-TNF, anti-integrin, anti- |
| | IL12/23), whether of same or different mechanism of action. |
| | 29. Any marketed biologic within 8 weeks for anti-TNF agents and 12 |
| | weeks for anti-integrin agents (e.g., vedolizumab) and ustekinumabprior |
| | to Day 1 or if drug level per therapeutic dose monitoring is greater than |
| | lower limit of detection. |
| | 30. Any biologic immunomodulators used for CD or other conditions |
| | within 8 weeks or 5 half-lives, whichever is longer, prior to Day 1 or if drug |
| | level per therapeutic dose monitoring is greater than lower limit ofdetection. |
| | 31. Rituximab within 1 year prior to Day 1. |
| | 32. Parenteral corticosteroids within 4 weeks or rectal administration of |
| | corticosteroids within 2 weeks prior to Day 1. |
| | 33. Rectal administration of 5-ASA within 2 weeks prior to Day 1. |
| | 34. Tacrolimus, cyclosporine, mycophenolate mofetil (CellCept ®), |
| | immunoadsorption columns (such as Prosorba columns), D Penicillamine, |
| | Leflunomide, Thalidomide, chronic use of non-steroidal anti-inflammatory |
| | agents (NSAIDs), and aspirin >81 mg/day within 2 weeks prior to Day 1. |
| | 35. Other investigational chemical agent within 30 days or other |
| | investigational biologic agent within 8 weeks or 5 half-lives (whicheveris |
| | longer) of entry into the IP. |
| | 36. Prior exposure to A219. |
| | Other Exclusion Criteria |
| | 37. Prisoners or subjects who are compulsorily detained (involuntarily |
| | incarcerated) for treatment of either a psychiatric or physical |
| | (e.g., infectious disease) illness. |
| | 38. Legal or mental incapacitation, or inability to understand and comply |
| | with the requirements of the study. |
| Statistical Methods: | Statistical methods will be detailed in the Statistical Analysis Plan (SAP). |
| | The SAP will provide details about methods of analysis and the specific |
| | planned analyses, and will be prepared and approved by Prometheus |
| | Biosciences and its designees before study database lock. |
| | The analysis populations are defined as follows: |
| | Full analysis set (FAS): all subjects treated with Baseline SES-CD score |
| | Safety analysis set: all subjects treated |
| | The following analyses will be performed: |
| | Efficacy: |
| | The primary efficacy endpoint, endoscopic improvement at Week 12, will |
| | be used to assess the efficacy of A219. The proportion of subjects in FAS |
| | with endoscopic improvement will be tested against the null hypothesis of |
| | endoscopic improvement rate of 12%, at a 1-sided significance level of |
| | 0.025. If significant, the 1^{st} secondary endpoint of proportion of subjects |
| | achieving clinical remission will be tested against thenull hypothesis of |
| | clinical remission rate of 16%, at a 1-sided significance level of 0.025. |
| | The point estimates for the primary and secondary endpoints will be |
| | calculated along with 90% confidence interval for FAS and by companion |
| | diagnostic (CDx) status (CDx+ or CDx−). |
| | Adverse Events: |
| | AEs will be coded using the most current version of Medical Dictionary for |
| | Regulatory Activities (MedDRA ®). |
| | A by-subject AE data listing, including verbatim term, preferred term (PT), |
| | system organ class (SOC), treatment, severity, seriousness criteria, |
| | relationship to drug, and action taken, will be provided. |
| | The number of subjects experiencing treatment-emergent adverse events |
| | (TEAEs) and number of TEAEs will be summarized by treatment using |
| | frequency counts in safety analysis set. |
| | Medical History, chest x-ray, electrocardiogram (ECG), and physical |
| | examination will be listed by subject. |
| | Changes in ECGs and physical examinations will be described in the text of |
| | the final report. |
| | Concomitant Medications: |
| | Concomitant medications will be coded using the most current World |
| | Health Organization (WHO) drug dictionary and listed by treatment. |
| | Pharmacokinetics: |
| | Summary statistics of A219 concentrations and ADA by visit. |

### Example 24: Formulation Verification Study

Exemplary formulations provided herein were placed on long-term stability studies (up to six months). This Example summarizes the results of these storage stability studies.

Materials used in this study include A219 at various concentrations as indicated.

### Methods and Procedures

UV Analysis. The sample absorbance and sample concentration were measured by standard UV absorbance instrument, using an extinction coefficient of 1.41 mL·mg−1 cm−1, and correcting background scattering.

pH Analysis. Before the start of analysis, the pH probe was calibrated with three pH standards ordered from fisher. The pH of the formulation will be measured by inserting the pH probe into the sample and waiting until the measured value has stabilized, which can take up to 1 to 2 minutes.

Osmotic Analysis. The osmotic analysis was performed using an Advanced Instruments Osmo 1. At the start of analysis, a reference standard at 290 mOsm is analyzed to ensure the instrument is working properly. After the reference standard has passed the samples are then analyzed. 20 μL of sample material is removed and analyzed by the Osmo 1.

Viscosity. The viscosities of the samples were measured using m-Vroc from Rheosense (San Ramon, CA, USA). The dynamitic viscosity of sample was calculated by flowing the samples past three difference pressure sensors. The linear regression of the pressure drop from the three sensors was then used to calculate dynamitic viscosity of the samples. The instrument was calibrated and the dynamitic viscosity of the samples were measured according to manufacturer's instructions and industry standards. The analysis parameters for sample concentrations ranging from 60 to ˜230 mg/mL are listed in Table 30:

TABLE 30
Viscosity parameters for assessing protein samples

| A219 | | | |
|---|---|---|---|
| concentration | Shear | Measurement | Waiting |
| (mg/mL) | Rate, 1/s | Time, s | Time, s |
| 60 | 1000 | 1.9 | 3 |
| 150 | 1000 | 1.9 | 3 |
| 175 | 1000 | 1.9 | 3 |
| 200 | 1000 | 1.9 | 3 |

Size Exclusion Chromatography (SEC). The SEC method was used to measure the stability of the protein samples.

Cation Exchange Chromatography (CEX). The CEX was also used to measure the stability of the protein samples.

Flow Imaging (FlowCam). Measurements of particle counts in the samples were made using a Model VS-1 FlowCAM flow imaging system (SN 551) with Sony SX90 camera and C70 pump with a 1 mL syringe (Fluid Imaging Technologies). The system qualification consisted of obtaining acceptable bead counts using an NIST certified count standard (Pharm Trol, Thermo, catalogue no. CS3800-15 or similar) along with acceptable procedural blanks (water). The acceptance criteria used for the count standard was 3800±15% and no more than 1 count/mL greater than or equal to 10 μm for the water blank. Samples were visually assessed during the sample run and if needed adjustments were made to optimize the results of each run. An x-y flow plot was recorded for each sample run.

Study Design. The verification study examined formulations with A219 concentrations ranging from 60 to 200 mg/mL as shown in Table 31 (formulations 1-9 as Form. 1-9 in the table, or F01-F08 in this Example). The storage stability study plan is shown in Table 32.

TABLE 31
Formulations tested in this study

| | A219 | | | | | | |
|---|---|---|---|---|---|---|---|
| | Concen- | | | | | | |
| | tration | | Acetate | Sucrose | Lys-HCL | NaCl | PS 20 |
| Form. | (mg/mL) | pH | (mM) | (mM) | (mM) | (mM) | (%) |
| 1 | 60 | pH 6.5, 20 mM PO4, 5% Sucrose, 20 mM | | | | | 0.01 |
| | | Glycine | | | | | |
| 2 | 60 | 5.3 | 20 | 240 | 25 | 0 | 0.02 |
| 3 | 150 | 5.3 | 20 | 240 | 25 | 0 | 0.02 |
| 4 | 175 | 5.3 | 20 | 240 | 25 | 0 | 0.02 |
| 5 | 200 | 5.3 | 20 | 240 | 25 | 0 | 0.02 |
| 6 | 150 | 5.3 | 20 | 220 | 0 | 40 | 0.02 |
| 7 | 175 | 5.3 | 20 | 220 | 0 | 40 | 0.02 |
| 8 | 200 | 5.3 | 20 | 220 | 0 | 40 | 0.02 |

TABLE 32
Study Design

| Temp. | | 1 Month | 2 Month | 3 Month | 6 Month |
|---|---|---|---|---|---|
| ° C. | T0 | (1 M) | (2 M) | (3 M) | (6 M) |
| 5 | X | X | X | X | X |
| 25 | X | X | X | X | |

Notes for Table 32:
Initial Time point (T0): pH, osmolality, viscosity; at the end of each storage condition: visual inspection SEC and CEX and others as described in this Example.
Each time point (1 M, 2 M, 3 M, 6 M): Visual Inspection, SEC and CEX and others as described in this Example

### Results

The control (T0) samples listed in Table 31 were characterized by visual inspection, osmolality (osmo), pH, protein concentration, viscosity, SEC, CEX and Flow Cam. The remaining times were analyzed by SEC and CEX except that the last time point of each temperature in Table 32 was characterized by the same measurements in TO.

### Visual Characterization

The bulk material used for these studies had a slight yellow tint, but was otherwise clear with no visual particles observed. The formulations at TO were visually inspected and were clear with no visible particles observed. At 60 mg/mL, formulations 1 and 2, had slight yellow tint, the tint became more intense as the concentration increased from 60 mg/mL to 200 mg/mL. A summary of the visual observations can be found in Table 33. Throughout the study, no visible particles were observed and the samples remained clear under all conditions.

TABLE 33
Visual characterization of the stability samples

| | Visual | Visual | | Visual | Visual | | Visual |
|---|---|---|---|---|---|---|---|
| Form. | (T0) | (1 M 5) | Visual(1 M 25) | (2 M 25) | (3 M 5) | Visual(3 M 25) | (6 M 5) |
| 1 | C, NP, NC | C, NP, NC | C, NP, NC | C, NP, NC | C, NP, NC | C, NP, NC | C, NP, NC |
| 2 | C, NP, NC | C, NP, NC | C, NP, NC | C, NP, NC | C, NP, NC | C, NP, NC | C, NP, NC |
| 3 | C, NP, NC | C, NP, NC | C, NP, NC | C, NP, NC | C, NP, NC | C, NP, NC | C, NP, NC |
| 4 | C, NP, NC | C, NP, NC | C, NP, NC | C, NP, NC | C, NP, NC | C, NP, NC | C, NP, NC |
| 5 | C, NP, NC | C, NP, NC | C, NP, NC | C, NP, NC | C, NP, NC | C, NP, NC | C, NP, NC |
| 6 | C, NP, NC | C, NP, NC | C, NP, NC | C, NP, NC | C, NP, NC | C, NP, NC | C, NP, NC |
| 7 | C, NP, NC | C, NP, NC | C, NP, NC | C, NP, NC | C, NP, NC | C, NP, NC | C, NP, NC |
| 8 | C, NP, NC | C, NP, NC | C, NP, NC | C, NP, NC | C, NP, NC | C, NP, NC | C, NP, NC |

C = Clear,
NP = No Particles,
NC = No Color

### Osmolality

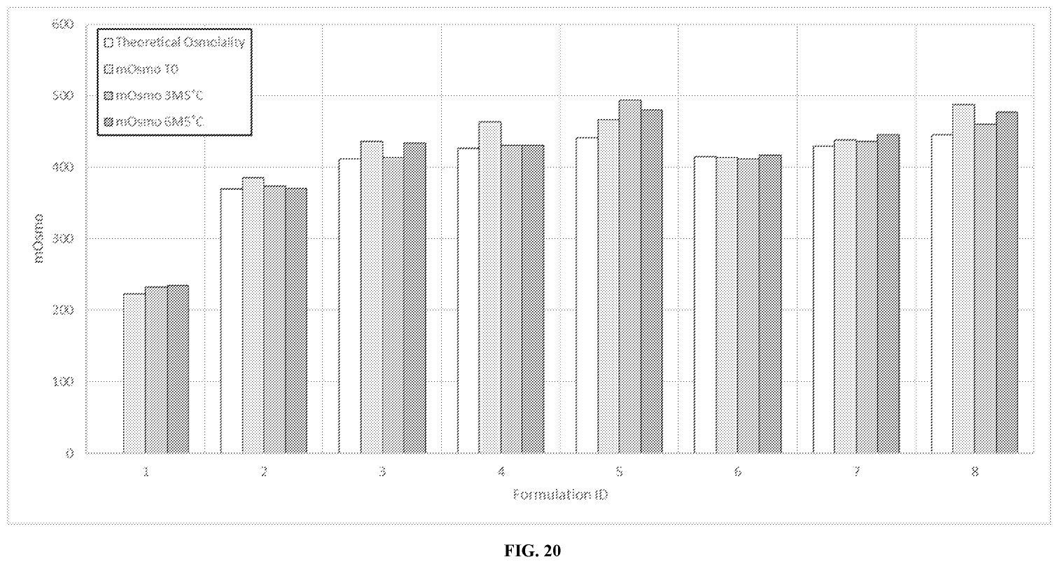

The osmotic pressure was measured for the stability samples at TO, 3 and 6 months (Table 34). In addition, the theoretical osmolality was calculated for all formulations, except for formulation 1. The osmotic pressure for the samples ranged from 223 to 487 mOsmol/kgH₂O for the highest protein concentrations (Table 34). The differences in the osmotic pressure between theoretical and measured become larger as the protein concentration increased from 60 to 200 mg/ml, reflecting the increasing contribution from the protein. Over time, the osmolality values do increases slightly for some formulations (FIG. 20), but the differences are relatively minor.

TABLE 34
Osmotic pressure was measured at T0, 3 and 6 months

| | Theoretical | | mOsmo3M5° | mOsmo3M25° | mOsmo6M5° |
|---|---|---|---|---|---|
| Form. | Osmolality | mOsmoT0 | C. | C. | C. |
| 1 | n.a | 223 | 232 | 227 | 235 |
| 2 | 369 | 385 | 374 | 371 | 370 |
| 3 | 412 | 436 | 414 | 425 | 434 |
| 4 | 426 | 463 | 431 | 450 | 431 |
| 5 | 441 | 466 | 494 | 472 | 480 |
| 6 | 415 | 414 | 412 | 416 | 417 |
| 7 | 429 | 438 | 436 | 429 | 445 |
| 8 | 445 | 487 | 460 | 472 | 477 |

### Protein Concentration

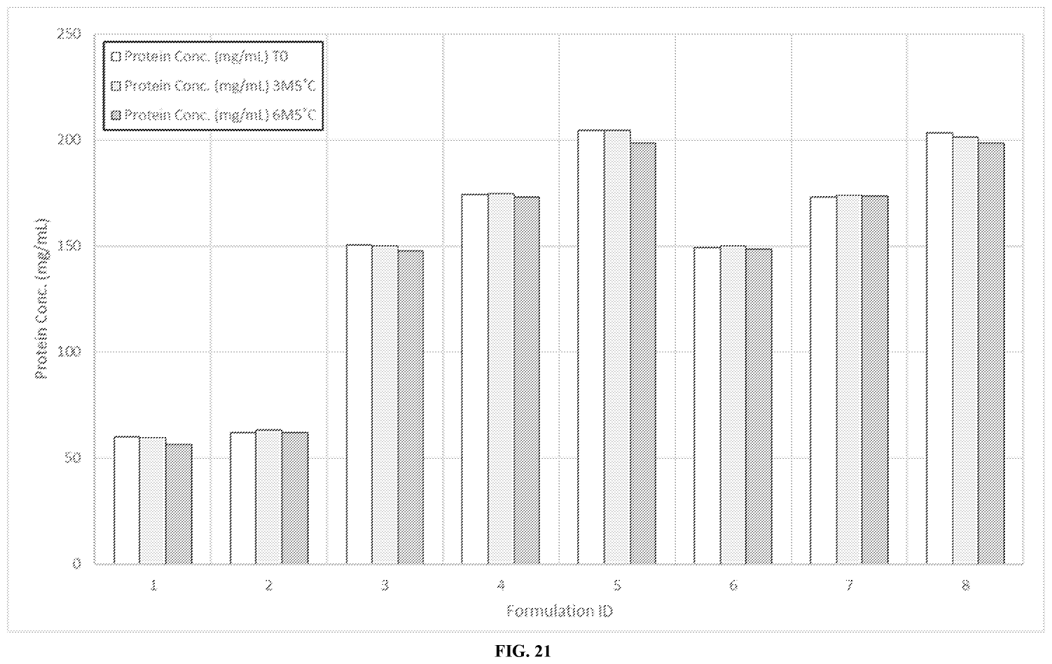

The A219 protein concentration was measured to evaluate the stability of samples at T0, 3 and 6 months as shown in Table 35. Most of the values appear to be unchanged within the estimated error of the protein concentration method, indicating that the A219 is stable in these formulations (Table 35 and FIG. 21). The plot of the measured A219 protein concentrations in each sample after 0, 3 and 6 months shows that the concentrations are fairly constant and likely do not reflect any substantive changes in protein content (FIG. 21). Moreover, the protein concentrations were all within 5% of the target concentration for the formulation.

TABLE 35
Protein concentration was measured at T0, 3 and 6 months

| | | T0 | 3 M5° C. | 3 M25° C. | 6 M5° C. |
|---|---|---|---|---|---|
| Form. | (mg/mL) | (mg/mL) | (mg/mL) | (mg/mL) | (mg/mL) |
| 1 | 60 | 60.05 | 59.73 | 59.53 | 56.54 |
| 2 | 60 | 61.88 | 63.01 | 62.13 | 61.87 |
| 3 | 150 | 150.32 | 150.13 | 149.89 | 147.56 |
| 4 | 175 | 174.18 | 174.91 | 175.03 | 173.18 |
| 5 | 200 | 204.54 | 204.34 | 204.26 | 198.39 |
| 6 | 150 | 149.45 | 150.24 | 149.22 | 148.45 |
| 7 | 175 | 173.16 | 173.95 | 173.69 | 173.51 |
| 8 | 200 | 203.17 | 201.42 | 203.39 | 198.66 |

### pH Measurements

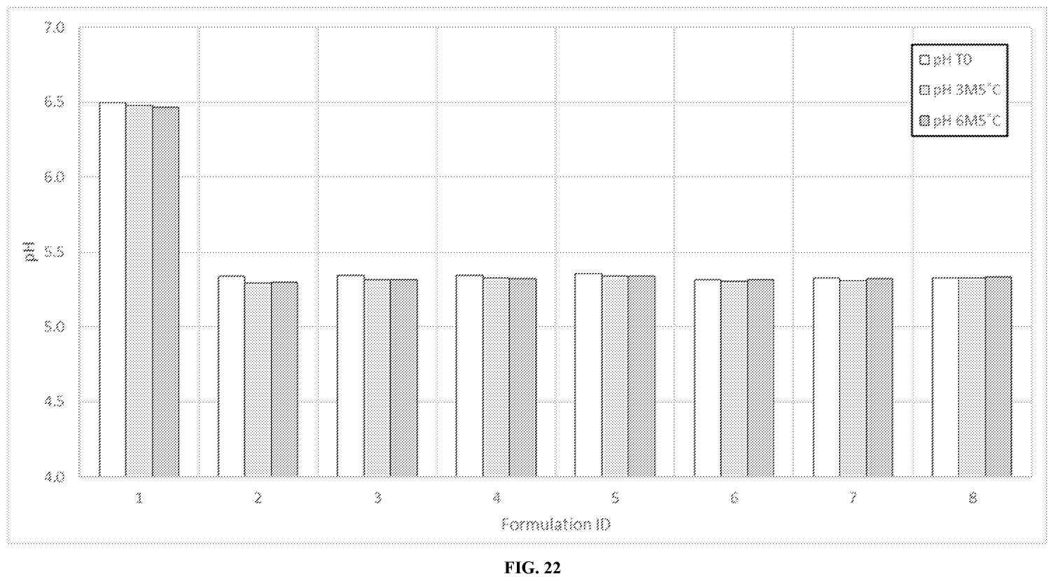

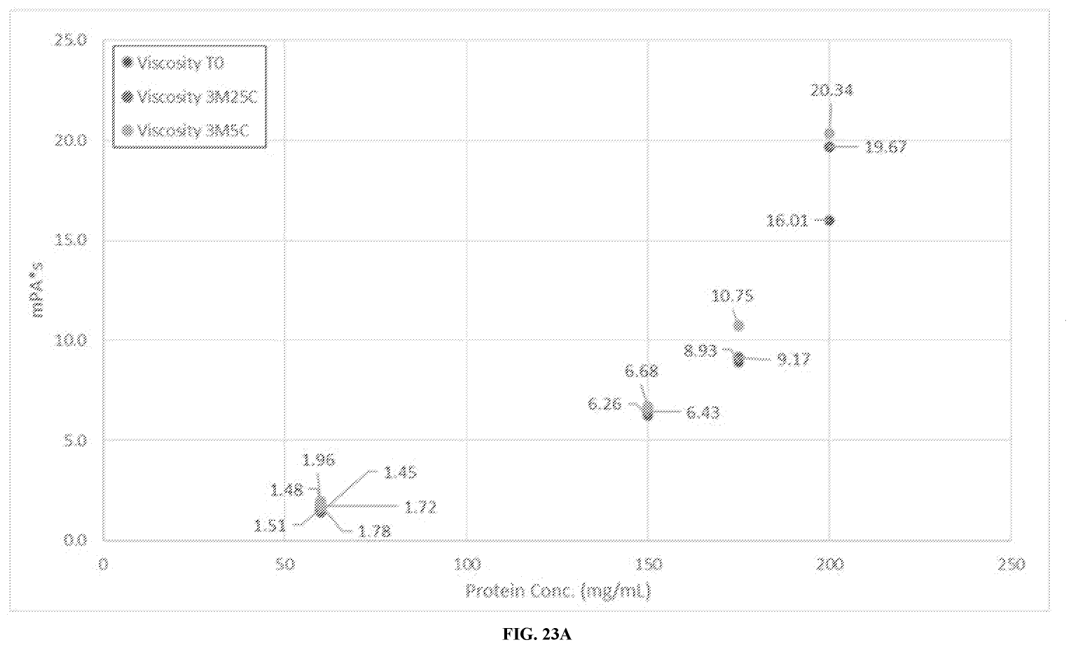

The pH values were measured to evaluate the stability of samples at the 0, 3 and 6 month time points (Table 36). The measured pH values were all within less than 0.1 of the target pH for the formulation. The constancy of the pH values is shown in FIG. 22.

TABLE 36
pH values measured at T0, 3 and 6 months.

| Form. | pH | pH T0 | pH 3 M5° C. | pH 6 M5° C. | pH 3 M25° C. |
|---|---|---|---|---|---|
| 1 | 6.5 | 6.50 | 6.48 | 6.47 | 6.46 |
| 2 | 5.3 | 5.34 | 5.29 | 5.30 | 5.31 |
| 3 | 5.3 | 5.35 | 5.32 | 5.32 | 5.33 |
| 4 | 5.3 | 5.35 | 5.33 | 5.33 | 5.34 |
| 5 | 5.3 | 5.36 | 5.34 | 5.34 | 5.35 |
| 6 | 5.3 | 5.32 | 5.31 | 5.32 | 5.32 |
| 7 | 5.3 | 5.33 | 5.31 | 5.32 | 5.33 |
| 8 | 5.3 | 5.33 | 5.33 | 5.33 | 5.34 |

### Viscosity Measurements

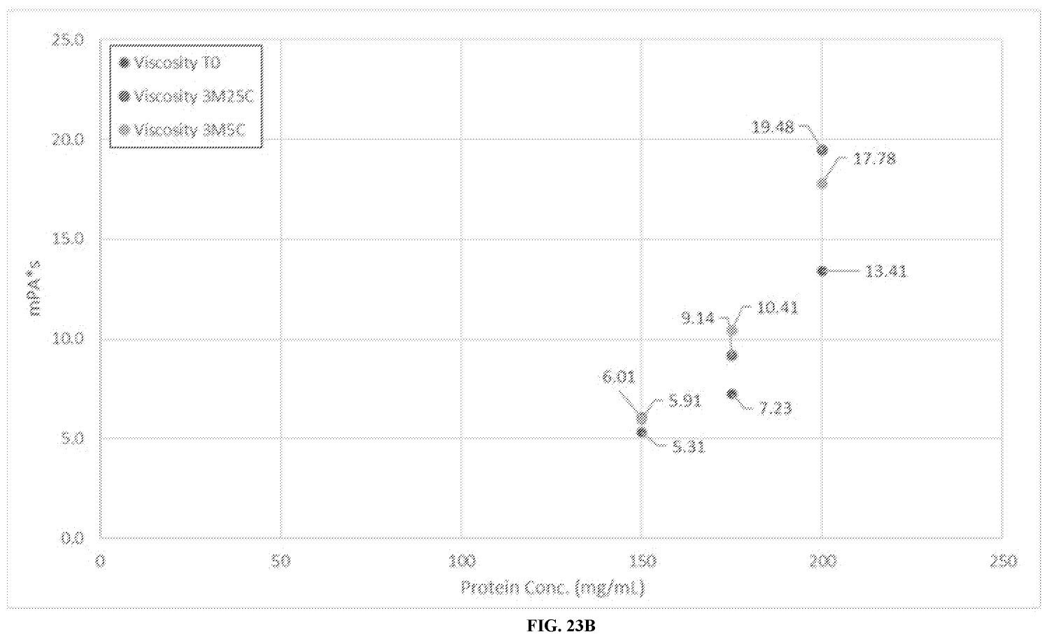

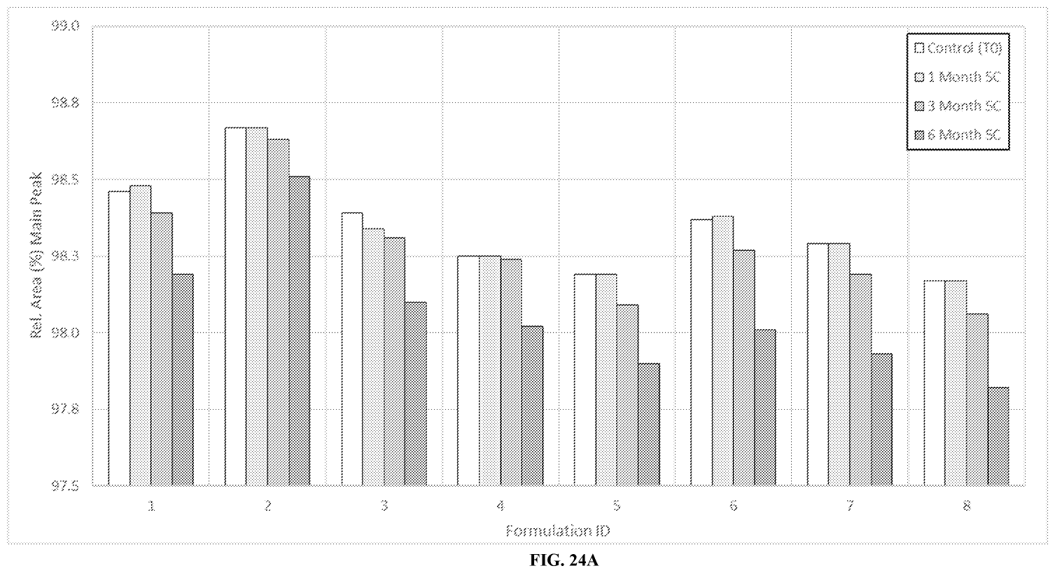

The viscosities were measured to evaluate the stability of A219 samples of various formulations at TO, and after 3 and 6 months as shown in Table 37. FIGS. 23A and 23B show the graphical representation of the viscosity data vs protein concentration at TO, consistent with an exponential response and with the viscosity of mAbs behaving as function of concentration.

For formulations 6-8, the viscosity data ranges from about 5.3 to 13.4 mPa*s as protein increased from ˜150 to ˜200 mg/mL. By comparison, formulations 3-5 have a somewhat higher viscosity, ranging from about 6.3 to 16.0 mPa*S over the same protein concentration range. Upon storage, some of the formulations do exhibit slightly higher viscosity values, possibly due to slightly increased levels of aggregates (see below).

TABLE 37
Viscosity was measured at T0, 3 and 6 months.

| | | | | | Lys- | | | | | |
|---|---|---|---|---|---|---|---|---|---|---|
| | | | Acetate | Sucrose | HCL | | PS 20 | Viscosity | Viscosity | Viscosity |
| Form. | (mg/mL) | pH | (mM) | (mM) | (mM) | NaCl (mM) | (%) | T0 | 3 M 25 C. | 6 M 5 C. |
| 1 | 60 | 20 mM Po4, 5% Sucrose, 20 mM Glycine, | | | | | 0.01 | 1.51 | 1.45 | 1.78 |
| | | 0.01% PS 20 | | | | | | | | |
| 2 | 60 | 5.3 | 20 | 240 | 25 | 0 | 0.02 | 1.48 | 1.72 | 1.96 |
| 3 | 150 | 5.3 | 20 | 240 | 25 | 0 | 0.02 | 6.26 | 6.43 | 6.68 |
| 4 | 175 | 5.3 | 20 | 240 | 25 | 0 | 0.02 | 8.93 | 9.17 | 10.75 |
| 5 | 200 | 5.3 | 20 | 240 | 25 | 0 | 0.02 | 16.01 | 19.67 | 20.34 |
| 6 | 150 | 5.3 | 20 | 220 | 0 | 40 | 0.02 | 5.31 | 6.01 | 5.91 |
| 7 | 175 | 5.3 | 20 | 220 | 0 | 40 | 0.02 | 7.23 | 9.14 | 10.41 |
| 8 | 200 | 5.3 | 20 | 220 | 0 | 40 | 0.02 | 13.41 | 19.48 | 17.78 |

Stability measurements by Size Exclusion Chromatography (SEC)

The stability of the A219 samples was characterized by size exclusion chromatography (SEC). At T0, the monomer content was >98% for these samples (Table 38). After two months at 25° C., the monomer content only slightly decreases, with all formulations retaining >97% monomer. Even after three months at 25° C., the monomer contents remain near 97%. When stored at 5° C., the loss of monomer (primarily due to formation of higher molecular weight (HMW) species) averages only about 0.2-0.4% (Table 39).

TABLE 38
T0, 1 and 2 month SEC results for A219 samples

| | T0 (Rel. Area %) | | | 1 M 5° C. (Rel. Area %) | | | 1 M 25° C. (Rel. Area %) | | | 2 M 25° C. (Rel. Area %) | | |
|---|---|---|---|---|---|---|---|---|---|---|---|---|
| | | Main | | | Main | | | Main | | | Main | |
| Form. | HMW | Peak | LMW | HMW | Peak | LMW | HMW | Peak | LMW | HMW | Peak | LMW |
| 1 | 1.46 | 98.46 | 0.08 | 1.44 | 98.48 | 0.08 | 1.85 | 98.02 | 0.13 | 2.04 | 97.77 | 0.19 |
| 2 | 1.25 | 98.67 | 0.08 | 1.26 | 98.67 | 0.07 | 1.4 | 98.44 | 0.15 | 1.52 | 98.25 | 0.23 |
| 3 | 1.54 | 98.39 | 0.07 | 1.59 | 98.34 | 0.08 | 1.91 | 97.95 | 0.14 | 2.12 | 97.66 | 0.21 |
| 4 | 1.67 | 98.25 | 0.07 | 1.67 | 98.25 | 0.07 | 2.02 | 97.84 | 0.14 | 2.26 | 97.53 | 0.21 |
| 5 | 1.74 | 98.19 | 0.06 | 1.75 | 98.19 | 0.07 | 2.20 | 97.66 | 0.14 | 2.50 | 97.29 | 0.21 |
| 6 | 1.57 | 98.37 | 0.06 | 1.56 | 98.38 | 0.06 | 2.01 | 97.85 | 0.14 | 2.28 | 97.50 | 0.22 |
| 7 | 1.65 | 98.29 | 0.06 | 1.65 | 98.29 | 0.06 | 2.14 | 97.73 | 0.14 | 2.44 | 97.35 | 0.21 |
| 8 | 1.76 | 98.17 | 0.06 | 1.77 | 98.17 | 0.06 | 2.34 | 97.52 | 0.14 | 2.64 | 97.15 | 0.21 |

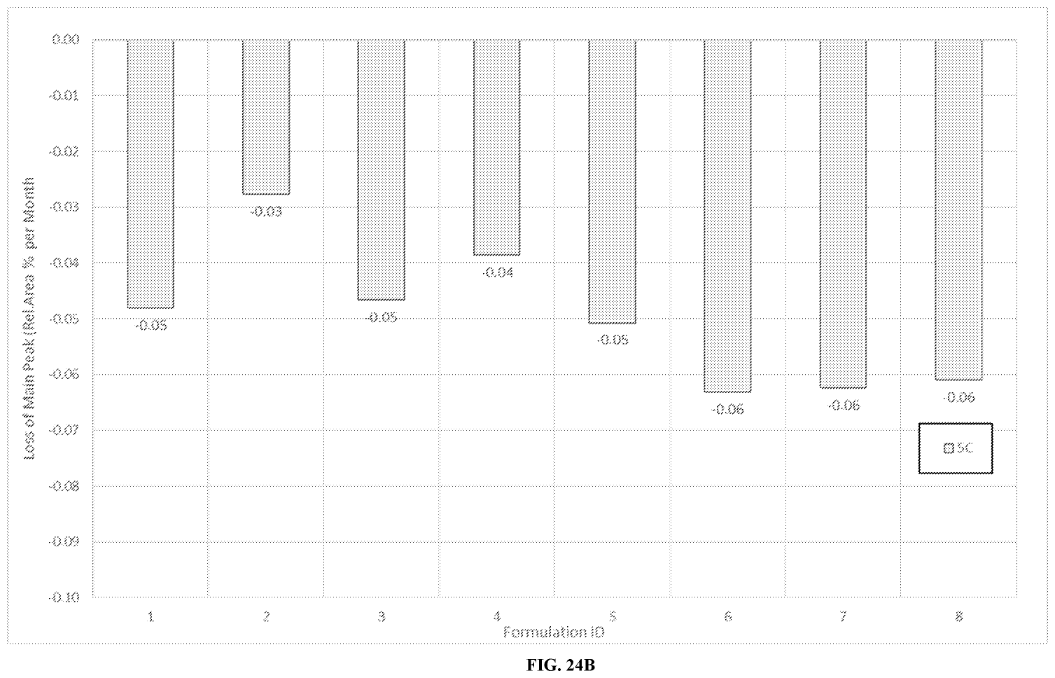

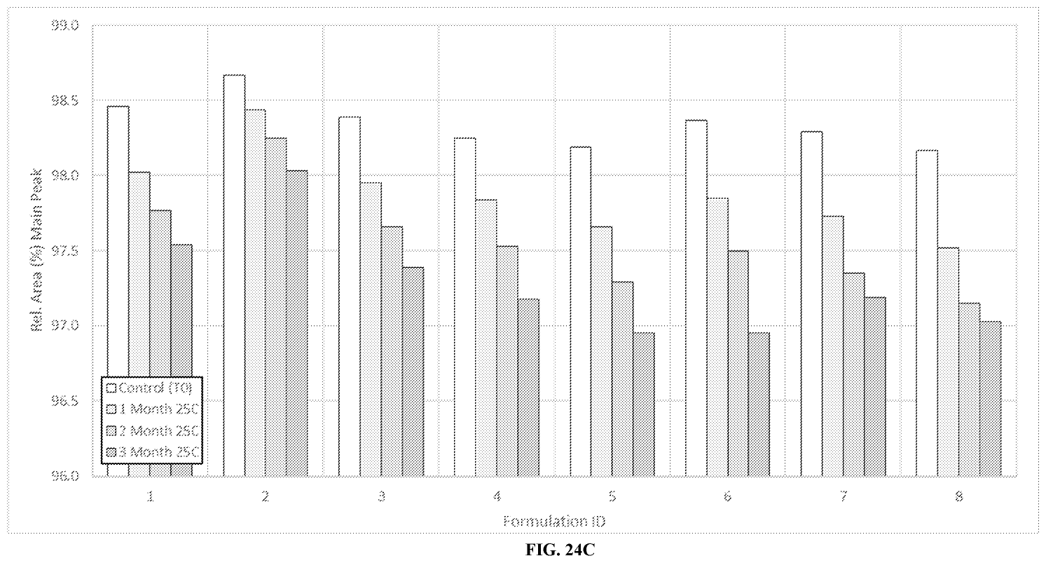

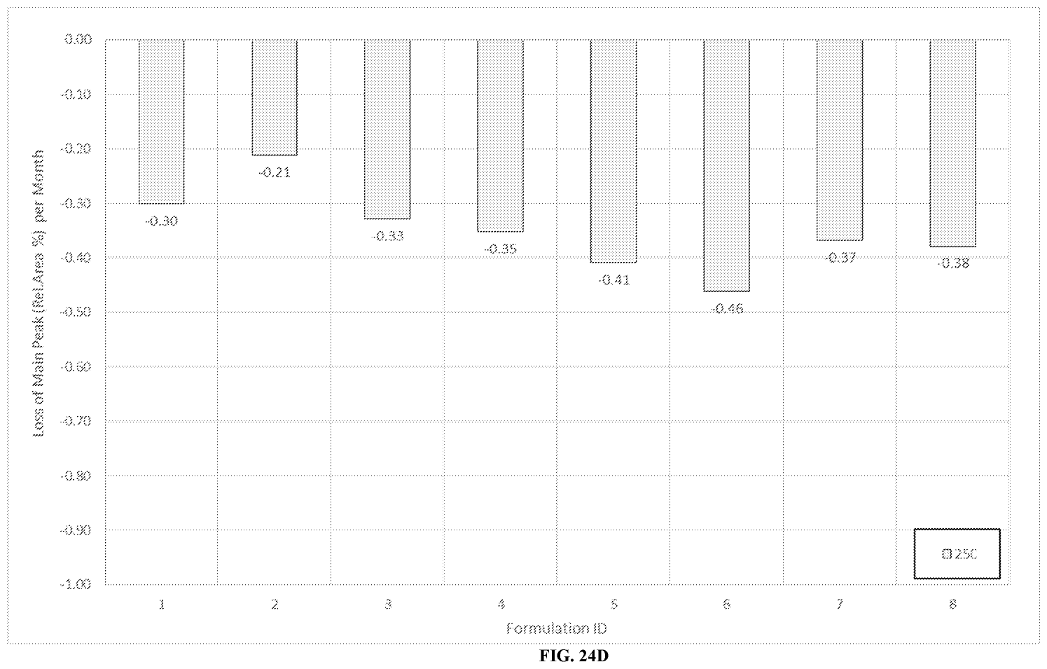

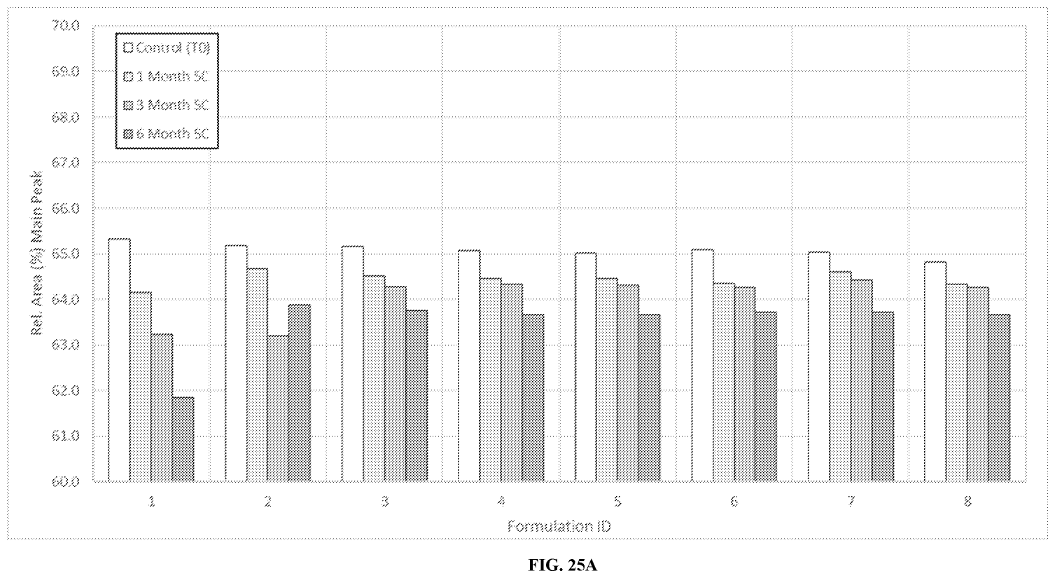

The small loss of monomer is shown in the graph in FIG. 24A. It appears that formation of aggregates (HMW species) is increased somewhat at higher concentrations for high concentration formulations 3-8. The overall monomer loss per month for samples stored at 5° C. is only about 0.04 to 0.06% FIG. 24B. The monomer levels for the 25° C. samples are provided in FIG. 24C. The average loss per month for these samples is about 0.3 to 0.4% per month as shown in FIG. 24D. Based on these data, there would be less than 1% loss of monomer at 5° C. over two years and less than 5% loss of monomer when stored at 25° C.

TABLE 39
Summary of 3 and 6 month SEC results for A219 samples

| | 3 M 5° C. (Rel. Area %) | | | 3 M 25° C. (Rel. Area %) | | | 6 M 5° C. (Rel. Area %) | | |
|---|---|---|---|---|---|---|---|---|---|
| Form. | HMW | Main Peak | LMW | HMW | Main Peak | LMW | HMW | Main Peak | LMW |
| 1 | 1.54 | 98.39 | 0.07 | 2.20 | 97.54 | 0.27 | 1.72 | 98.19 | 0.09 |
| 2 | 1.29 | 98.63 | 0.07 | 1.65 | 98.03 | 0.31 | 1.40 | 98.51 | 0.09 |
| 3 | 1.62 | 98.31 | 0.07 | 2.32 | 97.39 | 0.29 | 1.81 | 98.10 | 0.09 |
| 4 | 1.69 | 98.24 | 0.07 | 2.53 | 97.18 | 0.29 | 1.89 | 98.02 | 0.09 |
| 5 | 1.84 | 98.09 | 0.07 | 2.76 | 96.95 | 0.29 | 2.01 | 97.90 | 0.09 |
| 6 | 1.67 | 98.27 | 0.06 | 2.76 | 96.95 | 0.29 | 1.89 | 98.01 | 0.09 |
| 7 | 1.74 | 98.19 | 0.07 | 2.52 | 97.19 | 0.29 | 1.98 | 97.93 | 0.09 |
| 8 | 1.88 | 98.06 | 0.07 | 2.68 | 97.03 | 0.29 | 2.09 | 97.82 | 0.09 |

### Stability Measured by Cation Exchange Chromatography (CEX)

The stability of the A219 samples were characterized by cation exchange chromatography (CEX). The CEX data for the 0, 1, and 2 month time points are summarized in Table 40. The relative area of the main peak started near 65%. Over time, this decreased, primarily due to increases in the acidic species, indicating some hydrolytic change, such as deamidation, was occurring. Formulation 1 (F01) shows the greatest changes.

TABLE 40
A219 samples characterized by cation exchange chromatography at T0, 1 and 2 month

| | T0 (Rel. Area %) | | | 1 M 5° C. (Rel. Area %) | | | 1 M 25° C. (Rel. Area %) | | | 2 M 25° C. (Rel. Area %) | | |
|---|---|---|---|---|---|---|---|---|---|---|---|---|
| | | Main | | | Main | | | Main | | | Main | |
| Form. | Acid | Peak | Basic | Acid | Peak | Basic | Acid | Peak | Basic | Acid | Peak | Basic |
| 1 | 32.21 | 65.33 | 2.46 | 33.47 | 64.16 | 2.38 | 40.44 | 56.81 | 2.75 | 46.26 | 51.08 | 2.66 |
| 2 | 32.15 | 65.18 | 2.67 | 32.67 | 64.67 | 2.66 | 36.63 | 60.01 | 3.35 | 40.42 | 56.13 | 3.45 |
| 3 | 32.23 | 65.17 | 2.60 | 32.68 | 64.52 | 2.80 | 36.43 | 59.89 | 3.67 | 40.20 | 56.07 | 3.73 |
| 4 | 32.14 | 65.08 | 2.78 | 32.69 | 64.46 | 2.84 | 36.37 | 59.87 | 3.76 | 40.01 | 56.15 | 3.84 |
| 5 | 32.24 | 65.01 | 2.75 | 32.62 | 64.46 | 2.92 | 36.38 | 59.74 | 3.88 | 39.97 | 56.10 | 3.93 |
| 6 | 32.28 | 65.09 | 2.63 | 32.72 | 64.36 | 2.92 | 36.42 | 59.77 | 3.80 | 40.00 | 56.15 | 3.85 |
| 7 | 32.26 | 65.04 | 2.69 | 32.58 | 64.60 | 2.83 | 36.23 | 59.95 | 3.82 | 39.95 | 56.22 | 3.83 |
| 8 | 32.44 | 64.82 | 2.74 | 32.74 | 64.33 | 2.94 | 36.26 | 59.80 | 3.94 | 39.78 | 56.21 | 4.02 |

TABLE 41
A219 samples characterized by cation exchange
chromatography at T0 3 and 6 month

| | 3 M 5° C. (Rel. Area %) | | | 3 M 25° C. (Rel. Area %) | | | 6 M 5° C. (Rel. Area %) | | |
|---|---|---|---|---|---|---|---|---|---|
| | | Main | | | Main | | | Main | |
| Form. | Acid | Peak | Basic | Acid | Peak | Basic | Acid | Peak | Basic |
| 1 | 34.39 | 63.23 | 2.38 | 51.34 | 45.88 | 2.78 | 35.56 | 61.84 | 2.61 |
| 2 | 34.18 | 63.2 | 2.62 | 44.3 | 51.79 | 3.9 | 33.33 | 63.89 | 2.78 |
| 3 | 32.84 | 64.28 | 2.88 | 43.84 | 51.89 | 4.27 | 33.23 | 63.76 | 3.01 |
| 4 | 32.77 | 64.34 | 2.89 | 43.87 | 51.66 | 4.47 | 33.2 | 63.67 | 3.13 |
| 5 | 32.72 | 64.32 | 2.96 | 43.68 | 51.67 | 4.66 | 33.17 | 63.66 | 3.17 |
| 6 | 32.83 | 64.26 | 2.91 | 43.69 | 51.83 | 4.48 | 33.22 | 63.73 | 3.05 |
| 7 | 32.74 | 64.42 | 2.84 | 6.97 | 50.98 | 0.98 | 33.17 | 63.73 | 3.1 |
| 8 | 32.72 | 64.26 | 3.02 | 43.49 | 51.81 | 4.7 | 33.18 | 63.66 | 3.16 |

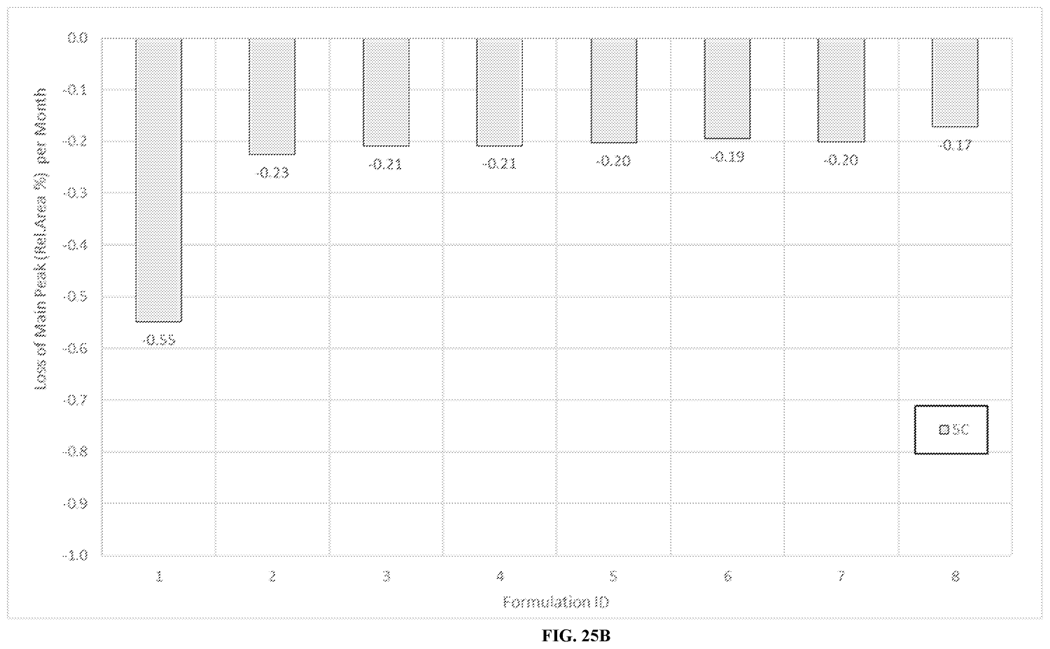

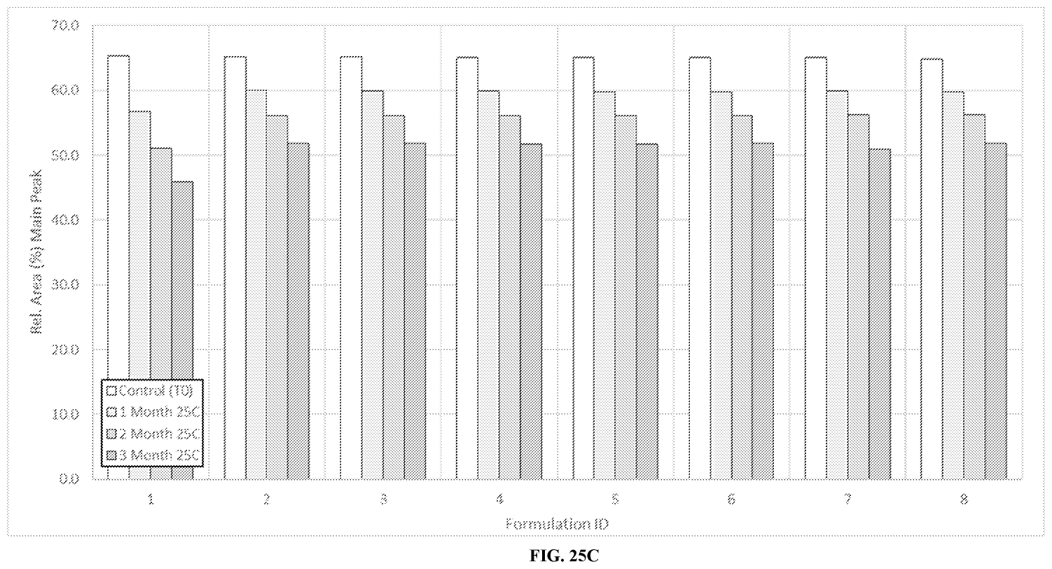

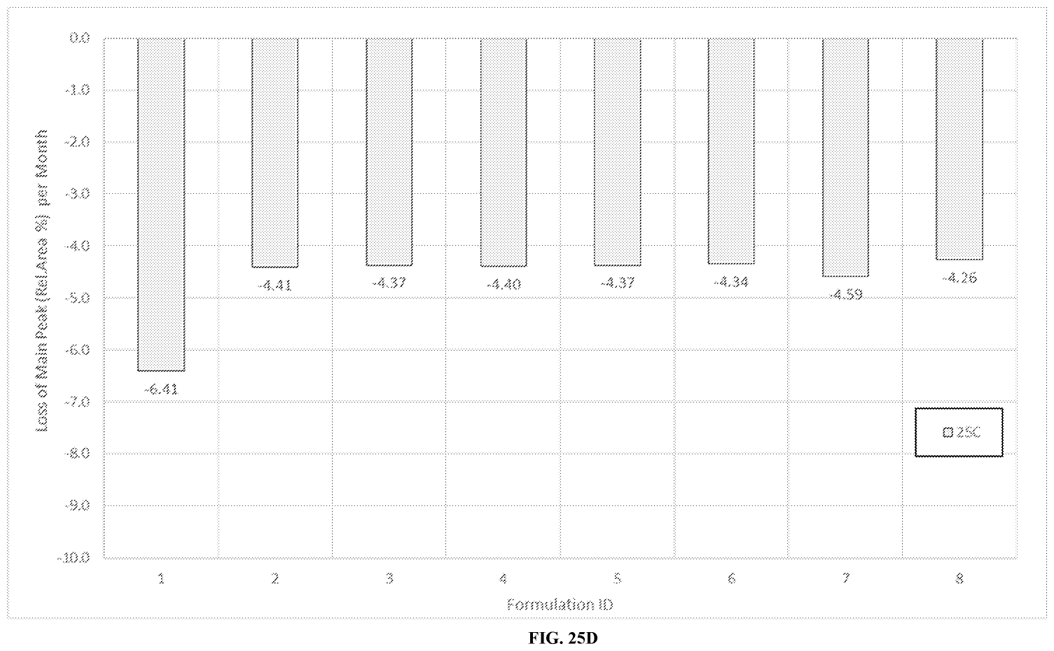

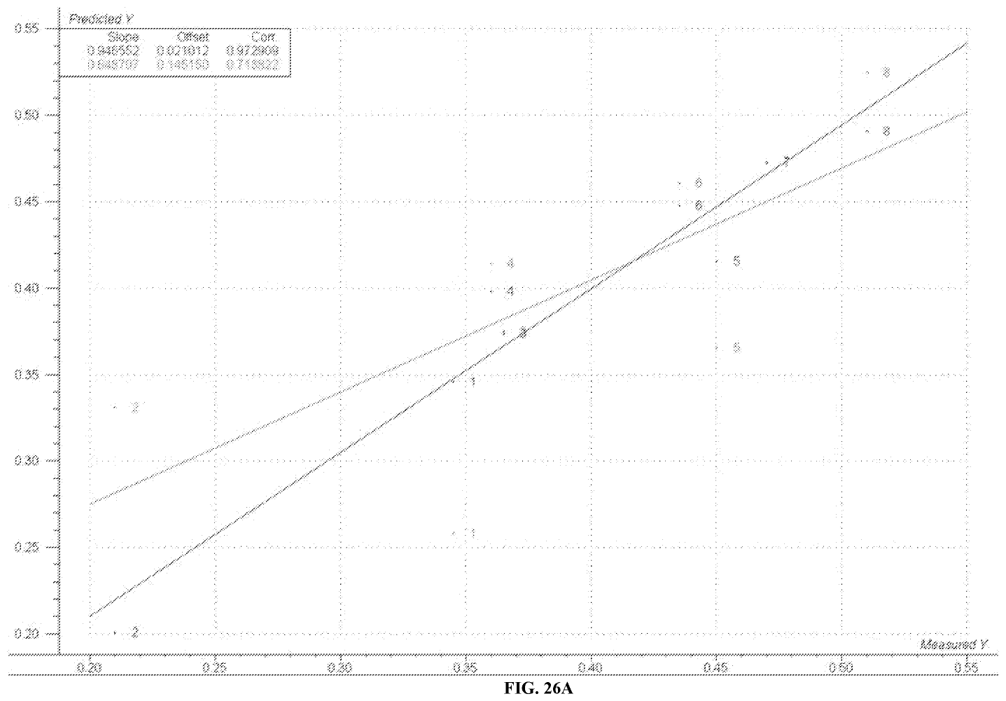

After three months at 25° C., the main peak averaged near 51%, while F01 continues to show greater decrease with only about 46% main peak relative area (Table 41). By comparison, samples stored at 5° C. exhibit very little decrease in CEX measurements. Even after six months, the relative area of the CEX main peak remains near 63% for formulations 2-8. Changes in the relative area of the CEX main peak are shown in FIG. 25A. Meanwhile, all of the high concentration (A219 at or above 150 mg/ml) formulations showed comparable stability by CEX. The rate of loss at 5 C is provided in FIG. 25B and formulations 2-8 have very good stability as determined by CEX.

The CEX stability profiles at 25° C. are seen in FIG. 25C. The decrease in the main peak, presumably by hydrolytic changes, are more pronounced at 25° C. than at 5° C. The rates of decrease per month at 25° C. are about 20 times greater than at 5° C. (FIG. 25D).

### Stability Measurements by FlowCAM Flow Imaging Analysis

The stabilities of these samples were characterized by FlowCAM, which counts the number of subvisible particles (SVPs) in various size bins. The levels of SVPs are all reported in particle per mL. At T0, the particle counts are higher for F01 than the other formulations (Table 42). However, at one month/5 C, the particle levels for F01 were not comparable to the other preparations. Results for the FlowCAM analysis for samples held at 25° C. after one and two months are shown in Table 43. Levels in all of the formulations remain relatively low after two months at 25° C. (Table 43) and three months as well (Table 44). The SVP levels for samples held at 5° C. for three months appear to be even slightly lower than for the corresponding 25° C. samples (Table 44). Finally, samples held for six months at 5° C. were analyzed, and the levels of SVPs remained low as shown (Table 45).

TABLE 42
FlowCAM Analysis for T0 and 1 Month stability samples

| | Time Point: 0 | | | | | Time Point: 1 Month 5° C. | | | | |
|---|---|---|---|---|---|---|---|---|---|---|
| | GTE 2 | GTE | GTE | GTE | GTE | GTE | GTE | GTE | GTE | GTE |
| Form | um | 3 um | 5 um | 10 um | 25 um | 2 um | 3 um | 5 um | 10 um | 25 um |
| F1 | 141512 | 63887 | 25534 | 3612 | 439 | 3771 | 2096 | 1450 | 474 | 13 |
| F2 | 7163 | 2994 | 1226 | 250 | 79 | 3244 | 1331 | 447 | 131 | 26 |
| F3 | 2901 | 1647 | 698 | 184 | 26 | 1332 | 594 | 343 | 92 | 13 |
| F4 | 2413 | 1226 | 553 | 184 | 26 | 2466 | 1253 | 633 | 132 | 39 |
| F5 | 3640 | 1872 | 1081 | 527 | 92 | 949 | 343 | 277 | 145 | 0 |
| F6 | 2848 | 1371 | 738 | 250 | 39 | 4813 | 2690 | 1358 | 448 | 105 |
| F7 | 13676 | 8800 | 4876 | 1105 | 0 | 18122 | 8000 | 3103 | 463 | 40 |
| F8 | 4432 | 1979 | 1188 | 317 | 53 | 1543 | 949 | 395 | 79 | 0 |

TABLE 43
FlowCAM Analysis for 1 and 2 Month stability samples

| | Time Point: 1 Month 25° C. | | | | | Time Point: 2 Month 25° C. | | | | |
|---|---|---|---|---|---|---|---|---|---|---|
| | GTE | GTE | GTE | GTE | GTE | GTE | GTE | GTE | GTE | GTE |
| Form | 2 um | 3 um | 5 um | 10 um | 25 um | 2 um | 3 um | 5 um | 10 um | 25 um |
| F1 | 4023 | 1900 | 779 | 251 | 13 | 3864 | 2308 | 1464 | 567 | 158 |
| F2 | 23330 | 11129 | 4660 | 897 | 26 | 1094 | 554 | 303 | 66 | 13 |
| F3 | 2453 | 1212 | 513 | 65 | 13 | 1305 | 751 | 355 | 52 | 0 |
| F4 | 8854 | 4024 | 1952 | 435 | 53 | 435 | 317 | 198 | 79 | 13 |
| F5 | 2526 | 1477 | 658 | 215 | 40 | 514 | 304 | 172 | 93 | 0 |
| F6 | 4749 | 2071 | 1003 | 238 | 40 | 382 | 171 | 79 | 26 | 0 |
| F7 | 1477 | 686 | 396 | 145 | 13 | 686 | 197 | 52 | 39 | 13 |
| F8 | 3798 | 2163 | 1174 | 356 | 79 | 356 | 237 | 171 | 105 | 26 |

TABLE 44
FlowCAM Analysis for 3 Month stability samples

| | Time Point: 3 Month 5° C. | | | | | Time Point: 3 Month 25° C. | | | | |
|---|---|---|---|---|---|---|---|---|---|---|
| | GTE | GTE | GTE | GTE | GTE | GTE | GTE | GTE | GTE | GTE |
| Form | 2 um | 3 um | 5 um | 10 um | 25 um | 2 um | 3 um | 5 um | 10 um | 25 um |
| F1 | 1807 | 791 | 237 | 26 | 0 | 4340 | 1846 | 778 | 184 | 65 |
| F2 | 1081 | 448 | 145 | 66 | 13 | 3509 | 1292 | 369 | 66 | 26 |
| F3 | 3877 | 1464 | 528 | 53 | 0 | 1490 | 619 | 237 | 52 | 13 |
| F4 | 1002 | 528 | 251 | 119 | 0 | 1925 | 725 | 198 | 53 | 13 |
| F5 | 1029 | 277 | 92 | 13 | 13 | 857 | 370 | 185 | 40 | 0 |
| F6 | 2796 | 1239 | 487 | 144 | 26 | 2136 | 937 | 172 | 40 | 0 |
| F7 | 1609 | 830 | 395 | 26 | 0 | 5144 | 2282 | 884 | 132 | 0 |
| F8 | 2347 | 962 | 303 | 26 | 0 | 2268 | 1029 | 502 | 40 | 40 |

TABLE 45
FlowCAM Analysis for6 Month stability samples

| | Time Point: 6 Month 5° C. | | | | |
|---|---|---|---|---|---|
| Form | GTE 2 um | GTE 3 um | GTE 5 um | GTE 10 um | GTE 25 um |
| F1 | 6079 | 2967 | 1332 | 462 | 92 |
| F2 | 2110 | 989 | 409 | 145 | 66 |
| F3 | 6345 | 2849 | 1081 | 237 | 79 |
| F4 | 1055 | 565 | 249 | 91 | 39 |
| F5 | 1846 | 777 | 250 | 52 | 13 |
| F6 | 5327 | 2148 | 1067 | 263 | 92 |
| F7 | 2321 | 1134 | 461 | 224 | 13 |
| F8 | 620 | 263 | 39 | 13 | 13 |

### PLS Analysis for the 2 Month Samples

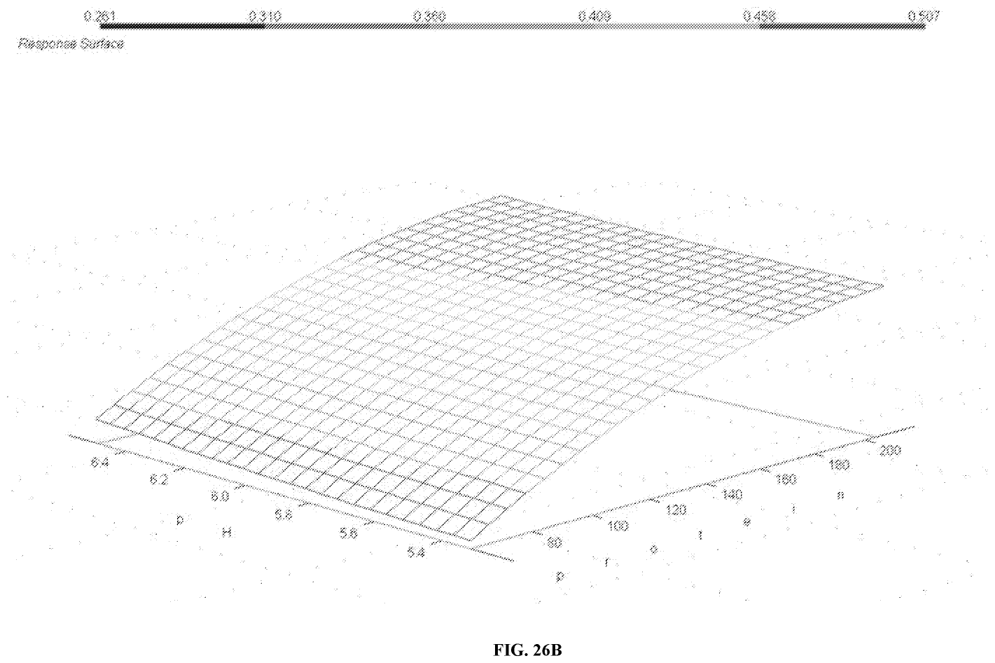

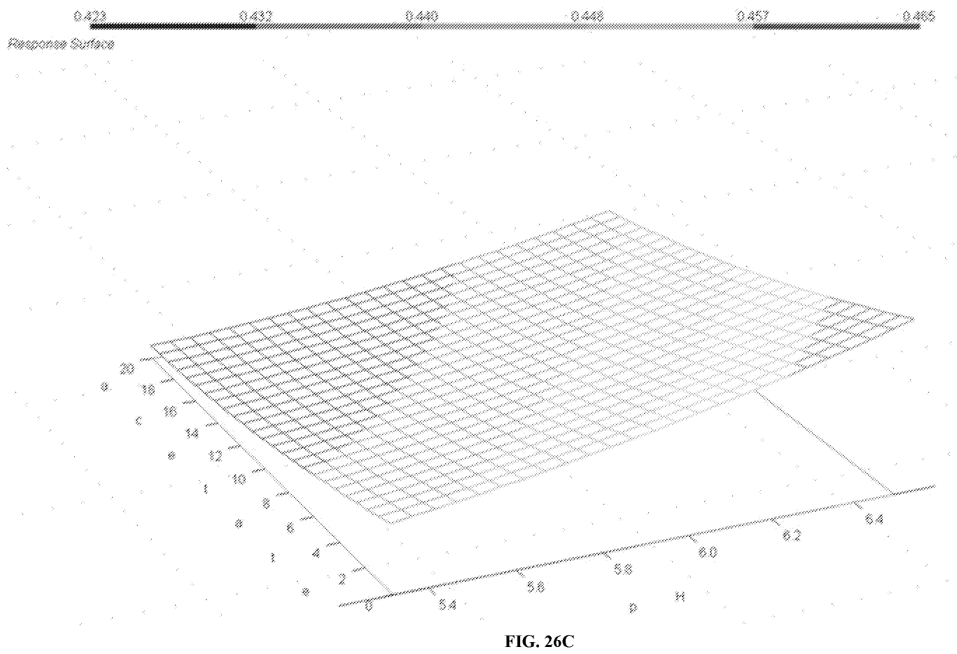

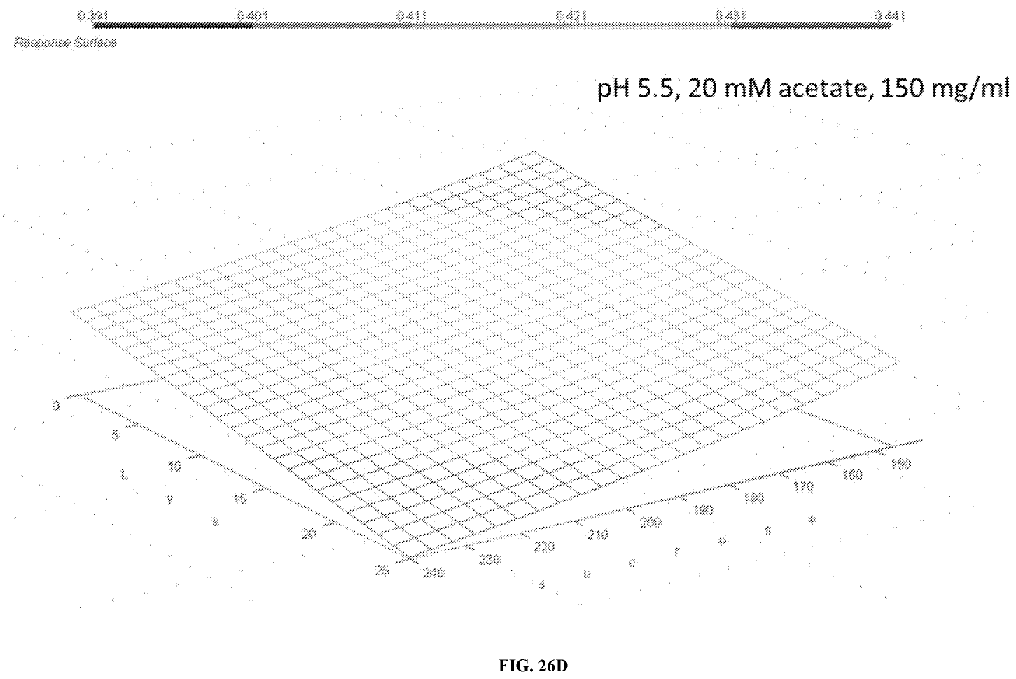

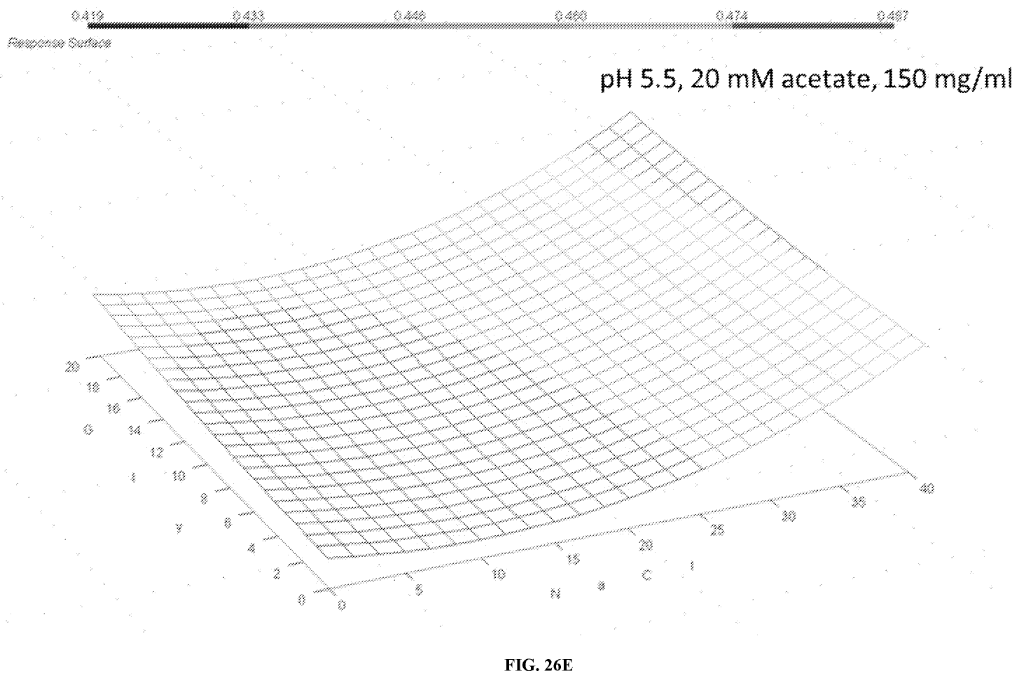

The 25° C. 2 month data was used to constructed the PLS models. The first PLS model used the Loss MP by SEC after two months at 25° C. as the endpoint (FIG. 26A). The correlation coefficient for the calibration set was 0.975 while the r-value for the validation set was 0.776, indicating a model of reasonable quality. The PLS model indicates the significant factors influencing the stability of A219 include protein concentration, sucrose, etc.

This model indicates that monomer loss (e.g., aggregation) is greater at higher protein concentrations (FIG. 26B). This effect is much more pronounced than the pH effect. The model predicts that lower pH and addition of acetate buffer reduces monomer loss upon storage at 25° C. (FIG. 26C). Both sucrose and Lys were found to be effective stabilizers against aggregation (FIG. 26D), while the impact of NaCl and Gly is small (FIG. 26E).

Eight different formulations were placed in longer-term storage at 5° C. and 25° C., and evaluated. Using the acetate buffer system, the pH of each acetate formulation remains essentially unchanged upon storage. As for the high concentration A219 samples, the formulations with sucrose/NaCl clearly reduced viscosity relative to the sucrose/Lys formulations, corresponding to about ˜3 cP at 200 mg/ml. The rate of loss of monomer for samples in the formulations 2-8 stored at 5° C. is quite small, with a total loss of monomer after two years predicted to be <1% for all of the high concentration formulations.

These compositions appear to have little proclivity to form particles. There is no evidence of formation of visible particles and levels of SVPs remains low, even after six months of storage. Overall, these high concentration formulations appear to be quite stable and they appear to support the use of a 200 mg/ml formulation.

### Example 25: Additional Formulation Verification Study

An Exemplary A219 formulation (60 mg/mL A219 in 20 mM sodium phosphate, 5% (w/v) sucrose, 85 mM glycine, 0.01% (w/v) polysorbate 20, pH 6.5) was placed on long-term stability studies. This Example summarizes the results of the storage stability studies for such exemplary formulation.

The long-term stability condition tested for anti-TL1A (e.g. A219) was 5±3° C. (Upright). Accelerated stability condition at 25° C./60% RH (Upright) was also performed. Testing is performed per the stability protocol presented in Table 46 and Table 47 below. The methods used for stability testing and acceptance criteria are presented in Table 48 and Table 49. In addition, a full ICH stability study was also performed for the A219 formulation listed in this Example (ICH referring to International Council on Harmonization of Technical Requirements for Pharmaceuticals for Human Use).

TABLE 46
A219 formulation Storage Conditions and Sampling Times

| | Time Point (Months) | | | | | | | | |
|---|---|---|---|---|---|---|---|---|---|
| Storage Condition | 0 | 1 | 3 | 6 | 9 | 12 | 18 | 24 | 36 |
| 2-8° C. | X | X | X | X | X | X, Y | X | X, Y | X, Y |
| 25° C./60% RH | | X | X | X | | | | | |
| −20° C. | | X | X | X | X | X, Y | X | X, Y | X, Y |

TABLE 47
Additional A219 formulation Storage Conditions and SamplingTimes

| | Time Point (Months) | | | | | | | | | |
|---|---|---|---|---|---|---|---|---|---|---|
| Storage Condition | 0 | 1 | 3 | 6 | 9 | 12 | 18 | 24 | 30 | 36 |
| 2-8° C. | A, B, | A | A | A | A | A, B, | A | A, B, | A | A, B, |
| ambient relative humidity, | C, D, | | | | | C, D, | | C, D, | | C, D, |
| Upright orientation | E | | | | | E | | E | | E |
| 25° C./60% relative | | A | A | A, B, | | | | | | |
| humidity, | | | | D | | | | | | |
| Upright orientation | | | | | | | | | | |

TABLE 48
Methods used for Stability Testing in Table 46

| Testing | | |
|---|---|---|
| Group | Testing Parameter | Method |
| X | Appearance | Visual inspection |
| | pH | Potentiometric |
| | Concentration | Content UV Spectrophotometry |
| | | (280 nm) |
| | Non-reduced and reduced | Capillary electrophoresis |
| | CE SDS | |
| | SEC | SEC-UPLC |
| | icIEF | Capillary electrophoresis |
| | Affinity | Antigen binding ELISA |
| | Biological activity | Cell-based potency assay |
| | (Test initiated at | |
| | 12 month time point) | |
| Y | Subvisible Particulates | USP <787> |

TABLE 49
Methods used for Stability Testing in Table 47

| Testing | | |
|---|---|---|
| Group | Testing Parameter | Method |
| A | Appearance | Visual inspection |
| | pH | Potentiometric |
| | Concentration | Content UV Spectrophotometry |
| | | (280 nm) |
| | Extractable volume | Gravimetric |
| | Non-reduced and reduced | Capillary electrophoresis |
| | CE SDS | |
| | SEC | SEC-UPLC |
| | icIEF | Capillary electrophoresis |
| | Affinity | Antigen binding ELISA |
| | Biological activity | Cell-based potency assay |
| | (Test initiated at | |
| | 6 month time point) | |
| B | Endotoxin | USP <85> |
| C | Sterility | USP <71> |
| D | Subvisible Particulates | USP <787> |
| E | Extractable volume | Gravimetric |

Results from the stability study are shown in Table 50, Table 51, and Table 52. Briefly, no significant change in A219 protein quality was observed for up to 12 months of storage at −20° C. or 2-8° C. and there have been no out of specification findings or changes in the key analysis parameters. The antigen binding affinity and biological activity are well preserved at the 6 month, 25° C. time point. The biophysical changes seen indicate that the formulation is suitable for A219 monoclonal antibody at elevated storage temperatures for prolonged periods.

Results from the ICH stability study are shown in Table 53, and Table 54. Briefly, no significant change in A219 protein quality was observed for up to 6 months of storage at 2-8° C. and there have been no out of specification findings or changes in the key analysis parameters. The antigen binding affinity is well preserved at the 6 month, 25° C. time point. The biophysical changes seen indicate that the formulation is suitable for A219 monoclonal antibody at elevated storage temperatures for prolonged periods.

TABLE 50
Stability Study: Data for Storage Conditions −20° C.

| | | Timepoints (in months) | | | | | | | |
|---|---|---|---|---|---|---|---|---|---|
| Test | Initial | 1 | 3 | 6 | 9 | 12 | 18 | 24 | 36 |

| | | | | | | |
|---|---|---|---|---|---|---|
| Appearance | Conforms | Conforms | Conforms | Conforms | Conforms | Conforms |
| | | No particulates | No particulates | No particulates | No particulates | No particulates |
| | | observed | observed | observed | observed | observed |
| pH | 6.6 | 6.6 | 6.6 | 6.6 | 6.6 | 6.6 |
| Concentration | 63.6 mg/mL | 63.9 mg/mL | 64.6 mg/mL | 61.3 mg/mL | 62.1 mg/mL | 61.8 mg/mL |
| Non-reduced | Intact IgG: 96.9% | Intact IgG: | Intact IgG: | Intact IgG: | Intact IgG: | Intact IgG: |
| and reduced | Total IgG: 96.6% | 96.3% | 96.6% | 95.3% | 95.0% | 94.9% |
| CE-SDS | HC: 64.5% | TotalIgG: | TotalIgG: | TotalIgG: | TotalIgG: | TotalIgG: |
| | LC: 32.2% | 96.3% | 96.1% | 96.6% | 96.2% | 96.1% |
| | | HC: 64.3% | HC: 64.1% | HC: 64.6% | HC: 63.7% | HC: 64.0% |
| | | LC: 32.0% | LC: 31.9% | LC: 32.0% | LC: 32.4% | LC: 32.1% |
| SEC | 97.4% Monomer | 97.1% Monomer | 97.2% Monomer | 96.2% Monomer | 96.1% Monomer | 96.5% |
| | 2.0% Aggregate | 2.2% Aggregate | 2.2% Aggregate | 3.1% Aggregate | 3.2% Aggregate | Monomer |
| | | | | | | 2.9% |
| | | | | | | Aggregate |
| plicIEF | pI = 8.8 | pI = 8.9 | pI = 8.9 | pI = 8.9 | pI = 8.9 | pI = 8.9 |
| | Conforms to Ref | Conforms to Ref | Conforms to Ref | Conforms to Ref | Conforms to Ref | Conforms to |
| | Std. Main peak = | Std. Main peak = | Std. Main peak = | Std. Main peak = | Std. Main peak = | Ref Std. Main |
| | 55.9% | 57.1% | 55.6% | 54.3% | 53.0% | peak = 52.8% |
| | Acidic = 42.4% | Acidic = 40.9% | Acidic = 42.2% | Acidic = 44.1% | Acidic = 44.0% | Acidic = 45.2% |
| | Basic = 1.7% | Basic = 2.0% | Basic = 2.1% | Basic = 1.6% | Basic = 2.0% | Basic = 2.1% |
| Affinity | 111% | 127% | 95% | 142% | 134% | 94% |
| Biological | NP | NP | NP | NP | NP | 95% |
| activity | | | | | | |

NP = not performed

TABLE 51
Stability Study: Data for Storage Conditions 2-8° C.

| | | Timepoints (in months) | | | | | | | |
|---|---|---|---|---|---|---|---|---|---|
| Test | Initial | 1 | 3 | 6 | 9 | 12 | 18 | 24 | 36 |

| | | | | | | |
|---|---|---|---|---|---|---|
| Appearance | Conforms | Conforms | Conforms | Conforms | Conforms | Conforms |
| | | No particulates | No particulates | No particulates | No particulates | No particulates |
| | | observed | observed | observed | observed | observed |
| pH | 6.6 | 6.6 | 6.6 | 6.6 | 6.6 | 6.6 |
| Concentration | 63.6 mg/mL | 64.2 mg/mL | 64.8 mg/mL | 64.0 mg/mL | 64.9 mg/mL | 64.9 mg/mL |
| Non-reduced | Intact IgG: 96.9% | IntactIgG: 96.6% | IntactIgG: 96.2% | IntactIgG: 95.2% | IntactIgG: 94.2% | IntactIgG: 93.9% |
| and reduced | Total IgG: 96.6% | Total IgG: 96.1% | Total IgG: 95.7% | Total IgG: 95.5% | Total IgG: 93.9% | Total IgG: 94.1% |
| CE-SDS | HC: 64.5% | HC: 64.2% | HC: 64.1% | HC: 64.2% | HC: 62.9% | HC: 63.3% |
| | LC: 32.2% | LC: 31.9% | LC: 31.6% | LC: 31.3% | LC: 31.1% | LC: 30.8% |
| SEC | 97.4% Monomer | 96.8% Monomer | 96.9% Monomer | 96.4% Monomer | 96.1% Monomer | 96.3% Monomer |
| | 2.0% Aggregate | 2.4% Aggregate | 2.5% Aggregate | 2.8% Aggregate | 3.0% Aggregate | 2.9% Aggregate |
| picIEF | pI = 8.8 | pI = 8.9 | pI = 8.9 | pI = 8.9 | pI = 8.9 | pI = 8.9 |
| | Conforms to Ref | Conforms to Ref | Conforms to Ref | Conforms to Ref | Conforms to Ref | Conforms to Ref |
| | Std. No additional | Std. No additional | Std. No additional | Std. No additional | Std. No additional | Std. No additional |
| | peaks seen. | peaks seen. | peaks seen. | peaks seen. | peaks seen. | peaks seen. |
| | Main peak = | Main peak = | Main peak = | Main peak = | Main peak = | Main peak = |
| | 55.9% | 56.8% | 54.2% | 53.5% | 51.7% | 50.5% |
| | Acidic = 42.4% | Acidic = 41.2% | Acidic = 43.7% | Acidic = 44.3% | Acidic = 46.3% | Acidic = 47.4% |
| | Basic = 1.7% | Basic = 2.0% | Basic = 2.1% | Basic = 2.2% | Basic = 2.0% | Basic = 2.1% |
| Affinity | 111% | 136% | 87% | 99.7% | 130% | 97% |
| Biological | NP | NP | NP | NP | NP | 88% |
| activity | | | | | | |

NP = not performed; picIEF

TABLE 52
Stability Study: Data for Storage Conditions 25° C./60% RH

| | | Timepoints (in months) | | |
|---|---|---|---|---|
| Test | Initial | 1 | 3 | 6 |
| Appearance | Conforms | Conforms | Conforms | Confirms |
| | | No particulates observed | No particulates observed | No particulates observed |
| pH | 6.6 | 6.6 | 6.6 | 6.55 |
| Concentration | 63.6 mg/mL | 63.9 mg/mL | 64.7 mg/mL | 63.6 mg/mL |
| Non-reduced | Intact IgG: 96.9% | Intact IgG: 94.9% | Intact IgG: 90.7% | Intact IgG: 81.4% |
| and reduced | Total IgG: 96.6% | Total IgG: 94.0% | Total IgG: 90.1% | Total IgG: 84.9% |
| CE-SDS | HC: 64.5% | HC: 63.4% | HC: 62.0% | HC: 60.5% |
| | LC: 32.2% | LC: 30.6% | LC: 28.1% | LC: 24.4% |
| SEC | 97.4% Monomer | 96.2% Monomer | 95.4% Monomer | 93.3% Monomer |
| | 2.0% Aggregate | 2.8% Aggregate | 3.3% Aggregate | 4.5% Aggregate |
| icIEF | pI = 8.8 | pI = 8.9 | pI = 8.9 | pI = 8.8 |
| | Conforms to Reference | Conforms to Reference | Conforms to Reference | Conforms to Reference |
| | Standard. No additional | Standard. No additional | Standard. No additional | Standard. No additional |
| | peaks seen. | peaks seen. | peaks seen. | peaks seen. |
| | Main peak = 55.9% | Main peak = 48.6% | Main peak = 37.5% | Main peak = 25.3 |
| | Acidic = 42.4% | Acidic = 49.2% | Acidic = 60.2% | Acidic = 72.6% |
| | Basic = 1.7% | Basic = 2.2% | Basic = 2.3% | Basic = 2.1% |
| Affinity | 111% | 123% | 86% | 115% |

icIEF = Imaged capillary isoelectric focusing

TABLE 53
ICH Stability Study: Data for Storage Conditions 2-8° C., Upright

| | Timepoints (in months) | | | |
|---|---|---|---|---|
| Test | Initial | 1 | 3 | 6 |
| Appearance | Appearance conforms. | Appearance conforms. | Appearance conforms. | Appearance conforms. |
| | No particulates observed. | No particulates observed. | No particulates observed. | No particulates observed. |
| pH | 6.6 | 6.5 | 6.5 | 6.5 |
| Concentration | 61 mg/mL | 61 mg/mL | 61 mg/mL | 61 mg/mL |
| Non-reducedCE-SDS | Intact IgG: 95% | Intact IgG: 95.4% | Intact IgG: 95.2% | Intact IgG: 93% |
| ReducedCE-SDS | Total IgG: 95% | Total IgG: 95.8% | Total IgG: 95.0% | Total IgG: 94.0% |
| | Heavy Chain: 63.8% | Heavy Chain: 64.4% | Heavy Chain: 63.7% | Heavy Chain: 62.8% |
| | Light Chain: 31.6% | Light Chain: 31.4% | Light Chain: 31.3% | Light Chain: 31.6% |
| SEC | 98% Monomer | 97.1% Monomer | 97.7% Monomer | 95.8% Monomer |
| | 2% Aggregate | 2.9% Aggregate | 2.3% Aggregate | 3.9% Aggregate |
| icIEF | pI = 8.9 | pI = 8.9 | pI = 8.9 | pI = 8.9 |
| | Main peak = 55% | Main peak = 53.6% | Main peak = 52.1% | Main peak = 49.9% |
| | Acidic = 43.1% | Acidic = 44.5% | Acidic = 45.9% | Acidic = 48.2% |
| | Basic = 2.0% | Basic = 2.0% | Basic = 1.9% | Basic = 1.9% |
| Affinity | 111% | 127% | 95% | 94% |
| Biological activity | NP | NP | NP | 96% |
| Endotoxin | <0.0004 EU/mg | NP | NP | NP |
| Sterility | No growth, sterile | NP | NP | NP |
| Subvisible particulates | ≥10 μm: 15.5particles per | NP | NP | NP |
| | container; ≥25 μm: 0particles | | | |
| | percontainer | | | |
| Extractable volume | Conforms(9.3 mL) | NP | NP | Conforms (9.1 mL) |

NP = not performed

TABLE 54
ICH Stability Study: Data for Storage Conditions 25° C./60% RH, Upright

| | Timepoints (in months) | | | |
|---|---|---|---|---|
| Test | Initial | 1 | 3 | 6 |
| Appearance | Appearance conforms. | Appearance conforms. | Appearance conforms. | Appearance conforms. |
| | No particulates observed. | No particulates observed. | No particulates observed. | No particulates observed. |
| pH | 6.6 | 6.5 | 6.5 | 6.5 |
| Concentration | 61 mg/mL | 61 mg/mL | 62 mg/mL | 61 mg/mL |
| Non-reduced CE-SDS | Intact IgG: 95% | Intact IgG: 93.2% | Intact IgG: 88.1% | Intact IgG: 82% |
| Reduced CE-SDS | Total IgG: 95% | Total IgG: 94.2% | Total IgG: 88.7% | Total IgG: 84% |
| | Heavy Chain: 63.8% | Heavy Chain: 64.0% | Heavy Chain: 61.5% | Heavy Chain: 59.5% |
| | Light Chain: 31.6% | Light Chain: 30.2% | Light Chain: 27.1% | Light Chain: 24.5% |
| SEC | 98% Monomer | 96.5% Monomer | 95.2% Monomer | 92.8% Monomer |
| | 2% Aggregate | 3.5% Aggregate | 4.8% Aggregate | 3.7% Aggregate |
| icIEF | pI = 8.9 | pI = 8.9 | pI = 8.9 | pI = 8.9 |
| | Main peak = 55% | Main peak = 44.6% | Main peak = 33.9% | Main peak = 25.1% |
| | Acidic = 43.1% | Acidic = 53.2% | Acidic = 63.9% | Acidic = 73.0% |
| | Basic = 2.0% | Basic = 2.2% | Basic = 2.2% | Basic = 1.9% |
| Affinity | 149% | 95% | 91% | 96% |
| Biological activity | NP | NP | NP | 78% |
| Endotoxin | <0.0004 EU/mg | | | |
| Sterility | No growth, sterile | | | |
| Subvisible particulates | ≥10 μm: 15.5 particles per | | | ≥10 μm: 12.0 particles |
| | container; | | | per container; |
| | ≥25 μm: 0 particles per | | | ≥25 μm: 0 particles |
| | container | | | per container |
| Extractable volume | Conforms (9.3 mL) | | | Conforms (9.0 mL) |

NP = not performed

### Example 26: Results of Phase I Clinical Studies on Safety, PK, PD, and Immunogenicity

A phase I clinical study was completed to assess the safety, PK, PD, and other parameters for the anti-TL1A antibody (e.g. A219). The Phase I clinical study tested double-blind, randomized, placebo-controlled, single dose followed by multiple dose. In the Single Ascending Dose (SAD) cohort, the anti-TL1A antibody (e.g. A219) was tested in a total of 46 subjects with 3 to 1 randomization (35 to 11) in each dosing cohort. 6 cohorts (e.g. 6 dose levels) were tested for the SAD, which were 5 mg, 25 mg, 100 mg, 300 mg, 600 mg, and 1000 mg, with a follow-up period of 14 weeks. In the Multiple Ascending Dose (MAD) study, the anti-TL1A antibody (e.g. A219) was tested in 23 subjects with 3 to 1 randomization (17 to 6) in each dosing cohort. In the MAD study, all subjects received 3 doses at days 1, 15, and 29. 3 cohorts (dose levels) were tested for the MAD study, which were 50 mg, 200 mg, 500 mg, with a follow-up period of 18 weeks. The phase I clinical study evaluated the safety and tolerability, pharmacokinetics (PK), immunogenicity (e.g., by evaluating the anti-drug antibodies, ADA), and pharmacodynamic (PD) markers of the anti-TL1A antibody (e.g. A219).

68 out of the 69 (98.5%) subjects completed the study and follow-up period, with one patient completed up to Week 8 in the 200 mg MAD but lost to follow-up. No serious adverse events (SAE) were observed in the clinical study. Neither drug-related infusion reactions nor drug-related extension in infusion time was seen during the study (with 30-minute infusions of up to 1000 mg). No clinically significant changes were reported in physical exam, lab values, electrocardiogram, or vital signs.

All adverse events (AEs) assessed as related to study drug were mild. Exemplary mild AEs reported in the SAD study include 1 report of somnolence in the 35 subjects tested with A219 (at the 600 mg dose) and 1 report of headache in the 11 subject placebo group. Exemplary mild AEs reported in the MAD study include: 1 report of diarrhea in the 17 subjects tested with A219, 1 report of diarrhea in the 6 subject placebo group, 1 report of somnolence in the 17 subjects tested with A219, 1 report of dizziness in the 17 subjects tested with A219, and 1 report of headache in the 11 subject placebo group.

Therefore, the anti-TL1A antibody (e.g. A219) has favorable safety and tolerability.

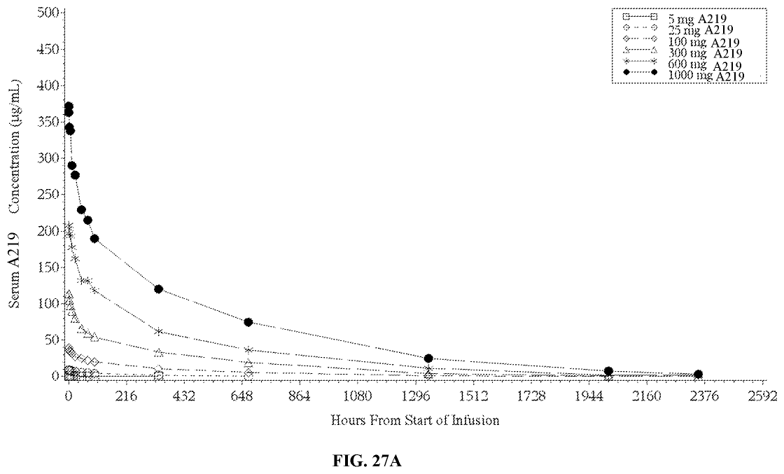

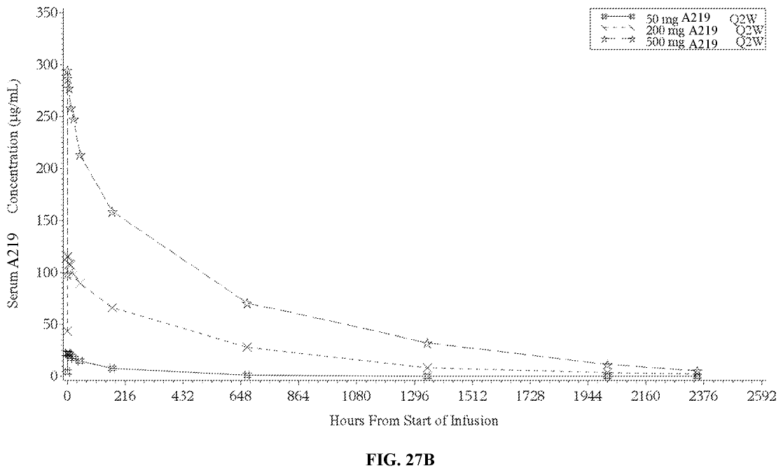

Various PK parameters were determined and shown in FIGS. 27A and 27B, and Tables 55-59.

TABLE 55
Summary of Serum A219 Pharmacokinetic Parameters Following Single Doses of A219 Administered as IV Infusion (SAD)

| Pharmacokinetic | Treatment | | | | | |
|---|---|---|---|---|---|---|
| Parameters | A | B | C | D | E | F |
| AUCₗₐₛₜ | 95.61 (79.7) | 1509 (33.1) | 10680 (16.9) | 35040 (22.8) | 72340 (24.4) | 135200 (12.5) |
| (μg*hr/mL) | [n = 6] | [n = 5] | [n = 6] | [n = 6] | [n = 6] | [n = 6] |
| AUC₁₄ | 120.6 (51.4) | 1246 (21.9) | 6080 (16.9) | 17200 (8.4) | 34940 (11.2) | 60130 (11.5) |
| (μg*hr/mL) | [n = 6] | [n = 5] | [n = 6] | [n = 6] | [n = 6] | [n = 6] |
| AUC_{inf} | 153.0 (63.4) | 1713 (33.0) | 11170 (13.9 | 35360 (23.6) | 73290 (25.6) | 137500 (12.8) |
| (μg*hr/mL) | [n = 6] | [n = 5] | [n = 6] | [n = 6] | [n = 6] | [n = 6] |
| AUC_{% extrap} (%) | 33.88 ± 20.837 | 11.41 ± 10.050 | 4.297 ± 5.5045 | 0.9035 ± 1.2359 | 1.296 ± 1.2698 | 1.730 ± 1.4655 |
| | [n = 6] | [n = 5] | [n = 6] | [n = 6] | [n = 6] | [n = 6] |
| Cₘₐₓ | 0.9647 (43.7) | 9.447 (14.8) | 39.89 (16.2) | 118.2 (18.3) | 219.7 (10.9) | 379.2 (20.6) |
| (μg/mL) | [n = 6] | [n = 5] | [n = 6] | [n = 6] | [n = 6] | [n = 6] |
| Tₘₐₓ | 0.759 (0.50, 2.00) | 1.000 (0.52, 2.00) | 0.509 (0.50, 1.00) | 0.759 (0.50, 1.00) | 1.000 (0.52, 12.00) | 0.750 (0.50, 6.00) |
| (hr) | [n = 6] | [n = 5] | [n = 6] | [n = 6] | [n = 6] | [n = 6] |
| Kₑₗ | 0.005049 ± | 0.004031 ± | 0.002710 ± | 0.002543 ± | 0.002588 ± | 0.002017 ± |
| (1/hr) | 0.0019135 | 0.0010395 | 0.00043788 | 0.00067609 | 0.0010459 | 0.00061249 |
| | [n = 6] | [n = 5] | [n = 6] | [n = 6] | [n = 6] | [n = 6] |
| t_{1/2} | 151.4 ± 46.474 | 181.4 ± 46.482 | 262.4 ± 49.651 | 296.1 ± 109.39 | 306.5 ± 116.50 | 369.7 ± 108.15 |
| (hr) | [n = 6] | [n = 5] | [n = 6] | [n = 6] | [n = 6] | [n = 6] |
| CL (L/hr) | 37.48 ± 21.666 | 15.17 ± 4.3784 | 9.024 ± 1.3063 | 8.676 ± 1.9984 | 8.406 ± 2.1096 | 7.322 ± 1.0102 |
| | [n = 6] | [n = 5] | [n = 6] | [n = 6] | [n = 6] | [n = 6] |
| V_{z} | 7.142 ± 2.1467 | 3.762 ± 0.64753 | 3.427 ± 0.82422 | 3.555 ± 0.88397 | 3.485 ± 0.85549 | 3.846 ± 1.0698 |
| (L) | [n = 6] | [n = 5] | [n = 6] | [n = 6] | [n = 6] | [n = 6] |
| DN AUCₗₐₛₜ | 19.12 (79.7) | 60.37 (33.1) | 106.8 (16.9) | 116.8 (22.8) | 120.6 (24.4) | 135.2 (12.5) |
| (μg*hr/mL/mg) | [n = 6] | [n = 5] | [n = 6] | [n = 6] | [n = 6] | [n = 6] |
| DN AUC₁₄ | 2.547 (1094.4) | 16.79 (602.2) | 53.11 (881.3) | 150.2 (760.8) | 342.6 (618.7) | 104.6 (98.2) |
| (μg*hr/mL/mg) | [n = 6] | [n = 5] | [n = 6] | [n = 6] | [n = 6] | [n = 6] |
| DN AUC_{inf} | 30.60 (63.4) | 68.52 (33.0) | 111.7 (13.9) | 117.9 (23.6) | 122.1 (25.6) | 137.5 (12.8) |
| (μg*hr/mL/mg) | [n = 6] | [n = 5] | [n = 6] | [n = 6] | [n = 6] | [n = 6] |
| DN Cₘₐₓ | 0.1929 (43.7) | 0.3779 (14.8) | 0.3989 (16.2) | 0.3940 (18.3) | 0.3662 (10.9) | 0.3792 (20.6) |
| (μg/mL/mg) | [n = 6] | [n = 5] | [n = 6] | [n = 6] | [n = 6] | [n = 6] |

A single dose of the treatments was administered as an IV infusion over 30 minutes at Hour 0 on Day 1.
Treatment:
A = 5 mg A219;
B = 25 mg A219;
C = 100 mg A219;
D = 300 mg A219;
E = 600 mg A219;
F = 1000 mg A219
AUCs and Cₘₐₓ are presented as geometric mean and geometric CV %.
Tₘₐₓ values are presented as median (minimum, maximum).
Other parameters are presented as arithmetic mean (±SD).

TABLE 56
Summary of Serum A219 Pharmacokinetic Parameters Following Multiple
Doses of A219 Q2W Administered as IV Infusion - Day 1 (MAD)

| Pharmacokinetic | Treatment | | |
|---|---|---|---|
| Parameters | G | H | I |
| AUCₗₐₛₜ (μg*hr/mL) | 2640 (14.2) [n = 6] | 11600 (12.3) [n = 6] | 30760 (16.6) [n = 5] |
| AUC₁₄ (μg*hr/mL) | 2640 (14.2) [n = 6] | 11610 (12.3) [n = 6] | 30770 (16.6) [n = 5] |
| Cₘₐₓ (μg/mL) | 17.92 (12.4) [n = 6] | 73.77 (15.7) [n = 6] | 199.0 (17.6) [n = 5] |
| Tₘₐₓ (hr) | 0.759 (0.52, 1.12) [n = 6] | 1.500 (1.00, 6.00) [n = 6] | 1.000 (1.00, 1.05) [n = 5] |
| C_{trough} (μg/mL) | 4.070 ± 1.2370 [n = 6] | 22.55 ± 3.4233 [n = 6] | 59.40 ± 8.6232 [n = 5] |
| Kₑₗ (1/hr) | 0.003658 ± 0.0013710 [n = 6] | 0.002395 ± 0.00033531 [n = 6] | 0.002401 ± 0.00034915 [n = 5] |
| t_{1/2} (hr) | 211.3 ± 71.445 [n = 6] | 293.6 ± 35.985 [n = 6] | 293.5 ± 41.107 [n = 5] |
| DN AUCₗₐₛₜ (μg*hr/mL/mg) | 52.80 (14.2) [n = 6] | 58.01 (12.3) [n = 6] | 61.52 (16.6) [n = 5] |
| DN AUC₁₄ (μg*hr/mL/mg) | 22.67 (139.1) [n = 6] | 85.51 (167.1) [n = 6] | 106.6 (59.6) [n = 5] |
| DN Cₘₐₓ (μg/mL/mg) | 0.3584 (12.4) [n = 6] | 0.3688 (15.7) [n = 6] | 0.3980 (17.6) [n = 5] |

Multiple doses of the treatments were administered as an IV infusion over 30 minutes at Hour 0 on Days 1, 15, and 29.
Treatment: G = 50 mg A219 Q2W; H = 200 mg A219 Q2W; I = 500 mg A219 Q2W
AUCs and Cₘₐₓ are presented as geometric mean and geometric CV %.
Tₘₐₓ values are presented as median (minimum, maximum).
Other parameters are presented as arithmetic mean (±SD).

TABLE 57
Summary of Serum A219 Pharmacokinetic Parameters Following Multiple
Doses of A219 Q2W Administered as IV Infusion - Day 15 (MAD)

| Pharmacokinetic | Treatment | | |
|---|---|---|---|
| Parameters | G | H | I |
| AUCₜₐᵤ (μg*hr/mL) | 3407 (29.1) [n = 6] | 19810 (15.9) [n = 6] | 50020 (15.4) [n = 5] |
| C_{max, ss} (μg/mL) | 23.37 (18.0) [n = 6] | 99.02 (15.9) [n = 6] | 255.7 (17.6) [n = 5] |
| C_{min, ss} (μg/mL) | 5.215 ± 3.0633 [n = 6] | 43.52 ± 6.9761 [n = 6] | 117.8 ± 50.064 [n = 5] |
| C_{av, ss} (μg/mL) | 10.46 ± 2.5815 [n = 6] | 59.58 ± 9.5902 [n = 6] | 150.3 ± 23.364 [n = 5] |
| C_{trough} (μg/mL) | 5.215 ± 3.0633 [n = 6] | 43.52 ± 6.9761 [n = 6] | 99.56 ± 18.158 [n = 5] |
| T_{max, ss} (hr) | 2.067 (0.52, 6.00) [n = 6] | 0.534 (0.50, 6.08) [n = 6] | 2.000 (0.60, 2.40) [n = 5] |
| Kₑₗ (1/hr) | 0.004610 ± 0.0024974 [n = 6] | 0.001823 ± 0.00024889 [n = 6] | 0.002296 ± 0.00054397 [n = 5] |
| t_{1/2} (hr) | 191.3 ± 95.015 [n = 6] | 386.4 ± 55.219 [n = 6] | 313.6 ± 62.774 [n = 5] |
| CLₛₛ (L/hr) | 15.23 ± 4.9675 [n = 6] | 10.20 ± 1.5635 [n = 6] | 10.09 ± 1.5138 [n = 5] |
| Vₛₛ (L) | 3.790 ± 1.3556 [n = 6] | 5.675 ± 1.1080 [n = 6] | 4.604 ± 1.3739 [n = 5] |
| DN AUCₜₐᵤ (μg*hr/mL) | 68.15 (29.1) [n = 6] | 99.05 (15.9) [n = 6] | 100.0 (15.4) [n = 5] |
| DN C_{max, ss} (μg/mL) | 0.4674 (18.0) [n = 6] | 0.4951 (15.9) [n = 6] | 0.5115 (17.6) [n = 5] |
| R_{ac, AUC} | 1.315 ± 0.29105 [n = 6] | 1.717 ± 0.20859 [n = 6] | 1.630 ± 0.12819 [n = 5] |
| R_{ac, Cmax} | 1.309 ± 0.12730 [n = 6] | 1.344 ± 0.069527 [n = 6] | 1.288 ± 0.091554 [n = 5] |
| RP-T | 7.114 ± 5.4916 [n = 6] | 2.320 ± 0.33489 [n = 6] | 2.413 ± 0.78742 [n = 5] |
| % FLUC | 188.8 ± 71.269 [n = 6] | 95.92 ± 22.110 [n = 6] | 94.78 ± 33.105 [n = 5] |

Multiple doses of the treatments were administered as an IV infusion over 30 minutes at Hour 0 on Days 1, 15, and 29.
Treatment: G = 50 mg A219 Q2W; H = 200 mg A219 Q2W; I = 500 mg A219 Q2W
AUCs and Cₘₐₓ are presented as geometric mean and geometric CV %.
Tₘₐₓ values are presented as median (minimum, maximum).
Other parameters are presented as arithmetic mean (±SD).

TABLE 58
Summary of Serum A219 Pharmacokinetic Parameters Following Multiple
Doses of A219 Q2W Administered as IV Infusion - Day 29 (MAD)

| Pharmacokinetic | Treatment | | |
|---|---|---|---|
| Parameters | G | H | I |
| AUCₜₐᵤ (μg*hr/mL) | 3148 (51.4) [n = 6] | 23740 (11.9) [n = 6] | 57430 (10.6) [n = 5] |
| C_{max, ss} (μg/mL) | 23.08 (19.5) [n = 6] | 119.5 (11.5) [n = 6] | 300.1 (13.9) [n = 5] |
| C_{min, ss} (μg/mL) | 9.190 ± 4.9563 [n = 6] | 66.30 ± 6.8972 [n = 6] | 159.4 ± 15.821 [n = 5] |
| C_{av, ss} (μg/mL) | 10.22 ± 4.1844 [n = 6] | 71.08 ± 8.5764 [n = 6] | 171.7 ± 18.425 [n = 5] |
| C_{trough} (μg/mL) | 0.6997 ± 0.63205 [n = 6] | 16.93 ± 6.9592 [n = 6] | 47.83 ± 10.898 [n = 5] |
| T_{max, ss} (hr) | 1.325 (0.52, 5.95) [n = 6] | 2.000 (0.50, 6.13) [n = 6] | 2.000 (0.50, 6.00) [n = 5] |
| Kₑₗ (1/hr) | 0.003935 ± 0.0031363 [n = 6] | 0.001901 ± 0.00071255 [n = 6] | 0.001677 ± 0.00067543 [n = 5] |
| t_{1/2} (hr) | 272.6 ± 195.15 [n = 6] | 398.5 ± 112.66 [n = 6] | 459.2 ± 148.22 [n = 5] |
| CLₛₛ (L/hr) | 17.65 ± 9.6843 [n = 6] | 8.473 ± 0.98033 [n = 6] | 8.744 ± 0.90047 [n = 5] |
| Vₛₛ (L) | 5.427 ± 2.4446 [n = 6] | 4.788 ± 1.1813 [n = 6] | 5.816 ± 2.1556 [n = 5] |
| DN AUCₜₐᵤ | 62.97 (51.4) [n = 6] | 118.7 (11.9) [n = 6] | 114.9 (10.6) [n = 5] |
| (μg*hr/mL) | | | |
| DN C_{max, ss} (μg/mL) | 0.4616 (19.5) [n = 6] | 0.5975 (11.5) [n = 6] | 0.6002 (13.9) [n = 5] |
| R_{ac, AUC} | 1.278 ± 0.51418 [n = 6] | 2.050 ± 0.14354 [n = 6] | 1.872 ± 0.16208 [n = 5] |
| R_{ac, Cmax} | 1.297 ± 0.16900 [n = 6] | 1.630 ± 0.19500 [n = 6] | 1.511 ± 0.10221 [n = 5] |
| RP-T | 3.381 ± 1.9300 [n = 6] | 1.814 ± 0.11010 [n = 6] | 1.897 ± 0.18838 [n = 5] |
| % FLUC | 174.3 ± 107.19 [n = 6] | 75.58 ± 5.6244 [n = 6] | 83.10 ± 15.890 [n = 5] |

Multiple doses of the treatments were administered as an IVinfusion over 30 minutes at Hour 0 on Days 1, 15, and 29.
Treatment: G = 50 mg A219 Q2W; H = 200 mg A219 Q2W; I = 500 mg A219 Q2W
AUCs and Cₘₐₓ are presented as geometric mean and geometric CV %.
Tₘₐₓ values are presented as median (minimum, maximum).
Other parameters are presented as arithmetic mean (±SD).

TABLE 59
Steady-State Assessment of Serum A219 C_{trough} Values Following
Multiple Doses of A219 Q2W Administered as IV Infusion (MAD)

| Treatment | Day | Geometric LS Mean | p-value |
|---|---|---|---|
| G | C_{trough} Day 1 | 3.897 | 0.0952 |
| | | 4.180 | 0.0075 |
| | C_{trough} Day 2 | 0.189 | . |
| H | C_{trough} Day 1 | 22.34 | 0.4891 |
| | | 43.08 | 0.0004 |
| | C_{trough} Day 2 | 14.88 | . |
| I | C_{trough} Day 1 | 58.91 | 0.1052 |
| | | 98.26 | <0.0001 |
| | C_{trough} Day 2 | 46.84 | . |

Multiple doses of the treatments were administered as an IV infusion over 30 minutes at Hour 0 on Days 1, 15, and 29.
Treatment: G = 50 mg A219 Q2W; H = 200 mg A219 Q2W; I = 500 mg A219 Q2W
Concentrations were In-transformed prior to analysis.
Geometric least-squares means (LSMs) were obtained by taking the exponential of the LSMs from ANOVA.
p-value corresponds to the Helmert contrast, i.e., the comparison of that day versus the average of the remaining days.
The C_{trough} values on Days 1, 15, and 29 correspond to the predose on Days 15, and 29, as well as the derived C_{trough} following dosing on Day 29, respectively.
. = Value missing or not reportable.

Results shown in FIGS. 27A and 27B, and Tables 55-59 demonstrate that the PK of the anti-TL1A antibody (e.g. A219) meets the PK performance standards for a therapeutic antibody and supports the phase 2 dosing regimen discussed in Section 5 (Examples). The half-life after a dose of 500 mg every other week is about 19 days. Dose proportional exposure at doses greater than or equal to 100 mg was observed in the PK profile.

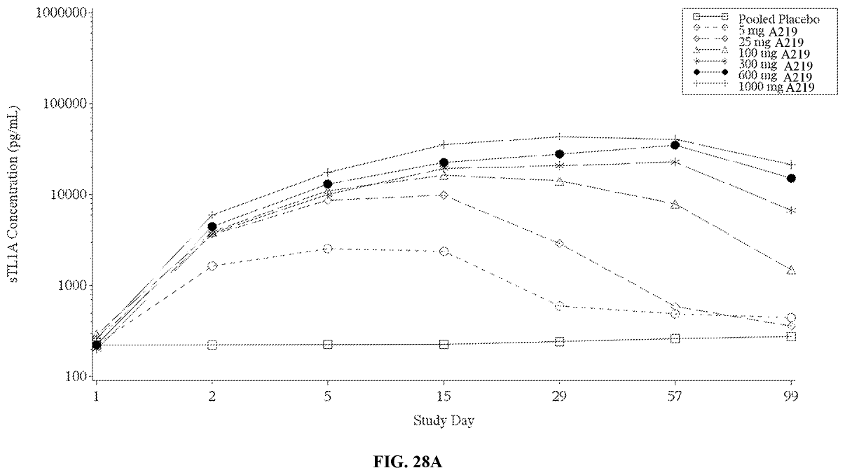

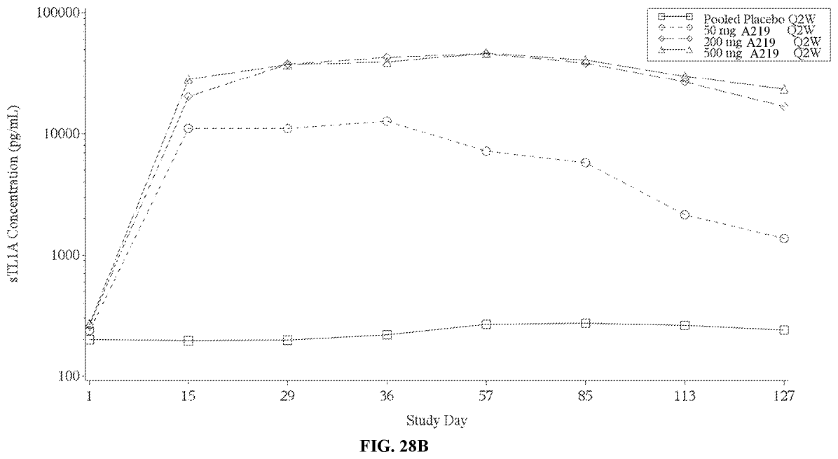

Furthermore, target engagement was assessed by determining the soluble TL1A concentration in the serum of the subjects in the clinical studies. The anti-TL1A antibody A219 provided herein demonstrated dose-dependent, robust, sustained target engagement as shown in FIGS. 28A and 28B. Target engagement as determined by the increase in the soluble TL1A in serum maximized at 200 mg A219 Q2W at about 45,000 μg/mL sTL1A (FIG. 28B). Such a target engagement is more than 4 fold higher than observed in the control reference anti-TL1A antibody that only binds to trimeric TL1A (control reference antibody sequence, light chain SEQ ID NO: 382 and heavy chain SEQ ID NO: 383) (see Banfield C, et al. Br J Clin Pharmacol. 2020; 86:812-824; Danese S, et al. Clin Gastroenterol Hepatol. 2021 Jun. 11; S1542-3565 (21) 00614-5; Danese S, et al. Clin Gastroenterol Hepatol. 2021 November; 19 (11): 2324-2332.e6). Therefore, the anti-TL1A antibody provided herein that binds to both monomeric and trimeric TL1A provide superior target engagement over the anti-TL1A antibody that binds to only trimeric TL1A.

Additionally, immunogenicity of the anti-TL1A antibody is assessed by determining the anti-drug antibody (ADA). At clinically relevant dose (1000 mg SAD, 200 mg and 500 mg MAD), immunogenicity rate was no more than 20%. In contrast, the reported immunogenicity (e.g., ADA positivity) rate for the control reference anti-TL1A antibody (light chain SEQ ID NO: 382 and heavy chain SEQ ID NO: 383) was over 80% at similar doses in both normal healthy volunteer and UC patients (see Banfield C, et al. Br J Clin Pharmacol. 2020; 86:812-824; Danese S, et al. Clin Gastroenterol Hepatol. 2021 Jun. 11; S1542-3565 (21) 00614-5; Danese S, et al. Clin Gastroenterol Hepatol. 2021 November; 19 (11): 2324-2332.e6). ADA titers observed in the clinical trial were inversely proportional to A219 exposure and ADA positivity only occurred at low A219 concentrations.

To further evaluate the potential impact of ADA, neutralizing antibody was also determined in the phase I trial. Neutralizing antibody rate at clinically relevant dose was uncommon and was observed only in 1 out of 17 subjects (6%) across the clinically relevant dose groups (1000 mg SAD, 200 mg and 500 mg MAD). Immunogenicity observed in the phase I trial was not clinically relevant in that (1) ADA did not impact safety because there were no report of infusion reaction throughout the study, (2) ADA did not impact clearance of A219 in the population PK model, and (3) ADA did not impact target engagement because there was no apparent impact on sTL1A level as shown in FIGS. 28A and 28B.

In summary, the anti-TL1A antibody provided herein has favorable safety and tolerability; PK of the anti-TL1A antibody provided herein meets performance standards and supports phase 2 dosing regimen; the anti-TL1A antibody provided herein neutralizes both active trimeric TL1A and inactive monomeric TL1A, leading to increased and sustained target engagement and potentially to more effective reduction of active TL1A in tissues; the anti-TL1A antibody provided herein do not trigger immunogenicity that may adversely impact its therapeutic efficacy.

### Example 27: Further Validation of Physiologically Based Pharmacokinetic (PBPK) Modeling and Population Pharmacokinetic Modeling (popPK) with the Phase I Clinical Trial Results

Based on the PK, PD, and TL1A concentration data from the subjects of the phase I clinical trial, further PBPK modeling, popPK modeling, and validation of the models were conducted. As further described above (e.g. in this Section 5 (Examples)), the key mechanisms included in the PBPK models include: central, peripheral, and diseased tissue (e.g. gut) compartment; TL1A synthesis and clearance, interchange between trimer and monomer states; upregulated TL1A synthesis in disease gut tissue of IBD patients; binding by A219 to both monomer and trimer TL1A and binding a control reference antibody only to trimer TL1A; administration, distribution, non-specific elimination, and membrane TL1A mediated target mediated drug disposition for the anti-TL1A antibodies; distribution and clearance of bound complexes. The inputs to the PBPK model include: (1) the anti-TL1A antibody provided herein binds to both TL1A monomer and trimer, whereas the control reference antibody (light chain SEQ ID NO: 382 and heavy chain SEQ ID NO: 383) binds only to TL1A trimer; (2) TL1A is synthesized systemically in the peripheral compartment in healthy subjects and in inflamed gut tissue, and the elevated tissue expression of TL1A is caused by increased synthesis of TL1A within the diseased tissue; (3) trimer TL1A and monomer TL1A interchange at a rapid equilibrium, resulting in a fixed steady state ratio of monomer to trimer; (4) TL1A trimers/monomers bound to drug do not change forms; (5) the anti-TL1A antibody binds trimer or monomer with the same effective Kd in a single binding event; (6) free TL1A monomer and trimer clear at different rates; (7) antibody bound TL1A monomer and antibody bound TL1A trimer clear at the same rate; (8) antibody bound TL1A monomer and antibody bound TL1A trimer distribute the same as the antibody; antibody bound to membrane TL1A internalizes at the same rate as membrane TL1A. The exemplary values for the various parameters for the anti-TL1A antibodies are described in Table 60.

TABLE 60
Parameters used in the modeling (drug = anti-TL1A antibody)

| Value | Description |
|---|---|
| 3 | Circulation volume |
| 13 | Systemic interstitial volume |
| 0.27 (6.6 h) | Half-life of free TL1A trimer |
| 0.028 (40 min) | Half-life of free TL1A monomer |
| 0.6 | $Mass fraction of trimer \frac{trimer}{trimer + monomer}$ |
| 1.9 | Distribution timescale for TL1A into the peripheral |
| | compartment |
| 1 | Partition coefficient of TL1A into the peripheral |
| | compartment |
| 0.1 | Total membrane bound TL1A (membrane TMDD) |
| 6 | Internalization rate of free receptor |
| 144 | Drug molecular weight (for dosing) |
| 2.5 | Absorption half-time (typical mAb) for SC dosing |
| 0.5 | Bioavailability for SC dosing |
| 0.05 | Drug binding affinity to TL1A |
| 1 | Ability of drug to bind TL1A monomer |
| 21 | Linear elimination half-life of drug |
| 35 | Distribution timescale for drug into peripheral |
| | compartment |
| 0.2 | Partition coefficient of drug into peripheral compart- |
| | ment |
| 35 | Distribution timescale for drug: TL1A complex into |
| | peripheral compartment |
| 0.2 | Partition coefficient of drug: TL1A complex into the |
| | peripheral compartment |
| 1.1 | Linear elimination half-life of drug: trimer complex |
| 1.1 | Linear elimination half-life of drug: monomer complex |
| 225 | Baseline concentration of total TL1A |
| 12.95 | Systemic interstitial volume minus diseased gut volume |
| 0.05 | Diseased gut volume assuming 50% colon involvement |
| | with 100 mL colon interstitial volume (Shah and Betts) |
| 20 | Fold molar increase in TL1A synthesis rate in the |
| | diseased gut tissue compared to the peripheral synthesis |
| | rate |
| 35 | Distribution timescale for drug into gut compartment |
| 0.04 | Partition coefficient of drug into gut compartment |
| 35 | Distribution timescale for drug: TL1A complex into |
| | gut compartment |
| 0.04 | Partition coefficient of drug: TL1A complex into the |
| | gut compartment |
| 225 | Baseline concentration of total TL1A (disease) |

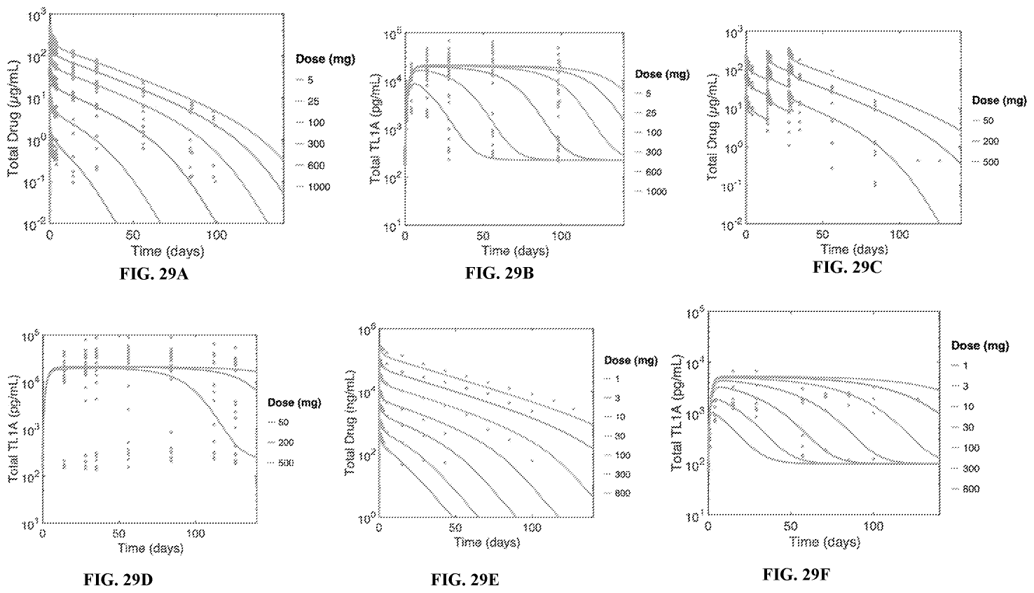

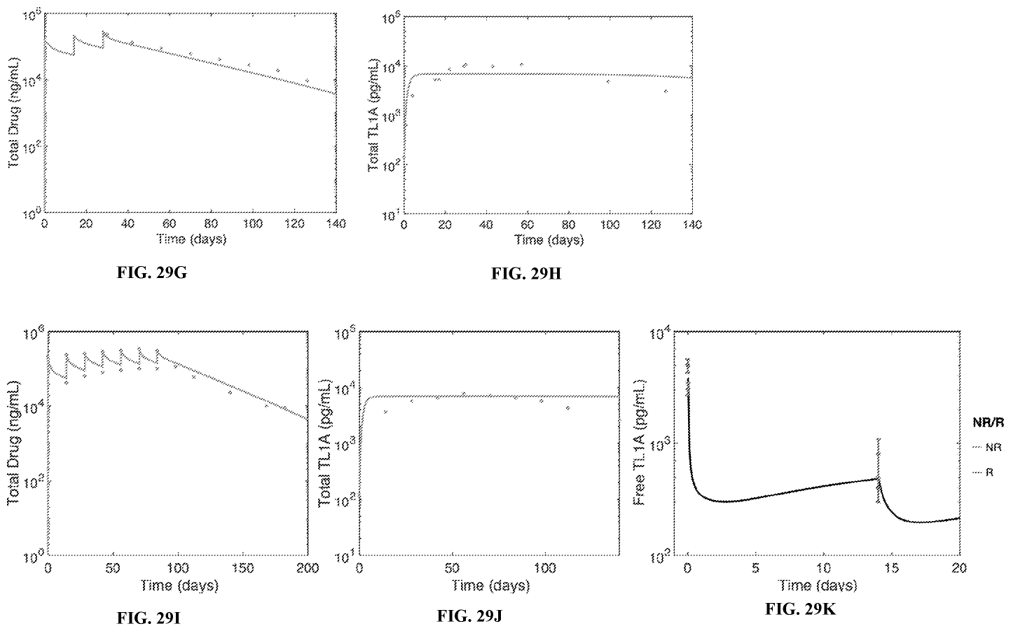

For validation of the model, the model was fit to the SAD data and benchmarked to Q2W data of the phase I clinical trial with A219. As shown in FIGS. 29A and 29B, the model fitted to single ascending dose data of A219 with reasonable agreement. Furthermore, as shown in FIGS. 29C and 29D, the model was able to capture multiple ascending dose data of A219 without additional fitting, indicating the consistency and robustness of the model. Similarly and without additional fitting, the model captured the data of a control reference antibody that binds only to TL1A trimer (light chain SEQ ID NO: 382 and heavy chain SEQ ID NO: 383) with regard to (1) phase I single ascending dose data (FIGS. 29E and 29F), (2) phase I multiple ascending dose data (FIGS. 29G and 29H), and (3) phase II data on PK & total STL1A levels (FIGS. 291 and 29J) (Banfield C, et al. Br J Clin Pharmacol. 2020; 86:812-824. Danese S, et al., Clin Gastroenterol Hepatol. 2021 November; 19 (11): 2324-2332.e6, Hassan-Zahraee M, et al. Inflammatory Bowel Diseases 2021, XX, 1-13). The IBD specific parameters were then calibrated to capture free tissue TL1A levels in the gut (FIG. 29K) as observed with the control reference antibody (light chain SEQ ID NO: 382 and heavy chain SEQ ID NO: 383) in clinical trials. As such the model was validated with the clinical trial data.

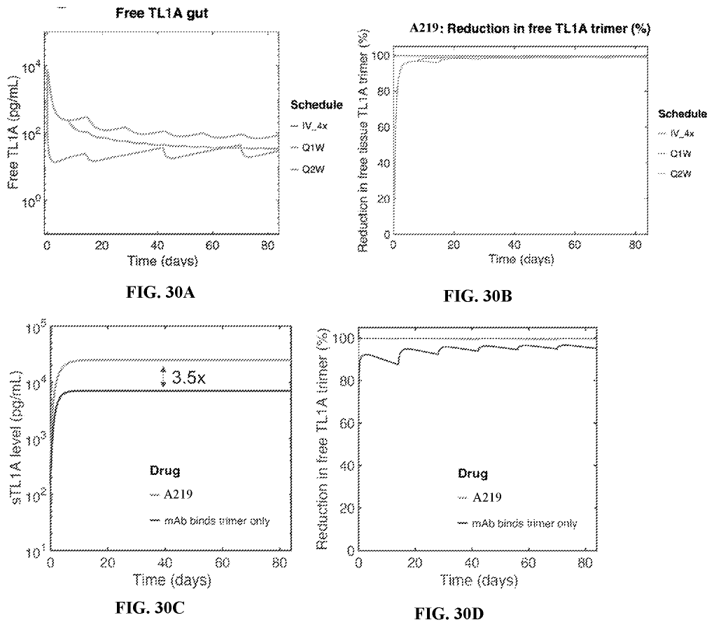

This validated model can be used to determine the dose to reduce the free TL1A concentration in the patient's diseased tissue to below the TL1A concentration of the corresponding tissue in a healthy subject, similar to that described above in Example 20 and 21. FIGS. 30A and 30B show examples of such doses determined from the validated model that can bring the free TL1A concentration in the patient's diseased tissue to below the TL1A concentration of the corresponding tissue in a healthy subject (IV_4×=1000 mg loading dose, 3×500 mg on days 14, 42, 70; SC dosing 240 mg Q1W or Q2W).

The validated model also confirmed that anti-TL1A antibodies that bind to both TL1A monomer and trimer can engage more TL1A in circulation and result in greater reduction of TL1A in diseased tissue than anti-TL1A antibodies that only bind to TL1A trimer. In a head-to-head comparison in the validated model, anti-TL1A antibodies that bind to both TL1A monomer and trimer engaged more TL1A in circulation than anti-TL1A antibodies that only bind to TL1A trimer as shown in FIG. 30C, with the circulation TL1A accumulation ratio determined to be 3.5 fold. In a head-to-head comparison in the validated model, anti-TL1A antibodies that bind to both TL1A monomer and trimer also resulted in higher percentage of TL1A reduction of TL1A in diseased tissue (about 100% reduction from day 0) when compared to anti-TL1A antibodies that only bind to TL1A trimer, as shown in FIG. 30D. Because the diseased tissue of IBD patients often produces 20, 30, 40, 50, 60, 70, or even 100 fold more TL1A as shown in Examples 20 and 21 above, a few percentage point of residual TL1A production in the diseased tissue of the patients can still be a pathological TL1A concentration.

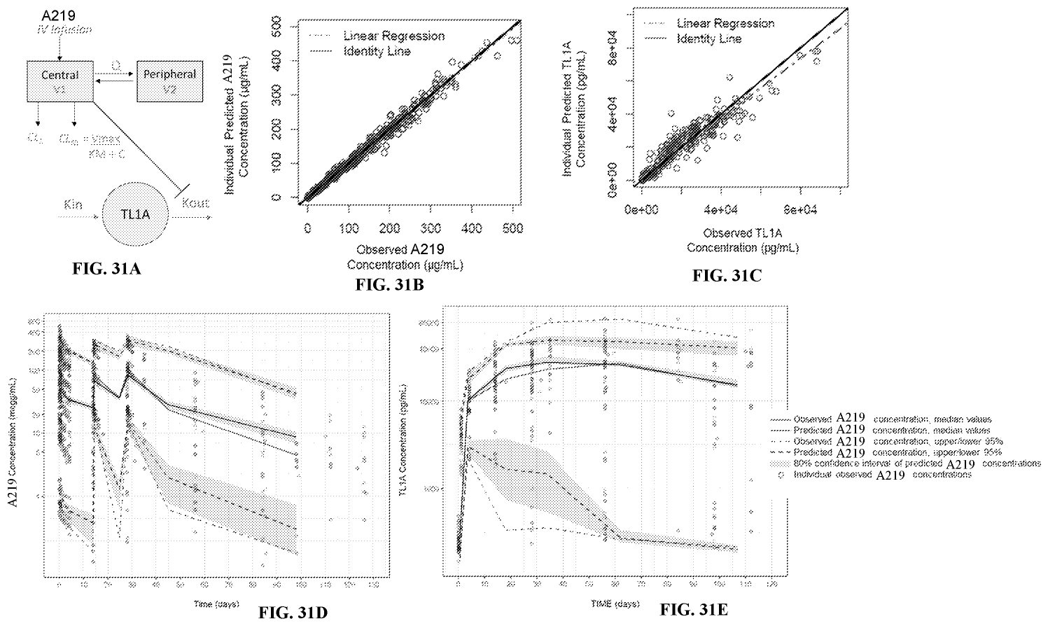

Similarly, popPK model was further fitted and validated with the phase I clinical trial data. Briefly, 2 compartment popPK Model for A219 with linear and non-linear elimination (target-mediated drug disposition) was established as shown in FIG. 31A and as described above. No covariates were found to have a clinically relevant effect on PK parameters. The popPK model fitted the phase I clinical trial data well and reliably predicted A219 and TL1A concentration data in the tested population, as shown in FIGS. 31B-31E. Further, there was no apparent bias between the predicted and observed A219 and TL1A concentrations (FIGS. 31B-31E).

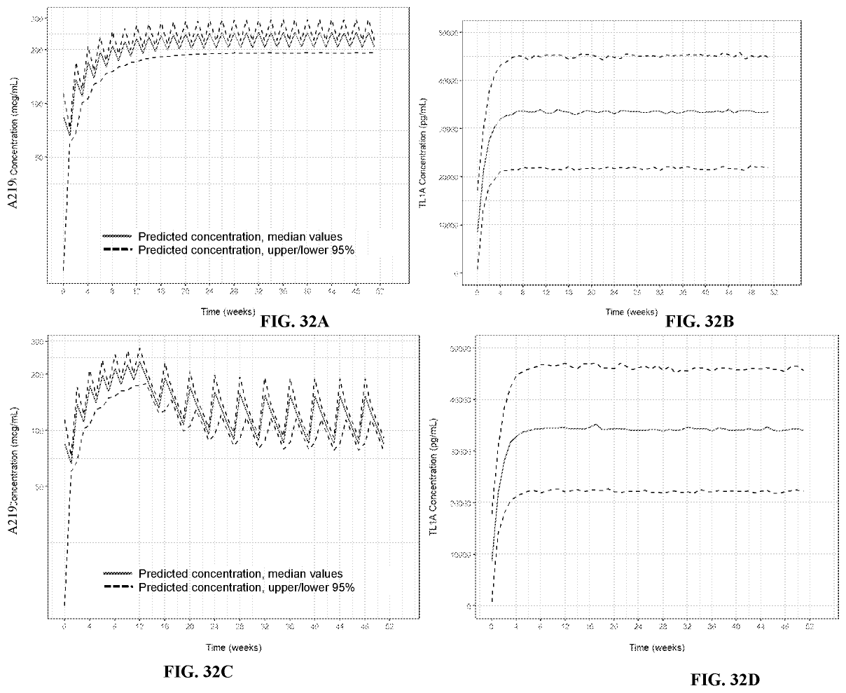

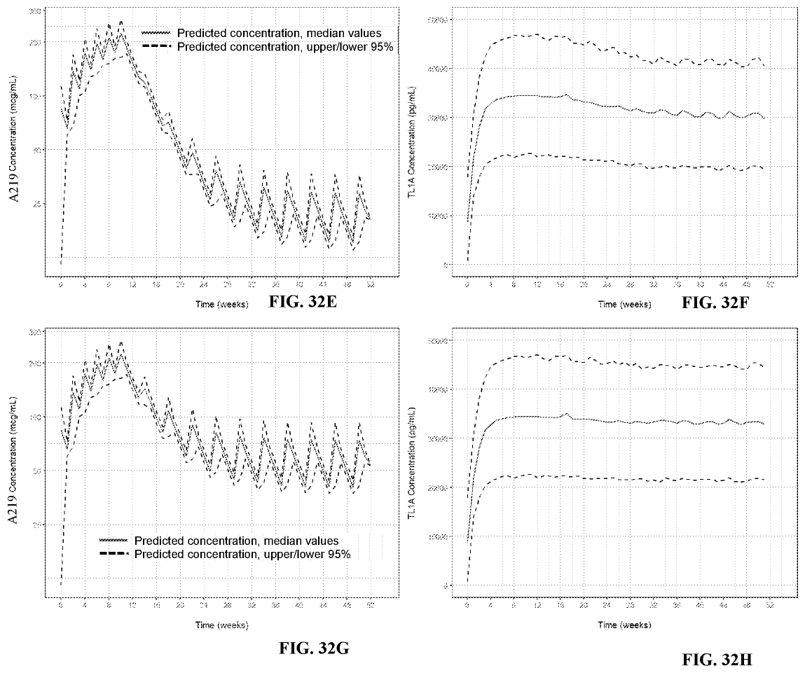

Having validated the popPK model, the popPK model was used to determine A219 and TL1A concentrations under various dosing regimen. The validated popPK model confirmed the dose to achieve the levels of anti-TL1A antibody concentration and engagement of TL1A in the serum (total soluble TL1A concentration in circulation) in order to lower the TL1A concentration in the diseased tissue to below that of a healthy subject, as shown in FIGS. 32A-32H.

### Example 28: Gene Expression Analysis of Single Cell RNAseq Data from IBD Tissues Provides a Rationale for Blockade of TL1A/DR3 and IL23/IL23R Pathways in Combination as a Novel Therapeutic Approach to Treat IBD

The present study shows gene expression at the single cell level (e.g. single cell RNAseq) from IBD patient gut tissue biopsies and reveals that expression of TL1A/DR3 and IL23/IL23R genes is present in various immune and stromal cell types. Expression of these pathways signaling receptors can be seen in some of the same cell types as well as in different cell types. Without being bound by the theory, the disclosure thus provides that in instances when a particular cell type expresses both pathway receptors, blockade of both is needed in order to effectively neutralize the pro-inflammatory function of that cell. Additionally, cells expressing either pathway alone can also contribute to inflammation and disease independently and blocking either pathway alone can leave a significant population of inflammation-causing immune cells unchecked. Thus, the disclosure provides that combining therapeutics to block both TL1A/DR3 and IL23/IL23R pathways can be more efficacious than blocking either pathway alone. Combining therapeutics to block both TL1A/DR3 and IL23/IL23R pathways can result in blockade of multiple inflammation-causing cell types and result in a synergistic reduction in inflammation.

In this example, single cell RNAseq profiles of colonic biopsies from 18 ulcerative colitis patients and 12 healthy subjects (Smillie C S, Biton M, Ordovas-Montanes J, et. al., Intra- and Inter-cellular Rewiring of the Human Colon during Ulcerative Colitis. Cell. 2019 Jul. 25; 178 (3): 714-730.e22. doi: 10.1016/j.cell.2019.06.029) were analyzed for gene expression. Briefly, lamina propria cells were partitioned into immune or stromal compartments, followed by cell clustering and lineage identification. T-Stochastic Neighborhood Embedding (t-SNE) was used to visualize either cells colored by cell subset or normalized gene expression.

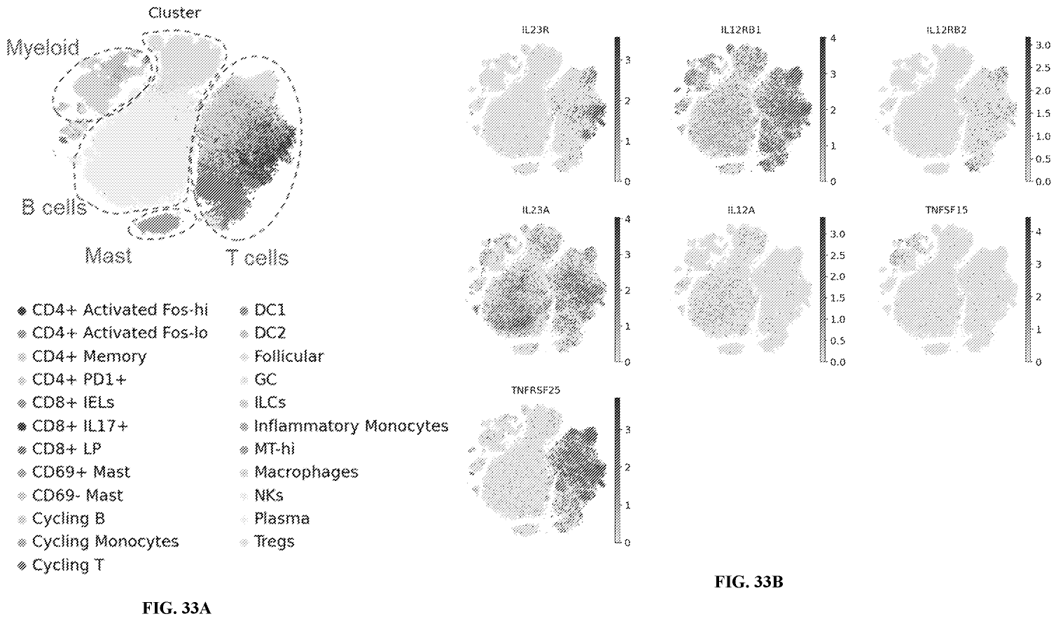

FIGS. 33A-33B shows gene expression analysis in immune cells from IBD and healthy control biopsies. FIG. 33A shows single cell RNAseq data clustered based on gene expression at the single cell level and identifies major immune cells clusters and subsets. In FIG. 33B, cells expressing IL23A (IL23), IL12A (IL12), IL23R (IL23R, IL12RB1), IL12R (IL12RB2, IL12RB1), TL1A (TNFSF15) and DR3 (TNFRSF25) were shown and were colocalized to immune cell subsets expressing them with correspondence to FIG. 33A. Based on the comparison between FIG. 33A and FIG. 33B, IL23A was expressed in T cells, myeloid and B cells; IL12A was expressed in B cells, IL23R was expressed in T cells and innate lymphoid cells (ILCs); IL12RB1 was expressed ubiquitously; IL12RB2 was expressed in T cells and NKs (natural killer cells); TNFSF15 was expressed in myeloid cells and TNFRSF25 was expressed in T cells and ILCs.

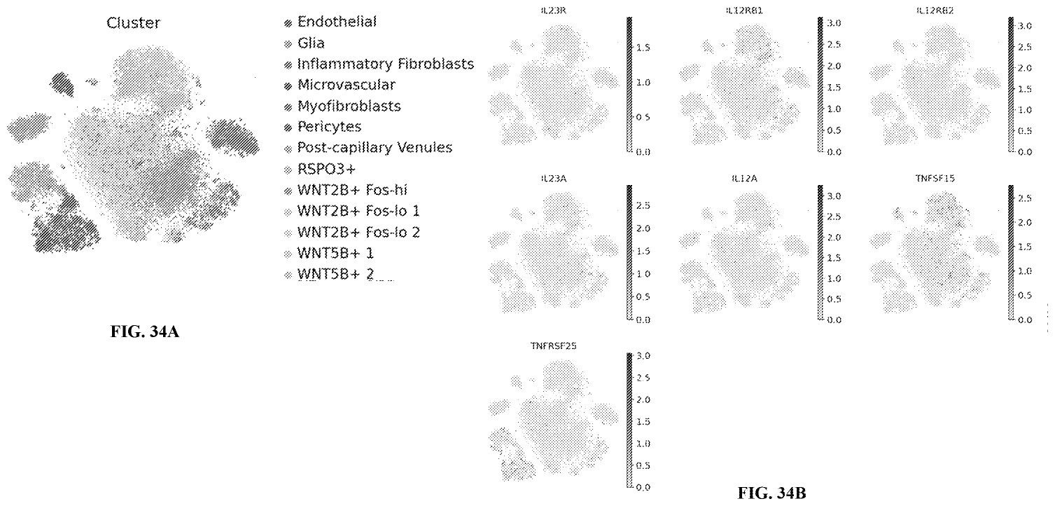

FIGS. 34A-34B shows gene expression analysis in stromal cells from IBD biopsies and healthy controls. FIG. 34A shows single cell RNAseq data clustered based on gene expression at the single cell level and identifies major stromal cell clusters and subsets. In FIG. 34B, cells expressing TL1A (TNFSF15), DR3 (TNFRSF25), IL23A, IL12A, IL23R, IL12RB1 and IL12RB2 were shown and were colocalized to stromal cell subsets expressing them with correspondence to FIG. 34A.

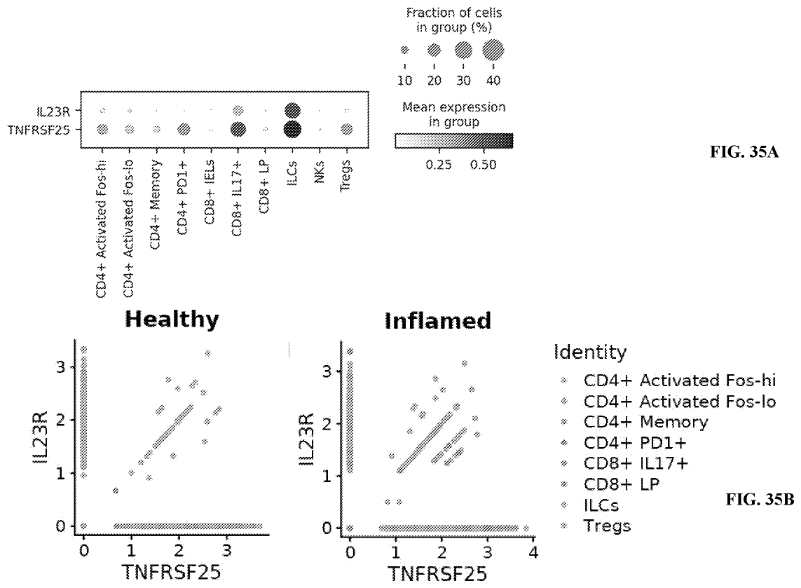

Gene expression of TNFRSF15 (DR3) and IL23R in various immune cells from IBD biopsies was analyzed and shown in FIG. 35A. CD4+ and CD8+ inflammatory T cells as well as ILCs expressed DR3 and IL23R. T cells and ILCs expressed high levels of DR3 and lower levels of IL23R.

Additionally, co-expression analysis at single cell level for IL23R and DR3 was performed and shown in FIG. 35B. Inflamed tissue from IBD subjects had increased co-expression of IL23R and DR3 in T cells as shown in FIG. 35B.

Accordingly, certain inflammatory immune cells expressed both TL1A and IL23 pathway receptors, blockade of both are needed in order to completely neutralize the pro inflammatory function of such inflammatory cells. Additionally, certain inflammatory immune cells expressed either pathway alone and can also contribute to inflammation and disease independently. Blocking both pathways are beneficial to inhibit multiple and larger populations of inflammatory immune cells to achieve synergistic efficacy over individual blockade alone.

### Example 29: Combination Blockade of TL1A and IL23 in Tissue Explants from IBD Patients

The disclosure shows that using an ex vivo culture system of tissue explants of intestinal biopsies from IBD patients, TL1A and IL23 signaling blockade results in differential modulation of molecular and cellular pathways, and that simultaneous blockade of both TL1A and IL23 signaling results in synergistic cellular and molecular readouts.

Conducted in this example are in vitro experiments to treat IBD tissue explant samples with a blocking antibody against TL1A alone, a blocking antibody against IL23, or a combination of a blocking antibody against TL1A and a blocking antibody against IL23. After biopsy collection from the inflamed intestine of a consenting patient donor, the fresh samples are cultured ex vivo with said blockers alone or in combinations in various concentrations. After various times, tissue samples as well as supernatants are collected and processed so as to be preserved for downstream generation of transcriptional or proteomic molecular data.

Proteomics data are generated by mesoscale diagnostics (MSD) using commercially available broad panels designed to detect protein analytes including cytokines and chemokines, as well as by ELISA and other immunoassay techniques. Gene expression molecular data are generated by processing the samples to into RNA and then generate total RNAseq data or gene expression by quantitative PCR.

Differentially expressed proteins, genes, gene sets are identified by various bioinformatic techniques including differential gene expression analysis, gene set enrichment analysis, gene set variation or pathway analysis, co-expression network analysis. These analyses serve as the basis to identify pathway-specific genes and proteins signatures, which define the molecular pathways modulated as a result of blocking either the TL1a/DR3 pathway alone, the IL23/IL23R pathway alone or both simultaneously. This approach generates pathway specific modulated genes and/or proteins and demonstrates incompletely redundant, complementary sets of genes and proteins modulated by blockade of each of the pathways as well as synergistic modulation of molecular pathways by combining these two treatments.

In addition, fresh samples are processed into single cell suspensions and followed by processing by gentle tissue dissociation so as to obtain viable single cell suspensions, which are analyzed by flow cytometry using panels of fluorescently labeled antibodies selected to evaluate expression of proteins representative of immune cell activation or down modulation, including markers for T cells, B cells and myeloid cells. Analyses of these data shows that DR3 and IL23 receptors expression on the surface of immune and stromal cells and shows some overlap but also considerable non-overlap, validating that certain cells may be activated via both pathways, while others may be activated via only one of these pathways. These data validate the rationale for targeting both pathways in order to increase the clinical efficacy observed by blockade of either pathway alone.

### Example 30: In Vivo Study Supporting Rationale for Combination Treatment of IBD with Combination Blockade of TL1a and IL23 Using the IL10−/− Model of Murine Colitis

The disclosure provides that TL1a and IL23 blockade has a synergistic effect when used in combination in the IL10−/− model of murine colitis.

IL10−/− and WT mice on the C57BL/6 background are monitored for body weight every other day. IL10−/− mice begin to manifest symptoms of colitis by 4 weeks of age compared with WT mice, including diarrhea and weight loss greater than 5%. Treatment protocols are initiated when the mice are between 14-18 weeks of age. Mice that lose more than 20% of their initial body weight or demonstrate significant morbidity as defined by established protocols are euthanized and subjected to the defined necropsy procedure.

By 14-18 weeks of age, IL10−/− mice develop sufficiently severe symptoms of disease to being testing therapeutic treatments. For four weeks, mice are treated weekly with intraperitoneal anti-TL1a and anti-IL23 injection according to Table 61. Group 1 serves as the naïve control and Group 2 is treated with isotype and vehicle controls. Groups 3 and 4 test the high dose of either TL1a or IL23 blockade, respectively. Groups 5 and 6 test the low dose of either TL1a or IL23 blockade. Groups 7 and 8 test the high and low dose combination therapies, respectively.

TABLE 61

| | | | Anti-TL1a treatment: | Anti-IL23 treatment: |
|---|---|---|---|---|
| Group | # mice | Genotype | D 0, 7, 14, 21 | D 0, 7, 14, 21 |
| 1 | 4 | WT | N/A | N/A |
| 2 | 8 | IL10−/− | 10 mpk isotype control | 10 mpk isotype control |
| 3 | 8 | IL10−/− | 10 mpk anti-TL1a | 10 mpk isotype control |
| 4 | 8 | IL10−/− | 10 mpk isotype control | 10 mpk anti-IL23 |
| 5 | 8 | IL10−/− | 2 mpk anti-TL1a | 10 mpk isotype control |
| 6 | 8 | IL10−/− | 2 mpk isotype control | 2 mpk anti-IL23 |
| 7 | 8 | IL10−/− | 10 mpk anti-TL1a | 10 mpk anti-IL23 |
| 8 | 8 | IL10−/− | 2 mpk anti-TL1a | 2 mpk anti-IL23 |

At day 28 after initiation of treatment, mice are anaesthetized and bled by cardiac puncture. Blood is kept on ice for 30 minutes and then spun down to separate the serum, which is stored at −80 C for cytokine measurement by MSD. After the terminal bleed, mice are euthanized by CO2 overdose per IACUC guidelines. Colons are harvested for each mouse and measured for length and weight. 1 cm of the middle colon is collected into RNAlater, and kept at room temperature for 2 hours prior to freezing at −80 C. The colonic tissue preserved in RNAlater is processed for mRNA and subjected to bulk RNA sequencing and standard differential gene expression analysis. The remainder of the proximal and distal colon is preserved in 10% NBF at room temperature for 48 hours prior to moving to 70% ethanol. These preserved colonic tissues are sectioned and stained by H&E prior to analysis by a veterinary pathologist for clinical signs of colitis.

Body weight loss is observed in all groups starting at ˜4 weeks of age, and leading up to treatment initiation. Continued body weight loss is observed in untreated IL10−/− mice compared to WT controls (Group 2 vs Group 1). Consistent with provided roles for TL1a/DR3 and IL23 in the IL10−/− model of colitis, treatment with anti-TL1a or anti-IL23 alleviates body weight loss in a dose-dependent manner (Groups 3-6). The disclosure provides that the non-overlapping roles of DR3 and IL23 signaling in intestinal biology result in a synergistic relationship between the two, and thus a synergistic effect of combining anti-TL1a and anti-IL23 therapies. Indeed, mice treated with the low dose of anti-TL1a and anti-IL23 together (Group 8), have reduced weight loss comparable to the high dose treatments with either anti-TL1a or anti-IL23 (Groups 3 and 4). Treating with a high dose of anti-TL1a and anti-IL23 together (Group 7) result in a complete abrogation of weight loss. The reduction of weight loss seen in mice treated with both anti-TL1a and anti-IL23 is greater than the sum of the weight loss seen in mice treated with anti-TL1A alone and that with anti-IL23 at the comparable dose, thereby confirming the unexpected synergistic effect of the combo therapy.

The intestinal pathology observed in IL10−/− mice is consistent with historical data and with the observed loss of body weight. Colonic tissue from IL10−/− mice has substantially higher histopathology scores than their WT counterparts, comprised of scoring for immune cell infiltration, gland loss, erosion, and hyperplasia. In agreement with the abrogation of body weight loss, single treatments with anti-TL1a or anti-IL23 reduces disease per histopathology in a dose dependent manner, while combination therapy with anti-TL1a and anti-IL23 gives significant protection even at low doses.

Serum cytokines are measured by mesoscale diagnostic (MSD) panel, and pro-inflammatory cytokines are substantially increased in IL10−/− mice over WT controls. Reduced levels of inflammatory cytokines are seen in the serum of mice treated with anti-TL1a or anti-IL23, and the combination therapy provides significant protection at the low dose, with even lower levels of pro-inflammatory cytokines seen with the high dose combination therapy. Similarly, the reduction of inflammatory cytokines seen in mice treated with both anti-TL1a and anti-IL23 is greater than the sum of the cytokine reduction seen in mice treated with anti-TL 1A alone and that with anti-IL23 at the comparable dose, thereby confirming the unexpected synergistic effect of the combo therapy.

To characterize the synergistic benefit of anti-TL1a and anti-IL23 combination therapy, IL10−/− mice are treated with a low dose of anti-TL1a (0.5 mpk) and an ascending titration of anti-IL23 (0.5 mpk-10 mpk), as shown in Table 62. Body weight loss is monitored and is used in conjunction with intestinal pathology to determine the lowest combination dose that significantly alleviates colitis. A lower dose of anti-IL23 that gives protection in comparison with isotype treated animals is provided by this dosing study (Group 4).

TABLE 62

| | | | Anti-TL1a treatment: | Anti-IL23 treatment: |
|---|---|---|---|---|
| Group | # mice | Genotype | D 0, 7, 14, 21, 28, 35, 42 | D 0, 7, 14, 21, 28, 35, 42 |
| 1 | 4 | WT | N/A | N/A |
| 2 | 8 | IL10−/− | 0.5 mpk isotype control | 10 mpk isotype control |
| 3 | 8 | IL10−/− | 0.5 mpk anti-TL1a | 0.5 mpk anti-IL23 |
| 4 | 8 | IL10−/− | 0.5 mpk isotype control | 1 mpk anti-IL23 |
| 5 | 8 | IL10−/− | 0.5 mpk anti-TL1a | 2.5 mpk anti-IL23 |
| 6 | 8 | IL10−/− | 0.5 mpk isotype control | 5 mpk anti-IL23 |
| 7 | 8 | IL10−/− | 0.5 mpk anti-TL1a | 10 mpk anti-IL23 |

The reciprocal experiment is also performed by treating IL10−/− mice with a low dose of anti-IL23 (0.5 mpk) and an ascending titration of anti-TL1a (0.5 mpk-10 mpk), as shown in Table 63. Again, body weight loss and intestinal pathology are used to determine the lowest combination dose that significantly alleviates induction of colitis. Compared to the isotype and vehicle control treated animals (Group 2), lower dose of anti-TL1a is required in combination with 0.5 mpk of anti-IL23 to significantly reduced T cell transfer-induced colitis (Group 5). Together, these results provide substantial increase in the potential therapeutic window by decreasing the dose of each individual drug that must be used to achieve a clinical effect.

TABLE 63

| | | | Anti-TL1a treatment: | Anti-IL23 treatment: |
|---|---|---|---|---|
| Group | # mice | Genotype | D 0, 7, 14, 21, 28, 35, 42 | D 0, 7, 14, 21, 28, 35, 42 |
| 1 | 4 | WT | N/A | N/A |
| 2 | 8 | IL10−/− | 10 mpk isotype control | 0.5 mpk isotype control |
| 3 | 8 | IL10−/− | 0.5 mpk anti-TL1a | 0.5 mpk anti-IL23 |
| 4 | 8 | IL10−/− | 1 mpk isotype control | 0.5 mpk anti-IL23 |
| 5 | 8 | IL10−/− | 2.5 mpk anti-TL1a | 0.5 mpk anti-IL23 |
| 6 | 8 | IL10−/− | 5 mpk isotype control | 0.5 mpk anti-IL23 |
| 7 | 8 | IL10−/− | 10 mpk anti-TL1a | 0.5 mpk anti-IL23 |

Additionally, RNA from the colon tissue of treated mice is sequenced and gene expression between each sample is compared by DESeq analysis. Consistent with the synergy displayed in the combination treatment with anti-TL1a and anti-IL23, and with the non-overlapping activity seen for DR3 and IL23 signaling in single cell RNAseq data, a significant non-overlap between the gene expression signature of anti-TL1a blockade and anti-IL23 blockade is observed. Gene expression analysis of gut tissue from mice treated with combinations of anti-TL1a and anti-IL23 again validates the unexpected synergy provided herein by showing that blocking the two mechanisms also results in the blockade of some genes not modulated significantly independently by blockade of either pathway alone.

In this Example, the anti-IL23 tested includes ustekinumab, guselkumab, risankizumab, brazikumab, mirikizumab, tildrakizumab, and briakinumab.

### Example 31: Combination Blockade of TL1a and IL23 in the T Cell Transfer Model of Colitis

The disclosure provides that TL1a and IL23 blockade has a synergistic effect when used in combination in the T cell transfer model of murine colitis.

To initiate colitis, CD4+CD45RB+ T cells from the spleens of C57BL/6 mice are sorted to >95% purity and transferred by I.V. injection to Rag2−/− mice lacking mature B and T cells (0.5e6 cells/mouse), which are then randomized by weight on day 0. Mice are weighed and monitored for signs of disease every other day for 7 weeks.

One day prior to the initiation of disease (day −1) and weekly throughout the course of the experiment, mice are treated with a single I.P. injection of vehicle control, anti-TL1a and/or anti-IL23 in 0.2 ml total volume according to Table 64. Group 1 serves as the naïve control and Group 2 is treated with isotype control antibodies. Groups 3 and 4 test the high dose of either anti-TL1a or anti-IL23, respectively. Groups 5 and 6 test the low dose of either anti-TL1a or anti-IL23. Groups 7 and 8 test the high and low dose combination therapies, respectively.

TABLE 64

| | | | Anti-TL1a treatment: | Anti-IL23 treatment: |
|---|---|---|---|---|
| Group | # mice | D 0 treatment | D 0, 7, 14, 21, 28, 35, 42 | D 0, 7, 14, 21, 28, 35, 42 |
| 1 | 4 | PBS control | N/A | N/A |
| 2 | 8 | 0.5e6 CD4 + CD45RB + cells | 10 mpk isotype control | 10 mpk isotype control |
| 3 | 8 | 0.5e6 CD4 + CD45RB + cells | 10 mpk anti-TL1a | 10 mpk isotype control |
| 4 | 8 | 0.5e6 CD4 + CD45RB + cells | 10 mpk isotype control | 10 mpk anti-IL23 |
| 5 | 8 | 0.5e6 CD4 + CD45RB + cells | 2 mpk anti-TL1a | 10 mpk isotype control |
| 6 | 8 | 0.5e6 CD4 + CD45RB + cells | 2 mpk isotype control | 2 mpk anti-IL23 |
| 7 | 8 | 0.5e6 CD4 + CD45RB + cells | 10 mpk anti-TL1a | 10 mpk anti-IL23 |
| 8 | 8 | 0.5e6 CD4 + CD45RB + cells | 2 mpk anti-TL1a | 2 mpk anti-IL23 |

At day 49 after disease induction with CD4+CD45RB+ T cell transfer, mice are anaesthetized and bled by cardiac puncture. Blood is kept on ice for 30 minutes and then spun down to separate the serum, which is stored at −80 C for cytokine measurement by MSD. After the terminal bleed, mice are euthanized by CO2 overdose per IACUC guidelines. Colons are harvested for each mouse and measured for length and weight. 1 cm of the middle colon is collected into RNAlater, and kept at room temperature for 2 hours prior to freezing at −80 C. The colonic tissue preserved in RNAlater is processed for mRNA and subjected to bulk RNA sequencing and differential gene expression analysis. The remainder of the proximal and distal colon is preserved in 10% NBF at room temperature for 48 hours prior to moving to 70% ethanol. These preserved colonic tissues are sectioned and stained by H&E prior to analysis by a veterinary pathologist for clinical signs of colitis.

Body weight loss is observed in T cell recipient mice compared to control Rag2−/− mice between 21 and 35 days post-transfer (Group 2 vs Group 1). Treatment with anti-TL1A or anti-IL23 alleviates body weight loss in a dose-dependent manner (Groups 3-6). The non-overlapping roles of DR3 and IL23R signaling in intestinal biology result in a synergistic relationship between the two pathways, and thus a synergistic effect of combining anti-TL1A and anti-IL23 therapies. Mice treated with the low dose of anti-TL1A and anti-IL23 together (Group 8), have reduced weight loss comparable to the high dose treatments with either anti-TL1A or anti-IL23 (Groups 3 and 4) alone. Treating with a high dose of anti-TL1A and anti-IL23 together (Group 7) results in a complete abrogation of weight loss. The reduction of weight loss seen in mice treated with both anti-TL1a and anti-IL23 is greater than the sum of the weight loss seen in mice treated with anti-TL1A alone and that with anti-IL23 at the comparable dose, thereby confirming the unexpected synergistic effect of the combo therapy.

The intestinal pathology induced by CD4+CD45RB+ cells in Rag2−/− mice is consistent with historical data and with the observed loss of body weight. CD4+CD45RB+ cells induce significant histopathology scores, which are comprised of scoring for immune cell infiltration, gland loss, erosion, and hyperplasia, indicating that the CD4+CD45RB+ cells have induced substantial colitis. In agreement with the abrogation of body weight loss, single treatment with anti-TL1A or anti-IL23 reduces disease per histopathology in a dose dependent manner, while combination therapy with anti-TL1A and anti-IL23 gives significant protection even at low doses.

Serum cytokines are measured by MSD panel, and pro-inflammatory cytokines are substantially increased in mice that receive CD4+CD45RB+ cells. Reduced levels of inflammatory cytokines are seen in the serum of mice treated with anti-TL1A or anti-IL23, and the combination therapy provides significant protection at the low dose, with even lower levels of pro-inflammatory cytokines seen with the high dose combination therapy. Similarly, the reduction of inflammatory cytokines seen in mice treated with both anti-TL1a and anti-IL23 is greater than the sum of the cytokine reduction seen in mice treated with anti-TL1A alone and that with anti-IL23 at the comparable dose, thereby confirming the unexpected synergistic effect of the combo therapy.

To characterize the synergistic benefit of anti-TL1A and anti-IL23 combination therapy, mice with T cell transfer-induced colitis are treated with a low dose of anti-TL1A (0.5 mpk) and an ascending titration of anti-IL23 (0.5 mpk-10 mpk) (Table 65). Body weight loss is monitored and is used in conjunction with intestinal pathology to determine the lowest combination dose that significantly alleviated induction of colitis. A lower dose of anti-IL23 that gives protection in comparison with isotype treated animals is provided by this dosing study (Group 4).

TABLE 65

| | | | Anti-TL1a treatment: | Anti-IL23 treatment: |
|---|---|---|---|---|
| Group | # mice | D 0 treatment | D 0, 7, 14, 21, 28, 35, 42 | D 0, 7, 14, 21, 28, 35, 42 |
| 1 | 4 | PBS control | N/A | N/A |
| 2 | 8 | 0.5e6 CD4 + CD45RB + cells | 0.5 mpk isotype control | 10 mpk isotype control |
| 3 | 8 | 0.5e6 CD4 + CD45RB + cells | 0.5 mpk anti-TL1a | 0.5 mpk anti-IL23 |
| 4 | 8 | 0.5e6 CD4 + CD45RB + cells | 0.5 mpk isotype control | 1 mpk anti-IL23 |
| 5 | 8 | 0.5e6 CD4 + CD45RB + cells | 0.5 mpk anti-TL1a | 2.5 mpk anti-IL23 |
| 6 | 8 | 0.5e6 CD4 + CD45RB + cells | 0.5 mpk isotype control | 5 mpk anti-IL23 |
| 7 | 8 | 0.5e6 CD4 + CD45RB + cells | 0.5 mpk anti-TL1a | 10 mpk anti-IL23 |

The reciprocal experiment is also performed by treating mice with T cell transfer-induced colitis with a low dose of anti-IL23 (0.5 mpk) and an ascending titration of anti-TL1A (0.5 mpk-10 mpk) (Table 66). Again, body weight loss and intestinal pathology are used to determine the lowest combination dose that significantly alleviated induction of colitis. Compared to the isotype and vehicle control treated animals (Group 2), 2.5 mpk of anti-TL1A is required in combination with 0.5 mpk of anti-IL23 (Group 5) to significantly reduce T cell transfer-induced colitis. Together, these results represent a substantial increase in the potential therapeutic window by decreasing the dose of each individual drug that must be used to achieve a clinical effect.

TABLE 66

| | | | Anti-TL1a treatment: | Anti-IL23 treatment: |
|---|---|---|---|---|
| Group | # mice | D 0 treatment | D 0, 7, 14, 21, 28, 35, 42 | D 0, 7, 14, 21, 28, 35, 42 |
| 1 | 4 | PBS control | N/A | N/A |
| 2 | 8 | 0.5e6 CD4 + CD45RB + cells | 10 mpk isotype control | 0.5 mpk isotype control |
| 3 | 8 | 0.5e6 CD4 + CD45RB + cells | 0.5 mpk anti-TL1a | 0.5 mpk anti-IL23 |
| 4 | 8 | 0.5e6 CD4 + CD45RB + cells | 1 mpk isotype control | 0.5 mpk anti-IL23 |
| 5 | 8 | 0.5e6 CD4 + CD45RB + cells | 2.5 mpk anti-TL1a | 0.5 mpk anti-IL23 |
| 6 | 8 | 0.5e6 CD4 + CD45RB + cells | 5 mpk isotype control | 0.5 mpk anti-IL23 |
| 7 | 8 | 0.5e6 CD4 + CD45RB + cells | 10 mpk anti-TL1a | 0.5 mpk anti-IL23 |

Finally, RNA from the colon tissue of treated mice is sequenced and gene expression between each sample is compared by DESeq analysis. Consistent with the synergy displayed in the combination treatment with anti-TL1A and anti-IL23, and with the non-overlapping activity seen for DR3 and IL23 signaling in single cell RNAseq data, a significant non-overlap between the gene expression signatures of anti-TL1A and anti-IL23 treatment is observed. Gene expression analysis of mice treated with combinations of anti TL1A and anti-IL-23 validates the unexpected synergy provided herein by showing that blocking the two mechanisms also results in the blockade of some genes not modulated significantly independently by blockade of either pathway alone.

In this Example, the anti-IL23 tested includes ustekinumab, guselkumab, risankizumab, brazikumab, mirikizumab, tildrakizumab, and briakinumab.

### Example 32: In Vivo Study Supporting Rationale for Combination Treatment of IBD with Combination Blockade of TL1a and IL23 Using the Anti-CD40 Model of Murine Colitis

The disclosure provides that TL1a and IL23 blockade has a synergistic effect when used in combination in the anti-CD40 model of murine colitis.

The murine anti-CD40 model of colitis is used to test the synergistic effect of blocking TL1a and IL23 on the development of colitis using a mouse-specific TL1a antibody (anti-TL1a) and a mouse-specific anti-IL23 antibody. To initiate colitis, Rag2−/− mice lacking mature B and T cells are randomized by weight and treated with 0.1 mg/mouse of anti-CD40 or isotype control antibody via I.P. injection in 0.2 ml total volume on day 0. Mice are weighed and monitored for signs of disease daily.

One day prior to the initiation of disease (day −1), mice are treated with a single I.P. injection in 0.2 ml total volume according to Table 67. Group 1 serves as the naïve control and Group 2 is treated with isotype control antibodies. Groups 3 and 4 test the high dose of either TL1a or IL23 blockade, respectively. Groups 5 and 6 test the low dose of either TL1a or IL23 blockade. Groups 7 and 8 test the high and low dose combination therapies, respectively.

TABLE 67

| Group | # mice | D 0 treatment | D −1 treatment |
|---|---|---|---|
| 1 | 4 | Untreated | |
| 2 | 4 | 0.1 mg isotype 1 | N/A |
| 3 | 8 | 0.1 mg anti-CD40 | 10 mpk isotype 2 + 10 mpk isotype 3 |
| 4 | 8 | 0.1 mg anti-CD40 | 10 mpk anti-TL1a + 10 mpk isotype 3 |
| 5 | 8 | 0.1 mg anti-CD40 | 10 mpk isotype 2 + 10 mpk anti-IL23 |
| 6 | 8 | 0.1 mg anti-CD40 | 2 mpk anti-TL1a + 2 mpk isotype 3 |
| 7 | 8 | 0.1 mg anti-CD40 | 2 mpk isotype 2 + 2 mpk anti-IL23 |
| 8 | 8 | 0.1 mg anti-CD40 | 10 mpk anti-TL1a + 10 mpk anti-IL23 |
| 9 | 8 | 0.1 mg anti-CD40 | 2 mpk anti-TL1a + 2 mpk anti-IL23 |

At day 7 after disease induction with anti-CD40, mice are anaesthetized and bled by cardiac puncture. Blood is kept on ice for 30 minutes and then spun down to separate the serum, which is stored at −80 C for cytokine measurement by MSD. After the terminal bleed, mice are euthanized by CO2 overdose per IACUC guidelines. Colons are harvested for each mouse and measured for length and weight. 1 cm of the middle colon is collected into RNAlater, and kept at room temperature for 2 hours prior to freezing at −80 C. The colonic tissue preserved in RNAlater is processed for mRNA and subjected to bulk RNA sequencing and standard differential gene expression analysis. The remainder of the proximal and distal colon are preserved in 10% NBF at room temperature for 48 hours prior to moving to 70% ethanol. These preserved colonic tissues are sectioned and stained by H&E prior to analysis by a veterinary pathologist for clinical signs of colitis.

Rapid body weight loss is observed in anti-CD40 treated mice over isotype treated controls, which plateaued at day 3 post-induction, and persisted until day 7 (Group 2 vs Group 1). Treatment with anti-TL1a or anti-IL23 alleviates body weight loss in a dose-dependent manner (Groups 3-6). The non-overlapping roles of TL1a and IL23-induced signaling in intestinal biology result in a synergistic relationship between the two pathways, and thus a synergistic effect of combining anti-TL1a and anti-IL23 therapies. Mice treated with the low dose of anti-TL1a and anti-IL23 together (Group 8), have reduced weight loss comparable to the high dose treatments with either anti-TL1a or anti-IL23 (Groups 3 and 4) alone. Treating with a high dose of anti-TL1a and anti-IL23 together (Group 7) results in a complete abrogation of weight loss. The reduction of weight loss seen in mice treated with both anti-TL1a and anti-IL23 is greater than the sum of the weight loss seen in mice treated with anti-TL1A alone and that with anti-IL23 at the comparable dose, thereby confirming the unexpected synergistic effect of the combo therapy.

The intestinal pathology observed in anti-CD40 treated mice is consistent with historical data and with the observed loss of body weight. Anti-CD40 treatment induces significant histopathology scores, which are comprised of scoring for immune cell infiltration, gland loss, erosion, and hyperplasia, indicating that the anti-CD40 treatment has induced substantial colitis. In agreement with the abrogation of body weight loss, single treatment with anti-TL1a or anti-IL23 reduce disease per histopathology in a dose dependent manner, while combination therapy with anti-TL1a and anti-IL23 gives significant protection even at low doses.

Serum cytokines are measured by MSD panel, and pro-inflammatory cytokines are substantially increased in anti-CD40 treated mice. Reduced levels of inflammatory cytokines are seen in the serum of mice treated with anti-TL1a or anti-IL23, and the combination therapy provides significant protection at the low dose, with even lower levels of pro-inflammatory cytokines seen with the high dose combination therapy. Similarly, the reduction of inflammatory cytokines seen in mice treated with both anti-TL1a and anti-IL23 is greater than the sum of the cytokine reduction seen in mice treated with anti-TL1A alone and that with anti-IL23 at the comparable dose, thereby confirming the unexpected synergistic effect of the combo therapy.

To fully establish the synergistic benefit of anti-TL1a and anti-IL23 combination therapy, mice with anti-CD40-induced colitis are treated with a low dose of anti-TL1a (0.5 mpk) and an ascending titration of anti-IL23 (0.5 mpk-10 mpk) (Table 68). Body weight loss is monitored daily and is used in conjunction with intestinal pathology to determine the lowest combination dose that significantly alleviates induction of colitis. A lower dose of anti-IL23 that gives protection in comparison with isotype treated animals is provided by this dosing study.

TABLE 68

| Group | # mice | D 0 treatment | D −1 treatment |
|---|---|---|---|
| 1 | 4 | Untreated | |
| 2 | 4 | 0.1 mg isotype 1 | N/A |
| 3 | 8 | 0.1 mg anti-CD40 | 0.5 mpk isotype 2 + 10 mpk isotype 3 |
| 4 | 8 | 0.1 mg anti-CD40 | 0.5 mpk anti-TL1a + 0.5 mpk anti-IL23 |
| 5 | 8 | 0.1 mg anti-CD40 | 0.5 mpk anti-TL1a + 1 mpk anti-IL23 |
| 6 | 8 | 0.1 mg anti-CD40 | 0.5 mpk anti-TL1a + 2.5 mpk anti-IL23 |
| 7 | 8 | 0.1 mg anti-CD40 | 0.5 mpk anti-TL1a + 5 mpk anti-IL23 |
| 8 | 8 | 0.1 mg anti-CD40 | 0.5 mpk anti-TL1a + 10 mpk anti-IL23 |

The reciprocal experiment is also performed by treating mice with anti-CD40-induced colitis with a low dose of anti-IL23 (0.5 mpk) and an ascending titration of anti-TL1a (0.5 mpk-10 mpk) (Table 69). Again, daily body weight loss and intestinal pathology are used to determine the lowest combination dose that significantly alleviates induction of colitis. Compared to the isotype control treated animals, 2.5 mpk of anti-TL1a is required in combination with 0.5 mpk of anti-IL23 to significantly reduce anti-CD40-induced colitis. Together, these results represent a substantial increase in the potential therapeutic window by decreasing the dose of each individual drug that must be used to achieve a clinical effect.

TABLE 69

| Group | # mice | D 0 treatment | D −1 treatment |
|---|---|---|---|
| 1 | 4 | Untreated | |
| 2 | 4 | 0.1 mg isotype 1 | N/A |
| 3 | 8 | 0.1 mg anti-CD40 | 10 mpk isotype 2 + 0.5 mpk isotype 3 |
| 4 | 8 | 0.1 mg anti-CD40 | 0.5 mpk anti-TL1a + 0.5 mpk anti-IL23 |
| 5 | 8 | 0.1 mg anti-CD40 | 1 mpk anti-TL1a + 0.5 mpk anti-IL23 |
| 6 | 8 | 0.1 mg anti-CD40 | 2.5 mpk anti-TL1a + 0.5 mpk anti-IL23 |
| 7 | 8 | 0.1 mg anti-CD40 | 5 mpk anti-TL1a + 0.5 mpk anti-IL23 |
| 8 | 8 | 0.1 mg anti-CD40 | 10 mpk anti-TL1a + 0.5 mpk anti-IL23 |

To further elucidate the molecular mechanisms behind the observed synergy between TL1a and IL23 blockade, RNA from the colon tissue of diseased mice treated with either 10 mpk anti-TL1a or 10 mpk anti-IL23 is sequenced and gene expression between each sample was compared by DESeq analysis. Consistent with the synergy displayed in the combination treatment with anti-TL1A and anti-IL23, and with the non-overlapping activity seen for DR3 and IL23 signaling in single cell RNAseq data, a significant non-overlap between the gene expression signatures of anti-TL1A and anti-IL23 treatment is observed. Gene expression analysis of mice treated with combinations of anti-TL1A and anti-IL23 validates the unexpected synergy provided herein by showing that blocking the two mechanisms also results in the blockade of some genes not modulated significantly independently by blockade of either pathway alone.

In this Example, the anti-IL23 tested includes ustekinumab, guselkumab, risankizumab, brazikumab, mirikizumab, tildrakizumab, and briakinumab.

### Example 31: Combination of Suboptimal Dose of TL1a and IL23 in the T Cell Transfer Model of Colitis

Before testing the combination therapy, suboptimal dose of anti-TL1A and the suboptimal dose of anti-IL23 in the T cell transfer mouse model of colitis was separately determined via dose titration studies by evaluating the dose against reduction in body weight loss in the mouse model. One of the suboptimal doses (the dose that suboptimally blocks colitis induced body weight loss) for anti-TL1A in the T cell transfer mouse model of colitis is 0.2 mg/kg. One of the suboptimal doses for anti-TL1A in the T cell transfer mouse model of colitis is 6 mg/kg. These two suboptimal doses were used in the study for the combination therapy of anti-TL1A and anti-IL23p19. The anti-TL1A used in this study was a surrogate anti-mouse TL1A antibody. The anti-IL23p19 used in this study was also a surrogate anti-mouse IL23p19 antibody (clone G23-8, BIOXCELL, at bioxcell.com/invivomab-anti-mouse-il-23-p19-be0313).

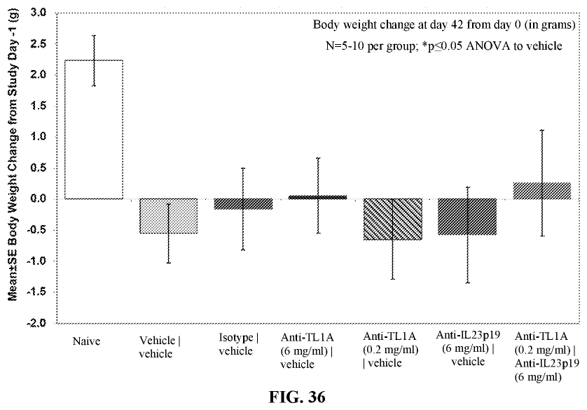

Naïve T cells from Babl/c mice were purified and transferred to Fox Chase C.B-17 SCID mice (0.4e6/mouse) by I.P. injection on study day −1 (D−1). Mice are treated with the antibody (ies) as listed in FIG. 36 by I.P. injection weekly from D0-D42. Body weight is monitored twice weekly through D49 and normalized to D−1 to determine the % change of body weight.

Some body weight loss was observed by day 42 in vehicle and isotype control treated mice compared with naïve mice. A trend toward a reduction in body weight loss was observed in mice treated with the combination of 0.2 mg/kg anti-TL1a and 6 mg/kg anti-IL-23p19 compared with vehicle, isotype control, anti-TL1A single treatment arm, or anti-IL23 single treatment arm (FIG. 36), indicating that the combination therapy of anti-TL1A and anti-IL23 trends to provide synergistic effects over each individual treatment.

TABLE 9B
Fc and Constant Regions

SEQ ID NO: 319 Light Chain Constant
RTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
SEQ ID NO: 320 IgG1 Constant
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 321 IgG1 Constant
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 322 IgG1 Constant
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 323 Fc1 (L235E)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELEGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 324 Fc2 (L235E)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELEGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 325 Fc3 (L235E)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELEGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 326 Fc4 (L234A, L235A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 327 Fc5 (L234A, L235A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 328 Fc6 (L234A, L235A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 329 Fc7 (L234A, L235A, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 330 Fc8 (L234A, L235A, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAAGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPG
SEQ ID NO: 331 Fc9 (L234A, L235A, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPG
SEQ ID NO: 332 Fc10 (L234A, L235A, P329G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALGAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 333 Fc11 (L234A, L235A, P329G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALGAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 334 Fc12 (L234A, L235A, P329G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALGAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 335 Fc13 (L234F, L235E, P331S)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEFEGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPASIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 336 Fc14 (L234F, L235E, P331S)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEFEGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPASIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 337 Fc15 (L234F, L235E, P331S)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEFEGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPASIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 338 Fc16 (L234A, L235E, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAEGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 339 Fc17 (L234A, L235E, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAEGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALP APIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPG
SEQ ID NO: 340 Fc18 (L234A, L235E, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAEGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPG
SEQ ID NO: 341 Fc19 (L234A, L235E, G237A, P331S)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAEGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPASIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPG
SEQ ID NO: 342 Fc20 (L234A, L235E, G237A, P331S)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAEGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPASIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPG
SEQ ID NO: 343 Fc21 (L234A, L235E, G237A, P331S)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAEGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPASIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPG
SEQ ID NO: 344 Fc22 (L234A, L235A, P329A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALAAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 345 Fc23 (L234A, L235A, P329A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALAAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 346 Fc24 (L234A, L235A, P329A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALAAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 347 Fc25 (D265A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVAVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 348 Fc26 (D265A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVFL
FPPKPKDTLMISRTPEVTCVVVAVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 349 Fc27 (D265A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVAVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 350 Fc28 (N297G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYGSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 351 Fc29 (N297G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPREEQYGSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 352 Fc30 (N297G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYGSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 353 Fc31 (D265A, N297A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVAVSHEDPEVKFNWYVDGVEVHNAKTKPREEQYASTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 354 Fc32 (D265A, N297A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVFL
FPPKPKDTLMISRTPEVTCVVVAVSHEDPEVKFNWYVDGVEVHNAKTKPREEQYASTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 355 Fc33 (D265A, N297A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVAVSHEDPEVKFNWYVDGVEVHNAKTKPREEQYASTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 356 Fc34 (D265A, N297G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVAVSHEDPEVKFNWYVDGVEVHNAKTKPREEQYGSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 357 Fc35 (D265A, N297G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVFL
FPPKPKDTLMISRTPEVTCVVVAVSHEDPEVKFNWYVDGVEVHNAKTKPREEQYGSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 358 Fc36 (D265A, N297G)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVAVSHEDPEVKFNWYVDGVEVHNAKTKPREEQYGSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 359 Fc37 (L235A, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELAGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 360 Fc38 (L235A, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELAGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 361 Fc39 (L235A, G237A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELAGAPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 362 Fc40 (IgG4)
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPEFLGGPSVFLFPPKP
KDTLMISRTPEVTCVVVDVSQEDPEVQFNWYVDGVEVHNAKTKPREEQFNSTYRVVSVLIV
LHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEA
LHNHYTQKSLSLSLGK
SEQ ID NO: 363 (P329A)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALAAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 364 (L234E, L235F, P331S)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEEFGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPASIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 365 (S228P)
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHE
ALHNHYTQKSLSLSLGK
SEQ ID NO: 366 (S228P, L235E)
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFEGGPSVFLFPPK
PKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHE
ALHNHYTQKSLSLSLGK
SEQ ID NO: 367 (S228P, F234A, L235A)
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEAAGGPSVFLFPPK
PKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHE
ALHNHYTQKSLSLSLGK
SEQ ID NO: 368 (L234A, L235A)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK
SEQ ID NO: 369 (L235E)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELEGGPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK
SEQ ID NO: 370 (L234A, L235A, G237A)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGAPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK
SEQ ID NO: 371 (L234A, L235E, G237A)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAEGAPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK
SEQ ID NO: 372 (L234A, L235A, P329A)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALAAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHE
ALHNHYTQKSLSLSPGK
SEQ ID NO: 373 (L234A, L235A, P329G)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALGAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHE
ALHNHYTQKSLSLSPGK
SEQ ID NO: 374 (P329A)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALAAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHE
ALHNHYTQKSLSLSPGK
SEQ ID NO: 375 (L234E, L235F, P331S)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEEFGGPSVFLFPPKPK
DTLMISRTPEVTCVVVDVSHEDPEVKFNWYVDGVEVHNAKTKPREEQYNSTYRVVSVLTV
LHQDWLNGKEYKCKVSNKALPASIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK
SEQ ID NO: 376 (D265A, N297G)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPK
DTLMISRTPEVTCVVVAVSHEDPEVKFNWYVDGVEVHNAKTKPREEQYGSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK
SEQ ID NO: 377 (N297G)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYGSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK
SEQ ID NO: 378 (S228P)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHN
HYTQKSLSLSLGK
SEQ ID NO: 379 (S228P, L235E)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHN
HYTQKSLSLSLGK
SEQ ID NO: 380 (S228P, F234A, L235A)
QVQLVQSGAEVKKPGASVKVSCKASGFDIQDTYMHWVKQRPGQGLEWMGRIDPASGHTK
YDPKFQVRVTITRDTSTSTVYLELSSLRSEDTAVYYCARSGGLPDVWGQGTTVTVSSASTKG
PSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEAAGGPSVFLFPPKPKDTL
MISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHN
HYTQKSLSLSLGK
SEQ ID NO: 381
EIVLTQSPGTLSLSPGERATLSCRASSSVSYMYWYQQKPGQAPRPLIYATSNLASGIPDRFSGS
GSGTDFTLTISRLEPEDFAVYYCQQWEGNPRTFGGGTKLEIKRTVAAPSVFIFPPSDEQLKSGT
ASVVCLLNNFYPREAKVQWKVDNALQSGNSQESVTEQDSKDSTYSLSSTLTLSKADYEKHK
VYACEVTHQGLSSPVTKSFNRGEC
SEQ ID NO: 382 (Light chain of control antibody that binds only to
TL1A trimer)
EIVLTQSPATLSLSPGERATLSCRASQSVSSYLAWYQQKPGQAPRLLIYDASNRATGI
PARFSGSGSGTDFTLTISSLEPEDFAVYYCQQRSNWPWTFGQGTKVEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
SEQ ID NO: 383 (Heavy chain of control antibody that binds only to
TL1A trimer)
QVQLVQSGAEVKKPGASVKVSCKASGYDFTYYGISWVRQAPGQGLEWMGWISTY
NGNTHYARMLQGRVTMTTDTSTRTAYMELRSLRSDDTAVYYCARENYYGSGAYR
GGMDVWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKK
VEPKSCDKTHTCPPCPAPEAAGAPSVFLFPPKPKDTLMISRTPEVTCVVVDVSHEDPE
VKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQK
SLSLSPG

The foregoing description of various embodiments known to the applicant at this time of filing the application has been presented and is intended for the purposes of illustration and description. The present description is not intended to be exhaustive nor limited to the precise form disclosed and many modifications and variations are possible in the light of the above teachings. The embodiments described serve to explain principles and practical applications, and to enable others skilled in the art to utilize the various embodiments, optionally with various modifications, as are suited to the particular use contemplated. Therefore, it is intended that the disclosure not be limited to the particular embodiments disclosed.

1. A method of treating an inflammatory disease or condition in a subject comprising administering to the subject a first composition comprising a first therapeutically effective amount of an inhibitor of tumor necrosis factor-like protein 1A (“TL1A” and such inhibitor, “TL1A inhibitor”) and administering to the subject a second composition comprising a second therapeutically effective amount of an inhibitor of interleukin 23 (“IL23” and such inhibitor, “IL23 inhibitor”).

2. A method of treating an inflammatory disease or condition in a subject comprising:
(a) administering an induction regimen to the subject comprising
(i) administering a first composition comprising a first therapeutically effective amount of a TL1A inhibitor and administering a second composition comprising a second therapeutically effective amount of an IL23 inhibitor; or
(ii) administering a composition comprising a first therapeutically effective amount of a TL1A inhibitor and a second therapeutically effective amount of an IL23 inhibitor;
and
(b) administering a maintenance regimen to the subject after the induction regimen, wherein the maintenance regimen comprises the TL1A inhibitor or the IL23 inhibitor.

3. (canceled)

4. The method of claim 2, wherein the maintenance regimen comprises a third therapeutically effective amount of the TL1A inhibitor and optionally a fourth therapeutically effective amount of the IL23 inhibitor.

5. (canceled)

6. A method of treating an inflammatory disease or condition in a subject comprising administering to the subject a composition comprising a first therapeutically effective amount of TL1A inhibitor and a second therapeutically effective amount of an IL23 inhibitor.

7. The method of claim 1, wherein the molar ratio of the first therapeutically effective amount to the second therapeutically effective amount is about 50:1, about 40:1, about 30:1, about 20:1, about 15:1, about 12:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:12, about 1:15, about 1:20, about 1:30, about 1:40, or about 1:50.

8. The method of claim 1, wherein the inflammatory disease or condition is selected from the group consisting of inflammatory bowel disease (IBD), Crohn's Disease (CD), ulcerative colitis (UC), indeterminate colitis, and moderately to severely active UC.

9-11. (canceled)

12. The method of claim 1, wherein the TL1A inhibitor is an inhibitor of TL1A expression or an inhibitor of TL1A activity,
optionally wherein the TL1A inhibitor blocks interaction of TL1A to Death Receptor 3 (“DR3”), and
optionally wherein the TL1A inhibitor is an anti-TL1A antibody or antigen binding fragment thereof.

13-15. (canceled)

16. The method of claim 12, wherein the antibody or antigen binding fragment binds to both monomeric TL1A and trimeric TL1A,
optionally wherein binding affinity of the antibody or antigen binding fragment to monomeric TL1A as measured by dissociation equilibrium constant (K_{D-monomer}) is comparable to binding affinity of the antibody or antigen binding fragment to trimeric TL1A as measured by dissociation equilibrium constant (K_{D-trimer}),
optionally wherein the KD₋ₘₒₙₒₘₑᵣ is within 1.5, 2, 3, 4, 5, 6, 7, 8, 9, or 10 fold of the KD-trimer,
optionally wherein the KD₋ₘₒₙₒₘₑᵣ is no more than 0.06 nM,
optionally wherein the KD₋ₜᵣᵢₘₑᵣ is no more than 0.06 nM, and
optionally wherein the antibody or antigen binding fragment blocks interaction of TL1A to DR3.

17-20. (canceled)

21. The method of claim 1, wherein the first therapeutically effective amount is 200 mg/dose, 250 mg/dose, 300 mg/dose, 350 mg/dose, 400 mg/dose, 450 mg/dose, 500 mg/dose, 550 mg/dose, 600 mg/dose, 650 mg/dose, 700 mg/dose, 750 mg/dose, 800 mg/dose, 850 mg/dose, 900 mg/dose, 950 mg/dose, 1000 mg/dose, 1100 mg/dose, 1200 mg/dose, 1250 mg/dose, 1300 mg/dose, 1400 mg/dose, 1500 mg/dose, 1600 mg/dose, 1700 mg/dose, 1750 mg/dose, 1800 mg/dose, 1900 mg/dose, or 2000 mg/dose,
optionally wherein the first therapeutically effective amount comprises 1, 2, 3, 4, 5, 6, 7, 8, 9, 10, 11, 12, or more doses,
optionally wherein the administering comprises administering once every 2, 4, 6, 8, 10, or 12 weeks,
optionally wherein the administering comprises administering once every 2 or 4 weeks for the first 2 administrations and then once every 2, 4, 6, or 8 weeks for the remaining administration,
optionally wherein the first therapeutically effective amount comprises 1000 mg/dose every 4 weeks, 500 mg/dose every 4 weeks, 250 mg/dose every 4 weeks, 100 mg/dose every 4 weeks, 1000 mg/dose every 2 weeks, 500 mg/dose every 2 weeks, 250 mg/dose every 2 weeks, or 100 mg/dose every 2 weeks, and
optionally wherein the first therapeutically effective amount comprises:
(i) 1000 mg/dose on week 0, 1000 mg/dose on week 2, 1000 mg/dose on week 6, and 1000 mg/dose on week 10;
(ii) 500 mg/dose on week 0, 500 mg/dose on week 2, 500 mg/dose on week 6, and 500 mg/dose on week 10;
(iii) 1000 mg/dose on week 0, 1000 mg/dose on week 2, 1000 mg/dose on week 6, and 500 mg/dose on week 10;
(iv) 1000 mg/dose on week 0, 1000 mg/dose on week 2, 500 mg/dose on week 6, and 500 mg/dose on week 10; or
(v) 1000 mg/dose on week 0, 500 mg/dose on week 2, 500 mg/dose on week 6, and 500 mg/dose on week 10.

22-27. (canceled)

28. The method of claim 16, wherein at least 91%, at least 92%, at least 93%, at least 94%, at least 95%, at least 96%, at least 97%, at least 98%, or at least 99% of the monomeric TL1A in the blood of the subject is occupied by the anti-TL1A antibody or antigen binding fragment after administering the first therapeutically effective amount, and
optionally wherein at least 91%, at least 92%, at least 93%, at least 94%, at least 95%, at least 96%, at least 97%, at least 98%, or at least 99% of the trimeric TL1A in the blood of the subject is occupied by the anti-TL1A antibody or antigen binding fragment after administering the first therapeutically effective amount.

29. (canceled)

30. A pharmaceutical composition comprising a first therapeutically effective amount of TL1A inhibitor and a second therapeutically effective amount of an IL23 inhibitor.

31. The pharmaceutical composition of claim 30, wherein the molar ratio of the first therapeutically effective amount to the second therapeutically effective amount is about 50:1, about 40:1, about 30:1, about 20:1, about 15:1, about 12:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:12, about 1:15, about 1:20, about 1:30, about 1:40, or about 1:50,
optionally wherein the TL1A inhibitor is an inhibitor of TL1A expression or an inhibitor of TL1A activity,
optionally wherein the TL1A inhibitor blocks interaction of TL1A to Death Receptor 3 (“DR3”), and
optionally wherein the TL1A inhibitor is an anti-TL1A antibody or antigen binding fragment thereof.

32-35. (canceled)

36. The pharmaceutical composition of claim 31, wherein the antibody or antigen binding fragment binds to both monomeric TL1A and trimeric TL1A,
optionally wherein binding affinity of the antibody or antigen binding fragment to monomeric TL1A as measured by dissociation equilibrium constant (K_{D-monomer}) is comparable to binding affinity of the antibody or antigen binding fragment to trimeric TL1A as measured by dissociation equilibrium constant (K_{D-trimer}),
optionally wherein the KD₋ₘₒₙₒₘₑᵣ is within 1.5, 2, 3, 4, 5, 6, 7, 8, 9, or 10 fold of the KD-ₜᵣᵢₘₑᵣ,
optionally wherein the KD₋ₘₒₙₒₘₑᵣ is no more than 0.06 nM,
optionally wherein the KD₋ₜᵣᵢₘₑᵣ is no more than 0.06 nM, and
optionally wherein the antibody or antigen binding fragment blocks interaction of TL1A to DR3.

37-41. (canceled)

42. The method of claim 16, wherein the anti-TL1A antibody or antigen binding fragment comprises a heavy chain variable region comprising: an HCDR1 comprising an amino acid sequence set forth by SEQ ID NO: 1, an HCDR2 comprising an amino acid sequence set forth by any one of SEQ ID NOS: 2-5, and an HCDR3 comprising an amino acid sequence set forth by any one of SEQ ID NOS: 6-9; and a light chain variable region comprising an LCDR1 comprising an amino acid sequence set forth by SEQ ID NO: 10, an LCDR2 comprising an amino acid sequence set forth by SEQ ID NO: 11, an LCDR3 comprising an amino acid sequence set forth by any one of SEQ ID NOS: 12-15,
optionally wherein the anti-TL1A antibody or antigen binding fragment comprises a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise no or fewer than nine amino acid modification(s) from the human IGHV1-46*02 framework and the human IGKV3-20 framework,
optionally wherein the anti-TL1A antibody or antigen binding fragment comprises a heavy chain variable domain comprising an amino acid sequence at least 96% identical to any one of SEQ ID NOS: 101-169, and a light chain variable domain comprising an amino acid sequence at least 96% identical to any one of SEQ ID NOS: 201-220, and
optionally wherein the anti-TL1A antibody or antigen binding fragment comprises a heavy chain variable region comprising SEQ ID NO: 301 XIVQLVQSGAEVKKPGASVKVSCKAS[HCDR1]WVX2QX3PGQGLEWX4G[HCDR2]RX5TX6TX7DTSTSTX8YX9ELSSLRSEDTAVYYCAR[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRESG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V, wherein HCDR1 comprises an amino acid sequence set forth by SEQ ID NO: 1, HCDR2 comprises an amino acid sequence set forth by any one of SEQ ID NOS: 2-5, HCDR3 comprises an amino acid sequence set forth by any one of SEQ ID NOS: 6-9, LCDR1 comprises an amino acid sequence set forth by SEQ ID NO: 10, LCDR2 comprises an amino acid sequence set forth by SEQ ID NO: 11, and LCDR3 comprises an amino acid sequence set forth by any one of SEQ ID NOS: 12 or 13.

43-45. (canceled)

46. The method of claim 16, wherein the IL23 inhibitor specifically inhibits IL23 and optionally does not bind to IL 12,
optionally wherein the IL23 inhibitor comprises ustekinumab and optionally wherein the second therapeutically effective amount comprises 45 mg/dose if the subject has a body weight of less than or equal to 100 kg or 90 mg/dose if the subject has a body weight of greater than 10,
optionally wherein the IL23 inhibitor comprises guselkumab and optionally wherein the second therapeutically effective amount comprises a dose of 100 mg administered in an initial dose, 4 weeks after the initial dose, and every 8 weeks after the dose at 4 weeks,
optionally wherein the IL23 inhibitor comprises risankizumab and optionally wherein the second therapeutically effective amount comprises a dose of 150 mg by subcutaneous injection at Week 0, Week 4, and every 12 weeks thereafter,
optionally wherein the IL23 inhibitor comprises brazikumab and optionally wherein the second therapeutically effective amount comprises 720-1440 mg on or about days 1, 29, and 57 delivered intravenously, followed by about 240 mg delivered subcutaneously on or about day 85 and about every 4 weeks thereafter through at least week 48,
optionally wherein the IL23 inhibitor comprises mirikizumab and optionally wherein the second therapeutically effective amount comprises at least one induction dose of about 200 mg to about 1200 mg of the mirikizumab and at least one maintenance dose of about 100 mg to about 600 mg of the mirikizumab,
optionally wherein the IL23 inhibitor comprises tildrakizumab and optionally wherein the second therapeutically effective amount comprises a dose of 100 mg of the tildrakizumab at Weeks 0, 4, and every twelve weeks thereafter up to 52 weeks, or
optionally wherein the IL23 inhibitor comprises briakinumab and optionally wherein the second therapeutically effective amount comprises (i) a first dose amount of 180 mg to 220 mg of the antibody or antigen-binding domain thereof, at week 0, and for the same first dose amount of the antibody or antigen-binding domain thereof at week 4, and (ii) a second dose amount of 80 mg to 120 mg of the antibody or antigen-binding domain thereof every 4 weeks thereafter.

47-63. (canceled)

64. The composition of claim 31, wherein the anti-TL1A antibody or antigen binding fragment comprises a heavy chain variable region comprising: an HCDR1 comprising an amino acid sequence set forth by SEQ ID NO: 1, an HCDR2 comprising an amino acid sequence set forth by any one of SEQ ID NOS: 2-5, and an HCDR3 comprising an amino acid sequence set forth by any one of SEQ ID NOS: 6-9; and a light chain variable region comprising an LCDR1 comprising an amino acid sequence set forth by SEQ ID NO: 10, an LCDR2 comprising an amino acid sequence set forth by SEQ ID NO: 11, an LCDR3 comprising an amino acid sequence set forth by any one of SEQ ID NOS: 12-15,
optionally wherein the anti-TL1A antibody or antigen binding fragment comprises a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise no or fewer than nine amino acid modification(s) from the human IGHV1-46*02 framework and the human IGKV3-20 framework,
optionally wherein the anti-TL1A antibody or antigen binding fragment comprises a heavy chain variable domain comprising an amino acid sequence at least 96% identical to any one of SEQ ID NOS: 101-169, and a light chain variable domain comprising an amino acid sequence at least 96% identical to any one of SEQ ID NOS: 201-220, and
optionally wherein the anti-TL1A antibody or antigen binding fragment comprises a heavy chain variable region comprising SEQ ID NO: 301 XIVQLVQSGAEVKKPGASVKVSCKAS[HCDR1]WVX2QX3PGQGLEWX4G[HCDR2]RX5 TX6TX7DTSTSTX8YX9ELSSLRSEDTAVYYCAR[HCDR3]WGQGTTVTVSS, and a light chain variable region comprising SEQ ID NO: 303 EIVLTQSPGTLSLSPGERATLSC[LCDR1]WYQQKPGQAPRX10X11IY[LCDR2]GIPDRFSG SGSGTDFTLTISRLEPEDFAVYYC[LCDR3]FGGGTKLEIK, wherein each of X1-X11 is independently selected from A, R, N, D, C, Q, E, G, H, I, L, K, M, F, P, S, T, W, Y, or V, wherein HCDR1 comprises an amino acid sequence set forth by SEQ ID NO: 1, HCDR2 comprises an amino acid sequence set forth by any one of SEQ ID NOS: 2-5, HCDR3 comprises an amino acid sequence set forth by any one of SEQ ID NOS: 6-9, LCDR1 comprises an amino acid sequence set forth by SEQ ID NO: 10, LCDR2 comprises an amino acid sequence set forth by SEQ ID NO: 11, and LCDR3 comprises an amino acid sequence set forth by any one of SEQ ID NOS: 12 or 13.

65. The composition of claim 31, wherein the IL23 inhibitor specifically inhibits IL23 and optionally does not bind to IL12,
optionally wherein the IL23 inhibitor comprises ustekinumab and optionally wherein the second therapeutically effective amount comprises 45 mg/dose if the subject has a body weight of less than or equal to 100 kg or 90 mg/dose if the subject has a body weight of greater than 10,
optionally wherein the IL23 inhibitor comprises guselkumab and optionally wherein the second therapeutically effective amount comprises a dose of 100 mg administered in an initial dose, 4 weeks after the initial dose, and every 8 weeks after the dose at 4 weeks,
optionally wherein the IL23 inhibitor comprises risankizumab and optionally wherein the second therapeutically effective amount comprises a dose of 150 mg by subcutaneous injection at Week 0, Week 4, and every 12 weeks thereafter,
optionally wherein the IL23 inhibitor comprises brazikumab and optionally wherein the second therapeutically effective amount comprises 720-1440 mg on or about days 1, 29, and 57 delivered intravenously, followed by about 240 mg delivered subcutaneously on or about day 85 and about every 4 weeks thereafter through at least week 48,
optionally wherein the IL23 inhibitor comprises mirikizumab and optionally wherein the second therapeutically effective amount comprises at least one induction dose of about 200 mg to about 1200 mg of the mirikizumab and at least one maintenance dose of about 100 mg to about 600 mg of the mirikizumab,
optionally wherein the IL23 inhibitor comprises tildrakizumab and optionally wherein the second therapeutically effective amount comprises a dose of 100 mg of the tildrakizumab at Weeks 0, 4, and every twelve weeks thereafter up to 52 weeks, or
optionally wherein the IL23 inhibitor comprises briakinumab and optionally wherein the second therapeutically effective amount comprises (i) a first dose amount of 180 mg to 220 mg of the antibody or antigen-binding domain thereof, at week 0, and for the same first dose amount of the antibody or antigen-binding domain thereof at week 4, and (ii) a second dose amount of 80 mg to 120 mg of the antibody or antigen-binding domain thereof every 4 weeks thereafter.
